(19) United States
(12) Patent Application Publication
(10) Pub. No.: US 2025/0154283 A1
(43) Pub. Date: May 15, 2025

(54) METHODS OF TREATING BLOOD DISORDERS WITH VWF-TARGETED MONOSPECIFIC ANTIBODIES

(71) Applicant: Hemab ApS, Copenhagen (DK)

(72) Inventors: Henrik Østergaard, Ølsted (DK); Prafull S. Gandhi, Ballerup (DK); Johan H. Faber, Vedbæk (DK); Catherine Rea, London (GB); Benny Sørensen, Walpole, MA (US)

(21) Appl. No.: 18/838,515

(22) PCT Filed: Feb. 14, 2023
(86) PCT No.: PCT/EP2023/053681

§ 371 Date: Aug. 14, 2024

Related U.S. Application Data
(60) Provisional application No. 63/269,000, filed on Mar. 8, 2022.
(60) Provisional application No. 63/268,040, filed on Feb. 15, 2022.

(51) Int. Cl. C07K 16/36; A61K 39/00; A61K 45/06

(52) U.S. Cl. CPC C07K 16/36; A61K 45/06; A61K 2039/505; C07K 2317/21; C07K 2317/32; C07K 2317/33; C07K 2317/52; C07K 2317/565; C07K 2317/92

(57) ABSTRACT

Provided herein are antibodies that bind to von Willebrand factor to an epitope positioned at a cysteine knot (CK) domain. Such antibodies are useful for methods of treatment, such methods of treating one or more blood disorders characterized by excessive bleeding.

### CROSS-REFERENCE TO RELATED APPLICATIONS

The present application claims priority to and benefit from U.S. Provisional Application Nos. 63/268,040, filed Feb. 15, 2022, and 63/269,000, filed Mar. 8, 2022, the entire contents of which are herein incorporated by reference.

### SEQUENCE LISTING

This application contains a Sequence Listing which has been submitted electronically in XML format and is hereby incorporated by reference in its entirety. Said XML copy created on Feb. 7, 2023 is named “HMT_002WO_Sequence Listing.xml” and is 457,851 bytes in size.

### SUMMARY OF THE DISCLOSURE

Disclosed herein, in certain embodiments, are compositions and methods for the treatment of blood disorders using monospecific antibodies targeted against von Willebrand factor (VWF).

Disclosed herein, in certain embodiments, are monospecific antibodies comprising an antigen-binding site that specifically binds to an epitope of a von Willebrand Factor (VWF) protein positioned at a cysteine knot (CK) domain and a fragment crystallizable (Fc) region, wherein binding of an antibody disclosed herein to the VWF protein results in accumulation of the VWF protein in blood plasma as compared to a blood plasma level of the VWF protein in the absence of the antibody. In some embodiments, an antibody disclosed herein is a monovalent antibody. In some embodiments, the Fc region is a wild-type Fc region. In some embodiments, the Fc region is a modified Fc region. In some embodiments, the modified Fc region comprises one or more mutations selected from the group consisting of a R435H mutation, N434A mutation, T252L/T253S/T254F mutation, E294delta/T307P/N434Y mutation, T256N/A378V/S383N/N434Y mutation, E294 delta mutation, M252Y/S254T/T256E mutation, M428L/N434S mutation, T307A/E380A/N434A mutation, T250Q/M428L mutation, T250Q/M428F mutation, T250E/M428F mutation, T250E/M428L mutation, T256D/Q311V/A378V mutation, T256D/H286D/T307R/Q311V/A378V mutation, H285N/T307Q/N315D mutation, T307Q/Q311V/A378V mutation, H285D/T307Q/A378V mutation, L234F/L235E/P331S mutation, L234F/L235Q/K322Q mutation, L234F/L235Q/P331G mutation, L234F/L235A/K322Q mutation, S228P/F234A/L235A/G237A/P238S mutation, L234A/L235E/G237A/A330S/P331S mutation, F243A/V264A mutation, S228P/L235E/P329G mutation, M252Y/M428L mutation, D2591/V308F mutation, T307Q/N434S mutation, M428L/V308F mutation, Q311V/N434S mutation, H433K/N434F mutation, E258F/V427T mutation, K288E/H435K mutation, F234A/L235A mutation, F234A/L235A/S228P mutation, L234A/L235A/P329G mutation, F234A/L235A/P329G mutation, P329G mutation, F234 mutation, S228P mutation, delta-K447 mutation, L235A mutation, L235E mutation, V308P mutation, V308W mutation, V308Y mutation, V308F mutation, N434S mutation, T307 mutation, Q312A mutation, K322A mutation, P329A mutation, P331A mutation, P238A mutation, D265A mutation, D269A mutation, D270A mutation, N297A mutation, A327Q mutation, P329A mutation, S239A mutation, E294A mutation, Q295A mutation, V303A mutation, A330R mutation, T299A mutation, F234A mutation, T366W mutation, T366S mutation, L368A mutation, Y407V mutation, F405L mutation, K409R mutation, F405L/R409K mutation, and any combination thereof. In some embodiments, the Fc region comprises an N-terminal truncation (e.g., a truncation by 1, 2, 3, 4, 5, 6, 7, 8, 9 10, 11, 12, 13, 14, 16, 17, 18, 19, 20, 21, 22, 23, 24, 25, 26, 27, 28, 29, 30, 31, 32, 33, 34, 35, 36, 37, 38, 39, 40, 41, 42, 43, 44, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 N-terminal residues, or more). In some embodiments, the antibody is a human IgG4 antibody comprising a first heavy chain and a second heavy chain. In some embodiments, the first heavy chain comprises a S228P/T366W/F234A/L235A/delta-K447 mutation and the second heavy chain comprises a S228P/T366S/L368A/Y407V/F234A/L235A/delta-K447 mutation. In some embodiments, the first heavy chain comprises a S228P/T366W/delta-K447 mutation and the second heavy chain comprises a S228P/T366S/L368A/Y407V/delta-K447 mutation. In some embodiments, the first heavy chain and the second heavy chain further comprise a M252Y/S254T/T256E mutation. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 12 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 16. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 12 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 16. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 12 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 16. In some embodiments, the antibody comprises a variable heavy chain (VH) region having an amino acid sequence with at least 95% (e.g., at least 95%, 96%, 97%, 98%, 99%, or more) identity to SEQ ID NO: 12 and a variable light chain (VL) region having an amino acid sequence with at least 95% (e.g., at least 95%, 96%, 97%, 98%, 99%, or more) identity to SEQ ID NO: 16. In some embodiments, the antibody comprises a variable heavy chain (VH) region having an amino acid sequence 100% identical to the amino acid sequence of SEQ ID NO: 12 and a variable light chain (VL) region having an amino acid sequence 100% identical to the amino acid sequence of SEQ ID NO: 16. In some embodiments, the antibody comprises a VH comprising therein a complementarity determining region (CDR) H1 having an amino acid sequence 100% identical to the amino acid sequence of SEQ ID NO: 13, a CDR H2 having an amino acid sequence 100% identical to the amino acid sequence of SEQ ID NO: 14, and a CDR H3 having an amino acid sequence 100% identical to the amino acid sequence of SEQ ID NO: 15; and a VL comprising therein a complementarity determining region (CDR) L1 having an amino acid sequence 100% identical to the amino acid sequence of SEQ ID NO: 17, a CDR L2 having an amino acid sequence 100% identical to the amino acid sequence of KAS, and a CDR L3 having an amino acid sequence 100% identical to the amino acid sequence of SEQ ID NO: 18. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 12 and comprising therein a complementarity determining region (CDR) H1 having an amino acid sequence 100% identical to the amino acid sequence of SEQ ID NO: 13, a CDR H2 having an amino acid sequence 100% identical to the amino acid sequence of SEQ ID NO: 14, and a CDR H3 having an amino acid sequence 100% identical to the amino acid sequence of SEQ ID NO: 15; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 16 and comprising therein a complementarity determining region (CDR) L1 having an amino acid sequence 100% identical to the amino acid sequence of SEQ ID NO: 17, a CDR L2 having an amino acid sequence 100% identical to the amino acid sequence of KAS, and a CDR L3 having an amino acid sequence 100% identical to the amino acid sequence of SEQ ID NO: 18. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 453, a second heavy chain region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 473, and a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 463. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 19 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 23. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 19 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 23. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 19 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 23. In some embodiments, the antibody comprises a variable heavy chain (VH) region having an amino acid sequence with at least 95% (e.g., at least 95%, 96%, 97%, 98%, 99%, or more) identity to SEQ ID NO: 19 and a variable light chain (VL) region having an amino acid sequence with at least 95% (e.g., at least 95%, 96%, 97%, 98%, 99%, or more) identity to SEQ ID NO: 23. In some embodiments, the antibody comprises a variable heavy chain (VH) region having an amino acid sequence 100% identical to the amino acid sequence of SEQ ID NO: 19 and a variable light chain (VL) region having an amino acid sequence 100% identical to the amino acid sequence of SEQ ID NO: 23. In some embodiments, the antibody comprises a VH comprising therein a complementarity determining region (CDR) H1 having an amino acid sequence 100% identical to the amino acid sequence of SEQ ID NO: 20, a CDR H2 having an amino acid sequence 100% identical to the amino acid sequence of SEQ ID NO: 21, and a CDR H3 having an amino acid sequence 100% identical to the amino acid sequence of SEQ ID NO: 22; and a VL comprising therein a complementarity determining region (CDR) L1 having an amino acid sequence 100% identical to the amino acid sequence of SEQ ID NO: 24, a CDR L2 having an amino acid sequence 100% identical to the amino acid sequence of AAS, and a CDR L3 having an amino acid sequence 100% identical to the amino acid sequence of SEQ ID NO: 25. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 19 and comprising therein a complementarity determining region (CDR) H1 having an amino acid sequence 100% identical to the amino acid sequence of SEQ ID NO: 20, a CDR H2 having an amino acid sequence 100% identical to the amino acid sequence of SEQ ID NO: 21, and a CDR H3 having an amino acid sequence 100% identical to the amino acid sequence of SEQ ID NO: 22; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 23 and comprising therein a complementarity determining region (CDR) L1 having an amino acid sequence 100% identical to the amino acid sequence of SEQ ID NO: 24, a CDR L2 having an amino acid sequence 100% identical to the amino acid sequence of AAS, and a CDR L3 having an amino acid sequence 100% identical to the amino acid sequence of SEQ ID NO: 25. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 454, a second heavy chain region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 474, and a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 464. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 26 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 30. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 26 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 30. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 26 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 30. In some embodiments, the antibody comprises a variable heavy chain (VH) region having an amino acid sequence with at least 95% (e.g., at least 95%, 96%, 97%, 98%, 99%, or more) identity to SEQ ID NO: 26 and a variable light chain (VL) region having an amino acid sequence with at least 95% (e.g., at least 95%, 96%, 97%, 98%, 99%, or more) identity to SEQ ID NO: 30. In some embodiments, the antibody comprises a variable heavy chain (VH) region having an amino acid sequence 100% identical to the amino acid sequence of SEQ ID NO: 26 and a variable light chain (VL) region having an amino acid sequence 100% identical to the amino acid sequence of SEQ ID NO: 30. In some embodiments, the antibody comprises a VH comprising therein a complementarity determining region (CDR) H1 having an amino acid sequence 100% identical to the amino acid sequence of SEQ ID NO: 27, a CDR H2 having an amino acid sequence 100% identical to the amino acid sequence of SEQ ID NO: 28, and a CDR H3 having an amino acid sequence 100% identical to the amino acid sequence of SEQ ID NO: 29; and a VL comprising therein a complementarity determining region (CDR) L1 having an amino acid sequence 100% identical to the amino acid sequence of SEQ ID NO: 31, a CDR L2 having an amino acid sequence 100% identical to the amino acid sequence of ESS, and a CDR L3 having an amino acid sequence 100% identical to the amino acid sequence of SEQ ID NO: 32. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 26 and comprising therein a complementarity determining region (CDR) H1 having an amino acid sequence 100% identical to the amino acid sequence of SEQ ID NO: 27, a CDR H2 having an amino acid sequence 100% identical to the amino acid sequence of SEQ ID NO: 28, and a CDR H3 having an amino acid sequence 100% identical to the amino acid sequence of SEQ ID NO: 29; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 30 and comprising therein a complementarity determining region (CDR) L1 having an amino acid sequence 100% identical to the amino acid sequence of SEQ ID NO: 31, a CDR L2 having an amino acid sequence 100% identical to the amino acid sequence of ESS, and a CDR L3 having an amino acid sequence 100% identical to the amino acid sequence of SEQ ID NO: 32. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 33 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 37. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 33 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 37. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 33 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 37. In some embodiments, the antibody comprises a variable heavy chain (VH) region having an amino acid sequence with at least 95% (e.g., at least 95%, 96%, 97%, 98%, 99%, or more) identity to SEQ ID NO: 33 and a variable light chain (VL) region having an amino acid sequence with at least 95% (e.g., at least 95%, 96%, 97%, 98%, 99%, or more) identity to SEQ ID NO: 37. In some embodiments, the antibody comprises a variable heavy chain (VH) region having an amino acid sequence 100% identical to the amino acid sequence of SEQ ID NO: 33 and a variable light chain (VL) region having an amino acid sequence 100% identical to the amino acid sequence of SEQ ID NO: 37. In some embodiments, the antibody comprises a VH comprising therein a complementarity determining region (CDR) H1 having an amino acid sequence 100% identical to the amino acid sequence of SEQ ID NO: 34, a CDR H2 having an amino acid sequence 100% identical to the amino acid sequence of SEQ ID NO: 35, and a CDR H3 having an amino acid sequence 100% identical to the amino acid sequence of SEQ ID NO: 36; and a VL comprising therein a complementarity determining region (CDR) L1 having an amino acid sequence 100% identical to the amino acid sequence of SEQ ID NO: 38, a CDR L2 having an amino acid sequence 100% identical to the amino acid sequence of DAT, and a CDR L3 having an amino acid sequence 100% identical to the amino acid sequence of SEQ ID NO: 39. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 33 and comprising therein a complementarity determining region (CDR) H1 having an amino acid sequence 100% identical to the amino acid sequence of SEQ ID NO: 34, a CDR H2 having an amino acid sequence 100% identical to the amino acid sequence of SEQ ID NO: 35, and a CDR H3 having an amino acid sequence 100% identical to the amino acid sequence of SEQ ID NO: 36; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 37 and comprising therein a complementarity determining region (CDR) L1 having an amino acid sequence 100% identical to the amino acid sequence of SEQ ID NO: 38, a CDR L2 having an amino acid sequence 100% identical to the amino acid sequence of DAT, and a CDR L3 having an amino acid sequence 100% identical to the amino acid sequence of SEQ ID NO: 39. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 455, a second heavy chain region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 475, and a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 465. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 40 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 44. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 40 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 44. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 40 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 44. In some embodiments, the antibody comprises a variable heavy chain (VH) region having an amino acid sequence with at least 95% (e.g., at least 95%, 96%, 97%, 98%, 99%, or more) identity to SEQ ID NO: 40 and a variable light chain (VL) region having an amino acid sequence with at least 95% (e.g., at least 95%, 96%, 97%, 98%, 99%, or more) identity to SEQ ID NO: 44. In some embodiments, the antibody comprises a variable heavy chain (VH) region having an amino acid sequence 100% identical to the amino acid sequence of SEQ ID NO: 40 and a variable light chain (VL) region having an amino acid sequence 100% identical to the amino acid sequence of SEQ ID NO: 44. In some embodiments, the antibody comprises a VH comprising therein a complementarity determining region (CDR) H1 having an amino acid sequence 100% identical to the amino acid sequence of SEQ ID NO: 41, a CDR H2 having an amino acid sequence 100% identical to the amino acid sequence of SEQ ID NO: 42, and a CDR H3 having an amino acid sequence 100% identical to the amino acid sequence of SEQ ID NO: 43; and a VL comprising therein a complementarity determining region (CDR) L1 having an amino acid sequence 100% identical to the amino acid sequence of SEQ ID NO: 45, a CDR L2 having an amino acid sequence 100% identical to the amino acid sequence of GSS, and a CDR L3 having an amino acid sequence 100% identical to the amino acid sequence of SEQ ID NO: 46. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 40 and comprising therein a complementarity determining region (CDR) H1 having an amino acid sequence 100% identical to the amino acid sequence of SEQ ID NO: 41, a CDR H2 having an amino acid sequence 100% identical to the amino acid sequence of SEQ ID NO: 42, and a CDR H3 having an amino acid sequence 100% identical to the amino acid sequence of SEQ ID NO: 43; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 44 and comprising therein a complementarity determining region (CDR) L1 having an amino acid sequence 100% identical to the amino acid sequence of SEQ ID NO: 45, a CDR L2 having an amino acid sequence 100% identical to the amino acid sequence of SEQ ID GSS, and a CDR L3 having an amino acid sequence 100% identical to the amino acid sequence of SEQ ID NO: 46. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 456, a second heavy chain region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 476, and a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 466. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 47 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 51. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 47 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 51. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 47 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 51. In some embodiments, the antibody comprises a variable heavy chain (VH) region having an amino acid sequence with at least 95% (e.g., at least 95%, 96%, 97%, 98%, 99%, or more) identity to SEQ ID NO: 47 and a variable light chain (VL) region having an amino acid sequence with at least 95% (e.g., at least 95%, 96%, 97%, 98%, 99%, or more) identity to SEQ ID NO: 51. In some embodiments, the antibody comprises a variable heavy chain (VH) region having an amino acid sequence 100% identical to the amino acid sequence of SEQ ID NO: 47 and a variable light chain (VL) region having an amino acid sequence 100% identical to the amino acid sequence of SEQ ID NO: 51. In some embodiments, the antibody comprises a VH comprising therein a complementarity determining region (CDR) H1 having an amino acid sequence 100% identical to the amino acid sequence of SEQ ID NO: 48, a CDR H2 having an amino acid sequence 100% identical to the amino acid sequence of SEQ ID NO: 49, and a CDR H3 having an amino acid sequence 100% identical to the amino acid sequence of SEQ ID NO: 50; and a VL comprising therein a complementarity determining region (CDR) L1 having an amino acid sequence 100% identical to the amino acid sequence of SEQ ID NO: 52, a CDR L2 having an amino acid sequence 100% identical to the amino acid sequence of DAT, and a CDR L3 having an amino acid sequence 100% identical to the amino acid sequence of SEQ ID NO: 53. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 47 and comprising therein a complementarity determining region (CDR) H1 having an amino acid sequence 100% identical to the amino acid sequence of SEQ ID NO: 48, a CDR H2 having an amino acid sequence 100% identical to the amino acid sequence of SEQ ID NO: 49, and a CDR H3 having an amino acid sequence 100% identical to the amino acid sequence of SEQ ID NO: 50; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 51 and comprising therein a complementarity determining region (CDR) L1 having an amino acid sequence 100% identical to the amino acid sequence of SEQ ID NO: 52, a CDR L2 having an amino acid sequence 100% identical to the amino acid sequence of DAT, and a CDR L3 having an amino acid sequence 100% identical to the amino acid sequence of SEQ ID NO: 53. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 54 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 58. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 54 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 58. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 54 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 58. In some embodiments, the antibody comprises a variable heavy chain (VH) region having an amino acid sequence with at least 95% (e.g., at least 95%, 96%, 97%, 98%, 99%, or more) identity to SEQ ID NO: 54 and a variable light chain (VL) region having an amino acid sequence with at least 95% (e.g., at least 95%, 96%, 97%, 98%, 99%, or more) identity to SEQ ID NO: 58. In some embodiments, the antibody comprises a variable heavy chain (VH) region having an amino acid sequence 100% identical to the amino acid sequence of SEQ ID NO: 54 and a variable light chain (VL) region having an amino acid sequence 100% identical to the amino acid sequence of SEQ ID NO: 58. In some embodiments, the antibody comprises a VH comprising therein a complementarity determining region (CDR) H1 having an amino acid sequence 100% identical to the amino acid sequence of SEQ ID NO: 55, a CDR H2 having an amino acid sequence 100% identical to the amino acid sequence of SEQ ID NO: 56, and a CDR H3 having an amino acid sequence 100% identical to the amino acid sequence of SEQ ID NO: 57; and a VL comprising therein a complementarity determining region (CDR) L1 having an amino acid sequence 100% identical to the amino acid sequence of SEQ ID NO: 59, a CDR L2 having an amino acid sequence 100% identical to the amino acid sequence of GAS, and a CDR L3 having an amino acid sequence 100% identical to the amino acid sequence of SEQ ID NO: 60. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 54 and comprising therein a complementarity determining region (CDR) H1 having an amino acid sequence 100% identical to the amino acid sequence of SEQ ID NO: 55, a CDR H2 having an amino acid sequence 100% identical to the amino acid sequence of SEQ ID NO: 56, and a CDR H3 having an amino acid sequence 100% identical to the amino acid sequence of SEQ ID NO: 57; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 58 and comprising therein a complementarity determining region (CDR) L1 having an amino acid sequence 100% identical to the amino acid sequence of SEQ ID NO: 59, a CDR L2 having an amino acid sequence 100% identical to the amino acid sequence of GAS, and a CDR L3 having an amino acid sequence 100% identical to the amino acid sequence of SEQ ID NO: 60. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 457, a second heavy chain region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 477, and a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 467. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 61 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 65. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 61 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 65. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 61 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 65. In some embodiments, the antibody comprises a variable heavy chain (VH) region having an amino acid sequence with at least 95% (e.g., at least 95%, 96%, 97%, 98%, 99%, or more) identity to SEQ ID NO: 61 and a variable light chain (VL) region having an amino acid sequence with at least 95% (e.g., at least 95%, 96%, 97%, 98%, 99%, or more) identity to SEQ ID NO: 65. In some embodiments, the antibody comprises a variable heavy chain (VH) region having an amino acid sequence 100% identical to the amino acid sequence of SEQ ID NO: 61 and a variable light chain (VL) region having an amino acid sequence 100% identical to the amino acid sequence of SEQ ID NO: 65. In some embodiments, the antibody comprises a VH comprising therein a complementarity determining region (CDR) H1 having an amino acid sequence 100% identical to the amino acid sequence of SEQ ID NO: 62, a CDR H2 having an amino acid sequence 100% identical to the amino acid sequence of SEQ ID NO: 63, and a CDR H3 having an amino acid sequence 100% identical to the amino acid sequence of SEQ ID NO: 64; and a VL comprising therein a complementarity determining region (CDR) L1 having an amino acid sequence 100% identical to the amino acid sequence of SEQ ID NO: 66, a CDR L2 having an amino acid sequence 100% identical to the amino acid sequence of DAS, and a CDR L3 having an amino acid sequence 100% identical to the amino acid sequence of SEQ ID NO: 67. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 61 and comprising therein a complementarity determining region (CDR) H1 having an amino acid sequence 100% identical to the amino acid sequence of SEQ ID NO: 62, a CDR H2 having an amino acid sequence 100% identical to the amino acid sequence of SEQ ID NO: 63, and a CDR H3 having an amino acid sequence 100% identical to the amino acid sequence of SEQ ID NO: 64; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 65 and comprising therein a complementarity determining region (CDR) L1 having an amino acid sequence 100% identical to the amino acid sequence of SEQ ID NO: 66, a CDR L2 having an amino acid sequence 100% identical to the amino acid sequence of DAS, and a CDR L3 having an amino acid sequence 100% identical to the amino acid sequence of SEQ ID NO: 67. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 68 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 72. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 68 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 72. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 68 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 72. In some embodiments, the antibody comprises a variable heavy chain (VH) region having an amino acid sequence with at least 95% (e.g., at least 95%, 96%, 97%, 98%, 99%, or more) identity to SEQ ID NO: 68 and a variable light chain (VL) region having an amino acid sequence with at least 95% (e.g., at least 95%, 96%, 97%, 98%, 99%, or more) identity to SEQ ID NO: 72. In some embodiments, the antibody comprises a variable heavy chain (VH) region having an amino acid sequence 100% identical to the amino acid sequence of SEQ ID NO: 68 and a variable light chain (VL) region having an amino acid sequence 100% identical to the amino acid sequence of SEQ ID NO: 72. In some embodiments, the antibody comprises a VH comprising therein a complementarity determining region (CDR) H1 having an amino acid sequence 100% identical to the amino acid sequence of SEQ ID NO: 69, a CDR H2 having an amino acid sequence 100% identical to the amino acid sequence of SEQ ID NO: 70, and a CDR H3 having an amino acid sequence 100% identical to the amino acid sequence of SEQ ID NO: 71; and a VL comprising therein a complementarity determining region (CDR) L1 having an amino acid sequence 100% identical to the amino acid sequence of SEQ ID NO: 73, a CDR L2 having an amino acid sequence 100% identical to the amino acid sequence of GAS, and a CDR L3 having an amino acid sequence 100% identical to the amino acid sequence of SEQ ID NO: 74. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 68 and comprising therein a complementarity determining region (CDR) H1 having an amino acid sequence 100% identical to the amino acid sequence of SEQ ID NO: 69, a CDR H2 having an amino acid sequence 100% identical to the amino acid sequence of SEQ ID NO: 70, and a CDR H3 having an amino acid sequence 100% identical to the amino acid sequence of SEQ ID NO: 71; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 72 and comprising therein a complementarity determining region (CDR) L1 having an amino acid sequence 100% identical to the amino acid sequence of SEQ ID NO: 73, a CDR L2 having an amino acid sequence 100% identical to the amino acid sequence of GAS, and a CDR L3 having an amino acid sequence 100% identical to the amino acid sequence of SEQ ID NO: 74. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 75 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 79. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 75 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 79. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 75 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 79. In some embodiments, the antibody comprises a variable heavy chain (VH) region having an amino acid sequence with at least 95% (e.g., at least 95%, 96%, 97%, 98%, 99%, or more) identity to SEQ ID NO: 75 and a variable light chain (VL) region having an amino acid sequence with at least 95% (e.g., at least 95%, 96%, 97%, 98%, 99%, or more) identity to SEQ ID NO: 79. In some embodiments, the antibody comprises a variable heavy chain (VH) region having an amino acid sequence 100% identical to the amino acid sequence of SEQ ID NO: 75 and a variable light chain (VL) region having an amino acid sequence 100% identical to the amino acid sequence of SEQ ID NO: 79. In some embodiments, the antibody comprises a VH comprising therein a complementarity determining region (CDR) H1 having an amino acid sequence 100% identical to the amino acid sequence of SEQ ID NO: 76, a CDR H2 having an amino acid sequence 100% identical to the amino acid sequence of SEQ ID NO: 77, and a CDR H3 having an amino acid sequence 100% identical to the amino acid sequence of SEQ ID NO: 78; and a VL comprising therein a complementarity determining region (CDR) L1 having an amino acid sequence 100% identical to the amino acid sequence of SEQ ID NO: 80, a CDR L2 having an amino acid sequence 100% identical to the amino acid sequence of HAS, and a CDR L3 having an amino acid sequence 100% identical to the amino acid sequence of SEQ ID NO: 81. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 75 and comprising therein a complementarity determining region (CDR) H1 having an amino acid sequence 100% identical to the amino acid sequence of SEQ ID NO: 76, a CDR H2 having an amino acid sequence 100% identical to the amino acid sequence of SEQ ID NO: 77, and a CDR H3 having an amino acid sequence 100% identical to the amino acid sequence of SEQ ID NO: 78; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 79 and comprising therein a complementarity determining region (CDR) L1 having an amino acid sequence 100% identical to the amino acid sequence of SEQ ID NO: 80, a CDR L2 having an amino acid sequence 100% identical to the amino acid sequence of HAS, and a CDR L3 having an amino acid sequence 100% identical to the amino acid sequence of SEQ ID NO: 81. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 82 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 86. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 82 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 86. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 82 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 86. In some embodiments, the antibody comprises a variable heavy chain (VH) region having an amino acid sequence with at least 95% (e.g., at least 95%, 96%, 97%, 98%, 99%, or more) identity to SEQ ID NO: 82 and a variable light chain (VL) region having an amino acid sequence with at least 95% (e.g., at least 95%, 96%, 97%, 98%, 99%, or more) identity to SEQ ID NO: 86. In some embodiments, the antibody comprises a variable heavy chain (VH) region having an amino acid sequence 100% identical to the amino acid sequence of SEQ ID NO: 82 and a variable light chain (VL) region having an amino acid sequence 100% identical to the amino acid sequence of SEQ ID NO: 86. In some embodiments, the antibody comprises a VH comprising therein a complementarity determining region (CDR) H1 having an amino acid sequence 100% identical to the amino acid sequence of SEQ ID NO: 83, a CDR H2 having an amino acid sequence 100% identical to the amino acid sequence of SEQ ID NO: 84, and a CDR H3 having an amino acid sequence 100% identical to the amino acid sequence of SEQ ID NO: 85; and a VL comprising therein a complementarity determining region (CDR) L1 having an amino acid sequence 100% identical to the amino acid sequence of SEQ ID NO: 87, a CDR L2 having an amino acid sequence 100% identical to the amino acid sequence of DAT, and a CDR L3 having an amino acid sequence 100% identical to the amino acid sequence of SEQ ID NO: 88. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 82 and comprising therein a complementarity determining region (CDR) H1 having an amino acid sequence 100% identical to the amino acid sequence of SEQ ID NO: 83, a CDR H2 having an amino acid sequence 100% identical to the amino acid sequence of SEQ ID NO: 84, and a CDR H3 having an amino acid sequence 100% identical to the amino acid sequence of SEQ ID NO: 85; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 86 and comprising therein a complementarity determining region (CDR) L1 having an amino acid sequence 100% identical to the amino acid sequence of SEQ ID NO: 87, a CDR L2 having an amino acid sequence 100% identical to the amino acid sequence of DAT, and a CDR L3 having an amino acid sequence 100% identical to the amino acid sequence of SEQ ID NO: 88. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 89 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 93. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 89 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 93. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 89 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 93. In some embodiments, the antibody comprises a variable heavy chain (VH) region having an amino acid sequence with at least 95% (e.g., at least 95%, 96%, 97%, 98%, 99%, or more) identity to SEQ ID NO: 89 and a variable light chain (VL) region having an amino acid sequence with at least 95% (e.g., at least 95%, 96%, 97%, 98%, 99%, or more) identity to SEQ ID NO: 93. In some embodiments, the antibody comprises a variable heavy chain (VH) region having an amino acid sequence 100% identical to the amino acid sequence of SEQ ID NO: 89 and a variable light chain (VL) region having an amino acid sequence 100% identical to the amino acid sequence of SEQ ID NO: 93. In some embodiments, the antibody comprises a VH comprising therein a complementarity determining region (CDR) H1 having an amino acid sequence 100% identical to the amino acid sequence of SEQ ID NO: 90, a CDR H2 having an amino acid sequence 100% identical to the amino acid sequence of SEQ ID NO: 91, and a CDR H3 having an amino acid sequence 100% identical to the amino acid sequence of SEQ ID NO: 92; and a VL comprising therein a complementarity determining region (CDR) L1 having an amino acid sequence 100% identical to the amino acid sequence of SEQ ID NO: 94, a CDR L2 having an amino acid sequence 100% identical to the amino acid sequence of GAS, and a CDR L3 having an amino acid sequence 100% identical to the amino acid sequence of SEQ ID NO: 95. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 89 and comprising therein a complementarity determining region (CDR) H1 having an amino acid sequence 100% identical to the amino acid sequence of SEQ ID NO: 90, a CDR H2 having an amino acid sequence 100% identical to the amino acid sequence of SEQ ID NO: 91, and a CDR H3 having an amino acid sequence 100% identical to the amino acid sequence of SEQ ID NO: 92; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 93 and comprising therein a complementarity determining region (CDR) L1 having an amino acid sequence 100% identical to the amino acid sequence of SEQ ID NO: 94, a CDR L2 having an amino acid sequence 100% identical to the amino acid sequence of GAS, and a CDR L3 having an amino acid sequence 100% identical to the amino acid sequence of SEQ ID NO: 95. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 458, a second heavy chain region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 478, and a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 468. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 96 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 100. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 96 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 100. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 96 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 100. In some embodiments, the antibody comprises a variable heavy chain (VH) region having an amino acid sequence with at least 95% (e.g., at least 95%, 96%, 97%, 98%, 99%, or more) identity to SEQ ID NO: 96 and a variable light chain (VL) region having an amino acid sequence with at least 95% (e.g., at least 95%, 96%, 97%, 98%, 99%, or more) identity to SEQ ID NO: 100. In some embodiments, the antibody comprises a variable heavy chain (VH) region having an amino acid sequence 100% identical to the amino acid sequence of SEQ ID NO: 96 and a variable light chain (VL) region having an amino acid sequence 100% identical to the amino acid sequence of SEQ ID NO: 100. In some embodiments, the antibody comprises a VH comprising therein a complementarity determining region (CDR) H1 having an amino acid sequence 100% identical to the amino acid sequence of SEQ ID NO: 97, a CDR H2 having an amino acid sequence 100% identical to the amino acid sequence of SEQ ID NO: 98, and a CDR H3 having an amino acid sequence 100% identical to the amino acid sequence of SEQ ID NO: 99; and a VL comprising therein a complementarity determining region (CDR) L1 having an amino acid sequence 100% identical to the amino acid sequence of SEQ ID NO: 101, a CDR L2 having an amino acid sequence 100% identical to the amino acid sequence of DAS, and a CDR L3 having an amino acid sequence 100% identical to the amino acid sequence of SEQ ID NO: 102. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 96 and comprising therein a complementarity determining region (CDR) H1 having an amino acid sequence 100% identical to the amino acid sequence of SEQ ID NO: 97, a CDR H2 having an amino acid sequence 100% identical to the amino acid sequence of SEQ ID NO: 98, and a CDR H3 having an amino acid sequence 100% identical to the amino acid sequence of SEQ ID NO: 99; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 100 and comprising therein a complementarity determining region (CDR) L1 having an amino acid sequence 100% identical to the amino acid sequence of SEQ ID NO: 101, a CDR L2 having an amino acid sequence 100% identical to the amino acid sequence of DAS, and a CDR L3 having an amino acid sequence 100% identical to the amino acid sequence of SEQ ID NO: 102. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 103 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 107. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 103 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 107. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 103 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 107. In some embodiments, the antibody comprises a variable heavy chain (VH) region having an amino acid sequence with at least 95% (e.g., at least 95%, 96%, 97%, 98%, 99%, or more) identity to SEQ ID NO: 103 and a variable light chain (VL) region having an amino acid sequence with at least 95% (e.g., at least 95%, 96%, 97%, 98%, 99%, or more) identity to SEQ ID NO: 107. In some embodiments, the antibody comprises a variable heavy chain (VH) region having an amino acid sequence 100% identical to the amino acid sequence of SEQ ID NO: 103 and a variable light chain (VL) region having an amino acid sequence 100% identical to the amino acid sequence of SEQ ID NO: 107. In some embodiments, the antibody comprises a VH comprising therein a complementarity determining region (CDR) H1 having an amino acid sequence 100% identical to the amino acid sequence of SEQ ID NO: 104, a CDR H2 having an amino acid sequence 100% identical to the amino acid sequence of SEQ ID NO: 105, and a CDR H3 having an amino acid sequence 100% identical to the amino acid sequence of SEQ ID NO: 106; and a VL comprising therein a complementarity determining region (CDR) L1 having an amino acid sequence 100% identical to the amino acid sequence of SEQ ID NO: 108, a CDR L2 having an amino acid sequence 100% identical to the amino acid sequence of DIS, and a CDR L3 having an amino acid sequence 100% identical to the amino acid sequence of SEQ ID NO: 109. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 103 and comprising therein a complementarity determining region (CDR) H1 having an amino acid sequence 100% identical to the amino acid sequence of SEQ ID NO: 104, a CDR H2 having an amino acid sequence 100% identical to the amino acid sequence of SEQ ID NO: 105, and a CDR H3 having an amino acid sequence 100% identical to the amino acid sequence of SEQ ID NO: 106; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 107 and comprising therein a complementarity determining region (CDR) L1 having an amino acid sequence 100% identical to the amino acid sequence of SEQ ID NO: 108, a CDR L2 having an amino acid sequence 100% identical to the amino acid sequence of DIS, and a CDR L3 having an amino acid sequence 100% identical to the amino acid sequence of SEQ ID NO: 109. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 110 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 114. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 110 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 114. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 110 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 114. In some embodiments, the antibody comprises a variable heavy chain (VH) region having an amino acid sequence with at least 95% (e.g., at least 95%, 96%, 97%, 98%, 99%, or more) identity to SEQ ID NO: 110 and a variable light chain (VL) region having an amino acid sequence with at least 95% (e.g., at least 95%, 96%, 97%, 98%, 99%, or more) identity to SEQ ID NO: 114. In some embodiments, the antibody comprises a variable heavy chain (VH) region having an amino acid sequence 100% identical to the amino acid sequence of SEQ ID NO: 110 and a variable light chain (VL) region having an amino acid sequence 100% identical to the amino acid sequence of SEQ ID NO: 114. In some embodiments, the antibody comprises a VH comprising therein a complementarity determining region (CDR) H1 having an amino acid sequence 100% identical to the amino acid sequence of SEQ ID NO: 111, a CDR H2 having an amino acid sequence 100% identical to the amino acid sequence of SEQ ID NO: 112, and a CDR H3 having an amino acid sequence 100% identical to the amino acid sequence of SEQ ID NO: 113; and a VL comprising therein a complementarity determining region (CDR) L1 having an amino acid sequence 100% identical to the amino acid sequence of SEQ ID NO: 115, a CDR L2 having an amino acid sequence 100% identical to the amino acid sequence of DAS, and a CDR L3 having an amino acid sequence 100% identical to the amino acid sequence of SEQ ID NO: 116. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 110 and comprising therein a complementarity determining region (CDR) H1 having an amino acid sequence 100% identical to the amino acid sequence of SEQ ID NO: 111, a CDR H2 having an amino acid sequence 100% identical to the amino acid sequence of SEQ ID NO: 112, and a CDR H3 having an amino acid sequence 100% identical to the amino acid sequence of SEQ ID NO: 113; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 114 and comprising therein a complementarity determining region (CDR) L1 having an amino acid sequence 100% identical to the amino acid sequence of SEQ ID NO: 115, a CDR L2 having an amino acid sequence 100% identical to the amino acid sequence of DAS, and a CDR L3 having an amino acid sequence 100% identical to the amino acid sequence of SEQ ID NO: 116. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 459, a second heavy chain region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 479, and a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 469. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 117 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 121. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 117 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 121. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 117 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 121. In some embodiments, the antibody comprises a variable heavy chain (VH) region having an amino acid sequence with at least 95% (e.g., at least 95%, 96%, 97%, 98%, 99%, or more) identity to SEQ ID NO: 117 and a variable light chain (VL) region having an amino acid sequence with at least 95% (e.g., at least 95%, 96%, 97%, 98%, 99%, or more) identity to SEQ ID NO: 121. In some embodiments, the antibody comprises a variable heavy chain (VH) region having an amino acid sequence 100% identical to the amino acid sequence of SEQ ID NO: 117 and a variable light chain (VL) region having an amino acid sequence 100% identical to the amino acid sequence of SEQ ID NO: 121. In some embodiments, the antibody comprises a VH comprising therein a complementarity determining region (CDR) H1 having an amino acid sequence 100% identical to the amino acid sequence of SEQ ID NO: 118, a CDR H2 having an amino acid sequence 100% identical to the amino acid sequence of SEQ ID NO: 119, and a CDR H3 having an amino acid sequence 100% identical to the amino acid sequence of SEQ ID NO: 120; and a VL comprising therein a complementarity determining region (CDR) L1 having an amino acid sequence 100% identical to the amino acid sequence of SEQ ID NO: 122, a CDR L2 having an amino acid sequence 100% identical to the amino acid sequence of DAS, and a CDR L3 having an amino acid sequence 100% identical to the amino acid sequence of SEQ ID NO: 123. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 117 and comprising therein a complementarity determining region (CDR) H1 having an amino acid sequence 100% identical to the amino acid sequence of SEQ ID NO: 118, a CDR H2 having an amino acid sequence 100% identical to the amino acid sequence of SEQ ID NO: 119, and a CDR H3 having an amino acid sequence 100% identical to the amino acid sequence of SEQ ID NO: 120; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 121 and comprising therein a complementarity determining region (CDR) L1 having an amino acid sequence 100% identical to the amino acid sequence of SEQ ID NO: 122, a CDR L2 having an amino acid sequence 100% identical to the amino acid sequence of DAS, and a CDR L3 having an amino acid sequence 100% identical to the amino acid sequence of SEQ ID NO: 123. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 124 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 128. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 124 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 128. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 124 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 128. In some embodiments, the antibody comprises a variable heavy chain (VH) region having an amino acid sequence with at least 95% (e.g., at least 95%, 96%, 97%, 98%, 99%, or more) identity to SEQ ID NO: 124 and a variable light chain (VL) region having an amino acid sequence with at least 95% (e.g., at least 95%, 96%, 97%, 98%, 99%, or more) identity to SEQ ID NO: 128. In some embodiments, the antibody comprises a variable heavy chain (VH) region having an amino acid sequence 100% identical to the amino acid sequence of SEQ ID NO: 124 and a variable light chain (VL) region having an amino acid sequence 100% identical to the amino acid sequence of SEQ ID NO: 128. In some embodiments, the antibody comprises a VH comprising therein a complementarity determining region (CDR) H1 having an amino acid sequence 100% identical to the amino acid sequence of SEQ ID NO: 125, a CDR H2 having an amino acid sequence 100% identical to the amino acid sequence of SEQ ID NO: 126, and a CDR H3 having an amino acid sequence 100% identical to the amino acid sequence of SEQ ID NO: 127; and a VL comprising therein a complementarity determining region (CDR) L1 having an amino acid sequence 100% identical to the amino acid sequence of SEQ ID NO: 129, a CDR L2 having an amino acid sequence 100% identical to the amino acid sequence of EAS, and a CDR L3 having an amino acid sequence 100% identical to the amino acid sequence of SEQ ID NO: 130. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 124 and comprising therein a complementarity determining region (CDR) H1 having an amino acid sequence 100% identical to the amino acid sequence of SEQ ID NO: 125, a CDR H2 having an amino acid sequence 100% identical to the amino acid sequence of SEQ ID NO: 126, and a CDR H3 having an amino acid sequence 100% identical to the amino acid sequence of SEQ ID NO: 127; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 128 and comprising therein a complementarity determining region (CDR) L1 having an amino acid sequence 100% identical to the amino acid sequence of SEQ ID NO: 129, a CDR L2 having an amino acid sequence 100% identical to the amino acid sequence of EAS, and a CDR L3 having an amino acid sequence 100% identical to the amino acid sequence of SEQ ID NO: 130. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 131 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 135. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 131 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 135. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 131 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 135. In some embodiments, the antibody comprises a variable heavy chain (VH) region having an amino acid sequence with at least 95% (e.g., at least 95%, 96%, 97%, 98%, 99%, or more) identity to SEQ ID NO: 131 and a variable light chain (VL) region having an amino acid sequence with at least 95% (e.g., at least 95%, 96%, 97%, 98%, 99%, or more) identity to SEQ ID NO: 135. In some embodiments, the antibody comprises a variable heavy chain (VH) region having an amino acid sequence 100% identical to the amino acid sequence of SEQ ID NO: 131 and a variable light chain (VL) region having an amino acid sequence 100% identical to the amino acid sequence of SEQ ID NO: 135. In some embodiments, the antibody comprises a VH comprising therein a complementarity determining region (CDR) H1 having an amino acid sequence 100% identical to the amino acid sequence of SEQ ID NO: 132, a CDR H2 having an amino acid sequence 100% identical to the amino acid sequence of SEQ ID NO: 133, and a CDR H3 having an amino acid sequence 100% identical to the amino acid sequence of SEQ ID NO: 134; and a VL comprising therein a complementarity determining region (CDR) L1 having an amino acid sequence 100% identical to the amino acid sequence of SEQ ID NO: 136, a CDR L2 having an amino acid sequence 100% identical to the amino acid sequence of DAS, and a CDR L3 having an amino acid sequence 100% identical to the amino acid sequence of SEQ ID NO: 137. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 131 and comprising therein a complementarity determining region (CDR) H1 having an amino acid sequence 100% identical to the amino acid sequence of SEQ ID NO: 132, a CDR H2 having an amino acid sequence 100% identical to the amino acid sequence of SEQ ID NO: 133, and a CDR H3 having an amino acid sequence 100% identical to the amino acid sequence of SEQ ID NO: 134; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 135 and comprising therein a complementarity determining region (CDR) L1 having an amino acid sequence 100% identical to the amino acid sequence of SEQ ID NO: 136, a CDR L2 having an amino acid sequence 100% identical to the amino acid sequence of DAS, and a CDR L3 having an amino acid sequence 100% identical to the amino acid sequence of SEQ ID NO: 137. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 138 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 142. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 138 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 142. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 138 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 142. In some embodiments, the antibody comprises a variable heavy chain (VH) region having an amino acid sequence with at least 95% (e.g., at least 95%, 96%, 97%, 98%, 99%, or more) identity to SEQ ID NO: 138 and a variable light chain (VL) region having an amino acid sequence with at least 95% (e.g., at least 95%, 96%, 97%, 98%, 99%, or more) identity to SEQ ID NO: 142. In some embodiments, the antibody comprises a variable heavy chain (VH) region having an amino acid sequence 100% identical to the amino acid sequence of SEQ ID NO: 138 and a variable light chain (VL) region having an amino acid sequence 100% identical to the amino acid sequence of SEQ ID NO: 142. In some embodiments, the antibody comprises a VH comprising therein a complementarity determining region (CDR) H1 having an amino acid sequence 100% identical to the amino acid sequence of SEQ ID NO: 139, a CDR H2 having an amino acid sequence 100% identical to the amino acid sequence of SEQ ID NO: 140, and a CDR H3 having an amino acid sequence 100% identical to the amino acid sequence of SEQ ID NO: 141; and a VL comprising therein a complementarity determining region (CDR) L1 having an amino acid sequence 100% identical to the amino acid sequence of SEQ ID NO: 143, a CDR L2 having an amino acid sequence 100% identical to the amino acid sequence of DAS, and a CDR L3 having an amino acid sequence 100% identical to the amino acid sequence of SEQ ID NO: 144. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 138 and comprising therein a complementarity determining region (CDR) H1 having an amino acid sequence 100% identical to the amino acid sequence of SEQ ID NO: 139, a CDR H2 having an amino acid sequence 100% identical to the amino acid sequence of SEQ ID NO: 140, and a CDR H3 having an amino acid sequence 100% identical to the amino acid sequence of SEQ ID NO: 141; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 142 and comprising therein a complementarity determining region (CDR) L1 having an amino acid sequence 100% identical to the amino acid sequence of SEQ ID NO: 143, a CDR L2 having an amino acid sequence 100% identical to the amino acid sequence of DAS, and a CDR L3 having an amino acid sequence 100% identical to the amino acid sequence of SEQ ID NO: 144. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 145 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 149. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 145 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 149. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 145 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 149. In some embodiments, the antibody comprises a variable heavy chain (VH) region having an amino acid sequence with at least 95% (e.g., at least 95%, 96%, 97%, 98%, 99%, or more) identity to SEQ ID NO: 145 and a variable light chain (VL) region having an amino acid sequence with at least 95% (e.g., at least 95%, 96%, 97%, 98%, 99%, or more) identity to SEQ ID NO: 149. In some embodiments, the antibody comprises a variable heavy chain (VH) region having an amino acid sequence 100% identical to the amino acid sequence of SEQ ID NO: 145 and a variable light chain (VL) region having an amino acid sequence 100% identical to the amino acid sequence of SEQ ID NO: 149. In some embodiments, the antibody comprises a VH comprising therein a complementarity determining region (CDR) H1 having an amino acid sequence 100% identical to the amino acid sequence of SEQ ID NO: 146, a CDR H2 having an amino acid sequence 100% identical to the amino acid sequence of SEQ ID NO: 147, and a CDR H3 having an amino acid sequence 100% identical to the amino acid sequence of SEQ ID NO: 148; and a VL comprising therein a complementarity determining region (CDR) L1 having an amino acid sequence 100% identical to the amino acid sequence of SEQ ID NO: 150, a CDR L2 having an amino acid sequence 100% identical to the amino acid sequence of DAS, and a CDR L3 having an amino acid sequence 100% identical to the amino acid sequence of SEQ ID NO: 151. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 145 and comprising therein a complementarity determining region (CDR) H1 having an amino acid sequence 100% identical to the amino acid sequence of SEQ ID NO: 146, a CDR H2 having an amino acid sequence 100% identical to the amino acid sequence of SEQ ID NO: 147, and a CDR H3 having an amino acid sequence 100% identical to the amino acid sequence of SEQ ID NO: 148; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 149 and comprising therein a complementarity determining region (CDR) L1 having an amino acid sequence 100% identical to the amino acid sequence of SEQ ID NO: 150, a CDR L2 having an amino acid sequence 100% identical to the amino acid sequence of DAS, and a CDR L3 having an amino acid sequence 100% identical to the amino acid sequence of SEQ ID NO: 151In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 152 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 156. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 152 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 156. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 152 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 156. In some embodiments, the antibody comprises a variable heavy chain (VH) region having an amino acid sequence with at least 95% (e.g., at least 95%, 96%, 97%, 98%, 99%, or more) identity to SEQ ID NO: 152 and a variable light chain (VL) region having an amino acid sequence with at least 95% (e.g., at least 95%, 96%, 97%, 98%, 99%, or more) identity to SEQ ID NO: 156. In some embodiments, the antibody comprises a variable heavy chain (VH) region having an amino acid sequence 100% identical to the amino acid sequence of SEQ ID NO: 152 and a variable light chain (VL) region having an amino acid sequence 100% identical to the amino acid sequence of SEQ ID NO: 156. In some embodiments, the antibody comprises a VH comprising therein a complementarity determining region (CDR) H1 having an amino acid sequence 100% identical to the amino acid sequence of SEQ ID NO: 153, a CDR H2 having an amino acid sequence 100% identical to the amino acid sequence of SEQ ID NO: 154, and a CDR H3 having an amino acid sequence 100% identical to the amino acid sequence of SEQ ID NO: 155; and a VL comprising therein a complementarity determining region (CDR) L1 having an amino acid sequence 100% identical to the amino acid sequence of SEQ ID NO: 157, a CDR L2 having an amino acid sequence 100% identical to the amino acid sequence of DAS, and a CDR L3 having an amino acid sequence 100% identical to the amino acid sequence of SEQ ID NO: 158. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 152 and comprising therein a complementarity determining region (CDR) H1 having an amino acid sequence 100% identical to the amino acid sequence of SEQ ID NO: 153, a CDR H2 having an amino acid sequence 100% identical to the amino acid sequence of SEQ ID NO: 154, and a CDR H3 having an amino acid sequence 100% identical to the amino acid sequence of SEQ ID NO: 155; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 156 and comprising therein a complementarity determining region (CDR) L1 having an amino acid sequence 100% identical to the amino acid sequence of SEQ ID NO: 157, a CDR L2 having an amino acid sequence 100% identical to the amino acid sequence of DAS, and a CDR L3 having an amino acid sequence 100% identical to the amino acid sequence of SEQ ID NO: 158. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 159 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 163. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 159 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 163. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 159 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 163. In some embodiments, the antibody comprises a variable heavy chain (VH) region having an amino acid sequence with at least 95% (e.g., at least 95%, 96%, 97%, 98%, 99%, or more) identity to SEQ ID NO: 159 and a variable light chain (VL) region having an amino acid sequence with at least 95% (e.g., at least 95%, 96%, 97%, 98%, 99%, or more) identity to SEQ ID NO: 163. In some embodiments, the antibody comprises a variable heavy chain (VH) region having an amino acid sequence 100% identical to the amino acid sequence of SEQ ID NO: 159 and a variable light chain (VL) region having an amino acid sequence 100% identical to the amino acid sequence of SEQ ID NO: 163. In some embodiments, the antibody comprises a VH comprising therein a complementarity determining region (CDR) H1 having an amino acid sequence 100% identical to the amino acid sequence of SEQ ID NO: 160, a CDR H2 having an amino acid sequence 100% identical to the amino acid sequence of SEQ ID NO: 161, and a CDR H3 having an amino acid sequence 100% identical to the amino acid sequence of SEQ ID NO: 162; and a VL comprising therein a complementarity determining region (CDR) L1 having an amino acid sequence 100% identical to the amino acid sequence of SEQ ID NO: 164, a CDR L2 having an amino acid sequence 100% identical to the amino acid sequence of DAS, and a CDR L3 having an amino acid sequence 100% identical to the amino acid sequence of SEQ ID NO: 165. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 159 and comprising therein a complementarity determining region (CDR) H1 having an amino acid sequence 100% identical to the amino acid sequence of SEQ ID NO: 160, a CDR H2 having an amino acid sequence 100% identical to the amino acid sequence of SEQ ID NO: 161, and a CDR H3 having an amino acid sequence 100% identical to the amino acid sequence of SEQ ID NO: 162; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 163 and comprising therein a complementarity determining region (CDR) L1 having an amino acid sequence 100% identical to the amino acid sequence of SEQ ID NO: 164, a CDR L2 having an amino acid sequence 100% identical to the amino acid sequence of DAS, and a CDR L3 having an amino acid sequence 100% identical to the amino acid sequence of SEQ ID NO: 165. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 166 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 170. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 166 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 170. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 166 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 170. In some embodiments, the antibody comprises a variable heavy chain (VH) region having an amino acid sequence with at least 95% (e.g., at least 95%, 96%, 97%, 98%, 99%, or more) identity to SEQ ID NO: 166 and a variable light chain (VL) region having an amino acid sequence with at least 95% (e.g., at least 95%, 96%, 97%, 98%, 99%, or more) identity to SEQ ID NO: 170. In some embodiments, the antibody comprises a variable heavy chain (VH) region having an amino acid sequence 100% identical to the amino acid sequence of SEQ ID NO: 166 and a variable light chain (VL) region having an amino acid sequence 100% identical to the amino acid sequence of SEQ ID NO: 170. In some embodiments, the antibody comprises a VH comprising therein a complementarity determining region (CDR) H1 having an amino acid sequence 100% identical to the amino acid sequence of SEQ ID NO: 167, a CDR H2 having an amino acid sequence 100% identical to the amino acid sequence of SEQ ID NO: 168, and a CDR H3 having an amino acid sequence 100% identical to the amino acid sequence of SEQ ID NO: 169; and a VL comprising therein a complementarity determining region (CDR) L1 having an amino acid sequence 100% identical to the amino acid sequence of SEQ ID NO: 171, a CDR L2 having an amino acid sequence 100% identical to the amino acid sequence of GAS, and a CDR L3 having an amino acid sequence 100% identical to the amino acid sequence of SEQ ID NO: 172. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 166 and comprising therein a complementarity determining region (CDR) H1 having an amino acid sequence 100% identical to the amino acid sequence of SEQ ID NO: 167, a CDR H2 having an amino acid sequence 100% identical to the amino acid sequence of SEQ ID NO: 168, and a CDR H3 having an amino acid sequence 100% identical to the amino acid sequence of SEQ ID NO: 169; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 170 and comprising therein a complementarity determining region (CDR) L1 having an amino acid sequence 100% identical to the amino acid sequence of SEQ ID NO: 171, a CDR L2 having an amino acid sequence 100% identical to the amino acid sequence of GAS, and a CDR L3 having an amino acid sequence 100% identical to the amino acid sequence of SEQ ID NO: 172. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 173 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 177. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 173 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 177. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 173 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 177. In some embodiments, the antibody comprises a variable heavy chain (VH) region having an amino acid sequence with at least 95% (e.g., at least 95%, 96%, 97%, 98%, 99%, or more) identity to SEQ ID NO: 173 and a variable light chain (VL) region having an amino acid sequence with at least 95% (e.g., at least 95%, 96%, 97%, 98%, 99%, or more) identity to SEQ ID NO: 177. In some embodiments, the antibody comprises a variable heavy chain (VH) region having an amino acid sequence 100% identical to the amino acid sequence of SEQ ID NO: 173 and a variable light chain (VL) region having an amino acid sequence 100% identical to the amino acid sequence of SEQ ID NO: 177. In some embodiments, the antibody comprises a VH comprising therein a complementarity determining region (CDR) H1 having an amino acid sequence 100% identical to the amino acid sequence of SEQ ID NO: 174, a CDR H2 having an amino acid sequence 100% identical to the amino acid sequence of SEQ ID NO: 175, and a CDR H3 having an amino acid sequence 100% identical to the amino acid sequence of SEQ ID NO: 176; and a VL comprising therein a complementarity determining region (CDR) L1 having an amino acid sequence 100% identical to the amino acid sequence of SEQ ID NO: 178, a CDR L2 having an amino acid sequence 100% identical to the amino acid sequence of GAS, and a CDR L3 having an amino acid sequence 100% identical to the amino acid sequence of SEQ ID NO: 179. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 173 and comprising therein a complementarity determining region (CDR) H1 having an amino acid sequence 100% identical to the amino acid sequence of SEQ ID NO: 174, a CDR H2 having an amino acid sequence 100% identical to the amino acid sequence of SEQ ID NO: 175, and a CDR H3 having an amino acid sequence 100% identical to the amino acid sequence of SEQ ID NO: 176; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 177 and comprising therein a complementarity determining region (CDR) L1 having an amino acid sequence 100% identical to the amino acid sequence of SEQ ID NO: 178, a CDR L2 having an amino acid sequence 100% identical to the amino acid sequence of GAS, and a CDR L3 having an amino acid sequence 100% identical to the amino acid sequence of SEQ ID NO: 179. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 180 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 184. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 180 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 184. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 180 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 184. In some embodiments, the antibody comprises a variable heavy chain (VH) region having an amino acid sequence with at least 95% (e.g., at least 95%, 96%, 97%, 98%, 99%, or more) identity to SEQ ID NO: 180 and a variable light chain (VL) region having an amino acid sequence with at least 95% (e.g., at least 95%, 96%, 97%, 98%, 99%, or more) identity to SEQ ID NO: 184. In some embodiments, the antibody comprises a variable heavy chain (VH) region having an amino acid sequence 100% identical to the amino acid sequence of SEQ ID NO: 180 and a variable light chain (VL) region having an amino acid sequence 100% identical to the amino acid sequence of SEQ ID NO: 184. In some embodiments, the antibody comprises a VH comprising therein a complementarity determining region (CDR) H1 having an amino acid sequence 100% identical to the amino acid sequence of SEQ ID NO: 181, a CDR H2 having an amino acid sequence 100% identical to the amino acid sequence of SEQ ID NO: 182, and a CDR H3 having an amino acid sequence 100% identical to the amino acid sequence of SEQ ID NO: 183; and a VL comprising therein a complementarity determining region (CDR) L1 having an amino acid sequence 100% identical to the amino acid sequence of SEQ ID NO: 185, a CDR L2 having an amino acid sequence 100% identical to the amino acid sequence of DVS, and a CDR L3 having an amino acid sequence 100% identical to the amino acid sequence of SEQ ID NO: 186. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 180 and comprising therein a complementarity determining region (CDR) H1 having an amino acid sequence 100% identical to the amino acid sequence of SEQ ID NO: 181, a CDR H2 having an amino acid sequence 100% identical to the amino acid sequence of SEQ ID NO: 182, and a CDR H3 having an amino acid sequence 100% identical to the amino acid sequence of SEQ ID NO: 183; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 184 and comprising therein a complementarity determining region (CDR) L1 having an amino acid sequence 100% identical to the amino acid sequence of SEQ ID NO: 185, a CDR L2 having an amino acid sequence 100% identical to the amino acid sequence of DVS, and a CDR L3 having an amino acid sequence 100% identical to the amino acid sequence of SEQ ID NO: 186. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 187 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 191. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 187 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 191. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 187 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 191. In some embodiments, the antibody comprises a variable heavy chain (VH) region having an amino acid sequence with at least 95% (e.g., at least 95%, 96%, 97%, 98%, 99%, or more) identity to SEQ ID NO: 187 and a variable light chain (VL) region having an amino acid sequence with at least 95% (e.g., at least 95%, 96%, 97%, 98%, 99%, or more) identity to SEQ ID NO: 191. In some embodiments, the antibody comprises a variable heavy chain (VH) region having an amino acid sequence 100% identical to the amino acid sequence of SEQ ID NO: 187 and a variable light chain (VL) region having an amino acid sequence 100% identical to the amino acid sequence of SEQ ID NO: 191. In some embodiments, the antibody comprises a VH comprising therein a complementarity determining region (CDR) H1 having an amino acid sequence 100% identical to the amino acid sequence of SEQ ID NO: 188, a CDR H2 having an amino acid sequence 100% identical to the amino acid sequence of SEQ ID NO: 189, and a CDR H3 having an amino acid sequence 100% identical to the amino acid sequence of SEQ ID NO: 190; and a VL comprising therein a complementarity determining region (CDR) L1 having an amino acid sequence 100% identical to the amino acid sequence of SEQ ID NO: 192, a CDR L2 having an amino acid sequence 100% identical to the amino acid sequence of DAT, and a CDR L3 having an amino acid sequence 100% identical to the amino acid sequence of SEQ ID NO: 193. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 187 and comprising therein a complementarity determining region (CDR) H1 having an amino acid sequence 100% identical to the amino acid sequence of SEQ ID NO: 188, a CDR H2 having an amino acid sequence 100% identical to the amino acid sequence of SEQ ID NO: 189, and a CDR H3 having an amino acid sequence 100% identical to the amino acid sequence of SEQ ID NO: 190; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 191 and comprising therein a complementarity determining region (CDR) L1 having an amino acid sequence 100% identical to the amino acid sequence of SEQ ID NO: 192, a CDR L2 having an amino acid sequence 100% identical to the amino acid sequence of DAT, and a CDR L3 having an amino acid sequence 100% identical to the amino acid sequence of SEQ ID NO: 193. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 194 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 198. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 194 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 198. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 194 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 198. In some embodiments, the antibody comprises a variable heavy chain (VH) region having an amino acid sequence with at least 95% (e.g., at least 95%, 96%, 97%, 98%, 99%, or more) identity to SEQ ID NO: 194 and a variable light chain (VL) region having an amino acid sequence with at least 95% (e.g., at least 95%, 96%, 97%, 98%, 99%, or more) identity to SEQ ID NO: 198. In some embodiments, the antibody comprises a variable heavy chain (VH) region having an amino acid sequence 100% identical to the amino acid sequence of SEQ ID NO: 194 and a variable light chain (VL) region having an amino acid sequence 100% identical to the amino acid sequence of SEQ ID NO: 198. In some embodiments, the antibody comprises a VH comprising therein a complementarity determining region (CDR) H1 having an amino acid sequence 100% identical to the amino acid sequence of SEQ ID NO: 195, a CDR H2 having an amino acid sequence 100% identical to the amino acid sequence of SEQ ID NO: 196, and a CDR H3 having an amino acid sequence 100% identical to the amino acid sequence of SEQ ID NO: 197; and a VL comprising therein a complementarity determining region (CDR) L1 having an amino acid sequence 100% identical to the amino acid sequence of SEQ ID NO: 199, a CDR L2 having an amino acid sequence 100% identical to the amino acid sequence of DAS, and a CDR L3 having an amino acid sequence 100% identical to the amino acid sequence of SEQ ID NO: 200. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 194 and comprising therein a complementarity determining region (CDR) H1 having an amino acid sequence 100% identical to the amino acid sequence of SEQ ID NO: 195, a CDR H2 having an amino acid sequence 100% identical to the amino acid sequence of SEQ ID NO: 196, and a CDR H3 having an amino acid sequence 100% identical to the amino acid sequence of SEQ ID NO: 197; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 198 and comprising therein a complementarity determining region (CDR) L1 having an amino acid sequence 100% identical to the amino acid sequence of SEQ ID NO: 199, a CDR L2 having an amino acid sequence 100% identical to the amino acid sequence of DAS, and a CDR L3 having an amino acid sequence 100% identical to the amino acid sequence of SEQ ID NO: 200. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 201 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 205. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 201 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 205. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 201 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 205. In some embodiments, the antibody comprises a variable heavy chain (VH) region having an amino acid sequence with at least 95% (e.g., at least 95%, 96%, 97%, 98%, 99%, or more) identity to SEQ ID NO: 201 and a variable light chain (VL) region having an amino acid sequence with at least 95% (e.g., at least 95%, 96%, 97%, 98%, 99%, or more) identity to SEQ ID NO: 205. In some embodiments, the antibody comprises a variable heavy chain (VH) region having an amino acid sequence 100% identical to the amino acid sequence of SEQ ID NO: 201 and a variable light chain (VL) region having an amino acid sequence 100% identical to the amino acid sequence of SEQ ID NO: 205. In some embodiments, the antibody comprises a VH comprising therein a complementarity determining region (CDR) H1 having an amino acid sequence 100% identical to the amino acid sequence of SEQ ID NO: 202, a CDR H2 having an amino acid sequence 100% identical to the amino acid sequence of SEQ ID NO: 203, and a CDR H3 having an amino acid sequence 100% identical to the amino acid sequence of SEQ ID NO: 204; and a VL comprising therein a complementarity determining region (CDR) L1 having an amino acid sequence 100% identical to the amino acid sequence of SEQ ID NO: 206, a CDR L2 having an amino acid sequence 100% identical to the amino acid sequence of GVD, and a CDR L3 having an amino acid sequence 100% identical to the amino acid sequence of SEQ ID NO: 207. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 201 and comprising therein a complementarity determining region (CDR) H1 having an amino acid sequence 100% identical to the amino acid sequence of SEQ ID NO: 202, a CDR H2 having an amino acid sequence 100% identical to the amino acid sequence of SEQ ID NO: 203, and a CDR H3 having an amino acid sequence 100% identical to the amino acid sequence of SEQ ID NO: 204; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 205 and comprising therein a complementarity determining region (CDR) L1 having an amino acid sequence 100% identical to the amino acid sequence of SEQ ID NO: 206, a CDR L2 having an amino acid sequence 100% identical to the amino acid sequence of GVD, and a CDR L3 having an amino acid sequence 100% identical to the amino acid sequence of SEQ ID NO: 207. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 208 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 212. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 208 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 212. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 208 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 212. In some embodiments, the antibody comprises a variable heavy chain (VH) region having an amino acid sequence with at least 95% (e.g., at least 95%, 96%, 97%, 98%, 99%, or more) identity to SEQ ID NO: 208 and a variable light chain (VL) region having an amino acid sequence with at least 95% (e.g., at least 95%, 96%, 97%, 98%, 99%, or more) identity to SEQ ID NO: 212. In some embodiments, the antibody comprises a variable heavy chain (VH) region having an amino acid sequence 100% identical to the amino acid sequence of SEQ ID NO: 208 and a variable light chain (VL) region having an amino acid sequence 100% identical to the amino acid sequence of SEQ ID NO: 212. In some embodiments, the antibody comprises a VH comprising therein a complementarity determining region (CDR) H1 having an amino acid sequence 100% identical to the amino acid sequence of SEQ ID NO: 209, a CDR H2 having an amino acid sequence 100% identical to the amino acid sequence of SEQ ID NO: 210, and a CDR H3 having an amino acid sequence 100% identical to the amino acid sequence of SEQ ID NO: 211; and a VL comprising therein a complementarity determining region (CDR) L1 having an amino acid sequence 100% identical to the amino acid sequence of SEQ ID NO: 213, a CDR L2 having an amino acid sequence 100% identical to the amino acid sequence of DAS, and a CDR L3 having an amino acid sequence 100% identical to the amino acid sequence of SEQ ID NO: 214. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 208 and comprising therein a complementarity determining region (CDR) H1 having an amino acid sequence 100% identical to the amino acid sequence of SEQ ID NO: 209, a CDR H2 having an amino acid sequence 100% identical to the amino acid sequence of SEQ ID NO: 210, and a CDR H3 having an amino acid sequence 100% identical to the amino acid sequence of SEQ ID NO: 211; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 212 and comprising therein a complementarity determining region (CDR) L1 having an amino acid sequence 100% identical to the amino acid sequence of SEQ ID NO: 213, a CDR L2 having an amino acid sequence 100% identical to the amino acid sequence of DAS, and a CDR L3 having an amino acid sequence 100% identical to the amino acid sequence of SEQ ID NO: 214. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 215 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 219. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 215 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 219. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 215 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 219. In some embodiments, the antibody comprises a variable heavy chain (VH) region having an amino acid sequence with at least 95% (e.g., at least 95%, 96%, 97%, 98%, 99%, or more) identity to SEQ ID NO: 215 and a variable light chain (VL) region having an amino acid sequence with at least 95% (e.g., at least 95%, 96%, 97%, 98%, 99%, or more) identity to SEQ ID NO: 219. In some embodiments, the antibody comprises a variable heavy chain (VH) region having an amino acid sequence 100% identical to the amino acid sequence of SEQ ID NO: 215 and a variable light chain (VL) region having an amino acid sequence 100% identical to the amino acid sequence of SEQ ID NO: 219. In some embodiments, the antibody comprises a VH comprising therein a complementarity determining region (CDR) H1 having an amino acid sequence 100% identical to the amino acid sequence of SEQ ID NO: 216, a CDR H2 having an amino acid sequence 100% identical to the amino acid sequence of SEQ ID NO: 217, and a CDR H3 having an amino acid sequence 100% identical to the amino acid sequence of SEQ ID NO: 218; and a VL comprising therein a complementarity determining region (CDR) L1 having an amino acid sequence 100% identical to the amino acid sequence of SEQ ID NO: 220, a CDR L2 having an amino acid sequence 100% identical to the amino acid sequence of GAT, and a CDR L3 having an amino acid sequence 100% identical to the amino acid sequence of SEQ ID NO: 221. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 215 and comprising therein a complementarity determining region (CDR) H1 having an amino acid sequence 100% identical to the amino acid sequence of SEQ ID NO: 216, a CDR H2 having an amino acid sequence 100% identical to the amino acid sequence of SEQ ID NO: 217, and a CDR H3 having an amino acid sequence 100% identical to the amino acid sequence of SEQ ID NO: 218; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 219 and comprising therein a complementarity determining region (CDR) L1 having an amino acid sequence 100% identical to the amino acid sequence of SEQ ID NO: 220, a CDR L2 having an amino acid sequence 100% identical to the amino acid sequence of GAT, and a CDR L3 having an amino acid sequence 100% identical to the amino acid sequence of SEQ ID NO: 221. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 222 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 226. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 222 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 226. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 222 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 226. In some embodiments, the antibody comprises a variable heavy chain (VH) region having an amino acid sequence with at least 95% (e.g., at least 95%, 96%, 97%, 98%, 99%, or more) identity to SEQ ID NO: 222 and a variable light chain (VL) region having an amino acid sequence with at least 95% (e.g., at least 95%, 96%, 97%, 98%, 99%, or more) identity to SEQ ID NO: 226. In some embodiments, the antibody comprises a variable heavy chain (VH) region having an amino acid sequence 100% identical to the amino acid sequence of SEQ ID NO: 222 and a variable light chain (VL) region having an amino acid sequence 100% identical to the amino acid sequence of SEQ ID NO: 226. In some embodiments, the antibody comprises a VH comprising therein a complementarity determining region (CDR) H1 having an amino acid sequence 100% identical to the amino acid sequence of SEQ ID NO: 223, a CDR H2 having an amino acid sequence 100% identical to the amino acid sequence of SEQ ID NO: 224, and a CDR H3 having an amino acid sequence 100% identical to the amino acid sequence of SEQ ID NO: 225; and a VL comprising therein a complementarity determining region (CDR) L1 having an amino acid sequence 100% identical to the amino acid sequence of SEQ ID NO: 227, a CDR L2 having an amino acid sequence 100% identical to the amino acid sequence of DAS, and a CDR L3 having an amino acid sequence 100% identical to the amino acid sequence of SEQ ID NO: 228. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 222 and comprising therein a complementarity determining region (CDR) H1 having an amino acid sequence 100% identical to the amino acid sequence of SEQ ID NO: 223, a CDR H2 having an amino acid sequence 100% identical to the amino acid sequence of SEQ ID NO: 224, and a CDR H3 having an amino acid sequence 100% identical to the amino acid sequence of SEQ ID NO: 225; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 226 and comprising therein a complementarity determining region (CDR) L1 having an amino acid sequence 100% identical to the amino acid sequence of SEQ ID NO: 227, a CDR L2 having an amino acid sequence 100% identical to the amino acid sequence of DAS, and a CDR L3 having an amino acid sequence 100% identical to the amino acid sequence of SEQ ID NO: 228. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 229 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 233. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 229 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 233. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 229 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 233. In some embodiments, the antibody comprises a variable heavy chain (VH) region having an amino acid sequence with at least 95% (e.g., at least 95%, 96%, 97%, 98%, 99%, or more) identity to SEQ ID NO: 229 and a variable light chain (VL) region having an amino acid sequence with at least 95% (e.g., at least 95%, 96%, 97%, 98%, 99%, or more) identity to SEQ ID NO: 233. In some embodiments, the antibody comprises a variable heavy chain (VH) region having an amino acid sequence 100% identical to the amino acid sequence of SEQ ID NO: 229 and a variable light chain (VL) region having an amino acid sequence 100% identical to the amino acid sequence of SEQ ID NO: 233. In some embodiments, the antibody comprises a VH comprising therein a complementarity determining region (CDR) H1 having an amino acid sequence 100% identical to the amino acid sequence of SEQ ID NO: 230, a CDR H2 having an amino acid sequence 100% identical to the amino acid sequence of SEQ ID NO: 231, and a CDR H3 having an amino acid sequence 100% identical to the amino acid sequence of SEQ ID NO: 232; and a VL comprising therein a complementarity determining region (CDR) L1 having an amino acid sequence 100% identical to the amino acid sequence of SEQ ID NO: 234, a CDR L2 having an amino acid sequence 100% identical to the amino acid sequence of DAS, and a CDR L3 having an amino acid sequence 100% identical to the amino acid sequence of SEQ ID NO: 235. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 229 and comprising therein a complementarity determining region (CDR) H1 having an amino acid sequence 100% identical to the amino acid sequence of SEQ ID NO: 230, a CDR H2 having an amino acid sequence 100% identical to the amino acid sequence of SEQ ID NO: 231, and a CDR H3 having an amino acid sequence 100% identical to the amino acid sequence of SEQ ID NO: 232; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 233 and comprising therein a complementarity determining region (CDR) L1 having an amino acid sequence 100% identical to the amino acid sequence of SEQ ID NO: 234, a CDR L2 having an amino acid sequence 100% identical to the amino acid sequence of DAS, and a CDR L3 having an amino acid sequence 100% identical to the amino acid sequence of SEQ ID NO: 235. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 236 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 240. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 236 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 240. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 236 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 240. In some embodiments, the antibody comprises a variable heavy chain (VH) region having an amino acid sequence with at least 95% (e.g., at least 95%, 96%, 97%, 98%, 99%, or more) identity to SEQ ID NO: 236 and a variable light chain (VL) region having an amino acid sequence with at least 95% (e.g., at least 95%, 96%, 97%, 98%, 99%, or more) identity to SEQ ID NO: 240. In some embodiments, the antibody comprises a variable heavy chain (VH) region having an amino acid sequence 100% identical to the amino acid sequence of SEQ ID NO: 236 and a variable light chain (VL) region having an amino acid sequence 100% identical to the amino acid sequence of SEQ ID NO: 240. In some embodiments, the antibody comprises a VH comprising therein a complementarity determining region (CDR) H1 having an amino acid sequence 100% identical to the amino acid sequence of SEQ ID NO: 237, a CDR H2 having an amino acid sequence 100% identical to the amino acid sequence of SEQ ID NO: 238, and a CDR H3 having an amino acid sequence 100% identical to the amino acid sequence of SEQ ID NO: 239; and a VL comprising therein a complementarity determining region (CDR) L1 having an amino acid sequence 100% identical to the amino acid sequence of SEQ ID NO: 241, a CDR L2 having an amino acid sequence 100% identical to the amino acid sequence of GAS, and a CDR L3 having an amino acid sequence 100% identical to the amino acid sequence of SEQ ID NO: 242. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 236 and comprising therein a complementarity determining region (CDR) H1 having an amino acid sequence 100% identical to the amino acid sequence of SEQ ID NO: 237, a CDR H2 having an amino acid sequence 100% identical to the amino acid sequence of SEQ ID NO: 238, and a CDR H3 having an amino acid sequence 100% identical to the amino acid sequence of SEQ ID NO: 239; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 240 and comprising therein a complementarity determining region (CDR) L1 having an amino acid sequence 100% identical to the amino acid sequence of SEQ ID NO: 241, a CDR L2 having an amino acid sequence 100% identical to the amino acid sequence of GAS, and a CDR L3 having an amino acid sequence 100% identical to the amino acid sequence of SEQ ID NO: 242. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 243 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 247. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 243 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 247. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 243 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 247. In some embodiments, the antibody comprises a variable heavy chain (VH) region having an amino acid sequence with at least 95% (e.g., at least 95%, 96%, 97%, 98%, 99%, or more) identity to SEQ ID NO: 243 and a variable light chain (VL) region having an amino acid sequence with at least 95% (e.g., at least 95%, 96%, 97%, 98%, 99%, or more) identity to SEQ ID NO: 247. In some embodiments, the antibody comprises a variable heavy chain (VH) region having an amino acid sequence 100% identical to the amino acid sequence of SEQ ID NO: 243 and a variable light chain (VL) region having an amino acid sequence 100% identical to the amino acid sequence of SEQ ID NO: 247. In some embodiments, the antibody comprises a VH comprising therein a complementarity determining region (CDR) H1 having an amino acid sequence 100% identical to the amino acid sequence of SEQ ID NO: 244, a CDR H2 having an amino acid sequence 100% identical to the amino acid sequence of SEQ ID NO: 245, and a CDR H3 having an amino acid sequence 100% identical to the amino acid sequence of SEQ ID NO: 246; and a VL comprising therein a complementarity determining region (CDR) L1 having an amino acid sequence 100% identical to the amino acid sequence of SEQ ID NO: 248, a CDR L2 having an amino acid sequence 100% identical to the amino acid sequence of GAS, and a CDR L3 having an amino acid sequence 100% identical to the amino acid sequence of SEQ ID NO: 249. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 243 and comprising therein a complementarity determining region (CDR) H1 having an amino acid sequence 100% identical to the amino acid sequence of SEQ ID NO: 244, a CDR H2 having an amino acid sequence 100% identical to the amino acid sequence of SEQ ID NO: 245, and a CDR H3 having an amino acid sequence 100% identical to the amino acid sequence of SEQ ID NO: 246; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 247 and comprising therein a complementarity determining region (CDR) L1 having an amino acid sequence 100% identical to the amino acid sequence of SEQ ID NO: 248, a CDR L2 having an amino acid sequence 100% identical to the amino acid sequence of GAS, and a CDR L3 having an amino acid sequence 100% identical to the amino acid sequence of SEQ ID NO: 249. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 460, a second heavy chain region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 480, and a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 470. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 250 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 254. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 250 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 254. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 250 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 254. In some embodiments, the antibody comprises a variable heavy chain (VH) region having an amino acid sequence with at least 95% (e.g., at least 95%, 96%, 97%, 98%, 99%, or more) identity to SEQ ID NO: 250 and a variable light chain (VL) region having an amino acid sequence with at least 95% (e.g., at least 95%, 96%, 97%, 98%, 99%, or more) identity to SEQ ID NO: 254. In some embodiments, the antibody comprises a variable heavy chain (VH) region having an amino acid sequence 100% identical to the amino acid sequence of SEQ ID NO: 250 and a variable light chain (VL) region having an amino acid sequence 100% identical to the amino acid sequence of SEQ ID NO: 254. In some embodiments, the antibody comprises a VH comprising therein a complementarity determining region (CDR) H1 having an amino acid sequence 100% identical to the amino acid sequence of SEQ ID NO: 251, a CDR H2 having an amino acid sequence 100% identical to the amino acid sequence of SEQ ID NO: 252, and a CDR H3 having an amino acid sequence 100% identical to the amino acid sequence of SEQ ID NO: 253; and a VL comprising therein a complementarity determining region (CDR) L1 having an amino acid sequence 100% identical to the amino acid sequence of SEQ ID NO: 255, a CDR L2 having an amino acid sequence 100% identical to the amino acid sequence of GAT, and a CDR L3 having an amino acid sequence 100% identical to the amino acid sequence of SEQ ID NO: 256. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 250 and comprising therein a complementarity determining region (CDR) H1 having an amino acid sequence 100% identical to the amino acid sequence of SEQ ID NO: 251, a CDR H2 having an amino acid sequence 100% identical to the amino acid sequence of SEQ ID NO: 252, and a CDR H3 having an amino acid sequence 100% identical to the amino acid sequence of SEQ ID NO: 253; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 254 and comprising therein a complementarity determining region (CDR) L1 having an amino acid sequence 100% identical to the amino acid sequence of SEQ ID NO: 255, a CDR L2 having an amino acid sequence 100% identical to the amino acid sequence of GAT, and a CDR L3 having an amino acid sequence 100% identical to the amino acid sequence of SEQ ID NO: 256. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 257 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 261. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 257 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 261. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 257 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 261. In some embodiments, the antibody comprises a variable heavy chain (VH) region having an amino acid sequence with at least 95% (e.g., at least 95%, 96%, 97%, 98%, 99%, or more) identity to SEQ ID NO: 257 and a variable light chain (VL) region having an amino acid sequence with at least 95% (e.g., at least 95%, 96%, 97%, 98%, 99%, or more) identity to SEQ ID NO: 261. In some embodiments, the antibody comprises a variable heavy chain (VH) region having an amino acid sequence 100% identical to the amino acid sequence of SEQ ID NO: 257 and a variable light chain (VL) region having an amino acid sequence 100% identical to the amino acid sequence of SEQ ID NO: 261. In some embodiments, the antibody comprises a VH comprising therein a complementarity determining region (CDR) H1 having an amino acid sequence 100% identical to the amino acid sequence of SEQ ID NO: 258, a CDR H2 having an amino acid sequence 100% identical to the amino acid sequence of SEQ ID NO: 259, and a CDR H3 having an amino acid sequence 100% identical to the amino acid sequence of SEQ ID NO: 260; and a VL comprising therein a complementarity determining region (CDR) L1 having an amino acid sequence 100% identical to the amino acid sequence of SEQ ID NO: 262, a CDR L2 having an amino acid sequence 100% identical to the amino acid sequence of GAS, and a CDR L3 having an amino acid sequence 100% identical to the amino acid sequence of SEQ ID NO: 263. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 257 and comprising therein a complementarity determining region (CDR) H1 having an amino acid sequence 100% identical to the amino acid sequence of SEQ ID NO: 258, a CDR H2 having an amino acid sequence 100% identical to the amino acid sequence of SEQ ID NO: 259, and a CDR H3 having an amino acid sequence 100% identical to the amino acid sequence of SEQ ID NO: 260; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 261 and comprising therein a complementarity determining region (CDR) L1 having an amino acid sequence 100% identical to the amino acid sequence of SEQ ID NO: 262, a CDR L2 having an amino acid sequence 100% identical to the amino acid sequence of GAS, and a CDR L3 having an amino acid sequence 100% identical to the amino acid sequence of SEQ ID NO: 263. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 264 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 268. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 264 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 268. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 264 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 268. In some embodiments, the antibody comprises a variable heavy chain (VH) region having an amino acid sequence with at least 95% (e.g., at least 95%, 96%, 97%, 98%, 99%, or more) identity to SEQ ID NO: 264 and a variable light chain (VL) region having an amino acid sequence with at least 95% (e.g., at least 95%, 96%, 97%, 98%, 99%, or more) identity to SEQ ID NO: 268. In some embodiments, the antibody comprises a variable heavy chain (VH) region having an amino acid sequence 100% identical to the amino acid sequence of SEQ ID NO: 264 and a variable light chain (VL) region having an amino acid sequence 100% identical to the amino acid sequence of SEQ ID NO: 268. In some embodiments, the antibody comprises a VH comprising therein a complementarity determining region (CDR) H1 having an amino acid sequence 100% identical to the amino acid sequence of SEQ ID NO: 265, a CDR H2 having an amino acid sequence 100% identical to the amino acid sequence of SEQ ID NO: 266, and a CDR H3 having an amino acid sequence 100% identical to the amino acid sequence of SEQ ID NO: 267; and a VL comprising therein a complementarity determining region (CDR) L1 having an amino acid sequence 100% identical to the amino acid sequence of SEQ ID NO: 269, a CDR L2 having an amino acid sequence 100% identical to the amino acid sequence of DAV, and a CDR L3 having an amino acid sequence 100% identical to the amino acid sequence of SEQ ID NO: 270. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 264 and comprising therein a complementarity determining region (CDR) H1 having an amino acid sequence 100% identical to the amino acid sequence of SEQ ID NO: 265, a CDR H2 having an amino acid sequence 100% identical to the amino acid sequence of SEQ ID NO: 266, and a CDR H3 having an amino acid sequence 100% identical to the amino acid sequence of SEQ ID NO: 267; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 268 and comprising therein a complementarity determining region (CDR) L1 having an amino acid sequence 100% identical to the amino acid sequence of SEQ ID NO: 269, a CDR L2 having an amino acid sequence 100% identical to the amino acid sequence of DAV, and a CDR L3 having an amino acid sequence 100% identical to the amino acid sequence of SEQ ID NO: 270In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 271 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 275. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 271 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 275. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 271 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 275. In some embodiments, the antibody comprises a variable heavy chain (VH) region having an amino acid sequence with at least 95% (e.g., at least 95%, 96%, 97%, 98%, 99%, or more) identity to SEQ ID NO: 271 and a variable light chain (VL) region having an amino acid sequence with at least 95% (e.g., at least 95%, 96%, 97%, 98%, 99%, or more) identity to SEQ ID NO: 275. In some embodiments, the antibody comprises a variable heavy chain (VH) region having an amino acid sequence 100% identical to the amino acid sequence of SEQ ID NO: 271 and a variable light chain (VL) region having an amino acid sequence 100% identical to the amino acid sequence of SEQ ID NO: 275. In some embodiments, the antibody comprises a VH comprising therein a complementarity determining region (CDR) H1 having an amino acid sequence 100% identical to the amino acid sequence of SEQ ID NO: 272, a CDR H2 having an amino acid sequence 100% identical to the amino acid sequence of SEQ ID NO: 273, and a CDR H3 having an amino acid sequence 100% identical to the amino acid sequence of SEQ ID NO: 274; and a VL comprising therein a complementarity determining region (CDR) L1 having an amino acid sequence 100% identical to the amino acid sequence of SEQ ID NO: 276, a CDR L2 having an amino acid sequence 100% identical to the amino acid sequence of DAS, and a CDR L3 having an amino acid sequence 100% identical to the amino acid sequence of SEQ ID NO: 277. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 271 and comprising therein a complementarity determining region (CDR) H1 having an amino acid sequence 100% identical to the amino acid sequence of SEQ ID NO: 272, a CDR H2 having an amino acid sequence 100% identical to the amino acid sequence of SEQ ID NO: 273, and a CDR H3 having an amino acid sequence 100% identical to the amino acid sequence of SEQ ID NO: 274; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 275 and comprising therein a complementarity determining region (CDR) L1 having an amino acid sequence 100% identical to the amino acid sequence of SEQ ID NO: 276, a CDR L2 having an amino acid sequence 100% identical to the amino acid sequence of DAS, and a CDR L3 having an amino acid sequence 100% identical to the amino acid sequence of SEQ ID NO: 277. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 278 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 282. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 278 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 282. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 278 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 282. In some embodiments, the antibody comprises a variable heavy chain (VH) region having an amino acid sequence with at least 95% (e.g., at least 95%, 96%, 97%, 98%, 99%, or more) identity to SEQ ID NO: 278 and a variable light chain (VL) region having an amino acid sequence with at least 95% (e.g., at least 95%, 96%, 97%, 98%, 99%, or more) identity to SEQ ID NO: 282. In some embodiments, the antibody comprises a variable heavy chain (VH) region having an amino acid sequence 100% identical to the amino acid sequence of SEQ ID NO: 278 and a variable light chain (VL) region having an amino acid sequence 100% identical to the amino acid sequence of SEQ ID NO: 282. In some embodiments, the antibody comprises a VH comprising therein a complementarity determining region (CDR) H1 having an amino acid sequence 100% identical to the amino acid sequence of SEQ ID NO: 279, a CDR H2 having an amino acid sequence 100% identical to the amino acid sequence of SEQ ID NO: 280, and a CDR H3 having an amino acid sequence 100% identical to the amino acid sequence of SEQ ID NO: 281; and a VL comprising therein a complementarity determining region (CDR) L1 having an amino acid sequence 100% identical to the amino acid sequence of SEQ ID NO: 283, a CDR L2 having an amino acid sequence 100% identical to the amino acid sequence of DAS, and a CDR L3 having an amino acid sequence 100% identical to the amino acid sequence of SEQ ID NO: 284. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 278 and comprising therein a complementarity determining region (CDR) H1 having an amino acid sequence 100% identical to the amino acid sequence of SEQ ID NO: 279, a CDR H2 having an amino acid sequence 100% identical to the amino acid sequence of SEQ ID NO: 280, and a CDR H3 having an amino acid sequence 100% identical to the amino acid sequence of SEQ ID NO: 281; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 282 and comprising therein a complementarity determining region (CDR) L1 having an amino acid sequence 100% identical to the amino acid sequence of SEQ ID NO: 283, a CDR L2 having an amino acid sequence 100% identical to the amino acid sequence of DAS, and a CDR L3 having an amino acid sequence 100% identical to the amino acid sequence of SEQ ID NO: 284. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 285 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 289. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 285 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 289. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 285 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 289. In some embodiments, the antibody comprises a variable heavy chain (VH) region having an amino acid sequence with at least 95% (e.g., at least 95%, 96%, 97%, 98%, 99%, or more) identity to SEQ ID NO: 285 and a variable light chain (VL) region having an amino acid sequence with at least 95% (e.g., at least 95%, 96%, 97%, 98%, 99%, or more) identity to SEQ ID NO: 289. In some embodiments, the antibody comprises a variable heavy chain (VH) region having an amino acid sequence 100% identical to the amino acid sequence of SEQ ID NO: 285 and a variable light chain (VL) region having an amino acid sequence 100% identical to the amino acid sequence of SEQ ID NO: 289. In some embodiments, the antibody comprises a VH comprising therein a complementarity determining region (CDR) H1 having an amino acid sequence 100% identical to the amino acid sequence of SEQ ID NO: 286, a CDR H2 having an amino acid sequence 100% identical to the amino acid sequence of SEQ ID NO: 287, and a CDR H3 having an amino acid sequence 100% identical to the amino acid sequence of SEQ ID NO: 288; and a VL comprising therein a complementarity determining region (CDR) L1 having an amino acid sequence 100% identical to the amino acid sequence of SEQ ID NO: 290, a CDR L2 having an amino acid sequence 100% identical to the amino acid sequence of GAS, and a CDR L3 having an amino acid sequence 100% identical to the amino acid sequence of SEQ ID NO: 291. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 285 and comprising therein a complementarity determining region (CDR) H1 having an amino acid sequence 100% identical to the amino acid sequence of SEQ ID NO: 286, a CDR H2 having an amino acid sequence 100% identical to the amino acid sequence of SEQ ID NO: 287, and a CDR H3 having an amino acid sequence 100% identical to the amino acid sequence of SEQ ID NO: 288; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 289 and comprising therein a complementarity determining region (CDR) L1 having an amino acid sequence 100% identical to the amino acid sequence of SEQ ID NO: 290, a CDR L2 having an amino acid sequence 100% identical to the amino acid sequence of GAS, and a CDR L3 having an amino acid sequence 100% identical to the amino acid sequence of SEQ ID NO: 291. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 292 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 296. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 292 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 296. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 292 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 296. In some embodiments, the antibody comprises a variable heavy chain (VH) region having an amino acid sequence with at least 95% (e.g., at least 95%, 96%, 97%, 98%, 99%, or more) identity to SEQ ID NO: 292 and a variable light chain (VL) region having an amino acid sequence with at least 95% (e.g., at least 95%, 96%, 97%, 98%, 99%, or more) identity to SEQ ID NO: 296. In some embodiments, the antibody comprises a variable heavy chain (VH) region having an amino acid sequence 100% identical to the amino acid sequence of SEQ ID NO: 292 and a variable light chain (VL) region having an amino acid sequence 100% identical to the amino acid sequence of SEQ ID NO: 296. In some embodiments, the antibody comprises a VH comprising therein a complementarity determining region (CDR) H1 having an amino acid sequence 100% identical to the amino acid sequence of SEQ ID NO: 293, a CDR H2 having an amino acid sequence 100% identical to the amino acid sequence of SEQ ID NO: 294, and a CDR H3 having an amino acid sequence 100% identical to the amino acid sequence of SEQ ID NO: 295; and a VL comprising therein a complementarity determining region (CDR) L1 having an amino acid sequence 100% identical to the amino acid sequence of SEQ ID NO: 297, a CDR L2 having an amino acid sequence 100% identical to the amino acid sequence of KAS, and a CDR L3 having an amino acid sequence 100% identical to the amino acid sequence of SEQ ID NO: 298. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 292 and comprising therein a complementarity determining region (CDR) H1 having an amino acid sequence 100% identical to the amino acid sequence of SEQ ID NO: 293, a CDR H2 having an amino acid sequence 100% identical to the amino acid sequence of SEQ ID NO: 294, and a CDR H3 having an amino acid sequence 100% identical to the amino acid sequence of SEQ ID NO: 295; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 296 and comprising therein a complementarity determining region (CDR) L1 having an amino acid sequence 100% identical to the amino acid sequence of SEQ ID NO: 297, a CDR L2 having an amino acid sequence 100% identical to the amino acid sequence of KAS, and a CDR L3 having an amino acid sequence 100% identical to the amino acid sequence of SEQ ID NO: 298. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 299 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 303. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 299 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 303. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 299 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 303. In some embodiments, the antibody comprises a variable heavy chain (VH) region having an amino acid sequence with at least 95% (e.g., at least 95%, 96%, 97%, 98%, 99%, or more) identity to SEQ ID NO: 299 and a variable light chain (VL) region having an amino acid sequence with at least 95% (e.g., at least 95%, 96%, 97%, 98%, 99%, or more) identity to SEQ ID NO: 303. In some embodiments, the antibody comprises a variable heavy chain (VH) region having an amino acid sequence 100% identical to the amino acid sequence of SEQ ID NO: 299 and a variable light chain (VL) region having an amino acid sequence 100% identical to the amino acid sequence of SEQ ID NO: 303. In some embodiments, the antibody comprises a VH comprising therein a complementarity determining region (CDR) H1 having an amino acid sequence 100% identical to the amino acid sequence of SEQ ID NO: 300, a CDR H2 having an amino acid sequence 100% identical to the amino acid sequence of SEQ ID NO: 301, and a CDR H3 having an amino acid sequence 100% identical to the amino acid sequence of SEQ ID NO: 302; and a VL comprising therein a complementarity determining region (CDR) L1 having an amino acid sequence 100% identical to the amino acid sequence of SEQ ID NO: 304, a CDR L2 having an amino acid sequence 100% identical to the amino acid sequence of AAS, and a CDR L3 having an amino acid sequence 100% identical to the amino acid sequence of SEQ ID NO: 305. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 299 and comprising therein a complementarity determining region (CDR) H1 having an amino acid sequence 100% identical to the amino acid sequence of SEQ ID NO: 300, a CDR H2 having an amino acid sequence 100% identical to the amino acid sequence of SEQ ID NO: 301, and a CDR H3 having an amino acid sequence 100% identical to the amino acid sequence of SEQ ID NO: 302; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 303 and comprising therein a complementarity determining region (CDR) L1 having an amino acid sequence 100% identical to the amino acid sequence of SEQ ID NO: 304, a CDR L2 having an amino acid sequence 100% identical to the amino acid sequence of AAS, and a CDR L3 having an amino acid sequence 100% identical to the amino acid sequence of SEQ ID NO: 305. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 306 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 310. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 306 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 310. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 306 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 310. In some embodiments, the antibody comprises a variable heavy chain (VH) region having an amino acid sequence with at least 95% (e.g., at least 95%, 96%, 97%, 98%, 99%, or more) identity to SEQ ID NO: 306 and a variable light chain (VL) region having an amino acid sequence with at least 95% (e.g., at least 95%, 96%, 97%, 98%, 99%, or more) identity to SEQ ID NO: 310. In some embodiments, the antibody comprises a variable heavy chain (VH) region having an amino acid sequence 100% identical to the amino acid sequence of SEQ ID NO: 306 and a variable light chain (VL) region having an amino acid sequence 100% identical to the amino acid sequence of SEQ ID NO: 310. In some embodiments, the antibody comprises a VH comprising therein a complementarity determining region (CDR) H1 having an amino acid sequence 100% identical to the amino acid sequence of SEQ ID NO: 307, a CDR H2 having an amino acid sequence 100% identical to the amino acid sequence of SEQ ID NO: 308, and a CDR H3 having an amino acid sequence 100% identical to the amino acid sequence of SEQ ID NO: 309; and a VL comprising therein a complementarity determining region (CDR) L1 having an amino acid sequence 100% identical to the amino acid sequence of SEQ ID NO: 311, a CDR L2 having an amino acid sequence 100% identical to the amino acid sequence of DTS, and a CDR L3 having an amino acid sequence 100% identical to the amino acid sequence of SEQ ID NO: 312. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 306 and comprising therein a complementarity determining region (CDR) H1 having an amino acid sequence 100% identical to the amino acid sequence of SEQ ID NO: 307, a CDR H2 having an amino acid sequence 100% identical to the amino acid sequence of SEQ ID NO: 308, and a CDR H3 having an amino acid sequence 100% identical to the amino acid sequence of SEQ ID NO: 309; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 310 and comprising therein a complementarity determining region (CDR) L1 having an amino acid sequence 100% identical to the amino acid sequence of SEQ ID NO: 311, a CDR L2 having an amino acid sequence 100% identical to the amino acid sequence of DTS, and a CDR L3 having an amino acid sequence 100% identical to the amino acid sequence of SEQ ID NO: 312. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 461, a second heavy chain region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 481, and a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 471. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 313 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 317. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 313 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 317. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 313 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 317. In some embodiments, the antibody comprises a variable heavy chain (VH) region having an amino acid sequence with at least 95% (e.g., at least 95%, 96%, 97%, 98%, 99%, or more) identity to SEQ ID NO: 313 and a variable light chain (VL) region having an amino acid sequence with at least 95% (e.g., at least 95%, 96%, 97%, 98%, 99%, or more) identity to SEQ ID NO: 317. In some embodiments, the antibody comprises a variable heavy chain (VH) region having an amino acid sequence 100% identical to the amino acid sequence of SEQ ID NO: 313 and a variable light chain (VL) region having an amino acid sequence 100% identical to the amino acid sequence of SEQ ID NO: 317. In some embodiments, the antibody comprises a VH comprising therein a complementarity determining region (CDR) H1 having an amino acid sequence 100% identical to the amino acid sequence of SEQ ID NO: 314, a CDR H2 having an amino acid sequence 100% identical to the amino acid sequence of SEQ ID NO: 315, and a CDR H3 having an amino acid sequence 100% identical to the amino acid sequence of SEQ ID NO: 316; and a VL comprising therein a complementarity determining region (CDR) L1 having an amino acid sequence 100% identical to the amino acid sequence of SEQ ID NO: 318, a CDR L2 having an amino acid sequence 100% identical to the amino acid sequence of DAS, and a CDR L3 having an amino acid sequence 100% identical to the amino acid sequence of SEQ ID NO: 319. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 313 and comprising therein a complementarity determining region (CDR) H1 having an amino acid sequence 100% identical to the amino acid sequence of SEQ ID NO: 314, a CDR H2 having an amino acid sequence 100% identical to the amino acid sequence of SEQ ID NO: 315, and a CDR H3 having an amino acid sequence 100% identical to the amino acid sequence of SEQ ID NO: 316; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 317 and comprising therein a complementarity determining region (CDR) L1 having an amino acid sequence 100% identical to the amino acid sequence of SEQ ID NO: 318, a CDR L2 having an amino acid sequence 100% identical to the amino acid sequence of DAS, and a CDR L3 having an amino acid sequence 100% identical to the amino acid sequence of SEQ ID NO: 319. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 320 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 324. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 320 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 324. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 320 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 324. In some embodiments, the antibody comprises a variable heavy chain (VH) region having an amino acid sequence with at least 95% (e.g., at least 95%, 96%, 97%, 98%, 99%, or more) identity to SEQ ID NO: 320 and a variable light chain (VL) region having an amino acid sequence with at least 95% (e.g., at least 95%, 96%, 97%, 98%, 99%, or more) identity to SEQ ID NO: 324. In some embodiments, the antibody comprises a variable heavy chain (VH) region having an amino acid sequence 100% identical to the amino acid sequence of SEQ ID NO: 320 and a variable light chain (VL) region having an amino acid sequence 100% identical to the amino acid sequence of SEQ ID NO: 324. In some embodiments, the antibody comprises a VH comprising therein a complementarity determining region (CDR) H1 having an amino acid sequence 100% identical to the amino acid sequence of SEQ ID NO: 321, a CDR H2 having an amino acid sequence 100% identical to the amino acid sequence of SEQ ID NO: 322, and a CDR H3 having an amino acid sequence 100% identical to the amino acid sequence of SEQ ID NO: 323; and a VL comprising therein a complementarity determining region (CDR) L1 having an amino acid sequence 100% identical to the amino acid sequence of SEQ ID NO: 325, a CDR L2 having an amino acid sequence 100% identical to the amino acid sequence of STS, and a CDR L3 having an amino acid sequence 100% identical to the amino acid sequence of SEQ ID NO: 326. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 320 and comprising therein a complementarity determining region (CDR) H1 having an amino acid sequence 100% identical to the amino acid sequence of SEQ ID NO: 321, a CDR H2 having an amino acid sequence 100% identical to the amino acid sequence of SEQ ID NO: 322, and a CDR H3 having an amino acid sequence 100% identical to the amino acid sequence of SEQ ID NO: 323; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 324 and comprising therein a complementarity determining region (CDR) L1 having an amino acid sequence 100% identical to the amino acid sequence of SEQ ID NO: 325, a CDR L2 having an amino acid sequence 100% identical to the amino acid sequence of STS, and a CDR L3 having an amino acid sequence 100% identical to the amino acid sequence of SEQ ID NO: 326. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 327 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 331. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 327 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 331. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 327 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 331. In some embodiments, the antibody comprises a variable heavy chain (VH) region having an amino acid sequence with at least 95% (e.g., at least 95%, 96%, 97%, 98%, 99%, or more) identity to SEQ ID NO: 327 and a variable light chain (VL) region having an amino acid sequence with at least 95% (e.g., at least 95%, 96%, 97%, 98%, 99%, or more) identity to SEQ ID NO: 331. In some embodiments, the antibody comprises a variable heavy chain (VH) region having an amino acid sequence 100% identical to the amino acid sequence of SEQ ID NO: 327 and a variable light chain (VL) region having an amino acid sequence 100% identical to the amino acid sequence of SEQ ID NO: 331. In some embodiments, the antibody comprises a VH comprising therein a complementarity determining region (CDR) H1 having an amino acid sequence 100% identical to the amino acid sequence of SEQ ID NO: 328, a CDR H2 having an amino acid sequence 100% identical to the amino acid sequence of SEQ ID NO: 329, and a CDR H3 having an amino acid sequence 100% identical to the amino acid sequence of SEQ ID NO: 330; and a VL comprising therein a complementarity determining region (CDR) L1 having an amino acid sequence 100% identical to the amino acid sequence of SEQ ID NO: 332, a CDR L2 having an amino acid sequence 100% identical to the amino acid sequence of DAS, and a CDR L3 having an amino acid sequence 100% identical to the amino acid sequence of SEQ ID NO: 333. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 327 and comprising therein a complementarity determining region (CDR) H1 having an amino acid sequence 100% identical to the amino acid sequence of SEQ ID NO: 328, a CDR H2 having an amino acid sequence 100% identical to the amino acid sequence of SEQ ID NO: 329, and a CDR H3 having an amino acid sequence 100% identical to the amino acid sequence of SEQ ID NO: 330; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 331 and comprising therein a complementarity determining region (CDR) L1 having an amino acid sequence 100% identical to the amino acid sequence of SEQ ID NO: 332, a CDR L2 having an amino acid sequence 100% identical to the amino acid sequence of DAS, and a CDR L3 having an amino acid sequence 100% identical to the amino acid sequence of SEQ ID NO: 333. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 334 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 338. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 334 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 338. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 334 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 338. In some embodiments, the antibody comprises a variable heavy chain (VH) region having an amino acid sequence with at least 95% (e.g., at least 95%, 96%, 97%, 98%, 99%, or more) identity to SEQ ID NO: 334 and a variable light chain (VL) region having an amino acid sequence with at least 95% (e.g., at least 95%, 96%, 97%, 98%, 99%, or more) identity to SEQ ID NO: 338. In some embodiments, the antibody comprises a variable heavy chain (VH) region having an amino acid sequence 100% identical to the amino acid sequence of SEQ ID NO: 334 and a variable light chain (VL) region having an amino acid sequence 100% identical to the amino acid sequence of SEQ ID NO: 338. In some embodiments, the antibody comprises a VH comprising therein a complementarity determining region (CDR) H1 having an amino acid sequence 100% identical to the amino acid sequence of SEQ ID NO: 335, a CDR H2 having an amino acid sequence 100% identical to the amino acid sequence of SEQ ID NO: 336, and a CDR H3 having an amino acid sequence 100% identical to the amino acid sequence of SEQ ID NO: 337; and a VL comprising therein a complementarity determining region (CDR) L1 having an amino acid sequence 100% identical to the amino acid sequence of SEQ ID NO: 339, a CDR L2 having an amino acid sequence 100% identical to the amino acid sequence of GAS, and a CDR L3 having an amino acid sequence 100% identical to the amino acid sequence of SEQ ID NO: 340. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 334 and comprising therein a complementarity determining region (CDR) H1 having an amino acid sequence 100% identical to the amino acid sequence of SEQ ID NO: 335, a CDR H2 having an amino acid sequence 100% identical to the amino acid sequence of SEQ ID NO: 336, and a CDR H3 having an amino acid sequence 100% identical to the amino acid sequence of SEQ ID NO: 337; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 338 and comprising therein a complementarity determining region (CDR) L1 having an amino acid sequence 100% identical to the amino acid sequence of SEQ ID NO: 339, a CDR L2 having an amino acid sequence 100% identical to the amino acid sequence of GAS, and a CDR L3 having an amino acid sequence 100% identical to the amino acid sequence of SEQ ID NO: 340. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 341 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 345. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 341 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 345. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 341 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 345. In some embodiments, the antibody comprises a variable heavy chain (VH) region having an amino acid sequence with at least 95% (e.g., at least 95%, 96%, 97%, 98%, 99%, or more) identity to SEQ ID NO: 341 and a variable light chain (VL) region having an amino acid sequence with at least 95% (e.g., at least 95%, 96%, 97%, 98%, 99%, or more) identity to SEQ ID NO: 345. In some embodiments, the antibody comprises a variable heavy chain (VH) region having an amino acid sequence 100% identical to the amino acid sequence of SEQ ID NO: 341 and a variable light chain (VL) region having an amino acid sequence 100% identical to the amino acid sequence of SEQ ID NO: 345. In some embodiments, the antibody comprises a VH comprising therein a complementarity determining region (CDR) H1 having an amino acid sequence 100% identical to the amino acid sequence of SEQ ID NO: 342, a CDR H2 having an amino acid sequence 100% identical to the amino acid sequence of SEQ ID NO: 343, and a CDR H3 having an amino acid sequence 100% identical to the amino acid sequence of SEQ ID NO: 344; and a VL comprising therein a complementarity determining region (CDR) L1 having an amino acid sequence 100% identical to the amino acid sequence of SEQ ID NO: 346, a CDR L2 having an amino acid sequence 100% identical to the amino acid sequence of DAS, and a CDR L3 having an amino acid sequence 100% identical to the amino acid sequence of SEQ ID NO: 347. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 341 and comprising therein a complementarity determining region (CDR) H1 having an amino acid sequence 100% identical to the amino acid sequence of SEQ ID NO: 342, a CDR H2 having an amino acid sequence 100% identical to the amino acid sequence of SEQ ID NO: 343, and a CDR H3 having an amino acid sequence 100% identical to the amino acid sequence of SEQ ID NO: 344; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 345 and comprising therein a complementarity determining region (CDR) L1 having an amino acid sequence 100% identical to the amino acid sequence of SEQ ID NO: 346, a CDR L2 having an amino acid sequence 100% identical to the amino acid sequence of DAS, and a CDR L3 having an amino acid sequence 100% identical to the amino acid sequence of SEQ ID NO: 347. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 348 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 352. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 348 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 352. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 348 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 352. In some embodiments, the antibody comprises a variable heavy chain (VH) region having an amino acid sequence with at least 95% (e.g., at least 95%, 96%, 97%, 98%, 99%, or more) identity to SEQ ID NO: 348 and a variable light chain (VL) region having an amino acid sequence with at least 95% (e.g., at least 95%, 96%, 97%, 98%, 99%, or more) identity to SEQ ID NO: 352. In some embodiments, the antibody comprises a variable heavy chain (VH) region having an amino acid sequence 100% identical to the amino acid sequence of SEQ ID NO: 348 and a variable light chain (VL) region having an amino acid sequence 100% identical to the amino acid sequence of SEQ ID NO: 352. In some embodiments, the antibody comprises a VH comprising therein a complementarity determining region (CDR) H1 having an amino acid sequence 100% identical to the amino acid sequence of SEQ ID NO: 349, a CDR H2 having an amino acid sequence 100% identical to the amino acid sequence of SEQ ID NO: 350, and a CDR H3 having an amino acid sequence 100% identical to the amino acid sequence of SEQ ID NO: 351; and a VL comprising therein a complementarity determining region (CDR) L1 having an amino acid sequence 100% identical to the amino acid sequence of SEQ ID NO: 353, a CDR L2 having an amino acid sequence 100% identical to the amino acid sequence of DTA, and a CDR L3 having an amino acid sequence 100% identical to the amino acid sequence of SEQ ID NO: 354. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 348 and comprising therein a complementarity determining region (CDR) H1 having an amino acid sequence 100% identical to the amino acid sequence of SEQ ID NO: 349, a CDR H2 having an amino acid sequence 100% identical to the amino acid sequence of SEQ ID NO: 350, and a CDR H3 having an amino acid sequence 100% identical to the amino acid sequence of SEQ ID NO: 351; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 352 and comprising therein a complementarity determining region (CDR) L1 having an amino acid sequence 100% identical to the amino acid sequence of SEQ ID NO: 353, a CDR L2 having an amino acid sequence 100% identical to the amino acid sequence of DTS, and a CDR L3 having an amino acid sequence 100% identical to the amino acid sequence of SEQ ID NO: 354. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 355 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 359. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 355 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 359. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 355 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 359. In some embodiments, the antibody comprises a variable heavy chain (VH) region having an amino acid sequence with at least 95% (e.g., at least 95%, 96%, 97%, 98%, 99%, or more) identity to SEQ ID NO: 355 and a variable light chain (VL) region having an amino acid sequence with at least 95% (e.g., at least 95%, 96%, 97%, 98%, 99%, or more) identity to SEQ ID NO: 359. In some embodiments, the antibody comprises a variable heavy chain (VH) region having an amino acid sequence 100% identical to the amino acid sequence of SEQ ID NO: 355 and a variable light chain (VL) region having an amino acid sequence 100% identical to the amino acid sequence of SEQ ID NO: 359. In some embodiments, the antibody comprises a VH comprising therein a complementarity determining region (CDR) H1 having an amino acid sequence 100% identical to the amino acid sequence of SEQ ID NO: 356, a CDR H2 having an amino acid sequence 100% identical to the amino acid sequence of SEQ ID NO: 357, and a CDR H3 having an amino acid sequence 100% identical to the amino acid sequence of SEQ ID NO: 358; and a VL comprising therein a complementarity determining region (CDR) L1 having an amino acid sequence 100% identical to the amino acid sequence of SEQ ID NO: 360, a CDR L2 having an amino acid sequence 100% identical to the amino acid sequence of AAS, and a CDR L3 having an amino acid sequence 100% identical to the amino acid sequence of SEQ ID NO: 361. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 355 and comprising therein a complementarity determining region (CDR) H1 having an amino acid sequence 100% identical to the amino acid sequence of SEQ ID NO: 356, a CDR H2 having an amino acid sequence 100% identical to the amino acid sequence of SEQ ID NO: 357, and a CDR H3 having an amino acid sequence 100% identical to the amino acid sequence of SEQ ID NO: 358; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 359 and comprising therein a complementarity determining region (CDR) L1 having an amino acid sequence 100% identical to the amino acid sequence of SEQ ID NO: 360, a CDR L2 having an amino acid sequence 100% identical to the amino acid sequence of AAS, and a CDR L3 having an amino acid sequence 100% identical to the amino acid sequence of SEQ ID NO: 361. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 362 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 366. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 362 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 366. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 362 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 366. In some embodiments, the antibody comprises a variable heavy chain (VH) region having an amino acid sequence with at least 95% (e.g., at least 95%, 96%, 97%, 98%, 99%, or more) identity to SEQ ID NO: 362 and a variable light chain (VL) region having an amino acid sequence with at least 95% (e.g., at least 95%, 96%, 97%, 98%, 99%, or more) identity to SEQ ID NO: 366. In some embodiments, the antibody comprises a variable heavy chain (VH) region having an amino acid sequence 100% identical to the amino acid sequence of SEQ ID NO: 362 and a variable light chain (VL) region having an amino acid sequence 100% identical to the amino acid sequence of SEQ ID NO: 366. In some embodiments, the antibody comprises a VH comprising therein a complementarity determining region (CDR) H1 having an amino acid sequence 100% identical to the amino acid sequence of SEQ ID NO: 363, a CDR H2 having an amino acid sequence 100% identical to the amino acid sequence of SEQ ID NO: 364, and a CDR H3 having an amino acid sequence 100% identical to the amino acid sequence of SEQ ID NO: 365; and a VL comprising therein a complementarity determining region (CDR) L1 having an amino acid sequence 100% identical to the amino acid sequence of SEQ ID NO: 367, a CDR L2 having an amino acid sequence 100% identical to the amino acid sequence of AAS, and a CDR L3 having an amino acid sequence 100% identical to the amino acid sequence of SEQ ID NO: 368. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 362 and comprising therein a complementarity determining region (CDR) H1 having an amino acid sequence 100% identical to the amino acid sequence of SEQ ID NO: 363, a CDR H2 having an amino acid sequence 100% identical to the amino acid sequence of SEQ ID NO: 364, and a CDR H3 having an amino acid sequence 100% identical to the amino acid sequence of SEQ ID NO: 365; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 366 and comprising therein a complementarity determining region (CDR) L1 having an amino acid sequence 100% identical to the amino acid sequence of SEQ ID NO: 367, a CDR L2 having an amino acid sequence 100% identical to the amino acid sequence of AAS, and a CDR L3 having an amino acid sequence 100% identical to the amino acid sequence of SEQ ID NO: 368. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 369 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 373. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 369 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 373. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 369 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 373. In some embodiments, the antibody comprises a variable heavy chain (VH) region having an amino acid sequence with at least 95% (e.g., at least 95%, 96%, 97%, 98%, 99%, or more) identity to SEQ ID NO: 369 and a variable light chain (VL) region having an amino acid sequence with at least 95% (e.g., at least 95%, 96%, 97%, 98%, 99%, or more) identity to SEQ ID NO: 373. In some embodiments, the antibody comprises a variable heavy chain (VH) region having an amino acid sequence 100% identical to the amino acid sequence of SEQ ID NO: 369 and a variable light chain (VL) region having an amino acid sequence 100% identical to the amino acid sequence of SEQ ID NO: 373. In some embodiments, the antibody comprises a VH comprising therein a complementarity determining region (CDR) H1 having an amino acid sequence 100% identical to the amino acid sequence of SEQ ID NO: 370, a CDR H2 having an amino acid sequence 100% identical to the amino acid sequence of SEQ ID NO: 371, and a CDR H3 having an amino acid sequence 100% identical to the amino acid sequence of SEQ ID NO: 372; and a VL comprising therein a complementarity determining region (CDR) L1 having an amino acid sequence 100% identical to the amino acid sequence of SEQ ID NO: 374, a CDR L2 having an amino acid sequence 100% identical to the amino acid sequence of GTS, and a CDR L3 having an amino acid sequence 100% identical to the amino acid sequence of SEQ ID NO: 375. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 369 and comprising therein a complementarity determining region (CDR) H1 having an amino acid sequence 100% identical to the amino acid sequence of SEQ ID NO: 370, a CDR H2 having an amino acid sequence 100% identical to the amino acid sequence of SEQ ID NO: 371, and a CDR H3 having an amino acid sequence 100% identical to the amino acid sequence of SEQ ID NO: 372; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 373 and comprising therein a complementarity determining region (CDR) L1 having an amino acid sequence 100% identical to the amino acid sequence of SEQ ID NO: 374, a CDR L2 having an amino acid sequence 100% identical to the amino acid sequence of GTS, and a CDR L3 having an amino acid sequence 100% identical to the amino acid sequence of SEQ ID NO: 375. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 376 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 380. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 376 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 380. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 376 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 380. In some embodiments, the antibody comprises a variable heavy chain (VH) region having an amino acid sequence with at least 95% (e.g., at least 95%, 96%, 97%, 98%, 99%, or more) identity to SEQ ID NO: 376 and a variable light chain (VL) region having an amino acid sequence with at least 95% (e.g., at least 95%, 96%, 97%, 98%, 99%, or more) identity to SEQ ID NO: 380. In some embodiments, the antibody comprises a variable heavy chain (VH) region having an amino acid sequence 100% identical to the amino acid sequence of SEQ ID NO: 376 and a variable light chain (VL) region having an amino acid sequence 100% identical to the amino acid sequence of SEQ ID NO: 380. In some embodiments, the antibody comprises a VH comprising therein a complementarity determining region (CDR) H1 having an amino acid sequence 100% identical to the amino acid sequence of SEQ ID NO: 377, a CDR H2 having an amino acid sequence 100% identical to the amino acid sequence of SEQ ID NO: 378, and a CDR H3 having an amino acid sequence 100% identical to the amino acid sequence of SEQ ID NO: 379; and a VL comprising therein a complementarity determining region (CDR) L1 having an amino acid sequence 100% identical to the amino acid sequence of SEQ ID NO: 381, a CDR L2 having an amino acid sequence 100% identical to the amino acid sequence of ETS, and a CDR L3 having an amino acid sequence 100% identical to the amino acid sequence of SEQ ID NO: 382. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 376 and comprising therein a complementarity determining region (CDR) H1 having an amino acid sequence 100% identical to the amino acid sequence of SEQ ID NO: 377, a CDR H2 having an amino acid sequence 100% identical to the amino acid sequence of SEQ ID NO: 378, and a CDR H3 having an amino acid sequence 100% identical to the amino acid sequence of SEQ ID NO: 379; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 380 and comprising therein a complementarity determining region (CDR) L1 having an amino acid sequence 100% identical to the amino acid sequence of SEQ ID NO: 381, a CDR L2 having an amino acid sequence 100% identical to the amino acid sequence of ETS, and a CDR L3 having an amino acid sequence 100% identical to the amino acid sequence of SEQ ID NO: 382. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 383 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 387. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 383 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 387. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 383 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 387. In some embodiments, the antibody comprises a variable heavy chain (VH) region having an amino acid sequence with at least 95% (e.g., at least 95%, 96%, 97%, 98%, 99%, or more) identity to SEQ ID NO: 383 and a variable light chain (VL) region having an amino acid sequence with at least 95% (e.g., at least 95%, 96%, 97%, 98%, 99%, or more) identity to SEQ ID NO: 387. In some embodiments, the antibody comprises a variable heavy chain (VH) region having an amino acid sequence 100% identical to the amino acid sequence of SEQ ID NO: 383 and a variable light chain (VL) region having an amino acid sequence 100% identical to the amino acid sequence of SEQ ID NO: 387. In some embodiments, the antibody comprises a VH comprising therein a complementarity determining region (CDR) H1 having an amino acid sequence 100% identical to the amino acid sequence of SEQ ID NO: 384, a CDR H2 having an amino acid sequence 100% identical to the amino acid sequence of SEQ ID NO: 385, and a CDR H3 having an amino acid sequence 100% identical to the amino acid sequence of SEQ ID NO: 386; and a VL comprising therein a complementarity determining region (CDR) L1 having an amino acid sequence 100% identical to the amino acid sequence of SEQ ID NO: 388, a CDR L2 having an amino acid sequence 100% identical to the amino acid sequence of GAS, and a CDR L3 having an amino acid sequence 100% identical to the amino acid sequence of SEQ ID NO: 389. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 383 and comprising therein a complementarity determining region (CDR) H1 having an amino acid sequence 100% identical to the amino acid sequence of SEQ ID NO: 384, a CDR H2 having an amino acid sequence 100% identical to the amino acid sequence of SEQ ID NO: 385, and a CDR H3 having an amino acid sequence 100% identical to the amino acid sequence of SEQ ID NO: 386; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 387 and comprising therein a complementarity determining region (CDR) L1 having an amino acid sequence 100% identical to the amino acid sequence of SEQ ID NO: 388, a CDR L2 having an amino acid sequence 100% identical to the amino acid sequence of GAS, and a CDR L3 having an amino acid sequence 100% identical to the amino acid sequence of SEQ ID NO: 389. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 390 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 394. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 390 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 394. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 390 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 394. In some embodiments, the antibody comprises a variable heavy chain (VH) region having an amino acid sequence with at least 95% (e.g., at least 95%, 96%, 97%, 98%, 99%, or more) identity to SEQ ID NO: 390 and a variable light chain (VL) region having an amino acid sequence with at least 95% (e.g., at least 95%, 96%, 97%, 98%, 99%, or more) identity to SEQ ID NO: 394. In some embodiments, the antibody comprises a variable heavy chain (VH) region having an amino acid sequence 100% identical to the amino acid sequence of SEQ ID NO: 390 and a variable light chain (VL) region having an amino acid sequence 100% identical to the amino acid sequence of SEQ ID NO: 394. In some embodiments, the antibody comprises a VH comprising therein a complementarity determining region (CDR) H1 having an amino acid sequence 100% identical to the amino acid sequence of SEQ ID NO: 391, a CDR H2 having an amino acid sequence 100% identical to the amino acid sequence of SEQ ID NO: 392, and a CDR H3 having an amino acid sequence 100% identical to the amino acid sequence of SEQ ID NO: 393; and a VL comprising therein a complementarity determining region (CDR) L1 having an amino acid sequence 100% identical to the amino acid sequence of SEQ ID NO: 395, a CDR L2 having an amino acid sequence 100% identical to the amino acid sequence of DAS, and a CDR L3 having an amino acid sequence 100% identical to the amino acid sequence of SEQ ID NO: 396. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 390 and comprising therein a complementarity determining region (CDR) H1 having an amino acid sequence 100% identical to the amino acid sequence of SEQ ID NO: 391, a CDR H2 having an amino acid sequence 100% identical to the amino acid sequence of SEQ ID NO: 392, and a CDR H3 having an amino acid sequence 100% identical to the amino acid sequence of SEQ ID NO: 393; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 394 and comprising therein a complementarity determining region (CDR) L1 having an amino acid sequence 100% identical to the amino acid sequence of SEQ ID NO: 395, a CDR L2 having an amino acid sequence 100% identical to the amino acid sequence of DAS, and a CDR L3 having an amino acid sequence 100% identical to the amino acid sequence of SEQ ID NO: 396. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 462, a second heavy chain region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 482, and a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 472. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 397 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 401. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 397 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 401. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 397 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 401. In some embodiments, the antibody comprises a variable heavy chain (VH) region having an amino acid sequence with at least 95% (e.g., at least 95%, 96%, 97%, 98%, 99%, or more) identity to SEQ ID NO: 397 and a variable light chain (VL) region having an amino acid sequence with at least 95% (e.g., at least 95%, 96%, 97%, 98%, 99%, or more) identity to SEQ ID NO: 401. In some embodiments, the antibody comprises a variable heavy chain (VH) region having an amino acid sequence 100% identical to the amino acid sequence of SEQ ID NO: 397 and a variable light chain (VL) region having an amino acid sequence 100% identical to the amino acid sequence of SEQ ID NO: 401. In some embodiments, the antibody comprises a VH comprising therein a complementarity determining region (CDR) H1 having an amino acid sequence 100% identical to the amino acid sequence of SEQ ID NO: 398, a CDR H2 having an amino acid sequence 100% identical to the amino acid sequence of SEQ ID NO: 399, and a CDR H3 having an amino acid sequence 100% identical to the amino acid sequence of SEQ ID NO: 400; and a VL comprising therein a complementarity determining region (CDR) L1 having an amino acid sequence 100% identical to the amino acid sequence of SEQ ID NO: 402, a CDR L2 having an amino acid sequence 100% identical to the amino acid sequence of GAS, and a CDR L3 having an amino acid sequence 100% identical to the amino acid sequence of SEQ ID NO: 403. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 397 and comprising therein a complementarity determining region (CDR) H1 having an amino acid sequence 100% identical to the amino acid sequence of SEQ ID NO: 398, a CDR H2 having an amino acid sequence 100% identical to the amino acid sequence of SEQ ID NO: 399, and a CDR H3 having an amino acid sequence 100% identical to the amino acid sequence of SEQ ID NO: 400; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 401 and comprising therein a complementarity determining region (CDR) L1 having an amino acid sequence 100% identical to the amino acid sequence of SEQ ID NO: 402, a CDR L2 having an amino acid sequence 100% identical to the amino acid sequence of GAS, and a CDR L3 having an amino acid sequence 100% identical to the amino acid sequence of SEQ ID NO: 403. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 404 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 408. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 404 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 408. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 404 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 408. In some embodiments, the antibody comprises a variable heavy chain (VH) region having an amino acid sequence with at least 95% (e.g., at least 95%, 96%, 97%, 98%, 99%, or more) identity to SEQ ID NO: 404 and a variable light chain (VL) region having an amino acid sequence with at least 95% (e.g., at least 95%, 96%, 97%, 98%, 99%, or more) identity to SEQ ID NO: 408. In some embodiments, the antibody comprises a variable heavy chain (VH) region having an amino acid sequence 100% identical to the amino acid sequence of SEQ ID NO: 404 and a variable light chain (VL) region having an amino acid sequence 100% identical to the amino acid sequence of SEQ ID NO: 408. In some embodiments, the antibody comprises a VH comprising therein a complementarity determining region (CDR) H1 having an amino acid sequence 100% identical to the amino acid sequence of SEQ ID NO: 405, a CDR H2 having an amino acid sequence 100% identical to the amino acid sequence of SEQ ID NO: 406, and a CDR H3 having an amino acid sequence 100% identical to the amino acid sequence of SEQ ID NO: 407; and a VL comprising therein a complementarity determining region (CDR) L1 having an amino acid sequence 100% identical to the amino acid sequence of SEQ ID NO: 409, a CDR L2 having an amino acid sequence 100% identical to the amino acid sequence of DAS, and a CDR L3 having an amino acid sequence 100% identical to the amino acid sequence of SEQ ID NO: 410. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 404 and comprising therein a complementarity determining region (CDR) H1 having an amino acid sequence 100% identical to the amino acid sequence of SEQ ID NO: 405, a CDR H2 having an amino acid sequence 100% identical to the amino acid sequence of SEQ ID NO: 406, and a CDR H3 having an amino acid sequence 100% identical to the amino acid sequence of SEQ ID NO: 407; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 408 and comprising therein a complementarity determining region (CDR) L1 having an amino acid sequence 100% identical to the amino acid sequence of SEQ ID NO: 409, a CDR L2 having an amino acid sequence 100% identical to the amino acid sequence of DAS, and a CDR L3 having an amino acid sequence 100% identical to the amino acid sequence of SEQ ID NO: 410. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 411 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 415. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 411 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 415. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 411 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 415. In some embodiments, the antibody comprises a variable heavy chain (VH) region having an amino acid sequence with at least 95% (e.g., at least 95%, 96%, 97%, 98%, 99%, or more) identity to SEQ ID NO: 411 and a variable light chain (VL) region having an amino acid sequence with at least 95% (e.g., at least 95%, 96%, 97%, 98%, 99%, or more) identity to SEQ ID NO: 415. In some embodiments, the antibody comprises a variable heavy chain (VH) region having an amino acid sequence 100% identical to the amino acid sequence of SEQ ID NO: 411 and a variable light chain (VL) region having an amino acid sequence 100% identical to the amino acid sequence of SEQ ID NO: 415. In some embodiments, the antibody comprises a VH comprising therein a complementarity determining region (CDR) H1 having an amino acid sequence 100% identical to the amino acid sequence of SEQ ID NO: 412, a CDR H2 having an amino acid sequence 100% identical to the amino acid sequence of SEQ ID NO: 413, and a CDR H3 having an amino acid sequence 100% identical to the amino acid sequence of SEQ ID NO: 414; and a VL comprising therein a complementarity determining region (CDR) L1 having an amino acid sequence 100% identical to the amino acid sequence of SEQ ID NO: 416, a CDR L2 having an amino acid sequence 100% identical to the amino acid sequence of DAF, and a CDR L3 having an amino acid sequence 100% identical to the amino acid sequence of SEQ ID NO: 417. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 411 and comprising therein a complementarity determining region (CDR) H1 having an amino acid sequence 100% identical to the amino acid sequence of SEQ ID NO: 412, a CDR H2 having an amino acid sequence 100% identical to the amino acid sequence of SEQ ID NO: 413, and a CDR H3 having an amino acid sequence 100% identical to the amino acid sequence of SEQ ID NO: 414; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 415 and comprising therein a complementarity determining region (CDR) L1 having an amino acid sequence 100% identical to the amino acid sequence of SEQ ID NO: 416, a CDR L2 having an amino acid sequence 100% identical to the amino acid sequence of DAF, and a CDR L3 having an amino acid sequence 100% identical to the amino acid sequence of SEQ ID NO: 417. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 418 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 422. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 418 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 422. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 418 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 422. In some embodiments, the antibody comprises a variable heavy chain (VH) region having an amino acid sequence with at least 95% (e.g., at least 95%, 96%, 97%, 98%, 99%, or more) identity to SEQ ID NO: 418 and a variable light chain (VL) region having an amino acid sequence with at least 95% (e.g., at least 95%, 96%, 97%, 98%, 99%, or more) identity to SEQ ID NO: 422. In some embodiments, the antibody comprises a variable heavy chain (VH) region having an amino acid sequence 100% identical to the amino acid sequence of SEQ ID NO: 418 and a variable light chain (VL) region having an amino acid sequence 100% identical to the amino acid sequence of SEQ ID NO: 422. In some embodiments, the antibody comprises a VH comprising therein a complementarity determining region (CDR) H1 having an amino acid sequence 100% identical to the amino acid sequence of SEQ ID NO: 419, a CDR H2 having an amino acid sequence 100% identical to the amino acid sequence of SEQ ID NO: 420, and a CDR H3 having an amino acid sequence 100% identical to the amino acid sequence of SEQ ID NO: 421; and a VL comprising therein a complementarity determining region (CDR) L1 having an amino acid sequence 100% identical to the amino acid sequence of SEQ ID NO: 423, a CDR L2 having an amino acid sequence 100% identical to the amino acid sequence of DAS, and a CDR L3 having an amino acid sequence 100% identical to the amino acid sequence of SEQ ID NO: 424. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 418 and comprising therein a complementarity determining region (CDR) H1 having an amino acid sequence 100% identical to the amino acid sequence of SEQ ID NO: 419, a CDR H2 having an amino acid sequence 100% identical to the amino acid sequence of SEQ ID NO: 420, and a CDR H3 having an amino acid sequence 100% identical to the amino acid sequence of SEQ ID NO: 421; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 422 and comprising therein a complementarity determining region (CDR) L1 having an amino acid sequence 100% identical to the amino acid sequence of SEQ ID NO: 423, a CDR L2 having an amino acid sequence 100% identical to the amino acid sequence of DAS, and a CDR L3 having an amino acid sequence 100% identical to the amino acid sequence of SEQ ID NO: 424. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 425 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 429. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 425 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 429. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 425 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 429. In some embodiments, the antibody comprises a variable heavy chain (VH) region having an amino acid sequence with at least 95% (e.g., at least 95%, 96%, 97%, 98%, 99%, or more) identity to SEQ ID NO: 425 and a variable light chain (VL) region having an amino acid sequence with at least 95% (e.g., at least 95%, 96%, 97%, 98%, 99%, or more) identity to SEQ ID NO: 429. In some embodiments, the antibody comprises a variable heavy chain (VH) region having an amino acid sequence 100% identical to the amino acid sequence of SEQ ID NO: 425 and a variable light chain (VL) region having an amino acid sequence 100% identical to the amino acid sequence of SEQ ID NO: 429. In some embodiments, the antibody comprises a VH comprising therein a complementarity determining region (CDR) H1 having an amino acid sequence 100% identical to the amino acid sequence of SEQ ID NO: 426, a CDR H2 having an amino acid sequence 100% identical to the amino acid sequence of SEQ ID NO: 427, and a CDR H3 having an amino acid sequence 100% identical to the amino acid sequence of SEQ ID NO: 428; and a VL comprising therein a complementarity determining region (CDR) L1 having an amino acid sequence 100% identical to the amino acid sequence of SEQ ID NO: 430, a CDR L2 having an amino acid sequence 100% identical to the amino acid sequence of DAS, and a CDR L3 having an amino acid sequence 100% identical to the amino acid sequence of SEQ ID NO: 431. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 425 and comprising therein a complementarity determining region (CDR) H1 having an amino acid sequence 100% identical to the amino acid sequence of SEQ ID NO: 426, a CDR H2 having an amino acid sequence 100% identical to the amino acid sequence of SEQ ID NO: 427, and a CDR H3 having an amino acid sequence 100% identical to the amino acid sequence of SEQ ID NO: 428; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 429 and comprising therein a complementarity determining region (CDR) L1 having an amino acid sequence 100% identical to the amino acid sequence of SEQ ID NO: 430, a CDR L2 having an amino acid sequence 100% identical to the amino acid sequence of DAS, and a CDR L3 having an amino acid sequence 100% identical to the amino acid sequence of SEQ ID NO: 431. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 432 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 436. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 432 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 436. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 432 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 436. In some embodiments, the antibody comprises a variable heavy chain (VH) region having an amino acid sequence with at least 95% (e.g., at least 95%, 96%, 97%, 98%, 99%, or more) identity to SEQ ID NO: 432 and a variable light chain (VL) region having an amino acid sequence with at least 95% (e.g., at least 95%, 96%, 97%, 98%, 99%, or more) identity to SEQ ID NO: 436. In some embodiments, the antibody comprises a variable heavy chain (VH) region having an amino acid sequence 100% identical to the amino acid sequence of SEQ ID NO: 432 and a variable light chain (VL) region having an amino acid sequence 100% identical to the amino acid sequence of SEQ ID NO: 436. In some embodiments, the antibody comprises a VH comprising therein a complementarity determining region (CDR) H1 having an amino acid sequence 100% identical to the amino acid sequence of SEQ ID NO: 433, a CDR H2 having an amino acid sequence 100% identical to the amino acid sequence of SEQ ID NO: 434, and a CDR H3 having an amino acid sequence 100% identical to the amino acid sequence of SEQ ID NO: 435; and a VL comprising therein a complementarity determining region (CDR) L1 having an amino acid sequence 100% identical to the amino acid sequence of SEQ ID NO: 437, a CDR L2 having an amino acid sequence 100% identical to the amino acid sequence of GAS, and a CDR L3 having an amino acid sequence 100% identical to the amino acid sequence of SEQ ID NO: 438. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 432 and comprising therein a complementarity determining region (CDR) H1 having an amino acid sequence 100% identical to the amino acid sequence of SEQ ID NO: 433, a CDR H2 having an amino acid sequence 100% identical to the amino acid sequence of SEQ ID NO: 434, and a CDR H3 having an amino acid sequence 100% identical to the amino acid sequence of SEQ ID NO: 435; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 436 and comprising therein a complementarity determining region (CDR) L1 having an amino acid sequence 100% identical to the amino acid sequence of SEQ ID NO: 437, a CDR L2 having an amino acid sequence 100% identical to the amino acid sequence of GAS, and a CDR L3 having an amino acid sequence 100% identical to the amino acid sequence of SEQ ID NO: 438. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 439 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 443. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 439 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 443. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 439 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 443. In some embodiments, the antibody comprises a variable heavy chain (VH) region having an amino acid sequence with at least 95% (e.g., at least 95%, 96%, 97%, 98%, 99%, or more) identity to SEQ ID NO: 439 and a variable light chain (VL) region having an amino acid sequence with at least 95% (e.g., at least 95%, 96%, 97%, 98%, 99%, or more) identity to SEQ ID NO: 443. In some embodiments, the antibody comprises a variable heavy chain (VH) region having an amino acid sequence 100% identical to the amino acid sequence of SEQ ID NO: 439 and a variable light chain (VL) region having an amino acid sequence 100% identical to the amino acid sequence of SEQ ID NO: 443. In some embodiments, the antibody comprises a VH comprising therein a complementarity determining region (CDR) H1 having an amino acid sequence 100% identical to the amino acid sequence of SEQ ID NO: 440, a CDR H2 having an amino acid sequence 100% identical to the amino acid sequence of SEQ ID NO: 441, and a CDR H3 having an amino acid sequence 100% identical to the amino acid sequence of SEQ ID NO: 442; and a VL comprising therein a complementarity determining region (CDR) L1 having an amino acid sequence 100% identical to the amino acid sequence of SEQ ID NO: 444, a CDR L2 having an amino acid sequence 100% identical to the amino acid sequence of AAS, and a CDR L3 having an amino acid sequence 100% identical to the amino acid sequence of SEQ ID NO: 445. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 439 and comprising therein a complementarity determining region (CDR) H1 having an amino acid sequence 100% identical to the amino acid sequence of SEQ ID NO: 440, a CDR H2 having an amino acid sequence 100% identical to the amino acid sequence of SEQ ID NO: 441, and a CDR H3 having an amino acid sequence 100% identical to the amino acid sequence of SEQ ID NO: 442; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 443 and comprising therein a complementarity determining region (CDR) L1 having an amino acid sequence 100% identical to the amino acid sequence of SEQ ID NO: 444, a CDR L2 having an amino acid sequence 100% identical to the amino acid sequence of AAS, and a CDR L3 having an amino acid sequence 100% identical to the amino acid sequence of SEQ ID NO: 445. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: SEQ 446 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 450. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97% 98%, 99%, or more) identity to SEQ ID NO: 446 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 450. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 446 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 450. In some embodiments, the antibody comprises a variable heavy chain (VH) region having an amino acid sequence with at least 95% (e.g., at least 95%, 96%, 97%, 98%, 99%, or more) identity to SEQ ID NO: 446 and a variable light chain (VL) region having an amino acid sequence with at least 95% (e.g., at least 95%, 96%, 97%, 98%, 99%, or more) identity to SEQ ID NO: 450. In some embodiments, the antibody comprises a variable heavy chain (VH) region having an amino acid sequence 100% identical to the amino acid sequence of SEQ ID NO: 446 and a variable light chain (VL) region having an amino acid sequence 100% identical to the amino acid sequence of SEQ ID NO: 450. In some embodiments, the antibody comprises a VH comprising therein a complementarity determining region (CDR) H1 having an amino acid sequence 100% identical to the amino acid sequence of SEQ ID NO: 447, a CDR H2 having an amino acid sequence 100% identical to the amino acid sequence of SEQ ID NO: 448, and a CDR H3 having an amino acid sequence 100% identical to the amino acid sequence of SEQ ID NO: 449; and a VL comprising therein a complementarity determining region (CDR) L1 having an amino acid sequence 100% identical to the amino acid sequence of SEQ ID NO: 451, a CDR L2 having an amino acid sequence 100% identical to the amino acid sequence of DNF, and a CDR L3 having an amino acid sequence 100% identical to the amino acid sequence of SEQ ID NO: 452. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 446 and comprising therein a complementarity determining region (CDR) H1 having an amino acid sequence 100% identical to the amino acid sequence of SEQ ID NO: 447, a CDR H2 having an amino acid sequence 100% identical to the amino acid sequence of SEQ ID NO: 448, and a CDR H3 having an amino acid sequence 100% identical to the amino acid sequence of SEQ ID NO: 449; a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 450 and comprising therein a complementarity determining region (CDR) L1 having an amino acid sequence 100% identical to the amino acid sequence of SEQ ID NO: 451, a CDR L2 having an amino acid sequence 100% identical to the amino acid sequence of DNF, and a CDR L3 having an amino acid sequence 100% identical to the amino acid sequence of SEQ ID NO: 452. In some embodiments, the antibody binds to one or more amino acid residues of the VWF CK domain selected from D2726, R2730, K2735, E2744, D2746, K2757, D2763, and T2789. In some embodiments, the antibody binds to at least the following amino acid residues of the VWF CK domain: D2726, R2730, D2746, and K2757. In some embodiments, the antibody binds to at least the following amino acid residues of the VWF CK domain: R2730, K2735, E2744, and D2746. In some embodiments, the antibody binds to at least the following amino acid residues of the VWF CK domain: K2735, D2746, and K2757. In some embodiments, the antibody binds to at least the following amino acid residues of the VWF CK domain: R2730, D2746, and K2757. In some embodiments, the antibody binds to at least the following amino acid residues of the VWF CK domain: K2735, E2744, D2746, and D2763. In some embodiments, the antibody binds to at least the following amino acid residues of the VWF CK domain: R2730, K2735, E2744, D2746, K2757, and D2763. In some embodiments, the antibody binds to at least amino acid residue K2735 of the VWF CK domain. In some embodiments, the antibody binds to at least the following amino acid residues of the VWF CK domain: D2726, K2735, and K2757. In some embodiments, the antibody binds to at least the following amino acid residues of the VWF CK domain: K2735 and D2763. In some embodiments, the modified Fc region increases the blood plasma half-life of the antibody as compared to a blood plasma half-life of an antibody lacking modifications in the Fc region. In some embodiments, the modified Fc region increases the blood plasma half-life of an antibody disclosed herein by at least 1.5-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold greater as compared to a blood plasma half-life of an antibody lacking modifications in the Fc region. In some embodiments, the modified Fc region increases the blood plasma half-life of an antibody disclosed herein by at least 5%, 10%, 20%, 30%, 40%, 50%, 60%, 70%, 80%, 90%, 100%, 150%, 200%, 250%, 300%, 350%, 400%, 450%, 500%, 550%, 600%, 650%, 700%, 750%, 800%, 850%, 900%, 950%, 1,000%, 1,500%, 2,000%, 2,500%, 3,000%, 3,500%, 4,000%, 4,500%, 5,000%, 5,500%, 6,000%, 6,500%, 7,000%, 7,500%, 8,000%, 8,500%, 9,000%, 9,500%, 10,000%, or greater as compared to a blood plasma half-life of an antibody lacking modifications in the Fc region. In some embodiments, the modified Fc region reduces one or more effector functions of an antibody disclosed herein as compared to the effector functions of an antibody lacking modifications in the Fc region. In some embodiments, the one or more effector functions are selected from the group consisting of antibody-dependent cellular cytotoxicity (ADCC), complement dependent cytotoxicity (CDC), opsonization, phagocytosis, transcytosis, neutralization of infectivity, inflammation, mucosal immunity, and neonatal immunity. In some embodiments, the modified Fc region reduces one or more effector functions of an antibody disclosed herein by at least 5%, 10%, 20%, 30%, 40%, 50%, 60%, 70%, 80%, 90%, or 100% as compared to the one or more effector functions of the antibody lacking modifications in the Fc region. In some embodiments, the modified Fc region reduces one or more effector functions of an antibody disclosed herein by at least 1.5-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold as compared to the one or more effector functions of the antibody lacking modifications in the Fc region. In some embodiments, binding of an antibody disclosed herein to the VWF protein does not result in a change in function of the VWF protein. In some embodiments, binding of an antibody disclosed herein to VWF (e.g., to VWF CK domain) does not inhibit VWF protein binding to one or more binding partners. In some embodiments, the one or more binding partners are selected from the group consisting of Factor VIII (FVIII), collagen, glycoprotein Ib (gp1b), glycoprotein IIb/IIIa (gpIIb/IIIa), and ADAM Metallopeptidase With Thrombospondin Type 1 Motif 13 (ADAMTS13). In some embodiments, binding of an antibody disclosed herein does not inhibit binding of the VWF protein to platelet cells. In some embodiments, the accumulation of the VWF protein in blood plasma results in a blood plasma level of the VWF protein that is at least 1.3-fold, 1.4-fold, 1.5-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold greater as compared to a blood plasma level of the VWF protein in the absence of the antibody. In some embodiments, the accumulation of the VWF protein in blood plasma results in a blood plasma level of the VWF protein that is at least 5%, 10%, 20%, 30%, 40%, 50%, 60%, 70%, 80%, 90%, 100%, 150%, 200%, 250%, 300%, 350%, 400%, 450%, 500%, 550%, 600%, 650%, 700%, 750%, 800%, 850%, 900%, 950%, 1,000%, 1,500%, 2,000%, 2,500%, 3,000%, 3,500%, 4,000%, 4,500%, 5,000%, 5,500%, 6,000%, 6,500%, 7,000%, 7,500%, 8,000%, 8,500%, 9,000%, 9,500%, 10,000%, or greater as compared to a blood plasma level of the VWF protein in the absence of the antibody. In some embodiments, binding of an antibody disclosed herein to VWF in blood plasma increases a blood plasma half-life of the VWF protein as compared to a blood plasma half-life of the VWF protein in the absence of the antibody. In some embodiments, binding of an antibody disclosed herein to VWF in blood plasma increases the blood plasma half-life of the VWF protein by at least 1.5-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold as compared to the blood plasma half-life of the VWF protein in the absence of the antibody. In some embodiments, binding of an antibody disclosed herein to VWF in blood plasma increases the blood plasma half-life of the VWF protein by at least 5%, 10%, 20%, 30%, 40%, 50%, 60%, 70%, 80%, 90%, 100%, 150%, 200%, 250%, 300%, 350%, 400%, 450%, 500%, 550%, 600%, 650%, 700%, 750%, 800%, 850%, 900%, 950%, 1,000%, 1,500%, 2,000%, 2,500%, 3,000%, 3,500%, 4,000%, 4,500%, 5,000%, 5,500%, 6,000%, 6,500%, 7,000%, 7,500%, 8,000%, 8,500%, 9,000%, 9,500%, or 10,000% as compared to the blood plasma half-life of the VWF protein in the absence of the antibody. In some embodiments, binding of an antibody disclosed herein to the VWF protein in blood plasma results in accumulation of a FVIII protein in blood plasma as compared to a blood plasma level of a FVIII protein in the absence of the antibody. In some embodiments, the accumulation of the FVIII protein in blood plasma results in a blood plasma level of the FVIII protein that is at least 1.3-fold, 1.4-fold, 1.5-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold greater as compared to a blood plasma level of the FVIII protein in the absence of the antibody. In some embodiments, the accumulation of the FVIII protein in blood plasma results in a blood plasma level of the FVIII protein that is at least 5%, 10%, 20%, 30%, 40%, 50%, 60%, 70%, 80%, 90%, 100%, 150%, 200%, 250%, 300%, 350%, 400%, 450%, 500%, 550%, 600%, 650%, 700%, 750%, 800%, 850%, 900%, 950%, 1,000%, 1,500%, 2,000%, 2,500%, 3,000%, 3,500%, 4,000%, 4,500%, 5,000%, 5,500%, 6,000%, 6,500%, 7,000%, 7,500%, 8,000%, 8,500%, 9,000%, 9,500%, 10,000%, or greater as compared to the blood plasma level of a FVIII protein in the absence of the antibody. In some embodiments, binding of an antibody disclosed herein to VWF in blood plasma increases a blood plasma half-life of FVIII protein as compared to a blood plasma half-life of the FVIII protein in the absence of the antibody. In some embodiments, binding of an antibody disclosed herein to VWF in blood plasma increases the blood plasma half-life of the FVIII protein by at least 1.5-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold as compared to the blood plasma half-life of the FVIII protein in the absence of the antibody. In some embodiments, binding of an antibody disclosed herein to VWF in blood plasma increases the blood plasma half-life of the FVIII protein by at least 5%, 10%, 20%, 30%, 40%, 50%, 60%, 70%, 80%, 90%, 100%, 150%, 200%, 250%, 300%, 350%, 400%, 450%, 500%, 550%, 600%, 650%, 700%, 750%, 800%, 850%, 900%, 950%, 1,000%, 1,500%, 2,000%, 2,500%, 3,000%, 3,500%, 4,000%, 4,500%, 5,000%, 5,500%, 6,000%, 6,500%, 7,000%, 7,500%, 8,000%, 8,500%, 9,000%, 9,500%, or 10,000% as compared to the blood plasma half-life of the FVIII protein in the absence of the antibody. In some embodiments, an antibody disclosed herein binds specifically to a VWF homodimer comprising two VWF monomers linked tail-to-tail via CK-domain joining in a 1:1 stoichiometry. In some embodiments, an antibody disclosed herein binds specifically to a VWF homodimer comprising two VWF monomers linked tail-to-tail via CK domain joining in 2:1 stoichiometry. In some embodiments, an antibody disclosed herein binds to the CK domain of the VWF protein with a dissociation constant (K_{d}) of less than 100 nM, 90 nM, 80 nM, 70 nM, 60 nM, 50 nM, 40 nM, 30 nM, 20 nM, 10 nM, 7 nM, 5 nM, 4 nM, 3 nM, 2 nM, 1 nM, 0.5 nM, 0.25 nM, 0.1 nM, 0.075 nM, 0.05 nM, 0.025 nM, 0.01 nM, or less. In some embodiments, an antibody disclosed herein is cross-reactive with a VWF protein of Macaca fascicularis, Mus musculus, and/or Canis familiaris. In some embodiments, an antibody disclosed herein has an affinity with a VWF protein of Macaca fascicularis, Mus musculus, and/or Canis familiaris that is at most 10-fold (e.g., at most 10-fold, 9-fold, 8-fold, 7-fold, 6-fold, 5-fold, 4-fold, 3-fold, 2-fold, 1.5-fold, or less) lower than an affinity of the antibody to a human VWF protein. In some embodiments, an antibody disclosed herein is a human antibody, humanized antibody, or chimeric antibody. In some embodiments, the CK domain is positioned between amino acid positions 2,723 and 2,813 of SEQ ID NO: 1. In some embodiments, the CK domain is a CK domain of a human VWF protein having at least 85% sequence identity (e.g., at least 85%, 86%, 87%, 88%, 89%, 90%, 91%, 92%, 93%, 94%, 95%, 96%, 97%, 98%, 99%, or more) to the amino acid sequence of any one of SEQ ID NOs: 5, 6, 486, or 487. In some embodiments, the CK domain has an amino acid sequence of SEQ ID NO: 5. In some embodiments, the CK domain is a CK domain of a non-human primate (NHP) VWF protein having at least 85% sequence identity (e.g., at least 85%, 86%, 87%, 88%, 89%, 90%, 91%, 92%, 93%, 94%, 95%, 96%, 97%, 98%, 99%, or more) to the amino acid sequence of SEQ ID NO: 6. In some embodiments, the CK domain has an amino acid sequence of SEQ ID NO: 6.

Disclosed herein, in certain embodiments, is a complex comprising a VWF protein and a monospecific antibody of the foregoing aspects and embodiments. In some embodiments, the complex comprises a VWF monomer. In some embodiments, the complex comprises a VWF dimer. In some embodiments, the complex comprises a VWF multimer. In some embodiments, the multimer has a molecular weight of at least 250 kDa (e.g., at least 250 kDa, 500 kDa, 750 kDa, 1000 kDa, 1,250 kDa, 1,500 kDa, 1,750 kDa, 2,000 kDa, 3,000 kDa, 4,000 kDa, 5,000 kDa, 6,000 kDa, 7,000 kDa, 8,000 kDa, 9,000 kDa, 10,000 kDa, 11,000 kDa, 12,000 kDa, 13,000 kDa, 14,000 kDa, 15,000 kDa, 16,000 kDa, 17,000 kDa, 18,000 kDa, 19,000 kDa, 20,000 kDa, 21,000 kDa, 22,000 kDa, 23,000 kDa, 24,000 kDa, 25,000 kDa, or more).

Disclosed herein, in certain embodiments, are pharmaceutical compositions comprising a monospecific antibody of the foregoing aspects and embodiments and a pharmaceutically acceptable carrier, diluent, or excipient. In some embodiments, the pharmaceutically acceptable carrier, diluent, or excipient is selected from the group consisting of a stabilizer, buffer, surfactant, filler, solvent, tonicity or osmolarity adjusting agent, antioxidant, adjuvant, and antimicrobial agent.

Disclosed herein, in certain embodiments, are methods of treating a blood disorder characterized by excessive bleeding in a subject in need thereof, comprising administering to the subject a monospecific antibody or a pharmaceutical composition of the foregoing aspects and embodiments. In some embodiments, the blood disorder is selected from the group consisting of von Willebrand disease (VWD), Heydes syndrome, hemophilia A, disorders of platelet function, and connective tissue disorders. In some embodiments, the VWD is congenital VWD (cVWD) or acquired VWD (aVWD). In some embodiments, the VWD is type 1, type 2A, type 2B, type 2M, type 2N, or type 3. In some embodiments, a composition disclosed herein treats one or more (e.g., 1, 2, 3, 4, 5, 6, 7, 8, 9, 10, or more) symptoms of the blood disorder selected from the group consisting of epistaxis, cutaneous bleeding, bleeding from minor wounds, oral-cavity bleeding, angiodysplasia, gastrointestinal bleeding, bleeding from tooth extraction, postoperative bleeding, heavy menstrual bleeding, obstetric hemorrhage, hematuria, muscle hematoma, joint bleeding, visceral bleeding, and central nervous system (CNS) bleeding. In some embodiments, a composition or pharmaceutical composition disclosed herein promotes platelet adhesion, activation, and aggregation in the subject.

Disclosed herein, in certain embodiments, are methods of accumulating a VWF protein in blood plasma of a subject in need thereof, comprising administering to the subject a composition comprising a monospecific antibody of the foregoing aspects and embodiments.

Disclosed herein, in certain embodiments, are methods of accumulating a FVIII protein in blood plasma of a subject in need thereof, comprising administering to the subject a composition comprising a monospecific antibody of the foregoing aspects and embodiments.

Disclosed herein, in certain embodiments, are methods of increasing blood plasma half-life of a VWF protein in a subject in need thereof, comprising administering to the subject a composition comprising a monospecific antibody of the foregoing aspects and embodiments.

Disclosed herein, in certain embodiments, are pharmaceutical compositions comprising a monospecific antibody disclosed herein and a pharmaceutically acceptable carrier, diluent, or excipient. In some embodiments, the pharmaceutically acceptable carrier, diluent, or excipient is selected from the group consisting of a stabilizer, buffer, surfactant, filler, solvent, tonicity or osmolarity adjusting agent, antioxidant, adjuvant, and antimicrobial agent. In some embodiments, a pharmaceutical composition disclosed herein is administered in combination with one or more (e.g., 1, 2, 3, 4, 5, or more) additional therapeutic modalities. In some embodiments, the one or more additional therapeutic modalities is one or more therapeutic agents. In some embodiments, the one or more therapeutic agents is selected from the group consisting of desmopressin (DDAVP), tranexamic acid (TXA), thalidomide, tamoxifen, ocreotide, a concentrate of plasma-derived VWF protein, a concentrate of VWF protein in complex with FVIII (VWF:FVIII; e.g., Humate P and Wilate), a concentrate of recombinant VWF protein (e.g., Vonicog alfa), a concentrate of FVIII protein (e.g., Alphanate and Koate-HP), hormonal treatment (e.g., an oral contraceptive pill, e.g., an estrogen-containing contraceptive and an intrauterine device). In some embodiments, the one or more additional therapeutic modalities is surgery. In some embodiments, the one or more additional therapeutic modalities is blood transfusion. In some embodiments, the one or more additional therapeutic modalities is platelet transfusion. In some embodiments, the one or more additional therapeutic modalities is cauterization. In some embodiments, the one or more additional therapeutic modalities is gene therapy. In some embodiments, the gene therapy is FVIII gene therapy. For example, an antibody or composition of the disclosure may be administered in combination with a FVIII gene therapy to extend the half-life of FVIII and/or promote accumulation of FVIII in blood plasma of a subject. In some embodiments, an antibody or composition of the disclosure is administered to a patient for whom FVIII gene therapy alone is insufficient to attain therapeutically effective levels of FVIII in blood plasma. In some embodiments, an antibody or composition of the disclosure achieves therapeutically effective levels of FVIII in blood plasma. In some embodiments, the one or more additional therapeutic modalities are administered to the subject prior to, concurrently with, or subsequent to administration of the composition or the pharmaceutical composition. In some embodiments, the one or more additional therapeutic modalities are administered to the subject prior to administration of the composition or the pharmaceutical composition. In some embodiments, the one or more additional therapeutic modalities are administered to the subject concurrently with the composition or the pharmaceutical composition. In some embodiments, the one or more additional therapeutic modalities are administered to the subject subsequent to administration of the composition or the pharmaceutical composition.

Disclosed herein, in certain embodiments, are kits comprising a monospecific antibody or a pharmaceutical composition of the foregoing aspects and embodiments, and a package insert instructing a user of the kit to perform a method of the foregoing aspects and embodiments.

Disclosed herein, in certain embodiments, are monospecific antibodies and pharmaceutical compositions of the foregoing embodiments for use as a medicament. In some embodiments, a monospecific antibody or the pharmaceutical composition of the foregoing embodiments is for use in the treatment of a blood disorder characterized by excessive bleeding.

Disclosed herein, in certain embodiments, are monospecific antibodies or pharmaceutical compositions of the foregoing embodiments for use in the treatment of a blood disorder characterized by excessive bleeding.

In some embodiments of the foregoing aspects, the blood disorder characterized by excessive bleeding is selected from the group consisting of VWD, Heydes syndrome, hemophilia A, disorders of platelet function, and connective tissue disorders. In some embodiments, the VWD is cVWD or aVWD. In some embodiments, the VWD is type 1, type 2A, type 2B, type 2M, type 2N, or type 3.

### Definitions

Unless defined otherwise, all technical and scientific terms used herein have the same meaning as is commonly understood by one of skill in the art to which the claimed subject matter belongs. Generally, nomenclatures utilized in connection with, and techniques of, immunology, oncology, cell and tissue culture, molecular biology, and protein and oligo- or polynucleotide chemistry and hybridization described herein are those well-known and commonly used in the art. It is to be understood that the foregoing general description and the following detailed description are exemplary and explanatory only and are not restrictive of any subject matter claimed. The section headings used herein are for organizational purposes only and are not to be construed as limiting the subject matter described.

As used herein, singular forms “a,” “and,” and “the” include plural referents unless the context clearly indicates otherwise. Thus, e.g., reference to “an antibody” includes a plurality of antibodies and reference to “an antibody” in some embodiments includes multiple antibodies, and so forth.

As used herein, all numerical values or numerical ranges include whole integers within or encompassing such ranges and fractions of the values or the integers within or encompassing ranges unless the context clearly indicates otherwise. Thus, e.g., reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. In another example, reference to a range of 1-5,000 fold includes 1, 2, 3, 4, 5, 6, 7, 8, 9, 10, 11, 12, 13, 14, 15, 16, 17, 18, 19, 20 fold, etc., as well as 1.1, 1.2, 1.3, 1.4, 1.5 fold, etc., 2.1, 2.2, 2.3, 2.4, 2.5 fold, etc., and so forth. [00019]“About” a number, as used herein, refers to range including the number and ranging from 10% below that number to 10% above that number. “About” a range refers to 10% below the lower limit of the range, spanning to 10% above the upper limit of the range.

As used herein, the term “accumulation” refers to an increase in a level (e.g., concentration) of a particular protein, such as, e.g., VWF or FVIII, in a blood compartment (e.g., blood plasma) of a subject following administration of an antibody of the disclosure or a composition (e.g., pharmaceutical composition) containing the same to the subject. Various factors may influence the rate of accumulation of a protein in a blood compartment of a subject, including but not limited to, the half-life of the target protein and/or the antibody in the blood compartment, the rate of sequestration of the protein by other tissues, clearance of the protein from the body, among others. According to the present disclosure, accumulation of a target protein (e.g., VWF or FVIII) may result in a blood plasma level of the protein that is at least 1.5-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold greater as compared to a blood plasma level of the protein in the absence of the antibody. Alternatively, accumulation of the target protein may results in a blood plasma level of the protein that is at least 5%, 10%, 20%, 30%, 40%, 50%, 60%, 70%, 80%, 90%, 100%, 150%, 200%, 250%, 300%, 350%, 400%, 450%, 500%, 550%, 600%, 650%, 700%, 750%, 800%, 850%, 900%, 950%, 1,000%, 1,500%, 2,000%, 2,500%, 3,000%, 3,500%, 4,000%, 4,500%, 5,000%, 5,500%, 6,000%, 6,500%, 7,000%, 7,500%, 8,000%, 8,500%, 9,000%, 9,500%, 10,000%, or greater as compared to a blood plasma level of the protein in the absence of the antibody. In some embodiments, accumulation of a protein (e.g., VWF) results directly from specific binding of an antibody of the disclosure to the protein. In some embodiments, accumulation of a protein (e.g., FVIII) results indirectly from binding of the protein (e.g., FVIII) to another protein (e.g., VWF) bound by an antibody of the disclosure.

As used herein “antibody” refers to a naturally-occurring or non-naturally-occurring protein that binds an antigen. An antibody often comprises a variable domain and a constant domain in each of a heavy chain and a light chain. Accordingly, most antibodies have a heavy chain variable domain (V_{H}) and a light chain variable domain (V_{L}) that together form the portion of the antibody that binds to the antigen, sometimes referred to as the “antigen receptor.” Within each variable domain are three complementarity-determining regions (CDR), which form loops in the V_{H} and V_{L} and contact the surface of the antigen. “Antibody” includes, but is not limited to monoclonal antibodies, monospecific antibodies, monovalent antibodies, multispecific antibodies (e.g., bispecific antibodies), humanized antibodies, chimeric antibodies, synthetic antibodies, recombinant antibodies, hybrid antibodies, mutated antibodies, grafted antibodies, antibody fragments, and in vitro-generated antibodies having the antigen-binding activity. The term also includes antibody conjugates having advantageous properties as compared to an unconjugated antibody (e.g., antibodies conjugated to a half-life extending moiety, e.g., a fatty acid)).

As used herein, “complementarity-determining regions,” “CDRs,” and “hypervariable regions” refer to the parts of the variable domains in antibodies that determine the antibodies' binding specificities to their specific antigen. A single variable region of an antibody polypeptide will typically comprise three CDRs, usually designated CDR1, CDR2, and CDR3. More particularly, a heavy chain variable region may contain CDRs designated H1, H2, and H3; likewise, light chain variable region may contain CDRs L1, L2, and L3. Multiple methods may be used to define a CDR. The current art utilizes various numbering schemes with different definitions of CDR lengths and positions. For example, IMGT numbering scheme is a standardized numbering system based on alignments of sequences from a complete reference gene database including the whole immunoglobulin superfamily. The Kabat numbering scheme is based on sequence alignment and uses “variability parameter” of a given amino acid position (the number of different amino acids at a given position divided by the frequency of the most occurring amino acid at that position) to predict CDRs. The Chothia numbering scheme, on the other hand, is a structure-based numbering scheme where antibody crystal structures are aligned as define the loop structures as CDRs. The Martin numbering scheme focuses on the structure alignment of different framework regions of unconventional lengths. Honneger's numbering scheme (AHo's) is based on structural alignments of the 3D structure of the variable regions and uses structurally conserved Ca positions to deduce framework and CDR lengths. One of skill in the art will note that the definition of a CDR will vary based on the method used. Accordingly, CDR sequences of a given heavy or light chain variable region may vary depending on the numbering system used. Any method of defining a CDR is contemplated with the sequences disclosed herein. Unless stated otherwise, the CDRs disclosed herein are defined according to the IMGT numbering scheme.

The terms “antigen-binding portion of an antibody,” “antigen-binding fragment,” “antigen-binding domain,” “antibody fragment” are used interchangeably herein to refer to one or more fragments of an antibody that retain the ability to specifically bind to the antigen. Non-limiting examples of antibody fragments included within such terms include, but are not limited to, e.g., a Fab fragment, a monovalent fragment consisting of the V_{L}, V_{H}, C_{L}, and C_{H1} domains. Also included are “one-arm” antibodies comprising a single heavy chain, a truncated heavy chain lacking a Fab region, and a single light chain.

As used herein, the phrase “blood disorder characterized by excessive bleeding” refers to one or more disorders in a subject (e.g., a human) that confers a susceptibility to bleed to the subject (e.g., bleeding diathesis). Such a disorder is, in some embodiments, due to a condition associated with atypical and slow blood clotting. A “blood disorder characterized by excessive bleeding” may be genetic or acquired in origin. Platelet activation, adhesion, and/or aggregation (e.g., primary hemostasis) is, in some embodiments, impaired in a subject with said disorder. In some embodiments, function or expression of blood proteins that mediate or facilitate the production of insoluble cross-linked fibrin proteins during blood coagulation (e.g., secondary hemostasis) is impaired in a subject with the disorder. Non-limiting examples of a blood disorder characterized by excessive bleeding include von Willebrand disease (VWD), hemophilia A, disorders of platelet function, and connective tissue disorders. In some embodiments, the VWD is congenital VWD (cVWD) or acquired VWD (aVWD). In some embodiments, the aVWD includes Heydes syndrome. In some embodiments, the VWD is type 1, type 2A, type 2B, type 2M, type 2N, or type 3.

As used herein, the term “change in function” when referring to the VWF protein means an increase or a decrease by any amount (e.g., increase or decrease by 5%, 10%, 15%, 20%, 25%, 30%, 35%, 40%, 45%, 50%, 55%, 60%, 65%, 70%, 75%, 80%, 85%, 90%, 95%, 100%, or increase by 150%, 200%, 250%, 300%, 350%, 400%, 450%, 500%, 600%, 700%, 800%, 900%, 1000%, 2000%, 3000%, 4000%, 5000%, 6000%, 7000%, 8000%, 9000%, 10000%, or more) in the intrinsic function of the VWF protein when bound to an antibody of the disclosure. VWF lacks enzymatic activity and its function is associated with binding and stabilization of other proteins involved in blood clotting. Therefore, the function of VWF is primarily to bind to other proteins, including binding partners described herein. Accordingly, a “change in function” of VWF corresponds to an increase or decrease in the ability of VWF to bind to other proteins, such as, e.g., its binding partners.

As used herein, the term “complex” refers to a molecular entity formed by two or more physically associated molecules (e.g., proteins), such as, e.g., a VWF protein and an antibody described herein. The two or more molecules, in some embodiments, are associated by any physical means, including covalent bonds, ionic bonds, hydrogen bonds, and van der Waal forces. In some embodiments, a physical association between the two or more molecules is reversible. In some embodiments, a physical association between the two or more molecules is irreversible.

The term “effective amount” as used herein, refers to that amount of an antibody, or an antigen-binding portion thereof as described herein, that is sufficient to induce a disclosed effect, e.g., to effect treatment (e.g., accumulation of a VWF and/or FVIII protein in blood plasma), prognosis, or diagnosis of a disease (e.g., a blood disorder characterized by excessive bleeding), as described herein, when administered to a subject. Therapeutically effective amounts of antibodies provided herein, when used alone or in combination, will vary depending upon the relative activity of the disclosed antibodies and combinations (e.g., in treating, reducing, or ameliorating a disease or disorder described herein) and depending upon the subject and disease condition being treated, the weight and age of the subject, the severity of the disease condition, the manner of administration, and the like, which, in some cases, are readily determined by one of ordinary skill in the art.

As used herein, the term “monospecific” refers to an antibody that displays a preferential affinity for one particular epitope (e.g., an epitope contained in a CK domain of the VWF protein, such as, e.g., a human, NHP, murine, or canine VWF protein).

As used herein, the term “monovalent” refers to a property of an antibody or antigen-binding fragment thereof in which the antibody has one antigen-binding site to a target epitope of a particular protein. For example, an monovalent antibody of the disclosure binds to an epitope (e.g., an epitope in a CK domain) of a VWF protein with a single antigen-binding site. Within the context of the present disclosure, a monovalent antibody is, in some embodiments, a one-armed antibody (e.g., having a heavy chain, a truncated heavy chain lacking a Fab region, and a single light chain).

As used herein, the terms “one-armed antibody” or “single-armed antibody” refer to a type of monovalent antibody fragment containing an antibody heavy chain, a truncated heavy chain lacking a Fab region, and a single light chain.

“Percent identity” and “% identity” refers to the extent to which two sequences (nucleotide or amino acid) have the same residue at the same positions in an alignment. For example, “an amino acid sequence is X % identical to SEQ ID NO: Y” refers to % identity of the amino acid sequence to SEQ ID NO: Y and is elaborated as X % of residues in the amino acid sequence are identical to the residues of sequence disclosed in SEQ ID NO: Y. Generally, computer programs are employed for such calculations. Exemplary programs that compare and align pairs of sequences, include ALIGN (Myers and Miller, Comput Appl Biosci. 1988 March; 4(1):11-7), FASTA (Pearson and Lipman, Proc Natl Acad Sci USA. 1988 April; 85(8):2444-8; Pearson, Methods Enzymol. 1990; 183:63-98), gapped BLAST (Altschul et al., Nucleic Acids Res. 1997 Sep. 1; 25(17): 3389-40), BLASTP, BLASTN, or GCG (Devereux et al., Nucleic Acids Res. 1984 Jan. 11; 12(1 Pt 1):387-95).

The term “polypeptide” refers to a chain of amino acids (e.g., naturally-occurring or non-naturally-occurring amino acids). In some embodiments, the polypeptide comprises amino acids that are naturally-occurring. In some embodiments, the polypeptide comprises amino acids that are non-naturally-occurring (cystine, desmosine, isodesmosine, hydroxyproline, hydroxylysine, gamma-carboxyglutamate, phosphoserine, phosphothreonine, phosphotyrosine, N-acetyl lysine, methyllysine, inositol, L-homoalanine, L-2-aminobutyric acid, L-azidohomoalanine, beta-phenylalanine, norleucine, 3-fluorotyrosine, 4-fluorophenylalanine, L-3,4-dyhydroxyphenylalanine, pNCSF, L-(7-hydroxycoumarin-4-yl)ethylglycine, 2,3-dihydroxypropyl cysteine, arylglycine, PrDiAzK, (S)—N-(2-Benzoylphenyl)-1-(2-flurobenzyl)-pyrolidine-2-carboxamide, 4-Phenanthracen-9-yl-1-phenylalanine, BN tryptophan analog, p-acetylphenylalanine, L-azidohomoalanine, among others). The polypeptides are not limited to a specific length of the product (e.g., polypeptides can comprise at least 2 amino acids linked together by way of peptide bonds). Peptides, oligopeptides, and proteins are included within the definition of polypeptide, and such terms are used interchangeably herein unless specifically indicated otherwise. This term also encompasses chains of peptides with post-expression modifications, e.g., glycosylation, acetylation, phosphorylation, and the like, as well as other modifications known in the art, both naturally occurring and non-naturally occurring. In some embodiments, a polypeptide is an entire protein, or a fragment thereof.

The terms “preferentially binds” or “specifically binds” mean that the antibodies or fragments thereof bind to an epitope with greater affinity than it binds unrelated amino acid sequences, and, if cross-reactive to other polypeptides containing the epitope, are not toxic at the levels at which they are formulated for administration to human use. In some embodiments, such affinity is at least 1.5-fold greater, 2-fold greater, at least 3-fold greater, at least 4-fold greater, at least 5-fold greater, at least 6-fold greater, at least 7-fold greater, at least 8-fold greater, at least 9-fold greater, 10-fold greater, at least 20-fold greater, at least 30-fold greater, at least 40-fold greater, at least 50-fold greater, at least 58-fold greater, at least 70-fold greater, at least 80-fold greater, at least 90-fold greater, at least 94-fold greater, or at least 1000-fold greater than the affinity of the antibody or fragment thereof for unrelated amino acid sequences.

The terms “recipient,” “individual,” “subject,” “host,” and “patient,” are used interchangeably herein and refer to any mammalian subject for whom diagnosis, treatment, or therapy is desired, particularly humans. “Mammal” for purposes of treatment refers to any animal classified as a mammal, including humans, domestic and farm animals, and laboratory, zoo, sports, or pet animals, such as dogs, horses, cats, cows, sheep, goats, pigs, mice, rats, rabbits, guinea pigs, monkeys, etc. In some embodiments, the mammal is a human. None of the terms require the supervision of a medical professional.

The term “specific” refers to a situation in which an antibody will preferentially bind to molecules other than the antigen containing the epitope recognized by the antibody. The term is also applicable where e.g., an antigen-binding domain is specific for a particular epitope which is carried by a number of antigens, in which case the antibody or antigen-binding fragment thereof carrying the antigen-binding domain will be able to bind to the various antigens carrying the epitope.

The term “therapeutically effective amount” generally refers to an amount of a disclosed antibody or a drug effective to “treat” a disease or disorder in a subject or mammal. In some embodiments, a composition described herein is administered to a subject in an amount that is effective for producing some desired therapeutic effect by inhibiting a disease or disorder as described herein at a reasonable benefit/risk ratio applicable to any medical treatment. A therapeutically effective amount is an amount that achieves at least partially a desired therapeutic or prophylactic effect in an organ or tissue. The amount of an antibody necessary to bring about prevention and/or therapeutic treatment of a disease or disorder is not fixed per se. In some embodiments, the amount of the antibody administered varies with the type of disease, extensiveness of the disease, and size of the mammal suffering from the disease or disorder. When used in conjunction with therapeutic methods involving administration of a therapeutic agent after the subject presents symptoms of a disease or disorder, the term “therapeutically effective” means that, after treatment, one or more signs or symptoms of the disease or disorder is ameliorated or eliminated.

An effective response of the present disclosure is achieved when the subject experiences partial or total alleviation or reduction of signs or symptoms of illness and, in the case of the treatment of a disease (e.g., a blood disorder characterized by excessive bleeding), specifically includes, without limitation, amelioration of symptoms, prolongation of progression, cure, remission, prolongation of survival, or other objective responses. In some embodiments, the expected progression-free survival times are measured in months to years, depending on prognostic factors including the number of relapses, stage of disease, and other factors. Prolonging survival includes without limitation times of at least 1 month (mo.), about at least 2 mos., about at least 3 mos., about at least 4 mos., about at least 6 mos., about at least 1 year, about at least 2 years, about at least 3 years, etc. Overall survival is also measured, e.g., in months to years. Alternatively, an effective response, in some embodiments, is that a subject's symptoms remain static. Further indications of treatment of indications are described in more detail below.

In some embodiments, administration of a therapeutic agent in a prophylactic method occurs prior to the manifestation of symptoms of an undesired disease or disorder, such that the disease or disorder is prevented or, alternatively, delayed in its progression. Thus, when used in conjunction with prophylactic methods, the term “therapeutically effective” means that, after treatment, a smaller number of subjects (on average) develop the undesired disease or disorder or progress in severity of symptoms.

As used herein, the terms “treatment,” “treating,” and the like, in some cases, refer to administering an agent, or carrying out a procedure, for the purposes of obtaining an effect. The effect may be prophylactic in terms of completely or partially preventing a disease or symptom thereof and/or is therapeutic in terms of effecting a partial or complete cure for a disease and/or symptoms of the disease. “Treatment,” as used herein, includes treatment of a disease or disorder (e.g., blood disorder characterized by excessive bleeding) in a mammal, particularly in a human, and includes: (a) preventing the disease or a symptom of a disease from occurring in a subject which is predisposed to the disease but has not yet been diagnosed as having it (e.g., including diseases that is associated with or caused by a primary disease; (b) inhibiting the disease, i.e., arresting its development; and (c) relieving the disease, i.e., causing regression of the disease. The term treating includes to any indicia of success in the treatment or amelioration or prevention of a disease or disorder, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms is based on one or more objective or subjective parameters, including the results of an examination by a physician. Accordingly, the term “treating” includes the administration of the agents of the present disclosure to prevent or delay, to alleviate, or to arrest or inhibit development of the symptoms or conditions associated with diseases. The term “therapeutic effect” refers to the reduction, elimination, or prevention of the disease, symptoms of the disease, or side effects of the disease in the subject. A subject is “treated” for a disease or disorder if, after receiving a therapeutic amount of an antibody of the present disclosure, the patient shows observable and/or measurable change in a parameter or symptom of the disease or disorder.

### BRIEF DESCRIPTION OF THE DRAWINGS

FIG. 1 shows a flowchart of the protocol used for selection of antibodies that specifically bind to the cysteine knot (CK) domain of human von Willebrand Factor (VWF) protein.

FIG. 2 shows an overview of the experimental setup used for measurement of the apparent equilibrium dissociation constant (K_{d}) between monovalent (one-arm; OA) anti-VWF CK domain antibody and CK domain of VWF.

FIG. 3 shows a bar graph illustrating the distribution of low molecular weight (LMW), intermediate molecular weight (IMW), and high molecular weight (HMW) VWF multimers following overnight incubation of normal human plasma with 600 nM of monovalent (one-arm) antibody and 1.5 mg/mL ristocetin. Two control conditions include plasma without antibody and ristocetin (Untreated) and plasma treated with ristocetin only (Control). Results are shown as mean±standard deviation across plasma from three healthy human donors.

FIGS. 4A-4E are plots showing pharmacokinetics and pharmacodynamics of monovalent (one-arm) Lib1_P1_A6 (Lib_P1_A6 OA) antibody administered subcutaneously at 4 mg/kg on day 1 and 2 to two cynomolgus monkeys (“cynos”; 3FG2 and 4FG2, respectively). Plasma levels of (FIG. 4A) Lib1_P1_A6 OA (Example 20), (FIG. 4B) VWF antigen (VWF:Ag; Example 15), (FIG. 4C) VWF ristocetin cofactor activity (VWF:RCo; Example 18), (FIG. 4D) distribution of low (LMW), intermediate (IMW) and high (HMW) molecular weight VWF multimers (Example 19), and (FIG. 4E) FVIII antigen (FVIII:Ag; Example 20).

FIGS. 5A-5E are plots showing pharmacokinetics and pharmacodynamics of monovalent (one-arm) Lib1_P1_A9 (Lib_P1_A9 OA) antibody administered subcutaneously at 4 mg/kg on day 1 and 2 to two cynos (1MG1 and 2FG1, respectively). Plasma levels of (FIG. 5A) Lib1_P1_A9 OA (Example 21), (FIG. 5B) VWF antigen (VWF:Ag; Example 15), (FIG. 5C) VWF ristocetin cofactor activity (VWF:RCo; Example 18), (FIG. 5D) distribution of low (LMW), intermediate (IMW) and high (HMW) molecular weight VWF multimers (Example 17), and (FIG. 5E) FVIII antigen (FVIII:Ag; Example 18).

### DETAILED DESCRIPTION

Disclosed herein, in certain embodiments, are antibodies that specifically bind to von Willebrand Factor (VWF). Also disclosed herein are pharmaceutical compositions containing the disclosed antibody and one or more pharmaceutically acceptable carriers, diluents or excipients. The disclosure also provides methods of using the disclosed antibodies or compositions containing the same for the treatment of a blood disorder characterized by excessive bleeding (e.g., von Willebrand disease (VWD), Heydes syndrome, hemophilia A, disorders of platelet function, and connective tissue disorders). A subject with a blood disorder characterized by excessive bleeding is treated in accord with the methods disclosed herein by administering an antibody of the disclosure or a composition containing the same (e.g., a pharmaceutical composition) to the subject by any acceptable route (e.g., subcutaneously).

### Regulation of Hemostasis

Blood circulation provides cells and tissues with necessary substances such as nutrients and oxygen and disposes of metabolic waste products from these cells. The blood vasculature establishes a physical barrier that ensures the containment of blood, regulation of its pressure throughout the organism, and tissue-directed delivery of nutrients and waste products. Disruption or damage to this barrier can result in potentially life-threatening blood loss. Blood plasma contains numerous soluble proteins that act in a concerted cascade of enzymatic reactions to produce a blood clot that can readily “plug” the damaged blood vessel and prevent or reduce further blood loss. This plug is stabilized by the proteolytic cleavage of fibrinogen into fibrin, which forms a mesh that ensheathes and reinforces the clot. Formation and degradation of blood clots are part of a process called hemostasis, which is regulated by a number of soluble and membrane-tethered pro-coagulant and anti-coagulant proteins. A balance between coagulation and fibrinolysis (i.e., degradation of insoluble fibrin proteins) controls the stability of the blood clot.

There are two central pathways that trigger blood clotting, namely the tissue factor (or extrinsic) pathway and the contact (or intrinsic) pathway. These clotting cascades proceed by sequential activation of zymogens (i.e., inactive protein precursors) by proteolytic cleavage. Each zymogen, when activated, acts as a serine protease that activates downstream zymogens to drive clot formation. Protein cofactors also act in the blood clotting cascade, generally circulating as inactive pro-cofactors in blood plasma until activated by limited proteolysis.

The extrinsic pathway is initiated when a cell-surface complex of an integral membrane protein, tissue factor (TF), and Factor VIIa (TF:VIIa) activates Factor IX (FIX) and Factor X (FX) by limited proteolysis, thereby producing activated FIX (FIXa) and activated FX (FXa). The contact pathway initiates when Factor XIIa (FXII), prekallikrein (PK), and kininogen (HK) assemble on a surface or polymer. Assembly results in reciprocal activation of FXII to FXIIa by kallikrein (KK), and PK to KK by FXIIa. FXIIa then activates Factor XI (FXI) to FXIa, which then enzymatically converts FIX to FIXa. Activated FVIII (FVIIIa) combines with FIXa to form an Xase (FVIIIa:FIXa) complex responsible for the generation of FXa together with the initiating TF:FVIIa complex. Extrinsic and intrinsic pathways ultimately converge on the production of FXa, which leads to the activation of thrombin from prothrombin, thrombin-mediated cleavage of fibrinogen to fibrin, and activation, adhesion, and aggregation of platelets at the site of the clot.

VWF plays a critical role in platelet adhesion, activation, and aggregation during blood coagulation. Increased local shear stress near sites of vascular injury results in VWF binding to collagen in the sub-endothelial membrane, which in turn induces platelets to attach to tethered VWF by way of surface glycoprotein to initiate formation of the platelet plug. Under normal physiological conditions, the interaction between VWF and platelets occurs strictly in the context of vascular damage, since VWF does not interact with circulating platelets in the absence of injury. The A1 and C4 domains of VWF are essential for binding with platelet glycoproteins, such as, e.g., the platelet gp1b and gpIIbIIIa receptors, respectively. Because VWF and FVIII circulate as a complex in plasma under physiological conditions, accumulation of VWF at the site of vascular injury may increase the local concentration of FVIII. Similarly, reduced levels of VWF, as occurs, e.g., in VWD, may lead to reduced levels of FVIII.

Regulation of hemostasis is also critical in preventing excessive blood clotting, which can lead to the formation of thrombi that can dislodge from their site of formation and cause a potentially life-threatening embolism. Endothelial cells of intact blood vessels regulate clotting by way of thrombomodulin, an integral membrane protein, which reduces clotting by converting thrombin to an anti-coagulant form of the enzyme. At the same time, the thrombomodulin-thrombin complex can enzymatically activate the thrombin-activatable fibrinolysis inhibitor (TAFI) protein, a carboxypeptidase enzyme that reduces fibrinolysis by removing C-terminal lysine residues exposed on fibrin that are critical for the conversion of plasminogen to plasmin. TAFI's ability to attenuate fibrinolysis is highly dependent on its concentration, rate of activation, and blood plasma half-life.

Another factor important for the control of hemostasis is antithrombin (AT or AT3), a serine protease inhibitor (also known as serpin) that serves to regulate coagulation by inhibiting multiple proteases in the coagulation cascade, including FIXa, FXa, and thrombin. Its ability to inhibit coagulation factors is generally strongly enhanced by binding to heparin. By inhibiting proteins in the coagulation cascade, AT regulates the blood clotting process to prevent excessive clotting and thrombosis. In humans, AT deficiency is associated with an increased risk for thrombotic disease and pulmonary embolism.

### Anti-Von Willebrand Factor Antibodies

The mature VWF polypeptide is a large (2,050 amino acid residues) blood glycoprotein that functions in hemostasis and platelet adhesion. VWF is generally localized to the blood plasma, vascular endothelium, megakaryocytes, and subendothelial connective tissue, and exists in multimers of various sizes ranging from about 500 to 20,000 kDa. VWF multimer size typically scales with its role in hemostasis, i.e., larger VWF multimers are more effective at promoting clotting. Different domains of VWF are involved different functions. VWF contains the following domains, from N-terminus to C-terminus: D1-D2-D′-D3-A1-A2-A3-D4-C1-C2-C3-C4-C5-C6-CK. The D1-D2 domains, corresponding to the VWF propeptide, are proteolytically cleaved during processing of VWF into mature VWF. The D′-D3 domain mediates multimerization of VWF dimers and also binds to Factor VIII. The A1 domain binds to platelet gp1b receptor, heparin, and collagen. The A2 domain is cleaved by ADAMTS13, resulting in generation of smaller VWF multimers. The A3 domain binds to collagen. The C4 domain binds to platelet to gpIIa/IIIb. The CK domain is important for tail-to-tail homodimerization of VWF monomers (Springer, J Thromb Hemo 9:130-43 (2011)). Binding of VWF to other proteins, such as, e.g., FVIII, results in the stabilization of the binding partner and increases its blood plasma half-life. For example, in the absence of VWF, FVIII has a half-life of about 1-2 hours, but extends to 8-12 hours when bound by VWF. In addition to acting as a carrier of FVIII, VWF promotes clotting by facilitating platelet adhesion, activation, and aggregation. Deficiency or dysfunction of VWF is associated with a tendency to bleed and occurs in a number of blood disorders characterized by excessive bleeding, including, e.g., VWD and Heydes syndrome.

Provided herein, in certain embodiments, are antibodies that specifically bind to VWF proteins. In some embodiments, the VWF protein is a human VWF protein. In some embodiments, the human VWF protein has an amino acid sequence having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO: 1. In some embodiments, the human VWF protein has an amino acid sequence having at least 90% (e.g., at least 90%, 91%, 92%, 93%, 94%, 95%, 96%, 97%, 98%, 99%, or more) sequence identity to SEQ ID NO: 1. In some embodiments, the human VWF protein has an amino acid sequence having at least 95% (e.g., at least 95%, 96%, 97%, 98%, 99%, or more) sequence identity to SEQ ID NO: 1. In some embodiments, the human VWF protein has an amino acid sequence having at least 99% sequence identity to SEQ ID NO: 1. In some embodiments, the human VWF protein has an amino acid sequence of SEQ ID NO: 1.

MIPARFAGVLLALALILPGTLCAEGTRGRSSTARCSLFGSDFVNTFDGSM
YSFAGYCSYLLAGGCQKRSFSIIGDFQNGKRVSLSVYLGEFFDIHLFVNG
TVTQGDQRVSMPYASKGLYLETEAGYYKLSGEAYGFVARIDGSGNFQVLL
SDRYFNKTCGLCGNFNIFAEDDFMTQEGTLTSDPYDFANSWALSSGEQWC
ERASPPSSSCNISSGEMQKGLWEQCQLLKSTSVFARCHPLVDPEPFVALC
EKTLCECAGGLECACPALLEYARTCAQEGMVLYGWTDHSACSPVCPAGME
YRQCVSPCARTCQSLHINEMCQERCVDGCSCPEGQLLDEGLCVESTECPC
VHSGKRYPPGTSLSRDCNTCICRNSQWICSNEECPGECLVTGQSHFKSFD
NRYFTFSGICQYLLARDCQDHSFSIVIETVQCADDRDAVCTRSVTVRLPG
LHNSLVKLKHGAGVAMDGQDVQLPLLKGDLRIQHTVTASVRLSYGEDLQM
DWDGRGRLLVKLSPVYAGKTCGLCGNYNGNQGDDFLTPSGLAEPRVEDFG
NAWKLHGDCQDLQKQHSDPCALNPRMTRFSEEACAVLTSPTFEACHRAVS
PLPYLRNCRYDVCSCSDGRECLCGALASYAAACAGRGVRVAWREPGRCEL
NCPKGQVYLQCGTPCNLTCRSLSYPDEECNEACLEGCFCPPGLYMDERGD
CVPKAQCPCYYDGEIFQPEDIFSDHHTMCYCEDGFMHCTMSGVPGSLLPD
AVLSSPLSHRSKRSLSCRPPMVKLVCPADNLRAEGLECTKTCQNYDLECM
SMGCVSGCLCPPGMVRHENRCVALERCPCFHQGKEYAPGETVKIGCNTCV
CQDRKWNCTDHVCDATCSTIGMAHYLTFDGLKYLFPGECQYVLVQDYCGS
NPGTFRILVGNKGCSHPSVKCKKRVTILVEGGEIELFDGEVNVKRPMKDE
THFEVVESGRYIILLLGKALSVVWDRHLSISVVLKQTYQEKVCGLCGNFD
GIQNNDLTSSNLQVEEDPVDFGNSWKVSSQCADTRKVPLDSSPATCHNNI
MKQTMVDSSCRILTSDVFQDCNKLVDPEPYLDVCIYDTCSCESIGDCACF
CDTIAAYAHVCAQHGKVVTWRTATLCPQSCEERNLRENGYECEWRYNSCA
PACQVTCQHPEPLACPVQCVEGCHAHCPPGKILDELLQTCVDPEDCPVCE
VAGRRFASGKKVTLNPSDPEHCQICHCDVVNLTCEACQEPGGLVVPPTDA
PVSPTTLYVEDISEPPLHDFYCSRLLDLVFLLDGSSRLSEAEFEVLKAFV
VDMMERLRISQKWVRVAVVEYHDGSHAYIGLKDRKRPSELRRIASQVKYA
GSQVASTSEVLKYTLFQIFSKIDRPEASRITLLLMASQEPQRMSRNFVRY
VQGLKKKKVIVIPVGIGPHANLKQIRLIEKQAPENKAFVLSSVDELEQQR
DEIVSYLCDLAPEAPPPTLPPDMAQVTVGPGLLGVSTLGPKRNSMVLDVA
FVLEGSDKIGEADFNRSKEFMEEVIQRMDVGQDSIHVTVLQYSYMVTVEY
PFSEAQSKGDILQRVREIRYQGGNRTNTGLALRYLSDHSFLVSQGDREQA
PNLVYMVTGNPASDEIKRLPGDIQVVPIGVGPNANVQELERIGWPNAPIL
IQDFETLPREAPDLVLQRCCSGEGLQIPTLSPAPDCSQPLDVILLLDGSS
SFPASYFDEMKSFAKAFISKANIGPRLTQVSVLQYGSITTIDVPWNVVPE
KAHLLSLVDVMQREGGPSQIGDALGFAVRYLTSEMHGARPGASKAVVILV
TDVSVDSVDAAADAARSNRVTVFPIGIGDRYDAAQLRILAGPAGDSNVVK
LQRIEDLPTMVTLGNSFLHKLCSGFVRICMDEDGNEKRPGDVWTLPDQCH
TVTCQPDGQTLLKSHRVNCDRGLRPSCPNSQSPVKVEETCGCRWTCPCVC
TGSSTRHIVTFDGQNFKLTGSCSYVLFQNKEQDLEVILHNGACSPGARQG
CMKSIEVKHSALSVELHSDMEVTVNGRLVSVPYVGGNMEVNVYGAIMHEV
RFNHLGHIFTFTPQNNEFQLQLSPKTFASKTYGLCGICDENGANDFMLRD
GTVTTDWKTLVQEWTVQRPGQTCQPILEEQCLVPDSSHCQVLLLPLFAEC
HKVLAPATFYAICQQDSCHQEQVCEVIASYAHLCRTNGVCVDWRTPDFCA
MSCPPSLVYNHCEHGCPRHCDGNVSSCGDHPSEGCFCPPDKVMLEGSCVP
EEACTQCIGEDGVQHQFLEAWVPDHQPCQICTCLSGRKVNCTTQPCPTAK
APTCGLCEVARLRQNADQCCPEYECVCDPVSCDLPPVPHCERGLQPTLTN
PGECRPNFTCACRKEECKRVSPPSCPPHRLPTLRKTQCCDEYECACNCVN
STVSCPLGYLASTATNDCGCTTTTCLPDKVCVHRSTIYPVGQFWEEGCDV
CTCTDMEDAVMGLRVAQCSQKPCEDSCRSGFTYVLHEGECCGRCLPSACE
VVTGSPRGDSQSSWKSVGSQWASPENPCLINECVRVKEEVFIQQRNVSCP
QLEVPVCPSGFQLSCKTSACCPSCRCERMEACMLNGTVIGPGKTVMIDVC
TTCRCMVQVGVISGFKLECRKTTCNPCPLGYKEENNTGECCGRCLPTACT
IQLRGGQIMTLKRDETLQDGCDTHFCKVNERGEYFWEKRVTGCPPFDEHK
CLAEGGKIMKIPGTCCDTCEEPECNDITARLQYVKVGSCKSEVEVDIHYC
QGKCASKAMYSIDINDVQDQCSCCSPTRTEPMQVALHCTNGSVVYHEVLN
AMECKCSPRKCSK (SEQ ID NO: 1; UniProt No. P04275-1)

In some embodiments, the VWF protein is a VWF protein of a non-human primate (NHP; e.g., Macaca fascicularis). In some embodiments, the NHP VWF protein has an amino acid sequence having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO: 2. In some embodiments, the NHP VWF protein has an amino acid sequence having at least 90% (e.g., at least 90%, 91%, 92%, 93%, 94%, 95%, 96%, 97%, 98%, 99%, or more) sequence identity to SEQ ID NO: 2. In some embodiments, the NHP VWF protein has an amino acid sequence having at least 95% (e.g., at least 95%, 96%, 97%, 98%, 99%, or more) sequence identity to SEQ ID NO: 2. In some embodiments, the NHP VWF protein has an amino acid sequence having at least 99% sequence identity to SEQ ID NO: 2. In some embodiments, the NHP VWF protein has an amino acid sequence of SEQ ID NO: 2.

MIPARLAGVLLALALVLPGTLCAEGTRGRSSMARCSLFGSDFINTFDGSM
YSFAGYCSYLLAGDCQKRSFSIIGDFQNGKRVSLSVYLGEFFDIHLFVNG
TVIQGDKSVSMPYASKGLYLETEAGYYKLSGEAYGFVARIDGSGNFQVLL
SDRYFNKTCGLCGNFNIFAEDDFMTQEGTVTSDPYDFANSWALSSGEQWC
ERASPPSSSCNISSGEVQKGLWEQCQLLKSTSVFARCHPLVDPEPFVALC
EKTSCECAGGLECTCPAFLEYTRTCAQEGMVLYGWTDHSACSPVCPAGME
YKQCVSPCARTCQSLHINEVCQERCVDGCSCPEGQLLDEGLCVESTECPC
MHSGKRYPPGASLSRDCNTCICRNSQWICSNEECPGECLVTGQSHFKSFD
NRYFTFSGICQYLLARDCQDHSFSIVIETVQCADDPDAVCTRSVTVRLPG
LHNSLVKLKHGGGVAMDGQDVQLPLLKGDLRIQHSVTASVRLSYGEDLQM
DWDGRGRLLVKLSPVYAGKTCGLCGNYNGNQGDDFLTPSGLAEPRVEDFG
NAWKLHADCQDLQKQHSDPCALNPRMTRFSEEACAVLTSPTFEACHRAVS
PLPYLRNCRYDVCSCSDGRECLCGALASYAACAGGVVAWEPGRCELKCPK
GQVYLQCGTPCNLTCRALSYPDEECSEACLEGCFCPPGLYMDEMGDCVPK
AQCPCYYDGEIFQPEDIFSDHHTMCYCEDGFMHCTMSGVPGSLLPDAVLS
SPLSHRSKRSLSCRPPMVKLVCPADNPRAEGLECAKTCQNYDLECMSMGC
VSGCLCPPGMVRHENRCVALERCPCLHQGKEYAPGEAVKIDCNTCVCRDR
KWNCTDHVCDATCSMIGMAHYLTFDGLKYMFPGECQYVLVQDYCGGNPGT
FRILVGNEGCSHPSVKCKKHVTILVEGGEIELFDGEVNVKRPMKDETHFE
VVESGRYIILLLGKAISVVWDRHLSISVVLKQTYQEKVCGLCGNFDGIQN
NDLTSSNLQVEEDPVDFGNSWKVSSQCADTRKVPLDSSPATCHNNLMKQT
MVDSSCRILTSDVFQDCNKLVDPEPFLDVCIYDTCSCESIGDCACFCDTI
AAYAHVCAQHGKVVTWRTATLCPQSCEERNLRENGYECEWRYNSCAPACR
VTCQHPEPLACPVQCVEGCHAHCPPGKILDELLQTCVSPEDCPVCEAAGR
RFASGKKVTLNPSDPEHCQICHCDGVNLTCEACEEPGGLVVPPTDAPVSP
TTPYVEDISEPPLHDFYCSRLLDLVFLLDGSSRLSEAEFEVLKAFVVDMM
ERLRISQKWVRVAVVEYHDGSHAYIGLKDRKRPSELRRIASQVKYAGSQV
ASTSEVLKYTLFQIFGKIDRPEASRIALLLMASQEPQRMSRNFVRYVQGL
KKKKVIVIPVGIGPHANLKQIRLIEKQSPENKAFVLSGVDELEQQRDEIV
SYLCDLAPEAPPPTLPPDMAQVTVGSGLLGVSTLGPKRNSMVLDVAFVLE
GSDKIGEADFNRSKEFMEEVIQRMDVGQDGIHVTVLQYSYTVAVEYPFSE
AQSKGDILQRVREIRYQGGNRTNTGLALQYLSDHSFLVSQGDREQAPNLV
YMVTGNPASDEIKRLPGDIQVVPIGVGPHANVQELERIGWPNAPILIQDF
ETLPREAPDLVLQSCCSGEGLKIPTLSPAPDCSQPLDVILLLDGSSSFPA
AYFDEMKSFAKAFISKANIGPHLTQVSVLQYGSITTIDVPWNVAPEKAHL
LSLVDVMQREGGPSQIGGALGFAVRYLTSEMHGARPGASKAVVILVMDVS
VDAVDAAADAARSNRVAVFPIGIGDRYDAAQLRILAGPAGNSNVVKLQRI
EDLPTMVTLGNSFLHKLCSGFVRICMDEDGNERRPGDIWTLPDQCHTVTC
QPDGQTLLESHRVNCDRGLRPSCPNSQSPVKVEETCGCRWTCPCVCTGSS
TRHIVTFDGQNFKLTGSCSYVLFQNKEQDLEVILHNGACSPAARQGCMKS
IEVKHAALSVELHSDMEVTVNGRLVSVPYVGGNMEVNVYGAIMHEIRFNH
LGHIFTFTPQNNEFQLQLSPKTFASKTYGLCGICDENGANDFMLRDGTVT
TDWKTLVQEWTVQRPGQTCQPILEEQCLVPNSSQCQVLLSALFAECHKVL
APATFYAICQQDSCHREQVCEVIASYAHLCRTNGVCVDWRTPDFCAMSCP
PSLVYNPCERGCPRHCNGNVSSCGDHPSEGCFCPPNKVMLEGSCVPEEAC
TQCIGEDGVQHQFLEAWVPDHQPCQICTCLSGRKANCTMQPCPTAKAPTC
GLCEVARLRQNADQCCPEYECVCDPESCDLPPVPHCEGGLQPTLTNPGEC
RPNFTCACRKEECKRVSLPSCPPHRLPTLRKTQCCDEYECACNCVNSTVS
CPLGYLASTATNDCGCTTTTCLPDKVCVHRSTIYPVGQFWEEGCDVCTCT
DMEDAVMGLRVVQCSQKPCEDSCRSGFTYVPREGECCGRCLPSACEVVTG
SPRGDSQSSWKSVGSHWASPENPCLINECVRVKEEVFVQQRNVSCPQLEV
PVCPSGFQLSCKTSACCPSCRCEPVEACMLNGTMIGPGKSVMIDACTTCR
CIVQVGSISGFKLECRKTICNPCPLGYKEEKNTGECCGRCLPTVCTIRLR
GGQIMTLKRDETLQDGCDTHFCKVNERGEYFWEKRVTGCPPFDEHKCLAE
GGKIKKIPGTCCDTCEEPECSDITARLQYVKVGSCKSEVEVDIHYCQGKC
ASKAMYSIDINDVQDQCSCCSPTRTEPMQVPLHCTNGSVVYHEVLNAMQC
ECSPRKCSK (SEQ ID NO: 2; UniProt No. A0A2K5X4G5)

In some embodiments, the VWF protein is a VWF protein of a canine (e.g., Canis familiaris). In some embodiments, the canine VWF protein has an amino acid sequence having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO: 3. In some embodiments, the canine VWF protein has an amino acid sequence having at least 90% (e.g., at least 90%, 91%, 92%, 93%, 94%, 95%, 96%, 97%, 98%, 99%, or more) sequence identity to SEQ ID NO: 3. In some embodiments, the canine VWF protein has an amino acid sequence having at least 95% (e.g., at least 95%, 96%, 97%, 98%, 99%, or more) sequence identity to SEQ ID NO: 3. In some embodiments, the canine VWF protein has an amino acid sequence having at least 99% sequence identity to SEQ ID NO: 3. In some embodiments, the canine VWF protein has an amino acid sequence of SEQ ID NO: 3.

MSPTRLVRVLLALALILPGKLCTKGTVGRSSMARCSLFGGDFINTFDESM
YSFAGDCSYLLAGDCQEHSVSLIGGFQNGKRVSLSVYLGEFFDIHLFVNG
TMLQGTQSISMPYASNGLYLEAEAGYYKLSSEAYGFVARIDGNGNFQVLL
SDRYFNKTCGLCGNFNIFAEDDFRTQEGTLTSDPYDFANSWALSSGEQRC
KRVSPPSSPCNVSSDEVQQVLWEQCQLLKSASVFARCHPLVDPEPFVALC
ERTLCTCVQGMECPCAVLLEYARACAQQGIVLYGWTDHSVCRPACPAGME
YKECVSPCTRTCQSLHVKEVCQEQCVDGCSCPEGQLLDEGHCVGSAECSC
VHAGQRYPPGASLLQDCHTCICRNSLWICSNEECPGECLVTGQSHFKSFD
NRYFTFSGVCHYLLAQDCQDHTFSVVIETVQCADDLDAVCTRSVTVRLPG
HHNSLVKLKHGGGVSMDGQDIQIPLLQGDLRIQHTVMASVRLSYGEDLQM
DWDGRGRLLVTLSPAYAGKTCGLCGNYNGNRGDDFVTPAGLAEPLVEDFG
NAWKLLGACENLQKQHRDPCSLNPRQARFAEEACALLTSSKFEPCHRAVG
PQPYVQNCRYDVCSCSDGRDCLCSAVANYAAACARRGVHIAWREPGFCAL
SCPQGQVYLQCGTPCNMTCRSLSYPEEDCNEVCLEGCFCPPGLYLDERGD
CVPKAQCPCYYDGEIFQPEDIFSDHHTMCYCEDGFMHCTTSGGLGSLLPN
PVLSSPRSHRSKRSLSCRPPMVKLVCPADNPRAEGLECAKTCQNYDLQCM
STGCVSGCLCPQGMVRHENRCVALERCPCFHQGQEYAPGETVKIDCNTCV
CRDRKWNCTDHVCDATCSAIGMAHYLTFDGLKYLFPGECQYVLVQDYCGS
NPGTFRILVGNEGCSYPSVKCKKRVTILVEGGEIELFDGEVNVKKPMKDE
THFEVVESGQYVILLLGKALSVVWDHRLSISVTLKRTYQEQVCGLCGNFD
GIQNNDFTSSSLQIEEDPVDFGNSWKVNPQCADTKKVPLDSSPAVCHNNI
MKQTMVDSSCRILTSDIFQDCNRLVDPEPFLDICIYDTCSCESIGDCTCF
CDTIAAYAHVCAQHGKVVAWRTATFCPQNCEERNLHENGYECEWRYNSCA
PACPITCQHPEPLACPVQCVEGCHAHCPPGKILDELLQTCIDPEDCPVCE
VAGRRLAPGKKIILNPSDPEHCQICHCDGVNFTCQACREPGSLVVPPTEG
PIGSTTSYVEDTPEPPLHDFHCSRLLDLVFLLDGSSKLSEDEFEVLKVFV
VGMMEHLHISQKRIRVAVVEYHDGSHAYIELKDRKRPSELRRITSQVKYA
GSEVASTSEVLKYTLFQIFGKIDRPEASRIALLLMASQEPSRLARNLVRY
VQGLKKKKVIVIPVGIGPHASLKQIHLIEKQAPENKAFVFSGVDELEQRR
DEIINYLCDLAPEAPAPTQHPPMAQVTVGSELLGVSSPGPKRNSMVLDVV
FVLEGSDKIGEANFNKSREFMEEVIQRMDVGQDRIHVTVLQYSYMVTVEY
TFSEAQSKGEVLQQVRDIRYRGGNRTNTGLALQYLSEHSFSVSQGDREQV
PNLVYMVTGNPASDEIKRMPGDIQVVPIGVGPHANVQELEKIGWPNAPIL
IHDFEMLPREAPDLVLQRCCSGEGLQIPTLSPTPDCSQPLDVVLLLDGSS
SIPASYFDEMKSFTKAFISRANIGPRLTQVSVLQYGSITTIDVPWNVAYE
KVHLLSLVDLMQQEGGPSQIGDALSFAVRYVTSEVHGARPGASKAVVILV
TDVSVDSVDAAAEAARSNRVTVFPIGIGDRYSEAQLSSLAGPKAGSNMVR
LQRIEDLPTVATLGNSFFHKLCSGFDRVCVDEDGNEKRPGDVWTLPDQCH
TVTCLPDGQTLLKSHRVNCDRGPRPSCPNGQPPLRVEETCGCRWTCPCVC
MGSSTRHIVTFDGQNFKLTGSCSYVLFQNKEQDLEVILHNGACSPGAKET
CMKSIEVKHDGLSVELHSDMQMTVNGRLVSIPYVGGDMEVNVYGTIMYEV
RFNHLGHIFTFTPQNNEFQLQLSPRTFASKTYGLCGICDENGANDFILRD
GTVTTDWKALIQEWTVQQLGKTCQPVPEEQCPVSSSSHCQVLLSELFAEC
HKVLAPATFYAMCQPDSCHPKKVCEAIALYAHLCRTKGVCVDWRRANFCA
MSCPPSLVYNHCEHGCPRLCEGNTSSCGDQPSEGCFCPPNQVMLEGSCVP
EEACTQCISEDGVRHQFLETWVPAHQPCQICTCLSGRKVNCTLQPCPTAR
APTCGPCEVARLRQNAEQCCPEYECVCDLVSCDLPPVPPCEDGLQMTLTN
PGECRPNFTCACRKDECRRESPPSCPPHRTLALRKTQCCDEYECACNCVN
STVSCPLGYLASAVTNDCGCTTTTCFPDKVCVHRGTIYPVGQFWEEACDV
CTCTDLEDSVMGLRVAQCSQKPCEDNCLSGFTYVLHEGECCGRCLPSACE
VVIGSPRGDAQSHWKNVGSHWASPDNPCLINECVRVKEEVFVQQRNVSCP
QLNVPTCPTGFQLSCKTSECCPTCHCEPLEACLLNGTIIGPGKSLMIDVC
TTCRCTVQVGVISGFKLECRKTTCEACPLGYKEEKNQGECCGRCLPIACT
IQLRGGQIMTLKRDETIQDGCDSHFCKVNERGEYIWEKRVTGCPPFDEHK
CLAEGGKIMKIPGTCCDTCEEPECKDIIAKLQRVKVGDCKSEEEVDIHYC
EGKCASKAVYSIHMEDVQDQCSCCSPTQTEPMQVPLRCTNGSLIYHEILN
AMQCRCSPRKCSK (SEQ ID NO: 3; UniProt ID No.
Q28295-1)

In some embodiments, the VWF protein is a VWF protein of a mouse (e.g., Mus musculus). In some embodiments, the murine VWF protein has an amino acid sequence having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO: 4. In some embodiments, the murine VWF protein has an amino acid sequence having at least 90% (e.g., at least 90%, 91%, 92%, 93%, 94%, 95%, 96%, 97%, 98%, 99%, or more) sequence identity to SEQ ID NO: 4. In some embodiments, the murine VWF protein has an amino acid sequence having at least 95% (e.g., at least 95%, 96%, 97%, 98%, 99%, or more) sequence identity to SEQ ID NO: 4. In some embodiments, the murine VWF protein has an amino acid sequence having at least 99% sequence identity to SEQ ID NO: 4. In some embodiments, the murine VWF protein has an amino acid sequence of SEQ ID NO: 4.

(SEQ ID NO: 4; UniProt ID No. Q8CIZ8-1)
MNPFRYEICLLVLALTWPGTLCTEKPRDRPSTARCSLFGDDFINTFDETMYSFAGGCS
YLLAGDCQKRSFSILGNFQDGKRMSLSVYLGEFFDIHLFANGTVMQGDQSISMPYAS
QGLYLELEAGYYKLSSETFGFAARIDGNGNFQVLMSDRHFNKTCGLCGDFNIFAEDD
FRTQEGTLTSDPYDFANSWALSSEEQRCKRASPPSRNCESSSGDMHQAMWEQCQLL
KTASVFARCHPLVDPESFVALCEKILCTCATGPECACPVLLEYARTCAQEGMVLYG
WTDHSACRPACPAGMEYKECVSPCPRTCQSLSINEVCQQQCVDGCSCPEGELLDED
RCVQSSDCPCVHAGKRYPPGTSLSQDCNTCICRNSLWICSNEECPGECLVTGQSHFKS
FDNRYFTFSGICQYLLARDCEDHTFSIVIETMQCADDPDAVCTRSVSVRLSALHNSLV
KLKHGGAVGIDGQDVQLPFLQGDLRIQHTVMASVRLSYAEDLQMDWDGRGRLLVK
LSPVYSGKTCGLCGNYNGNKGDDFLTPAGLVEPLVVDFGNAWKLQGDCSDLRRQH
SDPCSLNPRLTRFAEEACALLTSSKFEACHHAVSPLPYLQNCRYDVCSCSDSRDCLCN
AVANYAAECARKGVHIGWREPGFCALGCPQGQVYLQCGNSCNLTCRSLSLPDEECS
EVCLEGCYCPPGLYQDERGDCVPKAQCPCYYDGELFQPADIFSDHHTMCYCEDGFM
HCTTSGTLGSLLPDTVLSSPLSHRSKRSLSCRPPMVKLVCPADNPRAQGLECAKTCQ
NYDLERMSLGCVSGCLCPPGMVRHENKCVALERCPCFHQGAEYAPGDTVKIGCNTC
VCRERKWNCTNHVCDATRSAIGMAHYLTFDGLKYLFPGECQYVLVYDYCGSNPGT
FQILVGNEGCSYPSVKCRKRVTILVDGGELELFDGEVNVKRPLRDESHFEVVESGRY
VILLLGQALSVVWDHHLSISVVLKHTYQEQVCGLCGNFDGIQNNDSTTSSLQVEEDP
VNFGNSWKVSSQCADTRKLSLDVSPATCHNNIMKQTMVDSACRILTSDVFQGCNRL
VDPEPYLDICIYDTCSCESIGDCACFCDTIAAYAHVCAQHGQVVAWRTPTLCPQSCEE
KNVRENGYECEWRYNSCAPACPVTCQHPEPLACPVQCVEGCHAHCPPGRILDELLQ
TCVDPQDCPVCEVAGRRLAPGKKITLSPDDPAHCQNCHCDGVNLTCEACQEPGGLV
APPTDAPVSSTTPYVEDTPEPPLHNFYCSKLLDLVFLLDGSSMLSEAEFEVLKAFVVG
MMERLHISQKRIRVAVVEYHDGSRAYLELKARKRPSELRRITSQIKYTGSQVASTSEV
LKYTLFQIFGKIDRPEASHITLLLTASQEPPRMARNLVRYVQGLKKKKVIVIPVGIGPH
ASLKQIRLIEKQAPENKAFLLSGVDELEQRRDEIVSYLCDLAPEAPAPTQPPQVAHVT
VSPGIAGISSPGPKRKSMVLDVVFVLEGSDEVGEANFNKSKEFVEEVIQRMDVSPDA
TRISVLQYSYTVTMEYAFNGAQSKEEVLRHVREIRYQGGNRTNTGQALQYLSEHSFS
PSQGDRVEAPNLVYMVTGNPASDEIKRLPGDIQVVPIGVGPHANMQELERISRPIAPIF
IRDFETLPREAPDLVLQTCCSKEGLQLPTLPPLPDCSQPLDVVLLLDGSSSLPESSFDK
MKSFAKAFISKANIGPHLTQVSVIQYGSINTIDVPWNVVQEKAHLQSLVDLMQQEGG
PSQIGDALAFAVRYVTSQIHGARPGASKAVVIIIMDTSLDPVDTAADAARSNRVAVFP
VGVGDRYDEAQLRILAGPGASSNVVKLQQVEDLSTMATLGNSFFHKLCSGFSGVCV
DEDGNEKRPGDVWTLPDQCHTVTCLANGQTLLQSHRVNCDHGPRPSCANSQSPVR
VEETCGCRWTCPCVCTGSSTRHIVTFDGQNFKLTGSCSYVIFQNKEQDLEVLLHNGA
CSPGAKQACMKSIEIKHAGVSAELHSNMEMAVDGRLVLAPYVGENMEVSIYGAIMY
EVRFTHLGHILTYTPQNNEFQLQLSPKTFASKMHGLCGICDENGANDFTLRDGTVTT
DWKRLVQEWTVQQPGYTCQAVPEEQCPVSDSSHCQVLLSASFAECHKVIAPATFHTI
CQQDSCHQERVCEVIASYAHLCRTSGVCVDWRTTDFCAMSCPPSLVYNHCERGCPR
HCDGNTSFCGDHPSEGCFCPQHQVFLEGSCVPEEACTQCVGEDGVRHQFLETWVPD
HQPCQICMCLSGRKINCTAQPCPTARAPTCGPCEVARLKQSTNLCCPEYECVCDLEN
CNLPPVPPCEGGLQPTLTNPGECRPTFTCDCRKEECKRVSPPSCPPHRTPTLRKTQCC
DEYECACSCVNSTLSCPLGYLASATTNDCGCTTTTCLPDKVCVHRGTVYPVGQFWE
EGCDTCTCTDMEDTVVGLRVVQCSQRPCEDSCQPGFSYVLHEGECCGRCLPSACKV
VAGSLRGDSHSSWKSVGSRWAVPENPCLVNECVRVEDAVFVQQRNISCPQLAVPTC
PTGFQLNCETSECCPSCHCEPVEACLLNGTIIGPGKSVMVDLCTTCRCIVQTDAISRFK
LECRKTTCEACPMGYREEKSQGECCGRCLPTACTIQLRGGRIMTLKQDETFQDGCDS
HLCRVNERGEYIWEKRVTGCPPFDEHKCLAEGGKIVKIPGTCCDTCEEPDCKDITAK
VQYIKVGDCKSQEEVDIHYCQGKCASKAVYSIDIEDVQEQCSCCLPSRTEPMRVPLH
CTNGSVVYHEVINAMQCRCSPRNCSK

### Antibody Sequences

In some embodiments, the antibody comprises a complementarity determining region (CDR) H1 having an amino acid sequence 100% identical to the amino acid sequence of SEQ ID NO: 13, a CDR H2 having an amino acid sequence 100% identical to the amino acid sequence of SEQ ID NO: 14, and a CDR H3 having an amino acid sequence 100% identical to the amino acid sequence of SEQ ID NO: 15. In some embodiments, the antibody comprises a complementarity determining region (CDR) L1 having 100% sequence identity to an amino acid sequence of SEQ ID NO: 17, a CDR L2 having an amino acid sequence 100% identical to the amino acid sequence of KAS, and a CDR L3 having an amino acid sequence 100% identical to the amino acid sequence of SEQ ID NO: 18. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 12. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 16. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 12. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 16. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 12. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 16. In some embodiments, the antibody comprises a variable heavy chain (VH) region having an amino acid sequence with at least 95% (e.g., at least 95%, 96%, 97%, 98%, 99%, or more) identity to SEQ ID NO: 12. In some embodiments, the antibody comprises a variable light chain (VL) region having an amino acid sequence with at least 95% (e.g., at least 95%, 96%, 97%, 98%, 99%, or more) identity to SEQ ID NO: 16. In some embodiments, the antibody comprises a variable heavy chain (VH) region having an amino acid sequence 100% identical with the amino acid sequence of SEQ ID NO: 12. In some embodiments, the antibody comprises a variable light chain (VL) region having an amino acid sequence 100% identical with the amino acid sequence of SEQ ID NO: 16. In some embodiments, the antibody comprises: (a) a complementarity determining region (CDR) H1 having an amino acid sequence 100% identical to the amino acid sequence of SEQ ID NO: 13, a CDR H2 having an amino acid sequence 100% identical to the amino acid sequence of SEQ ID NO: 14, and a CDR H3 having an amino acid sequence 100% identical to the amino acid sequence of SEQ ID NO: 15; and (b) a complementarity determining region (CDR) L1 having 100% sequence identity to an amino acid sequence of SEQ ID NO: 17, a CDR L2 having an amino acid sequence 100% identical to the amino acid sequence of KAS, and a CDR L3 having an amino acid sequence 100% identical to the amino acid sequence of SEQ ID NO: 18. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 12 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 16. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 12 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 16. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 12 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 16. In some embodiments, the antibody comprises a variable heavy chain (VH) region having an amino acid sequence with at least 95% (e.g., at least 95%, 96%, 97%, 98%, 99%, or more) identity to SEQ ID NO: 12 and a variable light chain (VL) region having an amino acid sequence with at least 95% (e.g., at least 95%, 96%, 97%, 98%, 99%, or more) identity to SEQ ID NO: 16. In some embodiments, the antibody comprises a variable heavy chain (VH) region having an amino acid sequence 100% identical with the amino acid sequence of SEQ ID NO: 12 and a variable light chain (VL) region having an amino acid sequence 100% identical with the amino acid sequence of SEQ ID NO: 16. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 12 and comprising therein a complementarity determining region (CDR) H1 having an amino acid sequence 100% identical to the amino acid sequence of SEQ ID NO: 13, a CDR H2 having an amino acid sequence 100% identical to the amino acid sequence of SEQ ID NO: 14, and a CDR H3 having an amino acid sequence 100% identical to the amino acid sequence of SEQ ID NO: 15. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 16 and comprising therein a complementarity determining region (CDR) L1 having 100% sequence identity to an amino acid sequence of SEQ ID NO: 17, a CDR L2 having an amino acid sequence 100% identical to the amino acid sequence of KAS, and a CDR L3 having an amino acid sequence 100% identical to the amino acid sequence of SEQ ID NO: 18. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 12 and comprising therein a complementarity determining region (CDR) H1 having an amino acid sequence 100% identical to the amino acid sequence of SEQ ID NO: 13, a CDR H2 having an amino acid sequence 100% identical to the amino acid sequence of SEQ ID NO: 14, and a CDR H3 having an amino acid sequence 100% identical to the amino acid sequence of SEQ ID NO: 15; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 16 and comprising therein a complementarity determining region (CDR) L1 having 100% sequence identity to an amino acid sequence of SEQ ID NO: 17, a CDR L2 having an amino acid sequence 100% identical to the amino acid sequence of KAS, and a CDR L3 having an amino acid sequence 100% identical to the amino acid sequence of SEQ ID NO: 18. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 453. In some embodiments, the antibody comprises a second heavy chain region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 473. In some embodiments, the antibody comprises a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 463. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 453, a second heavy chain region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 473, and a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 463.

In some embodiments, the antibody comprises a complementarity determining region (CDR) H1 having an amino acid sequence 100% identical to the amino acid sequence of SEQ ID NO: 20, a CDR H2 having an amino acid sequence 100% identical to the amino acid sequence of SEQ ID NO: 21, and a CDR H3 having an amino acid sequence 100% identical to the amino acid sequence of SEQ ID NO: 22. In some embodiments, the antibody comprises a complementarity determining region (CDR) L1 having 100% sequence identity to an amino acid sequence of SEQ ID NO: 24, a CDR L2 having an amino acid sequence 100% identical to the amino acid sequence of AAS, and a CDR L3 having an amino acid sequence 100% identical to the amino acid sequence of SEQ ID NO: 25. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 19. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 23. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 19. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 23. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 19. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 23. In some embodiments, the antibody comprises a variable heavy chain (VH) region having an amino acid sequence with at least 95% (e.g., at least 95%, 96%, 97%, 98%, 99%, or more) identity to SEQ ID NO: 19. In some embodiments, the antibody comprises a variable light chain (VL) region having an amino acid sequence with at least 95% (e.g., at least 95%, 96%, 97%, 98%, 99%, or more) identity to SEQ ID NO: 23. In some embodiments, the antibody comprises a variable heavy chain (VH) region having an amino acid sequence 100% identical with the amino acid sequence of SEQ ID NO: 19. In some embodiments, the antibody comprises a variable light chain (VL) region having an amino acid sequence 100% identical with the amino acid sequence of SEQ ID NO: 23. In some embodiments, the antibody comprises: (a) a complementarity determining region (CDR) H1 having an amino acid sequence 100% identical to the amino acid sequence of SEQ ID NO: 20, a CDR H2 having an amino acid sequence 100% identical to the amino acid sequence of SEQ ID NO: 21, and a CDR H3 having an amino acid sequence 100% identical to the amino acid sequence of SEQ ID NO: 22; and (b) a complementarity determining region (CDR) L1 having 100% sequence identity to an amino acid sequence of SEQ ID NO: 24, a CDR L2 having an amino acid sequence 100% identical to the amino acid sequence of AAS, and a CDR L3 having an amino acid sequence 100% identical to the amino acid sequence of SEQ ID NO: 25. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 19 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 23. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 19 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 23. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 19 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 23. In some embodiments, the antibody comprises a variable heavy chain (VH) region having an amino acid sequence with at least 95% (e.g., at least 95%, 96%, 97%, 98%, 99%, or more) identity to SEQ ID NO: 19 and a variable light chain (VL) region having an amino acid sequence with at least 95% (e.g., at least 95%, 96%, 97%, 98%, 99%, or more) identity to SEQ ID NO: 23. In some embodiments, the antibody comprises a variable heavy chain (VH) region having an amino acid sequence 100% identical with the amino acid sequence of SEQ ID NO: 19 and a variable light chain (VL) region having an amino acid sequence 100% identical with the amino acid sequence of SEQ ID NO: 23. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 19 and comprising therein a complementarity determining region (CDR) H1 having an amino acid sequence 100% identical to the amino acid sequence of SEQ ID NO: 20, a CDR H2 having an amino acid sequence 100% identical to the amino acid sequence of SEQ ID NO: 21, and a CDR H3 having an amino acid sequence 100% identical to the amino acid sequence of SEQ ID NO: 22. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 23 and comprising therein a complementarity determining region (CDR) L1 having 100% sequence identity to an amino acid sequence of SEQ ID NO: 24, a CDR L2 having an amino acid sequence 100% identical to the amino acid sequence of AAS, and a CDR L3 having an amino acid sequence 100% identical to the amino acid sequence of SEQ ID NO: 25. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 19 and comprising therein a complementarity determining region (CDR) H1 having an amino acid sequence 100% identical to the amino acid sequence of SEQ ID NO: 20, a CDR H2 having an amino acid sequence 100% identical to the amino acid sequence of SEQ ID NO: 21, and a CDR H3 having an amino acid sequence 100% identical to the amino acid sequence of SEQ ID NO: 22; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 23 and comprising therein a complementarity determining region (CDR) L1 having 100% sequence identity to an amino acid sequence of SEQ ID NO: 24, a CDR L2 having an amino acid sequence 100% identical to the amino acid sequence of AAS, and a CDR L3 having an amino acid sequence 100% identical to the amino acid sequence of SEQ ID NO: 25. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 454. In some embodiments, the antibody comprises a second heavy chain region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 474. In some embodiments, the antibody comprises a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 464. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 454, a second heavy chain region comprising an amino acid sequence having at least 80% (e.g., at least 80%, 81%, 82%, 83%, 84%, 85%, 86%, 87%, 88%, 89%0, 90%, 91%, 92%, 93%, 94%, 95%, 96%, 97%, 98%, 99%, or more) identity to SEQ ID NO: 474, and a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 464.

In some embodiments, the antibody comprises a complementarity determining region (CDR) H1 having an amino acid sequence 100% identical to the amino acid sequence of SEQ ID NO: 27, a CDR H2 having an amino acid sequence 100% identical to the amino acid sequence of SEQ ID NO: 28, and a CDR H3 having an amino acid sequence 100% identical to the amino acid sequence of SEQ ID NO: 29. In some embodiments, the antibody comprises a complementarity determining region (CDR) L1 having 100% sequence identity to an amino acid sequence of SEQ ID NO: 31, a CDR L2 having an amino acid sequence 100% identical to the amino acid sequence of ESS, and a CDR L3 having an amino acid sequence 100% identical to the amino acid sequence of SEQ ID NO: 32. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 26. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 30. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 26. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 30. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 26. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 30. In some embodiments, the antibody comprises a variable heavy chain (VH) region having an amino acid sequence with at least 95% (e.g., at least 95%, 96%, 97%, 98%, 99%, or more) identity to SEQ ID NO: 26. In some embodiments, the antibody comprises a variable light chain (VL) region having an amino acid sequence with at least 95% (e.g., at least 95%, 96%, 97%, 98%, 99%, or more) identity to SEQ ID NO: 30. In some embodiments, the antibody comprises a variable heavy chain (VH) region having an amino acid sequence 100% identical with the amino acid sequence of SEQ ID NO: 26. In some embodiments, the antibody comprises a variable light chain (VL) region having an amino acid sequence 100% identical with the amino acid sequence of SEQ ID NO: 30. In some embodiments, the antibody comprises: (a) a complementarity determining region (CDR) H1 having an amino acid sequence 100% identical to the amino acid sequence of SEQ ID NO: 27, a CDR H2 having an amino acid sequence 100% identical to the amino acid sequence of SEQ ID NO: 28, and a CDR H3 having an amino acid sequence 100% identical to the amino acid sequence of SEQ ID NO: 29; and (b) a complementarity determining region (CDR) L1 having 100% sequence identity to an amino acid sequence of SEQ ID NO: 31, a CDR L2 having an amino acid sequence 100% identical to the amino acid sequence of ESS, and a CDR L3 having an amino acid sequence 100% identical to the amino acid sequence of SEQ ID NO: 32. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 26 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 30. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 26 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 30. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 26 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 30. In some embodiments, the antibody comprises a variable heavy chain (VH) region having an amino acid sequence with at least 95% (e.g., at least 95%, 96%, 97%, 98%, 99%, or more) identity to SEQ ID NO: 26 and a variable light chain (VL) region having an amino acid sequence with at least 95% (e.g., at least 95%, 96%, 97%, 98%, 99%, or more) identity to SEQ ID NO: 30. In some embodiments, the antibody comprises a variable heavy chain (VH) region having an amino acid sequence 100% identical with the amino acid sequence of SEQ ID NO: 26 and a variable light chain (VL) region having an amino acid sequence 100% identical with the amino acid sequence of SEQ ID NO: 30. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 26 and comprising therein a complementarity determining region (CDR) H1 having an amino acid sequence 100% identical to the amino acid sequence of SEQ ID NO: 27, a CDR H2 having an amino acid sequence 100% identical to the amino acid sequence of SEQ ID NO: 28, and a CDR H3 having an amino acid sequence 100% identical to the amino acid sequence of SEQ ID NO: 29. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 30 and comprising therein a complementarity determining region (CDR) L1 having 100% sequence identity to an amino acid sequence of SEQ ID NO: 31, a CDR L2 having an amino acid sequence 100% identical to the amino acid sequence of ESS, and a CDR L3 having an amino acid sequence 100% identical to the amino acid sequence of SEQ ID NO: 32. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 26 and comprising therein a complementarity determining region (CDR) H1 having an amino acid sequence 100% identical to the amino acid sequence of SEQ ID NO: 27, a CDR H2 having an amino acid sequence 100% identical to the amino acid sequence of SEQ ID NO: 28, and a CDR H3 having an amino acid sequence 100% identical to the amino acid sequence of SEQ ID NO: 29; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 30 and comprising therein a complementarity determining region (CDR) L1 having 100% sequence identity to an amino acid sequence of SEQ ID NO: 31, a CDR L2 having an amino acid sequence 100% identical to the amino acid sequence of ESS, and a CDR L3 having an amino acid sequence 100% identical to the amino acid sequence of SEQ ID NO: 32.

In some embodiments, the antibody comprises a complementarity determining region (CDR) H1 having an amino acid sequence 100% identical to the amino acid sequence of SEQ ID NO: 34, a CDR H2 having an amino acid sequence 100% identical to the amino acid sequence of SEQ ID NO: 35, and a CDR H3 having an amino acid sequence 100% identical to the amino acid sequence of SEQ ID NO: 36. In some embodiments, the antibody comprises a complementarity determining region (CDR) L1 having 100% sequence identity to an amino acid sequence of SEQ ID NO: 38, a CDR L2 having an amino acid sequence 100% identical to the amino acid sequence of DAT, and a CDR L3 having an amino acid sequence 100% identical to the amino acid sequence of SEQ ID NO: 39. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 33. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 37. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 33. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 37. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 33. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 37. In some embodiments, the antibody comprises a variable heavy chain (VH) region having an amino acid sequence with at least 95% (e.g., at least 95%, 96%, 97%, 98%, 99%, or more) identity to SEQ ID NO: 33. In some embodiments, the antibody comprises a variable light chain (VL) region having an amino acid sequence with at least 95% (e.g., at least 95%, 96%, 97%, 98%, 99%, or more) identity to SEQ ID NO: 37. In some embodiments, the antibody comprises a variable heavy chain (VH) region having an amino acid sequence 100% identical with the amino acid sequence of SEQ ID NO: 33. In some embodiments, the antibody comprises a variable light chain (VL) region having an amino acid sequence 100% identical with the amino acid sequence of SEQ ID NO: 37. In some embodiments, the antibody comprises: (a) a complementarity determining region (CDR) H1 having an amino acid sequence 100% identical to the amino acid sequence of SEQ ID NO: 34, a CDR H2 having an amino acid sequence 100% identical to the amino acid sequence of SEQ ID NO: 35, and a CDR H3 having an amino acid sequence 100% identical to the amino acid sequence of SEQ ID NO: 36; and (b) a complementarity determining region (CDR) L1 having 100% sequence identity to an amino acid sequence SEQ ID NO: 38, a CDR L2 having an amino acid sequence 100% identical to the amino acid sequence of DAT, and a CDR L3 having an amino acid sequence 100% identical to the amino acid sequence of SEQ ID NO: 39. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 33 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 37. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 33 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 37. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 33 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 37. In some embodiments, the antibody comprises a variable heavy chain (VH) region having an amino acid sequence with at least 95% (e.g., at least 95%, 96%, 97%, 98%, 99%, or more) identity to SEQ ID NO: 33 and a variable light chain (VL) region having an amino acid sequence with at least 95% (e.g., at least 95%, 96%, 97%, 98%, 99%, or more) identity to SEQ ID NO: 37. In some embodiments, the antibody comprises a variable heavy chain (VH) region having an amino acid sequence 100% identical with the amino acid sequence of SEQ ID NO: 33 and a variable light chain (VL) region having an amino acid sequence 100% identical with the amino acid sequence of SEQ ID NO: 37. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 33 and comprising therein a complementarity determining region (CDR) H1 having an amino acid sequence 100% identical to the amino acid sequence SEQ ID NO: 34, a CDR H2 having an amino acid sequence 100% identical to the amino acid sequence of SEQ ID NO: 35, and a CDR H3 having an amino acid sequence 100% identical to the amino acid sequence of SEQ ID NO: 36. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 37 and comprising therein a complementarity determining region (CDR) L1 having 100% sequence identity to an amino acid sequence of SEQ ID NO: 38, a CDR L2 having an amino acid sequence 100% identical to the amino acid sequence of DAT, and a CDR L3 having an amino acid sequence 100% identical to the amino acid sequence of SEQ ID NO: 39. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 33 and comprising therein a complementarity determining region (CDR) H1 having an amino acid sequence 100% identical to the amino acid sequence of SEQ ID NO: 34, a CDR H2 having an amino acid sequence 100% identical to the amino acid sequence of SEQ ID NO: 35, and a CDR H3 having an amino acid sequence 100% identical to the amino acid sequence of SEQ ID NO: 36; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 37 and comprising therein a complementarity determining region (CDR) L1 having 100% sequence identity to an amino acid sequence of SEQ ID NO: 38, a CDR L2 having an amino acid sequence 100% identical to the amino acid sequence of DAT, and a CDR L3 having an amino acid sequence 100% identical to the amino acid sequence of SEQ ID NO: 39. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 455. In some embodiments, the antibody comprises a second heavy chain region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 475. In some embodiments, the antibody comprises a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 465. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 455, a second heavy chain region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 475, and a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 465.

In some embodiments, the antibody comprises a complementarity determining region (CDR) H1 having an amino acid sequence 100% identical to the amino acid sequence of SEQ ID NO: 41, a CDR H2 having an amino acid sequence 100% identical to the amino acid sequence of SEQ ID NO: 42, and a CDR H3 having an amino acid sequence 100% identical to the amino acid sequence of SEQ ID NO: 43. In some embodiments, the antibody comprises a complementarity determining region (CDR) L1 having 100% sequence identity to an amino acid sequence of SEQ ID NO: 45, a CDR L2 having an amino acid sequence 100% identical to the amino acid sequence of GSS, and a CDR L3 having an amino acid sequence 100% identical to the amino acid sequence of SEQ ID NO: 46. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 40. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 44. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 40. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 44. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 40. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 44. In some embodiments, the antibody comprises a variable heavy chain (VH) region having an amino acid sequence with at least 95% (e.g., at least 95%, 96%, 97%, 98%, 99%, or more) identity to SEQ ID NO: 40. In some embodiments, the antibody comprises a variable light chain (VL) region having an amino acid sequence with at least 95% (e.g., at least 95%, 96%, 97%, 98%, 99%, or more) identity to SEQ ID NO: 44. In some embodiments, the antibody comprises a variable heavy chain (VH) region having an amino acid sequence 100% identical with the amino acid sequence of SEQ ID NO: 40. In some embodiments, the antibody comprises a variable light chain (VL) region having an amino acid sequence 100% identical with the amino acid sequence of SEQ ID NO: 44. In some embodiments, the antibody comprises: (a) a complementarity determining region (CDR) H1 having an amino acid sequence 100% identical to the amino acid sequence of SEQ ID NO: 41, a CDR H2 having an amino acid sequence 100% identical to the amino acid sequence of SEQ ID NO: 42, and a CDR H3 having an amino acid sequence 100% identical to the amino acid sequence of SEQ ID NO: 43; and (b) a complementarity determining region (CDR) L1 having 100% sequence identity to an amino acid sequence of SEQ ID NO: 45, a CDR L2 having an amino acid sequence 100% identical to the amino acid sequence of GSS, and a CDR L3 having an amino acid sequence 100% identical to the amino acid sequence of SEQ ID NO: 46. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 40 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 44. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 40 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 44. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 40 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 44. In some embodiments, the antibody comprises a variable heavy chain (VH) region having an amino acid sequence with at least 95% (e.g., at least 95%, 96%, 97%, 98%, 99%, or more) identity to SEQ ID NO: 40 and a variable light chain (VL) region having an amino acid sequence with at least 95% (e.g., at least 95%, 96%, 97%, 98%, 99%, or more) identity to SEQ ID NO: 44. In some embodiments, the antibody comprises a variable heavy chain (VH) region having an amino acid sequence 100% identical with the amino acid sequence of SEQ ID NO: 40 and a variable light chain (VL) region having an amino acid sequence 100% identical with the amino acid sequence of SEQ ID NO: 44. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 40 and comprising therein a complementarity determining region (CDR) H1 having an amino acid sequence 100% identical to the amino acid sequence of SEQ ID NO: 41, a CDR H2 having an amino acid sequence 100% identical to the amino acid sequence of SEQ ID NO: 42, and a CDR H3 having an amino acid sequence 100% identical to the amino acid sequence of SEQ ID NO: 43. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 44 and comprising therein a complementarity determining region (CDR) L1 having 100% sequence identity to an amino acid sequence of SEQ ID NO: 45, a CDR L2 having an amino acid sequence 100% identical to the amino acid sequence of GSS, and a CDR L3 having an amino acid sequence 100% identical to the amino acid sequence of SEQ ID NO: 46. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 40 and comprising therein a complementarity determining region (CDR) H1 having an amino acid sequence 100% identical to the amino acid sequence of SEQ ID NO: 41, a CDR H2 having an amino acid sequence 100% identical to the amino acid sequence of SEQ ID NO: 42, and a CDR H3 having an amino acid sequence 100% identical to the amino acid sequence of SEQ ID NO: 43; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 44 and comprising therein a complementarity determining region (CDR) L1 having 100% sequence identity to an amino acid sequence of SEQ ID NO: 45, a CDR L2 having an amino acid sequence 100% identical to the amino acid sequence of GSS, and a CDR L3 having an amino acid sequence 100% identical to the amino acid sequence of SEQ ID NO: 46. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 456. In some embodiments, the antibody comprises a second heavy chain region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 476. In some embodiments, the antibody comprises a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 466. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 456, a second heavy chain region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 476, and a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 466.

In some embodiments, the antibody comprises a complementarity determining region (CDR) H1 having an amino acid sequence 100% identical to the amino acid sequence of SEQ ID NO: 48, a CDR H2 having an amino acid sequence 100% identical to the amino acid sequence of SEQ ID NO: 49, and a CDR H3 having an amino acid sequence 100% identical to the amino acid sequence of SEQ ID NO: 50. In some embodiments, the antibody comprises a complementarity determining region (CDR) L1 having 100% sequence identity to an amino acid sequence of SEQ ID NO: 52, a CDR L2 having an amino acid sequence 100% identical to the amino acid sequence of DAT, and a CDR L3 having an amino acid sequence 100% identical to the amino acid sequence of SEQ ID NO: 53. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 47. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 51. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 47. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 51. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 47. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 51. In some embodiments, the antibody comprises a variable heavy chain (VH) region having an amino acid sequence with at least 95% (e.g., at least 95%, 96%, 97%, 98%, 99%, or more) identity to SEQ ID NO: 47. In some embodiments, the antibody comprises a variable light chain (VL) region having an amino acid sequence with at least 95% (e.g., at least 95%, 96%, 97%, 98%, 99%, or more) identity to SEQ ID NO: 51. In some embodiments, the antibody comprises a variable heavy chain (VH) region having an amino acid sequence 100% identical with the amino acid sequence of SEQ ID NO: 47. In some embodiments, the antibody comprises a variable light chain (VL) region having an amino acid sequence 100% identical with the amino acid sequence of SEQ ID NO: 51. In some embodiments, the antibody comprises: (a) a complementarity determining region (CDR) H1 having an amino acid sequence 100% identical to the amino acid sequence of SEQ ID NO: 48, a CDR H2 having an amino acid sequence 100% identical to the amino acid sequence of SEQ ID NO: 49, and a CDR H3 having an amino acid sequence 100% identical to the amino acid sequence of SEQ ID NO: 50; and (b) a complementarity determining region (CDR) L1 having 100% sequence identity to an amino acid sequence of SEQ ID NO: 52, a CDR L2 having an amino acid sequence 100% identical to the amino acid sequence of DAT, and a CDR L3 having an amino acid sequence 100% identical to the amino acid sequence of SEQ ID NO: 53. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 47 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 51. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 47 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 51. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 47 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 51. In some embodiments, the antibody comprises a variable heavy chain (VH) region having an amino acid sequence with at least 95% (e.g., at least 95%, 96%, 97%, 98%, 99%, or more) identity to SEQ ID NO: 47 and a variable light chain (VL) region having an amino acid sequence with at least 95% (e.g., at least 95%, 96%, 97%, 98%, 99%, or more) identity to SEQ ID NO: 51. In some embodiments, the antibody comprises a variable heavy chain (VH) region having an amino acid sequence 100% identical with the amino acid sequence of SEQ ID NO: 47 and a variable light chain (VL) region having an amino acid sequence 100% identical with the amino acid sequence of SEQ ID NO: 51. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 47 and comprising therein a complementarity determining region (CDR) H1 having an amino acid sequence 100% identical to the amino acid sequence of SEQ ID NO: 48, a CDR H2 having an amino acid sequence 100% identical to the amino acid sequence of SEQ ID NO: 49, and a CDR H3 having an amino acid sequence 100% identical to the amino acid sequence of SEQ ID NO: 50. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 51 and comprising therein a complementarity determining region (CDR) L1 having 100% sequence identity to an amino acid sequence of SEQ ID NO: 52, a CDR L2 having an amino acid sequence 100% identical to the amino acid sequence of DAT, and a CDR L3 having an amino acid sequence 100% identical to the amino acid sequence of SEQ ID NO: 53. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 47 and comprising therein a complementarity determining region (CDR) H1 having an amino acid sequence 100% identical to the amino acid sequence of SEQ ID NO: 48, a CDR H2 having an amino acid sequence 100% identical to the amino acid sequence of SEQ ID NO: 49, and a CDR H3 having an amino acid sequence 100% identical to the amino acid sequence of SEQ ID NO: 50; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 51 and comprising therein a complementarity determining region (CDR) L1 having 100% sequence identity to an amino acid sequence of SEQ ID NO: 52, a CDR L2 having an amino acid sequence 100% identical to the amino acid sequence of DAT, and a CDR L3 having an amino acid sequence 100% identical to the amino acid sequence of SEQ ID NO: 53.

In some embodiments, the antibody comprises a complementarity determining region (CDR) H1 having an amino acid sequence 100% identical to the amino acid sequence of SEQ ID NO: 55, a CDR H2 having an amino acid sequence 100% identical to the amino acid sequence of SEQ ID NO: 56, and a CDR H3 having an amino acid sequence 100% identical to the amino acid sequence of SEQ ID NO: 57. In some embodiments, the antibody comprises a complementarity determining region (CDR) L1 having 100% sequence identity to an amino acid sequence of SEQ ID NO: 59, a CDR L2 having an amino acid sequence 100% identical to the amino acid sequence of GAS, and a CDR L3 having an amino acid sequence 100% identical to the amino acid sequence of SEQ ID NO: 60. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 54. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 58. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 54. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 58. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 54. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 58. In some embodiments, the antibody comprises a variable heavy chain (VH) region having an amino acid sequence with at least 95% (e.g., at least 95%, 96%, 97%, 98%, 99%, or more) identity to SEQ ID NO: 54. In some embodiments, the antibody comprises a variable light chain (VL) region having an amino acid sequence with at least 95% (e.g., at least 95%, 96%, 97%, 98%, 99%, or more) identity to SEQ ID NO: 58. In some embodiments, the antibody comprises a variable heavy chain (VH) region having an amino acid sequence 100% identical with the amino acid sequence of SEQ ID NO: 54. In some embodiments, the antibody comprises a variable light chain (VL) region having an amino acid sequence 100% identical with the amino acid sequence of SEQ ID NO: 58. In some embodiments, the antibody comprises: (a) a complementarity determining region (CDR) H1 having an amino acid sequence 100% identical to the amino acid sequence of SEQ ID NO: 55, a CDR H2 having an amino acid sequence 100% identical to the amino acid sequence of SEQ ID NO: 56, and a CDR H3 having an amino acid sequence 100% identical to the amino acid sequence of SEQ ID NO: 57; and (b) a complementarity determining region (CDR) L1 having 100% sequence identity to an amino acid sequence of SEQ ID NO: 59, a CDR L2 having an amino acid sequence 100% identical to the amino acid sequence of GAS, and a CDR L3 having an amino acid sequence 100% identical to the amino acid sequence of SEQ ID NO: 60. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 54 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 58. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 54 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 58. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 54 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 58. In some embodiments, the antibody comprises a variable heavy chain (VH) region having an amino acid sequence with at least 95% (e.g., at least 95%, 96%, 97%, 98%, 99%, or more) identity to SEQ ID NO: 54 and a variable light chain (VL) region having an amino acid sequence with at least 95% (e.g., at least 95%, 96%, 97%, 98%, 99%, or more) identity to SEQ ID NO: 58. In some embodiments, the antibody comprises a variable heavy chain (VH) region having an amino acid sequence 100% identical with the amino acid sequence of SEQ ID NO: 54 and a variable light chain (VL) region having an amino acid sequence 100% identical with the amino acid sequence of SEQ ID NO: 58. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 54 and comprising therein a complementarity determining region (CDR) H1 having an amino acid sequence 100% identical to the amino acid sequence of SEQ ID NO: 55, a CDR H2 having an amino acid sequence 100% identical to the amino acid sequence of SEQ ID NO: 56, and a CDR H3 having an amino acid sequence 100% identical to the amino acid sequence of SEQ ID NO: 57. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 58 and comprising therein a complementarity determining region (CDR) L1 having 100% sequence identity to an amino acid sequence SEQ ID NO: 59, a CDR L2 having an amino acid sequence 100% identical to the amino acid sequence of GAS, and a CDR L3 having an amino acid sequence 100% identical to the amino acid sequence of SEQ ID NO: 60. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 54 and comprising therein a complementarity determining region (CDR) H1 having an amino acid sequence 100% identical to the amino acid sequence of SEQ ID NO: 55, a CDR H2 having an amino acid sequence 100% identical to the amino acid sequence of SEQ ID NO: 56, and a CDR H3 having an amino acid sequence 100% identical to the amino acid sequence of SEQ ID NO: 57; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 58 and comprising therein a complementarity determining region (CDR) L1 having 100% sequence identity to an amino acid sequence of SEQ ID NO: 59, a CDR L2 having an amino acid sequence 100% identical to the amino acid sequence of GAS, and a CDR L3 having an amino acid sequence 100% identical to the amino acid sequence of SEQ ID NO: 60. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 457. In some embodiments, the antibody comprises a second heavy chain region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 477. In some embodiments, the antibody comprises a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 467. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 457, a second heavy chain region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 477, and a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 467.

In some embodiments, the antibody comprises a complementarity determining region (CDR) H1 having an amino acid sequence 100% identical to the amino acid sequence of SEQ ID NO: 62, a CDR H2 having an amino acid sequence 100% identical to the amino acid sequence of SEQ ID NO: 63, and a CDR H3 having an amino acid sequence 100% identical to the amino acid sequence of SEQ ID NO: 64. In some embodiments, the antibody comprises a complementarity determining region (CDR) L1 having 100% sequence identity to an amino acid sequence of SEQ ID NO: 66, a CDR L2 having an amino acid sequence 100% identical to the amino acid sequence of DAS, and a CDR L3 having an amino acid sequence 100% identical to the amino acid sequence of SEQ ID NO: 67. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 61. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 65. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 61. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 65. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 61. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 65. In some embodiments, the antibody comprises a variable heavy chain (VH) region having an amino acid sequence with at least 95% (e.g., at least 95%, 96%, 97%, 98%, 99%, or more) identity to SEQ ID NO: 61. In some embodiments, the antibody comprises a variable light chain (VL) region having an amino acid sequence with at least 95% (e.g., at least 95%, 96%, 97%, 98%, 99%, or more) identity to SEQ ID NO: 65. In some embodiments, the antibody comprises a variable heavy chain (VH) region having an amino acid sequence 100% identical with the amino acid sequence of SEQ ID NO: 61. In some embodiments, the antibody comprises a variable light chain (VL) region having an amino acid sequence 100% identical with the amino acid sequence of SEQ ID NO: 65. In some embodiments, the antibody comprises: (a) a complementarity determining region (CDR) H1 having an amino acid sequence 100% identical to the amino acid sequence of SEQ ID NO: 62, a CDR H2 having an amino acid sequence 100% identical to the amino acid sequence of SEQ ID NO: 63, and a CDR H3 having an amino acid sequence 100% identical to the amino acid sequence of SEQ ID NO: 64; and (b) a complementarity determining region (CDR) L1 having 100% sequence identity to an amino acid sequence of SEQ ID NO: 66, a CDR L2 having an amino acid sequence 100% identical to the amino acid sequence of DAS, and a CDR L3 having an amino acid sequence 100% identical to the amino acid sequence of SEQ ID NO: 67. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 61 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 65. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 61 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 65. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 61 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 65. In some embodiments, the antibody comprises a variable heavy chain (VH) region having an amino acid sequence with at least 95% (e.g., at least 95%, 96%, 97%, 98%, 99%, or more) identity to SEQ ID NO: 61 and a variable light chain (VL) region having an amino acid sequence with at least 95% (e.g., at least 95%, 96%, 97%, 98%, 99%, or more) identity to SEQ ID NO: 65. In some embodiments, the antibody comprises a variable heavy chain (VH) region having an amino acid sequence 100% identical with the amino acid sequence of SEQ ID NO: 61 and a variable light chain (VL) region having an amino acid sequence 100% identical with the amino acid sequence of SEQ ID NO: 65. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 61 and comprising therein a complementarity determining region (CDR) H1 having an amino acid sequence 100% identical to the amino acid sequence of SEQ ID NO: 62, a CDR H2 having an amino acid sequence 100% identical to the amino acid sequence of SEQ ID NO: 63, and a CDR H3 having an amino acid sequence 100% identical to the amino acid sequence of SEQ ID NO: 64. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 65 and comprising therein a complementarity determining region (CDR) L1 having 100% sequence identity to an amino acid sequence of SEQ ID NO: 66, a CDR L2 having an amino acid sequence 100% identical to the amino acid sequence of DAS, and a CDR L3 having an amino acid sequence 100% identical to the amino acid sequence of SEQ ID NO: 67. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 61 and comprising therein a complementarity determining region (CDR) H1 having an amino acid sequence 100% identical to the amino acid sequence of SEQ ID NO: 62, a CDR H2 having an amino acid sequence 100% identical to the amino acid sequence of SEQ ID NO: 63, and a CDR H3 having an amino acid sequence 100% identical to the amino acid sequence of SEQ ID NO: 64; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 65 and comprising therein a complementarity determining region (CDR) L1 having 100% sequence identity to an amino acid sequence of SEQ ID NO: 66, a CDR L2 having an amino acid sequence 100% identical to the amino acid sequence of DAS, and a CDR L3 having an amino acid sequence 100% identical to the amino acid sequence of SEQ ID NO: 67.

In some embodiments, the antibody comprises a complementarity determining region (CDR) H1 having an amino acid sequence 100% identical to the amino acid sequence of SEQ ID NO: 69, a CDR H2 having an amino acid sequence 100% identical to the amino acid sequence of SEQ ID NO: 70, and a CDR H3 having an amino acid sequence 100% identical to the amino acid sequence of SEQ ID NO: 71. In some embodiments, the antibody comprises a complementarity determining region (CDR) L1 having 100% sequence identity to an amino acid sequence of SEQ ID NO: 73, a CDR L2 having an amino acid sequence 100% identical to the amino acid sequence of GAS, and a CDR L3 having an amino acid sequence 100% identical to the amino acid sequence of SEQ ID NO: 74. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 68. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 72. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 68. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 72. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 68. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 72. In some embodiments, the antibody comprises a variable heavy chain (VH) region having an amino acid sequence with at least 95% (e.g., at least 95%, 96%, 97%, 98%, 99%, or more) identity to SEQ ID NO: 68. In some embodiments, the antibody comprises a variable light chain (VL) region having an amino acid sequence with at least 95% (e.g., at least 95%, 96%, 97%, 98%, 99%, or more) identity to SEQ ID NO: 72. In some embodiments, the antibody comprises a variable heavy chain (VH) region having an amino acid sequence 100% identical with the amino acid sequence of SEQ ID NO: 68. In some embodiments, the antibody comprises a variable light chain (VL) region having an amino acid sequence 100% identical with the amino acid sequence of SEQ ID NO: 72. In some embodiments, the antibody comprises: (a) a complementarity determining region (CDR) H1 having an amino acid sequence 100% identical to the amino acid sequence of SEQ ID NO: 69, a CDR H2 having an amino acid sequence 100% identical to the amino acid sequence of SEQ ID NO: 70, and a CDR H3 having an amino acid sequence 100% identical to the amino acid sequence of SEQ ID NO: 71; and (b) a complementarity determining region (CDR) L1 having 100% sequence identity to an amino acid sequence of SEQ ID NO: 73, a CDR L2 having an amino acid sequence 100% identical to the amino acid sequence of GAS, and a CDR L3 having an amino acid sequence 100% identical to the amino acid sequence of SEQ ID NO: 74. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 68 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 72. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 68 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 72. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 68 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 72. In some embodiments, the antibody comprises a variable heavy chain (VH) region having an amino acid sequence with at least 95% (e.g., at least 95%, 96%, 97%, 98%, 99%, or more) identity to SEQ ID NO: 68 and a variable light chain (VL) region having an amino acid sequence with at least 95% (e.g., at least 95%, 96%, 97%, 98%, 99%, or more) identity to SEQ ID NO: 72. In some embodiments, the antibody comprises a variable heavy chain (VH) region having an amino acid sequence 100% identical with the amino acid sequence of SEQ ID NO: 68 and a variable light chain (VL) region having an amino acid sequence 100% identical with the amino acid sequence of SEQ ID NO: 72. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 68 and comprising therein a complementarity determining region (CDR) H1 having an amino acid sequence 100% identical to the amino acid sequence of SEQ ID NO: 69, a CDR H2 having an amino acid sequence 100% identical to the amino acid sequence of SEQ ID NO: 70, and a CDR H3 having an amino acid sequence 100% identical to the amino acid sequence of SEQ ID NO: 71. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 72 and comprising therein a complementarity determining region (CDR) L1 having 100% sequence identity to an amino acid sequence of SEQ ID NO: 73, a CDR L2 having an amino acid sequence 100% identical to the amino acid sequence of GAS, and a CDR L3 having an amino acid sequence 100% identical to the amino acid sequence of SEQ ID NO: 74. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 68 and comprising therein a complementarity determining region (CDR) H1 having an amino acid sequence 100% identical to the amino acid sequence of SEQ ID NO: 69, a CDR H2 having an amino acid sequence 100% identical to the amino acid sequence of SEQ ID NO: 70, and a CDR H3 having an amino acid sequence 100% identical to the amino acid sequence of SEQ ID NO: 71; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 72 and comprising therein a complementarity determining region (CDR) L1 having 100% sequence identity to an amino acid sequence SEQ ID NO: 73, a CDR L2 having an amino acid sequence 100% identical to the amino acid sequence of GAS, and a CDR L3 having an amino acid sequence 100% identical to the amino acid sequence of SEQ ID NO: 74.

In some embodiments, the antibody comprises a complementarity determining region (CDR) H1 having an amino acid sequence 100% identical to the amino acid sequence of SEQ ID NO: 76, a CDR H2 having an amino acid sequence 100% identical to the amino acid sequence of SEQ ID NO: 77, and a CDR H3 having an amino acid sequence 100% identical to the amino acid sequence of SEQ ID NO: 78. In some embodiments, the antibody comprises a complementarity determining region (CDR) L1 having 100% sequence identity to an amino acid sequence of SEQ ID NO: 80, a CDR L2 having an amino acid sequence 100% identical to the amino acid sequence of HAS, and a CDR L3 having an amino acid sequence 100% identical to the amino acid sequence of SEQ ID NO: 81. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 75. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 79. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 75. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 79. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 75. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 79. In some embodiments, the antibody comprises a variable heavy chain (VH) region having an amino acid sequence with at least 95% (e.g., at least 95%, 96%, 97%, 98%, 99%, or more) identity to SEQ ID NO: 75. In some embodiments, the antibody comprises a variable light chain (VL) region having an amino acid sequence with at least 95% (e.g., at least 95%, 96%, 97%, 98%, 99%, or more) identity to SEQ ID NO: 79. In some embodiments, the antibody comprises a variable heavy chain (VH) region having an amino acid sequence 100% identical with the amino acid sequence of SEQ ID NO: 75. In some embodiments, the antibody comprises a variable light chain (VL) region having an amino acid sequence 100% identical with the amino acid sequence of SEQ ID NO: 79. In some embodiments, the antibody comprises: (a) a complementarity determining region (CDR) H1 having an amino acid sequence 100% identical to the amino acid sequence of SEQ ID NO: 76, a CDR H2 having an amino acid sequence 100% identical to the amino acid sequence of SEQ ID NO: 77, and a CDR H3 having an amino acid sequence 100% identical to the amino acid sequence of SEQ ID NO: 78; and (b) a complementarity determining region (CDR) L1 having 100% sequence identity to an amino acid sequence of SEQ ID NO: 80, a CDR L2 having an amino acid sequence 100% identical to the amino acid sequence of HAS, and a CDR L3 having an amino acid sequence 100% identical to the amino acid sequence of SEQ ID NO: 81. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 75 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 79. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 75 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 79. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 75 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 79. In some embodiments, the antibody comprises a variable heavy chain (VH) region having an amino acid sequence with at least 95% (e.g., at least 95%, 96%, 97%, 98%, 99%, or more) identity to SEQ ID NO: 75 and a variable light chain (VL) region having an amino acid sequence with at least 95% (e.g., at least 95%, 96%, 97%, 98%, 99%, or more) identity to SEQ ID NO: 79. In some embodiments, the antibody comprises a variable heavy chain (VH) region having an amino acid sequence 100% identical with the amino acid sequence of SEQ ID NO: 75 and a variable light chain (VL) region having an amino acid sequence 100% identical with the amino acid sequence of SEQ ID NO: 79. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 75 and comprising therein a complementarity determining region (CDR) H1 having an amino acid sequence 100% identical to the amino acid sequence of SEQ ID NO: 76, a CDR H2 having an amino acid sequence 100% identical to the amino acid sequence of SEQ ID NO: 77, and a CDR H3 having an amino acid sequence 100% identical to the amino acid sequence of SEQ ID NO: 78. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 79 and comprising therein a complementarity determining region (CDR) L1 having 100% sequence identity to an amino acid sequence of SEQ ID NO: 80, a CDR L2 having an amino acid sequence 100% identical to the amino acid sequence of HAS, and a CDR L3 having an amino acid sequence 100% identical to the amino acid sequence of SEQ ID NO: 81. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 75 and comprising therein a complementarity determining region (CDR) H1 having an amino acid sequence 100% identical to the amino acid sequence of SEQ ID NO: 76, a CDR H2 having an amino acid sequence 100% identical to the amino acid sequence of SEQ ID NO: 77, and a CDR H3 having an amino acid sequence 100% identical to the amino acid sequence of SEQ ID NO: 78; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 79 and comprising therein a complementarity determining region (CDR) L1 having 100% sequence identity to an amino acid sequence of SEQ ID NO: 80, a CDR L2 having an amino acid sequence 100% identical to the amino acid sequence of HAS, and a CDR L3 having an amino acid sequence 100% identical to the amino acid sequence of SEQ ID NO: 81.

In some embodiments, the antibody comprises a complementarity determining region (CDR) H1 having an amino acid sequence 100% identical to the amino acid sequence of SEQ ID NO: 83, a CDR H2 having an amino acid sequence 100% identical to the amino acid sequence of SEQ ID NO: 84, and a CDR H3 having an amino acid sequence 100% identical to the amino acid sequence of SEQ ID NO: 85. In some embodiments, the antibody comprises a complementarity determining region (CDR) L1 having 100% sequence identity to an amino acid sequence of SEQ ID NO: 87, a CDR L2 having an amino acid sequence 100% identical to the amino acid sequence of DAT, and a CDR L3 having an amino acid sequence 100% identical to the amino acid sequence of SEQ ID NO: 88. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 82. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 86. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 82. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 86. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 82. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 86. In some embodiments, the antibody comprises a variable heavy chain (VH) region having an amino acid sequence with at least 95% (e.g., at least 95%, 96%, 97%, 98%, 99%, or more) identity to SEQ ID NO: 82. In some embodiments, the antibody comprises a variable light chain (VL) region having an amino acid sequence with at least 95% (e.g., at least 95%, 96%, 97%, 98%, 99%, or more) identity to SEQ ID NO: 86. In some embodiments, the antibody comprises a variable heavy chain (VH) region having an amino acid sequence 100% identical with the amino acid sequence of SEQ ID NO: 82. In some embodiments, the antibody comprises a variable light chain (VL) region having an amino acid sequence 100% identical with the amino acid sequence of SEQ ID NO: 86. In some embodiments, the antibody comprises: (a) a complementarity determining region (CDR) H1 having an amino acid sequence 100% identical to the amino acid sequence of SEQ ID NO: 83, a CDR H2 having an amino acid sequence 100% identical to the amino acid sequence of SEQ ID NO: 84, and a CDR H3 having an amino acid sequence 100% identical to the amino acid sequence of SEQ ID NO: 85; and (b) a complementarity determining region (CDR) L1 having 100% sequence identity to an amino acid sequence of SEQ ID NO: 87, a CDR L2 having an amino acid sequence 100% identical to the amino acid sequence of DAT, and a CDR L3 having an amino acid sequence 100% identical to the amino acid sequence of SEQ ID NO: 88. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 82 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 86. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 82 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 86. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 82 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 86. In some embodiments, the antibody comprises a variable heavy chain (VH) region having an amino acid sequence with at least 95% (e.g., at least 95%, 96%, 97%, 98%, 99%, or more) identity to SEQ ID NO: 82 and a variable light chain (VL) region having an amino acid sequence with at least 95% (e.g., at least 95%, 96%, 97%, 98%, 99%, or more) identity to SEQ ID NO: 86. In some embodiments, the antibody comprises a variable heavy chain (VH) region having an amino acid sequence 100% identical with the amino acid sequence of SEQ ID NO: 82 and a variable light chain (VL) region having an amino acid sequence 100% identical with the amino acid sequence of SEQ ID NO: 86. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 82 and comprising therein a complementarity determining region (CDR) H1 having an amino acid sequence 100% identical to the amino acid sequence of SEQ ID NO: 83, a CDR H2 having an amino acid sequence 100% identical to the amino acid sequence of SEQ ID NO: 84, and a CDR H3 having an amino acid sequence 100% identical to the amino acid sequence of SEQ ID NO: 85. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 86 and comprising therein a complementarity determining region (CDR) L1 having 100% sequence identity to an amino acid sequence of SEQ ID NO: 87, a CDR L2 having an amino acid sequence 100% identical to the amino acid sequence of DAT, and a CDR L3 having an amino acid sequence 100% identical to the amino acid sequence of SEQ ID NO: 88. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 82 and comprising therein a complementarity determining region (CDR) H1 having an amino acid sequence 100% identical to the amino acid sequence of SEQ ID NO: 83, a CDR H2 having an amino acid sequence 100% identical to the amino acid sequence of SEQ ID NO: 84, and a CDR H3 having an amino acid sequence 100% identical to the amino acid sequence of SEQ ID NO: 85; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 86 and comprising therein a complementarity determining region (CDR) L1 having 100% sequence identity to an amino acid sequence of SEQ ID NO: 87, a CDR L2 having an amino acid sequence 100% identical to the amino acid sequence of DAT, and a CDR L3 having an amino acid sequence 100% identical to the amino acid sequence of SEQ ID NO: 88.

In some embodiments, the antibody comprises a complementarity determining region (CDR) H1 having an amino acid sequence 100% identical to the amino acid sequence of SEQ ID NO: 90, a CDR H2 having an amino acid sequence 100% identical to the amino acid sequence of SEQ ID NO: 91, and a CDR H3 having an amino acid sequence 100% identical to the amino acid sequence of SEQ ID NO: 92. In some embodiments, the antibody comprises a complementarity determining region (CDR) L1 having 100% sequence identity to an amino acid sequence of SEQ ID NO: 94, a CDR L2 having an amino acid sequence 100% identical to the amino acid sequence of GAS, and a CDR L3 having an amino acid sequence 100% identical to the amino acid sequence of SEQ ID NO: 95. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 89. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 93. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 89. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 93. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 89. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 93. In some embodiments, the antibody comprises a variable heavy chain (VH) region having an amino acid sequence with at least 95% (e.g., at least 95%, 96%, 97%, 98%, 99%, or more) identity to SEQ ID NO: 89. In some embodiments, the antibody comprises a variable light chain (VL) region having an amino acid sequence with at least 95% (e.g., at least 95%, 96%, 97%, 98%, 99%, or more) identity to SEQ ID NO: 93. In some embodiments, the antibody comprises a variable heavy chain (VH) region having an amino acid sequence 100% identical with the amino acid sequence of SEQ ID NO: 89. In some embodiments, the antibody comprises a variable light chain (VL) region having an amino acid sequence 100% identical with the amino acid sequence of SEQ ID NO: 93. In some embodiments, the antibody comprises: (a) a complementarity determining region (CDR) H1 having an amino acid sequence 100% identical to the amino acid sequence of SEQ ID NO: 90, a CDR H2 having an amino acid sequence 100% identical to the amino acid sequence of SEQ ID NO: 91, and a CDR H3 having an amino acid sequence 100% identical to the amino acid sequence of SEQ ID NO: 92; and (b) a complementarity determining region (CDR) L1 having 100% sequence identity to an amino acid sequence of SEQ ID NO: 94, a CDR L2 having an amino acid sequence 100% identical to the amino acid sequence of GAS, and a CDR L3 having an amino acid sequence 100% identical to the amino acid sequence of SEQ ID NO: 95. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 89 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 93. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 89 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 93. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 89 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 93. In some embodiments, the antibody comprises a variable heavy chain (VH) region having an amino acid sequence with at least 95% (e.g., at least 95%, 96%, 97%, 98%, 99%, or more) identity to SEQ ID NO: 89 and a variable light chain (VL) region having an amino acid sequence with at least 95% (e.g., at least 95%, 96%, 97%, 98%, 99%, or more) identity to SEQ ID NO: 93. In some embodiments, the antibody comprises a variable heavy chain (VH) region having an amino acid sequence 100% identical with the amino acid sequence of SEQ ID NO: 89 and a variable light chain (VL) region having an amino acid sequence 100% identical with the amino acid sequence of SEQ ID NO: 93. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 89 and comprising therein a complementarity determining region (CDR) H1 having an amino acid sequence 100% identical to the amino acid sequence of SEQ ID NO: 90, a CDR H2 having an amino acid sequence 100% identical to the amino acid sequence of SEQ ID NO: 91, and a CDR H3 having an amino acid sequence 100% identical to the amino acid sequence of SEQ ID NO: 92. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 93 and comprising therein a complementarity determining region (CDR) L1 having 100% sequence identity to an amino acid sequence of SEQ ID NO: 94, a CDR L2 having an amino acid sequence 100% identical to the amino acid sequence of GAS, and a CDR L3 having an amino acid sequence 100% identical to the amino acid sequence of SEQ ID NO: 95. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 89 and comprising therein a complementarity determining region (CDR) H1 having an amino acid sequence 100% identical to the amino acid sequence of SEQ ID NO: 90, a CDR H2 having an amino acid sequence 100% identical to the amino acid sequence of SEQ ID NO: 91, and a CDR H3 having an amino acid sequence 100% identical to the amino acid sequence of SEQ ID NO: 92; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 93 and comprising therein a complementarity determining region (CDR) L1 having 100% sequence identity to an amino acid sequence of SEQ ID NO: 94, a CDR L2 having an amino acid sequence 100% identical to the amino acid sequence of GAS, and a CDR L3 having an amino acid sequence 100% identical to the amino acid sequence of SEQ ID NO: 95. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 458. In some embodiments, the antibody comprises a second heavy chain region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 478. In some embodiments, the antibody comprises a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 468. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 458, a second heavy chain region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 478, and a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 468.

In some embodiments, the antibody comprises a complementarity determining region (CDR) H1 having an amino acid sequence 100% identical to the amino acid sequence of SEQ ID NO: 97, a CDR H2 having an amino acid sequence 100% identical to the amino acid sequence of SEQ ID NO: 98, and a CDR H3 having an amino acid sequence 100% identical to the amino acid sequence of SEQ ID NO: 99. In some embodiments, the antibody comprises a complementarity determining region (CDR) L1 having 100% sequence identity to an amino acid sequence of SEQ ID NO: 101, a CDR L2 having an amino acid sequence 100% identical to the amino acid sequence of DAS, and a CDR L3 having an amino acid sequence 100% identical to the amino acid sequence of SEQ ID NO: 102. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 96. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 100. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 96. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 100. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 96. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 100. In some embodiments, the antibody comprises a variable heavy chain (VH) region having an amino acid sequence with at least 95% (e.g., at least 95%, 96%, 97%, 98%, 99%, or more) identity to SEQ ID NO: 96. In some embodiments, the antibody comprises a variable light chain (VL) region having an amino acid sequence with at least 95% (e.g., at least 95%, 96%, 97%, 98%, 99%, or more) identity to SEQ ID NO: 100. In some embodiments, the antibody comprises a variable heavy chain (VH) region having an amino acid sequence 100% identical with the amino acid sequence of SEQ ID NO: 96. In some embodiments, the antibody comprises a variable light chain (VL) region having an amino acid sequence 100% identical with the amino acid sequence of SEQ ID NO: 100. In some embodiments, the antibody comprises: (a) a complementarity determining region (CDR) H1 having an amino acid sequence 100% identical to the amino acid sequence of SEQ ID NO: 97, a CDR H2 having an amino acid sequence 100% identical to the amino acid sequence of SEQ ID NO: 98, and a CDR H3 having an amino acid sequence 100% identical to the amino acid sequence of SEQ ID NO: 99; and (b) a complementarity determining region (CDR) L1 having 100% sequence identity to an amino acid sequence of SEQ ID NO: 101, a CDR L2 having an amino acid sequence 100% identical to the amino acid sequence of DAS, and a CDR L3 having an amino acid sequence 100% identical to the amino acid sequence of SEQ ID NO: 102. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 96 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 100. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 96 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 100. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 96 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 100. In some embodiments, the antibody comprises a variable heavy chain (VH) region having an amino acid sequence with at least 95% (e.g., at least 95%, 96%, 97%, 98%, 99%, or more) identity to SEQ ID NO: 96 and a variable light chain (VL) region having an amino acid sequence with at least 95% (e.g., at least 95%, 96%, 97%, 98%, 99%, or more) identity to SEQ ID NO: 100. In some embodiments, the antibody comprises a variable heavy chain (VH) region having an amino acid sequence 100% identical with the amino acid sequence of SEQ ID NO: 96 and a variable light chain (VL) region having an amino acid sequence 100% identical with the amino acid sequence of SEQ ID NO: 100. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 96 and comprising therein a complementarity determining region (CDR) H1 having an amino acid sequence 100% identical to the amino acid sequence SEQ ID NO: 97, a CDR H2 having an amino acid sequence 100% identical to the amino acid sequence of SEQ ID NO: 98, and a CDR H3 having an amino acid sequence 100% identical to the amino acid sequence of SEQ ID NO: 99. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 100 and comprising therein a complementarity determining region (CDR) L1 having 100% sequence identity to an amino acid sequence of SEQ ID NO: 101, a CDR L2 having an amino acid sequence 100% identical to the amino acid sequence of DAS, and a CDR L3 having an amino acid sequence 100% identical to the amino acid sequence of SEQ ID NO: 102. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 96 and comprising therein a complementarity determining region (CDR) H1 having an amino acid sequence 100% identical to the amino acid sequence of SEQ ID NO: 97, a CDR H2 having an amino acid sequence 100% identical to the amino acid sequence of SEQ ID NO: 98, and a CDR H3 having an amino acid sequence 100% identical to the amino acid sequence of SEQ ID NO: 99; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 100 and comprising therein a complementarity determining region (CDR) L1 having 100% sequence identity to an amino acid sequence of SEQ ID NO: 101, a CDR L2 having an amino acid sequence 100% identical to the amino acid sequence of DAS, and a CDR L3 having an amino acid sequence 100% identical to the amino acid sequence of SEQ ID NO: 102.

In some embodiments, the antibody comprises a complementarity determining region (CDR) H1 having an amino acid sequence 100% identical to the amino acid sequence of SEQ ID NO: 104, a CDR H2 having an amino acid sequence 100% identical to the amino acid sequence of SEQ ID NO: 105, and a CDR H3 having an amino acid sequence 100% identical to the amino acid sequence of SEQ ID NO: 106. In some embodiments, the antibody comprises a complementarity determining region (CDR) L1 having 100% sequence identity to an amino acid sequence of SEQ ID NO: 108, a CDR L2 having an amino acid sequence 100% identical to the amino acid sequence of DIS, and a CDR L3 having an amino acid sequence 100% identical to the amino acid sequence of SEQ ID NO: 109. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 103. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 107. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 103. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 107. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 103. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 107. In some embodiments, the antibody comprises a variable heavy chain (VH) region having an amino acid sequence with at least 95% (e.g., at least 95%, 96%, 97%, 98%, 99%, or more) identity to SEQ ID NO: 103. In some embodiments, the antibody comprises a variable light chain (VL) region having an amino acid sequence with at least 95% (e.g., at least 95%, 96%, 97%, 98%, 99%, or more) identity to SEQ ID NO: 107. In some embodiments, the antibody comprises a variable heavy chain (VH) region having an amino acid sequence 100% identical with the amino acid sequence of SEQ ID NO: 103. In some embodiments, the antibody comprises a variable light chain (VL) region having an amino acid sequence 100% identical with the amino acid sequence of SEQ ID NO: 107. In some embodiments, the antibody comprises: (a) a complementarity determining region (CDR) H1 having an amino acid sequence 100% identical to the amino acid sequence of SEQ ID NO: 104, a CDR H2 having an amino acid sequence 100% identical to the amino acid sequence of SEQ ID NO: 105, and a CDR H3 having an amino acid sequence 100% identical to the amino acid sequence of SEQ ID NO: 106; and (b) a complementarity determining region (CDR) L1 having 100% sequence identity to an amino acid sequence of SEQ ID NO: 108, a CDR L2 having an amino acid sequence 100% identical to the amino acid sequence of DIS, and a CDR L3 having an amino acid sequence 100% identical to the amino acid sequence of SEQ ID NO: 109. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 103 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 107. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 103 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 107. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 103 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 107. In some embodiments, the antibody comprises a variable heavy chain (VH) region having an amino acid sequence with at least 95% (e.g., at least 95%, 96%, 97%, 98%, 99%, or more) identity to SEQ ID NO: 103 and a variable light chain (VL) region having an amino acid sequence with at least 95% (e.g., at least 95%, 96%, 97%, 98%, 99%, or more) identity to SEQ ID NO: 107. In some embodiments, the antibody comprises a variable heavy chain (VH) region having an amino acid sequence 100% identical with the amino acid sequence of SEQ ID NO: 103 and a variable light chain (VL) region having an amino acid sequence 100% identical with the amino acid sequence of SEQ ID NO: 107. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 103 and comprising therein a complementarity determining region (CDR) H1 having an amino acid sequence 100% identical to the amino acid sequence of SEQ ID NO: 104, a CDR H2 having an amino acid sequence 100% identical to the amino acid sequence of SEQ ID NO: 105, and a CDR H3 having an amino acid sequence 100% identical to the amino acid sequence of SEQ ID NO: 106. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 107 and comprising therein a complementarity determining region (CDR) L1 having 100% sequence identity to an amino acid sequence of SEQ ID NO: 108, a CDR L2 having an amino acid sequence 100% identical to the amino acid sequence of DIS, and a CDR L3 having an amino acid sequence 100% identical to the amino acid sequence of SEQ ID NO: 109. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 103 and comprising therein a complementarity determining region (CDR) H1 having an amino acid sequence 100% identical to the amino acid sequence of SEQ ID NO: 104, a CDR H2 having an amino acid sequence 100% identical to the amino acid sequence of SEQ ID NO: 105, and a CDR H3 having an amino acid sequence 100% identical to the amino acid sequence of SEQ ID NO: 106; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 107 and comprising therein a complementarity determining region (CDR) L1 having 100% sequence identity to an amino acid sequence of SEQ ID NO: 108, a CDR L2 having an amino acid sequence 100% identical to the amino acid sequence of DIS, and a CDR L3 having an amino acid sequence 100% identical to the amino acid sequence of SEQ ID NO: 109.

In some embodiments, the antibody comprises a complementarity determining region (CDR) H1 having an amino acid sequence 100% identical to the amino acid sequence of SEQ ID NO: 111, a CDR H2 having an amino acid sequence 100% identical to the amino acid sequence of SEQ ID NO: 112, and a CDR H3 having an amino acid sequence 100% identical to the amino acid sequence of SEQ ID NO: 113. In some embodiments, the antibody comprises a complementarity determining region (CDR) L1 having 100% sequence identity to an amino acid sequence of SEQ ID NO: 115, a CDR L2 having an amino acid sequence 100% identical to the amino acid sequence of DAS, and a CDR L3 having an amino acid sequence 100% identical to the amino acid sequence of SEQ ID NO: 116. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 110. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 114. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 110. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 114. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 110. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 114. In some embodiments, the antibody comprises a variable heavy chain (VH) region having an amino acid sequence with at least 95% (e.g., at least 95%, 96%, 97%, 98%, 99%, or more) identity to SEQ ID NO: 110. In some embodiments, the antibody comprises a variable light chain (VL) region having an amino acid sequence with at least 95% (e.g., at least 95%, 96%, 97%, 98%, 99%, or more) identity to SEQ ID NO: 114. In some embodiments, the antibody comprises a variable heavy chain (VH) region having an amino acid sequence 100% identical with the amino acid sequence of SEQ ID NO: 110. In some embodiments, the antibody comprises a variable light chain (VL) region having an amino acid sequence 100% identical with the amino acid sequence of SEQ ID NO: 114. In some embodiments, the antibody comprises: (a) a complementarity determining region (CDR) H1 having an amino acid sequence 100% identical to the amino acid sequence of SEQ ID NO: 111, a CDR H2 having an amino acid sequence 100% identical to the amino acid sequence of SEQ ID NO: 112, and a CDR H3 having an amino acid sequence 100% identical to the amino acid sequence of SEQ ID NO: 113; and (b) a complementarity determining region (CDR) L1 having 100% sequence identity to an amino acid sequence of SEQ ID NO: 115, a CDR L2 having an amino acid sequence 100% identical to the amino acid sequence of DAS, and a CDR L3 having an amino acid sequence 100% identical to the amino acid sequence of SEQ ID NO: 116. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 110 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 114. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 110 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 114. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 110 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 114. In some embodiments, the antibody comprises a variable heavy chain (VH) region having an amino acid sequence with at least 95% (e.g., at least 95%, 96%, 97%, 98%, 99%, or more) identity to SEQ ID NO: 110 and a variable light chain (VL) region having an amino acid sequence with at least 95% (e.g., at least 95%, 96%, 97%, 98%, 99%, or more) identity to SEQ ID NO: 114. In some embodiments, the antibody comprises a variable heavy chain (VH) region having an amino acid sequence 100% identical with the amino acid sequence of SEQ ID NO: 110 and a variable light chain (VL) region having an amino acid sequence 100% identical with the amino acid sequence of SEQ ID NO: 114. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 110 and comprising therein a complementarity determining region (CDR) H1 having an amino acid sequence 100% identical to the amino acid sequence of SEQ ID NO: 111, a CDR H2 having an amino acid sequence 100% identical to the amino acid sequence of SEQ ID NO: 112, and a CDR H3 having an amino acid sequence 100% identical to the amino acid sequence of SEQ ID NO: 113. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 114 and comprising therein a complementarity determining region (CDR) L1 having 100% sequence identity to an amino acid sequence of SEQ ID NO: 115, a CDR L2 having an amino acid sequence 100% identical to the amino acid sequence of DAS, and a CDR L3 having an amino acid sequence 100% identical to the amino acid sequence of SEQ ID NO: 116. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 110 and comprising therein a complementarity determining region (CDR) H1 having an amino acid sequence 100% identical to the amino acid sequence of SEQ ID NO: 111, a CDR H2 having an amino acid sequence 100% identical to the amino acid sequence of SEQ ID NO: 112, and a CDR H3 having an amino acid sequence 100% identical to the amino acid sequence of SEQ ID NO: 113; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 114 and comprising therein a complementarity determining region (CDR) L1 having 100% sequence identity to an amino acid sequence of SEQ ID NO: 115, a CDR L2 having an amino acid sequence 100% identical to the amino acid sequence of DAS, and a CDR L3 having an amino acid sequence 100% identical to the amino acid sequence of SEQ ID NO: 116. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 459. In some embodiments, the antibody comprises a second heavy chain region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 479. In some embodiments, the antibody comprises a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 469. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 459, a second heavy chain region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 479, and a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 469.

In some embodiments, the antibody comprises a complementarity determining region (CDR) H1 having an amino acid sequence 100% identical to the amino acid sequence of SEQ ID NO: 118, a CDR H2 having an amino acid sequence 100% identical to the amino acid sequence of SEQ ID NO: 119, and a CDR H3 having an amino acid sequence 100% identical to the amino acid sequence of SEQ ID NO: 120. In some embodiments, the antibody comprises a complementarity determining region (CDR) L1 having 100% sequence identity to an amino acid sequence of SEQ ID NO: 122, a CDR L2 having an amino acid sequence 100% identical to the amino acid sequence of DAS, and a CDR L3 having an amino acid sequence 100% identical to the amino acid sequence of SEQ ID NO: 123. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 117. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 121. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 117. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 121. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 117. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 121. In some embodiments, the antibody comprises a variable heavy chain (VH) region having an amino acid sequence with at least 95% (e.g., at least 95%, 96%, 97%, 98%, 99%, or more) identity to SEQ ID NO: 117. In some embodiments, the antibody comprises a variable light chain (VL) region having an amino acid sequence with at least 95% (e.g., at least 95%, 96%, 97%, 98%, 99%, or more) identity to SEQ ID NO: 121. In some embodiments, the antibody comprises a variable heavy chain (VH) region having an amino acid sequence 100% identical with the amino acid sequence of SEQ ID NO: 117. In some embodiments, the antibody comprises a variable light chain (VL) region having an amino acid sequence 100% identical with the amino acid sequence of SEQ ID NO: 121. In some embodiments, the antibody comprises: (a) a complementarity determining region (CDR) H1 having an amino acid sequence 100% identical to the amino acid sequence of SEQ ID NO: 118, a CDR H2 having an amino acid sequence 100% identical to the amino acid sequence of SEQ ID NO: 119, and a CDR H3 having an amino acid sequence 100% identical to the amino acid sequence of SEQ ID NO: 120; and (b) a complementarity determining region (CDR) L1 having 100% sequence identity to an amino acid sequence of SEQ ID NO: 122, a CDR L2 having an amino acid sequence 100% identical to the amino acid sequence of DAS, and a CDR L3 having an amino acid sequence 100% identical to the amino acid sequence of SEQ ID NO: 123. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 117 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 121. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 117 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 121. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 117 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 121. In some embodiments, the antibody comprises a variable heavy chain (VH) region having an amino acid sequence with at least 95% (e.g., at least 95%, 96%, 97%, 98%, 99%, or more) identity to SEQ ID NO: 117 and a variable light chain (VL) region having an amino acid sequence with at least 95% (e.g., at least 95%, 96%, 97%, 98%, 99%, or more) identity to SEQ ID NO: 121. In some embodiments, the antibody comprises a variable heavy chain (VH) region having an amino acid sequence 100% identical with the amino acid sequence of SEQ ID NO: 117 and a variable light chain (VL) region having an amino acid sequence 100% identical with the amino acid sequence of SEQ ID NO: 121. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 117 and comprising therein a complementarity determining region (CDR) H1 having an amino acid sequence 100% identical to the amino acid sequence of SEQ ID NO: 118, a CDR H2 having an amino acid sequence 100% identical to the amino acid sequence of SEQ ID NO: 119, and a CDR H3 having an amino acid sequence 100% identical to the amino acid sequence of SEQ ID NO: 120. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 121 and comprising therein a complementarity determining region (CDR) L1 having 100% sequence identity to an amino acid sequence of SEQ ID NO: 122, a CDR L2 having an amino acid sequence 100% identical to the amino acid sequence of DAS, and a CDR L3 having an amino acid sequence 100% identical to the amino acid sequence of SEQ ID NO: 123. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 117 and comprising therein a complementarity determining region (CDR) H1 having an amino acid sequence 100% identical to the amino acid sequence of SEQ ID NO: 118, a CDR H2 having an amino acid sequence 100% identical to the amino acid sequence of SEQ ID NO: 119, and a CDR H3 having an amino acid sequence 100% identical to the amino acid sequence of SEQ ID NO: 120; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 121 and comprising therein a complementarity determining region (CDR) L1 having 100% sequence identity to an amino acid sequence of SEQ ID NO: 122, a CDR L2 having an amino acid sequence 100% identical to the amino acid sequence of DAS, and a CDR L3 having an amino acid sequence 100% identical to the amino acid sequence of SEQ ID NO: 123.

In some embodiments, the antibody comprises a complementarity determining region (CDR) H1 having an amino acid sequence 100% identical to the amino acid sequence of SEQ ID NO: 125, a CDR H2 having an amino acid sequence 100% identical to the amino acid sequence of SEQ ID NO: 126, and a CDR H3 having an amino acid sequence 100% identical to the amino acid sequence of SEQ ID NO: 127. In some embodiments, the antibody comprises a complementarity determining region (CDR) L1 having 100% sequence identity to an amino acid sequence of SEQ ID NO: 129, a CDR L2 having an amino acid sequence 100% identical to the amino acid sequence of EAS, and a CDR L3 having an amino acid sequence 100% identical to the amino acid sequence of SEQ ID NO: 130. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 124. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 128. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 124. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 128. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 124. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 128. In some embodiments, the antibody comprises a variable heavy chain (VH) region having an amino acid sequence with at least 95% (e.g., at least 95%, 96%, 97%, 98%, 99%, or more) identity to SEQ ID NO: 124. In some embodiments, the antibody comprises a variable light chain (VL) region having an amino acid sequence with at least 95% (e.g., at least 95%, 96%, 97%, 98%, 99%, or more) identity to SEQ ID NO: 128. In some embodiments, the antibody comprises a variable heavy chain (VH) region having an amino acid sequence 100% identical with the amino acid sequence of SEQ ID NO: 124. In some embodiments, the antibody comprises a variable light chain (VL) region having an amino acid sequence 100% identical with the amino acid sequence of SEQ ID NO: 128. In some embodiments, the antibody comprises: (a) a complementarity determining region (CDR) H1 having an amino acid sequence 100% identical to the amino acid sequence of SEQ ID NO: 125, a CDR H2 having an amino acid sequence 100% identical to the amino acid sequence of SEQ ID NO: 126, and a CDR H3 having an amino acid sequence 100% identical to the amino acid sequence of SEQ ID NO: 127; and (b) a complementarity determining region (CDR) L1 having 100% sequence identity to an amino acid sequence of SEQ ID NO: 129, a CDR L2 having an amino acid sequence 100% identical to the amino acid sequence of EAS, and a CDR L3 having an amino acid sequence 100% identical to the amino acid sequence of SEQ ID NO: 130. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 124 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 128. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 124 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 128. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 124 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 128. In some embodiments, the antibody comprises a variable heavy chain (VH) region having an amino acid sequence with at least 95% (e.g., at least 95%, 96%, 97%, 98%, 99%, or more) identity to SEQ ID NO: 124 and a variable light chain (VL) region having an amino acid sequence with at least 95% (e.g., at least 95%, 96%, 97%, 98%, 99%, or more) identity to SEQ ID NO: 128. In some embodiments, the antibody comprises a variable heavy chain (VH) region having an amino acid sequence 100% identical with the amino acid sequence of SEQ ID NO: 124 and a variable light chain (VL) region having an amino acid sequence 100% identical with the amino acid sequence of SEQ ID NO: 128. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 124 and comprising therein a complementarity determining region (CDR) H1 having an amino acid sequence 100% identical to the amino acid sequence of SEQ ID NO: 125, a CDR H2 having an amino acid sequence 100% identical to the amino acid sequence of SEQ ID NO: 126, and a CDR H3 having an amino acid sequence 100% identical to the amino acid sequence of SEQ ID NO: 127. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 128 and comprising therein a complementarity determining region (CDR) L1 having 100% sequence identity to an amino acid sequence of SEQ ID NO: 129, a CDR L2 having an amino acid sequence 100% identical to the amino acid sequence of EAS, and a CDR L3 having an amino acid sequence 100% identical to the amino acid sequence of SEQ ID NO: 130. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 124 and comprising therein a complementarity determining region (CDR) H1 having an amino acid sequence 100% identical to the amino acid sequence of SEQ ID NO: 125, a CDR H2 having an amino acid sequence 100% identical to the amino acid sequence of SEQ ID NO: 126, and a CDR H3 having an amino acid sequence 100% identical to the amino acid sequence of SEQ ID NO: 127; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 128 and comprising therein a complementarity determining region (CDR) L1 having 100% sequence identity to an amino acid sequence of SEQ ID NO: 129, a CDR L2 having an amino acid sequence 100% identical to the amino acid sequence of EAS, and a CDR L3 having an amino acid sequence 100% identical to the amino acid sequence of SEQ ID NO: 130.

In some embodiments, the antibody comprises a complementarity determining region (CDR) H1 having an amino acid sequence 100% identical to the amino acid sequence of SEQ ID NO: 132, a CDR H2 having an amino acid sequence 100% identical to the amino acid sequence of SEQ ID NO: 133, and a CDR H3 having an amino acid sequence 100% identical to the amino acid sequence of SEQ ID NO: 134. In some embodiments, the antibody comprises a complementarity determining region (CDR) L1 having 100% sequence identity to an amino acid sequence of SEQ ID NO: 136, a CDR L2 having an amino acid sequence 100% identical to the amino acid sequence of DAS, and a CDR L3 having an amino acid sequence 100% identical to the amino acid sequence of SEQ ID NO: 137. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 131. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 135. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 131. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 135. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 131. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 135. In some embodiments, the antibody comprises a variable heavy chain (VH) region having an amino acid sequence with at least 95% (e.g., at least 95%, 96%, 97%, 98%, 99%, or more) identity to SEQ ID NO: 131. In some embodiments, the antibody comprises a variable light chain (VL) region having an amino acid sequence with at least 95% (e.g., at least 95%, 96%, 97%, 98%, 99%, or more) identity to SEQ ID NO: 135. In some embodiments, the antibody comprises a variable heavy chain (VH) region having an amino acid sequence 100% identical with the amino acid sequence of SEQ ID NO: 131. In some embodiments, the antibody comprises a variable light chain (VL) region having an amino acid sequence 100% identical with the amino acid sequence of SEQ ID NO: 135. In some embodiments, the antibody comprises: (a) a complementarity determining region (CDR) H1 having an amino acid sequence 100% identical to the amino acid sequence of SEQ ID NO: 132, a CDR H2 having an amino acid sequence 100% identical to the amino acid sequence of SEQ ID NO: 133, and a CDR H3 having an amino acid sequence 100% identical to the amino acid sequence of SEQ ID NO: 134; and (b) a complementarity determining region (CDR) L1 having 100% sequence identity to an amino acid sequence of SEQ ID NO: 136, a CDR L2 having an amino acid sequence 100% identical to the amino acid sequence of DAS, and a CDR L3 having an amino acid sequence 100% identical to the amino acid sequence of SEQ ID NO: 137. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 131 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 135. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 131 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 135. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 131 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 135. In some embodiments, the antibody comprises a variable heavy chain (VH) region having an amino acid sequence with at least 95% (e.g., at least 95%, 96%, 97%, 98%, 99%, or more) identity to SEQ ID NO: 131 and a variable light chain (VL) region having an amino acid sequence with at least 95% (e.g., at least 95%, 96%, 97%, 98%, 99%, or more) identity to SEQ ID NO: 135. In some embodiments, the antibody comprises a variable heavy chain (VH) region having an amino acid sequence 100% identical with the amino acid sequence of SEQ ID NO: 131 and a variable light chain (VL) region having an amino acid sequence 100% identical with the amino acid sequence of SEQ ID NO: 135. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 131 and comprising therein a complementarity determining region (CDR) H1 having an amino acid sequence 100% identical to the amino acid sequence of SEQ ID NO: 132, a CDR H2 having an amino acid sequence 100% identical to the amino acid sequence of SEQ ID NO: 133, and a CDR H3 having an amino acid sequence 100% identical to the amino acid sequence of SEQ ID NO: 134. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 135 and comprising therein a complementarity determining region (CDR) L1 having 100% sequence identity to an amino acid sequence of SEQ ID NO: 136, a CDR L2 having an amino acid sequence 100% identical to the amino acid sequence of DAS, and a CDR L3 having an amino acid sequence 100% identical to the amino acid sequence of SEQ ID NO: 137. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 131 and comprising therein a complementarity determining region (CDR) H1 having an amino acid sequence 100% identical to the amino acid sequence of SEQ ID NO: 132, a CDR H2 having an amino acid sequence 100% identical to the amino acid sequence of SEQ ID NO: 133, and a CDR H3 having an amino acid sequence 100% identical to the amino acid sequence of SEQ ID NO: 134; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 135 and comprising therein a complementarity determining region (CDR) L1 having 100% sequence identity to an amino acid sequence of SEQ ID NO: 136, a CDR L2 having an amino acid sequence 100% identical to the amino acid sequence of DAS, and a CDR L3 having an amino acid sequence 100% identical to the amino acid sequence of SEQ ID NO: 137.

In some embodiments, the antibody comprises a complementarity determining region (CDR) H1 having an amino acid sequence 100% identical to the amino acid sequence of SEQ ID NO: 139, a CDR H2 having an amino acid sequence 100% identical to the amino acid sequence of SEQ ID NO: 140, and a CDR H3 having an amino acid sequence 100% identical to the amino acid sequence of SEQ ID NO: 141. In some embodiments, the antibody comprises a complementarity determining region (CDR) L1 having 100% sequence identity to an amino acid sequence of SEQ ID NO: 143, a CDR L2 having an amino acid sequence 100% identical to the amino acid sequence of DAS, and a CDR L3 having an amino acid sequence 100% identical to the amino acid sequence of SEQ ID NO: 144. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 138. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 142. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 138. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 142. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 138. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 142. In some embodiments, the antibody comprises a variable heavy chain (VH) region having an amino acid sequence with at least 95% (e.g., at least 95%, 96%, 97%, 98%, 99%, or more) identity to SEQ ID NO: 138. In some embodiments, the antibody comprises a variable light chain (VL) region having an amino acid sequence with at least 95% (e.g., at least 95%, 96%, 97%, 98%, 99%, or more) identity to SEQ ID NO: 142. In some embodiments, the antibody comprises a variable heavy chain (VH) region having an amino acid sequence 100% identical with the amino acid sequence of SEQ ID NO: 138. In some embodiments, the antibody comprises a variable light chain (VL) region having an amino acid sequence 100% identical with the amino acid sequence of SEQ ID NO: 142. In some embodiments, the antibody comprises: (a) a complementarity determining region (CDR) H1 having an amino acid sequence 100% identical to the amino acid sequence of SEQ ID NO: 139, a CDR H2 having an amino acid sequence 100% identical to the amino acid sequence of SEQ ID NO: 140, and a CDR H3 having an amino acid sequence 100% identical to the amino acid sequence of SEQ ID NO: 141; and (b) a complementarity determining region (CDR) L1 having 100% sequence identity to an amino acid sequence of SEQ ID NO: 143, a CDR L2 having an amino acid sequence 100% identical to the amino acid sequence of DAS, and a CDR L3 having an amino acid sequence 100% identical to the amino acid sequence of SEQ ID NO: 144. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 138 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 142. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 138 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 142. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 138 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 142. In some embodiments, the antibody comprises a variable heavy chain (VH) region having an amino acid sequence with at least 95% (e.g., at least 95%, 96%, 97%, 98%, 99%, or more) identity to SEQ ID NO: 138 and a variable light chain (VL) region having an amino acid sequence with at least 95% (e.g., at least 95%, 96%, 97%, 98%, 99%, or more) identity to SEQ ID NO: 142. In some embodiments, the antibody comprises a variable heavy chain (VH) region having an amino acid sequence 100% identical with the amino acid sequence of SEQ ID NO: 138 and a variable light chain (VL) region having an amino acid sequence 100% identical with the amino acid sequence of SEQ ID NO: 142. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 138 and comprising therein a complementarity determining region (CDR) H1 having an amino acid sequence 100% identical to the amino acid sequence of SEQ ID NO: 139, a CDR H2 having an amino acid sequence 100% identical to the amino acid sequence of SEQ ID NO: 140, and a CDR H3 having an amino acid sequence 100% identical to the amino acid sequence of SEQ ID NO: 141. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 142 and comprising therein a complementarity determining region (CDR) L1 having 100% sequence identity to an amino acid sequence of SEQ ID NO: 143, a CDR L2 having an amino acid sequence 100% identical to the amino acid sequence of DAS, and a CDR L3 having an amino acid sequence 100% identical to the amino acid sequence of SEQ ID NO: 144. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 138 and comprising therein a complementarity determining region (CDR) H1 having an amino acid sequence 100% identical to the amino acid sequence of SEQ ID NO: 139, a CDR H2 having an amino acid sequence 100% identical to the amino acid sequence of SEQ ID NO: 140, and a CDR H3 having an amino acid sequence 100% identical to the amino acid sequence of SEQ ID NO: 141; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 142 and comprising therein a complementarity determining region (CDR) L1 having 100% sequence identity to an amino acid sequence of SEQ ID NO: 143, a CDR L2 having an amino acid sequence 100% identical to the amino acid sequence of DAS, and a CDR L3 having an amino acid sequence 100% identical to the amino acid sequence of SEQ ID NO: 144.

In some embodiments, the antibody comprises a complementarity determining region (CDR) H1 having an amino acid sequence 100% identical to the amino acid sequence of SEQ ID NO: 146, a CDR H2 having an amino acid sequence 100% identical to the amino acid sequence of SEQ ID NO: 147, and a CDR H3 having an amino acid sequence 100% identical to the amino acid sequence of SEQ ID NO: 148. In some embodiments, the antibody comprises a complementarity determining region (CDR) L1 having 100% sequence identity to an amino acid sequence of SEQ ID NO: 150, a CDR L2 having an amino acid sequence 100% identical to the amino acid sequence of DAS, and a CDR L3 having an amino acid sequence 100% identical to the amino acid sequence of SEQ ID NO: 151. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 145. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 149. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 145. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 149. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 145. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 149. In some embodiments, the antibody comprises a variable heavy chain (VH) region having an amino acid sequence with at least 95% (e.g., at least 95%, 96%, 97%, 98%, 99%, or more) identity to SEQ ID NO: 145. In some embodiments, the antibody comprises a variable light chain (VL) region having an amino acid sequence with at least 95% (e.g., at least 95%, 96%, 97%, 98%, 99%, or more) identity to SEQ ID NO: 149. In some embodiments, the antibody comprises a variable heavy chain (VH) region having an amino acid sequence 100% identical with the amino acid sequence of SEQ ID NO: 145. In some embodiments, the antibody comprises a variable light chain (VL) region having an amino acid sequence 100% identical with the amino acid sequence of SEQ ID NO: 149. In some embodiments, the antibody comprises: (a) a complementarity determining region (CDR) H1 having an amino acid sequence 100% identical to the amino acid sequence of SEQ ID NO: 146, a CDR H2 having an amino acid sequence 100% identical to the amino acid sequence of SEQ ID NO: 147, and a CDR H3 having an amino acid sequence 100% identical to the amino acid sequence of SEQ ID NO: 148; and (b) a complementarity determining region (CDR) L1 having 100% sequence identity to an amino acid sequence of SEQ ID NO: 150, a CDR L2 having an amino acid sequence 100% identical to the amino acid sequence of DAS, and a CDR L3 having an amino acid sequence 100% identical to the amino acid sequence of SEQ ID NO: 151. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 145 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 149. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 145 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 149. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 145 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 149. In some embodiments, the antibody comprises a variable heavy chain (VH) region having an amino acid sequence with at least 95% (e.g., at least 95%, 96%, 97%, 98%, 99%, or more) identity to SEQ ID NO: 145 and a variable light chain (VL) region having an amino acid sequence with at least 95% (e.g., at least 95%, 96%, 97%, 98%, 99%, or more) identity to SEQ ID NO: 149. In some embodiments, the antibody comprises a variable heavy chain (VH) region having an amino acid sequence 100% identical with the amino acid sequence of SEQ ID NO: 145 and a variable light chain (VL) region having an amino acid sequence 100% identical with the amino acid sequence of SEQ ID NO: 149. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 145 and comprising therein a complementarity determining region (CDR) H1 having an amino acid sequence 100% identical to the amino acid sequence of SEQ ID NO: 146, a CDR H2 having an amino acid sequence 100% identical to the amino acid sequence of SEQ ID NO: 147, and a CDR H3 having an amino acid sequence 100% identical to the amino acid sequence of SEQ ID NO: 148. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 149 and comprising therein a complementarity determining region (CDR) L1 having 100% sequence identity to an amino acid sequence of SEQ ID NO: 150, a CDR L2 having an amino acid sequence 100% identical to the amino acid sequence of DAS, and a CDR L3 having an amino acid sequence 100% identical to the amino acid sequence of SEQ ID NO: 151. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 145 and comprising therein a complementarity determining region (CDR) H1 having an amino acid sequence 100% identical to the amino acid sequence of SEQ ID NO: 146, a CDR H2 having an amino acid sequence 100% identical to the amino acid sequence of SEQ ID NO: 147, and a CDR H3 having an amino acid sequence 100% identical to the amino acid sequence of SEQ ID NO: 148; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 149 and comprising therein a complementarity determining region (CDR) L1 having 100% sequence identity to an amino acid sequence of SEQ ID NO: 150, a CDR L2 having an amino acid sequence 100% identical to the amino acid sequence of DAS, and a CDR L3 having an amino acid sequence 100% identical to the amino acid sequence of SEQ ID NO: 151.

In some embodiments, the antibody comprises a complementarity determining region (CDR) H1 having an amino acid sequence 100% identical to the amino acid sequence of SEQ ID NO: 153, a CDR H2 having an amino acid sequence 100% identical to the amino acid sequence of SEQ ID NO: 154, and a CDR H3 having an amino acid sequence 100% identical to the amino acid sequence of SEQ ID NO: 155. In some embodiments, the antibody comprises a complementarity determining region (CDR) L1 having 100% sequence identity to an amino acid sequence of SEQ ID NO: 157, a CDR L2 having an amino acid sequence 100% identical to the amino acid sequence of DAS, and a CDR L3 having an amino acid sequence 100% identical to the amino acid sequence of SEQ ID NO: 158. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 152. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 156. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 152. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 156. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 152. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 156. In some embodiments, the antibody comprises a variable heavy chain (VH) region having an amino acid sequence with at least 95% (e.g., at least 95%, 96%, 97%, 98%, 99%, or more) identity to SEQ ID NO: 152. In some embodiments, the antibody comprises a variable light chain (VL) region having an amino acid sequence with at least 95% (e.g., at least 95%, 96%, 97%, 98%, 99%, or more) identity to SEQ ID NO: 156. In some embodiments, the antibody comprises a variable heavy chain (VH) region having an amino acid sequence 100% identical with the amino acid sequence of SEQ ID NO: 152. In some embodiments, the antibody comprises a variable light chain (VL) region having an amino acid sequence 100% identical with the amino acid sequence of SEQ ID NO: 156. In some embodiments, the antibody comprises: (a) a complementarity determining region (CDR) H1 having an amino acid sequence 100% identical to the amino acid sequence of SEQ ID NO: 153, a CDR H2 having an amino acid sequence 100% identical to the amino acid sequence of SEQ ID NO: 154, and a CDR H3 having an amino acid sequence 100% identical to the amino acid sequence of SEQ ID NO: 155; and (b) a complementarity determining region (CDR) L1 having 100% sequence identity to an amino acid sequence of SEQ ID NO: 157, a CDR L2 having an amino acid sequence 100% identical to the amino acid sequence of DAS, and a CDR L3 having an amino acid sequence 100% identical to the amino acid sequence of SEQ ID NO: 158. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 152 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 156. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 152 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 156. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 152 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 156. In some embodiments, the antibody comprises a variable heavy chain (VH) region having an amino acid sequence with at least 95% (e.g., at least 95%, 96%, 97%, 98%, 99%, or more) identity to SEQ ID NO: 152 and a variable light chain (VL) region having an amino acid sequence with at least 95% (e.g., at least 95%, 96%, 97%, 98%, 99%, or more) identity to SEQ ID NO: 156. In some embodiments, the antibody comprises a variable heavy chain (VH) region having an amino acid sequence 100% identical with the amino acid sequence of SEQ ID NO: 152 and a variable light chain (VL) region having an amino acid sequence 100% identical with the amino acid sequence of SEQ ID NO: 156. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 152 and comprising therein a complementarity determining region (CDR) H1 having an amino acid sequence 100% identical to the amino acid sequence of SEQ ID NO: 153, a CDR H2 having an amino acid sequence 100% identical to the amino acid sequence of SEQ ID NO: 154, and a CDR H3 having an amino acid sequence 100% identical to the amino acid sequence of SEQ ID NO: 155. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 156 and comprising therein a complementarity determining region (CDR) L1 having 100% sequence identity to an amino acid sequence of SEQ ID NO: 157, a CDR L2 having an amino acid sequence 100% identical to the amino acid sequence of DAS, and a CDR L3 having an amino acid sequence 100% identical to the amino acid sequence of SEQ ID NO: 158. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 152 and comprising therein a complementarity determining region (CDR) H1 having an amino acid sequence 100% identical to the amino acid sequence of SEQ ID NO: 153, a CDR H2 having an amino acid sequence 100% identical to the amino acid sequence of SEQ ID NO: 154, and a CDR H3 having an amino acid sequence 100% identical to the amino acid sequence of SEQ ID NO: 155; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 156 and comprising therein a complementarity determining region (CDR) L1 having 100% sequence identity to an amino acid sequence of SEQ ID NO: 157, a CDR L2 having an amino acid sequence 100% identical to the amino acid sequence of DAS, and a CDR L3 having an amino acid sequence 100% identical to the amino acid sequence of SEQ ID NO: 158.

In some embodiments, the antibody comprises a complementarity determining region (CDR) H1 having an amino acid sequence 100% identical to the amino acid sequence of SEQ ID NO: 160, a CDR H2 having an amino acid sequence 100% identical to the amino acid sequence of SEQ ID NO: 161, and a CDR H3 having an amino acid sequence 100% identical to the amino acid sequence of SEQ ID NO: 162. In some embodiments, the antibody comprises a complementarity determining region (CDR) L1 having 100% sequence identity to an amino acid sequence of SEQ ID NO: 164, a CDR L2 having an amino acid sequence 100% identical to the amino acid sequence of DAS, and a CDR L3 having an amino acid sequence 100% identical to the amino acid sequence of SEQ ID NO: 165. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 159. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 163. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 159. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 163. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 159. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 163. In some embodiments, the antibody comprises a variable heavy chain (VH) region having an amino acid sequence with at least 95% (e.g., at least 95%, 96%, 97%, 98%, 99%, or more) identity to SEQ ID NO: 159. In some embodiments, the antibody comprises a variable light chain (VL) region having an amino acid sequence with at least 95% (e.g., at least 95%, 96%, 97%, 98%, 99%, or more) identity to SEQ ID NO: 163. In some embodiments, the antibody comprises a variable heavy chain (VH) region having an amino acid sequence 100% identical with the amino acid sequence of SEQ ID NO: 159. In some embodiments, the antibody comprises a variable light chain (VL) region having an amino acid sequence 100% identical with the amino acid sequence of SEQ ID NO: 163. In some embodiments, the antibody comprises: (a) a complementarity determining region (CDR) H1 having an amino acid sequence 100% identical to the amino acid sequence of SEQ ID NO: 160, a CDR H2 having an amino acid sequence 100% identical to the amino acid sequence of SEQ ID NO: 161, and a CDR H3 having an amino acid sequence 100% identical to the amino acid sequence of SEQ ID NO: 162; and (b) a complementarity determining region (CDR) L1 having 100% sequence identity to an amino acid sequence of SEQ ID NO: 164, a CDR L2 having an amino acid sequence 100% identical to the amino acid sequence of DAS, and a CDR L3 having an amino acid sequence 100% identical to the amino acid sequence of SEQ ID NO: 165. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 159 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 163. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 159 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 163. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 159 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 163. In some embodiments, the antibody comprises a variable heavy chain (VH) region having an amino acid sequence with at least 95% (e.g., at least 95%, 96%, 97%, 98%, 99%, or more) identity to SEQ ID NO: 159 and a variable light chain (VL) region having an amino acid sequence with at least 95% (e.g., at least 95%, 96%, 97%, 98%, 99%, or more) identity to SEQ ID NO: 163. In some embodiments, the antibody comprises a variable heavy chain (VH) region having an amino acid sequence 100% identical with the amino acid sequence of SEQ ID NO: 159 and a variable light chain (VL) region having an amino acid sequence 100% identical with the amino acid sequence of SEQ ID NO: 163. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 159 and comprising therein a complementarity determining region (CDR) H1 having an amino acid sequence 100% identical to the amino acid sequence of SEQ ID NO: 160, a CDR H2 having an amino acid sequence 100% identical to the amino acid sequence of SEQ ID NO: 161, and a CDR H3 having an amino acid sequence 100% identical to the amino acid sequence of SEQ ID NO: 162. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 163 and comprising therein a complementarity determining region (CDR) L1 having 100% sequence identity to an amino acid sequence of SEQ ID NO: 164, a CDR L2 having an amino acid sequence 100% identical to the amino acid sequence of DAS, and a CDR L3 having an amino acid sequence 100% identical to the amino acid sequence of SEQ ID NO: 165. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 159 and comprising therein a complementarity determining region (CDR) H1 having an amino acid sequence 100% identical to the amino acid sequence of SEQ ID NO: 160, a CDR H2 having an amino acid sequence 100% identical to the amino acid sequence of SEQ ID NO: 161, and a CDR H3 having an amino acid sequence 100% identical to the amino acid sequence of SEQ ID NO: 162; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 163 and comprising therein a complementarity determining region (CDR) L1 having 100% sequence identity to an amino acid sequence of SEQ ID NO: 164, a CDR L2 having an amino acid sequence 100% identical to the amino acid sequence of DAS, and a CDR L3 having an amino acid sequence 100% identical to the amino acid sequence of SEQ ID NO: 165.

In some embodiments, the antibody comprises a complementarity determining region (CDR) H1 having an amino acid sequence 100% identical to the amino acid sequence of SEQ ID NO: 167, a CDR H2 having an amino acid sequence 100% identical to the amino acid sequence of SEQ ID NO: 168, and a CDR H3 having an amino acid sequence 100% identical to the amino acid sequence of SEQ ID NO: 169. In some embodiments, the antibody comprises a complementarity determining region (CDR) L1 having 100% sequence identity to an amino acid sequence of SEQ ID NO: 171, a CDR L2 having an amino acid sequence 100% identical to the amino acid sequence of GAS, and a CDR L3 having an amino acid sequence 100% identical to the amino acid sequence of SEQ ID NO: 172. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 166. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 170. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 166. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 170. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 166. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 170. In some embodiments, the antibody comprises a variable heavy chain (VH) region having an amino acid sequence with at least 95% (e.g., at least 95%, 96%, 97%, 98%, 99%, or more) identity to SEQ ID NO: 166. In some embodiments, the antibody comprises a variable light chain (VL) region having an amino acid sequence with at least 95% (e.g., at least 95%, 96%, 97%, 98%, 99%, or more) identity to SEQ ID NO: 170. In some embodiments, the antibody comprises a variable heavy chain (VH) region having an amino acid sequence 100% identical with the amino acid sequence of SEQ ID NO: 166. In some embodiments, the antibody comprises a variable light chain (VL) region having an amino acid sequence 100% identical with the amino acid sequence of SEQ ID NO: 170. In some embodiments, the antibody comprises: (a) a complementarity determining region (CDR) H1 having an amino acid sequence 100% identical to the amino acid sequence of SEQ ID NO: 167, a CDR H2 having an amino acid sequence 100% identical to the amino acid sequence of SEQ ID NO: 168, and a CDR H3 having an amino acid sequence 100% identical to the amino acid sequence of SEQ ID NO: 169; and (b) a complementarity determining region (CDR) L1 having 100% sequence identity to an amino acid sequence of SEQ ID NO: 171, a CDR L2 having an amino acid sequence 100% identical to the amino acid sequence of GAS, and a CDR L3 having an amino acid sequence 100% identical to the amino acid sequence of SEQ ID NO: 172. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 166 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 170. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 166 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 170. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 166 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 170. In some embodiments, the antibody comprises a variable heavy chain (VH) region having an amino acid sequence with at least 95% (e.g., at least 95%, 96%, 97%, 98%, 99%, or more) identity to SEQ ID NO: 166 and a variable light chain (VL) region having an amino acid sequence with at least 95% (e.g., at least 95%, 96%, 97%, 98%, 99%, or more) identity to SEQ ID NO: 170. In some embodiments, the antibody comprises a variable heavy chain (VH) region having an amino acid sequence 100% identical with the amino acid sequence of SEQ ID NO: 166 and a variable light chain (VL) region having an amino acid sequence 100% identical with the amino acid sequence of SEQ ID NO: 170. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 166 and comprising therein a complementarity determining region (CDR) H1 having an amino acid sequence 100% identical to the amino acid sequence of SEQ ID NO: 167, a CDR H2 having an amino acid sequence 100% identical to the amino acid sequence of SEQ ID NO: 168, and a CDR H3 having an amino acid sequence 100% identical to the amino acid sequence of SEQ ID NO: 169. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 170 and comprising therein a complementarity determining region (CDR) L1 having 100% sequence identity to an amino acid sequence of SEQ ID NO: 171, a CDR L2 having an amino acid sequence 100% identical to the amino acid sequence of GAS, and a CDR L3 having an amino acid sequence 100% identical to the amino acid sequence of SEQ ID NO: 172. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 166 and comprising therein a complementarity determining region (CDR) H1 having an amino acid sequence 100% identical to the amino acid sequence of SEQ ID NO: 167, a CDR H2 having an amino acid sequence 100% identical to the amino acid sequence of SEQ ID NO: 168, and a CDR H3 having an amino acid sequence 100% identical to the amino acid sequence of SEQ ID NO: 169; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 170 and comprising therein a complementarity determining region (CDR) L1 having 100% sequence identity to an amino acid sequence of SEQ ID NO: 171, a CDR L2 having an amino acid sequence 100% identical to the amino acid sequence of GAS, and a CDR L3 having an amino acid sequence 100% identical to the amino acid sequence of SEQ ID NO: 172.

In some embodiments, the antibody comprises a complementarity determining region (CDR) H1 having an amino acid sequence 100% identical to the amino acid sequence of SEQ ID NO: 174, a CDR H2 having an amino acid sequence 100% identical to the amino acid sequence of SEQ ID NO: 175, and a CDR H3 having an amino acid sequence 100% identical to the amino acid sequence of SEQ ID NO: 176. In some embodiments, the antibody comprises a complementarity determining region (CDR) L1 having 100% sequence identity to an amino acid sequence of SEQ ID NO: 178, a CDR L2 having an amino acid sequence 100% identical to the amino acid sequence of GAS, and a CDR L3 having an amino acid sequence 100% identical to the amino acid sequence of SEQ ID NO: 179. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 173. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 177. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 173. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 177. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 173. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 177. In some embodiments, the antibody comprises a variable heavy chain (VH) region having an amino acid sequence with at least 95% (e.g., at least 95%, 96%, 97%, 98%, 99%, or more) identity to SEQ ID NO: 173. In some embodiments, the antibody comprises a variable light chain (VL) region having an amino acid sequence with at least 95% (e.g., at least 95%, 96%, 97%, 98%, 99%, or more) identity to SEQ ID NO: 177. In some embodiments, the antibody comprises a variable heavy chain (VH) region having an amino acid sequence 100% identical with the amino acid sequence of SEQ ID NO: 173. In some embodiments, the antibody comprises a variable light chain (VL) region having an amino acid sequence 100% identical with the amino acid sequence of SEQ ID NO: 177. In some embodiments, the antibody comprises: (a) a complementarity determining region (CDR) H1 having an amino acid sequence 100% identical to the amino acid sequence of SEQ ID NO: 174, a CDR H2 having an amino acid sequence 100% identical to the amino acid sequence of SEQ ID NO: 175, and a CDR H3 having an amino acid sequence 100% identical to the amino acid sequence of SEQ ID NO: 176; and (b) a complementarity determining region (CDR) L1 having 100% sequence identity to an amino acid sequence of SEQ ID NO: 178, a CDR L2 having an amino acid sequence 100% identical to the amino acid sequence of GAS, and a CDR L3 having an amino acid sequence 100% identical to the amino acid sequence of SEQ ID NO: 179. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 173 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 177. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 173 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 177. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 173 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 177. In some embodiments, the antibody comprises a variable heavy chain (VH) region having an amino acid sequence with at least 95% (e.g., at least 95%, 96%, 97%, 98%, 99%, or more) identity to SEQ ID NO: 173 and a variable light chain (VL) region having an amino acid sequence with at least 95% (e.g., at least 95%, 96%, 97%, 98%, 99%, or more) identity to SEQ ID NO: 177. In some embodiments, the antibody comprises a variable heavy chain (VH) region having an amino acid sequence 100% identical with the amino acid sequence of SEQ ID NO: 173 and a variable light chain (VL) region having an amino acid sequence 100% identical with the amino acid sequence of SEQ ID NO: 177. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 173 and comprising therein a complementarity determining region (CDR) H1 having an amino acid sequence 100% identical to the amino acid sequence of SEQ ID NO: 174, a CDR H2 having an amino acid sequence 100% identical to the amino acid sequence of SEQ ID NO: 175, and a CDR H3 having an amino acid sequence 100% identical to the amino acid sequence of SEQ ID NO: 176. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 177 and comprising therein a complementarity determining region (CDR) L1 having 100% sequence identity to an amino acid sequence of SEQ ID NO: 178, a CDR L2 having an amino acid sequence 100% identical to the amino acid sequence of GAS, and a CDR L3 having an amino acid sequence 100% identical to the amino acid sequence of SEQ ID NO: 179. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 173 and comprising therein a complementarity determining region (CDR) H1 having an amino acid sequence 100% identical to the amino acid sequence of SEQ ID NO: 174, a CDR H2 having an amino acid sequence 100% identical to the amino acid sequence of SEQ ID NO: 175, and a CDR H3 having an amino acid sequence 100% identical to the amino acid sequence of SEQ ID NO: 176; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 177 and comprising therein a complementarity determining region (CDR) L1 having 100% sequence identity to an amino acid sequence of SEQ ID NO: 178, a CDR L2 having an amino acid sequence 100% identical to the amino acid sequence of GAS, and a CDR L3 having an amino acid sequence 100% identical to the amino acid sequence of SEQ ID NO: 179.

In some embodiments, the antibody comprises a complementarity determining region (CDR) H1 having an amino acid sequence 100% identical to the amino acid sequence of SEQ ID NO: 181, a CDR H2 having an amino acid sequence 100% identical to the amino acid sequence of SEQ ID NO: 182, and a CDR H3 having an amino acid sequence 100% identical to the amino acid sequence of SEQ ID NO: 183. In some embodiments, the antibody comprises a complementarity determining region (CDR) L1 having 100% sequence identity to an amino acid sequence of SEQ ID NO: 185, a CDR L2 having an amino acid sequence 100% identical to the amino acid sequence of DVS, and a CDR L3 having an amino acid sequence 100% identical to the amino acid sequence of SEQ ID NO: 186. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 180. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 184. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 180. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 184. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 180. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 184. In some embodiments, the antibody comprises a variable heavy chain (VH) region having an amino acid sequence with at least 95% (e.g., at least 95%, 96%, 97%, 98%, 99%, or more) identity to SEQ ID NO: 180. In some embodiments, the antibody comprises a variable light chain (VL) region having an amino acid sequence with at least 95% (e.g., at least 95%, 96%, 97%, 98%, 99%, or more) identity to SEQ ID NO: 184. In some embodiments, the antibody comprises a variable heavy chain (VH) region having an amino acid sequence 100% identical with the amino acid sequence of SEQ ID NO: 180. In some embodiments, the antibody comprises a variable light chain (VL) region having an amino acid sequence 100% identical with the amino acid sequence of SEQ ID NO: 184. In some embodiments, the antibody comprises: (a) a complementarity determining region (CDR) H1 having an amino acid sequence 100% identical to the amino acid sequence of SEQ ID NO: 181, a CDR H2 having an amino acid sequence 100% identical to the amino acid sequence of SEQ ID NO: 182, and a CDR H3 having an amino acid sequence 100% identical to the amino acid sequence of SEQ ID NO: 183; and (b) a complementarity determining region (CDR) L1 having 100% sequence identity to an amino acid sequence of SEQ ID NO: 185, a CDR L2 having an amino acid sequence 100% identical to the amino acid sequence of DVS, and a CDR L3 having an amino acid sequence 100% identical to the amino acid sequence of SEQ ID NO: 186. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 180 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 184. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 180 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 184. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 180 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 184. In some embodiments, the antibody comprises a variable heavy chain (VH) region having an amino acid sequence with at least 95% (e.g., at least 95%, 96%, 97%, 98%, 99%, or more) identity to SEQ ID NO: 180 and a variable light chain (VL) region having an amino acid sequence with at least 95% (e.g., at least 95%, 96%, 97%, 98%, 99%, or more) identity to SEQ ID NO: 184. In some embodiments, the antibody comprises a variable heavy chain (VH) region having an amino acid sequence 100% identical with the amino acid sequence of SEQ ID NO: 180 and a variable light chain (VL) region having an amino acid sequence 100% identical with the amino acid sequence of SEQ ID NO: 184. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 180 and comprising therein a complementarity determining region (CDR) H1 having an amino acid sequence 100% identical to the amino acid sequence of SEQ ID NO: 181, a CDR H2 having an amino acid sequence 100% identical to the amino acid sequence of SEQ ID NO: 182, and a CDR H3 having an amino acid sequence 100% identical to the amino acid sequence of SEQ ID NO: 183. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 184 and comprising therein a complementarity determining region (CDR) L1 having 100% sequence identity to an amino acid sequence of SEQ ID NO: 185, a CDR L2 having an amino acid sequence 100% identical to the amino acid sequence of DVS, and a CDR L3 having an amino acid sequence 100% identical to the amino acid sequence of SEQ ID NO: 186. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 180 and comprising therein a complementarity determining region (CDR) H1 having an amino acid sequence 100% identical to the amino acid sequence of SEQ ID NO: 181, a CDR H2 having an amino acid sequence 100% identical to the amino acid sequence of SEQ ID NO: 182, and a CDR H3 having an amino acid sequence 100% identical to the amino acid sequence of SEQ ID NO: 183; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 184 and comprising therein a complementarity determining region (CDR) L1 having 100% sequence identity to an amino acid sequence of SEQ ID NO: 185, a CDR L2 having an amino acid sequence 100% identical to the amino acid sequence of DVS, and a CDR L3 having an amino acid sequence 100% identical to the amino acid sequence of SEQ ID NO: 186.

In some embodiments, the antibody comprises a complementarity determining region (CDR) H1 having an amino acid sequence 100% identical to the amino acid sequence of SEQ ID NO: 188, a CDR H2 having an amino acid sequence 100% identical to the amino acid sequence of SEQ ID NO: 189, and a CDR H3 having an amino acid sequence 100% identical to the amino acid sequence of SEQ ID NO: 190. In some embodiments, the antibody comprises a complementarity determining region (CDR) L1 having 100% sequence identity to an amino acid sequence of SEQ ID NO: 192, a CDR L2 having an amino acid sequence 100% identical to the amino acid sequence of DAT, and a CDR L3 having an amino acid sequence 100% identical to the amino acid sequence of SEQ ID NO: 193. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 187. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 191. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 187. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 191. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 187. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 191. In some embodiments, the antibody comprises a variable heavy chain (VH) region having an amino acid sequence with at least 95% (e.g., at least 95%, 96%, 97%, 98%, 99%, or more) identity to SEQ ID NO: 187. In some embodiments, the antibody comprises a variable light chain (VL) region having an amino acid sequence with at least 95% (e.g., at least 95%, 96%, 97%, 98%, 99%, or more) identity to SEQ ID NO: 191. In some embodiments, the antibody comprises a variable heavy chain (VH) region having an amino acid sequence 100% identical with the amino acid sequence of SEQ ID NO: 187. In some embodiments, the antibody comprises a variable light chain (VL) region having an amino acid sequence 100% identical with the amino acid sequence of SEQ ID NO: 191. In some embodiments, the antibody comprises: (a) a complementarity determining region (CDR) H1 having an amino acid sequence 100% identical to the amino acid sequence of SEQ ID NO: 188, a CDR H2 having an amino acid sequence 100% identical to the amino acid sequence of SEQ ID NO: 189, and a CDR H3 having an amino acid sequence 100% identical to the amino acid sequence of SEQ ID NO: 190; and (b) a complementarity determining region (CDR) L1 having 100% sequence identity to an amino acid sequence of SEQ ID NO: 192, a CDR L2 having an amino acid sequence 100% identical to the amino acid sequence of DAT, and a CDR L3 having an amino acid sequence 100% identical to the amino acid sequence of SEQ ID NO: 193. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 187 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 191. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 187 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 191. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 187 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 191. In some embodiments, the antibody comprises a variable heavy chain (VH) region having an amino acid sequence with at least 95% (e.g., at least 95%, 96%, 97%, 98%, 99%, or more) identity to SEQ ID NO: 187 and a variable light chain (VL) region having an amino acid sequence with at least 95% (e.g., at least 95%, 96%, 97%, 98%, 99%, or more) identity to SEQ ID NO: 191. In some embodiments, the antibody comprises a variable heavy chain (VH) region having an amino acid sequence 100% identical with the amino acid sequence of SEQ ID NO: 187 and a variable light chain (VL) region having an amino acid sequence 100% identical with the amino acid sequence of SEQ ID NO: 191. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 187 and comprising therein a complementarity determining region (CDR) H1 having an amino acid sequence 100% identical to the amino acid sequence of SEQ ID NO: 188, a CDR H2 having an amino acid sequence 100% identical to the amino acid sequence of SEQ ID NO: 189, and a CDR H3 having an amino acid sequence 100% identical to the amino acid sequence of SEQ ID NO: 190. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 191 and comprising therein a complementarity determining region (CDR) L1 having 100% sequence identity to an amino acid sequence of SEQ ID NO: 192, a CDR L2 having an amino acid sequence 100% identical to the amino acid sequence of DAT, and a CDR L3 having an amino acid sequence 100% identical to the amino acid sequence of SEQ ID NO: 193. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 187 and comprising therein a complementarity determining region (CDR) H1 having an amino acid sequence 100% identical to the amino acid sequence of SEQ ID NO: 188, a CDR H2 having an amino acid sequence 100% identical to the amino acid sequence of SEQ ID NO: 189, and a CDR H3 having an amino acid sequence 100% identical to the amino acid sequence of SEQ ID NO: 190; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 191 and comprising therein a complementarity determining region (CDR) L1 having 100% sequence identity to an amino acid sequence of SEQ ID NO: 192, a CDR L2 having an amino acid sequence 100% identical to the amino acid sequence of DAT, and a CDR L3 having an amino acid sequence 100% identical to the amino acid sequence of SEQ ID NO: 193.

In some embodiments, the antibody comprises a complementarity determining region (CDR) H1 having an amino acid sequence 100% identical to the amino acid sequence of SEQ ID NO: 195, a CDR H2 having an amino acid sequence 100% identical to the amino acid sequence of SEQ ID NO: 196, and a CDR H3 having an amino acid sequence 100% identical to the amino acid sequence of SEQ ID NO: 197. In some embodiments, the antibody comprises a complementarity determining region (CDR) L1 having 100% sequence identity to an amino acid sequence of SEQ ID NO: 199, a CDR L2 having an amino acid sequence 100% identical to the amino acid sequence of DAS, and a CDR L3 having an amino acid sequence 100% identical to the amino acid sequence of SEQ ID NO: 200. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 194. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 198. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 194. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 198. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 194. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 198. In some embodiments, the antibody comprises a variable heavy chain (VH) region having an amino acid sequence with at least 95% (e.g., at least 95%, 96%, 97%, 98%, 99%, or more) identity to SEQ ID NO: 194. In some embodiments, the antibody comprises a variable light chain (VL) region having an amino acid sequence with at least 95% (e.g., at least 95%, 96%, 97%, 98%, 99%, or more) identity to SEQ ID NO: 198. In some embodiments, the antibody comprises a variable heavy chain (VH) region having an amino acid sequence 100% identical with the amino acid sequence of SEQ ID NO: 194. In some embodiments, the antibody comprises a variable light chain (VL) region having an amino acid sequence 100% identical with the amino acid sequence of SEQ ID NO: 198. In some embodiments, the antibody comprises: (a) a complementarity determining region (CDR) H1 having an amino acid sequence 100% identical to the amino acid sequence of SEQ ID NO: 195, a CDR H2 having an amino acid sequence 100% identical to the amino acid sequence of SEQ ID NO: 196, and a CDR H3 having an amino acid sequence 100% identical to the amino acid sequence of SEQ ID NO: 197; and (b) a complementarity determining region (CDR) L1 having 100% sequence identity to an amino acid sequence of SEQ ID NO: 199, a CDR L2 having an amino acid sequence 100% identical to the amino acid sequence of DAS, and a CDR L3 having an amino acid sequence 100% identical to the amino acid sequence of SEQ ID NO: 200. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 194 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 198. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 194 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 198. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 194 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 198. In some embodiments, the antibody comprises a variable heavy chain (VH) region having an amino acid sequence with at least 95% (e.g., at least 95%, 96%, 97%, 98%, 99%, or more) identity to SEQ ID NO: 194 and a variable light chain (VL) region having an amino acid sequence with at least 95% (e.g., at least 95%, 96%, 97%, 98%, 99%, or more) identity to SEQ ID NO: 198. In some embodiments, the antibody comprises a variable heavy chain (VH) region having an amino acid sequence 100% identical with the amino acid sequence of SEQ ID NO: 194 and a variable light chain (VL) region having an amino acid sequence 100% identical with the amino acid sequence of SEQ ID NO: 198. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 194 and comprising therein a complementarity determining region (CDR) H1 having an amino acid sequence 100% identical to the amino acid sequence of SEQ ID NO: 195, a CDR H2 having an amino acid sequence 100% identical to the amino acid sequence of SEQ ID NO: 196, and a CDR H3 having an amino acid sequence 100% identical to the amino acid sequence of SEQ ID NO: 197. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 198 and comprising therein a complementarity determining region (CDR) L1 having 100% sequence identity to an amino acid sequence of SEQ ID NO: 199, a CDR L2 having an amino acid sequence 100% identical to the amino acid sequence of DAS, and a CDR L3 having an amino acid sequence 100% identical to the amino acid sequence of SEQ ID NO: 200. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 194 and comprising therein a complementarity determining region (CDR) H1 having an amino acid sequence 100% identical to the amino acid sequence of SEQ ID NO: 195, a CDR H2 having an amino acid sequence 100% identical to the amino acid sequence of SEQ ID NO: 196, and a CDR H3 having an amino acid sequence 100% identical to the amino acid sequence of SEQ ID NO: 197; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 198 and comprising therein a complementarity determining region (CDR) L1 having 100% sequence identity to an amino acid sequence of SEQ ID NO: 199, a CDR L2 having an amino acid sequence 100% identical to the amino acid sequence of DAS, and a CDR L3 having an amino acid sequence 100% identical to the amino acid sequence of SEQ ID NO: 200.

In some embodiments, the antibody comprises a complementarity determining region (CDR) H1 having an amino acid sequence 100% identical to the amino acid sequence of SEQ ID NO: 202, a CDR H2 having an amino acid sequence 100% identical to the amino acid sequence of SEQ ID NO: 203, and a CDR H3 having an amino acid sequence 100% identical to the amino acid sequence of SEQ ID NO: 204. In some embodiments, the antibody comprises a complementarity determining region (CDR) L1 having 100% sequence identity to an amino acid sequence of SEQ ID NO: 206, a CDR L2 having an amino acid sequence 100% identical to the amino acid sequence of GVD, and a CDR L3 having an amino acid sequence 100% identical to the amino acid sequence of SEQ ID NO: 207. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 201. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 205. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 201. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 205. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 201. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 205. In some embodiments, the antibody comprises a variable heavy chain (VH) region having an amino acid sequence with at least 95% (e.g., at least 95%, 96%, 97%, 98%, 99%, or more) identity to SEQ ID NO: 201. In some embodiments, the antibody comprises a variable light chain (VL) region having an amino acid sequence with at least 95% (e.g., at least 95%, 96%, 97%, 98%, 99%, or more) identity to SEQ ID NO: 205. In some embodiments, the antibody comprises a variable heavy chain (VH) region having an amino acid sequence 100% identical with the amino acid sequence of SEQ ID NO: 201. In some embodiments, the antibody comprises a variable light chain (VL) region having an amino acid sequence 100% identical with the amino acid sequence of SEQ ID NO: 205. In some embodiments, the antibody comprises: (a) a complementarity determining region (CDR) H1 having an amino acid sequence 100% identical to the amino acid sequence of SEQ ID NO: 202, a CDR H2 having an amino acid sequence 100% identical to the amino acid sequence of SEQ ID NO: 203, and a CDR H3 having an amino acid sequence 100% identical to the amino acid sequence of SEQ ID NO: 204; and (b) a complementarity determining region (CDR) L1 having 100% sequence identity to an amino acid sequence of SEQ ID NO: 206, a CDR L2 having an amino acid sequence 100% identical to the amino acid sequence of GVD, and a CDR L3 having an amino acid sequence 100% identical to the amino acid sequence of SEQ ID NO: 207. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 201 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 205. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 201 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 205. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 201 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 205. In some embodiments, the antibody comprises a variable heavy chain (VH) region having an amino acid sequence with at least 95% (e.g., at least 95%, 96%, 97%, 98%, 99%, or more) identity to SEQ ID NO: 201 and a variable light chain (VL) region having an amino acid sequence with at least 95% (e.g., at least 95%, 96%, 97%, 98%, 99%, or more) identity to SEQ ID NO: 205. In some embodiments, the antibody comprises a variable heavy chain (VH) region having an amino acid sequence 100% identical with the amino acid sequence of SEQ ID NO: 201 and a variable light chain (VL) region having an amino acid sequence 100% identical with the amino acid sequence of SEQ ID NO: 205. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 201 and comprising therein a complementarity determining region (CDR) H1 having an amino acid sequence 100% identical to the amino acid sequence of SEQ ID NO: 202, a CDR H2 having an amino acid sequence 100% identical to the amino acid sequence of SEQ ID NO: 203, and a CDR H3 having an amino acid sequence 100% identical to the amino acid sequence of SEQ ID NO: 204. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 205 and comprising therein a complementarity determining region (CDR) L1 having 100% sequence identity to an amino acid sequence of SEQ ID NO: 206, a CDR L2 having an amino acid sequence 100% identical to the amino acid sequence of GVD, and a CDR L3 having an amino acid sequence 100% identical to the amino acid sequence of SEQ ID NO: 207. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 201 and comprising therein a complementarity determining region (CDR) H1 having an amino acid sequence 100% identical to the amino acid sequence of SEQ ID NO: 202, a CDR H2 having an amino acid sequence 100% identical to the amino acid sequence of SEQ ID NO: 203, and a CDR H3 having an amino acid sequence 100% identical to the amino acid sequence of SEQ ID NO: 204; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 205 and comprising therein a complementarity determining region (CDR) L1 having 100% sequence identity to an amino acid sequence of SEQ ID NO: 206, a CDR L2 having an amino acid sequence 100% identical to the amino acid sequence of GVD, and a CDR L3 having an amino acid sequence 100% identical to the amino acid sequence of SEQ ID NO: 207.

In some embodiments, the antibody comprises a complementarity determining region (CDR) H1 having an amino acid sequence 100% identical to the amino acid sequence of SEQ ID NO: 209, a CDR H2 having an amino acid sequence 100% identical to the amino acid sequence of SEQ ID NO: 210, and a CDR H3 having an amino acid sequence 100% identical to the amino acid sequence of SEQ ID NO: 211. In some embodiments, the antibody comprises a complementarity determining region (CDR) L1 having 100% sequence identity to an amino acid sequence of SEQ ID NO: 213, a CDR L2 having an amino acid sequence 100% identical to the amino acid sequence of DAS, and a CDR L3 having an amino acid sequence 100% identical to the amino acid sequence of SEQ ID NO: 214. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 208. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 212. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 208. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 212. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 208. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 212. In some embodiments, the antibody comprises a variable heavy chain (VH) region having an amino acid sequence with at least 95% (e.g., at least 95%, 96%, 97%, 98%, 99%, or more) identity to SEQ ID NO: 208. In some embodiments, the antibody comprises a variable light chain (VL) region having an amino acid sequence with at least 95% (e.g., at least 95%, 96%, 97%, 98%, 99%, or more) identity to SEQ ID NO: 212. In some embodiments, the antibody comprises a variable heavy chain (VH) region having an amino acid sequence 100% identical with the amino acid sequence of SEQ ID NO: 208. In some embodiments, the antibody comprises a variable light chain (VL) region having an amino acid sequence 100% identical with the amino acid sequence of SEQ ID NO: 212. In some embodiments, the antibody comprises: (a) a complementarity determining region (CDR) H1 having an amino acid sequence 100% identical to the amino acid sequence of SEQ ID NO: 209, a CDR H2 having an amino acid sequence 100% identical to the amino acid sequence of SEQ ID NO: 210, and a CDR H3 having an amino acid sequence 100% identical to the amino acid sequence of SEQ ID NO: 211; and (b) a complementarity determining region (CDR) L1 having 100% sequence identity to an amino acid sequence of SEQ ID NO: 213, a CDR L2 having an amino acid sequence 100% identical to the amino acid sequence of DAS, and a CDR L3 having an amino acid sequence 100% identical to the amino acid sequence of SEQ ID NO: 214. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 208 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 212. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 208 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 212. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 208 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 212. In some embodiments, the antibody comprises a variable heavy chain (VH) region having an amino acid sequence with at least 95% (e.g., at least 95%, 96%, 97%, 98%, 99%, or more) identity to SEQ ID NO: 208 and a variable light chain (VL) region having an amino acid sequence with at least 95% (e.g., at least 95%, 96%, 97%, 98%, 99%, or more) identity to SEQ ID NO: 212. In some embodiments, the antibody comprises a variable heavy chain (VH) region having an amino acid sequence 100% identical with the amino acid sequence of SEQ ID NO: 208 and a variable light chain (VL) region having an amino acid sequence 100% identical with the amino acid sequence of SEQ ID NO: 212. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 208 and comprising therein a complementarity determining region (CDR) H1 having an amino acid sequence 100% identical to the amino acid sequence of SEQ ID NO: 209, a CDR H2 having an amino acid sequence 100% identical to the amino acid sequence of SEQ ID NO: 210, and a CDR H3 having an amino acid sequence 100% identical to the amino acid sequence of SEQ ID NO: 211. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 212 and comprising therein a complementarity determining region (CDR) L1 having 100% sequence identity to an amino acid sequence of SEQ ID NO: 213, a CDR L2 having an amino acid sequence 100% identical to the amino acid sequence of DAS, and a CDR L3 having an amino acid sequence 100% identical to the amino acid sequence of SEQ ID NO: 214. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 208 and comprising therein a complementarity determining region (CDR) H1 having an amino acid sequence 100% identical to the amino acid sequence of SEQ ID NO: 209, a CDR H2 having an amino acid sequence 100% identical to the amino acid sequence of SEQ ID NO: 210, and a CDR H3 having an amino acid sequence 100% identical to the amino acid sequence of SEQ ID NO: 211; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 212 and comprising therein a complementarity determining region (CDR) L1 having 100% sequence identity to an amino acid sequence of SEQ ID NO: 213, a CDR L2 having an amino acid sequence 100% identical to the amino acid sequence of DAS, and a CDR L3 having an amino acid sequence 100% identical to the amino acid sequence of SEQ ID NO: 214.

In some embodiments, the antibody comprises a complementarity determining region (CDR) H1 having an amino acid sequence 100% identical to the amino acid sequence of SEQ ID NO: 216, a CDR H2 having an amino acid sequence 100% identical to the amino acid sequence of SEQ ID NO: 217, and a CDR H3 having an amino acid sequence 100% identical to the amino acid sequence of SEQ ID NO: 218. In some embodiments, the antibody comprises a complementarity determining region (CDR) L1 having 100% sequence identity to an amino acid sequence of SEQ ID NO: 220, a CDR L2 having an amino acid sequence 100% identical to the amino acid sequence of GAT, and a CDR L3 having an amino acid sequence 100% identical to the amino acid sequence of SEQ ID NO: 221. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 215. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 219. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 215. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 219. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 215. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 219. In some embodiments, the antibody comprises a variable heavy chain (VH) region having an amino acid sequence with at least 95% (e.g., at least 95%, 96%, 97%, 98%, 99%, or more) identity to SEQ ID NO: 215. In some embodiments, the antibody comprises a variable light chain (VL) region having an amino acid sequence with at least 95% (e.g., at least 95%, 96%, 97%, 98%, 99%, or more) identity to SEQ ID NO: 219. In some embodiments, the antibody comprises a variable heavy chain (VH) region having an amino acid sequence 100% identical with the amino acid sequence of SEQ ID NO: 215. In some embodiments, the antibody comprises a variable light chain (VL) region having an amino acid sequence 100% identical with the amino acid sequence of SEQ ID NO: 219. In some embodiments, the antibody comprises: (a) a complementarity determining region (CDR) H1 having an amino acid sequence 100% identical to the amino acid sequence of SEQ ID NO: 216, a CDR H2 having an amino acid sequence 100% identical to the amino acid sequence of SEQ ID NO: 217, and a CDR H3 having an amino acid sequence 100% identical to the amino acid sequence of SEQ ID NO: 218; and (b) a complementarity determining region (CDR) L1 having 100% sequence identity to an amino acid sequence of SEQ ID NO: 220, a CDR L2 having an amino acid sequence 100% identical to the amino acid sequence of GAT, and a CDR L3 having an amino acid sequence 100% identical to the amino acid sequence of SEQ ID NO: 221. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 215 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 219. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 215 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 219. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 215 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 219. In some embodiments, the antibody comprises a variable heavy chain (VH) region having an amino acid sequence with at least 95% (e.g., at least 95%, 96%, 97%, 98%, 99%, or more) identity to SEQ ID NO: 215 and a variable light chain (VL) region having an amino acid sequence with at least 95% (e.g., at least 95%, 96%, 97%, 98%, 99%, or more) identity to SEQ ID NO: 219. In some embodiments, the antibody comprises a variable heavy chain (VH) region having an amino acid sequence 100% identical with the amino acid sequence of SEQ ID NO: 215 and a variable light chain (VL) region having an amino acid sequence 100% identical with the amino acid sequence of SEQ ID NO: 219. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 215 and comprising therein a complementarity determining region (CDR) H1 having an amino acid sequence 100% identical to the amino acid sequence of SEQ ID NO: 216, a CDR H2 having an amino acid sequence 100% identical to the amino acid sequence of SEQ ID NO: 217, and a CDR H3 having an amino acid sequence 100% identical to the amino acid sequence of SEQ ID NO: 218. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 219 and comprising therein a complementarity determining region (CDR) L1 having 100% sequence identity to an amino acid sequence of SEQ ID NO: 220, a CDR L2 having an amino acid sequence 100% identical to the amino acid sequence of GAT, and a CDR L3 having an amino acid sequence 100% identical to the amino acid sequence of SEQ ID NO: 221. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 215 and comprising therein a complementarity determining region (CDR) H1 having an amino acid sequence 100% identical to the amino acid sequence of SEQ ID NO: 216, a CDR H2 having an amino acid sequence 100% identical to the amino acid sequence of SEQ ID NO: 217, and a CDR H3 having an amino acid sequence 100% identical to the amino acid sequence of SEQ ID NO: 218; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 219 and comprising therein a complementarity determining region (CDR) L1 having 100% sequence identity to an amino acid sequence of SEQ ID NO: 220, a CDR L2 having an amino acid sequence 100% identical to the amino acid sequence of GAT, and a CDR L3 having an amino acid sequence 100% identical to the amino acid sequence of SEQ ID NO: 221.

In some embodiments, the antibody comprises a complementarity determining region (CDR) H1 having an amino acid sequence 100% identical to the amino acid sequence of SEQ ID NO: 223, a CDR H2 having an amino acid sequence 100% identical to the amino acid sequence of SEQ ID NO: 224, and a CDR H3 having an amino acid sequence 100% identical to the amino acid sequence of SEQ ID NO: 225. In some embodiments, the antibody comprises a complementarity determining region (CDR) L1 having 100% sequence identity to an amino acid sequence of SEQ ID NO: 227, a CDR L2 having an amino acid sequence 100% identical to the amino acid sequence of DAS, and a CDR L3 having an amino acid sequence 100% identical to the amino acid sequence of SEQ ID NO: 228. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 222. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 226. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 222. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 226. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 222. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 226. In some embodiments, the antibody comprises a variable heavy chain (VH) region having an amino acid sequence with at least 95% (e.g., at least 95%, 96%, 97%, 98%, 99%, or more) identity to SEQ ID NO: 222. In some embodiments, the antibody comprises a variable light chain (VL) region having an amino acid sequence with at least 95% (e.g., at least 95%, 96%, 97%, 98%, 99%, or more) identity to SEQ ID NO: 226. In some embodiments, the antibody comprises a variable heavy chain (VH) region having an amino acid sequence 100% identical with the amino acid sequence of SEQ ID NO: 222. In some embodiments, the antibody comprises a variable light chain (VL) region having an amino acid sequence 100% identical with the amino acid sequence of SEQ ID NO: 226. In some embodiments, the antibody comprises: (a) a complementarity determining region (CDR) H1 having an amino acid sequence 100% identical to the amino acid sequence of SEQ ID NO: 223, a CDR H2 having an amino acid sequence 100% identical to the amino acid sequence of SEQ ID NO: 224, and a CDR H3 having an amino acid sequence 100% identical to the amino acid sequence of SEQ ID NO: 225; and (b) a complementarity determining region (CDR) L1 having 100% sequence identity to an amino acid sequence of SEQ ID NO: 227, a CDR L2 having an amino acid sequence 100% identical to the amino acid sequence of DAS, and a CDR L3 having an amino acid sequence 100% identical to the amino acid sequence of SEQ ID NO: 228. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 222 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 226. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 222 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 226. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 222 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 226. In some embodiments, the antibody comprises a variable heavy chain (VH) region having an amino acid sequence with at least 95% (e.g., at least 95%, 96%, 97%, 98%, 99%, or more) identity to SEQ ID NO: 222 and a variable light chain (VL) region having an amino acid sequence with at least 95% (e.g., at least 95%, 96%, 97%, 98%, 99%, or more) identity to SEQ ID NO: 226. In some embodiments, the antibody comprises a variable heavy chain (VH) region having an amino acid sequence 100% identical with the amino acid sequence of SEQ ID NO: 222 and a variable light chain (VL) region having an amino acid sequence 100% identical with the amino acid sequence of SEQ ID NO: 226. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 222 and comprising therein a complementarity determining region (CDR) H1 having an amino acid sequence 100% identical to the amino acid sequence of SEQ ID NO: 223, a CDR H2 having an amino acid sequence 100% identical to the amino acid sequence of SEQ ID NO: 224, and a CDR H3 having an amino acid sequence 100% identical to the amino acid sequence of SEQ ID NO: 225. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 226 and comprising therein a complementarity determining region (CDR) L1 having 100% sequence identity to an amino acid sequence of SEQ ID NO: 227, a CDR L2 having an amino acid sequence 100% identical to the amino acid sequence of DAS, and a CDR L3 having an amino acid sequence 100% identical to the amino acid sequence of SEQ ID NO: 228. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 222 and comprising therein a complementarity determining region (CDR) H1 having an amino acid sequence 100% identical to the amino acid sequence of SEQ ID NO: 223, a CDR H2 having an amino acid sequence 100% identical to the amino acid sequence of SEQ ID NO: 224, and a CDR H3 having an amino acid sequence 100% identical to the amino acid sequence of SEQ ID NO: 225; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 226 and comprising therein a complementarity determining region (CDR) L1 having 100% sequence identity to an amino acid sequence of SEQ ID NO: 227, a CDR L2 having an amino acid sequence 100% identical to the amino acid sequence of DAS, and a CDR L3 having an amino acid sequence 100% identical to the amino acid sequence of SEQ ID NO: 228.

In some embodiments, the antibody comprises a complementarity determining region (CDR) H1 having an amino acid sequence 100% identical to the amino acid sequence of SEQ ID NO: 230, a CDR H2 having an amino acid sequence 100% identical to the amino acid sequence of SEQ ID NO: 231, and a CDR H3 having an amino acid sequence 100% identical to the amino acid sequence of SEQ ID NO: 232. In some embodiments, the antibody comprises a complementarity determining region (CDR) L1 having 100% sequence identity to an amino acid sequence of SEQ ID NO: 234, a CDR L2 having an amino acid sequence 100% identical to the amino acid sequence of DAS, and a CDR L3 having an amino acid sequence 100% identical to the amino acid sequence of SEQ ID NO: 235. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 229. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 233. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 229. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 233. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 229. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 233. In some embodiments, the antibody comprises a variable heavy chain (VH) region having an amino acid sequence with at least 95% (e.g., at least 95%, 96%, 97%, 98%, 99%, or more) identity to SEQ ID NO: 229. In some embodiments, the antibody comprises a variable light chain (VL) region having an amino acid sequence with at least 95% (e.g., at least 95%, 96%, 97%, 98%, 99%, or more) identity to SEQ ID NO: 233. In some embodiments, the antibody comprises a variable heavy chain (VH) region having an amino acid sequence 100% identical with the amino acid sequence of SEQ ID NO: 229. In some embodiments, the antibody comprises a variable light chain (VL) region having an amino acid sequence 100% identical with the amino acid sequence of SEQ ID NO: 233. In some embodiments, the antibody comprises: (a) a complementarity determining region (CDR) H1 having an amino acid sequence 100% identical to the amino acid sequence of SEQ ID NO: 230, a CDR H2 having an amino acid sequence 100% identical to the amino acid sequence of SEQ ID NO: 231, and a CDR H3 having an amino acid sequence 100% identical to the amino acid sequence of SEQ ID NO: 232; and (b) a complementarity determining region (CDR) L1 having 100% sequence identity to an amino acid sequence of SEQ ID NO: 234, a CDR L2 having an amino acid sequence 100% identical to the amino acid sequence of DAS, and a CDR L3 having an amino acid sequence 100% identical to the amino acid sequence of SEQ ID NO: 235. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 229 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 233. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 229 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 233. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 229 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 233. In some embodiments, the antibody comprises a variable heavy chain (VH) region having an amino acid sequence with at least 95% (e.g., at least 95%, 96%, 97%, 98%, 99%, or more) identity to SEQ ID NO: 229 and a variable light chain (VL) region having an amino acid sequence with at least 95% (e.g., at least 95%, 96%, 97%, 98%, 99%, or more) identity to SEQ ID NO: 233. In some embodiments, the antibody comprises a variable heavy chain (VH) region having an amino acid sequence 100% identical with the amino acid sequence of SEQ ID NO: 229 and a variable light chain (VL) region having an amino acid sequence 100% identical with the amino acid sequence of SEQ ID NO: 233. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 229 and comprising therein a complementarity determining region (CDR) H1 having an amino acid sequence 100% identical to the amino acid sequence of SEQ ID NO: 230, a CDR H2 having an amino acid sequence 100% identical to the amino acid sequence of SEQ ID NO: 231, and a CDR H3 having an amino acid sequence 100% identical to the amino acid sequence of SEQ ID NO: 232. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 233 and comprising therein a complementarity determining region (CDR) L1 having 100% sequence identity to an amino acid sequence of SEQ ID NO: 234, a CDR L2 having an amino acid sequence 100% identical to the amino acid sequence of DAS, and a CDR L3 having an amino acid sequence 100% identical to the amino acid sequence of SEQ ID NO: 235. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 229 and comprising therein a complementarity determining region (CDR) H1 having an amino acid sequence 100% identical to the amino acid sequence of SEQ ID NO: 230, a CDR H2 having an amino acid sequence 100% identical to the amino acid sequence of SEQ ID NO: 231, and a CDR H3 having an amino acid sequence 100% identical to the amino acid sequence of SEQ ID NO: 232; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 233 and comprising therein a complementarity determining region (CDR) L1 having 100% sequence identity to an amino acid sequence of SEQ ID NO: 234, a CDR L2 having an amino acid sequence 100% identical to the amino acid sequence of DAS, and a CDR L3 having an amino acid sequence 100% identical to the amino acid sequence of SEQ ID NO: 235.

In some embodiments, the antibody comprises a complementarity determining region (CDR) H1 having an amino acid sequence 100% identical to the amino acid sequence of SEQ ID NO: 237, a CDR H2 having an amino acid sequence 100% identical to the amino acid sequence of SEQ ID NO: 238, and a CDR H3 having an amino acid sequence 100% identical to the amino acid sequence of SEQ ID NO: 239. In some embodiments, the antibody comprises a complementarity determining region (CDR) L1 having 100% sequence identity to an amino acid sequence of SEQ ID NO: 241, a CDR L2 having an amino acid sequence 100% identical to the amino acid sequence of GAS, and a CDR L3 having an amino acid sequence 100% identical to the amino acid sequence of SEQ ID NO: 242. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 236. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 240. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 236. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 240. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 236. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 240. In some embodiments, the antibody comprises a variable heavy chain (VH) region having an amino acid sequence with at least 95% (e.g., at least 95%, 96%, 97%, 98%, 99%, or more) identity to SEQ ID NO: 236. In some embodiments, the antibody comprises a variable light chain (VL) region having an amino acid sequence with at least 95% (e.g., at least 95%, 96%, 97%, 98%, 99%, or more) identity to SEQ ID NO: 240. In some embodiments, the antibody comprises a variable heavy chain (VH) region having an amino acid sequence 100% identical with the amino acid sequence of SEQ ID NO: 236. In some embodiments, the antibody comprises a variable light chain (VL) region having an amino acid sequence 100% identical with the amino acid sequence of SEQ ID NO: 240. In some embodiments, the antibody comprises: (a) a complementarity determining SEQ ID NO: 237, a CDR H2 having an amino acid sequence 100% identical to the amino acid sequence of SEQ ID NO: 238, and a CDR H3 having an amino acid sequence 100% identical to the amino acid sequence of SEQ ID NO: 239; and (b) a complementarity determining region (CDR) L1 having 100% sequence identity to an amino acid sequence of SEQ ID NO: 241, a CDR L2 having an amino acid sequence 100% identical to the amino acid sequence of GAS, and a CDR L3 having an amino acid sequence 100% identical to the amino acid sequence of SEQ ID NO: 242. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 236 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 240. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 236 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 240. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 236 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 240. In some embodiments, the antibody comprises a variable heavy chain (VH) region having an amino acid sequence with at least 95% (e.g., at least 95%, 96%, 97%, 98%, 99%, or more) identity to SEQ ID NO: 236 and a variable light chain (VL) region having an amino acid sequence with at least 95% (e.g., at least 95%, 96%, 97%, 98%, 99%, or more) identity to SEQ ID NO: 240. In some embodiments, the antibody comprises a variable heavy chain (VH) region having an amino acid sequence 100% identical with the amino acid sequence of SEQ ID NO: 236 and a variable light chain (VL) region having an amino acid sequence 100% identical with the amino acid sequence of SEQ ID NO: 240. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 236 and comprising therein a complementarity determining region (CDR) H1 having an amino acid sequence 100% identical to the amino acid sequence of SEQ ID NO: 237, a CDR H2 having an amino acid sequence 100% identical to the amino acid sequence of SEQ ID NO: 238, and a CDR H3 having an amino acid sequence 100% identical to the amino acid sequence of SEQ ID NO: 239. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 240 and comprising therein a complementarity determining region (CDR) L1 having 100% sequence identity to an amino acid sequence of SEQ ID NO: 241, a CDR L2 having an amino acid sequence 100% identical to the amino acid sequence of GAS, and a CDR L3 having an amino acid sequence 100% identical to the amino acid sequence of SEQ ID NO: 242. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 236 and comprising therein a complementarity determining region (CDR) H1 having an amino acid sequence 100% identical to the amino acid sequence of SEQ ID NO: 237, a CDR H2 having an amino acid sequence 100% identical to the amino acid sequence of SEQ ID NO: 238, and a CDR H3 having an amino acid sequence 100% identical to the amino acid sequence of SEQ ID NO: 239; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 240 and comprising therein a complementarity determining region (CDR) L1 having 100% sequence identity to an amino acid sequence of SEQ ID NO: 241, a CDR L2 having an amino acid sequence 100% identical to the amino acid sequence of GAS, and a CDR L3 having an amino acid sequence 100% identical to the amino acid sequence of SEQ ID NO: 242.

In some embodiments, the antibody comprises a complementarity determining region (CDR) H1 having an amino acid sequence 100% identical to the amino acid sequence of SEQ ID NO: 244, a CDR H2 having an amino acid sequence 100% identical to the amino acid sequence of SEQ ID NO: 245, and a CDR H3 having an amino acid sequence 100% identical to the amino acid sequence of SEQ ID NO: 246. In some embodiments, the antibody comprises a complementarity determining region (CDR) L1 having 100% sequence identity to an amino acid sequence of SEQ ID NO: 248, a CDR L2 having an amino acid sequence 100% identical to the amino acid sequence of GAS, and a CDR L3 having an amino acid sequence 100% identical to the amino acid sequence of SEQ ID NO: 249. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 243. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 247. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 243. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 247. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 243. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 247. In some embodiments, the antibody comprises a variable heavy chain (VH) region having an amino acid sequence with at least 95% (e.g., at least 95%, 96%, 97%, 98%, 99%, or more) identity to SEQ ID NO: 243. In some embodiments, the antibody comprises a variable light chain (VL) region having an amino acid sequence with at least 95% (e.g., at least 95%, 96%, 97%, 98%, 99%, or more) identity to SEQ ID NO: 247. In some embodiments, the antibody comprises a variable heavy chain (VH) region having an amino acid sequence 100% identical with the amino acid sequence of SEQ ID NO: 243. In some embodiments, the antibody comprises a variable light chain (VL) region having an amino acid sequence 100% identical with the amino acid sequence of SEQ ID NO: 247. In some embodiments, the antibody comprises: (a) a complementarity determining region (CDR) H1 having an amino acid sequence 100% identical to the amino acid sequence of SEQ ID NO: 244, a CDR H2 having an amino acid sequence 100% identical to the amino acid sequence of SEQ ID NO: 245, and a CDR H3 having an amino acid sequence 100% identical to the amino acid sequence of SEQ ID NO: 246; and (b) a complementarity determining region (CDR) L1 having 100% sequence identity to an amino acid sequence of SEQ ID NO: 248, a CDR L2 having an amino acid sequence 100% identical to the amino acid sequence of GAS, and a CDR L3 having an amino acid sequence 100% identical to the amino acid sequence of SEQ ID NO: 249. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 243 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 247. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 243 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 247. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 243 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 247. In some embodiments, the antibody comprises a variable heavy chain (VH) region having an amino acid sequence with at least 95% (e.g., at least 95%, 96%, 97%, 98%, 99%, or more) identity to SEQ ID NO: 243 and a variable light chain (VL) region having an amino acid sequence with at least 95% (e.g., at least 95%, 96%, 97%, 98%, 99%, or more) identity to SEQ ID NO: 247. In some embodiments, the antibody comprises a variable heavy chain (VH) region having an amino acid sequence 100% identical with the amino acid sequence of SEQ ID NO: 243 and a variable light chain (VL) region having an amino acid sequence 100% identical with the amino acid sequence of SEQ ID NO: 247. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 243 and comprising therein a complementarity determining region (CDR) H1 having an amino acid sequence 100% identical to the amino acid sequence of SEQ ID NO: 244, a CDR H2 having an amino acid sequence 100% identical to the amino acid sequence of SEQ ID NO: 245, and a CDR H3 having an amino acid sequence 100% identical to the amino acid sequence of SEQ ID NO: 246. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 247 and comprising therein a complementarity determining region (CDR) L1 having 100% sequence identity to an amino acid sequence of SEQ ID NO: 248, a CDR L2 having an amino acid sequence 100% identical to the amino acid sequence of GAS, and a CDR L3 having an amino acid sequence 100% identical to the amino acid sequence of SEQ ID NO: 249. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 243 and comprising therein a complementarity determining region (CDR) H1 having an amino acid sequence 100% identical to the amino acid sequence of SEQ ID NO: 244, a CDR H2 having an amino acid sequence 100% identical to the amino acid sequence of SEQ ID NO: 245, and a CDR H3 having an amino acid sequence 100% identical to the amino acid sequence of SEQ ID NO: 246; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 247 and comprising therein a complementarity determining region (CDR) L1 having 100% sequence identity to an amino acid sequence of SEQ ID NO: 248, a CDR L2 having an amino acid sequence 100% identical to the amino acid sequence of GAS, and a CDR L3 having an amino acid sequence 100% identical to the amino acid sequence of SEQ ID NO: 249. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 460. In some embodiments, the antibody comprises a second heavy chain region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 480. In some embodiments, the antibody comprises a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 470. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 460, a second heavy chain region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 480, and a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 470.

In some embodiments, the antibody comprises a complementarity determining region (CDR) H1 having an amino acid sequence 100% identical to the amino acid sequence of SEQ ID NO: 251, a CDR H2 having an amino acid sequence 100% identical to the amino acid sequence of SEQ ID NO: 252, and a CDR H3 having an amino acid sequence 100% identical to the amino acid sequence of SEQ ID NO: 253. In some embodiments, the antibody comprises a complementarity determining region (CDR) L1 having 100% sequence identity to an amino acid sequence of SEQ ID NO: 255, a CDR L2 having an amino acid sequence 100% identical to the amino acid sequence of GAT, and a CDR L3 having an amino acid sequence 100% identical to the amino acid sequence of SEQ ID NO: 256. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 250. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 254. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 250. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 254. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 250. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 254. In some embodiments, the antibody comprises a variable heavy chain (VH) region having an amino acid sequence with at least 95% (e.g., at least 95%, 96%, 97%, 98%, 99%, or more) identity to SEQ ID NO: 250. In some embodiments, the antibody comprises a variable light chain (VL) region having an amino acid sequence with at least 95% (e.g., at least 95%, 96%, 97%, 98%, 99%, or more) identity to SEQ ID NO: 254. In some embodiments, the antibody comprises a variable heavy chain (VH) region having an amino acid sequence 100% identical with the amino acid sequence of SEQ ID NO: 250. In some embodiments, the antibody comprises a variable light chain (VL) region having an amino acid sequence 100% identical with the amino acid sequence of SEQ ID NO: 254. In some embodiments, the antibody comprises: (a) a complementarity determining region (CDR) H1 having an amino acid sequence 100% identical to the amino acid sequence of SEQ ID NO: 251, a CDR H2 having an amino acid sequence 100% identical to the amino acid sequence of SEQ ID NO: 252, and a CDR H3 having an amino acid sequence 100% identical to the amino acid sequence of SEQ ID NO: 253; and (b) a complementarity determining region (CDR) L1 having 100% sequence identity to an amino acid sequence of SEQ ID NO: 255, a CDR L2 having an amino acid sequence 100% identical to the amino acid sequence of GAT, and a CDR L3 having an amino acid sequence 100% identical to the amino acid sequence of SEQ ID NO: 256. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 250 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 254. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 250 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 254. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 250 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 254. In some embodiments, the antibody comprises a variable heavy chain (VH) region having an amino acid sequence with at least 95% (e.g., at least 95%, 96%, 97%, 98%, 99%, or more) identity to SEQ ID NO: 250 and a variable light chain (VL) region having an amino acid sequence with at least 95% (e.g., at least 95%, 96%, 97%, 98%, 99%, or more) identity to SEQ ID NO: 254. In some embodiments, the antibody comprises a variable heavy chain (VH) region having an amino acid sequence 100% identical with the amino acid sequence of SEQ ID NO: 250 and a variable light chain (VL) region having an amino acid sequence 100% identical with the amino acid sequence of SEQ ID NO: 254. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 250 and comprising therein a complementarity determining region (CDR) H1 having an amino acid sequence 100% identical to the amino acid sequence of SEQ ID NO: 251, a CDR H2 having an amino acid sequence 100% identical to the amino acid sequence of SEQ ID NO: 252, and a CDR H3 having an amino acid sequence 100% identical to the amino acid sequence of SEQ ID NO: 253. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 254 and comprising therein a complementarity determining region (CDR) L1 having 100% sequence identity to an amino acid sequence of SEQ ID NO: 255, a CDR L2 having an amino acid sequence 100% identical to the amino acid sequence of GAT, and a CDR L3 having an amino acid sequence 100% identical to the amino acid sequence of SEQ ID NO: 256. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 250 and comprising therein a complementarity determining region (CDR) H1 having an amino acid sequence 100% identical to the amino acid sequence of SEQ ID NO: 251, a CDR H2 having an amino acid sequence 100% identical to the amino acid sequence of SEQ ID NO: 252, and a CDR H3 having an amino acid sequence 100% identical to the amino acid sequence of SEQ ID NO: 253; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 254 and comprising therein a complementarity determining region (CDR) L1 having 100% sequence identity to an amino acid sequence of SEQ ID NO: 255, a CDR L2 having an amino acid sequence 100% identical to the amino acid sequence of GAT, and a CDR L3 having an amino acid sequence 100% identical to the amino acid sequence of SEQ ID NO: 256.

In some embodiments, the antibody comprises a complementarity determining region (CDR) H1 having an amino acid sequence 100% identical to the amino acid sequence of SEQ ID NO: 258, a CDR H2 having an amino acid sequence 100% identical to the amino acid sequence of SEQ ID NO: 259, and a CDR H3 having an amino acid sequence 100% identical to the amino acid sequence of SEQ ID NO: 260. In some embodiments, the antibody comprises a complementarity determining region (CDR) L1 having 100% sequence identity to an amino acid sequence of SEQ ID NO: 262, a CDR L2 having an amino acid sequence 100% identical to the amino acid sequence of GAS, and a CDR L3 having an amino acid sequence 100% identical to the amino acid sequence of SEQ ID NO: 263. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 257. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 261. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 257. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 261. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 257. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 261. In some embodiments, the antibody comprises a variable heavy chain (VH) region having an amino acid sequence with at least 95% (e.g., at least 95%, 96%, 97%, 98%, 99%, or more) identity to SEQ ID NO: 257. In some embodiments, the antibody comprises a variable light chain (VL) region having an amino acid sequence with at least 95% (e.g., at least 95%, 96%, 97%, 98%, 99%, or more) identity to SEQ ID NO: 261. In some embodiments, the antibody comprises a variable heavy chain (VH) region having an amino acid sequence 100% identical with the amino acid sequence of SEQ ID NO: 257. In some embodiments, the antibody comprises a variable light chain (VL) region having an amino acid sequence 100% identical with the amino acid sequence of SEQ ID NO: 261. In some embodiments, the antibody comprises: (a) a complementarity determining region (CDR) H1 having an amino acid sequence 100% identical to the amino acid sequence of SEQ ID NO: 258, a CDR H2 having an amino acid sequence 100% identical to the amino acid sequence of SEQ ID NO: 259, and a CDR H3 having an amino acid sequence 100% identical to the amino acid sequence of SEQ ID NO: 260; and (b) a complementarity determining region (CDR) L1 having 100% sequence identity to an amino acid sequence of SEQ ID NO: 262, a CDR L2 having an amino acid sequence 100% identical to the amino acid sequence of GAS, and a CDR L3 having an amino acid sequence 100% identical to the amino acid sequence of SEQ ID NO: 263. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 257 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 261. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 257 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 261. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 257 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 261. In some embodiments, the antibody comprises a variable heavy chain (VH) region having an amino acid sequence with at least 95% (e.g., at least 95%, 96%, 97%, 98%, 99%, or more) identity to SEQ ID NO: 257 and a variable light chain (VL) region having an amino acid sequence with at least 95% (e.g., at least 95%, 96%, 97%, 98%, 99%, or more) identity to SEQ ID NO: 261. In some embodiments, the antibody comprises a variable heavy chain (VH) region having an amino acid sequence 100% identical with the amino acid sequence of SEQ ID NO: 257 and a variable light chain (VL) region having an amino acid sequence 100% identical with the amino acid sequence of SEQ ID NO: 261. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 257 and comprising therein a complementarity determining region (CDR) H1 having an amino acid sequence 100% identical to the amino acid sequence of SEQ ID NO: 258, a CDR H2 having an amino acid sequence 100% identical to the amino acid sequence of SEQ ID NO: 259, and a CDR H3 having an amino acid sequence 100% identical to the amino acid sequence of SEQ ID NO: 260. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 261 and comprising therein a complementarity determining region (CDR) L1 having 100% sequence identity to an amino acid sequence of SEQ ID NO: 262, a CDR L2 having an amino acid sequence 100% identical to the amino acid sequence of GAS, and a CDR L3 having an amino acid sequence 100% identical to the amino acid sequence of SEQ ID NO: 263. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 257 and comprising therein a complementarity determining region (CDR) H1 having an amino acid sequence 100% identical to the amino acid sequence of SEQ ID NO: 258, a CDR H2 having an amino acid sequence 100% identical to the amino acid sequence of SEQ ID NO: 259, and a CDR H3 having an amino acid sequence 100% identical to the amino acid sequence of SEQ ID NO: 260; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 261 and comprising therein a complementarity determining region (CDR) L1 having 100% sequence identity to an amino acid sequence of SEQ ID NO: 262, a CDR L2 having an amino acid sequence 100% identical to the amino acid sequence of GAS, and a CDR L3 having an amino acid sequence 100% identical to the amino acid sequence of SEQ ID NO: 263.

In some embodiments, the antibody comprises a complementarity determining region (CDR) H1 having an amino acid sequence 100% identical to the amino acid sequence of SEQ ID NO: 265, a CDR H2 having an amino acid sequence 100% identical to the amino acid sequence of SEQ ID NO: 266, and a CDR H3 having an amino acid sequence 100% identical to the amino acid sequence of SEQ ID NO: 267. In some embodiments, the antibody comprises a complementarity determining region (CDR) L1 having 100% sequence identity to an amino acid sequence of SEQ ID NO: 269, a CDR L2 having an amino acid sequence 100% identical to the amino acid sequence of DAV, and a CDR L3 having an amino acid sequence 100% identical to the amino acid sequence of SEQ ID NO: 270. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 264. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 268. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 264. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 268. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 264. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 268. In some embodiments, the antibody comprises a variable heavy chain (VH) region having an amino acid sequence with at least 95% (e.g., at least 95%, 96%, 97%, 98%, 99%, or more) identity to SEQ ID NO: 264. In some embodiments, the antibody comprises a variable light chain (VL) region having an amino acid sequence with at least 95% (e.g., at least 95%, 96%, 97%, 98%, 99%, or more) identity to SEQ ID NO: 268. In some embodiments, the antibody comprises a variable heavy chain (VH) region having an amino acid sequence 100% identical with the amino acid sequence of SEQ ID NO: 264. In some embodiments, the antibody comprises a variable light chain (VL) region having an amino acid sequence 100% identical with the amino acid sequence of SEQ ID NO: 268. In some embodiments, the antibody comprises: (a) a complementarity determining region (CDR) H1 having an amino acid sequence 100% identical to the amino acid sequence of SEQ ID NO: 265, a CDR H2 having an amino acid sequence 100% identical to the amino acid sequence of SEQ ID NO: 266, and a CDR H3 having an amino acid sequence 100% identical to the amino acid sequence of SEQ ID NO: 267; and (b) a complementarity determining region (CDR) L1 having 100% sequence identity to an amino acid sequence of SEQ ID NO: 269, a CDR L2 having an amino acid sequence 100% identical to the amino acid sequence of DAV, and a CDR L3 having an amino acid sequence 100% identical to the amino acid sequence of SEQ ID NO: 270. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 264 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 268. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 264 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 268. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 264 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 268. In some embodiments, the antibody comprises a variable heavy chain (VH) region having an amino acid sequence with at least 95% (e.g., at least 95%, 96%, 97%, 98%, 99%, or more) identity to SEQ ID NO: 264 and a variable light chain (VL) region having an amino acid sequence with at least 95% (e.g., at least 95%, 96%, 97%, 98%, 99%, or more) identity to SEQ ID NO: 268. In some embodiments, the antibody comprises a variable heavy chain (VH) region having an amino acid sequence 100% identical with the amino acid sequence of SEQ ID NO: 264 and a variable light chain (VL) region having an amino acid sequence 100% identical with the amino acid sequence of SEQ ID NO: 268. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 264 and comprising therein a complementarity determining region (CDR) H1 having an amino acid sequence 100% identical to the amino acid sequence of SEQ ID NO: 265, a CDR H2 having an amino acid sequence 100% identical to the amino acid sequence of SEQ ID NO: 266, and a CDR H3 having an amino acid sequence 100% identical to the amino acid sequence of SEQ ID NO: 267. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 268 and comprising therein a complementarity determining region (CDR) L1 having 100% sequence identity to an amino acid sequence of SEQ ID NO: 269, a CDR L2 having an amino acid sequence 100% identical to the amino acid sequence of DAV, and a CDR L3 having an amino acid sequence 100% identical to the amino acid sequence of SEQ ID NO: 270. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 264 and comprising therein a complementarity determining region (CDR) H1 having an amino acid sequence 100% identical to the amino acid sequence of SEQ ID NO: 265, a CDR H2 having an amino acid sequence 100% identical to the amino acid sequence of SEQ ID NO: 266, and a CDR H3 having an amino acid sequence 100% identical to the amino acid sequence of SEQ ID NO: 267; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 268 and comprising therein a complementarity determining region (CDR) L1 having 100% sequence identity to an amino acid sequence of SEQ ID NO: 269, a CDR L2 having an amino acid sequence 100% identical to the amino acid sequence of DAV, and a CDR L3 having an amino acid sequence 100% identical to the amino acid sequence of SEQ ID NO: 270.

In some embodiments, the antibody comprises a complementarity determining region (CDR) H1 having an amino acid sequence 100% identical to the amino acid sequence of SEQ ID NO: 272, a CDR H2 having an amino acid sequence 100% identical to the amino acid sequence of SEQ ID NO: 273, and a CDR H3 having an amino acid sequence 100% identical to the amino acid sequence of SEQ ID NO: 274. In some embodiments, the antibody comprises a complementarity determining region (CDR) L1 having 100% sequence identity to an amino acid sequence of SEQ ID NO: 276, a CDR L2 having an amino acid sequence 100% identical to the amino acid sequence of DAS, and a CDR L3 having an amino acid sequence 100% identical to the amino acid sequence of SEQ ID NO: 277. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 271. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 275. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 271. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 275. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 271. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 275. In some embodiments, the antibody comprises a variable heavy chain (VH) region having an amino acid sequence with at least 95% (e.g., at least 95%, 96%, 97%, 98%, 99%, or more) identity to SEQ ID NO: 271. In some embodiments, the antibody comprises a variable light chain (VL) region having an amino acid sequence with at least 95% (e.g., at least 95%, 96%, 97%, 98%, 99%, or more) identity to SEQ ID NO: 275. In some embodiments, the antibody comprises a variable heavy chain (VH) region having an amino acid sequence 100% identical with the amino acid sequence of SEQ ID NO: 271. In some embodiments, the antibody comprises a variable light chain (VL) region having an amino acid sequence 100% identical with the amino acid sequence of SEQ ID NO: 275. In some embodiments, the antibody comprises: (a) a complementarity determining region (CDR) H1 having an amino acid sequence 100% identical to the amino acid sequence of SEQ ID NO: 272, a CDR H2 having an amino acid sequence 100% identical to the amino acid sequence of SEQ ID NO: 273, and a CDR H3 having an amino acid sequence 100% identical to the amino acid sequence of SEQ ID NO: 274; and (b) a complementarity determining region (CDR) L1 having 100% sequence identity to an amino acid sequence of SEQ ID NO: 276, a CDR L2 having an amino acid sequence 100% identical to the amino acid sequence of DAS, and a CDR L3 having an amino acid sequence 100% identical to the amino acid sequence of SEQ ID NO: 277. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 271 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 275. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 271 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 275. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 271 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 275. In some embodiments, the antibody comprises a variable heavy chain (VH) region having an amino acid sequence with at least 95% (e.g., at least 95%, 96%, 97%, 98%, 99%, or more) identity to SEQ ID NO: 271 and a variable light chain (VL) region having an amino acid sequence with at least 95% (e.g., at least 95%, 96%, 97%, 98%, 99%, or more) identity to SEQ ID NO: 275. In some embodiments, the antibody comprises a variable heavy chain (VH) region having an amino acid sequence 100% identical with the amino acid sequence of SEQ ID NO: 271 and a variable light chain (VL) region having an amino acid sequence 100% identical with the amino acid sequence of SEQ ID NO: 275. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 271 and comprising therein a complementarity determining region (CDR) H1 having an amino acid sequence 100% identical to the amino acid sequence of SEQ ID NO: 272, a CDR H2 having an amino acid sequence 100% identical to the amino acid sequence of SEQ ID NO: 273, and a CDR H3 having an amino acid sequence 100% identical to the amino acid sequence of SEQ ID NO: 274. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 275 and comprising therein a complementarity determining region (CDR) L1 having 100% sequence identity to an amino acid sequence of SEQ ID NO: 276, a CDR L2 having an amino acid sequence 100% identical to the amino acid sequence of DAS, and a CDR L3 having an amino acid sequence 100% identical to the amino acid sequence of SEQ ID NO: 277. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 271 and comprising therein a complementarity determining region (CDR) H1 having an amino acid sequence 100% identical to the amino acid sequence of SEQ ID NO: 272, a CDR H2 having an amino acid sequence 100% identical to the amino acid sequence of SEQ ID NO: 273, and a CDR H3 having an amino acid sequence 100% identical to the amino acid sequence of SEQ ID NO: 274; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 275 and comprising therein a complementarity determining region (CDR) L1 having 100% sequence identity to an amino acid sequence of SEQ ID NO: 276, a CDR L2 having an amino acid sequence 100% identical to the amino acid sequence of DAS, and a CDR L3 having an amino acid sequence 100% identical to the amino acid sequence of SEQ ID NO: 277.

In some embodiments, the antibody comprises a complementarity determining region (CDR) H1 having an amino acid sequence 100% identical to the amino acid sequence of SEQ ID NO: 279, a CDR H2 having an amino acid sequence 100% identical to the amino acid sequence of SEQ ID NO: 280, and a CDR H3 having an amino acid sequence 100% identical to the amino acid sequence of SEQ ID NO: 281. In some embodiments, the antibody comprises a complementarity determining region (CDR) L1 having 100% sequence identity to an amino acid sequence of SEQ ID NO: 283, a CDR L2 having an amino acid sequence 100% identical to the amino acid sequence of DAS, and a CDR L3 having an amino acid sequence 100% identical to the amino acid sequence of SEQ ID NO: 284. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 278. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 282. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 278. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 282. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 278. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 282. In some embodiments, the antibody comprises a variable heavy chain (VH) region having an amino acid sequence with at least 95% (e.g., at least 95%, 96%, 97%, 98%, 99%, or more) identity to SEQ ID NO: 278. In some embodiments, the antibody comprises a variable light chain (VL) region having an amino acid sequence with at least 95% (e.g., at least 95%, 96%, 97%, 98%, 99%, or more) identity to SEQ ID NO: 282. In some embodiments, the antibody comprises a variable heavy chain (VH) region having an amino acid sequence 100% identical with the amino acid sequence of SEQ ID NO: 278. In some embodiments, the antibody comprises a variable light chain (VL) region having an amino acid sequence 100% identical with the amino acid sequence of SEQ ID NO: 282. In some embodiments, the antibody comprises: (a) a complementarity determining region (CDR) H1 having an amino acid sequence 100% identical to the amino acid sequence of SEQ ID NO: 279, a CDR H2 having an amino acid sequence 100% identical to the amino acid sequence of SEQ ID NO: 280, and a CDR H3 having an amino acid sequence 100% identical to the amino acid sequence of SEQ ID NO: 281; and (b) a complementarity determining region (CDR) L1 having 100% sequence identity to an amino acid sequence of SEQ ID NO: 283, a CDR L2 having an amino acid sequence 100% identical to the amino acid sequence of DAS, and a CDR L3 having an amino acid sequence 100% identical to the amino acid sequence of SEQ ID NO: 284. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 278 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 282. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 278 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 282. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 278 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 282. In some embodiments, the antibody comprises a variable heavy chain (VH) region having an amino acid sequence with at least 95% (e.g., at least 95%, 96%, 97%, 98%, 99%, or more) identity to SEQ ID NO: 278 and a variable light chain (VL) region having an amino acid sequence with at least 95% (e.g., at least 95%, 96%, 97%, 98%, 99%, or more) identity to SEQ ID NO: 282. In some embodiments, the antibody comprises a variable heavy chain (VH) region having an amino acid sequence 100% identical with the amino acid sequence of SEQ ID NO: 278 and a variable light chain (VL) region having an amino acid sequence 100% identical with the amino acid sequence of SEQ ID NO: 282. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 278 and comprising therein a complementarity determining region (CDR) H1 having an amino acid sequence 100% identical to the amino acid sequence of SEQ ID NO: 279, a CDR H2 having an amino acid sequence 100% identical to the amino acid sequence of SEQ ID NO: 280, and a CDR H3 having an amino acid sequence 100% identical to the amino acid sequence of SEQ ID NO: 281. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 282 and comprising therein a complementarity determining region (CDR) L1 having 100% sequence identity to an amino acid sequence of SEQ ID NO: 283, a CDR L2 having an amino acid sequence 100% identical to the amino acid sequence of DAS, and a CDR L3 having an amino acid sequence 100% identical to the amino acid sequence of SEQ ID NO: 284. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 278 and comprising therein a complementarity determining region (CDR) H1 having an amino acid sequence 100% identical to the amino acid sequence of SEQ ID NO: 279, a CDR H2 having an amino acid sequence 100% identical to the amino acid sequence of SEQ ID NO: 280, and a CDR H3 having an amino acid sequence 100% identical to the amino acid sequence of SEQ ID NO: 281; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 282 and comprising therein a complementarity determining region (CDR) L1 having 100% sequence identity to an amino acid sequence of SEQ ID NO: 283, a CDR L2 having an amino acid sequence 100% identical to the amino acid sequence of DAS, and a CDR L3 having an amino acid sequence 100% identical to the amino acid sequence of SEQ ID NO: 284.

In some embodiments, the antibody comprises a complementarity determining region (CDR) H1 having an amino acid sequence 100% identical to the amino acid sequence of SEQ ID NO: 286, a CDR H2 having an amino acid sequence 100% identical to the amino acid sequence of SEQ ID NO: 287, and a CDR H3 having an amino acid sequence 100% identical to the amino acid sequence of SEQ ID NO: 288. In some embodiments, the antibody comprises a complementarity determining region (CDR) L1 having 100% sequence identity to an amino acid sequence of SEQ ID NO: 290, a CDR L2 having an amino acid sequence 100% identical to the amino acid sequence of GAS, and a CDR L3 having an amino acid sequence 100% identical to the amino acid sequence of SEQ ID NO: 291. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 285. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 289. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 285. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 289. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 285. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 289. In some embodiments, the antibody comprises a variable heavy chain (VH) region having an amino acid sequence with at least 95% (e.g., at least 95%, 96%, 97%, 98%, 99%, or more) identity to SEQ ID NO: 285. In some embodiments, the antibody comprises a variable light chain (VL) region having an amino acid sequence with at least 95% (e.g., at least 95%, 96%, 97%, 98%, 99%, or more) identity to SEQ ID NO: 289. In some embodiments, the antibody comprises a variable heavy chain (VH) region having an amino acid sequence 100% identical with the amino acid sequence of SEQ ID NO: 285. In some embodiments, the antibody comprises a variable light chain (VL) region having an amino acid sequence 100% identical with the amino acid sequence of SEQ ID NO: 289. In some embodiments, the antibody comprises: (a) a complementarity determining region (CDR) H1 having an amino acid sequence 100% identical to the amino acid sequence of SEQ ID NO: 286, a CDR H2 having an amino acid sequence 100% identical to the amino acid sequence of SEQ ID NO: 287, and a CDR H3 having an amino acid sequence 100% identical to the amino acid sequence of SEQ ID NO: 288; and (b) a complementarity determining region (CDR) L1 having 100% sequence identity to an amino acid sequence of SEQ ID NO: 290, a CDR L2 having an amino acid sequence 100% identical to the amino acid sequence of GAS, and a CDR L3 having an amino acid sequence 100% identical to the amino acid sequence of SEQ ID NO: 291. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 285 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 289. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 285 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 289. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 285 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 289. In some embodiments, the antibody comprises a variable heavy chain (VH) region having an amino acid sequence with at least 95% (e.g., at least 95%, 96%, 97%, 98%, 99%, or more) identity to SEQ ID NO: 285 and a variable light chain (VL) region having an amino acid sequence with at least 95% (e.g., at least 95%, 96%, 97%, 98%, 99%, or more) identity to SEQ ID NO: 289. In some embodiments, the antibody comprises a variable heavy chain (VH) region having an amino acid sequence 100% identical with the amino acid sequence of SEQ ID NO: 285 and a variable light chain (VL) region having an amino acid sequence 100% identical with the amino acid sequence of SEQ ID NO: 289. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 285 and comprising therein a complementarity determining region (CDR) H1 having an amino acid sequence 100% identical to the amino acid sequence of SEQ ID NO: 286, a CDR H2 having an amino acid sequence 100% identical to the amino acid sequence of SEQ ID NO: 287, and a CDR H3 having an amino acid sequence 100% identical to the amino acid sequence of SEQ ID NO: 288. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 289 and comprising therein a complementarity determining region (CDR) L1 having 100% sequence identity to an amino acid sequence of SEQ ID NO: 290, a CDR L2 having an amino acid sequence 100% identical to the amino acid sequence of GAS, and a CDR L3 having an amino acid sequence 100% identical to the amino acid sequence of SEQ ID NO: 291. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 285 and comprising therein a complementarity determining region (CDR) H1 having an amino acid sequence 100% identical to the amino acid sequence of SEQ ID NO: 286, a CDR H2 having an amino acid sequence 100% identical to the amino acid sequence of SEQ ID NO: 287, and a CDR H3 having an amino acid sequence 100% identical to the amino acid sequence of SEQ ID NO: 288; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 289 and comprising therein a complementarity determining region (CDR) L1 having 100% sequence identity to an amino acid sequence of SEQ ID NO: 290, a CDR L2 having an amino acid sequence 100% identical to the amino acid sequence of GAS, and a CDR L3 having an amino acid sequence 100% identical to the amino acid sequence of SEQ ID NO: 291.

In some embodiments, the antibody comprises a complementarity determining region (CDR) H1 having an amino acid sequence 100% identical to the amino acid sequence of SEQ ID NO: 293, a CDR H2 having an amino acid sequence 100% identical to the amino acid sequence of SEQ ID NO: 294, and a CDR H3 having an amino acid sequence 100% identical to the amino acid sequence of SEQ ID NO: 295. In some embodiments, the antibody comprises a complementarity determining region (CDR) L1 having 100% sequence identity to an amino acid sequence of SEQ ID NO: 297, a CDR L2 having an amino acid sequence 100% identical to the amino acid sequence of KAS, and a CDR L3 having an amino acid sequence 100% identical to the amino acid sequence of SEQ ID NO: 298. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 292. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 296. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 292. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 296. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 292. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 296. In some embodiments, the antibody comprises a variable heavy chain (VH) region having an amino acid sequence with at least 95% (e.g., at least 95%, 96%, 97%, 98%, 99%, or more) identity to SEQ ID NO: 292. In some embodiments, the antibody comprises a variable light chain (VL) region having an amino acid sequence with at least 95% (e.g., at least 95%, 96%, 97%, 98%, 99%, or more) identity to SEQ ID NO: 296. In some embodiments, the antibody comprises a variable heavy chain (VH) region having an amino acid sequence 100% identical with the amino acid sequence of SEQ ID NO: 292. In some embodiments, the antibody comprises a variable light chain (VL) region having an amino acid sequence 100% identical with the amino acid sequence of SEQ ID NO: 296. In some embodiments, the antibody comprises: (a) a complementarity determining region (CDR) H1 having an amino acid sequence 100% identical to the amino acid sequence of SEQ ID NO: 293, a CDR H2 having an amino acid sequence 100% identical to the amino acid sequence of SEQ ID NO: 294, and a CDR H3 having an amino acid sequence 100% identical to the amino acid sequence of SEQ ID NO: 295; and (b) a complementarity determining region (CDR) L1 having 100% sequence identity to an amino acid sequence of SEQ ID NO: 297, a CDR L2 having an amino acid sequence 100% identical to the amino acid sequence of KAS, and a CDR L3 having an amino acid sequence 100% identical to the amino acid sequence of SEQ ID NO: 298. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 292 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 296. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 292 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 296. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 292 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 296. In some embodiments, the antibody comprises a variable heavy chain (VH) region having an amino acid sequence with at least 95% (e.g., at least 95%, 96%, 97%, 98%, 99%, or more) identity to SEQ ID NO: 292 and a variable light chain (VL) region having an amino acid sequence with at least 95% (e.g., at least 95%, 96%, 97%, 98%, 99%, or more) identity to SEQ ID NO: 296. In some embodiments, the antibody comprises a variable heavy chain (VH) region having an amino acid sequence 100% identical with the amino acid sequence of SEQ ID NO: 292 and a variable light chain (VL) region having an amino acid sequence 100% identical with the amino acid sequence of SEQ ID NO: 296. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 292 and comprising therein a complementarity determining region (CDR) H1 having an amino acid sequence 100% identical to the amino acid sequence of SEQ ID NO: 293, a CDR H2 having an amino acid sequence 100% identical to the amino acid sequence of SEQ ID NO: 294, and a CDR H3 having an amino acid sequence 100% identical to the amino acid sequence of SEQ ID NO: 295. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 296 and comprising therein a complementarity determining region (CDR) L1 having 100% sequence identity to an amino acid sequence of SEQ ID NO: 297, a CDR L2 having an amino acid sequence 100% identical to the amino acid sequence of KAS, and a CDR L3 having an amino acid sequence 100% identical to the amino acid sequence of SEQ ID NO: 298. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 292 and comprising therein a complementarity determining region (CDR) H1 having an amino acid sequence 100% identical to the amino acid sequence of SEQ ID NO: 293, a CDR H2 having an amino acid sequence 100% identical to the amino acid sequence of SEQ ID NO: 294, and a CDR H3 having an amino acid sequence 100% identical to the amino acid sequence of SEQ ID NO: 295; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 296 and comprising therein a complementarity determining region (CDR) L1 having 100% sequence identity to an amino acid sequence of SEQ ID NO: 297, a CDR L2 having an amino acid sequence 100% identical to the amino acid sequence of KAS, and a CDR L3 having an amino acid sequence 100% identical to the amino acid sequence of SEQ ID NO: 298.

In some embodiments, the antibody comprises a complementarity determining region (CDR) H1 having an amino acid sequence 100% identical to the amino acid sequence of SEQ ID NO: 300, a CDR H2 having an amino acid sequence 100% identical to the amino acid sequence of SEQ ID NO: 301, and a CDR H3 having an amino acid sequence 100% identical to the amino acid sequence of SEQ ID NO: 302. In some embodiments, the antibody comprises a complementarity determining region (CDR) L1 having 100% sequence identity to an amino acid sequence of SEQ ID NO: 304, a CDR L2 having an amino acid sequence 100% identical to the amino acid sequence of AAS, and a CDR L3 having an amino acid sequence 100% identical to the amino acid sequence of SEQ ID NO: 305. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 299. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 303. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 299. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 303. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 299. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 303. In some embodiments, the antibody comprises a variable heavy chain (VH) region having an amino acid sequence with at least 95% (e.g., at least 95%, 96%, 97%, 98%, 99%, or more) identity to SEQ ID NO: 299. In some embodiments, the antibody comprises a variable light chain (VL) region having an amino acid sequence with at least 95% (e.g., at least 95%, 96%, 97%, 98%, 99%, or more) identity to SEQ ID NO: 303. In some embodiments, the antibody comprises a variable heavy chain (VH) region having an amino acid sequence 100% identical with the amino acid sequence of SEQ ID NO: 299. In some embodiments, the antibody comprises a variable light chain (VL) region having an amino acid sequence 100% identical with the amino acid sequence of SEQ ID NO: 303. In some embodiments, the antibody comprises: (a) a complementarity determining region (CDR) H1 having an amino acid sequence 100% identical to the amino acid sequence of SEQ ID NO: 300, a CDR H2 having an amino acid sequence 100% identical to the amino acid sequence of SEQ ID NO: 301, and a CDR H3 having an amino acid sequence 100% identical to the amino acid sequence of SEQ ID NO: 302; and (b) a complementarity determining region (CDR) L1 having 100% sequence identity to an amino acid sequence of SEQ ID NO: 304, a CDR L2 having an amino acid sequence 100% identical to the amino acid sequence of AAS, and a CDR L3 having an amino acid sequence 100% identical to the amino acid sequence of SEQ ID NO: 305. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 299 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 303. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 299 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 303. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 299 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 303. In some embodiments, the antibody comprises a variable heavy chain (VH) region having an amino acid sequence with at least 95% (e.g., at least 95%, 96%, 97%, 98%, 99%, or more) identity to SEQ ID NO: 299 and a variable light chain (VL) region having an amino acid sequence with at least 95% (e.g., at least 95%, 96%, 97%, 98%, 99%, or more) identity to SEQ ID NO: 303. In some embodiments, the antibody comprises a variable heavy chain (VH) region having an amino acid sequence 100% identical with the amino acid sequence of SEQ ID NO: 299 and a variable light chain (VL) region having an amino acid sequence 100% identical with the amino acid sequence of SEQ ID NO: 303. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 299 and comprising therein a complementarity determining region (CDR) H1 having an amino acid sequence 100% identical to the amino acid sequence of SEQ ID NO: 300, a CDR H2 having an amino acid sequence 100% identical to the amino acid sequence of SEQ ID NO: 301, and a CDR H3 having an amino acid sequence 100% identical to the amino acid sequence of SEQ ID NO: 302. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 303 and comprising therein a complementarity determining region (CDR) L1 having 100% sequence identity to an amino acid sequence of SEQ ID NO: 304, a CDR L2 having an amino acid sequence 100% identical to the amino acid sequence of AAS, and a CDR L3 having an amino acid sequence 100% identical to the amino acid sequence of SEQ ID NO: 305. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 299 and comprising therein a complementarity determining region (CDR) H1 having an amino acid sequence 100% identical to the amino acid sequence of SEQ ID NO: 300, a CDR H2 having an amino acid sequence 100% identical to the amino acid sequence of SEQ ID NO: 301, and a CDR H3 having an amino acid sequence 100% identical to the amino acid sequence of SEQ ID NO: 302; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 303 and comprising therein a complementarity determining region (CDR) L1 having 100% sequence identity to an amino acid sequence of SEQ ID NO: 304, a CDR L2 having an amino acid sequence 100% identical to the amino acid sequence of AAS, and a CDR L3 having an amino acid sequence 100% identical to the amino acid sequence of SEQ ID NO: 305.

In some embodiments, the antibody comprises a complementarity determining region (CDR) H1 having an amino acid sequence 100% identical to the amino acid sequence of SEQ ID NO: 307, a CDR H2 having an amino acid sequence 100% identical to the amino acid sequence of SEQ ID NO: 308, and a CDR H3 having an amino acid sequence 100% identical to the amino acid sequence of SEQ ID NO: 309. In some embodiments, the antibody comprises a complementarity determining region (CDR) L1 having 100% sequence identity to an amino acid sequence of SEQ ID NO: 311, a CDR L2 having an amino acid sequence 100% identical to the amino acid sequence of DTS, and a CDR L3 having an amino acid sequence 100% identical to the amino acid sequence of SEQ ID NO: 312. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 306. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 310. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 306. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 310. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 306. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 310. In some embodiments, the antibody comprises a variable heavy chain (VH) region having an amino acid sequence with at least 95% (e.g., at least 95%, 96%, 97%, 98%, 99%, or more) identity to SEQ ID NO: 306. In some embodiments, the antibody comprises a variable light chain (VL) region having an amino acid sequence with at least 95% (e.g., at least 95%, 96%, 97%, 98%, 99%, or more) identity to SEQ ID NO: 310. In some embodiments, the antibody comprises a variable heavy chain (VH) region having an amino acid sequence 100% identical with the amino acid sequence of SEQ ID NO: 306. In some embodiments, the antibody comprises a variable light chain (VL) region having an amino acid sequence 100% identical with the amino acid sequence of SEQ ID NO: 310. In some embodiments, the antibody comprises: (a) a complementarity determining region (CDR) H1 having an amino acid sequence 100% identical to the amino acid sequence of SEQ ID NO: 307, a CDR H2 having an amino acid sequence 100% identical to the amino acid sequence of SEQ ID NO: 308, and a CDR H3 having an amino acid sequence 100% identical to the amino acid sequence of SEQ ID NO: 309; and (b) a complementarity determining region (CDR) L1 having 100% sequence identity to an amino acid sequence of SEQ ID NO: 311, a CDR L2 having an amino acid sequence 100% identical to the amino acid sequence of DTS, and a CDR L3 having an amino acid sequence 100% identical to the amino acid sequence of SEQ ID NO: 312. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 306 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 310. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 306 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 310. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 306 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 310. In some embodiments, the antibody comprises a variable heavy chain (VH) region having an amino acid sequence with at least 95% (e.g., at least 95%, 96%, 97%, 98%, 99%, or more) identity to SEQ ID NO: 306 and a variable light chain (VL) region having an amino acid sequence with at least 95% (e.g., at least 95%, 96%, 97%, 98%, 99%, or more) identity to SEQ ID NO: 310. In some embodiments, the antibody comprises a variable heavy chain (VH) region having an amino acid sequence 100% identical with the amino acid sequence of SEQ ID NO: 306 and a variable light chain (VL) region having an amino acid sequence 100% identical with the amino acid sequence of SEQ ID NO: 310. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 306 and comprising therein a complementarity determining region (CDR) H1 having an amino acid sequence 100% identical to the amino acid sequence of SEQ ID NO: 307, a CDR H2 having an amino acid sequence 100% identical to the amino acid sequence of SEQ ID NO: 308, and a CDR H3 having an amino acid sequence 100% identical to the amino acid sequence of SEQ ID NO: 309. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 310 and comprising therein a complementarity determining region (CDR) L1 having 100% sequence identity to an amino acid sequence of SEQ ID NO: 311, a CDR L2 having an amino acid sequence 100% identical to the amino acid sequence of DTS, and a CDR L3 having an amino acid sequence 100% identical to the amino acid sequence of SEQ ID NO: 312. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 306 and comprising therein a complementarity determining region (CDR) H1 having an amino acid sequence 100% identical to the amino acid sequence of SEQ ID NO: 307, a CDR H2 having an amino acid sequence 100% identical to the amino acid sequence of SEQ ID NO: 308, and a CDR H3 having an amino acid sequence 100% identical to the amino acid sequence of SEQ ID NO: 309; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 310 and comprising therein a complementarity determining region (CDR) L1 having 100% sequence identity to an amino acid sequence of SEQ ID NO: 311, a CDR L2 having an amino acid sequence 100% identical to the amino acid sequence of DTS, and a CDR L3 having an amino acid sequence 100% identical to the amino acid sequence of SEQ ID NO: 312. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 461. In some embodiments, the antibody comprises a second heavy chain region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 481. In some embodiments, the antibody comprises a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 471. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 461, a second heavy chain region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 481, and a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 471.

In some embodiments, the antibody comprises a complementarity determining region (CDR) H1 having an amino acid sequence 100% identical to the amino acid sequence of SEQ ID NO: 314, a CDR H2 having an amino acid sequence 100% identical to the amino acid sequence of SEQ ID NO: 315, and a CDR H3 having an amino acid sequence 100% identical to the amino acid sequence of SEQ ID NO: 316. In some embodiments, the antibody comprises a complementarity determining region (CDR) L1 having 100% sequence identity to an amino acid sequence of SEQ ID NO: 318, a CDR L2 having an amino acid sequence 100% identical to the amino acid sequence of DAS, and a CDR L3 having an amino acid sequence 100% identical to the amino acid sequence of SEQ ID NO: 319. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 313. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 317. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 313. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 317. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 313. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 317. In some embodiments, the antibody comprises a variable heavy chain (VH) region having an amino acid sequence with at least 95% (e.g., at least 95%, 96%, 97%, 98%, 99%, or more) identity to SEQ ID NO: 313. In some embodiments, the antibody comprises a variable light chain (VL) region having an amino acid sequence with at least 95% (e.g., at least 95%, 96%, 97%, 98%, 99%, or more) identity to SEQ ID NO: 317. In some embodiments, the antibody comprises a variable heavy chain (VH) region having an amino acid sequence 100% identical with the amino acid sequence of SEQ ID NO: 313. In some embodiments, the antibody comprises a variable light chain (VL) region having an amino acid sequence 100% identical with the amino acid sequence of SEQ ID NO: 317. In some embodiments, the antibody comprises: (a) a complementarity determining region (CDR) H1 having an amino acid sequence 100% identical to the amino acid sequence of SEQ ID NO: 314, a CDR H2 having an amino acid sequence 100% identical to the amino acid sequence of SEQ ID NO: 315, and a CDR H3 having an amino acid sequence 100% identical to the amino acid sequence of SEQ ID NO: 316; and (b) a complementarity determining region (CDR) L1 having 100% sequence identity to an amino acid sequence of SEQ ID NO: 318, a CDR L2 having an amino acid sequence 100% identical to the amino acid sequence of DAS, and a CDR L3 having an amino acid sequence 100% identical to the amino acid sequence of SEQ ID NO: 319. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 313 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 317. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 313 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 317. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 313 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 317. In some embodiments, the antibody comprises a variable heavy chain (VH) region having an amino acid sequence with at least 95% (e.g., at least 95%, 96%, 97%, 98%, 99%, or more) identity to SEQ ID NO: 313 and a variable light chain (VL) region having an amino acid sequence with at least 95% (e.g., at least 95%, 96%, 97%, 98%, 99%, or more) identity to SEQ ID NO: 317. In some embodiments, the antibody comprises a variable heavy chain (VH) region having an amino acid sequence 100% identical with the amino acid sequence of SEQ ID NO: 313 and a variable light chain (VL) region having an amino acid sequence 100% identical with the amino acid sequence of SEQ ID NO: 317. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 313 and comprising therein a complementarity determining region (CDR) H1 having an amino acid sequence 100% identical to the amino acid sequence of SEQ ID NO: 314, a CDR H2 having an amino acid sequence 100% identical to the amino acid sequence of SEQ ID NO: 315, and a CDR H3 having an amino acid sequence 100% identical to the amino acid sequence of SEQ ID NO: 316. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 317 and comprising therein a complementarity determining region (CDR) L1 having 100% sequence identity to an amino acid sequence of SEQ ID NO: 318, a CDR L2 having an amino acid sequence 100% identical to the amino acid sequence of DAS, and a CDR L3 having an amino acid sequence 100% identical to the amino acid sequence of SEQ ID NO: 319. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 313 and comprising therein a complementarity determining region (CDR) H1 having an amino acid sequence 100% identical to the amino acid sequence of SEQ ID NO: 314, a CDR H2 having an amino acid sequence 100% identical to the amino acid sequence of SEQ ID NO: 315, and a CDR H3 having an amino acid sequence 100% identical to the amino acid sequence of SEQ ID NO: 316; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 317 and comprising therein a complementarity determining region (CDR) L1 having 100% sequence identity to an amino acid sequence of SEQ ID NO: 318, a CDR L2 having an amino acid sequence 100% identical to the amino acid sequence of DAS, and a CDR L3 having an amino acid sequence 100% identical to the amino acid sequence of SEQ ID NO: 319.

In some embodiments, the antibody comprises a complementarity determining region (CDR) H1 having an amino acid sequence 100% identical to the amino acid sequence of SEQ ID NO: 321, a CDR H2 having an amino acid sequence 100% identical to the amino acid sequence of SEQ ID NO: 322, and a CDR H3 having an amino acid sequence 100% identical to the amino acid sequence of SEQ ID NO: 323. In some embodiments, the antibody comprises a complementarity determining region (CDR) L1 having 100% sequence identity to an amino acid sequence of SEQ ID NO: 325, a CDR L2 having an amino acid sequence 100% identical to the amino acid sequence of STS, and a CDR L3 having an amino acid sequence 100% identical to the amino acid sequence of SEQ ID NO: 326. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 320. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 324. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 320. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 324. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 320. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 324. In some embodiments, the antibody comprises a variable heavy chain (VH) region having an amino acid sequence with at least 95% (e.g., at least 95%, 96%, 97%, 98%, 99%, or more) identity to SEQ ID NO: 320. In some embodiments, the antibody comprises a variable light chain (VL) region having an amino acid sequence with at least 95% (e.g., at least 95%, 96%, 97%, 98%, 99%, or more) identity to SEQ ID NO: 324. In some embodiments, the antibody comprises a variable heavy chain (VH) region having an amino acid sequence 100% identical with the amino acid sequence of SEQ ID NO: 320. In some embodiments, the antibody comprises a variable light chain (VL) region having an amino acid sequence 100% identical with the amino acid sequence of SEQ ID NO: 324. In some embodiments, the antibody comprises: (a) a complementarity determining region (CDR) H1 having an amino acid sequence 100% identical to the amino acid sequence of SEQ ID NO: 321, a CDR H2 having an amino acid sequence 100% identical to the amino acid sequence of SEQ ID NO: 322, and a CDR H3 having an amino acid sequence 100% identical to the amino acid sequence of SEQ ID NO: 323; and (b) a complementarity determining region (CDR) L1 having 100% sequence identity to an amino acid sequence of SEQ ID NO: 325, a CDR L2 having an amino acid sequence 100% identical to the amino acid sequence of STS, and a CDR L3 having an amino acid sequence 100% identical to the amino acid sequence of SEQ ID NO: 326. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 320 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 324. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 320 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 324. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 320 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 324. In some embodiments, the antibody comprises a variable heavy chain (VH) region having an amino acid sequence with at least 95% (e.g., at least 95%, 96%, 97%, 98%, 99%, or more) identity to SEQ ID NO: 320 and a variable light chain (VL) region having an amino acid sequence with at least 95% (e.g., at least 95%, 96%, 97%, 98%, 99%, or more) identity to SEQ ID NO: 324. In some embodiments, the antibody comprises a variable heavy chain (VH) region having an amino acid sequence 100% identical with the amino acid sequence of SEQ ID NO: 320 and a variable light chain (VL) region having an amino acid sequence 100% identical with the amino acid sequence of SEQ ID NO: 324. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 320 and comprising therein a complementarity determining region (CDR) H1 having an amino acid sequence 100% identical to the amino acid sequence of SEQ ID NO: 321, a CDR H2 having an amino acid sequence 100% identical to the amino acid sequence of SEQ ID NO: 322, and a CDR H3 having an amino acid sequence 100% identical to the amino acid sequence of SEQ ID NO: 323. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 324 and comprising therein a complementarity determining region (CDR) L1 having 100% sequence identity to an amino acid sequence of S SEQ ID NO: 325, a CDR L2 having an amino acid sequence 100% identical to the amino acid sequence of STS, and a CDR L3 having an amino acid sequence 100% identical to the amino acid sequence of SEQ ID NO: 326. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 320 and comprising therein a complementarity determining region (CDR) H1 having an amino acid sequence 100% identical to the amino acid sequence of SEQ ID NO: 321, a CDR H2 having an amino acid sequence 100% identical to the amino acid sequence of SEQ ID NO: 322, and a CDR H3 having an amino acid sequence 100% identical to the amino acid sequence of SEQ ID NO: 323; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 324 and comprising therein a complementarity determining region (CDR) L1 having 100% sequence identity to an amino acid sequence of SEQ ID NO: 325, a CDR L2 having an amino acid sequence 100% identical to the amino acid sequence of STS, and a CDR L3 having an amino acid sequence 100% identical to the amino acid sequence of SEQ ID NO: 326.

In some embodiments, the antibody comprises a complementarity determining region (CDR) H1 having an amino acid sequence 100% identical to the amino acid sequence of SEQ ID NO: 328, a CDR H2 having an amino acid sequence 100% identical to the amino acid sequence of SEQ ID NO: 329, and a CDR H3 having an amino acid sequence 100% identical to the amino acid sequence of SEQ ID NO: 330. In some embodiments, the antibody comprises a complementarity determining region (CDR) L1 having 100% sequence identity to an amino acid sequence of SEQ ID NO: 332, a CDR L2 having an amino acid sequence 100% identical to the amino acid sequence DAS, and a CDR L3 having an amino acid sequence 100% identical to the amino acid sequence of SEQ ID NO: 333. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 327. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 331. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 327. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 331. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 327. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 331. In some embodiments, the antibody comprises a variable heavy chain (VH) region having an amino acid sequence with at least 95% (e.g., at least 95%, 96%, 97%, 98%, 99%, or more) identity to SEQ ID NO: 327. In some embodiments, the antibody comprises a variable light chain (VL) region having an amino acid sequence with at least 95% (e.g., at least 95%, 96%, 97%, 98%, 99%, or more) identity to SEQ ID NO: 331. In some embodiments, the antibody comprises a variable heavy chain (VH) region having an amino acid sequence 100% identical with the amino acid sequence of SEQ ID NO: 327. In some embodiments, the antibody comprises a variable light chain (VL) region having an amino acid sequence 100% identical with the amino acid sequence of SEQ ID NO: 331. In some embodiments, the antibody comprises: (a) a complementarity determining region (CDR) H1 having an amino acid sequence 100% identical to the amino acid sequence of SEQ ID NO: 328, a CDR H2 having an amino acid sequence 100% identical to the amino acid sequence of SEQ ID NO: 329, and a CDR H3 having an amino acid sequence 100% identical to the amino acid sequence of SEQ ID NO: 330; and (b) a complementarity determining region (CDR) L1 having 100% sequence identity to an amino acid sequence of SEQ ID NO: 332, a CDR L2 having an amino acid sequence 100% identical to the amino acid sequence DAS, and a CDR L3 having an amino acid sequence 100% identical to the amino acid sequence of SEQ ID NO: 333. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 327 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 331. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 327 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 331. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 327 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 331. In some embodiments, the antibody comprises a variable heavy chain (VH) region having an amino acid sequence with at least 95% (e.g., at least 95%, 96%, 97%, 98%, 99%, or more) identity to SEQ ID NO: 327 and a variable light chain (VL) region having an amino acid sequence with at least 95% (e.g., at least 95%, 96%, 97%, 98%, 99%, or more) identity to SEQ ID NO: 331. In some embodiments, the antibody comprises a variable heavy chain (VH) region having an amino acid sequence 100% identical with the amino acid sequence of SEQ ID NO: 327 and a variable light chain (VL) region having an amino acid sequence 100% identical with the amino acid sequence of SEQ ID NO: 331. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 327 and comprising therein a complementarity determining region (CDR) H1 having an amino acid sequence 100% identical to the amino acid sequence of SEQ ID NO: 328, a CDR H2 having an amino acid sequence 100% identical to the amino acid sequence of SEQ ID NO: 329, and a CDR H3 having an amino acid sequence 100% identical to the amino acid sequence of SEQ ID NO: 330. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 331 and comprising therein a complementarity determining region (CDR) L1 having 100% sequence identity to an amino acid sequence of SEQ ID NO: 332, a CDR L2 having an amino acid sequence 100% identical to the amino acid sequence DAS, and a CDR L3 having an amino acid sequence 100% identical to the amino acid sequence of SEQ ID NO: 333. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 327 and comprising therein a complementarity determining region (CDR) H1 having an amino acid sequence 100% identical to the amino acid sequence of SEQ ID NO: 328, a CDR H2 having an amino acid sequence 100% identical to the amino acid sequence of SEQ ID NO: 329, and a CDR H3 having an amino acid sequence 100% identical to the amino acid sequence of SEQ ID NO: 330; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 331 and comprising therein a complementarity determining region (CDR) L1 having 100% sequence identity to an amino acid sequence of SEQ ID NO: 332, a CDR L2 having an amino acid sequence 100% identical to the amino acid sequence DAS, and a CDR L3 having an amino acid sequence 100% identical to the amino acid sequence of SEQ ID NO: 333.

In some embodiments, the antibody comprises a complementarity determining region (CDR) H1 having an amino acid sequence 100% identical to the amino acid sequence of SEQ ID NO: 335, a CDR H2 having an amino acid sequence 100% identical to the amino acid sequence of SEQ ID NO: 336, and a CDR H3 having an amino acid sequence 100% identical to the amino acid sequence of SEQ ID NO: 337. In some embodiments, the antibody comprises a complementarity determining region (CDR) L1 having 100% sequence identity to an amino acid sequence of SEQ ID NO: 339, a CDR L2 having an amino acid sequence 100% identical to the amino acid sequence of GAS, and a CDR L3 having an amino acid sequence 100% identical to the amino acid sequence of SEQ ID NO: 340. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 334. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 338. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 334. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 338. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 334. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 338. In some embodiments, the antibody comprises a variable heavy chain (VH) region having an amino acid sequence with at least 95% (e.g., at least 95%, 96%, 97%, 98%, 99%, or more) identity to SEQ ID NO: 334. In some embodiments, the antibody comprises a variable light chain (VL) region having an amino acid sequence with at least 95% (e.g., at least 95%, 96%, 97%, 98%, 99%, or more) identity to SEQ ID NO: 338. In some embodiments, the antibody comprises a variable heavy chain (VH) region having an amino acid sequence 100% identical with the amino acid sequence of SEQ ID NO: 334. In some embodiments, the antibody comprises a variable light chain (VL) region having an amino acid sequence 100% identical with the amino acid sequence of SEQ ID NO: 338. In some embodiments, the antibody comprises: (a) a complementarity determining region (CDR) H1 having an amino acid sequence 100% identical to the amino acid sequence of SEQ ID NO: 335, a CDR H2 having an amino acid sequence 100% identical to the amino acid sequence of SEQ ID NO: 336, and a CDR H3 having an amino acid sequence 100% identical to the amino acid sequence of SEQ ID NO: 337; and (b) a complementarity determining region (CDR) L1 having 100% sequence identity to an amino acid sequence of SEQ ID NO: 339, a CDR L2 having an amino acid sequence 100% identical to the amino acid sequence of GAS, and a CDR L3 having an amino acid sequence 100% identical to the amino acid sequence of SEQ ID NO: 340. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 334 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 338. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 334 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 338. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 334 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 338. In some embodiments, the antibody comprises a variable heavy chain (VH) region having an amino acid sequence with at least 95% (e.g., at least 95%, 96%, 97%, 98%, 99%, or more) identity to SEQ ID NO: 334 and a variable light chain (VL) region having an amino acid sequence with at least 95% (e.g., at least 95%, 96%, 97%, 98%, 99%, or more) identity to SEQ ID NO: 338. In some embodiments, the antibody comprises a variable heavy chain (VH) region having an amino acid sequence 100% identical with the amino acid sequence of SEQ ID NO: 334 and a variable light chain (VL) region having an amino acid sequence 100% identical with the amino acid sequence of SEQ ID NO: 338. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 334 and comprising therein a complementarity determining region (CDR) H1 having an amino acid sequence 100% identical to the amino acid sequence of SEQ ID NO: 335, a CDR H2 having an amino acid sequence 100% identical to the amino acid sequence of SEQ ID NO: 336, and a CDR H3 having an amino acid sequence 100% identical to the amino acid sequence of SEQ ID NO: 337. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 338 and comprising therein a complementarity determining region (CDR) L1 having 100% sequence identity to an amino acid sequence of SEQ ID NO: 339, a CDR L2 having an amino acid sequence 100% identical to the amino acid sequence of GAS, and a CDR L3 having an amino acid sequence 100% identical to the amino acid sequence of SEQ ID NO: 340. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 334 and comprising therein a complementarity determining region (CDR) H1 having an amino acid sequence 100% identical to the amino acid sequence of SEQ ID NO: 335, a CDR H2 having an amino acid sequence 100% identical to the amino acid sequence of SEQ ID NO: 336, and a CDR H3 having an amino acid sequence 100% identical to the amino acid sequence of SEQ ID NO: 337; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 338 and comprising therein a complementarity determining region (CDR) L1 having 100% sequence identity to an amino acid sequence of SEQ ID NO: 339, a CDR L2 having an amino acid sequence 100% identical to the amino acid sequence of GAS, and a CDR L3 having an amino acid sequence 100% identical to the amino acid sequence of SEQ ID NO: 340.

In some embodiments, the antibody comprises a complementarity determining region (CDR) H1 having an amino acid sequence 100% identical to the amino acid sequence of SEQ ID NO: 342, a CDR H2 having an amino acid sequence 100% identical to the amino acid sequence of SEQ ID NO: 343, and a CDR H3 having an amino acid sequence 100% identical to the amino acid sequence of SEQ ID NO: 344. In some embodiments, the antibody comprises a complementarity determining region (CDR) L1 having 100% sequence identity to an amino acid sequence of SEQ ID NO: 346, a CDR L2 having an amino acid sequence 100% identical to the amino acid sequence of DAS, and a CDR L3 having an amino acid sequence 100% identical to the amino acid sequence of SEQ ID NO: 347. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 341. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 345. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 341. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 345. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 341. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 345. In some embodiments, the antibody comprises a variable heavy chain (VH) region having an amino acid sequence with at least 95% (e.g., at least 95%, 96%, 97%, 98%, 99%, or more) identity to SEQ ID NO: 341. In some embodiments, the antibody comprises a variable light chain (VL) region having an amino acid sequence with at least 95% (e.g., at least 95%, 96%, 97%, 98%, 99%, or more) identity to SEQ ID NO: 345. In some embodiments, the antibody comprises a variable heavy chain (VH) region having an amino acid sequence 100% identical with the amino acid sequence of SEQ ID NO: 341. In some embodiments, the antibody comprises a variable light chain (VL) region having an amino acid sequence 100% identical with the amino acid sequence of SEQ ID NO: 345. In some embodiments, the antibody comprises: (a) a complementarity determining region (CDR) H1 having an amino acid sequence 100% identical to the amino acid sequence of SEQ ID NO: 342, a CDR H2 having an amino acid sequence 100% identical to the amino acid sequence of SEQ ID NO: 343, and a CDR H3 having an amino acid sequence 100% identical to the amino acid sequence of SEQ ID NO: 344; and (b) a complementarity determining region (CDR) L1 having 100% sequence identity to an amino acid sequence of SEQ ID NO: 346, a CDR L2 having an amino acid sequence 100% identical to the amino acid sequence of DAS, and a CDR L3 having an amino acid sequence 100% identical to the amino acid sequence of SEQ ID NO: 347. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 341 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 345. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 341 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 345. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 341 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 345. In some embodiments, the antibody comprises a variable heavy chain (VH) region having an amino acid sequence with at least 95% (e.g., at least 95%, 96%, 97%, 98%, 99%, or more) identity to SEQ ID NO: 341 and a variable light chain (VL) region having an amino acid sequence with at least 95% (e.g., at least 95%, 96%, 97%, 98%, 99%, or more) identity to SEQ ID NO: 345. In some embodiments, the antibody comprises a variable heavy chain (VH) region having an amino acid sequence 100% identical with the amino acid sequence of SEQ ID NO: 341 and a variable light chain (VL) region having an amino acid sequence 100% identical with the amino acid sequence of SEQ ID NO: 345. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 341 and comprising therein a complementarity determining region (CDR) H1 having an amino acid sequence 100% identical to the amino acid sequence of SEQ ID NO: 342, a CDR H2 having an amino acid sequence 100% identical to the amino acid sequence of SEQ ID NO: 343, and a CDR H3 having an amino acid sequence 100% identical to the amino acid sequence of SEQ ID NO: 344. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 345 and comprising therein a complementarity determining region (CDR) L1 having 100% sequence identity to an amino acid sequence of SEQ ID NO: 346, a CDR L2 having an amino acid sequence 100% identical to the amino acid sequence of DAS, and a CDR L3 having an amino acid sequence 100% identical to the amino acid sequence of SEQ ID NO: 347. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 341 and comprising therein a complementarity determining region (CDR) H1 having an amino acid sequence 100% identical to the amino acid sequence of SEQ ID NO: 342, a CDR H2 having an amino acid sequence 100% identical to the amino acid sequence of SEQ ID NO: 343, and a CDR H3 having an amino acid sequence 100% identical to the amino acid sequence of SEQ ID NO: 344; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 345 and comprising therein a complementarity determining region (CDR) L1 having 100% sequence identity to an amino acid sequence of SEQ ID NO: 346, a CDR L2 having an amino acid sequence 100% identical to the amino acid sequence of DAS, and a CDR L3 having an amino acid sequence 100% identical to the amino acid sequence of SEQ ID NO: 347.

In some embodiments, the antibody comprises a complementarity determining region (CDR) H1 having an amino acid sequence 100% identical to the amino acid sequence of SEQ ID NO: 349, a CDR H2 having an amino acid sequence 100% identical to the amino acid sequence of SEQ ID NO: 350, and a CDR H3 having an amino acid sequence 100% identical to the amino acid sequence of SEQ ID NO: 351. In some embodiments, the antibody comprises a complementarity determining region (CDR) L1 having 100% sequence identity to an amino acid sequence of SEQ ID NO: 353, a CDR L2 having an amino acid sequence 100% identical to the amino acid sequence of DTS, and a CDR L3 having an amino acid sequence 100% identical to the amino acid sequence of SEQ ID NO: 354. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 348. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 352. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 348. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 352. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 348. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 352. In some embodiments, the antibody comprises a variable heavy chain (VH) region having an amino acid sequence with at least 95% (e.g., at least 95%, 96%, 97%, 98%, 99%, or more) identity to SEQ ID NO: 348. In some embodiments, the antibody comprises a variable light chain (VL) region having an amino acid sequence with at least 95% (e.g., at least 95%, 96%, 97%, 98%, 99%, or more) identity to SEQ ID NO: 352. In some embodiments, the antibody comprises a variable heavy chain (VH) region having an amino acid sequence 100% identical with the amino acid sequence of SEQ ID NO: 348. In some embodiments, the antibody comprises a variable light chain (VL) region having an amino acid sequence 100% identical with the amino acid sequence of SEQ ID NO: 352. In some embodiments, the antibody comprises: (a) a complementarity determining region (CDR) H1 having an amino acid sequence 100% identical to the amino acid sequence of SEQ ID NO: 349, a CDR H2 having an amino acid sequence 100% identical to the amino acid sequence of SEQ ID NO: 350, and a CDR H3 having an amino acid sequence 100% identical to the amino acid sequence of SEQ ID NO: 351; and (b) a complementarity determining region (CDR) L1 having 100% sequence identity to an amino acid sequence of SEQ ID NO: 353, a CDR L2 having an amino acid sequence 100% identical to the amino acid sequence of DTS, and a CDR L3 having an amino acid sequence 100% identical to the amino acid sequence of SEQ ID NO: 354. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 348 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 352. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 348 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 352. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 348 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 352. In some embodiments, the antibody comprises a variable heavy chain (VH) region having an amino acid sequence with at least 95% (e.g., at least 95%, 96%, 97%, 98%, 99%, or more) identity to SEQ ID NO: 348 and a variable light chain (VL) region having an amino acid sequence with at least 95% (e.g., at least 95%, 96%, 97%, 98%, 99%, or more) identity to SEQ ID NO: 352. In some embodiments, the antibody comprises a variable heavy chain (VH) region having an amino acid sequence 100% identical with the amino acid sequence of SEQ ID NO: 348 and a variable light chain (VL) region having an amino acid sequence 100% identical with the amino acid sequence of SEQ ID NO: 352. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 348 and comprising therein a complementarity determining region (CDR) H1 having an amino acid sequence 100% identical to the amino acid sequence of SEQ ID NO: 349, a CDR H2 having an amino acid sequence 100% identical to the amino acid sequence of SEQ ID NO: 350, and a CDR H3 having an amino acid sequence 100% identical to the amino acid sequence of SEQ ID NO: 351. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 352 and comprising therein a complementarity determining region (CDR) L1 having 100% sequence identity to an amino acid sequence of SEQ ID NO: 353, a CDR L2 having an amino acid sequence 100% identical to the amino acid sequence of DTS, and a CDR L3 having an amino acid sequence 100% identical to the amino acid sequence of SEQ ID NO: 354. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 348 and comprising therein a complementarity determining region (CDR) H1 having an amino acid sequence 100% identical to the amino acid sequence of SEQ ID NO: 349, a CDR H2 having an amino acid sequence 100% identical to the amino acid sequence of SEQ ID NO: 350, and a CDR H3 having an amino acid sequence 100% identical to the amino acid sequence of SEQ ID NO: 351; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 352 and comprising therein a complementarity determining region (CDR) L1 having 100% sequence identity to an amino acid sequence of SEQ ID NO: 353, a CDR L2 having an amino acid sequence 100% identical to the amino acid sequence of DTS, and a CDR L3 having an amino acid sequence 100% identical to the amino acid sequence of SEQ ID NO: 354.

In some embodiments, the antibody comprises a complementarity determining region (CDR) H1 having an amino acid sequence 100% identical to the amino acid sequence of SEQ ID NO: 356, a CDR H2 having an amino acid sequence 100% identical to the amino acid sequence of SEQ ID NO: 357, and a CDR H3 having an amino acid sequence 100% identical to the amino acid sequence of SEQ ID NO: 358. In some embodiments, the antibody comprises a complementarity determining region (CDR) L1 having 100% sequence identity to an amino acid sequence of SEQ ID NO: 360, a CDR L2 having an amino acid sequence 100% identical to the amino acid sequence of AAS, and a CDR L3 having an amino acid sequence 100% identical to the amino acid sequence of SEQ ID NO: 361. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 355. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 359. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 355. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 359. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 355. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 359. In some embodiments, the antibody comprises a variable heavy chain (VH) region having an amino acid sequence with at least 95% (e.g., at least 95%, 96%, 97%, 98%, 99%, or more) identity to SEQ ID NO: 355. In some embodiments, the antibody comprises a variable light chain (VL) region having an amino acid sequence with at least 95% (e.g., at least 95%, 96%, 97%, 98%, 99%, or more) identity to SEQ ID NO: 359. In some embodiments, the antibody comprises a variable heavy chain (VH) region having an amino acid sequence 100% identical with the amino acid sequence of SEQ ID NO: 355. In some embodiments, the antibody comprises a variable light chain (VL) region having an amino acid sequence 100% identical with the amino acid sequence of SEQ ID NO: 359. In some embodiments, the antibody comprises: (a) a complementarity determining region (CDR) H1 having an amino acid sequence 100% identical to the amino acid sequence of SEQ ID NO: 356, a CDR H2 having an amino acid sequence 100% identical to the amino acid sequence of SEQ ID NO: 357, and a CDR H3 having an amino acid sequence 100% identical to the amino acid sequence of SEQ ID NO: 358; and (b) a complementarity determining region (CDR) L1 having 100% sequence identity to an amino acid sequence of SEQ ID NO: 360, a CDR L2 having an amino acid sequence 100% identical to the amino acid sequence of AAS, and a CDR L3 having an amino acid sequence 100% identical to the amino acid sequence of SEQ ID NO: 361. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 355 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 359. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 355 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 359. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 355 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 359. In some embodiments, the antibody comprises a variable heavy chain (VH) region having an amino acid sequence with at least 95% (e.g., at least 95%, 96%, 97%, 98%, 99%, or more) identity to SEQ ID NO: 355 and a variable light chain (VL) region having an amino acid sequence with at least 95% (e.g., at least 95%, 96%, 97%, 98%, 99%, or more) identity to SEQ ID NO: 359. In some embodiments, the antibody comprises a variable heavy chain (VH) region having an amino acid sequence 100% identical with the amino acid sequence of SEQ ID NO: 355 and a variable light chain (VL) region having an amino acid sequence 100% identical with the amino acid sequence of SEQ ID NO: 359. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 355 and comprising therein a complementarity determining region (CDR) H1 having an amino acid sequence 100% identical to the amino acid sequence of SEQ ID NO: 356, a CDR H2 having an amino acid sequence 100% identical to the amino acid sequence of SEQ ID NO: 357, and a CDR H3 having an amino acid sequence 100% identical to the amino acid sequence of SEQ ID NO: 358. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 359 and comprising therein a complementarity determining region (CDR) L1 having 100% sequence identity to an amino acid sequence of SEQ ID NO: 360, a CDR L2 having an amino acid sequence 100% identical to the amino acid sequence of AAS, and a CDR L3 having an amino acid sequence 100% identical to the amino acid sequence of SEQ ID NO: 361. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 355 and comprising therein a complementarity determining region (CDR) H1 having an amino acid sequence 100% identical to the amino acid sequence of SEQ ID NO: 356, a CDR H2 having an amino acid sequence 100% identical to the amino acid sequence of SEQ ID NO: 357, and a CDR H3 having an amino acid sequence 100% identical to the amino acid sequence of SEQ ID NO: 358; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 359 and comprising therein a complementarity determining region (CDR) L1 having 100% sequence identity to an amino acid sequence of SEQ ID NO: 360, a CDR L2 having an amino acid sequence 100% identical to the amino acid sequence of AAS, and a CDR L3 having an amino acid sequence 100% identical to the amino acid sequence of SEQ ID NO: 361.

In some embodiments, the antibody comprises a complementarity determining region (CDR) H1 having an amino acid sequence 100% identical to the amino acid sequence of SEQ ID NO: 363, a CDR H2 having an amino acid sequence 100% identical to the amino acid sequence of SEQ ID NO: 364, and a CDR H3 having an amino acid sequence 100% identical to the amino acid sequence of SEQ ID NO: 365. In some embodiments, the antibody comprises a complementarity determining region (CDR) L1 having 100% sequence identity to an amino acid sequence of SEQ ID NO: 367, a CDR L2 having an amino acid sequence 100% identical to the amino acid sequence of AAS, and a CDR L3 having an amino acid sequence 100% identical to the amino acid sequence of SEQ ID NO: 368. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 362. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 366. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 362. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 366. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 362. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 366. In some embodiments, the antibody comprises a variable heavy chain (VH) region having an amino acid sequence with at least 95% (e.g., at least 95%, 96%, 97%, 98%, 99%, or more) identity to SEQ ID NO: 362. In some embodiments, the antibody comprises a variable light chain (VL) region having an amino acid sequence with at least 95% (e.g., at least 95%, 96%, 97%, 98%, 99%, or more) identity to SEQ ID NO: 366. In some embodiments, the antibody comprises a variable heavy chain (VH) region having an amino acid sequence 100% identical with the amino acid sequence of SEQ ID NO: 362. In some embodiments, the antibody comprises a variable light chain (VL) region having an amino acid sequence 100% identical with the amino acid sequence of SEQ ID NO: 366. In some embodiments, the antibody comprises: (a) a complementarity determining region (CDR) H1 having an amino acid sequence 100% identical to the amino acid sequence of SEQ ID NO: 363, a CDR H2 having an amino acid sequence 100% identical to the amino acid sequence of SEQ ID NO: 364, and a CDR H3 having an amino acid sequence 100% identical to the amino acid sequence of SEQ ID NO: 365; and (b) a complementarity determining region (CDR) L1 having 100% sequence identity to an amino acid sequence of SEQ ID NO: 367, a CDR L2 having an amino acid sequence 100% identical to the amino acid sequence of AAS, and a CDR L3 having an amino acid sequence 100% identical to the amino acid sequence of SEQ ID NO: 368. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 362 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 366. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 362 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 366. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 362 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 366. In some embodiments, the antibody comprises a variable heavy chain (VH) region having an amino acid sequence with at least 95% (e.g., at least 95%, 96%, 97%, 98%, 99%, or more) identity to SEQ ID NO: 362 and a variable light chain (VL) region having an amino acid sequence with at least 95% (e.g., at least 95%, 96%, 97%, 98%, 99%, or more) identity to SEQ ID NO: 366. In some embodiments, the antibody comprises a variable heavy chain (VH) region having an amino acid sequence 100% identical with the amino acid sequence of SEQ ID NO: 362 and a variable light chain (VL) region having an amino acid sequence 100% identical with the amino acid sequence of SEQ ID NO: 366. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 362 and comprising therein a complementarity determining region (CDR) H1 having an amino acid sequence 100% identical to the amino acid sequence of SEQ ID NO: 363, a CDR H2 having an amino acid sequence 100% identical to the amino acid sequence of SEQ ID NO: 364, and a CDR H3 having an amino acid sequence 100% identical to the amino acid sequence of SEQ ID NO: 365. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 366 and comprising therein a complementarity determining region (CDR) L1 having 100% sequence identity to an amino acid sequence of SEQ ID NO: 367, a CDR L2 having an amino acid sequence 100% identical to the amino acid sequence of AAS, and a CDR L3 having an amino acid sequence 100% identical to the amino acid sequence of SEQ ID NO: 368. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 362 and comprising therein a complementarity determining region (CDR) H1 having an amino acid sequence 100% identical to the amino acid sequence of SEQ ID NO: 363, a CDR H2 having an amino acid sequence 100% identical to the amino acid sequence of SEQ ID NO: 364, and a CDR H3 having an amino acid sequence 100% identical to the amino acid sequence of SEQ ID NO: 365; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 366 and comprising therein a complementarity determining region (CDR) L1 having 100% sequence identity to an amino acid sequence of SEQ ID NO: 367, a CDR L2 having an amino acid sequence 100% identical to the amino acid sequence of AAS, and a CDR L3 having an amino acid sequence 100% identical to the amino acid sequence of SEQ ID NO: 368.

In some embodiments, the antibody comprises a complementarity determining region (CDR) H1 having an amino acid sequence 100% identical to the amino acid sequence of SEQ ID NO: 370, a CDR H2 having an amino acid sequence 100% identical to the amino acid sequence of SEQ ID NO: 371, and a CDR H3 having an amino acid sequence 100% identical to the amino acid sequence of SEQ ID NO: 372. In some embodiments, the antibody comprises a complementarity determining region (CDR) L1 having 100% sequence identity to an amino acid sequence of SEQ ID NO: 374, a CDR L2 having an amino acid sequence 100% identical to the amino acid sequence of GTS, and a CDR L3 having an amino acid sequence 100% identical to the amino acid sequence of SEQ ID NO: 375. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 369. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 373. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 369. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 373. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 369. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 373. In some embodiments, the antibody comprises a variable heavy chain (VH) region having an amino acid sequence with at least 95% (e.g., at least 95%, 96%, 97%, 98%, 99%, or more) identity to SEQ ID NO: 369. In some embodiments, the antibody comprises a variable light chain (VL) region having an amino acid sequence with at least 95% (e.g., at least 95%, 96%, 97%, 98%, 99%, or more) identity to SEQ ID NO: 373. In some embodiments, the antibody comprises a variable heavy chain (VH) region having an amino acid sequence 100% identical with the amino acid sequence of SEQ ID NO: 369. In some embodiments, the antibody comprises a variable light chain (VL) region having an amino acid sequence 100% identical with the amino acid sequence of SEQ ID NO: 373. In some embodiments, the antibody comprises: (a) a complementarity determining region (CDR) H1 having an amino acid sequence 100% identical to the amino acid sequence of SEQ ID NO: 370, a CDR H2 having an amino acid sequence 100% identical to the amino acid sequence of SEQ ID NO: 371, and a CDR H3 having an amino acid sequence 100% identical to the amino acid sequence of SEQ ID NO: 372; and (b) a complementarity determining region (CDR) L1 having 100% sequence identity to an amino acid sequence of SEQ ID NO: 374, a CDR L2 having an amino acid sequence 100% identical to the amino acid sequence of GTS, and a CDR L3 having an amino acid sequence 100% identical to the amino acid sequence of SEQ ID NO: 375. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 369 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 373. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 369 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 373. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 369 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 373. In some embodiments, the antibody comprises a variable heavy chain (VH) region having an amino acid sequence with at least 95% (e.g., at least 95%, 96%, 97%, 98%, 99%, or more) identity to SEQ ID NO: 369 and a variable light chain (VL) region having an amino acid sequence with at least 95% (e.g., at least 95%, 96%, 97%, 98%, 99%, or more) identity to SEQ ID NO: 373. In some embodiments, the antibody comprises a variable heavy chain (VH) region having an amino acid sequence 100% identical with the amino acid sequence of SEQ ID NO: 369 and a variable light chain (VL) region having an amino acid sequence 100% identical with the amino acid sequence of SEQ ID NO: 373. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 369 and comprising therein a complementarity determining region (CDR) H1 having an amino acid sequence 100% identical to the amino acid sequence of SEQ ID NO: 370, a CDR H2 having an amino acid sequence 100% identical to the amino acid sequence of SEQ ID NO: 371, and a CDR H3 having an amino acid sequence 100% identical to the amino acid sequence of SEQ ID NO: 372. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 373 and comprising therein a complementarity determining region (CDR) L1 having 100% sequence identity to an amino acid sequence of SEQ ID NO: 374, a CDR L2 having an amino acid sequence 100% identical to the amino acid sequence of GTS, and a CDR L3 having an amino acid sequence 100% identical to the amino acid sequence of SEQ ID NO: 375. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 369 and comprising therein a complementarity determining region (CDR) H1 having an amino acid sequence 100% identical to the amino acid sequence of SEQ ID NO: 370, a CDR H2 having an amino acid sequence 100% identical to the amino acid sequence of SEQ ID NO: 371, and a CDR H3 having an amino acid sequence 100% identical to the amino acid sequence of SEQ ID NO: 372; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 373 and comprising therein a complementarity determining region (CDR) L1 having 100% sequence identity to an amino acid sequence of SEQ ID NO: 374, a CDR L2 having an amino acid sequence 100% identical to the amino acid sequence of GTS, and a CDR L3 having an amino acid sequence 100% identical to the amino acid sequence of SEQ ID NO: 375.

In some embodiments, the antibody comprises a complementarity determining region (CDR) H1 having an amino acid sequence 100% identical to the amino acid sequence of SEQ ID NO: 377, a CDR H2 having an amino acid sequence 100% identical to the amino acid sequence of SEQ ID NO: 378, and a CDR H3 having an amino acid sequence 100% identical to the amino acid sequence of SEQ ID NO: 379. In some embodiments, the antibody comprises a complementarity determining region (CDR) L1 having 100% sequence identity to an amino acid sequence of SEQ ID NO: 381, a CDR L2 having an amino acid sequence 100% identical to the amino acid sequence of ETS, and a CDR L3 having an amino acid sequence 100% identical to the amino acid sequence of SEQ ID NO: 382. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 376. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 380. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 376. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 380. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 376. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 380. In some embodiments, the antibody comprises a variable heavy chain (VH) region having an amino acid sequence with at least 95% (e.g., at least 95%, 96%, 97%, 98%, 99%, or more) identity to SEQ ID NO: 376. In some embodiments, the antibody comprises a variable light chain (VL) region having an amino acid sequence with at least 95% (e.g., at least 95%, 96%, 97%, 98%, 99%, or more) identity to SEQ ID NO: 380. In some embodiments, the antibody comprises a variable heavy chain (VH) region having an amino acid sequence 100% identical with the amino acid sequence of SEQ ID NO: 376. In some embodiments, the antibody comprises a variable light chain (VL) region having an amino acid sequence 100% identical with the amino acid sequence of SEQ ID NO: 380. In some embodiments, the antibody comprises: (a) a complementarity determining region (CDR) H1 having an amino acid sequence 100% identical to the amino acid sequence of SEQ ID NO: 377, a CDR H2 having an amino acid sequence 100% identical to the amino acid sequence of SEQ ID NO: 378, and a CDR H3 having an amino acid sequence 100% identical to the amino acid sequence of SEQ ID NO: 379; and (b) a complementarity determining region (CDR) L1 having 100% sequence identity to an amino acid sequence of SEQ ID NO: 381, a CDR L2 having an amino acid sequence 100% identical to the amino acid sequence of ETS, and a CDR L3 having an amino acid sequence 100% identical to the amino acid sequence of SEQ ID NO: 382. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 376 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 380. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 376 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 380. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 376 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 380. In some embodiments, the antibody comprises a variable heavy chain (VH) region having an amino acid sequence with at least 95% (e.g., at least 95%, 96%, 97%, 98%, 99%, or more) identity to SEQ ID NO: 376 and a variable light chain (VL) region having an amino acid sequence with at least 95% (e.g., at least 95%, 96%, 97%, 98%, 99%, or more) identity to SEQ ID NO: 380. In some embodiments, the antibody comprises a variable heavy chain (VH) region having an amino acid sequence 100% identical with the amino acid sequence of SEQ ID NO: 376 and a variable light chain (VL) region having an amino acid sequence 100% identical with the amino acid sequence of SEQ ID NO: 380. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 376 and comprising therein a complementarity determining region (CDR) H1 having an amino acid sequence 100% identical to the amino acid sequence of SEQ ID NO: 377, a CDR H2 having an amino acid sequence 100% identical to the amino acid sequence of SEQ ID NO: 378, and a CDR H3 having an amino acid sequence 100% identical to the amino acid sequence of SEQ ID NO: 379. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 380 and comprising therein a complementarity determining region (CDR) L1 having 100% sequence identity to an amino acid sequence of SEQ ID NO: 381, a CDR L2 having an amino acid sequence 100% identical to the amino acid sequence of ETS, and a CDR L3 having an amino acid sequence 100% identical to the amino acid sequence of SEQ ID NO: 382. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 376 and comprising therein a complementarity determining region (CDR) H1 having an amino acid sequence 100% identical to the amino acid sequence of SEQ ID NO: 377, a CDR H2 having an amino acid sequence 100% identical to the amino acid sequence of SEQ ID NO: 378, and a CDR H3 having an amino acid sequence 100% identical to the amino acid sequence of SEQ ID NO: 379; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 380 and comprising therein a complementarity determining region (CDR) L1 having 100% sequence identity to an amino acid sequence of SEQ ID NO: 381, a CDR L2 having an amino acid sequence 100% identical to the amino acid sequence of ETS, and a CDR L3 having an amino acid sequence 100% identical to the amino acid sequence of SEQ ID NO: 382.

In some embodiments, the antibody comprises a complementarity determining region (CDR) H1 having an amino acid sequence 100% identical to the amino acid sequence of SEQ ID NO: 384, a CDR H2 having an amino acid sequence 100% identical to the amino acid sequence of SEQ ID NO: 385, and a CDR H3 having an amino acid sequence 100% identical to the amino acid sequence of SEQ ID NO: 386. In some embodiments, the antibody comprises a complementarity determining region (CDR) L1 having 100% sequence identity to an amino acid sequence of SEQ ID NO: 388, a CDR L2 having an amino acid sequence 100% identical to the amino acid sequence of GAS, and a CDR L3 having an amino acid sequence 100% identical to the amino acid sequence of SEQ ID NO: 389. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 383. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 387. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 383. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 387. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 383. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 387. In some embodiments, the antibody comprises a variable heavy chain (VH) region having an amino acid sequence with at least 95% (e.g., at least 95%, 96%, 97%, 98%, 99%, or more) identity to SEQ ID NO: 383. In some embodiments, the antibody comprises a variable light chain (VL) region having an amino acid sequence with at least 95% (e.g., at least 95%, 96%, 97%, 98%, 99%, or more) identity to SEQ ID NO: 387. In some embodiments, the antibody comprises a variable heavy chain (VH) region having an amino acid sequence 100% identical with the amino acid sequence of SEQ ID NO: 383. In some embodiments, the antibody comprises a variable light chain (VL) region having an amino acid sequence 100% identical with the amino acid sequence of SEQ ID NO: 387. In some embodiments, the antibody comprises: (a) a complementarity determining region (CDR) H1 having an amino acid sequence 100% identical to the amino acid sequence of SEQ ID NO: 384, a CDR H2 having an amino acid sequence 100% identical to the amino acid sequence of SEQ ID NO: 385, and a CDR H3 having an amino acid sequence 100% identical to the amino acid sequence of SEQ ID NO: 386; and (b) a complementarity determining region (CDR) L1 having 100% sequence identity to an amino acid sequence of SEQ ID NO: 388, a CDR L2 having an amino acid sequence 100% identical to the amino acid sequence of GAS, and a CDR L3 having an amino acid sequence 100% identical to the amino acid sequence of SEQ ID NO: 389. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 383 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 387. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 383 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 387. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 383 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 387. In some embodiments, the antibody comprises a variable heavy chain (VH) region having an amino acid sequence with at least 95% (e.g., at least 95%, 96%, 97%, 98%, 99%, or more) identity to SEQ ID NO: 383 and a variable light chain (VL) region having an amino acid sequence with at least 95% (e.g., at least 95%, 96%, 97%, 98%, 99%, or more) identity to SEQ ID NO: 387. In some embodiments, the antibody comprises a variable heavy chain (VH) region having an amino acid sequence 100% identical with the amino acid sequence of SEQ ID NO: 383 and a variable light chain (VL) region having an amino acid sequence 100% identical with the amino acid sequence of SEQ ID NO: 387. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 383 and comprising therein a complementarity determining region (CDR) H1 having an amino acid sequence 100% identical to the amino acid sequence of SEQ ID NO: 384, a CDR H2 having an amino acid sequence 100% identical to the amino acid sequence of SEQ ID NO: 385, and a CDR H3 having an amino acid sequence 100% identical to the amino acid sequence of SEQ ID NO: 386. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 387 and comprising therein a complementarity determining region (CDR) L1 having 100% sequence identity to an amino acid sequence of SEQ ID NO: 388, a CDR L2 having an amino acid sequence 100% identical to the amino acid sequence of GAS, and a CDR L3 having an amino acid sequence 100% identical to the amino acid sequence of SEQ ID NO: 389. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 383 and comprising therein a complementarity determining region (CDR) H1 having an amino acid sequence 100% identical to the amino acid sequence of SEQ ID NO: 384, a CDR H2 having an amino acid sequence 100% identical to the amino acid sequence of SEQ ID NO: 385, and a CDR H3 having an amino acid sequence 100% identical to the amino acid sequence of SEQ ID NO: 386; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 387 and comprising therein a complementarity determining region (CDR) L1 having 100% sequence identity to an amino acid sequence of SEQ ID NO: 388, a CDR L2 having an amino acid sequence 100% identical to the amino acid sequence of GAS, and a CDR L3 having an amino acid sequence 100% identical to the amino acid sequence of SEQ ID NO: 389.

In some embodiments, the antibody comprises a complementarity determining region (CDR) H1 having an amino acid sequence 100% identical to the amino acid sequence of SEQ ID NO: 391, a CDR H2 having an amino acid sequence 100% identical to the amino acid sequence of SEQ ID NO: 392, and a CDR H3 having an amino acid sequence 100% identical to the amino acid sequence of SEQ ID NO: 393. In some embodiments, the antibody comprises a complementarity determining region (CDR) L1 having 100% sequence identity to an amino acid sequence of SEQ ID NO: 395, a CDR L2 having an amino acid sequence 100% identical to the amino acid sequence of DAS, and a CDR L3 having an amino acid sequence 100% identical to the amino acid sequence of SEQ ID NO: 396. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 390. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 394. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 390. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 394. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 390. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 394. In some embodiments, the antibody comprises a variable heavy chain (VH) region having an amino acid sequence with at least 95% (e.g., at least 95%, 96%, 97%, 98%, 99%, or more) identity to SEQ ID NO: 390. In some embodiments, the antibody comprises a variable light chain (VL) region having an amino acid sequence with at least 95% (e.g., at least 95%, 96%, 97%, 98%, 99%, or more) identity to SEQ ID NO: 394. In some embodiments, the antibody comprises a variable heavy chain (VH) region having an amino acid sequence 100% identical with the amino acid sequence of SEQ ID NO: 390. In some embodiments, the antibody comprises a variable light chain (VL) region having an amino acid sequence 100% identical with the amino acid sequence of SEQ ID NO: 394. In some embodiments, the antibody comprises: (a) a complementarity determining region (CDR) H1 having an amino acid sequence 100% identical to the amino acid sequence of SEQ ID NO: 391, a CDR H2 having an amino acid sequence 100% identical to the amino acid sequence of SEQ ID NO: 392, and a CDR H3 having an amino acid sequence 100% identical to the amino acid sequence of SEQ ID NO: 393; and (b) a complementarity determining region (CDR) L1 having 100% sequence identity to an amino acid sequence of SEQ ID NO: 395, a CDR L2 having an amino acid sequence 100% identical to the amino acid sequence of DAS, and a CDR L3 having an amino acid sequence 100% identical to the amino acid sequence of SEQ ID NO: 396. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 390 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 394. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 390 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 394. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 390 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 394. In some embodiments, the antibody comprises a variable heavy chain (VH) region having an amino acid sequence with at least 95% (e.g., at least 95%, 96%, 97%, 98%, 99%, or more) identity to SEQ ID NO: 390 and a variable light chain (VL) region having an amino acid sequence with at least 95% (e.g., at least 95%, 96%, 97%, 98%, 99%, or more) identity to SEQ ID NO: 394. In some embodiments, the antibody comprises a variable heavy chain (VH) region having an amino acid sequence 100% identical with the amino acid sequence of SEQ ID NO: 390 and a variable light chain (VL) region having an amino acid sequence 100% identical with the amino acid sequence of SEQ ID NO: 394. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 390 and comprising therein a complementarity determining region (CDR) H1 having an amino acid sequence 100% identical to the amino acid sequence of SEQ ID NO: 391, a CDR H2 having an amino acid sequence 100% identical to the amino acid sequence of SEQ ID NO: 392, and a CDR H3 having an amino acid sequence 100% identical to the amino acid sequence of SEQ ID NO: 393. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 394 and comprising therein a complementarity determining region (CDR) L1 having 100% sequence identity to an amino acid sequence of SEQ ID NO: 395, a CDR L2 having an amino acid sequence 100% identical to the amino acid sequence of DAS, and a CDR L3 having an amino acid sequence 100% identical to the amino acid sequence of SEQ ID NO: 396. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 390 and comprising therein a complementarity determining region (CDR) H1 having an amino acid sequence 100% identical to the amino acid sequence of SEQ ID NO: 391, a CDR H2 having an amino acid sequence 100% identical to the amino acid sequence of SEQ ID NO: 392, and a CDR H3 having an amino acid sequence 100% identical to the amino acid sequence of SEQ ID NO: 393; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 394 and comprising therein a complementarity determining region (CDR) L1 having 100% sequence identity to an amino acid sequence of SEQ ID NO: 395, a CDR L2 having an amino acid sequence 100% identical to the amino acid sequence of DAS, and a CDR L3 having an amino acid sequence 100% identical to the amino acid sequence of SEQ ID NO: 396. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 462. In some embodiments, the antibody comprises a second heavy chain region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 482. In some embodiments, the antibody comprises a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 472. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 462, a second heavy chain region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 482, and a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 472.

In some embodiments, the antibody comprises a complementarity determining region (CDR) H1 having an amino acid sequence 100% identical to the amino acid sequence of SEQ ID NO: 398, a CDR H2 having an amino acid sequence 100% identical to the amino acid sequence of SEQ ID NO: 399, and a CDR H3 having an amino acid sequence 100% identical to the amino acid sequence of SEQ ID NO: 400. In some embodiments, the antibody comprises a complementarity determining region (CDR) L1 having 100% sequence identity to an amino acid sequence of SEQ ID NO: 402, a CDR L2 having an amino acid sequence 100% identical to the amino acid sequence of GAS, and a CDR L3 having an amino acid sequence 100% identical to the amino acid sequence of SEQ ID NO: 403. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 397. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 401. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 397. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 401. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 397. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 401. In some embodiments, the antibody comprises a variable heavy chain (VH) region having an amino acid sequence with at least 95% (e.g., at least 95%, 96%, 97%, 98%, 99%, or more) identity to SEQ ID NO: 397. In some embodiments, the antibody comprises a variable light chain (VL) region having an amino acid sequence with at least 95% (e.g., at least 95%, 96%, 97%, 98%, 99%, or more) identity to SEQ ID NO: 401. In some embodiments, the antibody comprises a variable heavy chain (VH) region having an amino acid sequence 100% identical with the amino acid sequence of SEQ ID NO: 397. In some embodiments, the antibody comprises a variable light chain (VL) region having an amino acid sequence 100% identical with the amino acid sequence of SEQ ID NO: 401. In some embodiments, the antibody comprises: (a) a complementarity determining region (CDR) H1 having an amino acid sequence 100% identical to the amino acid sequence of SEQ ID NO: 398, a CDR H2 having an amino acid sequence 100% identical to the amino acid sequence of SEQ ID NO: 399, and a CDR H3 having an amino acid sequence 100% identical to the amino acid sequence of SEQ ID NO: 400; and (b) a complementarity determining region (CDR) L1 having 100% sequence identity to an amino acid sequence of SEQ ID NO: 402, a CDR L2 having an amino acid sequence 100% identical to the amino acid sequence of GAS, and a CDR L3 having an amino acid sequence 100% identical to the amino acid sequence of SEQ ID NO: 403. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 397 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 401. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 397 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 401. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 397 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 401. In some embodiments, the antibody comprises a variable heavy chain (VH) region having an amino acid sequence with at least 95% (e.g., at least 95%, 96%, 97%, 98%, 99%, or more) identity to SEQ ID NO: 397 and a variable light chain (VL) region having an amino acid sequence with at least 95% (e.g., at least 95%, 96%, 97%, 98%, 99%, or more) identity to SEQ ID NO: 401. In some embodiments, the antibody comprises a variable heavy chain (VH) region having an amino acid sequence 100% identical with the amino acid sequence of SEQ ID NO: 397 and a variable light chain (VL) region having an amino acid sequence 100% identical with the amino acid sequence of SEQ ID NO: 401. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 397 and comprising therein a complementarity determining region (CDR) H1 having an amino acid sequence 100% identical to the amino acid sequence of SEQ ID NO: 398, a CDR H2 having an amino acid sequence 100% identical to the amino acid sequence of SEQ ID NO: 399, and a CDR H3 having an amino acid sequence 100% identical to the amino acid sequence of SEQ ID NO: 400. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 401 and comprising therein a complementarity determining region (CDR) L1 having 100% sequence identity to an amino acid sequence of SEQ ID NO: 402, a CDR L2 having an amino acid sequence 100% identical to the amino acid sequence of GAS, and a CDR L3 having an amino acid sequence 100% identical to the amino acid sequence of SEQ ID NO: 403. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 397 and comprising therein a complementarity determining region (CDR) H1 having an amino acid sequence 100% identical to the amino acid sequence of SEQ ID NO: 398, a CDR H2 having an amino acid sequence 100% identical to the amino acid sequence of SEQ ID NO: 399, and a CDR H3 having an amino acid sequence 100% identical to the amino acid sequence of SEQ ID NO: 400; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 401 and comprising therein a complementarity determining region (CDR) L1 having 100% sequence identity to an amino acid sequence of SEQ ID NO: 402, a CDR L2 having an amino acid sequence 100% identical to the amino acid sequence of GAS, and a CDR L3 having an amino acid sequence 100% identical to the amino acid sequence of SEQ ID NO: 403.

In some embodiments, the antibody comprises a complementarity determining region (CDR) H1 having an amino acid sequence 100% identical to the amino acid sequence of SEQ ID NO: 405, a CDR H2 having an amino acid sequence 100% identical to the amino acid sequence of SEQ ID NO: 406, and a CDR H3 having an amino acid sequence 100% identical to the amino acid sequence of SEQ ID NO: 407. In some embodiments, the antibody comprises a complementarity determining region (CDR) L1 having 100% sequence identity to an amino acid sequence of SEQ ID NO: 409, a CDR L2 having an amino acid sequence 100% identical to the amino acid sequence of DAS, and a CDR L3 having an amino acid sequence 100% identical to the amino acid sequence of SEQ ID NO: 410. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 404. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 408. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 404. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 408. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 404. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 408. In some embodiments, the antibody comprises a variable heavy chain (VH) region having an amino acid sequence with at least 95% (e.g., at least 95%, 96%, 97%, 98%, 99%, or more) identity to SEQ ID NO: 404. In some embodiments, the antibody comprises a variable light chain (VL) region having an amino acid sequence with at least 95% (e.g., at least 95%, 96%, 97%, 98%, 99%, or more) identity to SEQ ID NO: 408. In some embodiments, the antibody comprises a variable heavy chain (VH) region having an amino acid sequence 100% identical with the amino acid sequence of SEQ ID NO: 404. In some embodiments, the antibody comprises a variable light chain (VL) region having an amino acid sequence 100% identical with the amino acid sequence of SEQ ID NO: 408. In some embodiments, the antibody comprises: (a) a complementarity determining region (CDR) H1 having an amino acid sequence 100% identical to the amino acid sequence of SEQ ID NO: 405, a CDR H2 having an amino acid sequence 100% identical to the amino acid sequence of SEQ ID NO: 406, and a CDR H3 having an amino acid sequence 100% identical to the amino acid sequence of SEQ ID NO: 407; and (b) a complementarity determining region (CDR) L1 having 100% sequence identity to an amino acid sequence of SEQ ID NO: 409, a CDR L2 having an amino acid sequence 100% identical to the amino acid sequence of DAS, and a CDR L3 having an amino acid sequence 100% identical to the amino acid sequence of SEQ ID NO: 410. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 404 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 408. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 404 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 408. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 404 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 408. In some embodiments, the antibody comprises a variable heavy chain (VH) region having an amino acid sequence with at least 95% (e.g., at least 95%, 96%, 97%, 98%, 99%, or more) identity to SEQ ID NO: 404 and a variable light chain (VL) region having an amino acid sequence with at least 95% (e.g., at least 95%, 96%, 97%, 98%, 99%, or more) identity to SEQ ID NO: 408. In some embodiments, the antibody comprises a variable heavy chain (VH) region having an amino acid sequence 100% identical with the amino acid sequence of SEQ ID NO: 404 and a variable light chain (VL) region having an amino acid sequence 100% identical with the amino acid sequence of SEQ ID NO: 408. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 404 and comprising therein a complementarity determining region (CDR) H1 having an amino acid sequence 100% identical to the amino acid sequence of SEQ ID NO: 405, a CDR H2 having an amino acid sequence 100% identical to the amino acid sequence of SEQ ID NO: 406, and a CDR H3 having an amino acid sequence 100% identical to the amino acid sequence of SEQ ID NO: 407. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 408 and comprising therein a complementarity determining region (CDR) L1 having 100% sequence identity to an amino acid sequence of SEQ ID NO: 409, a CDR L2 having an amino acid sequence 100% identical to the amino acid sequence of DAS, and a CDR L3 having an amino acid sequence 100% identical to the amino acid sequence of SEQ ID NO: 410. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 404 and comprising therein a complementarity determining region (CDR) H1 having an amino acid sequence 100% identical to the amino acid sequence of SEQ ID NO: 405, a CDR H2 having an amino acid sequence 100% identical to the amino acid sequence of SEQ ID NO: 406, and a CDR H3 having an amino acid sequence 100% identical to the amino acid sequence of SEQ ID NO: 407; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 408 and comprising therein a complementarity determining region (CDR) L1 having 100% sequence identity to an amino acid sequence of SEQ ID NO: 409, a CDR L2 having an amino acid sequence 100% identical to the amino acid sequence of DAS, and a CDR L3 having an amino acid sequence 100% identical to the amino acid sequence of SEQ ID NO: 410.

In some embodiments, the antibody comprises a complementarity determining region (CDR) H1 having an amino acid sequence 100% identical to the amino acid sequence of SEQ ID NO: 412, a CDR H2 having an amino acid sequence 100% identical to the amino acid sequence of SEQ ID NO: 413, and a CDR H3 having an amino acid sequence 100% identical to the amino acid sequence of SEQ ID NO: 414. In some embodiments, the antibody comprises a complementarity determining region (CDR) L1 having 100% sequence identity to an amino acid sequence of SEQ ID NO: 416, a CDR L2 having an amino acid sequence 100% identical to the amino acid sequence of DAF, and a CDR L3 having an amino acid sequence 100% identical to the amino acid sequence of SEQ ID NO: 417. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 411. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 415. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 411. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 415. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 411. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 415. In some embodiments, the antibody comprises a variable heavy chain (VH) region having an amino acid sequence with at least 95% (e.g., at least 95%, 96%, 97%, 98%, 99%, or more) identity to SEQ ID NO: 411. In some embodiments, the antibody comprises a variable light chain (VL) region having an amino acid sequence with at least 95% (e.g., at least 95%, 96%, 97%, 98%, 99%, or more) identity to SEQ ID NO: 415. In some embodiments, the antibody comprises a variable heavy chain (VH) region having an amino acid sequence 100% identical with the amino acid sequence of SEQ ID NO: 411. In some embodiments, the antibody comprises a variable light chain (VL) region having an amino acid sequence 100% identical with the amino acid sequence of SEQ ID NO: 415. In some embodiments, the antibody comprises: (a) a complementarity determining region (CDR) H1 having an amino acid sequence 100% identical to the amino acid sequence of SEQ ID NO: 412, a CDR H2 having an amino acid sequence 100% identical to the amino acid sequence of SEQ ID NO: 413, and a CDR H3 having an amino acid sequence 100% identical to the amino acid sequence of SEQ ID NO: 414; and (b) a complementarity determining region (CDR) L1 having 100% sequence identity to an amino acid sequence of SEQ ID NO: 416, a CDR L2 having an amino acid sequence 100% identical to the amino acid sequence of DAF, and a CDR L3 having an amino acid sequence 100% identical to the amino acid sequence of SEQ ID NO: 417. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 411 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 415. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 411 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 415. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 411 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 415. In some embodiments, the antibody comprises a variable heavy chain (VH) region having an amino acid sequence with at least 95% (e.g., at least 95%, 96%, 97%, 98%, 99%, or more) identity to SEQ ID NO: 411 and a variable light chain (VL) region having an amino acid sequence with at least 95% (e.g., at least 95%, 96%, 97%, 98%, 99%, or more) identity to SEQ ID NO: 415. In some embodiments, the antibody comprises a variable heavy chain (VH) region having an amino acid sequence 100% identical with the amino acid sequence of SEQ ID NO: 411 and a variable light chain (VL) region having an amino acid sequence 100% identical with the amino acid sequence of SEQ ID NO: 415. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 411 and comprising therein a complementarity determining region (CDR) H1 having an amino acid sequence 100% identical to the amino acid sequence of SEQ ID NO: 412, a CDR H2 having an amino acid sequence 100% identical to the amino acid sequence of SEQ ID NO: 413, and a CDR H3 having an amino acid sequence 100% identical to the amino acid sequence of SEQ ID NO: 414. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 415 and comprising therein a complementarity determining region (CDR) L1 having 100% sequence identity to an amino acid sequence of SEQ ID NO: 416, a CDR L2 having an amino acid sequence 100% identical to the amino acid sequence of DAF, and a CDR L3 having an amino acid sequence 100% identical to the amino acid sequence of SEQ ID NO: 417. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 411 and comprising therein a complementarity determining region (CDR) H1 having an amino acid sequence 100% identical to the amino acid sequence of SEQ ID NO: 412, a CDR H2 having an amino acid sequence 100% identical to the amino acid sequence of SEQ ID NO: 413, and a CDR H3 having an amino acid sequence 100% identical to the amino acid sequence of SEQ ID NO: 414; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 415 and comprising therein a complementarity determining region (CDR) L1 having 100% sequence identity to an amino acid sequence of SEQ ID NO: 416, a CDR L2 having an amino acid sequence 100% identical to the amino acid sequence of DAF, and a CDR L3 having an amino acid sequence 100% identical to the amino acid sequence of SEQ ID NO: 417.

In some embodiments, the antibody comprises a complementarity determining region (CDR) H1 having an amino acid sequence 100% identical to the amino acid sequence of SEQ ID NO: 419, a CDR H2 having an amino acid sequence 100% identical to the amino acid sequence of SEQ ID NO: 420, and a CDR H3 having an amino acid sequence 100% identical to the amino acid sequence of SEQ ID NO: 421. In some embodiments, the antibody comprises a complementarity determining region (CDR) L1 having 100% sequence identity to an amino acid sequence of SEQ ID NO: 423, a CDR L2 having an amino acid sequence 100% identical to the amino acid sequence of DAS, and a CDR L3 having an amino acid sequence 100% identical to the amino acid sequence of SEQ ID NO: 424. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 418. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 422. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 418. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 422. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 418. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 422. In some embodiments, the antibody comprises a variable heavy chain (VH) region having an amino acid sequence with at least 95% (e.g., at least 95%, 96%, 97%, 98%, 99%, or more) identity to SEQ ID NO: 418. In some embodiments, the antibody comprises a variable light chain (VL) region having an amino acid sequence with at least 95% (e.g., at least 95%, 96%, 97%, 98%, 99%, or more) identity to SEQ ID NO: 422. In some embodiments, the antibody comprises a variable heavy chain (VH) region having an amino acid sequence 100% identical with the amino acid sequence of SEQ ID NO: 418. In some embodiments, the antibody comprises a variable light chain (VL) region having an amino acid sequence 100% identical with the amino acid sequence of SEQ ID NO: 422. In some embodiments, the antibody comprises: (a) a complementarity determining region (CDR) H1 having an amino acid sequence 100% identical to the amino acid sequence of SEQ ID NO: 419, a CDR H2 having an amino acid sequence 100% identical to the amino acid sequence of SEQ ID NO: 420, and a CDR H3 having an amino acid sequence 100% identical to the amino acid sequence of SEQ ID NO: 421; and (b) a complementarity determining region (CDR) L1 having 100% sequence identity to an amino acid sequence of SEQ ID NO: 423, a CDR L2 having an amino acid sequence 100% identical to the amino acid sequence of DAS, and a CDR L3 having an amino acid sequence 100% identical to the amino acid sequence of SEQ ID NO: 424. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 418 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 422. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 418 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 422. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 418 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 422. In some embodiments, the antibody comprises a variable heavy chain (VH) region having an amino acid sequence with at least 95% (e.g., at least 95%, 96%, 97%, 98%, 99%, or more) identity to SEQ ID NO: 418 and a variable light chain (VL) region having an amino acid sequence with at least 95% (e.g., at least 95%, 96%, 97%, 98%, 99%, or more) identity to SEQ ID NO: 422. In some embodiments, the antibody comprises a variable heavy chain (VH) region having an amino acid sequence 100% identical with the amino acid sequence of SEQ ID NO: 418 and a variable light chain (VL) region having an amino acid sequence 100% identical with the amino acid sequence of SEQ ID NO: 422. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 418 and comprising therein a complementarity determining region (CDR) H1 having an amino acid sequence 100% identical to the amino acid sequence of SEQ ID NO: 419, a CDR H2 having an amino acid sequence 100% identical to the amino acid sequence of SEQ ID NO: 420, and a CDR H3 having an amino acid sequence 100% identical to the amino acid sequence of SEQ ID NO: 421. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 422 and comprising therein a complementarity determining region (CDR) L1 having 100% sequence identity to an amino acid sequence of SEQ ID NO: 423, a CDR L2 having an amino acid sequence 100% identical to the amino acid sequence of DAS, and a CDR L3 having an amino acid sequence 100% identical to the amino acid sequence of SEQ ID NO: 424. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 418 and comprising therein a complementarity determining region (CDR) H1 having an amino acid sequence 100% identical to the amino acid sequence of SEQ ID NO: 419, a CDR H2 having an amino acid sequence 100% identical to the amino acid sequence of SEQ ID NO: 420, and a CDR H3 having an amino acid sequence 100% identical to the amino acid sequence of SEQ ID NO: 421; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 422 and comprising therein a complementarity determining region (CDR) L1 having 100% sequence identity to an amino acid sequence of SEQ ID NO: 423, a CDR L2 having an amino acid sequence 100% identical to the amino acid sequence of DAS, and a CDR L3 having an amino acid sequence 100% identical to the amino acid sequence of SEQ ID NO: 424.

In some embodiments, the antibody comprises a complementarity determining region (CDR) H1 having an amino acid sequence 100% identical to the amino acid sequence of SEQ ID NO: 426, a CDR H2 having an amino acid sequence 100% identical to the amino acid sequence of SEQ ID NO: 427, and a CDR H3 having an amino acid sequence 100% identical to the amino acid sequence of SEQ ID NO: 428. In some embodiments, the antibody comprises a complementarity determining region (CDR) L1 having 100% sequence identity to an amino acid sequence of SEQ ID NO: 430, a CDR L2 having an amino acid sequence 100% identical to the amino acid sequence of DAS, and a CDR L3 having an amino acid sequence 100% identical to the amino acid sequence of SEQ ID NO: 431. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 425. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 429. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 425. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 429. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 425. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 429. In some embodiments, the antibody comprises a variable heavy chain (VH) region having an amino acid sequence with at least 95% (e.g., at least 95%, 96%, 97%, 98%, 99%, or more) identity to SEQ ID NO: 425. In some embodiments, the antibody comprises a variable light chain (VL) region having an amino acid sequence with at least 95% (e.g., at least 95%, 96%, 97%, 98%, 99%, or more) identity to SEQ ID NO: 429. In some embodiments, the antibody comprises a variable heavy chain (VH) region having an amino acid sequence 100% identical with the amino acid sequence of SEQ ID NO: 425. In some embodiments, the antibody comprises a variable light chain (VL) region having an amino acid sequence 100% identical with the amino acid sequence of SEQ ID NO: 429. In some embodiments, the antibody comprises: (a) a complementarity determining region (CDR) H1 having an amino acid sequence 100% identical to the amino acid sequence of SEQ ID NO: 426, a CDR H2 having an amino acid sequence 100% identical to the amino acid sequence of SEQ ID NO: 427, and a CDR H3 having an amino acid sequence 100% identical to the amino acid sequence of SEQ ID NO: 428; and (b) a complementarity determining region (CDR) L1 having 100% sequence identity to an amino acid sequence of SEQ ID NO: 430, a CDR L2 having an amino acid sequence 100% identical to the amino acid sequence of DAS, and a CDR L3 having an amino acid sequence 100% identical to the amino acid sequence of SEQ ID NO: 431. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 425 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 429. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 425 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 429. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 425 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 429. In some embodiments, the antibody comprises a variable heavy chain (VH) region having an amino acid sequence with at least 95% (e.g., at least 95%, 96%, 97%, 98%, 99%, or more) identity to SEQ ID NO: 425 and a variable light chain (VL) region having an amino acid sequence with at least 95% (e.g., at least 95%, 96%, 97%, 98%, 99%, or more) identity to SEQ ID NO: 429. In some embodiments, the antibody comprises a variable heavy chain (VH) region having an amino acid sequence 100% identical with the amino acid sequence of SEQ ID NO: 425 and a variable light chain (VL) region having an amino acid sequence 100% identical with the amino acid sequence of SEQ ID NO: 429. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 425 and comprising therein a complementarity determining region (CDR) H1 having an amino acid sequence 100% identical to the amino acid sequence of SEQ ID NO: 426, a CDR H2 having an amino acid sequence 100% identical to the amino acid sequence of SEQ ID NO: 427, and a CDR H3 having an amino acid sequence 100% identical to the amino acid sequence of SEQ ID NO: 428. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 429 and comprising therein a complementarity determining region (CDR) L1 having 100% sequence identity to an amino acid sequence of SEQ ID NO: 430, a CDR L2 having an amino acid sequence 100% identical to the amino acid sequence of DAS, and a CDR L3 having an amino acid sequence 100% identical to the amino acid sequence of SEQ ID NO: 431. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 425 and comprising therein a complementarity determining region (CDR) H1 having an amino acid sequence 100% identical to the amino acid sequence of SEQ ID NO: 426, a CDR H2 having an amino acid sequence 100% identical to the amino acid sequence of SEQ ID NO: 427, and a CDR H3 having an amino acid sequence 100% identical to the amino acid sequence of SEQ ID NO: 428; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 429 and comprising therein a complementarity determining region (CDR) L1 having 100% sequence identity to an amino acid sequence of SEQ ID NO: 430, a CDR L2 having an amino acid sequence 100% identical to the amino acid sequence of DAS, and a CDR L3 having an amino acid sequence 100% identical to the amino acid sequence of SEQ ID NO: 431.

In some embodiments, the antibody comprises a complementarity determining region (CDR) H1 having an amino acid sequence 100% identical to the amino acid sequence of SEQ ID NO: 433, a CDR H2 having an amino acid sequence 100% identical to the amino acid sequence of SEQ ID NO: 434, and a CDR H3 having an amino acid sequence 100% identical to the amino acid sequence of SEQ ID NO: 435. In some embodiments, the antibody comprises a complementarity determining region (CDR) L1 having 100% sequence identity to an amino acid sequence of SEQ ID NO: 437, a CDR L2 having an amino acid sequence 100% identical to the amino acid sequence of GAS, and a CDR L3 having an amino acid sequence 100% identical to the amino acid sequence of SEQ ID NO: 438. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 432. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 436. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 432. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 436. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 432. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 436. In some embodiments, the antibody comprises a variable heavy chain (VH) region having an amino acid sequence with at least 95% (e.g., at least 95%, 96%, 97%, 98%, 99%, or more) identity to SEQ ID NO: 432. In some embodiments, the antibody comprises a variable light chain (VL) region having an amino acid sequence with at least 95% (e.g., at least 95%, 96%, 97%, 98%, 99%, or more) identity to SEQ ID NO: 436. In some embodiments, the antibody comprises a variable heavy chain (VH) region having an amino acid sequence 100% identical with the amino acid sequence of SEQ ID NO: 432. In some embodiments, the antibody comprises a variable light chain (VL) region having an amino acid sequence 100% identical with the amino acid sequence of SEQ ID NO: 436. In some embodiments, the antibody comprises: (a) a complementarity determining region (CDR) H1 having an amino acid sequence 100% identical to the amino acid sequence of SEQ ID NO: 433, a CDR H2 having an amino acid sequence 100% identical to the amino acid sequence of SEQ ID NO: 434, and a CDR H3 having an amino acid sequence 100% identical to the amino acid sequence of SEQ ID NO: 435; and (b) a complementarity determining region (CDR) L1 having 100% sequence identity to an amino acid sequence of SEQ ID NO: 437, a CDR L2 having an amino acid sequence 100% identical to the amino acid sequence of GAS, and a CDR L3 having an amino acid sequence 100% identical to the amino acid sequence of SEQ ID NO: 438. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 432 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 436. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 432 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 436. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 432 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 436. In some embodiments, the antibody comprises a variable heavy chain (VH) region having an amino acid sequence with at least 95% (e.g., at least 95%, 96%, 97%, 98%, 99%, or more) identity to SEQ ID NO: 432 and a variable light chain (VL) region having an amino acid sequence with at least 95% (e.g., at least 95%, 96%, 97%, 98%, 99%, or more) identity to SEQ ID NO: 436. In some embodiments, the antibody comprises a variable heavy chain (VH) region having an amino acid sequence 100% identical with the amino acid sequence of SEQ ID NO: 432 and a variable light chain (VL) region having an amino acid sequence 100% identical with the amino acid sequence of SEQ ID NO: 436. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 432 and comprising therein a complementarity determining region (CDR) H1 having an amino acid sequence 100% identical to the amino acid sequence of SEQ ID NO: 433, a CDR H2 having an amino acid sequence 100% identical to the amino acid sequence of SEQ ID NO: 434, and a CDR H3 having an amino acid sequence 100% identical to the amino acid sequence of SEQ ID NO: 435. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 436 and comprising therein a complementarity determining region (CDR) L1 having 100% sequence identity to an amino acid sequence of SEQ ID NO: 437, a CDR L2 having an amino acid sequence 100% identical to the amino acid sequence of GAS, and a CDR L3 having an amino acid sequence 100% identical to the amino acid sequence of SEQ ID NO: 438. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 432 and comprising therein a complementarity determining region (CDR) H1 having an amino acid sequence 100% identical to the amino acid sequence of SEQ ID NO: 433, a CDR H2 having an amino acid sequence 100% identical to the amino acid sequence of SEQ ID NO: 434, and a CDR H3 having an amino acid sequence 100% identical to the amino acid sequence of SEQ ID NO: 435; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 436 and comprising therein a complementarity determining region (CDR) L1 having 100% sequence identity to an amino acid sequence of SEQ ID NO: 437, a CDR L2 having an amino acid sequence 100% identical to the amino acid sequence of GAS, and a CDR L3 having an amino acid sequence 100% identical to the amino acid sequence of SEQ ID NO: 438.

In some embodiments, the antibody comprises a complementarity determining region (CDR) H1 having an amino acid sequence 100% identical to the amino acid sequence of SEQ ID NO: 440, a CDR H2 having an amino acid sequence 100% identical to the amino acid sequence of SEQ ID NO: 441, and a CDR H3 having an amino acid sequence 100% identical to the amino acid sequence of SEQ ID NO: 442. In some embodiments, the antibody comprises a complementarity determining region (CDR) L1 having 100% sequence identity to an amino acid sequence of SEQ ID NO: 444, a CDR L2 having an amino acid sequence 100% identical to the amino acid sequence of AAS, and a CDR L3 having an amino acid sequence 100% identical to the amino acid sequence of SEQ ID NO: 445. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 439. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 443. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 439. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 443. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 439. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 443. In some embodiments, the antibody comprises a variable heavy chain (VH) region having an amino acid sequence with at least 95% (e.g., at least 95%, 96%, 97%, 98%, 99%, or more) identity to SEQ ID NO: 439. In some embodiments, the antibody comprises a variable light chain (VL) region having an amino acid sequence with at least 95% (e.g., at least 95%, 96%, 97%, 98%, 99%, or more) identity to SEQ ID NO: 443. In some embodiments, the antibody comprises a variable heavy chain (VH) region having an amino acid sequence 100% identical with the amino acid sequence of SEQ ID NO: 439. In some embodiments, the antibody comprises a variable light chain (VL) region having an amino acid sequence 100% identical with the amino acid sequence of SEQ ID NO: 443. In some embodiments, the antibody comprises: (a) a complementarity determining region (CDR) H1 having an amino acid sequence 100% identical to the amino acid sequence of SEQ ID NO: 440, a CDR H2 having an amino acid sequence 100% identical to the amino acid sequence of SEQ ID NO: 441, and a CDR H3 having an amino acid sequence 100% identical to the amino acid sequence of SEQ ID NO: 442; and (b) a complementarity determining region (CDR) L1 having 100% sequence identity to an amino acid sequence of SEQ ID NO: 444, a CDR L2 having an amino acid sequence 100% identical to the amino acid sequence of AAS, and a CDR L3 having an amino acid sequence 100% identical to the amino acid sequence of SEQ ID NO: 445. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 439 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 443. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 439 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 443. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 439 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 443. In some embodiments, the antibody comprises a variable heavy chain (VH) region having an amino acid sequence with at least 95% (e.g., at least 95%, 96%, 97%, 98%, 99%, or more) identity to SEQ ID NO: 439 and a variable light chain (VL) region having an amino acid sequence with at least 95% (e.g., at least 95%, 96%, 97%, 98%, 99%, or more) identity to SEQ ID NO: 443. In some embodiments, the antibody comprises a variable heavy chain (VH) region having an amino acid sequence 100% identical with the amino acid sequence of SEQ ID NO: 439 and a variable light chain (VL) region having an amino acid sequence 100% identical with the amino acid sequence of SEQ ID NO: 443. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 439 and comprising therein a complementarity determining region (CDR) H1 having an amino acid sequence 100% identical to the amino acid sequence of SEQ ID NO: 440, a CDR H2 having an amino acid sequence 100% identical to the amino acid sequence of SEQ ID NO: 441, and a CDR H3 having an amino acid sequence 100% identical to the amino acid sequence of SEQ ID NO: 442. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 443 and comprising therein a complementarity determining region (CDR) L1 having 100% sequence identity to an amino acid sequence of SEQ ID NO: 444, a CDR L2 having an amino acid sequence 100% identical to the amino acid sequence of AAS, and a CDR L3 having an amino acid sequence 100% identical to the amino acid sequence of SEQ ID NO: 445. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 439 and comprising therein a complementarity determining region (CDR) H1 having an amino acid sequence 100% identical to the amino acid sequence SEQ ID NO: 440, a CDR H2 having an amino acid sequence 100% identical to the amino acid sequence of SEQ ID NO: 441, and a CDR H3 having an amino acid sequence 100% identical to the amino acid sequence of SEQ ID NO: 442; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 443 and comprising therein a complementarity determining region (CDR) L1 having 100% sequence identity to an amino acid sequence of SEQ ID NO: 444, a CDR L2 having an amino acid sequence 100% identical to the amino acid sequence of AAS, and a CDR L3 having an amino acid sequence 100% identical to the amino acid sequence of SEQ ID NO: 445.

In some embodiments, the antibody comprises a complementarity determining region (CDR) H1 having an amino acid sequence 100% identical to the amino acid sequence of SEQ ID NO: 447, a CDR H2 having an amino acid sequence 100% identical to the amino acid sequence of SEQ ID NO: 448, and a CDR H3 having an amino acid sequence 100% identical to the amino acid sequence of SEQ ID NO: 449. In some embodiments, the antibody comprises a complementarity determining region (CDR) L1 having 100% sequence identity to an amino acid sequence of SEQ ID NO: 451, a CDR L2 having an amino acid sequence 100% identical to the amino acid sequence of DNF, and a CDR L3 having an amino acid sequence 100% identical to the amino acid sequence of SEQ ID NO: 452. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 446. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 450. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 446. In some embodiments, the antibody comprises a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 450. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 446. In some embodiments, the antibody comprises a variable light chain (VL) region having an amino acid sequence with at least 90% (e.g., at least 90%, 91%, 92%, 93%, 94%, 95%, 96%, 97%, 98%, 99%, or more) identity to SEQ ID NO: 450. In some embodiments, the antibody comprises a variable heavy chain (VH) region having an amino acid sequence with at least 95% (e.g., at least 95%, 96%, 97%, 98%, 99%, or more) identity to SEQ ID NO: 446. In some embodiments, the antibody comprises a variable light chain (VL) region having an amino acid sequence with at least 95% (e.g., at least 95%, 96%, 97%, 98%, 99%, or more) identity to SEQ ID NO: 450. In some embodiments, the antibody comprises a variable heavy chain (VH) region having an amino acid sequence 100% identical with the amino acid sequence of SEQ ID NO: 446. In some embodiments, the antibody comprises a variable light chain (VL) region having an amino acid sequence 100% identical with the amino acid sequence of SEQ ID NO: 450. In some embodiments, the antibody comprises: (a) a complementarity determining region (CDR) H1 having an amino acid sequence 100% identical to the amino acid sequence of SEQ ID NO: 447, a CDR H2 having an amino acid sequence 100% identical to the amino acid sequence of SEQ ID NO: 448, and a CDR H3 having an amino acid sequence 100% identical to the amino acid sequence of SEQ ID NO: 449; and (b) a complementarity determining region (CDR) L1 having 100% sequence identity to an amino acid sequence of SEQ ID NO: 451, a CDR L2 having an amino acid sequence 100% identical to the amino acid sequence of DNF, and a CDR L3 having an amino acid sequence 100% identical to the amino acid sequence of SEQ ID NO: 452. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 446 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 450. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 446 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 450. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 446 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 450. In some embodiments, the antibody comprises a variable heavy chain (VH) region having an amino acid sequence with at least 95% (e.g., at least 95%, 96%, 97%, 98%, 99%, or more) identity to SEQ ID NO: 446 and a variable light chain (VL) region having an amino acid sequence with at least 95% (e.g., at least 95%, 96%, 97%, 98%, 99%, or more) identity to SEQ ID NO: 450. In some embodiments, the antibody comprises a variable heavy chain (VH) region having an amino acid sequence 100% identical with the amino acid sequence of SEQ ID NO: 446 and a variable light chain (VL) region having an amino acid sequence 100% identical with the amino acid sequence of SEQ ID NO: 450. In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 446 and comprising therein a complementarity determining region (CDR) H1 having an amino acid sequence 100% identical to the amino acid sequence of SEQ ID NO: 447, a CDR H2 having an amino acid sequence 100% identical to the amino acid sequence of SEQ ID NO: 448, and a CDR H3 having an amino acid sequence 100% identical to the amino acid sequence of SEQ ID NO: 449. In some embodiments, the antibody comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 450 and comprising therein a complementarity determining region (CDR) L1 having 100% sequence identity to an amino acid sequence of SEQ ID NO: 451, a CDR L2 having an amino acid sequence 100% identical to the amino acid sequence of DNF, and a CDR L3 having an amino acid sequence 100% identical to the amino acid sequence of SEQ ID NO: 452. In some embodiments, the antibody comprises: (a) a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 446 and comprising therein a complementarity determining region (CDR) H1 having an amino acid sequence 100% identical to the amino acid sequence of SEQ ID NO: 447, a CDR H2 having an amino acid sequence 100% identical to the amino acid sequence of SEQ ID NO: 448, and a CDR H3 having an amino acid sequence 100% identical to the amino acid sequence of SEQ ID NO: 449; and (b) a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 450 and comprising therein a complementarity determining region (CDR) L1 having 100% sequence identity to an amino acid sequence of SEQ ID NO: 451, a CDR L2 having an amino acid sequence 100% identical to the amino acid sequence of DNF, and a CDR L3 having an amino acid sequence 100% identical to the amino acid sequence of SEQ ID NO: 452.

In some embodiments, an antibody disclosed herein comprises a heavy chain (HC) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 453 and a light chain (LC) region having at least 80% sequence identity to any one of the LC (“VL_LC”) regions listed in Table 4.

In some embodiments, an antibody disclosed herein comprises a heavy chain (HC) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 454 and a light chain (LC) region having at least 80% sequence identity to any one of the LC (“VL_LC”) regions listed in Table 4.

In some embodiments, an antibody disclosed herein comprises a heavy chain (HC) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 455 and a light chain (LC) region having at least 80% sequence identity to any one of the LC (“VL_LC”) regions listed in Table 4.

In some embodiments, an antibody disclosed herein comprises a heavy chain (HC) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 456 and a light chain (LC) region having at least 80% sequence identity to any one of the LC (“VL_LC”) regions listed in Table 4.

In some embodiments, an antibody disclosed herein comprises a heavy chain (HC) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 457 and a light chain (LC) region having at least 80% sequence identity to any one of the LC (“VL_LC”) regions listed in Table 4.

In some embodiments, an antibody disclosed herein comprises a heavy chain (HC) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 458 and a light chain (LC) region having at least 80% sequence identity to any one of the LC (“VL_LC”) regions listed in Table 4.

In some embodiments, an antibody disclosed herein comprises a heavy chain (HC) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 459 and a light chain (LC) region having at least 80% sequence identity to any one of the LC (“VL_LC”) regions listed in Table 4.

In some embodiments, an antibody disclosed herein comprises a heavy chain (HC) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 460 and a light chain (LC) region having at least 80% sequence identity to any one of the LC (“VL_LC”) regions listed in Table 4.

In some embodiments, an antibody disclosed herein comprises a heavy chain (HC) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 461 and a light chain (LC) region having at least 80% sequence identity to any one of the LC (“VL_LC”) regions listed in Table 4.

In some embodiments, an antibody disclosed herein comprises a heavy chain (HC) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 462 and a light chain (LC) region having at least 80% sequence identity to any one of the LC (“VL_LC”) regions listed in Table 4.

In some embodiments, an antibody disclosed herein comprises a light chain (LC) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 463 and a heavy chain (HC) region having at least 80% sequence identity to any one of the HC (“VH_HC”) regions listed in Table 4.

In some embodiments, an antibody disclosed herein comprises a light chain (LC) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 464 and a heavy chain (HC) region having at least 80% sequence identity to any one of the HC (“VH_HC”) regions listed in Table 4.

In some embodiments, an antibody disclosed herein comprises a light chain (LC) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 465 and a heavy chain (HC) region having at least 80% sequence identity to any one of the HC (“VH_HC”) regions listed in Table 4.

In some embodiments, an antibody disclosed herein comprises a light chain (LC) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 466 and a heavy chain (HC) region having at least 80% sequence identity to any one of the HC (“VH_HC”) regions listed in Table 4.

In some embodiments, an antibody disclosed herein comprises a light chain (LC) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 467 and a heavy chain (HC) region having at least 80% sequence identity to any one of the HC (“VH_HC”) regions listed in Table 4.

In some embodiments, an antibody disclosed herein comprises a light chain (LC) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 468 and a heavy chain (HC) region having at least 80% sequence identity to any one of the HC (“VH_HC”) regions listed in Table 4.

In some embodiments, an antibody disclosed herein comprises a light chain (LC) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 469 and a heavy chain (HC) region having at least 80% sequence identity to any one of the HC (“VH_HC”) regions listed in Table 4.

In some embodiments, an antibody disclosed herein comprises a light chain (LC) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 470] and a heavy chain (HC) region having at least 80% sequence identity to any one of the HC (“VH_HC”) regions listed in Table 4.

In some embodiments, an antibody disclosed herein comprises a light chain (LC) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 471 and a heavy chain (HC) region having at least 80% sequence identity to any one of the HC (“VH_HC”) regions listed in Table 4.

In some embodiments, an antibody disclosed herein comprises a light chain (LC) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%9, 97%, 98%, 99%, or more) identity to SEQ ID NO: 472 and a heavy chain (HC) region having at least 80% sequence identity to any one of the HC (“VH_HC”) regions listed in Table 4.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 12 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 19 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 26 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 33 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 40 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 47 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 54 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 61 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 68 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 75 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 82 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 89 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 96 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 103 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 110 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 117 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 124 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 131 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 138 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 145 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 152 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 159 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 166 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 173 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 180 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 187 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 194 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 201 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 208 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 215 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 222 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 229 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 236 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 243 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 250 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 257 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 264 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 271 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 278 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 285 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 292 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 299 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 306 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 313 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 320 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 327 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 334 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 341 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 348 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 355 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 362 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 369 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 376 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 383 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 390 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 397 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 404 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 411 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 418 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 425 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 432 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 439 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 446 and a variable light chain (VL) region having at least 80% sequence identity to any one of the VL regions listed in Table 1.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 446 and a variable light chain (VL) region having at least 80% sequence identity to any one of the VL regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 12 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 23 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 30 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 37 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 44 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 51 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 58 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 65 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 72 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 79 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 86 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 93 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 100 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 107 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 114 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 121 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 128 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 135 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 142 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 149 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 156 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 163 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 170 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 177 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 184 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 191 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 198 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 205 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 212 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 219 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 226 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 233 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 240 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 247 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 254 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 261 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 268 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 275 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 282 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 289 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 296 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 303 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 310 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 317 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 324 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 331 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 338 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 345 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 352 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 359 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 366 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 373 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 380 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 387 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 394 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 401 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 408 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 415 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 422 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 429 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 436 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 443 and a variable heavy chain (VH) region having at least 80% sequence identity to any one of the VH regions listed in Table 1.

In some embodiments, an antibody disclosed herein comprises a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 450 and a variable heavy chain (VH) region having at least 80% sequence identity to any one of the VH regions listed in Table 1.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 13, a CDR H2 having an amino acid sequence 100% identical to the amino acid sequence of SEQ ID NO: 14, and a CDR H3 having an amino acid sequence 100% identical to the amino acid sequence of SEQ ID NO: 15; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 17, a CDR L2 having an amino acid sequence 100% identical to the amino acid sequence of KAS, and a CDR L3 having an amino acid sequence 100% identical to the amino acid sequence of SEQ ID NO: 18.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 20, a CDR H2 having an amino acid sequence 100% identical to the amino acid sequence of SEQ ID NO: 21, and a CDR H3 having an amino acid sequence 100% identical to the amino acid sequence of SEQ ID NO: 22; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 24, a CDR L2 having an amino acid sequence 100% identical to the amino acid sequence of AAS, and a CDR L3 having an amino acid sequence 100% identical to the amino acid sequence of SEQ ID NO: 25.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 27, a CDR H2 having an amino acid sequence 100% identical to the amino acid sequence of SEQ ID NO: 28, and a CDR H3 having an amino acid sequence 100% identical to the amino acid sequence of SEQ ID NO: 29; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 31, a CDR L2 having an amino acid sequence 100% identical to the amino acid sequence of ESS, and a CDR L3 having an amino acid sequence 100% identical to the amino acid sequence of SEQ ID NO: 32.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 34, a CDR H2 having an amino acid sequence 100% identical to the amino acid sequence of SEQ ID NO: 35, and a CDR H3 having an amino acid sequence 100% identical to the amino acid sequence of SEQ ID NO: 36; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 38, a CDR L2 having an amino acid sequence 100% identical to the amino acid sequence of DAT, and a CDR L3 having an amino acid sequence 100% identical to the amino acid sequence of SEQ ID NO: 39.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 41, a CDR H2 having an amino acid sequence 100% identical to the amino acid sequence of SEQ ID NO: 42, and a CDR H3 having an amino acid sequence 100% identical to the amino acid sequence of SEQ ID NO: 43; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 45, a CDR L2 having an amino acid sequence 100% identical to the amino acid sequence of SEQ ID NO: GSS, and a CDR L3 having an amino acid sequence 100% identical to the amino acid sequence of SEQ ID NO: 46.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 48, a CDR H2 having an amino acid sequence 100% identical to the amino acid sequence of SEQ ID NO: 49, and a CDR H3 having an amino acid sequence 100% identical to the amino acid sequence of SEQ ID NO: 50; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 52, a CDR L2 having an amino acid sequence 100% identical to the amino acid sequence of DAT, and a CDR L3 having an amino acid sequence 100% identical to the amino acid sequence of SEQ ID NO: 53.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 55, a CDR H2 having an amino acid sequence 100% identical to the amino acid sequence of SEQ ID NO: 56, and a CDR H3 having an amino acid sequence 100% identical to the amino acid sequence of SEQ ID NO: 57; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 59, a CDR L2 having an amino acid sequence 100% identical to the amino acid sequence of GAS, and a CDR L3 having an amino acid sequence 100% identical to the amino acid sequence of SEQ ID NO: 60.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 62, a CDR H2 having an amino acid sequence 100% identical to the amino acid sequence of SEQ ID NO: 63, and a CDR H3 having an amino acid sequence 100% identical to the amino acid sequence of SEQ ID NO: 64; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 66, a CDR L2 having an amino acid sequence 100% identical to the amino acid sequence of DAS, and a CDR L3 having an amino acid sequence 100% identical to the amino acid sequence of SEQ ID NO: 67.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 69, a CDR H2 having an amino acid sequence 100% identical to the amino acid sequence of SEQ ID NO: 70, and a CDR H3 having an amino acid sequence 100% identical to the amino acid sequence of SEQ ID NO: 71; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 73, a CDR L2 having an amino acid sequence 100% identical to the amino acid sequence of GAS, and a CDR L3 having an amino acid sequence 100% identical to the amino acid sequence of SEQ ID NO: 74.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 76, a CDR H2 having an amino acid sequence 100% identical to the amino acid sequence of SEQ ID NO: 77, and a CDR H3 having an amino acid sequence 100% identical to the amino acid sequence of SEQ ID NO: 78; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 80, a CDR L2 having an amino acid sequence 100% identical to the amino acid sequence of SEQ ID NO: HAS, and a CDR L3 having an amino acid sequence 100% identical to the amino acid sequence of SEQ ID NO: 81.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 83, a CDR H2 having an amino acid sequence 100% identical to the amino acid sequence of SEQ ID NO: 84, and a CDR H3 having an amino acid sequence 100% identical to the amino acid sequence of SEQ ID NO: 85; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 87, a CDR L2 having an amino acid sequence 100% identical to the amino acid sequence of DAT, and a CDR L3 having an amino acid sequence 100% identical to the amino acid sequence of SEQ ID NO: 88.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 90, a CDR H2 having an amino acid sequence 100% identical to the amino acid sequence of SEQ ID NO: 91, and a CDR H3 having an amino acid sequence 100% identical to the amino acid sequence of SEQ ID NO: 92; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 94, a CDR L2 having an amino acid sequence 100% identical to the amino acid sequence of GAS, and a CDR L3 having an amino acid sequence 100% identical to the amino acid sequence of SEQ ID NO: 95.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 97, a CDR H2 having an amino acid sequence 100% identical to the amino acid sequence of SEQ ID NO: 98, and a CDR H3 having an amino acid sequence 100% identical to the amino acid sequence of SEQ ID NO: 99; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO:101, a CDR L2 having an amino acid sequence 100% identical to the amino acid sequence of DAS, and a CDR L3 having an amino acid sequence 100% identical to the amino acid sequence of SEQ ID NO: 102.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 104, a CDR H2 having an amino acid sequence 100% identical to the amino acid sequence of SEQ ID NO: 105, and a CDR H3 having an amino acid sequence 100% identical to the amino acid sequence of SEQ ID NO: 106; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 108, a CDR L2 having an amino acid sequence 100% identical to the amino acid sequence of DIS, and a CDR L3 having an amino acid sequence 100% identical to the amino acid sequence of SEQ ID NO: 109.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 111, a CDR H2 having an amino acid sequence 100% identical to the amino acid sequence of SEQ ID NO: 112, and a CDR H3 having an amino acid sequence 100% identical to the amino acid sequence of SEQ ID NO: 113; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 115, a CDR L2 having an amino acid sequence 100% identical to the amino acid sequence of SEQ ID NO: DAS, and a CDR L3 having an amino acid sequence 100% identical to the amino acid sequence of SEQ ID NO: 116.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 118, a CDR H2 having an amino acid sequence 100% identical to the amino acid sequence of SEQ ID NO: 119, and a CDR H3 having an amino acid sequence 100% identical to the amino acid sequence of SEQ ID NO: 120; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 122, a CDR L2 having an amino acid sequence 100% identical to the amino acid sequence of DAS, and a CDR L3 having an amino acid sequence 100% identical to the amino acid sequence of SEQ ID NO: 123.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 125, a CDR H2 having an amino acid sequence 100% identical to the amino acid sequence of SEQ ID NO: 126, and a CDR H3 having an amino acid sequence 100% identical to the amino acid sequence of SEQ ID NO: 127; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 129, a CDR L2 having an amino acid sequence 100% identical to the amino acid sequence of EAS, and a CDR L3 having an amino acid sequence 100% identical to the amino acid sequence of SEQ ID NO: 130.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 132, a CDR H2 having an amino acid sequence 100% identical to the amino acid sequence of SEQ ID NO: 133, and a CDR H3 having an amino acid sequence 100% identical to the amino acid sequence of SEQ ID NO: 134; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 136, a CDR L2 having an amino acid sequence 100% identical to the amino acid sequence of DAS, and a CDR L3 having an amino acid sequence 100% identical to the amino acid sequence of SEQ ID NO: 137.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 139, a CDR H2 having an amino acid sequence 100% identical to the amino acid sequence of SEQ ID NO: 140, and a CDR H3 having an amino acid sequence 100% identical to the amino acid sequence of SEQ ID NO: 141; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 143, a CDR L2 having an amino acid sequence 100% identical to the amino acid sequence DAS, and a CDR L3 having an amino acid sequence 100% identical to the amino acid sequence of SEQ ID NO: 144.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 146, a CDR H2 having an amino acid sequence 100% identical to the amino acid sequence of SEQ ID NO: 147, and a CDR H3 having an amino acid sequence 100% identical to the amino acid sequence of SEQ ID NO: 148; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 150, a CDR L2 having an amino acid sequence 100% identical to the amino acid sequence of DAS, and a CDR L3 having an amino acid sequence 100% identical to the amino acid sequence of SEQ ID NO: 151.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 153, a CDR H2 having an amino acid sequence 100% identical to the amino acid sequence of SEQ ID NO: 154, and a CDR H3 having an amino acid sequence 100% identical to the amino acid sequence of SEQ ID NO: 155; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 157, a CDR L2 having an amino acid sequence 100% identical to the amino acid sequence of DAS, and a CDR L3 having an amino acid sequence 100% identical to the amino acid sequence of SEQ ID NO: 158.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 160, a CDR H2 having an amino acid sequence 100% identical to the amino acid sequence of SEQ ID NO: 161, and a CDR H3 having an amino acid sequence 100% identical to the amino acid sequence of SEQ ID NO: 162; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 164, a CDR L2 having an amino acid sequence 100% identical to the amino acid sequence of DAS, and a CDR L3 having an amino acid sequence 100% identical to the amino acid sequence of SEQ ID NO: 165.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 167, a CDR H2 having an amino acid sequence 100% identical to the amino acid sequence of SEQ ID NO: 168, and a CDR H3 having an amino acid sequence 100% identical to the amino acid sequence of SEQ ID NO: 169; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 171, a CDR L2 having an amino acid sequence 100% identical to the amino acid sequence of GAS, and a CDR L3 having an amino acid sequence 100% identical to the amino acid sequence of SEQ ID NO:172.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 174, a CDR H2 having an amino acid sequence 100% identical to the amino acid sequence of SEQ ID NO: 175, and a CDR H3 having an amino acid sequence 100% identical to the amino acid sequence of SEQ ID NO: 176; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 178, a CDR L2 having an amino acid sequence 100% identical to the amino acid sequence of GAS, and a CDR L3 having an amino acid sequence 100% identical to the amino acid sequence of SEQ ID NO: 179.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 181, a CDR H2 having an amino acid sequence 100% identical to the amino acid sequence of SEQ ID NO: 182, and a CDR H3 having an amino acid sequence 100% identical to the amino acid sequence of SEQ ID NO: 183; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 185, a CDR L2 having an amino acid sequence 100% identical to the amino acid sequence of DVS, and a CDR L3 having an amino acid sequence 100% identical to the amino acid sequence of SEQ ID NO: 186.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 188, a CDR H2 having an amino acid sequence 100% identical to the amino acid sequence of SEQ ID NO: 189, and a CDR H3 having an amino acid sequence 100% identical to the amino acid sequence of SEQ ID NO: 190; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 192, a CDR L2 having an amino acid sequence 100% identical to the amino acid sequence of DAT, and a CDR L3 having an amino acid sequence 100% identical to the amino acid sequence of SEQ ID NO: 193.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 195, a CDR H2 having an amino acid sequence 100% identical to the amino acid sequence of SEQ ID NO: 196, and a CDR H3 having an amino acid sequence 100% identical to the amino acid sequence of SEQ ID NO: 197; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 199, a CDR L2 having an amino acid sequence 100% identical to the amino acid sequence of DAS, and a CDR L3 having an amino acid sequence 100% identical to the amino acid sequence of SEQ ID NO: 200.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 202, a CDR H2 having an amino acid sequence 100% identical to the amino acid sequence of SEQ ID NO: 203, and a CDR H3 having an amino acid sequence 100% identical to the amino acid sequence of SEQ ID NO: 204; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 206, a CDR L2 having an amino acid sequence 100% identical to the amino acid sequence of GVD, and a CDR L3 having an amino acid sequence 100% identical to the amino acid sequence of SEQ ID NO: 207.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 209, a CDR H2 having an amino acid sequence 100% identical to the amino acid sequence of SEQ ID NO: 210, and a CDR H3 having an amino acid sequence 100% identical to the amino acid sequence of SEQ ID NO: 211; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 213, a CDR L2 having an amino acid sequence 100% identical to the amino acid sequence of DAS, and a CDR L3 having an amino acid sequence 100% identical to the amino acid sequence of SEQ ID NO: 214.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 216, a CDR H2 having an amino acid sequence 100% identical to the amino acid sequence of SEQ ID NO: 217, and a CDR H3 having an amino acid sequence 100% identical to the amino acid sequence of SEQ ID NO: 218; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 220, a CDR L2 having an amino acid sequence 100% identical to the amino acid sequence of GAT, and a CDR L3 having an amino acid sequence 100% identical to the amino acid sequence of SEQ ID NO: 221.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 223, a CDR H2 having an amino acid sequence 100% identical to the amino acid sequence of SEQ ID NO: 224, and a CDR H3 having an amino acid sequence 100% identical to the amino acid sequence of SEQ ID NO: 225; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 227, a CDR L2 having an amino acid sequence 100% identical to the amino acid sequence of DAS, and a CDR L3 having an amino acid sequence 100% identical to the amino acid sequence of SEQ ID NO: 228.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 230, a CDR H2 having an amino acid sequence 100% identical to the amino acid sequence of SEQ ID NO: 231, and a CDR H3 having an amino acid sequence 100% identical to the amino acid sequence of SEQ ID NO: 232; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 234, a CDR L2 having an amino acid sequence 100% identical to the amino acid sequence of DAS, and a CDR L3 having an amino acid sequence 100% identical to the amino acid sequence of SEQ ID NO: 235.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 237, a CDR H2 having an amino acid sequence 100% identical to the amino acid sequence of SEQ ID NO: 238, and a CDR H3 having an amino acid sequence 100% identical to the amino acid sequence of SEQ ID NO: 239; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 241, a CDR L2 having an amino acid sequence 100% identical to the amino acid sequence of GAS, and a CDR L3 having an amino acid sequence 100% identical to the amino acid sequence of SEQ ID NO: 242.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 244, a CDR H2 having an amino acid sequence 100% identical to the amino acid sequence of SEQ ID NO: 245, and a CDR H3 having an amino acid sequence 100% identical to the amino acid sequence of SEQ ID NO: 246; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 248, a CDR L2 having an amino acid sequence 100% identical to the amino acid sequence of GAS, and a CDR L3 having an amino acid sequence 100% identical to the amino acid sequence of SEQ ID NO: 249.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 251, a CDR H2 having an amino acid sequence 100% identical to the amino acid sequence of SEQ ID NO: 252, and a CDR H3 having an amino acid sequence 100% identical to the amino acid sequence of SEQ ID NO: 253; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 255, a CDR L2 having an amino acid sequence 100% identical to the amino acid sequence of GAT, and a CDR L3 having an amino acid sequence 100% identical to the amino acid sequence of SEQ ID NO: 256.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 258, a CDR H2 having an amino acid sequence 100% identical to the amino acid sequence of SEQ ID NO: 259, and a CDR H3 having an amino acid sequence 100% identical to the amino acid sequence of SEQ ID NO: 260; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 262, a CDR L2 having an amino acid sequence 100% identical to the amino acid sequence of GAS, and a CDR L3 having an amino acid sequence 100% identical to the amino acid sequence of SEQ ID NO: 263.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 265, a CDR H2 having an amino acid sequence 100% identical to the amino acid sequence of SEQ ID NO: 266, and a CDR H3 having an amino acid sequence 100% identical to the amino acid sequence of SEQ ID NO: 267; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 269, a CDR L2 having an amino acid sequence 100% identical to the amino acid sequence of DAV, and a CDR L3 having an amino acid sequence 100% identical to the amino acid sequence of SEQ ID NO: 270.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 272, a CDR H2 having an amino acid sequence 100% identical to the amino acid sequence of SEQ ID NO: 273, and a CDR H3 having an amino acid sequence 100% identical to the amino acid sequence of SEQ ID NO: 274; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 276, a CDR L2 having an amino acid sequence 100% identical to the amino acid sequence of DAS, and a CDR L3 having an amino acid sequence 100% identical to the amino acid sequence of SEQ ID NO: 277.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 279, a CDR H2 having an amino acid sequence 100% identical to the amino acid sequence of SEQ ID NO: 280, and a CDR H3 having an amino acid sequence 100% identical to the amino acid sequence of SEQ ID NO: 281; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 283, a CDR L2 having an amino acid sequence 100% identical to the amino acid sequence of DAS, and a CDR L3 having an amino acid sequence 100% identical to the amino acid sequence of SEQ ID NO: 284.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 286, a CDR H2 having an amino acid sequence 100% identical to the amino acid sequence of SEQ ID NO: 287, and a CDR H3 having an amino acid sequence 100% identical to the amino acid sequence of SEQ ID NO: 288; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 290, a CDR L2 having an amino acid sequence 100% identical to the amino acid sequence of GAS, and a CDR L3 having an amino acid sequence 100% identical to the amino acid sequence of SEQ ID NO: 291.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 293, a CDR H2 having an amino acid sequence 100% identical to the amino acid sequence of SEQ ID NO: 294, and a CDR H3 having an amino acid sequence 100% identical to the amino acid sequence of SEQ ID NO: 295; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 297, a CDR L2 having an amino acid sequence 100% identical to the amino acid sequence of KAS, and a CDR L3 having an amino acid sequence 100% identical to the amino acid sequence of SEQ ID NO: 298.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 300, a CDR H2 having an amino acid sequence 100% identical to the amino acid sequence of SEQ ID NO: 301, and a CDR H3 having an amino acid sequence 100% identical to the amino acid sequence of SEQ ID NO: 302; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 304, a CDR L2 having an amino acid sequence 100% identical to the amino acid sequence of AAS, and a CDR L3 having an amino acid sequence 100% identical to the amino acid sequence of SEQ ID NO: 305.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 307, a CDR H2 having an amino acid sequence 100% identical to the amino acid sequence of SEQ ID NO: 308, and a CDR H3 having an amino acid sequence 100% identical to the amino acid sequence of SEQ ID NO: 309; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 311, a CDR L2 having an amino acid sequence 100% identical to the amino acid sequence of DTS, and a CDR L3 having an amino acid sequence 100% identical to the amino acid sequence of SEQ ID NO: 312.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 314, a CDR H2 having an amino acid sequence 100% identical to the amino acid sequence of SEQ ID NO: 315, and a CDR H3 having an amino acid sequence 100% identical to the amino acid sequence of SEQ ID NO: 316; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 318, a CDR L2 having an amino acid sequence 100% identical to the amino acid sequence of DAS, and a CDR L3 having an amino acid sequence 100% identical to the amino acid sequence of SEQ ID NO: 319.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 321, a CDR H2 having an amino acid sequence 100% identical to the amino acid sequence of SEQ ID NO: 322, and a CDR H3 having an amino acid sequence 100% identical to the amino acid sequence of SEQ ID NO: 323; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 325, a CDR L2 having an amino acid sequence 100% identical to the amino acid sequence of STS, and a CDR L3 having an amino acid sequence 100% identical to the amino acid sequence of SEQ ID NO: 326.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 328, a CDR H2 having an amino acid sequence 100% identical to the amino acid sequence of SEQ ID NO: 329, and a CDR H3 having an amino acid sequence 100% identical to the amino acid sequence of SEQ ID NO: 330; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 332, a CDR L2 having an amino acid sequence 100% identical to the amino acid sequence of DAS, and a CDR L3 having an amino acid sequence 100% identical to the amino acid sequence of SEQ ID NO: 333.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 335, a CDR H2 having an amino acid sequence 100% identical to the amino acid sequence of SEQ ID NO: 336, and a CDR H3 having an amino acid sequence 100% identical to the amino acid sequence of SEQ ID NO: 337; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 339, a CDR L2 having an amino acid sequence 100% identical to the amino acid sequence of GAS, and a CDR L3 having an amino acid sequence 100% identical to the amino acid sequence of SEQ ID NO: 340.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 342, a CDR H2 having an amino acid sequence 100% identical to the amino acid sequence of SEQ ID NO: 343, and a CDR H3 having an amino acid sequence 100% identical to the amino acid sequence of SEQ ID NO: 344; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 346, a CDR L2 having an amino acid sequence 100% identical to the amino acid sequence of DAS, and a CDR L3 having an amino acid sequence 100% identical to the amino acid sequence of SEQ ID NO: 347.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 349, a CDR H2 having an amino acid sequence 100% identical to the amino acid sequence of SEQ ID NO: 350, and a CDR H3 having an amino acid sequence 100% identical to the amino acid sequence of SEQ ID NO: 351; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 353, a CDR L2 having an amino acid sequence 100% identical to the amino acid sequence of DTS, and a CDR L3 having an amino acid sequence 100% identical to the amino acid sequence of SEQ ID NO: 354.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 356, a CDR H2 having an amino acid sequence 100% identical to the amino acid sequence of SEQ ID NO: 357, and a CDR H3 having an amino acid sequence 100% identical to the amino acid sequence of SEQ ID NO: 358; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 360, a CDR L2 having an amino acid sequence 100% identical to the amino acid sequence of AAS, and a CDR L3 having an amino acid sequence 100% identical to the amino acid sequence of SEQ ID NO: 361.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 363, a CDR H2 having an amino acid sequence 100% identical to the amino acid sequence of SEQ ID NO: 364, and a CDR H3 having an amino acid sequence 100% identical to the amino acid sequence of SEQ ID NO: 365; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 367, a CDR L2 having an amino acid sequence 100% identical to the amino acid sequence of AAS, and a CDR L3 having an amino acid sequence 100% identical to the amino acid sequence of SEQ ID NO: 368.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 370, a CDR H2 having an amino acid sequence 100% identical to the amino acid sequence of SEQ ID NO: 371, and a CDR H3 having an amino acid sequence 100% identical to the amino acid sequence of SEQ ID NO: 372; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 374, a CDR L2 having an amino acid sequence 100% identical to the amino acid sequence of GTS, and a CDR L3 having an amino acid sequence 100% identical to the amino acid sequence of SEQ ID NO: 375.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 377, a CDR H2 having an amino acid sequence 100% identical to the amino acid sequence of SEQ ID NO: 378, and a CDR H3 having an amino acid sequence 100% identical to the amino acid sequence of SEQ ID NO: 379; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 381, a CDR L2 having an amino acid sequence 100% identical to the amino acid sequence of ETS, and a CDR L3 having an amino acid sequence 100% identical to the amino acid sequence of SEQ ID NO: 382.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 384, a CDR H2 having an amino acid sequence 100% identical to the amino acid sequence of SEQ ID NO: 385, and a CDR H3 having an amino acid sequence 100% identical to the amino acid sequence of SEQ ID NO: 386; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 388, a CDR L2 having an amino acid sequence 100% identical to the amino acid sequence of GAS, and a CDR L3 having an amino acid sequence 100% identical to the amino acid sequence of SEQ ID NO: 389.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 391, a CDR H2 having an amino acid sequence 100% identical to the amino acid sequence of SEQ ID NO: 392, and a CDR H3 having an amino acid sequence 100% identical to the amino acid sequence of SEQ ID NO: 393; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 395, a CDR L2 having an amino acid sequence 100% identical to the amino acid sequence of DAS, and a CDR L3 having an amino acid sequence 100% identical to the amino acid sequence of SEQ ID NO: 396.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 398, a CDR H2 having an amino acid sequence 100% identical to the amino acid sequence of SEQ ID NO: 399, and a CDR H3 having an amino acid sequence 100% identical to the amino acid sequence of SEQ ID NO: 400; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 402, a CDR L2 having an amino acid sequence 100% identical to the amino acid sequence of GAS, and a CDR L3 having an amino acid sequence 100% identical to the amino acid sequence of SEQ ID NO: 403.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 405, a CDR H2 having an amino acid sequence 100% identical to the amino acid sequence of SEQ ID NO: 406, and a CDR H3 having an amino acid sequence 100% identical to the amino acid sequence of SEQ ID NO: 407; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 409, a CDR L2 having an amino acid sequence 100% identical to the amino acid sequence of DAS, and a CDR L3 having an amino acid sequence 100% identical to the amino acid sequence of SEQ ID NO: 410.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 412, a CDR H2 having an amino acid sequence 100% identical to the amino acid sequence of SEQ ID NO: 413, and a CDR H3 having an amino acid sequence 100% identical to the amino acid sequence of SEQ ID NO: 414; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 416, a CDR L2 having an amino acid sequence 100% identical to the amino acid sequence of DAF, and a CDR L3 having an amino acid sequence 100% identical to the amino acid sequence of SEQ ID NO: 417.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 419, a CDR H2 having an amino acid sequence 100% identical to the amino acid sequence of SEQ ID NO: 420, and a CDR H3 having an amino acid sequence 100% identical to the amino acid sequence of SEQ ID NO: 421; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 423, a CDR L2 having an amino acid sequence 100% identical to the amino acid sequence of DAS, and a CDR L3 having an amino acid sequence 100% identical to the amino acid sequence of SEQ ID NO: 424.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 426, a CDR H2 having an amino acid sequence 100% identical to the amino acid sequence of SEQ ID NO: 427, and a CDR H3 having an amino acid sequence 100% identical to the amino acid sequence of SEQ ID NO: 428; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 430, a CDR L2 having an amino acid sequence 100% identical to the amino acid sequence of DAS, and a CDR L3 having an amino acid sequence 100% identical to the amino acid sequence of SEQ ID NO: 431.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 433, a CDR H2 having an amino acid sequence 100% identical to the amino acid sequence of SEQ ID NO: 434, and a CDR H3 having an amino acid sequence 100% identical to the amino acid sequence of SEQ ID NO: 435; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 437, a CDR L2 having an amino acid sequence 100% identical to the amino acid sequence of GAS, and a CDR L3 having an amino acid sequence 100% identical to the amino acid sequence of SEQ ID NO: 438.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 440, a CDR H2 having an amino acid sequence 100% identical to the amino acid sequence of SEQ ID NO: 441, and a CDR H3 having an amino acid sequence 100% identical to the amino acid sequence of SEQ ID NO: 442; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 444, a CDR L2 having an amino acid sequence 100% identical to the amino acid sequence of SEQ ID NO: AAS, and a CDR L3 having an amino acid sequence 100% identical to the amino acid sequence of SEQ ID NO: 445.

In some embodiments, the antibody comprises a VH having at least 80% sequence identity to any one of the VH amino acid sequences listed in Table 1 and comprising therein a complementarity determining region (CDR) H1 having an amino acid sequence 100% identical to the amino acid sequence of SEQ ID NO: 447, a CDR H2 having an amino acid sequence 100% identical to the amino acid sequence of SEQ ID NO: 448, and a CDR H3 having an amino acid sequence 100% identical to the amino acid sequence of SEQ ID NO: 449; and a VL having at least 80% sequence identity to any one of the VL amino acid sequences listed in Table 1 and comprising therein a complementarity determining region (CDR) L1 having an amino acid sequence 100% identical to the amino acid sequence of SEQ ID NO: 451, a CDR L2 having an amino acid sequence 100% identical to the amino acid sequence of DNF, and a CDR L3 having an amino acid sequence 100% identical to the amino acid sequence of SEQ ID NO: 452.

In some embodiments, an antibody disclosed herein comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 12 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 16. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 12 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 16. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 12 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 16. In some embodiments, the antibody comprises a variable heavy chain (VH) region having an amino acid sequence with at least 95% (e.g., at least 95%, 96%, 97%, 98%, 99%, or more) identity to SEQ ID NO: 12 and a variable light chain (VL) region having an amino acid sequence with at least 95% (e.g., at least 95%, 96%, 97%, 98%, 99%, or more) identity to SEQ ID NO: 16. In some embodiments, the antibody comprises a variable heavy chain (VH) region having an amino acid sequence 100% identical to the amino acid sequence of SEQ ID NO: 12 and a variable light chain (VL) region having an amino acid sequence 100% identical to the amino acid sequence of SEQ ID NO: 16. In some embodiments, the antibody comprises a VH comprising therein a complementarity determining region (CDR) H1 having an amino acid sequence 100% identical to the amino acid sequence of SEQ ID NO: 13, a CDR H2 having an amino acid sequence 100% identical to the amino acid sequence of SEQ ID NO: 14, and a CDR H3 having an amino acid sequence 100% identical to the amino acid sequence of SEQ ID NO: 15; and a VL comprising therein a complementarity determining region (CDR) L1 having an amino acid sequence 100% identical to the amino acid sequence of SEQ ID NO: 17, a CDR L2 having an amino acid sequence 100% identical to the amino acid sequence of KAS, and a CDR L3 having an amino acid sequence 100% identical to the amino acid sequence of SEQ ID NO: 18. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 453, a second heavy chain region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 473, and a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 463.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 19 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 23. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 19 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 23. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 19 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 23. In some embodiments, the antibody comprises a variable heavy chain (VH) region having an amino acid sequence with at least 95% (e.g., at least 95%, 96%, 97%, 98%, 99%, or more) identity to SEQ ID NO: 19 and a variable light chain (VL) region having an amino acid sequence with at least 95% (e.g., at least 95%, 96%, 97%, 98%, 99%, or more) identity to SEQ ID NO: 23. In some embodiments, the antibody comprises a variable heavy chain (VH) region having an amino acid sequence 100% identical to the amino acid sequence of SEQ ID NO: 19 and a variable light chain (VL) region having an amino acid sequence 100% identical to the amino acid sequence of SEQ ID NO: 23. In some embodiments, the antibody comprises a VH comprising therein a complementarity determining region (CDR) H1 having an amino acid sequence 100% identical to the amino acid sequence of SEQ ID NO: 20, a CDR H2 having an amino acid sequence 100% identical to the amino acid sequence of SEQ ID NO: 21, and a CDR H3 having an amino acid sequence 100% identical to the amino acid sequence of SEQ ID NO: 22; and a VL comprising therein a complementarity determining region (CDR) L1 having an amino acid sequence 100% identical to the amino acid sequence of SEQ ID NO: 24, a CDR L2 having an amino acid sequence 100% identical to the amino acid sequence of AAS, and a CDR L3 having an amino acid sequence 100% identical to the amino acid sequence of SEQ ID NO: 25. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 454, a second heavy chain region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 474, and a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 464.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 26 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 30. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 26 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 30. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 26 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 30. In some embodiments, the antibody comprises a variable heavy chain (VH) region having an amino acid sequence with at least 95% (e.g., at least 95%, 96%, 97%, 98%, 99%, or more) identity to SEQ ID NO: 26 and a variable light chain (VL) region having an amino acid sequence with at least 95% (e.g., at least 95%, 96%, 97%, 98%, 99%, or more) identity to SEQ ID NO: 30. In some embodiments, the antibody comprises a variable heavy chain (VH) region having an amino acid sequence 100% identical to the amino acid sequence of SEQ ID NO: 26 and a variable light chain (VL) region having an amino acid sequence 100% identical to the amino acid sequence of SEQ ID NO: 30. In some embodiments, the antibody comprises a VH comprising therein a complementarity determining region (CDR) H1 having an amino acid sequence 100% identical to the amino acid sequence of SEQ ID NO: 27, a CDR H2 having an amino acid sequence 100% identical to the amino acid sequence of SEQ ID NO: 28, and a CDR H3 having an amino acid sequence 100% identical to the amino acid sequence of SEQ ID NO: 29; and a VL comprising therein a complementarity determining region (CDR) L1 having an amino acid sequence 100% identical to the amino acid sequence of SEQ ID NO: 31, a CDR L2 having an amino acid sequence 100% identical to the amino acid sequence of ESS, and a CDR L3 having an amino acid sequence 100% identical to the amino acid sequence of SEQ ID NO: 32.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 33 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 37. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 33 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 37. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 33 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 37. In some embodiments, the antibody comprises a variable heavy chain (VH) region having an amino acid sequence with at least 95% (e.g., at least 95%, 96%, 97%, 98%, 99%, or more) identity to SEQ ID NO: 33 and a variable light chain (VL) region having an amino acid sequence with at least 95% (e.g., at least 95%, 96%, 97%, 98%, 99%, or more) identity to SEQ ID NO: 37. In some embodiments, the antibody comprises a variable heavy chain (VH) region having an amino acid sequence 100% identical to the amino acid sequence of SEQ ID NO: 33 and a variable light chain (VL) region having an amino acid sequence 100% identical to the amino acid sequence of SEQ ID NO: 37. In some embodiments, the antibody comprises a VH comprising therein a complementarity determining region (CDR) H1 having an amino acid sequence 100% identical to the amino acid sequence of SEQ ID NO: 34, a CDR H2 having an amino acid sequence 100% identical to the amino acid sequence of SEQ ID NO: 35, and a CDR H3 having an amino acid sequence 100% identical to the amino acid sequence of SEQ ID NO: 36; and a VL comprising therein a complementarity determining region (CDR) L1 having an amino acid sequence 100% identical to the amino acid sequence of SEQ ID NO: 38, a CDR L2 having an amino acid sequence 100% identical to the amino acid sequence of DAT, and a CDR L3 having an amino acid sequence 100% identical to the amino acid sequence of SEQ ID NO: 39. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 455, a second heavy chain region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 475, and a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 465.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 40 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 44. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 40 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 44. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 40 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 44. In some embodiments, the antibody comprises a variable heavy chain (VH) region having an amino acid sequence with at least 95% (e.g., at least 95%, 96%, 97%, 98%, 99%, or more) identity to SEQ ID NO: 40 and a variable light chain (VL) region having an amino acid sequence with at least 95% (e.g., at least 95%, 96%, 97%, 98%, 99%, or more) identity to SEQ ID NO: 44. In some embodiments, the antibody comprises a variable heavy chain (VH) region having an amino acid sequence 100% identical to the amino acid sequence of SEQ ID NO: 40 and a variable light chain (VL) region having an amino acid sequence 100% identical to the amino acid sequence of SEQ ID NO: 44. In some embodiments, the antibody comprises a VH comprising therein a complementarity determining region (CDR) H1 having an amino acid sequence 100% identical to the amino acid sequence of SEQ ID NO: 41, a CDR H2 having an amino acid sequence 100% identical to the amino acid sequence of SEQ ID NO: 42, and a CDR H3 having an amino acid sequence 100% identical to the amino acid sequence of SEQ ID NO: 43; and a VL comprising therein a complementarity determining region (CDR) L1 having an amino acid sequence 100% identical to the amino acid sequence of SEQ ID NO: 45, a CDR L2 having an amino acid sequence 100% identical to the amino acid sequence of GSS, and a CDR L3 having an amino acid sequence 100% identical to the amino acid sequence of SEQ ID NO: 46. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 456, a second heavy chain region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 476, and a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 466.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 47 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 51. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 47 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 51. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 47 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 51. In some embodiments, the antibody comprises a variable heavy chain (VH) region having an amino acid sequence with at least 95% (e.g., at least 95%, 96%, 97%, 98%, 99%, or more) identity to SEQ ID NO: 47 and a variable light chain (VL) region having an amino acid sequence with at least 95% (e.g., at least 95%, 96%, 97%, 98%, 99%, or more) identity to SEQ ID NO: 51. In some embodiments, the antibody comprises a variable heavy chain (VH) region having an amino acid sequence 100% identical to the amino acid sequence of SEQ ID NO: 47 and a variable light chain (VL) region having an amino acid sequence 100% identical to the amino acid sequence of SEQ ID NO: 51. In some embodiments, the antibody comprises a VH comprising therein a complementarity determining region (CDR) H1 having an amino acid sequence 100% identical to the amino acid sequence of SEQ ID NO: 48, a CDR H2 having an amino acid sequence 100% identical to the amino acid sequence of SEQ ID NO: 49, and a CDR H3 having an amino acid sequence 100% identical to the amino acid sequence of SEQ ID NO: 50; and a VL comprising therein a complementarity determining region (CDR) L1 having an amino acid sequence 100% identical to the amino acid sequence of SEQ ID NO: 52, a CDR L2 having an amino acid sequence 100% identical to the amino acid sequence of SEQ ID NO: DAT, and a CDR L3 having an amino acid sequence 100% identical to the amino acid sequence of SEQ ID NO: 53.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 54 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 58. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 54 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 58. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 54 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 58. In some embodiments, the antibody comprises a variable heavy chain (VH) region having an amino acid sequence with at least 95% (e.g., at least 95%, 96%, 97%, 98%, 99%, or more) identity to SEQ ID NO: 54 and a variable light chain (VL) region having an amino acid sequence with at least 95% (e.g., at least 95%, 96%, 97%, 98%, 99%, or more) identity to SEQ ID NO: 58. In some embodiments, the antibody comprises a variable heavy chain (VH) region having an amino acid sequence 100% identical to the amino acid sequence of SEQ ID NO: 54 and a variable light chain (VL) region having an amino acid sequence 100% identical to the amino acid sequence of SEQ ID NO: 58. In some embodiments, the antibody comprises a VH comprising therein a complementarity determining region (CDR) H1 having an amino acid sequence 100% identical to the amino acid sequence of SEQ ID NO: 55, a CDR H2 having an amino acid sequence 100% identical to the amino acid sequence of SEQ ID NO: 56, and a CDR H3 having an amino acid sequence 100% identical to the amino acid sequence of SEQ ID NO: 57; and a VL comprising therein a complementarity determining region (CDR) L1 having an amino acid sequence 100% identical to the amino acid sequence of SEQ ID NO: 59, a CDR L2 having an amino acid sequence 100% identical to the amino acid sequence of GAS, and a CDR L3 having an amino acid sequence 100% identical to the amino acid sequence of SEQ ID NO: 60. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 457, a second heavy chain region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 477, and a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 467.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 61 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 65. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 61 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 65. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 61 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 65. In some embodiments, the antibody comprises a variable heavy chain (VH) region having an amino acid sequence with at least 95% (e.g., at least 95%, 96%, 97%, 98%, 99%, or more) identity to SEQ ID NO: 61 and a variable light chain (VL) region having an amino acid sequence with at least 95% (e.g., at least 95%, 96%, 97%, 98%, 99%, or more) identity to SEQ ID NO: 65. In some embodiments, the antibody comprises a variable heavy chain (VH) region having an amino acid sequence 100% identical to the amino acid sequence of SEQ ID NO: 61 and a variable light chain (VL) region having an amino acid sequence 100% identical to the amino acid sequence of SEQ ID NO: 65. In some embodiments, the antibody comprises a VH comprising therein a complementarity determining region (CDR) H1 having an amino acid sequence 100% identical to the amino acid sequence of SEQ ID NO: 62, a CDR H2 having an amino acid sequence 100% identical to the amino acid sequence of SEQ ID NO: 63, and a CDR H3 having an amino acid sequence 100% identical to the amino acid sequence of SEQ ID NO: 64; and a VL comprising therein a complementarity determining region (CDR) L1 having an amino acid sequence 100% identical to the amino acid sequence of SEQ ID NO: 66, a CDR L2 having an amino acid sequence 100% identical to the amino acid sequence of DAS, and a CDR L3 having an amino acid sequence 100% identical to the amino acid sequence of SEQ ID NO: 67.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 68 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 72. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 68 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 72. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 68 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 72. In some embodiments, the antibody comprises a variable heavy chain (VH) region having an amino acid sequence with at least 95% (e.g., at least 95%, 96%, 97%, 98%, 99%, or more) identity to SEQ ID NO: 68 and a variable light chain (VL) region having an amino acid sequence with at least 95% (e.g., at least 95%, 96%, 97%, 98%, 99%, or more) identity to SEQ ID NO: 72. In some embodiments, the antibody comprises a variable heavy chain (VH) region having an amino acid sequence 100% identical to the amino acid sequence of SEQ ID NO: 68 and a variable light chain (VL) region having an amino acid sequence 100% identical to the amino acid sequence of SEQ ID NO: 72. In some embodiments, the antibody comprises a VH comprising therein a complementarity determining region (CDR) H1 having an amino acid sequence 100% identical to the amino acid sequence of SEQ ID NO: 69, a CDR H2 having an amino acid sequence 100% identical to the amino acid sequence of SEQ ID NO: 70, and a CDR H3 having an amino acid sequence 100% identical to the amino acid sequence of SEQ ID NO: 71; and a VL comprising therein a complementarity determining region (CDR) L1 having an amino acid sequence 100% identical to the amino acid sequence of SEQ ID NO: 73, a CDR L2 having an amino acid sequence 100% identical to the amino acid sequence of GAS, and a CDR L3 having an amino acid sequence 100% identical to the amino acid sequence of SEQ ID NO: 72.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 75 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 79. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 75 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 79. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 75 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 79. In some embodiments, the antibody comprises a variable heavy chain (VH) region having an amino acid sequence with at least 95% (e.g., at least 95%, 96%, 97%, 98%, 99%, or more) identity to SEQ ID NO: 75 and a variable light chain (VL) region having an amino acid sequence with at least 95% (e.g., at least 95%, 96%, 97%, 98%, 99%, or more) identity to SEQ ID NO: 79. In some embodiments, the antibody comprises a variable heavy chain (VH) region having an amino acid sequence 100% identical to the amino acid sequence of SEQ ID NO: 75 and a variable light chain (VL) region having an amino acid sequence 100% identical to the amino acid sequence of SEQ ID NO: 79. In some embodiments, the antibody comprises a VH comprising therein a complementarity determining region (CDR) H1 having an amino acid sequence 100% identical to the amino acid sequence of SEQ ID NO: 76, a CDR H2 having an amino acid sequence 100% identical to the amino acid sequence of SEQ ID NO: 77, and a CDR H3 having an amino acid sequence 100% identical to the amino acid sequence of SEQ ID NO: 78; and a VL comprising therein a complementarity determining region (CDR) L1 having an amino acid sequence 100% identical to the amino acid sequence of SEQ ID NO: 80, a CDR L2 having an amino acid sequence 100% identical to the amino acid sequence of HAS, and a CDR L3 having an amino acid sequence 100% identical to the amino acid sequence of SEQ ID NO: 81.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 82 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 86. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 82 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 86. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 82 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 86. In some embodiments, the antibody comprises a variable heavy chain (VH) region having an amino acid sequence with at least 95% (e.g., at least 95%, 96%, 97%, 98%, 99%, or more) identity to SEQ ID NO: 82 and a variable light chain (VL) region having an amino acid sequence with at least 95% (e.g., at least 95%, 96%, 97%, 98%, 99%, or more) identity to SEQ ID NO: 86. In some embodiments, the antibody comprises a variable heavy chain (VH) region having an amino acid sequence 100% identical to the amino acid sequence of SEQ ID NO: 82 and a variable light chain (VL) region having an amino acid sequence 100% identical to the amino acid sequence of SEQ ID NO: 86. In some embodiments, the antibody comprises a VH comprising therein a complementarity determining region (CDR) H1 having an amino acid sequence 100% identical to the amino acid sequence of SEQ ID NO: 83, a CDR H2 having an amino acid sequence 100% identical to the amino acid sequence of SEQ ID NO: 84, and a CDR H3 having an amino acid sequence 100% identical to the amino acid sequence of SEQ ID NO: 85; and a VL comprising therein a complementarity determining region (CDR) L1 having an amino acid sequence 100% identical to the amino acid sequence of SEQ ID NO: 87, a CDR L2 having an amino acid sequence 100% identical to the amino acid sequence of DAT, and a CDR L3 having an amino acid sequence 100% identical to the amino acid sequence of SEQ ID NO: 88.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 89 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 93. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 89 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 93. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 89 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 93. In some embodiments, the antibody comprises a variable heavy chain (VH) region having an amino acid sequence with at least 95% (e.g., at least 95%, 96%, 97%, 98%, 99%, or more) identity to SEQ ID NO: 89 and a variable light chain (VL) region having an amino acid sequence with at least 95% (e.g., at least 95%, 96%, 97%, 98%, 99%, or more) identity to SEQ ID NO: 93. In some embodiments, the antibody comprises a variable heavy chain (VH) region having an amino acid sequence 100% identical to the amino acid sequence of SEQ ID NO: 89 and a variable light chain (VL) region having an amino acid sequence 100% identical to the amino acid sequence of SEQ ID NO: 93. In some embodiments, the antibody comprises a VH comprising therein a complementarity determining region (CDR) H1 having an amino acid sequence 100% identical to the amino acid sequence of SEQ ID NO: 90, a CDR H2 having an amino acid sequence 100% identical to the amino acid sequence of SEQ ID NO: 91, and a CDR H3 having an amino acid sequence 100% identical to the amino acid sequence of SEQ ID NO: 92; and a VL comprising therein a complementarity determining region (CDR) L1 having an amino acid sequence 100% identical to the amino acid sequence of SEQ ID NO: 94, a CDR L2 having an amino acid sequence 100% identical to the amino acid sequence of GAS, and a CDR L3 having an amino acid sequence 100% identical to the amino acid sequence of SEQ ID NO: 95. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 458, a second heavy chain region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 478, and a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 468.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 96 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 100. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 96 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 100. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 96 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 100. In some embodiments, the antibody comprises a variable heavy chain (VH) region having an amino acid sequence with at least 95% (e.g., at least 95%, 96%, 97%, 98%, 99%, or more) identity to SEQ ID NO: 96 and a variable light chain (VL) region having an amino acid sequence with at least 95% (e.g., at least 95%, 96%, 97%, 98%, 99%, or more) identity to SEQ ID NO: 100. In some embodiments, the antibody comprises a variable heavy chain (VH) region having an amino acid sequence 100% identical to the amino acid sequence of SEQ ID NO: 96 and a variable light chain (VL) region having an amino acid sequence 100% identical to the amino acid sequence of SEQ ID NO: 100. In some embodiments, the antibody comprises a VH comprising therein a complementarity determining region (CDR) H1 having an amino acid sequence 100% identical to the amino acid sequence of SEQ ID NO: 97, a CDR H2 having an amino acid sequence 100% identical to the amino acid sequence of SEQ ID NO: 98, and a CDR H3 having an amino acid sequence 100% identical to the amino acid sequence of SEQ ID NO: 99; and a VL comprising therein a complementarity determining region (CDR) L1 having an amino acid sequence 100% identical to the amino acid sequence of SEQ ID NO: 101, a CDR L2 having an amino acid sequence 100% identical to the amino acid sequence of DAS, and a CDR L3 having an amino acid sequence 100% identical to the amino acid sequence of SEQ ID NO: 102.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 103 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 107. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 103 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 107. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 103 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 107. In some embodiments, the antibody comprises a variable heavy chain (VH) region having an amino acid sequence with at least 95% (e.g., at least 95%, 96%, 97%, 98%, 99%, or more) identity to SEQ ID NO: 103 and a variable light chain (VL) region having an amino acid sequence with at least 95% (e.g., at least 95%, 96%, 97%, 98%, 99%, or more) identity to SEQ ID NO: 107. In some embodiments, the antibody comprises a variable heavy chain (VH) region having an amino acid sequence 100% identical to the amino acid sequence of SEQ ID NO: 103 and a variable light chain (VL) region having an amino acid sequence 100% identical to the amino acid sequence of SEQ ID NO: 107. In some embodiments, the antibody comprises a VH comprising therein a complementarity determining region (CDR) H1 having an amino acid sequence 100% identical to the amino acid sequence of SEQ ID NO: 104, a CDR H2 having an amino acid sequence 100% identical to the amino acid sequence of SEQ ID NO: 105, and a CDR H3 having an amino acid sequence 100% identical to the amino acid sequence of SEQ ID NO: 106; and a VL comprising therein a complementarity determining region (CDR) L1 having an amino acid sequence 100% identical to the amino acid sequence of SEQ ID NO: 108, a CDR L2 having an amino acid sequence 100% identical to the amino acid sequence of DIS, and a CDR L3 having an amino acid sequence 100% identical to the amino acid sequence of SEQ ID NO: 109.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 110 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 114. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 110 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 114. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 110 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 114. In some embodiments, the antibody comprises a variable heavy chain (VH) region having an amino acid sequence with at least 95% (e.g., at least 95%, 96%, 97%, 98%, 99%, or more) identity to SEQ ID NO: 110 and a variable light chain (VL) region having an amino acid sequence with at least 95% (e.g., at least 95%, 96%, 97%, 98%, 99%, or more) identity to SEQ ID NO: 114. In some embodiments, the antibody comprises a variable heavy chain (VH) region having an amino acid sequence 100% identical to the amino acid sequence of SEQ ID NO: 110 and a variable light chain (VL) region having an amino acid sequence 100% identical to the amino acid sequence of SEQ ID NO: 114. In some embodiments, the antibody comprises a VH comprising therein a complementarity determining region (CDR) H1 having an amino acid sequence 100% identical to the amino acid sequence of SEQ ID NO: 111, a CDR H2 having an amino acid sequence 100% identical to the amino acid sequence of SEQ ID NO: 112, and a CDR H3 having an amino acid sequence 100% identical to the amino acid sequence of SEQ ID NO: 113; and a VL comprising therein a complementarity determining region (CDR) L1 having an amino acid sequence 100% identical to the amino acid sequence of SEQ ID NO: 115, a CDR L2 having an amino acid sequence 100% identical to the amino acid sequence of DAS, and a CDR L3 having an amino acid sequence 100% identical to the amino acid sequence of SEQ ID NO: 116. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 459, a second heavy chain region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 479, and a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 469.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 117 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 121. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 117 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 121. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 117 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 121. In some embodiments, the antibody comprises a variable heavy chain (VH) region having an amino acid sequence with at least 95% (e.g., at least 95%, 96%, 97%, 98%, 99%, or more) identity to SEQ ID NO: 117 and a variable light chain (VL) region having an amino acid sequence with at least 95% (e.g., at least 95%, 96%, 97%, 98%, 99%, or more) identity to SEQ ID NO: 121. In some embodiments, the antibody comprises a variable heavy chain (VH) region having an amino acid sequence 100% identical to the amino acid sequence of SEQ ID NO: 117 and a variable light chain (VL) region having an amino acid sequence 100% identical to the amino acid sequence of SEQ ID NO: 121. In some embodiments, the antibody comprises a VH comprising therein a complementarity determining region (CDR) H1 having an amino acid sequence 100% identical to the amino acid sequence of SEQ ID NO: 118, a CDR H2 having an amino acid sequence 100% identical to the amino acid sequence of SEQ ID NO: 119, and a CDR H3 having an amino acid sequence 100% identical to the amino acid sequence of SEQ ID NO: 120; and a VL comprising therein a complementarity determining region (CDR) L1 having an amino acid sequence 100% identical to the amino acid sequence of SEQ ID NO: 122, a CDR L2 having an amino acid sequence 100% identical to the amino acid sequence of DAS, and a CDR L3 having an amino acid sequence 100% identical to the amino acid sequence of SEQ ID NO: 123.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 124 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 128. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 124 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 128. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 124 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 128. In some embodiments, the antibody comprises a variable heavy chain (VH) region having an amino acid sequence with at least 95% (e.g., at least 95%, 96%, 97%, 98%, 99%, or more) identity to SEQ ID NO: 124 and a variable light chain (VL) region having an amino acid sequence with at least 95% (e.g., at least 95%, 96%, 97%, 98%, 99%, or more) identity to SEQ ID NO: 128. In some embodiments, the antibody comprises a variable heavy chain (VH) region having an amino acid sequence 100% identical to the amino acid sequence of SEQ ID NO: 124 and a variable light chain (VL) region having an amino acid sequence 100% identical to the amino acid sequence of SEQ ID NO: 128. In some embodiments, the antibody comprises a VH comprising therein a complementarity determining region (CDR) H1 having an amino acid sequence 100% identical to the amino acid sequence of SEQ ID NO: 125, a CDR H2 having an amino acid sequence 100% identical to the amino acid sequence of SEQ ID NO: 126, and a CDR H3 having an amino acid sequence 100% identical to the amino acid sequence of SEQ ID NO: 127; and a VL comprising therein a complementarity determining region (CDR) L1 having an amino acid sequence 100% identical to the amino acid sequence of SEQ ID NO: 129, a CDR L2 having an amino acid sequence 100% identical to the amino acid sequence of EAS, and a CDR L3 having an amino acid sequence 100% identical to the amino acid sequence of SEQ ID NO: 130.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 131 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 135. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 131 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 135. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 131 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 135. In some embodiments, the antibody comprises a variable heavy chain (VH) region having an amino acid sequence with at least 95% (e.g., at least 95%, 96%, 97%, 98%, 99%, or more) identity to SEQ ID NO: 131 and a variable light chain (VL) region having an amino acid sequence with at least 95% (e.g., at least 95%, 96%, 97%, 98%, 99%, or more) identity to SEQ ID NO: 135. In some embodiments, the antibody comprises a variable heavy chain (VH) region having an amino acid sequence 100% identical to the amino acid sequence of SEQ ID NO: 131 and a variable light chain (VL) region having an amino acid sequence 100% identical to the amino acid sequence of SEQ ID NO: 135. In some embodiments, the antibody comprises a VH comprising therein a complementarity determining region (CDR) H1 having an amino acid sequence 100% identical to the amino acid sequence of SEQ ID NO: 132, a CDR H2 having an amino acid sequence 100% identical to the amino acid sequence of SEQ ID NO: 133, and a CDR H3 having an amino acid sequence 100% identical to the amino acid sequence of SEQ ID NO: 134; and a VL comprising therein a complementarity determining region (CDR) L1 having an amino acid sequence 100% identical to the amino acid sequence of SEQ ID NO: 136, a CDR L2 having an amino acid sequence 100% identical to the amino acid sequence of DAS, and a CDR L3 having an amino acid sequence 100% identical to the amino acid sequence of SEQ ID NO: 137.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 138 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 142. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 138 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 142. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 138 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 142. In some embodiments, the antibody comprises a variable heavy chain (VH) region having an amino acid sequence with at least 95% (e.g., at least 95%, 96%, 97%, 98%, 99%, or more) identity to SEQ ID NO: 138 and a variable light chain (VL) region having an amino acid sequence with at least 95% (e.g., at least 95%, 96%, 97%, 98%, 99%, or more) identity to SEQ ID NO: 142. In some embodiments, the antibody comprises a variable heavy chain (VH) region having an amino acid sequence 100% identical to the amino acid sequence of SEQ ID NO: 138 and a variable light chain (VL) region having an amino acid sequence 100% identical to the amino acid sequence of SEQ ID NO: 142. In some embodiments, the antibody comprises a VH comprising therein a complementarity determining region (CDR) H1 having an amino acid sequence 100% identical to the amino acid sequence of SEQ ID NO: 139, a CDR H2 having an amino acid sequence 100% identical to the amino acid sequence of SEQ ID NO: 140, and a CDR H3 having an amino acid sequence 100% identical to the amino acid sequence of SEQ ID NO: 141; and a VL comprising therein a complementarity determining region (CDR) L1 having an amino acid sequence 100% identical to the amino acid sequence of SEQ ID NO: 143, a CDR L2 having an amino acid sequence 100% identical to the amino acid sequence of DAS, and a CDR L3 having an amino acid sequence 100% identical to the amino acid sequence of SEQ ID NO: 144.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 145 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 149. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 145 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 149. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 145 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 149. In some embodiments, the antibody comprises a variable heavy chain (VH) region having an amino acid sequence with at least 95% (e.g., at least 95%, 96%, 97%, 98%, 99%, or more) identity to SEQ ID NO: 145 and a variable light chain (VL) region having an amino acid sequence with at least 95% (e.g., at least 95%, 96%, 97%, 98%, 99%, or more) identity to SEQ ID NO: 149. In some embodiments, the antibody comprises a variable heavy chain (VH) region having an amino acid sequence 100% identical to the amino acid sequence of SEQ ID NO: 145 and a variable light chain (VL) region having an amino acid sequence 100% identical to the amino acid sequence of SEQ ID NO: 149. In some embodiments, the antibody comprises a VH comprising therein a complementarity determining region (CDR) H1 having an amino acid sequence 100% identical to the amino acid sequence of SEQ ID NO: 146, a CDR H2 having an amino acid sequence 100% identical to the amino acid sequence of SEQ ID NO: 147, and a CDR H3 having an amino acid sequence 100% identical to the amino acid sequence of SEQ ID NO: 148; and a VL comprising therein a complementarity determining region (CDR) L1 having an amino acid sequence 100% identical to the amino acid sequence of SEQ ID NO: 150, a CDR L2 having an amino acid sequence 100% identical to the amino acid sequence of DAS, and a CDR L3 having an amino acid sequence 100% identical to the amino acid sequence of SEQ ID NO: 151.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 152 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 156. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 152 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 156. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 152 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 156. In some embodiments, the antibody comprises a variable heavy chain (VH) region having an amino acid sequence with at least 95% (e.g., at least 95%, 96%, 97%, 98%, 99%, or more) identity to SEQ ID NO: 152 and a variable light chain (VL) region having an amino acid sequence with at least 95% (e.g., at least 95%, 96%, 97%, 98%, 99%, or more) identity to SEQ ID NO: 156. In some embodiments, the antibody comprises a variable heavy chain (VH) region having an amino acid sequence 100% identical to the amino acid sequence of SEQ ID NO: 152 and a variable light chain (VL) region having an amino acid sequence 100% identical to the amino acid sequence of SEQ ID NO: 156. In some embodiments, the antibody comprises a VH comprising therein a complementarity determining region (CDR) H1 having an amino acid sequence 100% identical to the amino acid sequence of SEQ ID NO: 153, a CDR H2 having an amino acid sequence 100% identical to the amino acid sequence of SEQ ID NO: 154, and a CDR H3 having an amino acid sequence 100% identical to the amino acid sequence of SEQ ID NO: 155; and a VL comprising therein a complementarity determining region (CDR) L1 having an amino acid sequence 100% identical to the amino acid sequence of SEQ ID NO: 157, a CDR L2 having an amino acid sequence 100% identical to the amino acid sequence of DAS, and a CDR L3 having an amino acid sequence 100% identical to the amino acid sequence of SEQ ID NO: 158.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 159 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 163. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 159 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 163. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 159 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 163. In some embodiments, the antibody comprises a variable heavy chain (VH) region having an amino acid sequence with at least 95% (e.g., at least 95%, 96%, 97%, 98%, 99%, or more) identity to SEQ ID NO: 159 and a variable light chain (VL) region having an amino acid sequence with at least 95% (e.g., at least 95%, 96%, 97%, 98%, 99%, or more) identity to SEQ ID NO: 163. In some embodiments, the antibody comprises a variable heavy chain (VH) region having an amino acid sequence 100% identical to the amino acid sequence of SEQ ID NO: 159 and a variable light chain (VL) region having an amino acid sequence 100% identical to the amino acid sequence of SEQ ID NO: 163. In some embodiments, the antibody comprises a VH comprising therein a complementarity determining region (CDR) H1 having an amino acid sequence 100% identical to the amino acid sequence of SEQ ID NO: 160, a CDR H2 having an amino acid sequence 100% identical to the amino acid sequence of SEQ ID NO: 161, and a CDR H3 having an amino acid sequence 100% identical to the amino acid sequence of SEQ ID NO: 162; and a VL comprising therein a complementarity determining region (CDR) L1 having an amino acid sequence 100% identical to the amino acid sequence of SEQ ID NO: 164, a CDR L2 having an amino acid sequence 100% identical to the amino acid sequence of DAS, and a CDR L3 having an amino acid sequence 100% identical to the amino acid sequence of SEQ ID NO: 165.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 166 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 170. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 166 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 170. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 166 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 170. In some embodiments, the antibody comprises a variable heavy chain (VH) region having an amino acid sequence with at least 95% (e.g., at least 95%, 96%, 97%, 98%, 99%, or more) identity to SEQ ID NO: 166 and a variable light chain (VL) region having an amino acid sequence with at least 95% (e.g., at least 95%, 96%, 97%, 98%, 99%, or more) identity to SEQ ID NO: 170. In some embodiments, the antibody comprises a variable heavy chain (VH) region having an amino acid sequence 100% identical to the amino acid sequence of SEQ ID NO: 166 and a variable light chain (VL) region having an amino acid sequence 100% identical to the amino acid sequence of SEQ ID NO: 170. In some embodiments, the antibody comprises a VH comprising therein a complementarity determining region (CDR) H1 having an amino acid sequence 100% identical to the amino acid sequence of SEQ ID NO: 167, a CDR H2 having an amino acid sequence 100% identical to the amino acid sequence of SEQ ID NO: 168, and a CDR H3 having an amino acid sequence 100% identical to the amino acid sequence of SEQ ID NO: 169; and a VL comprising therein a complementarity determining region (CDR) L1 having an amino acid sequence 100% identical to the amino acid sequence of SEQ ID NO: 171, a CDR L2 having an amino acid sequence 100% identical to the amino acid sequence of GAS, and a CDR L3 having an amino acid sequence 100% identical to the amino acid sequence of SEQ ID NO: 172.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 173 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 177. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 173 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 177. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 173 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 177. In some embodiments, the antibody comprises a variable heavy chain (VH) region having an amino acid sequence with at least 95% (e.g., at least 95%, 96%, 97%, 98%, 99%, or more) identity to SEQ ID NO: 173 and a variable light chain (VL) region having an amino acid sequence with at least 95% (e.g., at least 95%, 96%, 97%, 98%, 99%, or more) identity to SEQ ID NO: 177. In some embodiments, the antibody comprises a variable heavy chain (VH) region having an amino acid sequence 100% identical to the amino acid sequence of SEQ ID NO: 173 and a variable light chain (VL) region having an amino acid sequence 100% identical to the amino acid sequence of SEQ ID NO: 177. In some embodiments, the antibody comprises a VH comprising therein a complementarity determining region (CDR) H1 having an amino acid sequence 100% identical to the amino acid sequence of SEQ ID NO: 174, a CDR H2 having an amino acid sequence 100% identical to the amino acid sequence of SEQ ID NO: 175, and a CDR H3 having an amino acid sequence 100% identical to the amino acid sequence of SEQ ID NO: 176; and a VL comprising therein a complementarity determining region (CDR) L1 having an amino acid sequence 100% identical to the amino acid sequence of SEQ ID NO: 178, a CDR L2 having an amino acid sequence 100% identical to the amino acid sequence of GAS, and a CDR L3 having an amino acid sequence 100% identical to the amino acid sequence of SEQ ID NO: 179.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 180 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 184. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 180 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 184. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 180 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 184. In some embodiments, the antibody comprises a variable heavy chain (VH) region having an amino acid sequence with at least 95% (e.g., at least 95%, 96%, 97%, 98%, 99%, or more) identity to SEQ ID NO: 180 and a variable light chain (VL) region having an amino acid sequence with at least 95% (e.g., at least 95%, 96%, 97%, 98%, 99%, or more) identity to SEQ ID NO: 184. In some embodiments, the antibody comprises a variable heavy chain (VH) region having an amino acid sequence 100% identical to the amino acid sequence of SEQ ID NO: 180 and a variable light chain (VL) region having an amino acid sequence 100% identical to the amino acid sequence of SEQ ID NO: 184. In some embodiments, the antibody comprises a VH comprising therein a complementarity determining region (CDR) H1 having an amino acid sequence 100% identical to the amino acid sequence of SEQ ID NO: 181, a CDR H2 having an amino acid sequence 100% identical to the amino acid sequence of SEQ ID NO: 182, and a CDR H3 having an amino acid sequence 100% identical to the amino acid sequence of SEQ ID NO: 183; and a VL comprising therein a complementarity determining region (CDR) L1 having an amino acid sequence 100% identical to the amino acid sequence of SEQ ID NO: 185, a CDR L2 having an amino acid sequence 100% identical to the amino acid sequence of DVS, and a CDR L3 having an amino acid sequence 100% identical to the amino acid sequence of SEQ ID NO: 186.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 187 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 191. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 187 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 191. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 187 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 191. In some embodiments, the antibody comprises a variable heavy chain (VH) region having an amino acid sequence with at least 95% (e.g., at least 95%, 96%, 97%, 98%, 99%, or more) identity to SEQ ID NO: 187 and a variable light chain (VL) region having an amino acid sequence with at least 95% (e.g., at least 95%, 96%, 97%, 98%, 99%, or more) identity to SEQ ID NO: 191. In some embodiments, the antibody comprises a variable heavy chain (VH) region having an amino acid sequence 100% identical to the amino acid sequence of SEQ ID NO: 187 and a variable light chain (VL) region having an amino acid sequence 100% identical to the amino acid sequence of SEQ ID NO: 191. In some embodiments, the antibody comprises a VH comprising therein a complementarity determining region (CDR) H1 having an amino acid sequence 100% identical to the amino acid sequence of SEQ ID NO: 188, a CDR H2 having an amino acid sequence 100% identical to the amino acid sequence of SEQ ID NO: 189, and a CDR H3 having an amino acid sequence 100% identical to the amino acid sequence of SEQ ID NO: 190; and a VL comprising therein a complementarity determining region (CDR) L1 having an amino acid sequence 100% identical to the amino acid sequence of SEQ ID NO: 192, a CDR L2 having an amino acid sequence 100% identical to the amino acid sequence of DAT, and a CDR L3 having an amino acid sequence 100% identical to the amino acid sequence of SEQ ID NO: 193.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 194 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 198. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 194 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 198. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 194 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 198. In some embodiments, the antibody comprises a variable heavy chain (VH) region having an amino acid sequence with at least 95% (e.g., at least 95%, 96%, 97%, 98%, 99%, or more) identity to SEQ ID NO: 194 and a variable light chain (VL) region having an amino acid sequence with at least 95% (e.g., at least 95%, 96%, 97%, 98%, 99%, or more) identity to SEQ ID NO: 198. In some embodiments, the antibody comprises a variable heavy chain (VH) region having an amino acid sequence 100% identical to the amino acid sequence of SEQ ID NO: 194 and a variable light chain (VL) region having an amino acid sequence 100% identical to the amino acid sequence of SEQ ID NO: 198. In some embodiments, the antibody comprises a VH comprising therein a complementarity determining region (CDR) H1 having an amino acid sequence 100% identical to the amino acid sequence of SEQ ID NO: 195, a CDR H2 having an amino acid sequence 100% identical to the amino acid sequence of SEQ ID NO: 196, and a CDR H3 having an amino acid sequence 100% identical to the amino acid sequence of SEQ ID NO: 197; and a VL comprising therein a complementarity determining region (CDR) L1 having an amino acid sequence 100% identical to the amino acid sequence of SEQ ID NO: 199, a CDR L2 having an amino acid sequence 100% identical to the amino acid sequence of DAS, and a CDR L3 having an amino acid sequence 100% identical to the amino acid sequence of SEQ ID NO: 200.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 201 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 205. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 201 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 205. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 201 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 205. In some embodiments, the antibody comprises a variable heavy chain (VH) region having an amino acid sequence with at least 95% (e.g., at least 95%, 96%, 97%, 98%, 99%, or more) identity to SEQ ID NO: 201 and a variable light chain (VL) region having an amino acid sequence with at least 95% (e.g., at least 95%, 96%, 97%, 98%, 99%, or more) identity to SEQ ID NO: 205. In some embodiments, the antibody comprises a variable heavy chain (VH) region having an amino acid sequence 100% identical to the amino acid sequence of SEQ ID NO: 201 and a variable light chain (VL) region having an amino acid sequence 100% identical to the amino acid sequence of SEQ ID NO: 205. In some embodiments, the antibody comprises a VH comprising therein a complementarity determining region (CDR) H1 having an amino acid sequence 100% identical to the amino acid sequence of SEQ ID NO: 202, a CDR H2 having an amino acid sequence 100% identical to the amino acid sequence of SEQ ID NO: 203, and a CDR H3 having an amino acid sequence 100% identical to the amino acid sequence of SEQ ID NO: 204; and a VL comprising therein a complementarity determining region (CDR) L1 having an amino acid sequence 100% identical to the amino acid sequence of SEQ ID NO: 206, a CDR L2 having an amino acid sequence 100% identical to the amino acid sequence of GVD, and a CDR L3 having an amino acid sequence 100% identical to the amino acid sequence of SEQ ID NO: 207.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 208 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 212. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 208 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 212. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 208 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 212. In some embodiments, the antibody comprises a variable heavy chain (VH) region having an amino acid sequence with at least 95% (e.g., at least 95%, 96%, 97%, 98%, 99%, or more) identity to SEQ ID NO: 208 and a variable light chain (VL) region having an amino acid sequence with at least 95% (e.g., at least 95%, 96%, 97%, 98%, 99%, or more) identity to SEQ ID NO: 212. In some embodiments, the antibody comprises a variable heavy chain (VH) region having an amino acid sequence 100% identical to the amino acid sequence of SEQ ID NO: 208 and a variable light chain (VL) region having an amino acid sequence 100% identical to the amino acid sequence of SEQ ID NO: 212. In some embodiments, the antibody comprises a VH comprising therein a complementarity determining region (CDR) H1 having an amino acid sequence 100% identical to the amino acid sequence of SEQ ID NO: 209, a CDR H2 having an amino acid sequence 100% identical to the amino acid sequence of SEQ ID NO: 210, and a CDR H3 having an amino acid sequence 100% identical to the amino acid sequence of SEQ ID NO: 211; and a VL comprising therein a complementarity determining region (CDR) L1 having an amino acid sequence 100% identical to the amino acid sequence of SEQ ID NO: 213, a CDR L2 having an amino acid sequence 100% identical to the amino acid sequence of DAS, and a CDR L3 having an amino acid sequence 100% identical to the amino acid sequence of SEQ ID NO: 214.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 215 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 219. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 215 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 219. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 215 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 219. In some embodiments, the antibody comprises a variable heavy chain (VH) region having an amino acid sequence with at least 95% (e.g., at least 95%, 96%, 97%, 98%, 99%, or more) identity to SEQ ID NO: 215 and a variable light chain (VL) region having an amino acid sequence with at least 95% (e.g., at least 95%, 96%, 97%, 98%, 99%, or more) identity to SEQ ID NO: 219. In some embodiments, the antibody comprises a variable heavy chain (VH) region having an amino acid sequence 100% identical to the amino acid sequence of SEQ ID NO: 215 and a variable light chain (VL) region having an amino acid sequence 100% identical to the amino acid sequence of SEQ ID NO: 219. In some embodiments, the antibody comprises a VH comprising therein a complementarity determining region (CDR) H1 having an amino acid sequence 100% identical to the amino acid sequence of SEQ ID NO: 216, a CDR H2 having an amino acid sequence 100% identical to the amino acid sequence of SEQ ID NO: 217, and a CDR H3 having an amino acid sequence 100% identical to the amino acid sequence of SEQ ID NO: 218; and a VL comprising therein a complementarity determining region (CDR) L1 having an amino acid sequence 100% identical to the amino acid sequence of SEQ ID NO: 220, a CDR L2 having an amino acid sequence 100% identical to the amino acid sequence of GAT, and a CDR L3 having an amino acid sequence 100% identical to the amino acid sequence of SEQ ID NO: 221.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 222 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 226. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 222 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 226. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 222 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 226. In some embodiments, the antibody comprises a variable heavy chain (VH) region having an amino acid sequence with at least 95% (e.g., at least 95%, 96%, 97%, 98%, 99%, or more) identity to SEQ ID NO: 222 and a variable light chain (VL) region having an amino acid sequence with at least 95% (e.g., at least 95%, 96%, 97%, 98%, 99%, or more) identity to SEQ ID NO: 226. In some embodiments, the antibody comprises a variable heavy chain (VH) region having an amino acid sequence 100% identical to the amino acid sequence of SEQ ID NO: 222 and a variable light chain (VL) region having an amino acid sequence 100% identical to the amino acid sequence of SEQ ID NO: 226. In some embodiments, the antibody comprises a VH comprising therein a complementarity determining region (CDR) H1 having an amino acid sequence 100% identical to the amino acid sequence of SEQ ID NO: 223, a CDR H2 having an amino acid sequence 100% identical to the amino acid sequence of SEQ ID NO: 224, and a CDR H3 having an amino acid sequence 100% identical to the amino acid sequence of SEQ ID NO: 225; and a VL comprising therein a complementarity determining region (CDR) L1 having an amino acid sequence 100% identical to the amino acid sequence of SEQ ID NO: 227, a CDR L2 having an amino acid sequence 100% identical to the amino acid sequence of DAS, and a CDR L3 having an amino acid sequence 100% identical to the amino acid sequence of SEQ ID NO: 228.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 229 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 233. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 229 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 233. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 229 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 233. In some embodiments, the antibody comprises a variable heavy chain (VH) region having an amino acid sequence with at least 95% (e.g., at least 95%, 96%, 97%, 98%, 99%, or more) identity to SEQ ID NO: 229 and a variable light chain (VL) region having an amino acid sequence with at least 95% (e.g., at least 95%, 96%, 97%, 98%, 99%, or more) identity to SEQ ID NO: 233. In some embodiments, the antibody comprises a variable heavy chain (VH) region having an amino acid sequence 100% identical to the amino acid sequence of SEQ ID NO: 229 and a variable light chain (VL) region having an amino acid sequence 100% identical to the amino acid sequence of SEQ ID NO: 233. In some embodiments, the antibody comprises a VH comprising therein a complementarity determining region (CDR) H1 having an amino acid sequence 100% identical to the amino acid sequence of SEQ ID NO: 230, a CDR H2 having an amino acid sequence 100% identical to the amino acid sequence of SEQ ID NO: 231, and a CDR H3 having an amino acid sequence 100% identical to the amino acid sequence of SEQ ID NO: 232; and a VL comprising therein a complementarity determining region (CDR) L1 having an amino acid sequence 100% identical to the amino acid sequence of SEQ ID NO: 234, a CDR L2 having an amino acid sequence 100% identical to the amino acid sequence of DAS, and a CDR L3 having an amino acid sequence 100% identical to the amino acid sequence of SEQ ID NO: 235.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 236 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 240. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 236 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 240. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 236 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 240. In some embodiments, the antibody comprises a variable heavy chain (VH) region having an amino acid sequence with at least 95% (e.g., at least 95%, 96%, 97%, 98%, 99%, or more) identity to SEQ ID NO: 236 and a variable light chain (VL) region having an amino acid sequence with at least 95% (e.g., at least 95%, 96%, 97%, 98%, 99%, or more) identity to SEQ ID NO: 240. In some embodiments, the antibody comprises a variable heavy chain (VH) region having an amino acid sequence 100% identical to the amino acid sequence of SEQ ID NO: 236 and a variable light chain (VL) region having an amino acid sequence 100% identical to the amino acid sequence of SEQ ID NO: 240. In some embodiments, the antibody comprises a VH comprising therein a complementarity determining region (CDR) H1 having an amino acid sequence 100% identical to the amino acid sequence of SEQ ID NO: 237, a CDR H2 having an amino acid sequence 100% identical to the amino acid sequence of SEQ ID NO: 238, and a CDR H3 having an amino acid sequence 100% identical to the amino acid sequence of SEQ ID NO: 239; and a VL comprising therein a complementarity determining region (CDR) L1 having an amino acid sequence 100% identical to the amino acid sequence of SEQ ID NO: 241, a CDR L2 having an amino acid sequence 100% identical to the amino acid sequence of GAS, and a CDR L3 having an amino acid sequence 100% identical to the amino acid sequence of SEQ ID NO: 242.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 243 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 247. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 243 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 247. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 243 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 247. In some embodiments, the antibody comprises a variable heavy chain (VH) region having an amino acid sequence with at least 95% (e.g., at least 95%, 96%, 97%, 98%, 99%, or more) identity to SEQ ID NO: 243 and a variable light chain (VL) region having an amino acid sequence with at least 95% (e.g., at least 95%, 96%, 97%, 98%, 99%, or more) identity to SEQ ID NO: 247. In some embodiments, the antibody comprises a variable heavy chain (VH) region having an amino acid sequence 100% identical to the amino acid sequence of SEQ ID NO: 243 and a variable light chain (VL) region having an amino acid sequence 100% identical to the amino acid sequence of SEQ ID NO: 247. In some embodiments, the antibody comprises a VH comprising therein a complementarity determining region (CDR) H1 having an amino acid sequence 100% identical to the amino acid sequence of SEQ ID NO: 244, a CDR H2 having an amino acid sequence 100% identical to the amino acid sequence of SEQ ID NO: 245, and a CDR H3 having an amino acid sequence 100% identical to the amino acid sequence of SEQ ID NO: 246; and a VL comprising therein a complementarity determining region (CDR) L1 having an amino acid sequence 100% identical to the amino acid sequence of SEQ ID NO: 248, a CDR L2 having an amino acid sequence 100% identical to the amino acid sequence of GAS, and a CDR L3 having an amino acid sequence 100% identical to the amino acid sequence of SEQ ID NO: 249. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 460, a second heavy chain region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 480, and a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 470.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 250 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 254. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 250 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 254. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 250 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 254. In some embodiments, the antibody comprises a variable heavy chain (VH) region having an amino acid sequence with at least 95% (e.g., at least 95%, 96%, 97%, 98%, 99%, or more) identity to SEQ ID NO: 250 and a variable light chain (VL) region having an amino acid sequence with at least 95% (e.g., at least 95%, 96%, 97%, 98%, 99%, or more) identity to SEQ ID NO: 254. In some embodiments, the antibody comprises a variable heavy chain (VH) region having an amino acid sequence 100% identical to the amino acid sequence of SEQ ID NO: 250 and a variable light chain (VL) region having an amino acid sequence 100% identical to the amino acid sequence of SEQ ID NO: 254. In some embodiments, the antibody comprises a VH comprising therein a complementarity determining region (CDR) H1 having an amino acid sequence 100% identical to the amino acid sequence of SEQ ID NO: 251, a CDR H2 having an amino acid sequence 100% identical to the amino acid sequence of SEQ ID NO: 252, and a CDR H3 having an amino acid sequence 100% identical to the amino acid sequence of SEQ ID NO: 253; and a VL comprising therein a complementarity determining region (CDR) L1 having an amino acid sequence 100% identical to the amino acid sequence of SEQ ID NO: 255, a CDR L2 having an amino acid sequence 100% identical to the amino acid sequence of GAT, and a CDR L3 having an amino acid sequence 100% identical to the amino acid sequence of SEQ ID NO: 256.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 257 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 261. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 257 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 261. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 257 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 261. In some embodiments, the antibody comprises a variable heavy chain (VH) region having an amino acid sequence with at least 95% (e.g., at least 95%, 96%, 97%, 98%, 99%, or more) identity to SEQ ID NO: 257 and a variable light chain (VL) region having an amino acid sequence with at least 95% (e.g., at least 95%, 96%, 97%, 98%, 99%, or more) identity to SEQ ID NO: 261. In some embodiments, the antibody comprises a variable heavy chain (VH) region having an amino acid sequence 100% identical to the amino acid sequence of SEQ ID NO: 257 and a variable light chain (VL) region having an amino acid sequence 100% identical to the amino acid sequence of SEQ ID NO: 261. In some embodiments, the antibody comprises a VH comprising therein a complementarity determining region (CDR) H1 having an amino acid sequence 100% identical to the amino acid sequence of SEQ ID NO: 258, a CDR H2 having an amino acid sequence 100% identical to the amino acid sequence of SEQ ID NO: 259, and a CDR H3 having an amino acid sequence 100% identical to the amino acid sequence of SEQ ID NO: 260; and a VL comprising therein a complementarity determining region (CDR) L1 having an amino acid sequence 100% identical to the amino acid sequence of SEQ ID NO: 262, a CDR L2 having an amino acid sequence 100% identical to the amino acid sequence of GAS, and a CDR L3 having an amino acid sequence 100% identical to the amino acid sequence of SEQ ID NO: 263.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 264 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 268. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 264 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 268. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 264 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 268. In some embodiments, the antibody comprises a variable heavy chain (VH) region having an amino acid sequence with at least 95% (e.g., at least 95%, 96%, 97%, 98%, 99%, or more) identity to SEQ ID NO: 264 and a variable light chain (VL) region having an amino acid sequence with at least 95% (e.g., at least 95%, 96%, 97%, 98%, 99%, or more) identity to SEQ ID NO: 268. In some embodiments, the antibody comprises a variable heavy chain (VH) region having an amino acid sequence 100% identical to the amino acid sequence of SEQ ID NO: 264 and a variable light chain (VL) region having an amino acid sequence 100% identical to the amino acid sequence of SEQ ID NO: 268. In some embodiments, the antibody comprises a VH comprising therein a complementarity determining region (CDR) H1 having an amino acid sequence 100% identical to the amino acid sequence of SEQ ID NO: 265, a CDR H2 having an amino acid sequence 100% identical to the amino acid sequence of SEQ ID NO: 266, and a CDR H3 having an amino acid sequence 100% identical to the amino acid sequence of SEQ ID NO: 267; and a VL comprising therein a complementarity determining region (CDR) L1 having an amino acid sequence 100% identical to the amino acid sequence of SEQ ID NO: 269, a CDR L2 having an amino acid sequence 100% identical to the amino acid sequence of DAV, and a CDR L3 having an amino acid sequence 100% identical to the amino acid sequence of SEQ ID NO: 270.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 271 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 275. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 271 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 275. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 271 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 275. In some embodiments, the antibody comprises a variable heavy chain (VH) region having an amino acid sequence with at least 95% (e.g., at least 95%, 96%, 97%, 98%, 99%, or more) identity to SEQ ID NO: 271 and a variable light chain (VL) region having an amino acid sequence with at least 95% (e.g., at least 95%, 96%, 97%, 98%, 99%, or more) identity to SEQ ID NO: 275. In some embodiments, the antibody comprises a variable heavy chain (VH) region having an amino acid sequence 100% identical to the amino acid sequence of SEQ ID NO: 271 and a variable light chain (VL) region having an amino acid sequence 100% identical to the amino acid sequence of SEQ ID NO: 275. In some embodiments, the antibody comprises a VH comprising therein a complementarity determining region (CDR) H1 having an amino acid sequence 100% identical to the amino acid sequence of SEQ ID NO: 272, a CDR H2 having an amino acid sequence 100% identical to the amino acid sequence of SEQ ID NO: 273, and a CDR H3 having an amino acid sequence 100% identical to the amino acid sequence of SEQ ID NO: 274; and a VL comprising therein a complementarity determining region (CDR) L1 having an amino acid sequence 100% identical to the amino acid sequence of SEQ ID NO: 276, a CDR L2 having an amino acid sequence 100% identical to the amino acid sequence of DAS, and a CDR L3 having an amino acid sequence 100% identical to the amino acid sequence of SEQ ID NO: 277.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 278 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 282. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 278 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 282. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 278 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 282. In some embodiments, the antibody comprises a variable heavy chain (VH) region having an amino acid sequence with at least 95% (e.g., at least 95%, 96%, 97%, 98%, 99%, or more) identity to SEQ ID NO: 278 and a variable light chain (VL) region having an amino acid sequence with at least 95% (e.g., at least 95%, 96%, 97%, 98%, 99%, or more) identity to SEQ ID NO: 282. In some embodiments, the antibody comprises a variable heavy chain (VH) region having an amino acid sequence 100% identical to the amino acid sequence of SEQ ID NO: 278 and a variable light chain (VL) region having an amino acid sequence 100% identical to the amino acid sequence of SEQ ID NO: 282. In some embodiments, the antibody comprises a VH comprising therein a complementarity determining region (CDR) H1 having an amino acid sequence 100% identical to the amino acid sequence of SEQ ID NO: 279, a CDR H2 having an amino acid sequence 100% identical to the amino acid sequence of SEQ ID NO: 280, and a CDR H3 having an amino acid sequence 100% identical to the amino acid sequence of SEQ ID NO: 281; and a VL comprising therein a complementarity determining region (CDR) L1 having an amino acid sequence 100% identical to the amino acid sequence of SEQ ID NO: 283, a CDR L2 having an amino acid sequence 100% identical to the amino acid sequence of DAS, and a CDR L3 having an amino acid sequence 100% identical to the amino acid sequence of SEQ ID NO: 284.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 285 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 289. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 285 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 289. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 285 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 289. In some embodiments, the antibody comprises a variable heavy chain (VH) region having an amino acid sequence with at least 95% (e.g., at least 95%, 96%, 97%, 98%, 99%, or more) identity to SEQ ID NO: 285 and a variable light chain (VL) region having an amino acid sequence with at least 95% (e.g., at least 95%, 96%, 97%, 98%, 99%, or more) identity to SEQ ID NO: 289. In some embodiments, the antibody comprises a variable heavy chain (VH) region having an amino acid sequence 100% identical to the amino acid sequence of SEQ ID NO: 285 and a variable light chain (VL) region having an amino acid sequence 100% identical to the amino acid sequence of SEQ ID NO: 289. In some embodiments, the antibody comprises a VH comprising therein a complementarity determining region (CDR) H1 having an amino acid sequence 100% identical to the amino acid sequence of SEQ ID NO: 286, a CDR H2 having an amino acid sequence 100% identical to the amino acid sequence of SEQ ID NO: 287, and a CDR H3 having an amino acid sequence 100% identical to the amino acid sequence of SEQ ID NO: 288; and a VL comprising therein a complementarity determining region (CDR) L1 having an amino acid sequence 100% identical to the amino acid sequence of SEQ ID NO: 290, a CDR L2 having an amino acid sequence 100% identical to the amino acid sequence of GAS, and a CDR L3 having an amino acid sequence 100% identical to the amino acid sequence of SEQ ID NO: 291.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 292 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 296. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 292 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 296. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 292 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 296. In some embodiments, the antibody comprises a variable heavy chain (VH) region having an amino acid sequence with at least 95% (e.g., at least 95%, 96%, 97%, 98%, 99%, or more) identity to SEQ ID NO: 292 and a variable light chain (VL) region having an amino acid sequence with at least 95% (e.g., at least 95%, 96%, 97%, 98%, 99%, or more) identity to SEQ ID NO: 296. In some embodiments, the antibody comprises a variable heavy chain (VH) region having an amino acid sequence 100% identical to the amino acid sequence of SEQ ID NO: 292 and a variable light chain (VL) region having an amino acid sequence 100% identical to the amino acid sequence of SEQ ID NO: 296. In some embodiments, the antibody comprises a VH comprising therein a complementarity determining region (CDR) H1 having an amino acid sequence 100% identical to the amino acid sequence of SEQ ID NO: 293, a CDR H2 having an amino acid sequence 100% identical to the amino acid sequence of SEQ ID NO: 294, and a CDR H3 having an amino acid sequence 100% identical to the amino acid sequence of SEQ ID NO: 295; and a VL comprising therein a complementarity determining region (CDR) L1 having an amino acid sequence 100% identical to the amino acid sequence of SEQ ID NO: 297, a CDR L2 having an amino acid sequence 100% identical to the amino acid sequence of KAS, and a CDR L3 having an amino acid sequence 100% identical to the amino acid sequence of SEQ ID NO: 298.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 299 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 303. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 299 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 303. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 299 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 303. In some embodiments, the antibody comprises a variable heavy chain (VH) region having an amino acid sequence with at least 95% (e.g., at least 95%, 96%, 97%, 98%, 99%, or more) identity to SEQ ID NO: 299 and a variable light chain (VL) region having an amino acid sequence with at least 95% (e.g., at least 95%, 96%, 97%, 98%, 99%, or more) identity to SEQ ID NO: 303. In some embodiments, the antibody comprises a variable heavy chain (VH) region having an amino acid sequence 100% identical to the amino acid sequence of SEQ ID NO: 299 and a variable light chain (VL) region having an amino acid sequence 100% identical to the amino acid sequence of SEQ ID NO: 303. In some embodiments, the antibody comprises a VH comprising therein a complementarity determining region (CDR) H1 having an amino acid sequence 100% identical to the amino acid sequence of SEQ ID NO: 300, a CDR H2 having an amino acid sequence 100% identical to the amino acid sequence of SEQ ID NO: 301, and a CDR H3 having an amino acid sequence 100% identical to the amino acid sequence of SEQ ID NO: 302; and a VL comprising therein a complementarity determining region (CDR) L1 having an amino acid sequence 100% identical to the amino acid sequence of SEQ ID NO: 304, a CDR L2 having an amino acid sequence 100% identical to the amino acid sequence of AAS, and a CDR L3 having an amino acid sequence 100% identical to the amino acid sequence of SEQ ID NO: 305.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 306 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 310. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 306 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 310. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 306 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 310. In some embodiments, the antibody comprises a variable heavy chain (VH) region having an amino acid sequence with at least 95% (e.g., at least 95%, 96%, 97%, 98%, 99%, or more) identity to SEQ ID NO: 306 and a variable light chain (VL) region having an amino acid sequence with at least 95% (e.g., at least 95%, 96%, 97%, 98%, 99%, or more) identity to SEQ ID NO: 310. In some embodiments, the antibody comprises a variable heavy chain (VH) region having an amino acid sequence 100% identical to the amino acid sequence of SEQ ID NO: 306 and a variable light chain (VL) region having an amino acid sequence 100% identical to the amino acid sequence of SEQ ID NO: 310. In some embodiments, the antibody comprises a VH comprising therein a complementarity determining region (CDR) H1 having an amino acid sequence 100% identical to the amino acid sequence of SEQ ID NO: 307, a CDR H2 having an amino acid sequence 100% identical to the amino acid sequence of SEQ ID NO: 308, and a CDR H3 having an amino acid sequence 100% identical to the amino acid sequence of SEQ ID NO: 309; and a VL comprising therein a complementarity determining region (CDR) L1 having an amino acid sequence 100% identical to the amino acid sequence of SEQ ID NO: 311, a CDR L2 having an amino acid sequence 100% identical to the amino acid sequence of DTS, and a CDR L3 having an amino acid sequence 100% identical to the amino acid sequence of SEQ ID NO: 312. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 461, a second heavy chain region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 481, and a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 471.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 313 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 317. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 313 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 317. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 313 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 317. In some embodiments, the antibody comprises a variable heavy chain (VH) region having an amino acid sequence with at least 95% (e.g., at least 95%, 96%, 97%, 98%, 99%, or more) identity to SEQ ID NO: 313 and a variable light chain (VL) region having an amino acid sequence with at least 95% (e.g., at least 95%, 96%, 97%, 98%, 99%, or more) identity to SEQ ID NO: 317. In some embodiments, the antibody comprises a variable heavy chain (VH) region having an amino acid sequence 100% identical to the amino acid sequence of SEQ ID NO: 313 and a variable light chain (VL) region having an amino acid sequence 100% identical to the amino acid sequence of SEQ ID NO: 317. In some embodiments, the antibody comprises a VH comprising therein a complementarity determining region (CDR) H1 having an amino acid sequence 100% identical to the amino acid sequence of SEQ ID NO: 314, a CDR H2 having an amino acid sequence 100% identical to the amino acid sequence of SEQ ID NO: 315, and a CDR H3 having an amino acid sequence 100% identical to the amino acid sequence of SEQ ID NO: 316; and a VL comprising therein a complementarity determining region (CDR) L1 having an amino acid sequence 100% identical to the amino acid sequence of SEQ ID NO: 318, a CDR L2 having an amino acid sequence 100% identical to the amino acid sequence of DAS, and a CDR L3 having an amino acid sequence 100% identical to the amino acid sequence of SEQ ID NO: 319.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 320 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 324. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 320 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 324. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 320 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 324. In some embodiments, the antibody comprises a variable heavy chain (VH) region having an amino acid sequence with at least 95% (e.g., at least 95%, 96%, 97%, 98%, 99%, or more) identity to SEQ ID NO: 320 and a variable light chain (VL) region having an amino acid sequence with at least 95% (e.g., at least 95%, 96%, 97%, 98%, 99%, or more) identity to SEQ ID NO: 324. In some embodiments, the antibody comprises a variable heavy chain (VH) region having an amino acid sequence 100% identical to the amino acid sequence of SEQ ID NO: 320 and a variable light chain (VL) region having an amino acid sequence 100% identical to the amino acid sequence of SEQ ID NO: 324. In some embodiments, the antibody comprises a VH comprising therein a complementarity determining region (CDR) H1 having an amino acid sequence 100% identical to the amino acid sequence of SEQ ID NO: 321, a CDR H2 having an amino acid sequence 100% identical to the amino acid sequence of SEQ ID NO: 322, and a CDR H3 having an amino acid sequence 100% identical to the amino acid sequence of SEQ ID NO: 323; and a VL comprising therein a complementarity determining region (CDR) L1 having an amino acid sequence 100% identical to the amino acid sequence of SEQ ID NO: 325, a CDR L2 having an amino acid sequence 100% identical to the amino acid sequence of STS, and a CDR L3 having an amino acid sequence 100% identical to the amino acid sequence of SEQ ID NO: 326.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 327 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 331. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 327 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 331. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 327 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 331. In some embodiments, the antibody comprises a variable heavy chain (VH) region having an amino acid sequence with at least 95% (e.g., at least 95%, 96%, 97%, 98%, 99%, or more) identity to SEQ ID NO: 327 and a variable light chain (VL) region having an amino acid sequence with at least 95% (e.g., at least 95%, 96%, 97%, 98%, 99%, or more) identity to SEQ ID NO: 331. In some embodiments, the antibody comprises a variable heavy chain (VH) region having an amino acid sequence 100% identical to the amino acid sequence of SEQ ID NO: 327 and a variable light chain (VL) region having an amino acid sequence 100% identical to the amino acid sequence of SEQ ID NO: 331. In some embodiments, the antibody comprises a VH comprising therein a complementarity determining region (CDR) H1 having an amino acid sequence 100% identical to the amino acid sequence of SEQ ID NO: 328, a CDR H2 having an amino acid sequence 100% identical to the amino acid sequence of SEQ ID NO: 329, and a CDR H3 having an amino acid sequence 100% identical to the amino acid sequence of SEQ ID NO: 330; and a VL comprising therein a complementarity determining region (CDR) L1 having an amino acid sequence 100% identical to the amino acid sequence of SEQ ID NO: 332, a CDR L2 having an amino acid sequence 100% identical to the amino acid sequence of DAS, and a CDR L3 having an amino acid sequence 100% identical to the amino acid sequence of SEQ ID NO: 333.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 334 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 338. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 334 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 338. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 334 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 338. In some embodiments, the antibody comprises a variable heavy chain (VH) region having an amino acid sequence with at least 95% (e.g., at least 95%, 96%, 97%, 98%, 99%, or more) identity to SEQ ID NO: 334 and a variable light chain (VL) region having an amino acid sequence with at least 95% (e.g., at least 95%, 96%, 97%, 98%, 99%, or more) identity to SEQ ID NO: 338. In some embodiments, the antibody comprises a variable heavy chain (VH) region having an amino acid sequence 100% identical to the amino acid sequence of SEQ ID NO: 334 and a variable light chain (VL) region having an amino acid sequence 100% identical to the amino acid sequence of SEQ ID NO: 338. In some embodiments, the antibody comprises a VH comprising therein a complementarity determining region (CDR) H1 having an amino acid sequence 100% identical to the amino acid sequence of SEQ ID NO: 335, a CDR H2 having an amino acid sequence 100% identical to the amino acid sequence of SEQ ID NO: 336, and a CDR H3 having an amino acid sequence 100% identical to the amino acid sequence of SEQ ID NO: 337; and a VL comprising therein a complementarity determining region (CDR) L1 having an amino acid sequence 100% identical to the amino acid sequence of SEQ ID NO: 339, a CDR L2 having an amino acid sequence 100% identical to the amino acid sequence of GAS, and a CDR L3 having an amino acid sequence 100% identical to the amino acid sequence of SEQ ID NO: 340.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 341 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 345. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 341 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 345. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 341 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 345. In some embodiments, the antibody comprises a variable heavy chain (VH) region having an amino acid sequence with at least 95% (e.g., at least 95%, 96%, 97%, 98%, 99%, or more) identity to SEQ ID NO: 341 and a variable light chain (VL) region having an amino acid sequence with at least 95% (e.g., at least 95%, 96%, 97%, 98%, 99%, or more) identity to SEQ ID NO: 345. In some embodiments, the antibody comprises a variable heavy chain (VH) region having an amino acid sequence 100% identical to the amino acid sequence of SEQ ID NO: 341 and a variable light chain (VL) region having an amino acid sequence 100% identical to the amino acid sequence of SEQ ID NO: 345. In some embodiments, the antibody comprises a VH comprising therein a complementarity determining region (CDR) H1 having an amino acid sequence 100% identical to the amino acid sequence of SEQ ID NO: 342, a CDR H2 having an amino acid sequence 100% identical to the amino acid sequence of SEQ ID NO: 343, and a CDR H3 having an amino acid sequence 100% identical to the amino acid sequence of SEQ ID NO: 344; and a VL comprising therein a complementarity determining region (CDR) L1 having an amino acid sequence 100% identical to the amino acid sequence of SEQ ID NO: 346, a CDR L2 having an amino acid sequence 100% identical to the amino acid sequence of DAS, and a CDR L3 having an amino acid sequence 100% identical to the amino acid sequence of SEQ ID NO: 347.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 348 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 352. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 348 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 352. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 348 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 352. In some embodiments, the antibody comprises a variable heavy chain (VH) region having an amino acid sequence with at least 95% (e.g., at least 95%, 96%, 97%, 98%, 99%, or more) identity to SEQ ID NO: 348 and a variable light chain (VL) region having an amino acid sequence with at least 95% (e.g., at least 95%, 96%, 97%, 98%, 99%, or more) identity to SEQ ID NO: 352. In some embodiments, the antibody comprises a variable heavy chain (VH) region having an amino acid sequence 100% identical to the amino acid sequence of SEQ ID NO: 348 and a variable light chain (VL) region having an amino acid sequence 100% identical to the amino acid sequence of SEQ ID NO: 352. In some embodiments, the antibody comprises a VH comprising therein a complementarity determining region (CDR) H1 having an amino acid sequence 100% identical to the amino acid sequence of SEQ ID NO: 349, a CDR H2 having an amino acid sequence 100% identical to the amino acid sequence of SEQ ID NO: 350, and a CDR H3 having an amino acid sequence 100% identical to the amino acid sequence of SEQ ID NO: 351; and a VL comprising therein a complementarity determining region (CDR) L1 having an amino acid sequence 100% identical to the amino acid sequence of SEQ ID NO: 353, a CDR L2 having an amino acid sequence 100% identical to the amino acid sequence of DTS, and a CDR L3 having an amino acid sequence 100% identical to the amino acid sequence of SEQ ID NO: 354.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 355 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 359. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 355 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 359. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 355 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 359. In some embodiments, the antibody comprises a variable heavy chain (VH) region having an amino acid sequence with at least 95% (e.g., at least 95%, 96%, 97%, 98%, 99%, or more) identity to SEQ ID NO: 355 and a variable light chain (VL) region having an amino acid sequence with at least 95% (e.g., at least 95%, 96%, 97%, 98%, 99%, or more) identity to SEQ ID NO: 359. In some embodiments, the antibody comprises a variable heavy chain (VH) region having an amino acid sequence 100% identical to the amino acid sequence of SEQ ID NO: 355 and a variable light chain (VL) region having an amino acid sequence 100% identical to the amino acid sequence of SEQ ID NO: 359. In some embodiments, the antibody comprises a VH comprising therein a complementarity determining region (CDR) H1 having an amino acid sequence 100% identical to the amino acid sequence of SEQ ID NO: 356, a CDR H2 having an amino acid sequence 100% identical to the amino acid sequence of SEQ ID NO: 357, and a CDR H3 having an amino acid sequence 100% identical to the amino acid sequence of SEQ ID NO: 358; and a VL comprising therein a complementarity determining region (CDR) L1 having an amino acid sequence 100% identical to the amino acid sequence of SEQ ID NO: 360, a CDR L2 having an amino acid sequence 100% identical to the amino acid sequence of AAS, and a CDR L3 having an amino acid sequence 100% identical to the amino acid sequence of SEQ ID NO: 361.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 362 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 366. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 362 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 366. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 362 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 366. In some embodiments, the antibody comprises a variable heavy chain (VH) region having an amino acid sequence with at least 95% (e.g., at least 95%, 96%, 97%, 98%, 99%, or more) identity to SEQ ID NO: 362 and a variable light chain (VL) region having an amino acid sequence with at least 95% (e.g., at least 95%, 96%, 97%, 98%, 99%, or more) identity to SEQ ID NO: 366. In some embodiments, the antibody comprises a variable heavy chain (VH) region having an amino acid sequence 100% identical to the amino acid sequence of SEQ ID NO: 362 and a variable light chain (VL) region having an amino acid sequence 100% identical to the amino acid sequence of SEQ ID NO: 366. In some embodiments, the antibody comprises a VH comprising therein a complementarity determining region (CDR) H1 having an amino acid sequence 100% identical to the amino acid sequence of SEQ ID NO: 363, a CDR H2 having an amino acid sequence 100% identical to the amino acid sequence of SEQ ID NO: 364, and a CDR H3 having an amino acid sequence 100% identical to the amino acid sequence of SEQ ID NO: 365; and a VL comprising therein a complementarity determining region (CDR) L1 having an amino acid sequence 100% identical to the amino acid sequence of SEQ ID NO: 367, a CDR L2 having an amino acid sequence 100% identical to the amino acid sequence of AAS, and a CDR L3 having an amino acid sequence 100% identical to the amino acid sequence of SEQ ID NO: 368.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 369 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 373. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 369 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 373. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 369 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 373. In some embodiments, the antibody comprises a variable heavy chain (VH) region having an amino acid sequence with at least 95% (e.g., at least 95%, 96%, 97%, 98%, 99%, or more) identity to SEQ ID NO: 369 and a variable light chain (VL) region having an amino acid sequence with at least 95% (e.g., at least 95%, 96%, 97%, 98%, 99%, or more) identity to SEQ ID NO: 373. In some embodiments, the antibody comprises a variable heavy chain (VH) region having an amino acid sequence 100% identical to the amino acid sequence of SEQ ID NO: 369 and a variable light chain (VL) region having an amino acid sequence 100% identical to the amino acid sequence of SEQ ID NO: 373. In some embodiments, the antibody comprises a VH comprising therein a complementarity determining region (CDR) H1 having an amino acid sequence 100% identical to the amino acid sequence of SEQ ID NO: 370, a CDR H2 having an amino acid sequence 100% identical to the amino acid sequence of SEQ ID NO: 371, and a CDR H3 having an amino acid sequence 100% identical to the amino acid sequence of SEQ ID NO: 372; and a VL comprising therein a complementarity determining region (CDR) L1 having an amino acid sequence 100% identical to the amino acid sequence of SEQ ID NO: 374, a CDR L2 having an amino acid sequence 100% identical to the amino acid sequence of GTS, and a CDR L3 having an amino acid sequence 100% identical to the amino acid sequence of SEQ ID NO: 375.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 376 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 380. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 376 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 380. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 376 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 380. In some embodiments, the antibody comprises a variable heavy chain (VH) region having an amino acid sequence with at least 95% (e.g., at least 95%, 96%, 97%, 98%, 99%, or more) identity to SEQ ID NO: 376 and a variable light chain (VL) region having an amino acid sequence with at least 95% (e.g., at least 95%, 96%, 97%, 98%, 99%, or more) identity to SEQ ID NO: 380. In some embodiments, the antibody comprises a variable heavy chain (VH) region having an amino acid sequence 100% identical to the amino acid sequence of SEQ ID NO: 376 and a variable light chain (VL) region having an amino acid sequence 100% identical to the amino acid sequence of SEQ ID NO: 380. In some embodiments, the antibody comprises a VH comprising therein a complementarity determining region (CDR) H1 having an amino acid sequence 100% identical to the amino acid sequence of SEQ ID NO: 377, a CDR H2 having an amino acid sequence 100% identical to the amino acid sequence of SEQ ID NO: 378, and a CDR H3 having an amino acid sequence 100% identical to the amino acid sequence of SEQ ID NO: 379; and a VL comprising therein a complementarity determining region (CDR) L1 having an amino acid sequence 100% identical to the amino acid sequence of SEQ ID NO: 381, a CDR L2 having an amino acid sequence 100% identical to the amino acid sequence of ETS, and a CDR L3 having an amino acid sequence 100% identical to the amino acid sequence of SEQ ID NO: 382.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 383 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 387. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 383 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 387. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 383 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 387. In some embodiments, the antibody comprises a variable heavy chain (VH) region having an amino acid sequence with at least 95% (e.g., at least 95%, 96%, 97%, 98%, 99%, or more) identity to SEQ ID NO: 383 and a variable light chain (VL) region having an amino acid sequence with at least 95% (e.g., at least 95%, 96%, 97%, 98%, 99%, or more) identity to SEQ ID NO: 387. In some embodiments, the antibody comprises a variable heavy chain (VH) region having an amino acid sequence 100% identical to the amino acid sequence of SEQ ID NO: 383 and a variable light chain (VL) region having an amino acid sequence 100% identical to the amino acid sequence of SEQ ID NO: 387. In some embodiments, the antibody comprises a VH comprising therein a complementarity determining region (CDR) H1 having an amino acid sequence 100% identical to the amino acid sequence of SEQ ID NO: 384, a CDR H2 having an amino acid sequence 100% identical to the amino acid sequence of SEQ ID NO: 385, and a CDR H3 having an amino acid sequence 100% identical to the amino acid sequence of SEQ ID NO: 386; and a VL comprising therein a complementarity determining region (CDR) L1 having an amino acid sequence 100% identical to the amino acid sequence of SEQ ID NO: 388, a CDR L2 having an amino acid sequence 100% identical to the amino acid sequence of GAS, and a CDR L3 having an amino acid sequence 100% identical to the amino acid sequence of SEQ ID NO: 389.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 390 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 394. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 390 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 394. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 390 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 394. In some embodiments, the antibody comprises a variable heavy chain (VH) region having an amino acid sequence with at least 95% (e.g., at least 95%, 96%, 97%, 98%, 99%, or more) identity to SEQ ID NO: 390 and a variable light chain (VL) region having an amino acid sequence with at least 95% (e.g., at least 95%, 96%, 97%, 98%, 99%, or more) identity to SEQ ID NO: 394. In some embodiments, the antibody comprises a variable heavy chain (VH) region having an amino acid sequence 100% identical to the amino acid sequence of SEQ ID NO: 390 and a variable light chain (VL) region having an amino acid sequence 100% identical to the amino acid sequence of SEQ ID NO: 394. In some embodiments, the antibody comprises a VH comprising therein a complementarity determining region (CDR) H1 having an amino acid sequence 100% identical to the amino acid sequence of SEQ ID NO: 391, a CDR H2 having an amino acid sequence 100% identical to the amino acid sequence of SEQ ID NO: 392, and a CDR H3 having an amino acid sequence 100% identical to the amino acid sequence of SEQ ID NO: 393; and a VL comprising therein a complementarity determining region (CDR) L1 having an amino acid sequence 100% identical to the amino acid sequence of SEQ ID NO: 395, a CDR L2 having an amino acid sequence 100% identical to the amino acid sequence of DAS, and a CDR L3 having an amino acid sequence 100% identical to the amino acid sequence of SEQ ID NO: 396. In some embodiments, the antibody comprises a first heavy chain (HC) region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 462, a second heavy chain region comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 482, and a light chain (LC) region, comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 472.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 397 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 401. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 397 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 401. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 397 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 401. In some embodiments, the antibody comprises a variable heavy chain (VH) region having an amino acid sequence with at least 95% (e.g., at least 95%, 96%, 97%, 98%, 99%, or more) identity to SEQ ID NO: 397 and a variable light chain (VL) region having an amino acid sequence with at least 95% (e.g., at least 95%, 96%, 97%, 98%, 99%, or more) identity to SEQ ID NO: 401. In some embodiments, the antibody comprises a variable heavy chain (VH) region having an amino acid sequence 100% identical to the amino acid sequence of SEQ ID NO: 397 and a variable light chain (VL) region having an amino acid sequence 100% identical to the amino acid sequence of SEQ ID NO: 401. In some embodiments, the antibody comprises a VH comprising therein a complementarity determining region (CDR) H1 having an amino acid sequence 100% identical to the amino acid sequence of SEQ ID NO: 398, a CDR H2 having an amino acid sequence 100% identical to the amino acid sequence of SEQ ID NO: 399, and a CDR H3 having an amino acid sequence 100% identical to the amino acid sequence of SEQ ID NO: 400; and a VL comprising therein a complementarity determining region (CDR) L1 having an amino acid sequence 100% identical to the amino acid sequence of SEQ ID NO: 402, a CDR L2 having an amino acid sequence 100% identical to the amino acid sequence of GAS, and a CDR L3 having an amino acid sequence 100% identical to the amino acid sequence of SEQ ID NO: 403.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 404 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 408. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 404 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 408. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 404 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 408. In some embodiments, the antibody comprises a variable heavy chain (VH) region having an amino acid sequence with at least 95% (e.g., at least 95%, 96%, 97%, 98%, 99%, or more) identity to SEQ ID NO: 404 and a variable light chain (VL) region having an amino acid sequence with at least 95% (e.g., at least 95%, 96%, 97%, 98%, 99%, or more) identity to SEQ ID NO: 408. In some embodiments, the antibody comprises a variable heavy chain (VH) region having an amino acid sequence 100% identical to the amino acid sequence of SEQ ID NO: 404 and a variable light chain (VL) region having an amino acid sequence 100% identical to the amino acid sequence of SEQ ID NO: 408. In some embodiments, the antibody comprises a VH comprising therein a complementarity determining region (CDR) H1 having an amino acid sequence 100% identical to the amino acid sequence of SEQ ID NO: 405, a CDR H2 having an amino acid sequence 100% identical to the amino acid sequence of SEQ ID NO: 406, and a CDR H3 having an amino acid sequence 100% identical to the amino acid sequence of SEQ ID NO: 407; and a VL comprising therein a complementarity determining region (CDR) L1 having an amino acid sequence 100% identical to the amino acid sequence of SEQ ID NO: 409, a CDR L2 having an amino acid sequence 100% identical to the amino acid sequence of DAS, and a CDR L3 having an amino acid sequence 100% identical to the amino acid sequence of SEQ ID NO: 410.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 411 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 415. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 411 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 415. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 411 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 415. In some embodiments, the antibody comprises a variable heavy chain (VH) region having an amino acid sequence with at least 95% (e.g., at least 95%, 96%, 97%, 98%, 99%, or more) identity to SEQ ID NO: 411 and a variable light chain (VL) region having an amino acid sequence with at least 95% (e.g., at least 95%, 96%, 97%, 98%, 99%, or more) identity to SEQ ID NO: 415. In some embodiments, the antibody comprises a variable heavy chain (VH) region having an amino acid sequence 100% identical to the amino acid sequence of SEQ ID NO: 411 and a variable light chain (VL) region having an amino acid sequence 100% identical to the amino acid sequence of SEQ ID NO: 415. In some embodiments, the antibody comprises a VH comprising therein a complementarity determining region (CDR) H1 having an amino acid sequence 100% identical to the amino acid sequence of SEQ ID NO: 412, a CDR H2 having an amino acid sequence 100% identical to the amino acid sequence of SEQ ID NO: 413, and a CDR H3 having an amino acid sequence 100% identical to the amino acid sequence of SEQ ID NO: 414; and a VL comprising therein a complementarity determining region (CDR) L1 having an amino acid sequence 100% identical to the amino acid sequence of SEQ ID NO: 416, a CDR L2 having an amino acid sequence 100% identical to the amino acid sequence of DAF, and a CDR L3 having an amino acid sequence 100% identical to the amino acid sequence of SEQ ID NO: 417.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 418 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 422. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 418 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 422. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 418 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 422. In some embodiments, the antibody comprises a variable heavy chain (VH) region having an amino acid sequence with at least 95% (e.g., at least 95%, 96%, 97%, 98%, 99%, or more) identity to SEQ ID NO: 418 and a variable light chain (VL) region having an amino acid sequence with at least 95% (e.g., at least 95%, 96%, 97%, 98%, 99%, or more) identity to SEQ ID NO: 422. In some embodiments, the antibody comprises a variable heavy chain (VH) region having an amino acid sequence 100% identical to the amino acid sequence of SEQ ID NO: 418 and a variable light chain (VL) region having an amino acid sequence 100% identical to the amino acid sequence of SEQ ID NO: 422. In some embodiments, the antibody comprises a VH comprising therein a complementarity determining region (CDR) H1 having an amino acid sequence 100% identical to the amino acid sequence of SEQ ID NO: 419, a CDR H2 having an amino acid sequence 100% identical to the amino acid sequence of SEQ ID NO: 420, and a CDR H3 having an amino acid sequence 100% identical to the amino acid sequence of SEQ ID NO: 421; and a VL comprising therein a complementarity determining region (CDR) L1 having an amino acid sequence 100% identical to the amino acid sequence of SEQ ID NO: 423, a CDR L2 having an amino acid sequence 100% identical to the amino acid sequence of DAS, and a CDR L3 having an amino acid sequence 100% identical to the amino acid sequence of SEQ ID NO: 424.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 425 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 429. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 425 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 429. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 425 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 429. In some embodiments, the antibody comprises a variable heavy chain (VH) region having an amino acid sequence with at least 95% (e.g., at least 95%, 96%, 97%, 98%, 99%, or more) identity to SEQ ID NO: 425 and a variable light chain (VL) region having an amino acid sequence with at least 95% (e.g., at least 95%, 96%, 97%, 98%, 99%, or more) identity to SEQ ID NO: 429. In some embodiments, the antibody comprises a variable heavy chain (VH) region having an amino acid sequence 100% identical to the amino acid sequence of SEQ ID NO: 425 and a variable light chain (VL) region having an amino acid sequence 100% identical to the amino acid sequence of SEQ ID NO: 429. In some embodiments, the antibody comprises a VH comprising therein a complementarity determining region (CDR) H1 having an amino acid sequence 100% identical to the amino acid sequence of SEQ ID NO: 426, a CDR H2 having an amino acid sequence 100% identical to the amino acid sequence of SEQ ID NO: 427, and a CDR H3 having an amino acid sequence 100% identical to the amino acid sequence of SEQ ID NO: 428; and a VL comprising therein a complementarity determining region (CDR) L1 having an amino acid sequence 100% identical to the amino acid sequence of SEQ ID NO: 430, a CDR L2 having an amino acid sequence 100% identical to the amino acid sequence of DAS, and a CDR L3 having an amino acid sequence 100% identical to the amino acid sequence of SEQ ID NO: 431.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 432 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 436. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 432 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 436. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 432 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 436. In some embodiments, the antibody comprises a variable heavy chain (VH) region having an amino acid sequence with at least 95% (e.g., at least 95%, 96%, 97%, 98%, 99%, or more) identity to SEQ ID NO: 432 and a variable light chain (VL) region having an amino acid sequence with at least 95% (e.g., at least 95%, 96%, 97%, 98%, 99%, or more) identity to SEQ ID NO: 436. In some embodiments, the antibody comprises a variable heavy chain (VH) region having an amino acid sequence 100% identical to the amino acid sequence of SEQ ID NO: 432 and a variable light chain (VL) region having an amino acid sequence 100% identical to the amino acid sequence of SEQ ID NO: 436. In some embodiments, the antibody comprises a VH comprising therein a complementarity determining region (CDR) H1 having an amino acid sequence 100% identical to the amino acid sequence of SEQ ID NO: 433, a CDR H2 having an amino acid sequence 100% identical to the amino acid sequence of SEQ ID NO: 434, and a CDR H3 having an amino acid sequence 100% identical to the amino acid sequence of SEQ ID NO: 435; and a VL comprising therein a complementarity determining region (CDR) L1 having an amino acid sequence 100% identical to the amino acid sequence of SEQ ID NO: 437, a CDR L2 having an amino acid sequence 100% identical to the amino acid sequence of GAS, and a CDR L3 having an amino acid sequence 100% identical to the amino acid sequence of SEQ ID NO: 438.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 439 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 443. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 439 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 443. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 439 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 443. In some embodiments, the antibody comprises a variable heavy chain (VH) region having an amino acid sequence with at least 95% (e.g., at least 95%, 96%, 97%, 98%, 99%, or more) identity to SEQ ID NO: 439 and a variable light chain (VL) region having an amino acid sequence with at least 95% (e.g., at least 95%, 96%, 97%, 98%, 99%, or more) identity to SEQ ID NO: 443. In some embodiments, the antibody comprises a variable heavy chain (VH) region having an amino acid sequence 100% identical to the amino acid sequence of SEQ ID NO: 439 and a variable light chain (VL) region having an amino acid sequence 100% identical to the amino acid sequence of SEQ ID NO: 443. In some embodiments, the antibody comprises a VH comprising therein a complementarity determining region (CDR) H1 having an amino acid sequence 100% identical to the amino acid sequence of SEQ ID NO: 440, a CDR H2 having an amino acid sequence 100% identical to the amino acid sequence of SEQ ID NO: 441, and a CDR H3 having an amino acid sequence 100% identical to the amino acid sequence of SEQ ID NO: 442; and a VL comprising therein a complementarity determining region (CDR) L1 having an amino acid sequence 100% identical to the amino acid sequence of SEQ ID NO: 444, a CDR L2 having an amino acid sequence 100% identical to the amino acid sequence of AAS, and a CDR L3 having an amino acid sequence 100% identical to the amino acid sequence of SEQ ID NO: 445.

In some embodiments, the antibody comprises a variable heavy chain (VH) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 446 and a variable light chain (VL) region having an amino acid sequence with at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 450. In some embodiments, the antibody comprises a variable heavy chain (VH) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 446 and a variable light chain (VL) region having an amino acid sequence with at least 85% (e.g., at least 85%, 86%, 87%, 88%, 89%, 90%, 91%, 92%, 93%, 94%, 95%, 96%, 97%, 98%, 99%, or more) identity to SEQ ID NO: 450. In some embodiments, the antibody comprises a variable heavy chain (VH) region having an amino acid sequence with at least 90% (e.g., at least 90%, 91%, 92%, 93%, 94%, 95%, 96%, 97%, 98%, 99%, or more) identity to SEQ ID NO: 446 and a variable light chain (VL) region having an amino acid sequence with at least 90% (e.g., at least 90%, 91%, 92%, 93%, 94%, 95%, 96%, 97%, 98%, 99%, or more) identity to SEQ ID NO: 450. In some embodiments, the antibody comprises a variable heavy chain (VH) region having an amino acid sequence with at least 95% (e.g., at least 95%, 96%, 97%, 98%, 99%, or more) identity to SEQ ID NO: 446 and a variable light chain (VL) region having an amino acid sequence with at least 95% (e.g., at least 95%, 96%, 97%, 98%, 99%, or more) identity to SEQ ID NO: 450. In some embodiments, the antibody comprises a variable heavy chain (VH) region having an amino acid sequence 100% identical to the amino acid sequence of SEQ ID NO: 446 and a variable light chain (VL) region having an amino acid sequence 100% identical to the amino acid sequence of SEQ ID NO: 450. In some embodiments, the antibody comprises a VH comprising therein a complementarity determining region (CDR) H1 having an amino acid sequence 100% identical to the amino acid sequence of SEQ ID NO: 447, a CDR H2 having an amino acid sequence 100% identical to the amino acid sequence of SEQ ID NO: 448, and a CDR H3 having an amino acid sequence 100% identical to the amino acid sequence of SEQ ID NO: 449; and a VL comprising therein a complementarity determining region (CDR) L1 having an amino acid sequence 100% identical to the amino acid sequence of SEQ ID NO: 451, a CDR L2 having an amino acid sequence 100% identical to the amino acid sequence of DNF, and a CDR L3 having an amino acid sequence 100% identical to the amino acid sequence of SEQ ID NO: 452.

In some embodiments, an antibody of the disclosure is an antibody selected from Table 1. In some embodiments, an antibody of the disclosure is antibody Lib1_P1_A9 of Table 1. In some embodiments, an antibody of the disclosure is antibody Lib1_G3_A9 of Table 1. In some embodiments, an antibody of the disclosure is antibody 1675_Lib1 of Table 1. In some embodiments, an antibody of the disclosure is antibody 7046_Lib8 of Table 1. In some embodiments, an antibody of the disclosure is antibody Lib1_P1_A6 of Table 1. In some embodiments, an antibody of the disclosure is antibody 5802_Lib4 of Table 1. In some embodiments, an antibody of the disclosure is antibody 1374_Lib8 of Table 1. In some embodiments, an antibody of the disclosure is antibody 9301_Lib8 of Table 1. In some embodiments, an antibody of the disclosure is antibody 9621_Lib8 of Table 1. In some embodiments, an antibody of the disclosure is antibody 1405_Lib8 of Table 1. In some embodiments, an antibody of the disclosure is antibody Lib4_P2_H3 of Table 1. In some embodiments, an antibody of the disclosure is antibody 1081_Lib8 of Table 1. In some embodiments, an antibody of the disclosure is antibody Lib4_P2_A5 of Table 1. In some embodiments, an antibody of the disclosure is antibody Lib1_P1_D1 of Table 1. In some embodiments, an antibody of the disclosure is antibody Lib1_P1_C6 of Table 1. In some embodiments, an antibody of the disclosure is antibody 4142_Lib1 of Table 1. In some embodiments, an antibody of the disclosure is antibody Lib1_P1_E9 of Table 1. In some embodiments, an antibody of the disclosure is antibody Lib8_P4_D4 of Table 1. In some embodiments, an antibody of the disclosure is antibody 4166_Lib4 of Table 1. In some embodiments, an antibody of the disclosure is antibody Lib4_P2_B4 of Table 1. In some embodiments, an antibody of the disclosure is antibody Lib8_P4_C11 of Table 1. In some embodiments, an antibody of the disclosure is antibody 3964_Lib4 of Table 1. In some embodiments, an antibody of the disclosure is antibody 3552_Lib8 of Table 1. In some embodiments, an antibody of the disclosure is antibody 2738_Lib1 of Table 1. In some embodiments, an antibody of the disclosure is antibody Lib4_P2_A1 of Table 1. In some embodiments, an antibody of the disclosure is antibody 189_Lib4 of Table 1. In some embodiments, an antibody of the disclosure is antibody Lib1_P1_A1 of Table 1. In some embodiments, an antibody of the disclosure is antibody Lib6_P3_B9 of Table 1. In some embodiments, an antibody of the disclosure is antibody Lib6_P3_A1 of Table 1. In some embodiments, an antibody of the disclosure is antibody Lib4_P2_A3 of Table 1. In some embodiments, an antibody of the disclosure is antibody 487_Lib1 of Table 1. In some embodiments, an antibody of the disclosure is antibody Lib6_P3_E10 of Table 1. In some embodiments, an antibody of the disclosure is antibody 256_Lib8 of Table 1. In some embodiments, an antibody of the disclosure is antibody 4035_Lib1 of Table 1. In some embodiments, an antibody of the disclosure is antibody 96_Lib4 of Table 1. In some embodiments, an antibody of the disclosure is antibody Lib1_P1_A2 of Table 1. In some embodiments, an antibody of the disclosure is antibody 2301_Lib4 of Table 1. In some embodiments, an antibody of the disclosure is antibody Lib4_P2_B1 of Table 1. In some embodiments, an antibody of the disclosure is antibody Lib4_P2_A2 of Table 1. In some embodiments, an antibody of the disclosure is antibody 16_Lib8 of Table 1. In some embodiments, an antibody of the disclosure is antibody 11485_Lib1 of Table 1. In some embodiments, an antibody of the disclosure is antibody 4956_Lib1 of Table 1. In some embodiments, an antibody of the disclosure is antibody 399_Lib1 of Table 1. In some embodiments, an antibody of the disclosure is antibody Lib4_P2_A10 of Table 1. In some embodiments, an antibody of the disclosure is antibody Lib6_P3_A8 of Table 1. In some embodiments, an antibody of the disclosure is antibody 965_Lib4 of Table 1. In some embodiments, an antibody of the disclosure is antibody 27_Lib4 of Table 1. In some embodiments, an antibody of the disclosure is antibody 15244_Lib8 of Table 1. In some embodiments, an antibody of the disclosure is antibody Lib4_P2_A11 of Table 1. In some embodiments, an antibody of the disclosure is antibody Lib8_P4_C10 of Table 1. In some embodiments, an antibody of the disclosure is antibody 8226_Lib8 of Table 1. In some embodiments, an antibody of the disclosure is antibody 69_Lib8 of Table 1. In some embodiments, an antibody of the disclosure is antibody 76_Lib4 of Table 1. In some embodiments, an antibody of the disclosure is antibody 4675_Lib4 of Table 1. In some embodiments, an antibody of the disclosure is antibody 4029_Lib1 of Table 1. In some embodiments, an antibody of the disclosure is antibody 5662_Lib8 of Table 1. In some embodiments, an antibody of the disclosure is antibody Lib6_P3_A4 of Table 1. In some embodiments, an antibody of the disclosure is antibody Lib1_P1_F10 of Table 1. In some embodiments, an antibody of the disclosure is antibody Lib4_P2_D1 of Table 1. In some embodiments, an antibody of the disclosure is antibody Lib1_P1_A5 of Table 1. In some embodiments, an antibody of the disclosure is antibody 9312_Lib8 of Table 1. In some embodiments, an antibody of the disclosure is antibody 4761_Lib8 of Table 1. In some embodiments, an antibody of the disclosure is antibody 17561_Lib6 of Table 1.

TABLE 1
Sequences of VH and VL regions of identified anti-VWF CK domain
antibodies

| | | | SEQ | |
|---|---|---|---|---|
| Antibody ID | Domain | Segment | ID NO | Amino Acid Sequence |
| Lib1_P1_A9 | VH | All | 12 | QVQLVQSGAEVKKPGASVKVSCKVS |
| | | | | GDTLSSYGIHWVRQAPGKGLEWMGG |
| | | | | SIPSFGTAIYAQKFQGRVTMTEDTSTD |
| | | | | TAYMELSSLKSEDTAVYYCTSEGPWL |
| | | | | PFFSFDYWGQGTLVTVSS |
| | | CDR H1 | 13 | GDTLSSYG |
| | | CDR H2 | 14 | SIPSFGTA |
| | | CDR H3 | 15 | TSEGPWLPFFSFDY |
| | VL | All | 16 | DIQMTQSPSSVSASVGDRVTITCRASQ |
| | | | | GISRNLAWYQQKPGKAPKLLIYKASS |
| | | | | LASGVPSRFSGSGSGTDFTLTISSLQPE |
| | | | | DFANYYCQQSFSTPVTFGGGTKVEIK |
| | | CDR L1 | 17 | QGISRN |
| | | CDR L2 | | KAS |
| | | CDR L3 | 18 | QQSFSTPVT |
| Lib1_P1_G3 | VH | All | 19 | QVQLVQSGAEVKKPGASVKVSCKVS |
| | | | | GYTSRQYIHWVRQAPGKGLEWMGW |
| | | | | VTPIFGTTNYAQKFQGRVTMTEDTST |
| | | | | DTAYMELSSLKSEDTAVYYCAREWT |
| | | | | GTTAFDIWGQGTLVTVSS |
| | | CDR H1 | 20 | GYTSRQY |
| | | CDR H2 | 21 | VTPIFGTT |
| | | CDR H3 | 22 | AREWTGTTAFDI |
| | VL | All | 23 | DIQMTQSPSSVSASVGDRVTITCRASQ |
| | | | | DIGHYLAWYQQKPGKAPKLLIYAASN |
| | | | | RASGVPSRFSGSGSGTDFTLTISSLQPE |
| | | | | DFANYYCQQGYSAPYGFGGGTKVEIK |
| | | CDR L1 | 24 | QDIGHY |
| | | CDR L2 | | AAS |
| | | CDR L3 | 25 | QQGYSAPYG |
| 1675_Lib1 | VH | All | 26 | QVQLVQSGAEVKKPGASVKVSCKVS |
| | | | | GVTLTSSAISWVRQAPGKGLEWMGGI |
| | | | | TPIFGTPIYAQKFQGRVTMTEDTSTDT |
| | | | | AYMELSSLKSEDTAVYYCATDYGYPF |
| | | | | WAFDIWGQGTMVTVSS |
| | | CDR H1 | 27 | GVTLTSSA |
| | | CDR H2 | 28 | ITPIFGTP |
| | | CDR H3 | 29 | ATDYGYPFWAFDI |
| | VL | All | 30 | DIQMTQSPSSVSASVGDRVTITCRASQ |
| | | | | GIYSALAWYQQKPGKAPKLLIYESSK |
| | | | | RPSGVPSRFSGSGSGTDFTLTISSLQPE |
| | | | | DFANYYCEQYASSPPTFGGGTKVEIK |
| | | CDR L1 | 31 | QGIYSA |
| | | CDR L2 | | ESS |
| | | CDR L3 | 32 | EQYASSPPT |
| 7046_Lib8 | VH | All | 33 | EVQLVESGGGLVQPGRSLRLSCAASG |
| | | | | FNFHNYDMHWVRQAPGKGLEWVSAI |
| | | | | SGSGYTADYADSVEGRFTISRDNAKN |
| | | | | SLYLQMNSLRAEDTAVYYCAKSHSSG |
| | | | | WSSAFDIWGQGTMVTVSS |
| | | CDR H1 | 34 | GFNFHNYD |
| | | CDR H2 | 35 | ISGSGYTA |
| | | CDR H3 | 36 | AKSHSSGWSSAFDI |
| | VL | All | 37 | DIQMTQSPSSLSASVGDRVTITCRASQ |
| | | | | TIGYWLAWYQQKPGKAPKLLIYDAT |
| | | | | NLATGVPSRFSGSGSGTDFTLTISSLQP |
| | | | | EDVATYYCQHYNPYSQVTFGGGTKV |
| | | | | EIK |
| | | CDR L1 | 38 | QTIGYW |
| | | CDR L2 | | DAT |
| | | CDR L3 | 39 | QHYNPYSQVT |
| Lib1_P1_A6 | VH | All | 40 | QVQLVQSGAEVKKPGASVKVSCKVS |
| | | | | GSTFSNYAIHWVRQAPGKGLEWMGG |
| | | | | IVPLSGTAIYAQKFQGRVTMTEDTSTD |
| | | | | TAYMELSSLKSEDTAVYYCARDQGES |
| | | | | SGWYAIYYGMDVWGQGTTVTVSS |
| | | CDR H1 | 41 | GSTFSNYA |
| | | CDR H2 | 42 | IVPLSGTA |
| | | CDR H3 | 43 | ARDQGESSGWYAIYYGMDV |
| | VL | All | 44 | DIQMTQSPSSVSASVGDRVTITCRASQ |
| | | | | GIGTWLAWYQQKPGKAPKLLIYGSSS |
| | | | | RASGVPSRFSGSGSGTDFTLTISSLQPE |
| | | | | DFANYYCQQSYGKPTFGGGTKVEIK |
| | | CDR L1 | 45 | QGIGTW |
| | | CDR L2 | | GSS |
| | | CDR L3 | 46 | QQSYGKPT |
| 5802_Lib4 | VH | All | 47 | QVQLQESGPGLVKPSQTLSLTCTVSG |
| | | | | YSISSGDYHWSWIRQPPGKGLEWIGYI |
| | | | | NPSGSADYNPSLKSRVTMSVDTSKNQ |
| | | | | FSLKVNSVTAADTAVYYCARDGVAA |
| | | | | TYYYYYGMDVWGQGTTVTVSS |
| | | CDR H1 | 48 | GYSISSGDYH |
| | | CDR H2 | 49 | INPSGSA |
| | | CDR H3 | 50 | ARDGVAATYYYYYGMDV |
| | VL | All | 51 | EIVMTQSPATLSLSPGERATLSCRASH |
| | | | | SIGSNLAWYQQKPGQAPRLLIYDATT |
| | | | | RATGIPARFSGSGSGTDFTLTISSLEPE |
| | | | | DFAVYYCQQAYSFPLTFGGGTKVEIK |
| | | CDR L1 | 52 | HSIGSN |
| | | CDR L2 | | DAT |
| | | CDR L3 | 53 | QQAYSFPLT |
| 1374_Lib8 | VH | All | 54 | EVQLVESGGGLVQPGRSLRLSCAASG |
| | | | | VDFSTYAMHWVRQAPGKGLEWVSAI |
| | | | | SSSGAYTDYADSVEGRFTISRDNAKN |
| | | | | SLYLQMNSLRAEDTAVYYCATDRGPF |
| | | | | SGYGTQGWFDPWGQGTLVTVSS |
| | | CDR H1 | 55 | GVDFSTYA |
| | | CDR H2 | 56 | ISSSGAYT |
| | | CDR H3 | 57 | ATDRGPFSGYGTQGWFDP |
| | VL | All | 58 | DIQMTQSPSSLSASVGDRVTITCRASQ |
| | | | | TIRSYLAWYQQKPGKAPKLLIYGASD |
| | | | | LQSGVPSRFSGSGSGTDFTLTISSLQPE |
| | | | | DVATYYCQQYANFPITFGGGTKVEIK |
| | | CDR L1 | 59 | QTIRSY |
| | | CDR L2 | | GAS |
| | | CDR L3 | 60 | QQYANFPIT |
| 9301_Lib8 | VH | All | 61 | EVQLVESGGGLVQPGRSLRLSCAASG |
| | | | | FNFHNYDMHWVRQAPGKGLEWVSAI |
| | | | | SGSGYTADYADSVEGRFTISRDNAKN |
| | | | | SLYLQMNSLRAEDTAVYYCAKSHSSG |
| | | | | WSSAFDIWGQGTMVTVSS |
| | | CDR H1 | 62 | GFNFHNYD |
| | | CDR H2 | 63 | ISGSGYTA |
| | | CDR H3 | 64 | AKSHSSGWSSAFDI |
| | VL | All | 65 | DIQMTQSPSSLSASVGDRVTITCRASQ |
| | | | | TIGYWLAWYQQKPGKAPKLLIYDAS |
| | | | | VPESGVPSRFSGSGSGTDFTLTISSLQP |
| | | | | EDVATYYCQQTLSSPWTFGGGTKVEI |
| | | | | K |
| | | CDR L1 | 66 | QTIGYW |
| | | CDR L2 | | DAS |
| | | CDR L3 | 67 | QQTLSSPWT |
| 9621_Lib8 | VH | All | 68 | EVQLVESGGGLVQPGRSLRLSCAASG |
| | | | | FSFSMYAMHWVRQAPGKGLEWVSAI |
| | | | | SPSGDATDYADSVEGRFTISRDNAKN |
| | | | | SLYLQMNSLRAEDTAVYYCARGMQD |
| | | | | YGHYYYYYMDVWGKGTTVTVSS |
| | | CDR H1 | 69 | GFSFSMYA |
| | | CDR H2 | 70 | ISPSGDAT |
| | | CDR H3 | 71 | ARGMQDYGHYYYYYMDV |
| | VL | All | 72 | DIQMTQSPSSLSASVGDRVTITCRASQ |
| | | | | DISTSLAWYQQKPGKAPKLLIYGASSL |
| | | | | ETGVPSRFSGSGSGTDFTLTISSLQPED |
| | | | | VATYYCQQGYIAPRTFGGGTKVEIK |
| | | CDR L1 | 73 | QDISTS |
| | | CDR L2 | | GAS |
| | | CDR L3 | 74 | QQGYIAPRT |
| 1405_Lib8 | VH | All | 75 | EVQLVESGGGLVQPGRSLRLSCAASG |
| | | | | VDFSTYAMHWVRQAPGKGLEWVSAI |
| | | | | SSSGAYTDYADSVEGRFTISRDNAKN |
| | | | | SLYLQMNSLRAEDTAVYYCATDRGPF |
| | | | | SGYGTQGWFDPWGQGTLVTVSS |
| | | CDR H1 | 76 | GVDFSTYA |
| | | CDR H2 | 77 | ISSSGAYT |
| | | CDR H3 | 78 | ATDRGPFSGYGTQGWFDP |
| | VL | All | 79 | DIQMTQSPSSLSASVGDRVTITCRASR |
| | | | | DITSYLAWYQQKPGKAPKLLIYHAST |
| | | | | LEAGVPSRFSGSGSGTDFTLTISSLQPE |
| | | | | DVATYYCQQYGDFPPTFGGGTKVEIK |
| | | CDR L1 | 80 | RDITSY |
| | | CDR L2 | | HAS |
| | | CDR L3 | 81 | QQYGDFPPT |
| Lib4_P2_H3 | VH | All | 82 | QVQLQESGPGLVKPSQTLSLTCTVSGS |
| | | | | TFGDYAWSWIRQPPGKGLEWIGYINH |
| | | | | IGNADYNPSLKSRVTMSVDTSKNQFS |
| | | | | LKVNSVTAADTAVYYCARDGVESPE |
| | | | | VPNYYYYGMDVWGQGTTVTVSS |
| | | CDR H1 | 83 | GSTFGDYA |
| | | CDR H2 | 84 | INHIGNA |
| | | CDR H3 | 85 | ARDGVESPEVPNYYYYGMDV |
| | VL | All | 86 | EIVMTQSPATLSLSPGERATLSCRASH |
| | | | | SIGSNLAWYQQKPGQAPRLLIYDATN |
| | | | | RAPGIPARFSGSGSGTDFTLTISSLEPE |
| | | | | DFAVYYCQQSYTNPTFGGGTKVEIK |
| | | CDR L1 | 87 | HSIGSN |
| | | CDR L2 | | DAT |
| | | CDR L3 | 88 | QQSYTNPT |
| 1081_Lib8 | VH | All | 89 | EVQLVESGGGLVQPGRSLRLSCAASG |
| | | | | FNFHNYDMHWVRQAPGKGLEWVSAI |
| | | | | SGGAVSTDYADSVEGRFTISRDNAKN |
| | | | | SLYLQMNSLRAEDTAVYYCARSSWY |
| | | | | GGQIDFWGQGTLVTVSS |
| | | CDR H1 | 90 | GFNFHNYD |
| | | CDR H2 | 91 | ISGGAVST |
| | | CDR H3 | 92 | ARSSWYGGQIDF |
| | VL | All | 93 | DIQMTQSPSSLSASVGDRVTITCRASE |
| | | | | SVSSWLAWYQQKPGKAPKLLIYGAST |
| | | | | LEGGVPSRFSGSGSGTDFTLTISSLQPE |
| | | | | DVATYYCQQAYRTPVTFGGGTKVEIK |
| | | CDR L1 | 94 | ESVSSW |
| | | CDR L2 | | GAS |
| | | CDR L3 | 95 | QQAYRTPVT |
| Lib4_P2_A5 | VH | All | 96 | QVQLQESGPGLVKPSQTLSLTCTVSGS |
| | | | | SISSSSDYWSWIRQPPGKGLEWIGYIDI |
| | | | | SGDADYNPSLKSRVTMSVDTSKNQFS |
| | | | | LKVNSVTAADTAVYYCARHLSSGWY |
| | | | | GTYYFDYWGQGTLVTVSS |
| | | CDR H1 | 97 | GSSISSSSDY |
| | | CDR H2 | 98 | IDISGDA |
| | | CDR H3 | 99 | ARHLSSGWYGTYYFDY |
| | VL | All | 100 | EIVMTQSPATLSLSPGERATLSCRASQ |
| | | | | NIHSNLAWYQQKPGQAPRLLIYDASS |
| | | | | RATGIPARFSGSGSGTDFTLTISSLEPE |
| | | | | DFAVYYCQQGYTKPLTFGGGTKVEIK |
| | | CDR L1 | 101 | QNIHSN |
| | | CDR L2 | | DAS |
| | | CDR L3 | 102 | QQGYTKPLT |
| Lib1_P1_D1 | VH | All | 103 | QVQLVQSGAEVKKPGASVKVSCKVS |
| | | | | GDTFTDRYIHWVRQAPGKGLEWMGG |
| | | | | INTIFGTAIYAQKFQGRVTMTEDTSTD |
| | | | | TAYMELSSLKSEDTAVYYCAREGATV |
| | | | | STNFDYWGQGTLVTVSS |
| | | CDR H1 | 104 | GDTFTDRY |
| | | CDR H2 | 105 | INTIFGTA |
| | | CDR H3 | 106 | AREGATVSTNFDY |
| | VL | All | 107 | DIQMTQSPSSVSASVGDRVTITCRASQ |
| | | | | DIRKSLAWYQQKPGKAPKLLIYDISNR |
| | | | | PSGVPSRFSGSGSGTDFTLTISSLQPED |
| | | | | FANYYCQQGYSAPYGFGGGTKVEIK |
| | | CDR L1 | 108 | QDIRKS |
| | | CDR L2 | | DIS |
| | | CDR L3 | 109 | QQGYSAPYG |
| Lib1_P1_C6 | VH | All | 110 | QVQLVQSGAEVKKPGASVKVSCKVS |
| | | | | GYTFASHYIHWVRQAPGKGLEWMGG |
| | | | | MDPATGQTIYAQKFQGRVTMTEDTST |
| | | | | DTAYMELSSLKSEDTAVYYCAKGDR |
| | | | | SSWYEVLSFQHWGQGTLVTVSS |
| | | CDR H1 | 111 | GYTFASHY |
| | | CDR H2 | 112 | MDPATGQT |
| | | CDR H3 | 113 | AKGDRSSWYEVLSFQH |
| | VL | All | 114 | DIQMTQSPSSVSASVGDRVTITCRASQ |
| | | | | GISTDLAWYQQKPGKAPKLLIYDASIL |
| | | | | QRGVPSRFSGSGSGTDFTLTISSLQPED |
| | | | | FANYYCQQVRTYPFTFGGGTKVEIK |
| | | CDR L1 | 115 | QGISTD |
| | | CDR L2 | | DAS |
| | | CDR L3 | 116 | QQVRTYPFT |
| 4142_Lib1 | VH | All | 117 | QVQLVQSGAEVKKPGASVKVSCKVS |
| | | | | GYPFTSSGIHWVRQAPGKGLEWMGG |
| | | | | VIPLFGTANYAQKFQGRVTMTEDTST |
| | | | | DTAYMELSSLKSEDTAVYYCARIRVS |
| | | | | SGWYRDYGMDVWGQGTTVTVSS |
| | | CDR H1 | 118 | GYPFTSSG |
| | | CDR H2 | 119 | VIPLFGTA |
| | | CDR H3 | 120 | ARIRVSSGWYRDYGMDV |
| | VL | All | 121 | DIQMTQSPSSVSASVGDRVTITCRASQ |
| | | | | DITNDLAWYQQKPGKAPKLLIYDASR |
| | | | | LQSGVPSRFSGSGSGTDFTLTISSLQPE |
| | | | | DFANYYCQQTFTTPLTFGGGTKVEIK |
| | | CDR L1 | 122 | QDITND |
| | | CDR L2 | | DAS |
| | | CDR L3 | 123 | QQTFTTPLT |
| Lib1_P1_E9 | VH | All | 124 | QVQLVQSGAEVKKPGASVKVSCKVS |
| | | | | GYTFTSHIIHWVRQAPGKGLEWMGG |
| | | | | MNPYSNKAIYAQKFQGRVTMTEDTST |
| | | | | DTAYMELSSLKSEDTAVYYCARDAS |
| | | | | DYGDYGYYYYYMDVWGKGTTVTVS |
| | | | | S |
| | | CDR H1 | 125 | GYTFTSHI |
| | | CDR H2 | 126 | MNPYSNKA |
| | | CDR H3 | 127 | ARDASDYGDYGYYYYYMDV |
| | VL | All | 128 | DIQMTQSPSSVSASVGDRVTITCRASQ |
| | | | | YIGNDLAWYQQKPGKAPKLLIYEASQ |
| | | | | RPSGVPSRFSGSGSGTDFTLTISSLQPE |
| | | | | DFANYYCQQQYNYPLTFGGGTKVEIK |
| | | CDR L1 | 129 | QYIGND |
| | | CDR L2 | | EAS |
| | | CDR L3 | 130 | QQQYNYPLT |
| Lib8_P4_D4 | VH | All | 131 | QVQLVQSGAEVKKPGASVKVSCKVS |
| | | | | GDTLSNYAIHWVRQAPGKGLEWMGG |
| | | | | INPNAGTTIYAQKFQGRVTMTEDTST |
| | | | | DTAYMELSSLKSEDTAVYYCARDSSS |
| | | | | WCPVGYYYYYMDVWGKGTTVTVSS |
| | | CDR H1 | 132 | GDTLSNYA |
| | | CDR H2 | 133 | INPNAGTT |
| | | CDR H3 | 134 | ARDSSSWCPVGYYYYYMDV |
| | VL | All | 135 | DIQMTQSPSSVSASVGDRVTITCRASQ |
| | | | | SIGDFLAWYQQKPGKAPKLLIYDASH |
| | | | | LQTGVPSRFSGSGSGTDFTLTISSLQPE |
| | | | | DFANYYCQQTSTIPPTFGGGTKVEIK |
| | | CDR L1 | 136 | QSIGDF |
| | | CDR L2 | | DAS |
| | | CDR L3 | 137 | QQTSTIPPT |
| 4166_Lib4 | VH | All | 138 | QVQLQESGPGLVKPSQTLSLTCTVSGE |
| | | | | SFSGHFWSWIRQPPGKGLEWIGYISQS |
| | | | | GRTDYNPSLKSRVTMSVDTSKNQFSL |
| | | | | KVNSVTAADTAVYYCARTLPRIQLWS |
| | | | | PRGAFDIWGQGTMVTVSS |
| | | CDR H1 | 139 | GESFSGHF |
| | | CDR H2 | 140 | ISQSGRT |
| | | CDR H3 | 141 | ARTLPRIQLWSPRGAFDI |
| | VL | All | 142 | EIVMTQSPATLSLSPGERATLSCRASQ |
| | | | | SLGRSLAWYQQKPGQAPRLLIYDASH |
| | | | | RATGIPARFSGSGSGTDFTLTISSLEPE |
| | | | | DFAVYYCQQYVSYPLTFGGGTKVEIK |
| | | CDR L1 | 143 | QSLGRS |
| | | CDR L2 | | DAS |
| | | CDR L3 | 144 | QQYVSYPLT |
| Lib4_P2_B4 | VH | All | 145 | QVQLQESGPGLVKPSQTLSLTCTVSG |
| | | | | ASLGDQYWSWIRQPPGKGLEWIGYIH |
| | | | | PSGDFDYNPSLKSRVTMSVDTSKNQF |
| | | | | SLKVNSVTAADTAVYYCARHYSSSW |
| | | | | YGTWYFDLWGRGTLVTVSS |
| | | CDR H1 | 146 | GASLGDQY |
| | | CDR H2 | 147 | IHPSGDF |
| | | CDR H3 | 148 | ARHYSSSWYGTWYFDL |
| | VL | All | 149 | EIVMTQSPATLSLSPGERATLSCRASQ |
| | | | | NIGTNLAWYQQKPGQAPRLLIYDASV |
| | | | | RAAGIPARFSGSGSGTDFTLTISSLEPE |
| | | | | DFAVYYCQQSLTTPLTFGGGTKVEIK |
| | | CDR L1 | 150 | QNIGTN |
| | | CDR L2 | | DAS |
| | | CDR L3 | 151 | QQSLTTPLT |
| Lib8_P4_C11 | VH | All | 152 | QVQLVQSGAEVKKPGASVKVSCKVS |
| | | | | GDTLSNYAIHWVRQAPGKGLEWMGG |
| | | | | INPNAGTTIYAQKFQGRVTMTEDTST |
| | | | | DTAYMELSSLKSEDTAVYYCARDSSS |
| | | | | WCPVGYYYYYMDVWGKGTTVTVSS |
| | | CDR H1 | 153 | GDTLSNYA |
| | | CDR H2 | 154 | INPNAGTT |
| | | CDR H3 | 155 | ARDSSSWCPVGYYYYYMDV |
| | VL | All | 156 | DIQMTQSPSSVSASVGDRVTITCRASQ |
| | | | | SIGDFLAWYQQKPGKAPKLLIYDASH |
| | | | | LQTGVPSRFSGSGSGTDFTLTISSLQPE |
| | | | | DFANYYCQQTSTIPPTFGGGTKVEIK |
| | | CDR L1 | 157 | QSIGDF |
| | | CDR L2 | | DAS |
| | | CDR L3 | 158 | QQTSTIPPT |
| 3964_Lib4 | VH | All | 159 | QVQLQESGPGLVKPSQTLSLTCTVSG |
| | | | | ASISTHSWSWIRQPPGKGLEWIGYINP |
| | | | | SGDIDYNPSLKSRVTMSVDTSKNQFSL |
| | | | | KVNSVTAADTAVYYCARGRTGFDSS |
| | | | | GFVVGAFDIWGQGTMVTVSS |
| | | CDR H1 | 160 | GASISTHS |
| | | CDR H2 | 161 | INPSGDI |
| | | CDR H3 | 162 | ARGRTGFDSSGFVVGAFDI |
| | VL | All | 163 | EIVMTQSPATLSLSPGERATLSCRASQ |
| | | | | NVNDNLAWYQQKPGQAPRLLIYDAS |
| | | | | TRPTGIPARFSGSGSGTDFTLTISSLEPE |
| | | | | DFAVYYCQQYLTIPRTFGGGTKVEIK |
| | | CDR L1 | 164 | QNVNDN |
| | | CDR L2 | | DAS |
| | | CDR L3 | 165 | QQYLTIPRT |
| 3552_Lib8 | VH | All | 166 | EVQLVESGGGLVQPGRSLRLSCAASE |
| | | | | FTFGDYDMHWVRQAPGKGLEWVSAI |
| | | | | SSSGSNRDYADSVEGRFTISRDNAKNS |
| | | | | LYLQMNSLRAEDTAVYYCARDAWFG |
| | | | | DAFDIWGQGTMVTVSS |
| | | CDR H1 | 167 | EFTFGDYD |
| | | CDR H2 | 168 | ISSSGSNR |
| | | CDR H3 | 169 | ARDAWFGDAFDI |
| | VL | All | 170 | DIQMTQSPSSLSASVGDRVTITCRASE |
| | | | | SVSSWLAWYQQKPGKAPKLLIYGAST |
| | | | | LEGGVPSRFSGSGSGTDFTLTISSLQPE |
| | | | | DVATYYCQQSYSFPDFGGGTKVEIK |
| | | CDR L1 | 171 | ESVSSW |
| | | CDR L2 | | GAS |
| | | CDR L3 | 172 | QQSYSFPD |
| 2738_Lib1 | VH | All | 173 | QVQLVQSGAEVKKPGASVKVSCKVS |
| | | | | GYTLTDLPIHWVRQAPGKGLEWMGG |
| | | | | INPFLGTTIYAQKFQGRVTMTEDTSTD |
| | | | | TAYMELSSLKSEDTAVYYCARGVDM |
| | | | | LATARDYYYMAIWGKGTTVTVSS |
| | | CDR H1 | 174 | GYTLTDLP |
| | | CDR H2 | 175 | INPFLGTT |
| | | CDR H3 | 176 | ARGVDMLATARDYYYMAI |
| | VL | All | 177 | DIQMTQSPSSVSASVGDRVTITCRASQ |
| | | | | SIGNDLAWYQQKPGKAPKLLIYGASM |
| | | | | RATGVPSRFSGSGSGTDFTLTISSLQPE |
| | | | | DFANYYCQQYASDPLTFGGGTKVEIK |
| | | CDR L1 | 178 | QSIGND |
| | | CDR L2 | | GAS |
| | | CDR L3 | 179 | QQYASDPLT |
| Lib4_P2_A1 | VH | All | 180 | QVQLQESGPGLVKPSQTLSLTCTVSG |
| | | | | ASFGDNYWSWIRQPPGKGLEWIGYID |
| | | | | TSGSSDYNPSLKSRVTMSVDTSKNQF |
| | | | | SLKVNSVTAADTAVYYCARVSWGYY |
| | | | | AMDVWGQGTTVTVSS |
| | | CDR H1 | 181 | GASFGDNY |
| | | CDR H2 | 182 | IDTSGSS |
| | | CDR H3 | 183 | ARVSWGYYAMDV |
| | VL | All | 184 | EIVMTQSPATLSLSPGERATLSCRASQ |
| | | | | GISGRLAWYQQKPGQAPRLLIYDVST |
| | | | | RATGIPARFSGSGSGTDFTLTISSLEPE |
| | | | | DFAVYYCQQYDTSAPLTFGGGTKVEI |
| | | | | K |
| | | CDR L1 | 185 | QGISGR |
| | | CDR L2 | | DVS |
| | | CDR L3 | 186 | QQYDTSAPLT |
| 189_Lib4 | VH | All | 187 | QVQLQESGPGLVKPSQTLSLTCTVSG |
| | | | | VTFSSYGWSWIRQPPGKGLEWIGYVD |
| | | | | TSGSTDYNPSLKSRVTMSVDTSKNQF |
| | | | | SLKVNSVTAADTAVYYCARTTYYYD |
| | | | | SSGFARGWFDPWGQGTLVTVSS |
| | | CDR H1 | 188 | GVTFSSYG |
| | | CDR H2 | 189 | VDTSGST |
| | | CDR H3 | 190 | ARTTYYYDSSGFARGWFDP |
| | VL | All | 191 | EIVMTQSPATLSLSPGERATLSCRASQ |
| | | | | SVPSSHLAWYQQKPGQAPRLLIYDAT |
| | | | | NRAAGIPARFSGSGSGTDFTLTISSLEP |
| | | | | EDFAVYYCQQYDNWPSPSFGGGTKV |
| | | | | EIK |
| | | CDR L1 | 192 | QSVPSSH |
| | | CDR L2 | | DAT |
| | | CDR L3 | 193 | QQYDNWPSPS |
| Lib1_P1_A1 | VH | All | 194 | QVQLVQSGAEVKKPGASVKVSCKVS |
| | | | | GDTLSNYAIHWVRQAPGKGLEWMGG |
| | | | | INPNAGTTIYAQKFQGRVTMTEDTST |
| | | | | DTAYMELSSLKSEDTAVYYCARDSSS |
| | | | | WSQFGYYYYYMDVWGKGTTVTVSS |
| | | CDR H1 | 195 | GDTLSNYA |
| | | CDR H2 | 196 | INPNAGTT |
| | | CDR H3 | 197 | ARDSSSWSQFGYYYYYMDV |
| | VL | All | 198 | DIQMTQSPSSVSASVGDRVTITCRASQ |
| | | | | SIGDFLAWYQQKPGKAPKLLIYDASH |
| | | | | LQTGVPSRFSGSGSGTDFTLTISSLQPE |
| | | | | DFANYYCQQTSTIPPTFGGGTKVEIK |
| | | CDR L1 | 199 | QSIGDF |
| | | CDR L2 | | DAS |
| | | CDR L3 | 200 | QQTSTIPPT |
| Lib6_P3_B9 | VH | All | 201 | EVQLVQSGAEVKKPGASVKVSCKAS |
| | | | | GDTFSSHVISWVRQAPGQGLEWMGGI |
| | | | | NPRTGTTIYAQKLQGRGTMTTDPSTS |
| | | | | TAYMELRSLRSDDTAVYYCARTRPW |
| | | | | AGLDVWGQGTTVTVSS |
| | | CDR H1 | 202 | GDTFSSHV |
| | | CDR H2 | 203 | INPRTGTT |
| | | CDR H3 | 204 | ARTRPWAGLDV |
| | VL | All | 205 | ESALTQPASVSGSPGQSITISCTGTNIE |
| | | | | YKGVSWYQQHPGKAPKLMIYGVDKR |
| | | | | PSGVSNRFSGSKSGNTASLTISGLQAE |
| | | | | DEADYYCQAWGTSTAIFGGGTKLTVL |
| | | CDR L1 | 206 | NIEYKG |
| | | CDR L2 | | GVD |
| | | CDR L3 | 207 | QAWGTSTAI |
| Lib6_P3_A1 | VH | All | 208 | QVQLVQSGAEVKKPGASVKVSCKVS |
| | | | | GDTLSNYAIHWVRQAPGKGLEWMGG |
| | | | | INPNAGTTIYAQKFQGRVTMTEDTST |
| | | | | DTAYMELSSLKSEDTAVYYCARDSSS |
| | | | | WCPVGYYYYYMDVWGKGTTVTVSS |
| | | CDR H1 | 209 | GDTLSNYA |
| | | CDR H2 | 210 | INPNAGTT |
| | | CDR H3 | 211 | ARDSSSWCPVGYYYYYMDV |
| | VL | All | 212 | DIQMTQSPSSVSASVGDRVTITCRASQ |
| | | | | SIGDFLAWYQQKPGKAPKLLIYDASH |
| | | | | LQTGVPSRFSGSGSGTDFTLTISSLQPE |
| | | | | DFANYYCQQTSTIPPTFGGGTKVEIK |
| | | CDR L1 | 213 | QSIGDF |
| | | CDR L2 | | DAS |
| | | CDR L3 | 214 | QQTSTIPPT |
| Lib4_P2_A3 | VH | All | 215 | QVQLQESGPGLVKPSQTLSLTCTVSG |
| | | | | VSLSTSDYSWSWIRQPPGKGLEWIGYI |
| | | | | SPPGRSDYNPSLKSRVTMSVDTSKNQ |
| | | | | FSLKVNSVTAADTAVYYCARHSSSG |
| | | | | WYSTYDFDSWGQGTMVTVSS |
| | | CDR H1 | 216 | GVSLSTSDYS |
| | | CDR H2 | 217 | ISPPGRS |
| | | CDR H3 | 218 | ARHSSSGWYSTYDFDS |
| | VL | All | 219 | EIVMTQSPATLSLSPGERATLSCRASQ |
| | | | | NIHSNLAWYQQKPGQAPRLLIYGATT |
| | | | | RATGIPARFSGSGSGTDFTLTISSLEPE |
| | | | | DFAVYYCQQSYTTPLTFGGGTKVEIK |
| | | CDR L1 | 220 | QNIHSN |
| | | CDR L2 | | GAT |
| | | CDR L3 | 221 | QQSYTTPLT |
| 487_Lib1 | VH | All | 222 | QVQLVQSGAEVKKPGASVKVSCKVS |
| | | | | GYTFTSHIIHWVRQAPGKGLEWMGG |
| | | | | MNPYSNKAIYAQKFQGRVTMTEDTST |
| | | | | DTAYMELSSLKSEDTAVYYCARDAS |
| | | | | DYGDYGYYYYYMDVWGKGTTVTVS |
| | | | | S |
| | | CDR H1 | 223 | GYTFTSHI |
| | | CDR H2 | 224 | MNPYSNKA |
| | | CDR H3 | 225 | ARDASDYGDYGYYYYYMDV |
| | VL | All | 226 | DIQMTQSPSSVSASVGDRVTITCRASQ |
| | | | | SIGDFLAWYQQKPGKAPKLLIYDASH |
| | | | | LQTGVPSRFSGSGSGTDFTLTISSLQPE |
| | | | | DFANYYCQQTSTIPPTFGGGTKVEIK |
| | | CDR L1 | 227 | QSIGDF |
| | | CDR L2 | | DAS |
| | | CDR L3 | 228 | QQTSTIPPT |
| Lib6_P3_E10 | VH | All | 229 | QVQLVQSGAEVKKPGASVKVSCKVS |
| | | | | GDTLSNYAIHWVRQAPGKGLEWMGG |
| | | | | INPNAGTTIYAQKFQGRVTMTEDTST |
| | | | | DTAYMELSSLKSEDTAVYYCARDSSS |
| | | | | WCPVGYYYYYMDVWGKGTTVTVSS |
| | | CDR H1 | 230 | GDTLSNYA |
| | | CDR H2 | 231 | INPNAGTT |
| | | CDR H3 | 232 | ARDSSSWCPVGYYYYYMDV |
| | VL | All | 233 | DIQMTQSPSSVSASVGDRVTITCRASQ |
| | | | | SIGDFLAWYQQKPGKAPKLLIYDASH |
| | | | | LQTGVPSRFSGSGSGTDFTLTISSLQPE |
| | | | | DFANYYCQQTSTIPPTFGGGTKVEIK |
| | | CDR L1 | 234 | QSIGDF |
| | | CDR L2 | | DAS |
| | | CDR L3 | 235 | QQTSTIPPT |
| 256_Lib8 | VH | All | 236 | EVQLVESGGGLVQPGRSLRLSCAASG |
| | | | | FTVSSNAMHWVRQAPGKGLEWVSAI |
| | | | | GTFADTDYADSVEGRFTISRDNAKNS |
| | | | | LYLQMNSLRAEDTAVYYCAREGAAA |
| | | | | GTNFDYWGQGTLVTVSS |
| | | CDR H1 | 237 | GFTVSSNA |
| | | CDR H2 | 238 | IGTFADT |
| | | CDR H3 | 239 | AREGAAAGTNFDY |
| | VL | All | 240 | DIQMTQSPSSLSASVGDRVTITCRASQ |
| | | | | GIRSYLAWYQQKPGKAPKLLIYGAST |
| | | | | LEGGVPSRFSGSGSGTDFTLTISSLQPE |
| | | | | DVATYYCQQSYSTPPKFGGGTKVEIK |
| | | CDR L1 | 24 | QGIRSY |
| | | CDR L2 | | GAS |
| | | CDR L3 | 242 | QQSYSTPPK |
| 4035_Lib1 | VH | All | 243 | QVQLVQSGAEVKKPGASVKVSCKVS |
| | | | | GYAFPSYGIHWVRQAPGKGLEWMGG |
| | | | | ISAFTGDTIYAQKFQGRVTMTEDTSTD |
| | | | | TAYMELSSLKSEDTAVYYCASGPTNV |
| | | | | LTGLMESAEYFQHWGQGTLVTVSS |
| | | CDR H1 | 244 | GYAFPSYG |
| | | CDR H2 | 245 | ISAFTGDT |
| | | CDR H3 | 246 | ASGPTNVLTGLMESAEYFQH |
| | VL | All | 247 | DIQMTQSPSSVSASVGDRVTITCRASQ |
| | | | | GISRDLAWYQQKPGKAPKLLIYGASG |
| | | | | RATGVPSRFSGSGSGTDFTLTISSLQPE |
| | | | | DFANYYCQQSYITPTTFGGGTKVEIK |
| | | CDR L1 | 248 | QGISRD |
| | | CDR L2 | | GAS |
| | | CDR L3 | 249 | QQSYITPTT |
| 96_Lib4 | VH | All | 250 | QVQLQESGPGLVKPSQTLSLTCTVSGE |
| | | | | SFSTSYWSWIRQPPGKGLEWIGYITHS |
| | | | | GYTDYNPSLKSRVTMSVDTSKNQFSL |
| | | | | KVNSVTAADTAVYYCARGGRFPFWY |
| | | | | FDLWGRGSLVTVSS |
| | | CDR H1 | 251 | GESFSTSY |
| | | CDR H2 | 252 | ITHSGYT |
| | | CDR H3 | 253 | ARGGRFPFWYFDL |
| | VL | All | 254 | EIVMTQSPATLSLSPGERATLSCRASR |
| | | | | SIGSNLAWYQQKPGQAPRLLIYGATT |
| | | | | RATGIPARFSGSGSGTDFTLTISSLEPE |
| | | | | DFAVYYCQQYKNDATFGGGTKVEIK |
| | | CDR L1 | 255 | RSIGSN |
| | | CDR L2 | | GAT |
| | | CDR L3 | 256 | QQYKNDAT |
| Lib1_P1_A2 | VH | All | 257 | QVQLVQSGAEVKKPGASVKVSCKVS |
| | | | | EGTLSRHYINWVRQAPGKGLEWMGG |
| | | | | INAKSGATIYAQKFQGRVTMTEDTST |
| | | | | DTAYMELSSLKSEDTAVYYCAKDTG |
| | | | | DDILTDYSDAFDIWGQGTMVTVSS |
| | | CDR H1 | 258 | EGTLSRHY |
| | | CDR H2 | 259 | INAKSGAT |
| | | CDR H3 | 260 | AKDTGDDILTDYSDAFDI |
| | VL | All | 261 | DIQMTQSPSSVSASVGDRVTITCRASQ |
| | | | | NIGTSLAWYQQKPGKAPKLLIYGASN |
| | | | | LESGVPSRFSGSGSGTDFTLTISSLQPE |
| | | | | DFANYYCLQYNDWPPLYSFGGGTKV |
| | | | | EIK |
| | | CDR L1 | 262 | QNIGTS |
| | | CDR L2 | | GAS |
| | | CDR L3 | 263 | LQYNDWPPLYS |
| 2301_Lib4 | VH | All | 264 | QVQLQESGPGLVKPSQTLSLTCTVSG |
| | | | | ASISTSDHSWSWIRQPPGKGLEWIGYI |
| | | | | RDTGSTDYNPSLKSRVTMSVDTSKNQ |
| | | | | FSLKVNSVTAADTAVYYCAISSTERW |
| | | | | KQSFGVDTEYFQHWGQGTLVTVSS |
| | | CDR H1 | 265 | GASISTSDHS |
| | | CDR H2 | 266 | IRDTGST |
| | | CDR H3 | 267 | AISSTERWKQSFGVDTEYFQH |
| | VL | All | 268 | EIVMTQSPATLSLSPGERATLSCRASR |
| | | | | YVSTNLAWYQQKPGQAPRLLIYDAV |
| | | | | TRAPGIPARFSGSGSGTDFTLTISSLEP |
| | | | | EDFAVYYCQQYHSLPLIFGGGTKVEIK |
| | | CDR L1 | 269 | RYVSTN |
| | | CDR L2 | | DAV |
| | | CDR L3 | 270 | QQYHSLPLI |
| Lib4_P2_B1 | VH | All | 27 | QVQLVQSGAEVKKPGASVKVSCKVS |
| | | | | GDTLSNYAIHWVRQAPGKGLEWMGG |
| | | | | INPNAGTTIYAQKFQGRVTMTEDTST |
| | | | | DTAYMELSSLKSEDTAVYYCARDSSS |
| | | | | WCPVGYYYYYMDVWGKGTTVTVSS |
| | | CDR H1 | 272 | GDTLSNYA |
| | | CDR H2 | 273 | INPNAGTT |
| | | CDR H3 | 274 | ARDSSSWCPVGYYYYYMDV |
| | VL | All | 275 | DIQMTQSPSSVSASVGDRVTITCRASQ |
| | | | | SIGDFLAWYQQKPGKAPKLLIYDASH |
| | | | | LQTGVPSRFSGSGSGTDFTLTISSLQPE |
| | | | | DFANYYCQQTSTIPPTFGGGTKVEIK |
| | | CDR L1 | 276 | QSIGDF |
| | | CDR L2 | | DAS |
| | | CDR L3 | 277 | QQTSTIPPT |
| Lib4_P2_A2 | VH | All | 278 | QVQLVQSGAEVKKPGASVKVSCKVS |
| | | | | GDTLSNYAIHWVRQAPGKGLEWMGG |
| | | | | INPNAGTTIYAQKFQGRVTMTEDTST |
| | | | | DTAYMELSSLKSEDTAVYYCARDSSS |
| | | | | WCPVGYYYYYMDVWGKGTTVTVSS |
| | | CDR H1 | 279 | GDTLSNYA |
| | | CDR H2 | 280 | INPNAGTT |
| | | CDR H3 | 281 | ARDSSSWCPVGYYYYYMDV |
| | VI | All | 282 | DIQMTQSPSSVSASVGDRVTITCRASQ |
| | | | | SIGDFLAWYQQKPGKAPKLLIYDASH |
| | | | | LQTGVPSRFSGSGSGTDFTLTISSLQPE |
| | | | | DFANYYCQQTSTIPPTFGGGTKVEIK |
| | | CDR L1 | 283 | QSIGDF |
| | | CDR L2 | | DAS |
| | | CDR L3 | 284 | QQTSTIPPT |
| 16_Lib8 | VH | All | 285 | EVQLVESGGGLVQPGRSLRLSCAASG |
| | | | | FRFDEYDMHWVRQAPGKGLEWVSAI |
| | | | | SSESASIDYADSVEGRFTISRDNAKNS |
| | | | | LYLQMNSLRAEDTAVYYCARDGAYG |
| | | | | DYFDYWGQGTMVTVSS |
| | | CDR H1 | 286 | GFRFDEYD |
| | | CDR H2 | 287 | ISSESASI |
| | | CDR H3 | 288 | ARDGAYGDYFDY |
| | VL | All | 289 | DIQMTQSPSSLSASVGDRVTITCRASQ |
| | | | | DIKSWLAWYQQKPGKAPKLLIYGAST |
| | | | | LQSGVPSRFSGSGSGTDFTLTISSLQPE |
| | | | | DVATYYCQQSYSAPYTFGGGTKVEIK |
| | | CDR L1 | 290 | QDIKSW |
| | | CDR L2 | | GAS |
| | | CDR L3 | 29 | QQSYSAPYT |
| 11485_Lib1 | VH | All | 292 | QVQLVQSGAEVKKPGASVKVSCKVS |
| | | | | GAPSAAIIHWVRQAPGKGLEWMGGF |
| | | | | TQIFGTAIYAQKFQGRVTMTEDTSTDT |
| | | | | AYMELSSLKSEDTAVYYCAREGIGGH |
| | | | | DAFDIWGQGTMVTVSS |
| | | CDR H1 | 293 | GAPSAAI |
| | | CDR H2 | 294 | FTQIFGTA |
| | | CDR H3 | 295 | AREGIGGHDAFDI |
| | VL | All | 296 | DIQMTQSPSSVSASVGDRVTITCRASQ |
| | | | | DISKNLAWYQQKPGKAPKLLIYKASK |
| | | | | LESGVPSRFSGSGSGTDFTLTISSLQPE |
| | | | | DFANYYCLHDYTYPPTFGGGTKVEIK |
| | | CDR L1 | 297 | QDISKN |
| | | CDR L2 | | KAS |
| | | CDR L3 | 298 | LHDYTYPPT |
| 4956_Lib1 | VH | All | 299 | QVQLVQSGAEVKKPGASVKVSCKVS |
| | | | | GYTLTSNYIHWVRQAPGKGLEWMGG |
| | | | | FNPSSGSTIYAQKFQGRVTMTEDTSTD |
| | | | | TAYMELSSLKSEDTAVYYCARTLYDF |
| | | | | WSGYLDAFDIWGQGTMVTVSS |
| | | CDR H1 | 300 | GYTLTSNY |
| | | CDR H2 | 301 | FNPSSGST |
| | | CDR H3 | 302 | ARTLYDFWSGYLDAFDI |
| | VL | All | 303 | DIQMTQSPSSVSASVGDRVTITCRASQ |
| | | | | NIRSSLAWYQQKPGKAPKLLIYAASN |
| | | | | LEPGVPSRFSGSGSGTDFTLTISSLQPE |
| | | | | DFANYYCQQYEDTPLTFGGGTKVEIK |
| | | CDR L1 | 304 | QNIRSS |
| | | CDR L2 | | AAS |
| | | CDR L3 | 305 | QQYEDTPLT |
| 399_Lib1 | VH | All | 306 | QVQLVQSGAEVKKPGASVKVSCKVS |
| | | | | GITFTSDYIHWVRQAPGKGLEWMGG |
| | | | | MNPYSNKAIYAQKFQGRVTMTEDTST |
| | | | | DTAYMELSSLKSEDTAVYYCATGGLR |
| | | | | IAARTSYFDYWGQGTLVTVSS |
| | | CDR H1 | 307 | GITFTSDY |
| | | CDR H2 | 308 | MNPYSNKA |
| | | CDR H3 | 309 | ATGGLRIAARTSYFDY |
| | VL | All | 310 | DIQMTQSPSSVSASVGDRVTITCRASM |
| | | | | DIAGSLAWYQQKPGKAPKLLIYDTSF |
| | | | | RATGVPSRFSGSGSGTDFTLTISSLQPE |
| | | | | DFANYYCQQYDNPPRLTFGGGTKVEI |
| | | | | K |
| | | CDR L1 | 311 | MDIAGS |
| | | CDR L2 | | DTS |
| | | CDR L3 | 312 | QQYDNPPRLT |
| Lib4_P2_A10 | VH | All | 313 | QVQLQESGPGLVKPSQTLSLTCTVSG |
| | | | | ASLGHQYWSWIRQPPGKGLEWIGYID |
| | | | | TSGSTDYNPSLKSRVTMSVDTSKNQF |
| | | | | SLKVNSVTAADTAVYYCARVANYYY |
| | | | | YMDVWGKGTTVTVSS |
| | | CDR H1 | 314 | GASLGHQY |
| | | CDR H2 | 315 | IDTSGST |
| | | CDR H3 | 316 | ARVANYYYYMDV |
| | VL | All | 317 | EIVMTQSPATLSLSPGERATLSCRASQ |
| | | | | SVSNRLAWYQQKPGQAPRLLIYDASN |
| | | | | RATGIPARFSGSGSGTDFTLTISSLEPE |
| | | | | DFAVYYCQQYFITPSTFGGGTKVEIK |
| | | CDR L1 | 318 | QSVSNR |
| | | CDR L2 | | DAS |
| | | CDR L3 | 319 | QQYFITPST |
| Lib6_P3_A8 | VH | All | 320 | EVQLVQSGAEVKKPGASVKVSCKAS |
| | | | | GYRFTNYDISWVRQAPGQGLEWMGG |
| | | | | INPSSGITIYAQKLQGRGTMTTDPSTST |
| | | | | AYMELRSLRSDDTAVYYCARGAAYG |
| | | | | DYLDYWGQGTLVTVSS |
| | | CDR H1 | 321 | GYRFTNYD |
| | | CDR H2 | 322 | INPSSGIT |
| | | CDR H3 | 323 | ARGAAYGDYLDY |
| | VL | All | 324 | ESALTQPASVSGSPGQSITISCTGTNLG |
| | | | | EKYVSWYQQHPGKAPKLMIYSTSQRP |
| | | | | SGVSNRFSGSKSGNTASLTITGLQAED |
| | | | | EADYYCSSYTASSTPGLVFGGGTKLT |
| | | | | VL |
| | | CDR L1 | 325 | NLGEKY |
| | | CDR L2 | | STS |
| | | CDR L3 | 326 | SSYTASSTPGLV |
| 965_Lib4 | VH | All | 327 | QVQLQESGPGLVKPSQTLSLTCTVSG |
| | | | | ASFGDNYWSWIRQPPGKGLEWIGYID |
| | | | | TSGSSDYNPSLKSRVTMSVDTSKNQF |
| | | | | SLKVNSVTAADTAVYYCARDALGYY |
| | | | | YMDVWGKGTTVTVSS |
| | | CDR H1 | 328 | GASFGDNY |
| | | CDR H2 | 329 | IDTSGSS |
| | | CDR H3 | 330 | ARDALGYYYMDV |
| | VL | All | 331 | EIVMTQSPATLSLSPGERATLSCRASQ |
| | | | | GISGRLAWYQQKPGQAPRLLIYDASA |
| | | | | RASGIPARFSGSGSGTDFTLTISSLEPE |
| | | | | DFAVYYCQQYGTSPYTFGGGTKVEIK |
| | | CDR L1 | 332 | QGISGR |
| | | CDR L2 | | DAS |
| | | CDR L3 | 333 | QQYGTSPYT |
| 27_Lib4 | VH | All | 334 | QVQLQESGPGLVKPSQTLSLTCTVSG |
| | | | | ASFGDHYWSWIRQPPGKGLEWIGYID |
| | | | | ATGSIDYNPSLKSRVTMSVDTSKNQFS |
| | | | | LKVNSVTAADTAVYYCARVASYYYY |
| | | | | MDVWGKGTTVTVSS |
| | | CDR H1 | 335 | GASFGDHY |
| | | CDR H2 | 336 | IDATGSI |
| | | CDR H3 | 337 | ARVASYYYYMDV |
| | VL | All | 338 | EIVMTQSPATLSLSPGERATLSCRASQ |
| | | | | SITGSYLAWYQQKPGQAPRLLIYGAS |
| | | | | DRAPGIPARFSGSGSGTDFTLTISSLEP |
| | | | | EDFAVYYCQQYGSFPYTFGGGTKVEI |
| | | | | K |
| | | CDR L1 | 339 | QSITGSY |
| | | CDR L2 | | GAS |
| | | CDR L3 | 340 | QQYGSFPYT |
| 15244_Lib8 | VH | All | 341 | EVQLVESGGGLVQPGRSLRLSCAASG |
| | | | | FTFADYDMHWVRQAPGKGLEWVSA |
| | | | | TSSHGVTTDYADSVEGRFTISRDNAK |
| | | | | NSLYLQMNSLRAEDTAVYYCARGAW |
| | | | | SGIAARQYIDYWGQGTLVTVSS |
| | | CDR H1 | 342 | GFTFADYD |
| | | CDR H2 | 343 | TSSHGVTT |
| | | CDR H3 | 344 | ARGAWSGIAARQYIDY |
| | VL | All | 345 | DIQMTQSPSSLSASVGDRVTITCRASQ |
| | | | | TIGYWLAWYQQKPGKAPKLLIYDAS |
| | | | | VPESGVPSRFSGSGSGTDFTLTISSLQP |
| | | | | EDVATYYCQQSYSYPLTFGGGTKVEI |
| | | | | K |
| | | CDR L1 | 346 | QTIGYW |
| | | CDR L2 | | DAS |
| | | CDR L3 | 347 | QQSYSYPLT |
| Lib4_P2_All | VH | All | 348 | QVQLQESGPGLVKPSQTLSLTCTVSG |
| | | | | VTFSSYGWSWIRQPPGKGLEWIGYVD |
| | | | | TSGSTDYNPSLKSRVTMSVDTSKNQF |
| | | | | SLKVNSVTAADTAVYYCARTTYYYD |
| | | | | SSGFARGWFDPWGQGTLVTVSS |
| | | CDR H1 | 349 | GVTFSSYG |
| | | CDR H2 | 350 | VDTSGST |
| | | CDR H3 | 351 | ARTTYYYDSSGFARGWFDP |
| | VL | All | 352 | EIVMTQSPATLSLSPGERATLSCRASR |
| | | | | SISTNLAWYQRKPGQAPRLLIYDTSNR |
| | | | | APGIPARFSGSGSGTDFTLTISSLEPED |
| | | | | FAVYYCLQYDVSPYTFGGGTKVEIK |
| | | CDR L1 | 353 | RSISTN |
| | | CDR L2 | | DTS |
| | | CDR L3 | 354 | LQYDVSPYT |
| Lib8_P4_C10 | VH | All | 355 | EVQLVESGGGLVQPGRSLRLSCAASG |
| | | | | FNFHNYDMHWVRQAPGKGLEWVSAI |
| | | | | SGSSDTMDYADSVEGRFTISRDNAKN |
| | | | | SLYLQMNSLRAEDTAVYYCVRASWY |
| | | | | GTFFDYWGQGTLVTVSS |
| | | CDR H1 | 356 | GFNFHNYD |
| | | CDR H2 | 357 | ISGSSDTM |
| | | CDR H3 | 358 | VRASWYGTFFDY |
| | VL | All | 359 | DIQMTQSPSSLSASVGDRVTITCRASQ |
| | | | | DISGWLAWYQQKPGKAPKLLIYAASS |
| | | | | LQDGVPSRFSGSGSGTDFTLTISSLQPE |
| | | | | DVATYYCQQSYRIPSTFGGGTKVEIK |
| | | CDR L1 | 360 | QDISGW |
| | | CDR L2 | | AAS |
| | | CDR L3 | 361 | QQSYRIPST |
| 8226_Lib8 | VH | All | 362 | EVQLVESGGGLVQPGRSLRLSCAASG |
| | | | | FNFHNYDMHWVRQAPGKGLEWVSAI |
| | | | | SGGAVSTDYADSVEGRFTISRDNAKN |
| | | | | SLYLQMNSLRAEDTAVYYCARSSWY |
| | | | | GGQIDFWGQGTLVTVSS |
| | | CDR H1 | 363 | GFNFHNYD |
| | | CDR H2 | 364 | ISGGAVST |
| | | CDR H3 | 365 | ARSSWYGGQIDF |
| | VL | All | 366 | DIQMTQSPSSLSASVGDRVTITCRASQ |
| | | | | DISGWLAWYQQKPGKAPKLLIYAASS |
| | | | | LQDGVPSRFSGSGSGTDFTLTISSLQPE |
| | | | | DVATYYCQQSYRIPSTFGGGTKVEIK |
| | | CDR L1 | 367 | QDISGW |
| | | CDR L2 | | AAS |
| | | CDR L3 | 368 | QQSYRIPST |
| 69_Lib8 | VH | All | 369 | EVQLVESGGGLVQPGRSLRLSCAASA |
| | | | | FTFSNYDMHWVRQAPGKGLEWVSAI |
| | | | | SGSGDTSDYADSVEGRFTISRDNAKN |
| | | | | SLYLQMNSLRAEDTAVYYCVRASWY |
| | | | | GTFFDYWGQGTLVTVSS |
| | | CDR H1 | 370 | AFTFSNYD |
| | | CDR H2 | 371 | ISGSGDTS |
| | | CDR H3 | 372 | VRASWYGTFFDY |
| | VL | All | 373 | DIQMTQSPSSLSASVGDRVTITCRASQ |
| | | | | SVSGWLAWYQQKPGKAPKLLIYGTST |
| | | | | LQSGVPSRFSGSGSGTDFTLTISSLQPE |
| | | | | DVATYYCQQSYSAPYTFGGGTKVEIK |
| | | CDR L1 | 374 | QSVSGW |
| | | CDR L2 | | GTS |
| | | CDR L3 | 375 | QQSYSAPYT |
| 76_Lib4 | VH | All | 376 | QVQLQESGPGLVKPSQTLSLTCTVSG |
| | | | | VSISNHYWSWIRQPPGKGLEWIGYIDE |
| | | | | SGSTDYNPSLKSRVTMSVDTSKNQFS |
| | | | | LKVNSVTAADTAVYYCARVSWGYY |
| | | | | AMDVWGQGTTVTVSS |
| | | CDR H1 | 377 | GVSISNHY |
| | | CDR H2 | 378 | IDESGST |
| | | CDR H3 | 379 | ARVSWGYYAMDV |
| | VL | All | 380 | EIVMTQSPATLSLSPGERATLSCRASQ |
| | | | | GVSGHLAWYQQKPGQAPRLLIYETSK |
| | | | | RATGIPARFSGSGSGTDFTLTISSLEPE |
| | | | | DFAVYYCQQYNDYPLGFGGGTKVEI |
| | | | | K |
| | | CDR L1 | 381 | QGVSGH |
| | | CDR L2 | | ETS |
| | | CDR L3 | 382 | QQYNDYPLG |
| 4675_Lib4 | VH | All | 383 | QVQLQESGPGLVKPSQTLSLTCTVSGF |
| | | | | SLTSHPEGVNWIRQPPGKGLEWIGYIA |
| | | | | HSGTTDYNPSLKSRVTMSVDTSKNQF |
| | | | | SLKVNSVTAADTAVYYCARAYGDSD |
| | | | | AFDIWGQGTMVTVSS |
| | | CDR H1 | 384 | GFSLTSHPEG |
| | | CDR H2 | 385 | IAHSGTT |
| | | CDR H3 | 386 | ARAYGDSDAFDI |
| | VL | All | 387 | EIVMTQSPATLSLSPGERATLSCRASQ |
| | | | | SIGRNLAWYQQKPGQAPRLLIYGASK |
| | | | | RVTGIPARFSGSGSGTDFTLTISSLEPE |
| | | | | DFAVYYCQQYSTWPTFGGGTKVEIK |
| | | CDR L1 | 388 | QSIGRN |
| | | CDR L2 | | GAS |
| | | CDR L3 | 389 | QQYSTWPT |
| 4029_Lib1 | VH | All | 390 | QVQLVQSGAEVKKPGASVKVSCKVS |
| | | | | GYPFTSSGIHWVRQAPGKGLEWMGG |
| | | | | VIPLFGTANYAQKFQGRVTMTEDTST |
| | | | | DTAYMELSSLKSEDTAVYYCARIRVS |
| | | | | SGWYRDYGMDVWGQGTTVTVSS |
| | | CDR H1 | 39 | GYPFTSSG |
| | | CDR H2 | 392 | VIPLFGTA |
| | | CDR H3 | 393 | ARIRVSSGWYRDYGMDV |
| | VL | All | 394 | DIQMTQSPSSVSASVGDRVTITCRASQ |
| | | | | SIGDFLAWYQQKPGKAPKLLIYDASH |
| | | | | LQTGVPSRFSGSGSGTDFTLTISSLQPE |
| | | | | DFANYYCQQTSTIPPTFGGGTKVEIK |
| | | CDR L1 | 395 | QSIGDF |
| | | CDR L2 | | DAS |
| | | CDR L3 | 396 | QQTSTIPPT |
| 5662_Lib8 | VH | All | 397 | EVQLVESGGGLVQPGRSLRLSCAASG |
| | | | | FNFHNYDMHWVRQAPGKGLEWVSAI |
| | | | | DISGQSTDYADSVEGRFTISRDNAKNS |
| | | | | LYLQMNSLRAEDTAVYYCAREGWYG |
| | | | | GVFDYWGQGTLVTVSS |
| | | CDR H1 | 398 | GFNFHNYD |
| | | CDR H2 | 399 | IDISGQST |
| | | CDR H3 | 400 | AREGWYGGVFDY |
| | VL | All | 401 | DIQMTQSPSSLSASVGDRVTITCRASQ |
| | | | | DIANWLAWYQQKPGKAPKLLIYGAS |
| | | | | ESGVGVPSRFSGSGSGTDFTLTISSLQP |
| | | | | EDVATYYCQQSLTMKTFGGGTKVEIK |
| | | CDR L1 | 402 | QDIANW |
| | | CDR L2 | | GAS |
| | | CDR L3 | 403 | QQSLTMKT |
| Lib6_P3_A4 | VH | All | 404 | QVQLVQSGAEVKKPGASVKVSCKVS |
| | | | | GDTLSNYAIHWVRQAPGKGLEWMGG |
| | | | | INPNAGTTIYAQKFQGRVTMTEDTST |
| | | | | DTAYMELSSLKSEDTAVYYCARDSSS |
| | | | | WCPVGYYYYYMDVWGKGTTVTVSS |
| | | CDR H1 | 405 | GDTLSNYA |
| | | CDR H2 | 406 | INPNAGTT |
| | | CDR H3 | 407 | ARDSSSWCPVGYYYYYMDV |
| | VL | All | 408 | DIQMTQSPSSVSASVGDRVTITCRASQ |
| | | | | SIGDFLAWYQQKPGKAPKLLIYDASH |
| | | | | LQTGVPSRFSGSGSGTDFTLTISSLQPE |
| | | | | DFANYYCQQTSTIPPTFGGGTKVEIK |
| | | CDR L1 | 409 | QSIGDF |
| | | CDR L2 | | DAS |
| | | CDR L3 | 410 | QQTSTIPPT |
| Lib1_P1_F10 | VH | All | 411 | QVQLVQSGAEVKKPGASVKVSCKVS |
| | | | | GYTFSSSGIHWVRQAPGKGLEWMGGI |
| | | | | FPNFGTAIYAQKFQGRVTMTEDTSTD |
| | | | | TAYMELSSLKSEDTAVYYCARESDFW |
| | | | | SGSFTNWGQGTLVTVSS |
| | | CDR H1 | 412 | GYTFSSSG |
| | | CDR H2 | 413 | IFPNFGTA |
| | | CDR H3 | 414 | ARESDFWSGSFTN |
| | VL | All | 415 | DIQMTQSPSSVSASVGDRVTITCRASR |
| | | | | SIGNWLAWYQQKPGKAPKLLIYDAFN |
| | | | | RASGVPSRFSGSGSGTDFTLTISSLQPE |
| | | | | DFANYYCQQSYSTLPRYSFGGGTKVE |
| | | | | IK |
| | | CDR L1 | 416 | RSIGNW |
| | | CDR L2 | | DAF |
| | | CDR L3 | 417 | QQSYSTLPRYS |
| Lib4_P2_D1 | VH | All | 418 | QVQLVQSGAEVKKPGASVKVSCKVS |
| | | | | GDTLSNYAIHWVRQAPGKGLEWMGG |
| | | | | INPNAGTTIYAQKFQGRVTMTEDTST |
| | | | | DTAYMELSSLKSEDTAVYYCARDSSS |
| | | | | WCPVGYYYYYMDVWGKGTTVTVSS |
| | | CDR H1 | 419 | GDTLSNYA |
| | | CDR H2 | 420 | INPNAGTT |
| | | CDR H3 | 421 | ARDSSSWCPVGYYYYYMDV |
| | VL | All | 422 | DIQMTQSPSSVSASVGDRVTITCRASQ |
| | | | | SIGDFLAWYQQKPGKAPKLLIYDASH |
| | | | | LQTGVPSRFSGSGSGTDFTLTISSLQPE |
| | | | | DFANYYCQQTSTIPPTFGGGTKVEIK |
| | | CDR L1 | 423 | QSIGDF |
| | | CDR L2 | | DAS |
| | | CDR L3 | 424 | QQTSTIPPT |
| Lib1_P1_A5 | VH | All | 425 | QVQLVQSGAEVKKPGASVKVSCKVS |
| | | | | GDTLSNYAIHWVRQAPGKGLEWMGG |
| | | | | INPNAGTTIYAQKFQGRVTMTEDTST |
| | | | | DTAYMELSSLKSEDTAVYYCARDSSS |
| | | | | WCPVGYYYYYMDVWGKGTTVTVSS |
| | | CDR H1 | 426 | GDTLSNYA |
| | | CDR H2 | 427 | INPNAGTT |
| | | CDR H3 | 428 | ARDSSSWCPVGYYYYYMDV |
| | VL | All | 429 | DIQMTQSPSSVSASVGDRVTITCRASQ |
| | | | | SIGDFLAWYQQKPGKAPKLLIYDASH |
| | | | | LQTGVPSRFSGSGSGTDFTLTISSLQPE |
| | | | | DFANYYCQQTSTIPPTFGGGTKVEIK |
| | | CDR L1 | 430 | QSIGDF |
| | | CDR L2 | | DAS |
| | | CDR L3 | 431 | QQTSTIPPT |
| 9312_Lib8 | VH | All | 432 | EVQLVESGGGLVQPGRSLRLSCAASG |
| | | | | FSLSTYDMHWVRQAPGKGLEWVSAI |
| | | | | SSSGDTRDYADSVEGRFTISRDNAKNS |
| | | | | LYLQMNSLRAEDTAVYYCARAFWGG |
| | | | | DAFDIWGQGTMVTVSS |
| | | CDR H1 | 433 | GFSLSTYD |
| | | CDR H2 | 434 | ISSSGDTR |
| | | CDR H3 | 435 | ARAFWGGDAFDI |
| | VL | All | 436 | DIQMTQSPSSLSASVGDRVTITCRASQ |
| | | | | TIGYWLAWYQQKPGKAPKLLIYGAST |
| | | | | LEGGVPSRFSGSGSGTDFTLTISSLQPE |
| | | | | DVATYYCQHSYSAPPTFGGGTKVEIK |
| | | CDR L1 | 437 | QTIGYW |
| | | CDR L2 | | GAS |
| | | CDR L3 | 438 | QHSYSAPPT |
| 4761_Lib8 | VH | All | 439 | EVQLVESGGGLVQPGRSLRLSCAASG |
| | | | | FTFSSDTMHWVRQAPGKGLEWVSAIS |
| | | | | SSGDHTDYADSVEGRFTISRDNAKNS |
| | | | | LYLQMNSLRAEDTAVYYCAREGLRG |
| | | | | YYFDYWGQGTLVTVSS |
| | | CDR H1 | 440 | GFTFSSDT |
| | | CDR H2 | 441 | ISSSGDHT |
| | | CDR H3 | 442 | AREGLRGYYFDY |
| | VL | All | 443 | DIQMTQSPSSLSASVGDRVTITCRASQ |
| | | | | GISGHLAWYQQKPGKAPKLLIYAASA |
| | | | | LQSGVPSRFSGSGSGTDFTLTISSLQPE |
| | | | | DVATYYCQQSHSLPWTFGGGTKVEIK |
| | | CDR L1 | 444 | QGISGH |
| | | CDR L2 | | AAS |
| | | CDR L3 | 445 | QQSHSLPWT |
| 17561_Lib6 | VH | All | 446 | EVQLVQSGAEVKKPGASVKVSCKAS |
| | | | | GYTFTDLPISWVRQAPGQGLEWMGW |
| | | | | INPYTGDTNYAQKLQGRGTMTTDPST |
| | | | | STAYMELRSLRSDDTAVYYCARDGVI |
| | | | | LGYYYYYGMDVWGQGTTVTVSS |
| | | CDR H1 | 447 | GYTFTDLP |
| | | CDR H2 | 448 | INPYTGDT |
| | | CDR H3 | 449 | ARDGVILGYYYYYGMDV |
| | VL | All | 450 | ESALTQPASVSGSPGQSITISCTGTDLG |
| | | | | SKSVSWYQQHPGKAPKLMIYDNFNRP |
| | | | | SGVSNRFSGSKSGNTASLTISGLQAED |
| | | | | EADYYCSSRTVSGTYVFGGGTKLTVL |
| | | CDR L1 | 451 | DLGSKS |
| | | CDR L2 | | DNF |
| | | CDR L3 | 452 | SSRTVSGTYV |

In some embodiments, an antibody listed in Table 1 is a monovalent (one-arm; “OA”) human antibody in an IgG4 format. In some embodiments, one-arm antibodies of the disclosure include a first polypeptide chain comprising a first heavy chain (HC) region comprising, from N-terminus to C-terminus: a variable heavy chain (VH) region (e.g., any one of the VH regions listed in Table 1), a first heavy chain constant (CH)1 region operably linked (e.g., via a peptide bond) to a first hinge domain at its C-terminus, a first CH2 region operably linked at its N-terminus (e.g., via a peptide bond) to the C-terminus of the first hinge domain, and a first CH3 region operably linked at its N-terminus (e.g., via a peptide bond) to the C-terminus of the first CH2 region. In some embodiments, the first HC region comprises a first CH1-hinge-CH2-CH3 polypeptide comprising an amino acid sequence of SEQ ID NO: 484 or a variant thereof having at least 80% sequence identity to SEQ ID NO: 484.

(SEQ ID NO: 484)
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPPCPAPEAAGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
SCAVKGFYPSDIAVEWESNGQPENNYKTTPPVLDSDGSFFLVSRLTVDK
SRWQEGNVFSCSVMHEALHNHYTQKSLSLSLG

In some embodiments, the first polypeptide chain of the one-arm antibody is operably linked (e.g., via a disulfide bridge) to second polypeptide chain comprising a light chain (LC) region. In some embodiments, the light chain region is a polypeptide comprising, from N-terminus to C-terminus: a VL region (e.g., any one of the VL regions listed in Table 1) operably linked at its C-terminus to the N-terminus of a light chain constant (CL) region. In some embodiments, the CL region is a kappa chain. In some embodiments, the CL region comprises an amino acid sequence of SEQ ID NO: 485 or variant thereof having at least 80% sequence identity to SEQ ID NO: 485.

(SEQ ID NO: 485)
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC

In some embodiments, the VH and VL regions together comprise an antigen binding site that specifically binds to the CK domain of a VWF protein.

In some embodiments, a one-arm antibody of the disclosure further comprises a third polypeptide chain comprising a second HC region, wherein the second HC region does not include a VH region. In some embodiments, the second HC region comprises a second CH1 region operably linked (e.g., via a peptide bond) to a second hinge domain at its C-terminus, a second CH2 region operably linked at its N-terminus (e.g., via a peptide bond) to the C-terminus of the second hinge domain, and a second CH3 region operably linked at its N-terminus (e.g., via a peptide bond) to the C-terminus of the second CH2 region. In some embodiments, the second CH1 region is truncated relative to a CH1 region of the first HC domain (e.g., by 1, 2, 3, 4, 5, 6, 7, 8, 9 10, 11, 12, 13, 14, 16, 17, 18, 19, 20, 21, 22, 23, 24, 25, 26, 27, 28, 29, 30, 31, 32, 33, 34, 35, 36, 37, 38, 39, 40, 41, 42, 43, 44, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more amino acid residues). In some embodiments, the second HC region comprises an amino acid sequence of SEQ ID NO: 473 or is a variant thereof having at least 80% sequence identity to SEQ ID NO: 473.

(SEQ ID NO: 473)
ESKYGPPCPPCPAPEAAGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
WCLVKGFYPSDIAVEWESNGQPENNYKTTPPVLDSDGSFFLYSRLTVDK
SRWQEGNVFSCSVMHEALHNHYTQKSLSLSLG

In some embodiments, the CH1 domain of the HC region is operably linked to the CL region via a disulfide bridge. In some embodiments, the first HC region and the second HC region are operably linked to one another by a disulfide bridge between the first hinge region and the second hinge region.

Accordingly, disclosed herein, in some embodiments, are antibodies comprising an antigen binding domain that specifically binds to the CK domain of a VWF protein, the antigen binding domain comprising: (1) a VH domain (e.g., any one of the VH domains listed in Table 1 or a variant thereof having at least 80% sequence identity thereto), the VH domain being operably linked (e.g., via a peptide bond) at its C-terminus to the N-terminus of a CH1-hinge-CH2-CH3 polypeptide of SEQ ID NO: 484 or a variant thereof having at least 80% sequence identity thereto; and (2) a VL domain (e.g., any one of the VL domains listed in Table 1 or a variant thereof having at least 80% sequence identity thereto) operably linked (e.g., via a peptide bond) at its C-terminus to the N-terminus of a CL region having an amino acid sequence 100% identical to the amino acid sequence of SEQ ID NO: 485 or a variant thereof having at least 80% sequence identity thereto; wherein the antibody further comprises a polypeptide of SEQ ID NO: 473 or a variant thereof having at least 80% sequence identity thereto operably linked (e.g., via a disulfide bridge) to the hinge region of the CH1-hinge-CH2-CH3 polypeptide.

### Binding Epitopes

Antibody epitopes are, in some embodiments, a linear peptide sequence (i.e., “continuous”) or is composed of noncontiguous amino acid sequences (i.e., “conformational” or “discontinuous”). In some embodiments, an antibody recognizes one or more amino acid sequences; therefore, an epitope defines more than one distinct amino acid sequence. Epitopes recognized by antibodies are determined, e.g., by peptide mapping and sequence analysis techniques well known to one of skill in the art. Binding interactions are manifested as intermolecular contacts with one or more amino acid residues of a CDR.

Disclosed herein are antibodies that specifically bind to an epitope of VWF (e.g., human or NHP VWF). In some embodiments, an antibody disclosed herein binds to an epitope comprising noncontiguous amino acid sequences. In some embodiments, a VWF-binding antibody described herein has one antigen-binding site for binding to VWF. In some embodiments, the VWF-binding antibody binds to an epitope in the CK domain of a human VWF protein having an amino acid sequence 100% identical to the amino acid sequence of SEQ ID NO: 5 or a variant thereof having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO: 5.

(SEQ ID NO: 5)
CNDITARLQYVKVGSCKSEVEVDIHYCQGKCASKAMYSIDINDVQDQCS
CCSPTRTEPMQVALHCTNGSVVYHEVLNAMECKCSPRKCSK

In some embodiments, the VWF-binding antibody binds to an epitope in the CK domain of a NHP VWF protein having an amino acid sequence 100% identical to the amino acid sequence of SEQ ID NO: 6 or a variant thereof having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO: 6.

(SEQ ID NO: 6)
CSDITARLQYVKVGSCKSEVEVDIHYCQGKCASKAMYSIDINDVQDQCS
CCSPTRTEPMQVPLHCTNGSVVYHEVLNAMQCECSPRKCSK

The disclosure provides, in certain embodiments, protein constructs containing the CK domain of VWF. In some embodiments, the protein construct contains a human, NHP, canine, or murine CK domain of VWF and, optionally, one or more sequences that facilitates the expression, isolation, purification, and/or detection of the protein construct in vitro, in vivo, or ex vivo. In some embodiments, the protein construct containing a human CK domain is a polypeptide having an amino acid sequence 100% identical to the amino acid sequence of SEQ ID NO: 7 or a variant thereof having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO: 7.

(SEQ ID NO: 7)
MIPARFAGVLLALALILPGTLCAEHHHHHHGSGLNDIFEAQKIEWHEGS
LEVLFQGPEPECNDITARLQYVKVGSCKSEVEVDIHYCQGKCASKAMYS
IDINDVQDQCSCCSPTRTEPMQVALHCTNGSVVYHEVLNAMECKCSPRK
CSK
Sequence Key:
single-underlined residues correspond to the N-terminal signal peptide sequence of VWF; bold residues correspond to a His6 tag (SEQ ID NO: 11); double- underlined residues correspond to a sequence for in vitro biotinylation; bold italic residues correspond to a HRV 3C sequence; and bold underlined residues correspond to the CK domain of human VWF protein as provided in SEQ ID NO: 5.

In some embodiments, the protein construct containing a human CK domain is a polypeptide having an amino acid sequence 100% identical to the amino acid sequence of SEQ ID NO: 486 or a variant thereof having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO: 486.

(SEQ ID NO: 486)
MIPARFAGVLLALALILPGTLCAEHHHHHHGSSAWSHPQFEKGSLEVLF
QGPEPECNDITARLQYVKVGSCKSEVEVDIHYCQGKCASKAMYSIDIND
VQDQCSCCSPTRTEPMQVALHCTNGSVVYHEVLNAMECKCSPRKCSKGG
GGSYPYDVPDYA
Sequence Key:
single-underlined residues correspond to the N-terminal signal peptide sequence of VWF; bold residues correspond to a His6 tag; double-underlined residues correspond to a sequence for in vitro biotinylation; bold italic residues correspond to a HRV 3C sequence; bold underlined residues correspond to the CK domain of human VWF protein as provided in SEQ ID NO: 5; and bold and double-underlined correspond to a C- terminal HA-tag.

In some embodiments, the protein construct containing a NHP CK domain is a polypeptide having an amino acid sequence 100% identical to the amino acid sequence of SEQ ID NO: 8 or a variant thereof having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO: 8.

(SEQ ID NO: 8)
MIPARFAGVLLALALILPGTLCAEHHHHHHGSGLNDIFEAQKIEWHEGS
LEVLFQGPEPECSDITARLQYVKVGSCKSEVEVDIHYCQGKCASKAMYS
IDINDVQDQCSCCSPTRTEPMQVPLHCTNGSVVYHEVLNAMQCECSPRK
CSK
Sequence Key:
single-underlined residues correspond to the N-terminal signal peptide sequence of VWF; bold residues correspond to a His6 tag (SEQ ID NO: 11); double- underlined residues correspond to a sequence for in vitro biotinylation; bold italic residues correspond to a HRV 3C sequence; and bold underlined residues correspond to the CK domain of NHP VWF protein as provided in SEQ ID NO: 6.

In some embodiments, the protein construct containing a NHP CK domain is a polypeptide having an amino acid sequence 100% identical to the amino acid sequence of SEQ ID NO: 487 or a variant thereof having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO: 487.

(SEQ ID NO: 487)
MIPARFAGVLLALALILPGTLCAEHHHHHHGSSAWSHPQFEKGSLEVLF
QGPEPECSDITARLQYVKVGSCKSEVEVDIHYCQGKCASKAMYSIDIND
VQDQCSCCSPTRTEPMQVPLHCTNGSVVYHEVLNAMQCECSPRKCSKGG
GGSYPYDVPDYA
Sequence Key:
single-underlined residues correspond to the N-terminal signal peptide sequence of VWF; bold residues correspond to a His6 tag; double-underlined residues correspond to a sequence for in vitro biotinylation; bold italic residues correspond to a HRV 3C sequence; bold underlined residues correspond to the CK domain of a NHP VWF protein as provided in SEQ ID NO: 6; and bold and double-underlined correspond to a C-terminal HA-tag.

In some embodiments, the protein construct containing a canine CK domain is a polypeptide having an amino acid sequence 100% identical to the amino acid sequence of SEQ ID NO: 9 or a variant thereof having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO: 9.

(SEQ ID NO: 9)
MIPARFAGVLLALALILPGTLCAEHHHHHHGSGLNDIFEAQKIEWHEGS
LEVLFQGPEPECKDIIAKLQRVKVGDCKSEEEVDIHYCEGKCASKAVYS
IHMEDVQDQCSCCSPTQTEPMQVPLRCTNGSLIYHEILNAMQCRCSPRK
CSK
Sequence Key:
single-underlined residues correspond to the N-terminal signal peptide sequence of VWF; bold residues correspond to a His6 tag (SEQ ID NO: 11); double- underlined residues correspond to a sequence for in vitro biotinylation; bold italic residues correspond to a HRV 3C sequence; and bold underlined residues correspond to the CK domain of canine VWF protein.

In some embodiments, the protein construct containing a murine CK domain is a polypeptide having an amino acid sequence 100% identical to the amino acid sequence of SEQ ID NO: 10 or a variant thereof having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO: 10.

(SEQ ID NO: 10)
MIPARFAGVLLALALILPGTLCAEHHHHHHGSGLNDIFEAQKIEWHEGS
LEVLFQGPEPDCKDITAKVQYIKVGDCKSQEEVDIHYCQGKCASKAVYS
IDIEDVQEQCSCCLPSRTEPMRVPLHCTNGSVVYHEVINAMQCRCSPRN
CSK
Sequence Key:
single-underlined residues correspond to the N-terminal signal peptide sequence of VWF; bold residues correspond to a His6 tag (SEQ ID NO: 11); double- underlined residues correspond to a sequence for in vitro biotinylation; bold italic residues correspond to a HRV 3C sequence; and bold underlined residues correspond to the CK domain of murine VWF protein.

In some embodiments, the antibody binds to one or more amino acid residues of the VWF CK domain selected from D2726, R2730, K2735, E2744, D2746, K2757, D2763, and T2789. In some embodiments, the antibody binds to at least the following amino acid residues of the VWF CK domain: D2726, R2730, D2746, and K2757. In some embodiments, the antibody binds to at least the following amino acid residues of the VWF CK domain: R2730, K2735, E2744, and D2746. In some embodiments, the antibody binds to at least the following amino acid residues of the VWF CK domain: K2735, D2746, and K2757. In some embodiments, the antibody binds to at least the following amino acid residues of the VWF CK domain: R2730, D2746, and K2757. In some embodiments, the antibody binds to at least the following amino acid residues of the VWF CK domain: K2735, E2744, D2746, and D2763. In some embodiments, the antibody binds to at least the following amino acid residues of the VWF CK domain: R2730, K2735, E2744, D2746, K2757, and D2763. In some embodiments, the antibody binds to at least amino acid residue K2735 of the VWF CK domain. In some embodiments, the antibody binds to at least the following amino acid residues of the VWF CK domain: D2726, K2735, and K2757. In some embodiments, the antibody binds to at least the following amino acid residues of the VWF CK domain: K2735 and D2763.

Also disclosed herein are additional antibodies that specifically bind to the epitope disclosed herein. These additional antibodies, or antigen-binding fragments thereof that specifically bind to the epitope disclosed herein are, in some embodiments, identified using techniques known in the art. For example, a computational approach is used to design epitope-specific antibodies, as is known in the art. Another approach is used to identify antibodies that bind to specific epitopes from a library of antibodies that bind to the antigen, in some embodiments, such as the following: first incorporate noncanonical amino acids (ncAAs) p-benzoyl-L-phenylalanine (pBpa) and p-azido-L-phenylalanine (pAzF) into the target epitope and then select the antibodies that cross-link with the ncAA incorporated epitope after UV irradiation. Because cross-linking only occurs when the distance between the antibody and the epitope is close enough, this method efficiently selects antibodies that specifically bind to the target epitope.

### Modifications of Antibodies

Antibodies, or antigen-binding fragments thereof, are modified, in some cases, using techniques known in the art for various purposes.

In some embodiments, VWF-binding antibodies of the disclosure contain an Fc region. In some embodiments, the Fc region is a wild-type Fc region. In some embodiments, the Fc region is a modified Fc region. In some cases when an antigen-binding fragment does not contain an Fc region, an Fc region is added to (e.g., recombinantly) the fragment, e.g., to increase half-life of the antigen-binding fragment in circulation in blood when administered to a subject. Choice of an appropriate Fc region and methods of to incorporate such fragments are known in the art. Incorporating an Fc region of an IgG into a polypeptide of interest so as to increase its circulatory half-life, but so as not to lose its biological activity is accomplished, e.g., by using conventional techniques known in the art. In some embodiments, Fc regions of antibodies are further modified to increase half-life of the antigen-binding fragment in circulation in blood when administered to a subject. In some embodiments, an Fc region of an antibody disclosed herein is modified to reduce or silence effector functions of the antibody (e.g., antibody-dependent cellular cytotoxicity (ADCC), complement dependent cytotoxicity (CDC), opsonization, phagocytosis, transcytosis, neutralization of infectivity, inflammation, mucosal immunity, and neonatal immunity). Modifications are, e.g., determined using conventional means in the art. In some embodiments, the modified Fc region comprises one or more mutations selected from the group consisting of a R435H mutation, N434A mutation, T252L/T253S/T254F mutation, E294delta/T307P/N434Y mutation, T256N/A378V/S383N/N434Y mutation, E294 delta mutation, M252Y/S254T/T256E mutation, M428L/N434S mutation, T307A/E380A/N434A mutation, T250Q/M428L mutation, T250Q/M428F mutation, T250E/M428F mutation, T250E/M428L mutation, T256D/Q311V/A378V mutation, T256D/H286D/T307R/Q311V/A378V mutation, H285N/T307Q/N315D mutation, T307Q/Q311V/A378V mutation, H285D/T307Q/A378V mutation, L234F/L235E/P331S mutation, L234F/L235Q/K322Q mutation, L234F/L235Q/P331G mutation, L234F/L235A/K322Q mutation, S228P/F234A/L235A/G237A/P238S mutation, L234A/L235E/G237A/A330S/P331S mutation, F243A/V264A mutation, S228P/L235E/P329G mutation, M252Y/M428L mutation, D2591/V308F mutation, T307Q/N434S mutation, M428L/V308F mutation, Q311V/N434S mutation, H433K/N434F mutation, E258F/V427T mutation, K288E/H435K mutation, F234A/L235A mutation, F234A/L235A/S228P mutation, L234A/L235A/P329G mutation, F234A/L235A/P329G mutation, P329G mutation, F234 mutation, S228P mutation, delta-K447 mutation, L235A mutation, L235E mutation, V308P mutation, V308W mutation, V308Y mutation, V308F mutation, N434S mutation, T307 mutation, Q312A mutation, K322A mutation, P329A mutation, P331A mutation, P238A mutation, D265A mutation, D269A mutation, D270A mutation, N297A mutation, A327Q mutation, P329A mutation, S239A mutation, E294A mutation, Q295A mutation, V303A mutation, A330R mutation, T299A mutation, F234A mutation, T366W mutation, T366S mutation, L368A mutation, Y407V mutation, F405L mutation, K409R mutation, F405L/R409K mutation, and any combination thereof. In some embodiments, the Fc region comprises an N-terminal truncation (e.g., a truncation by 1, 2, 3, 4, 5, 6, 7, 8, 9 10, 11, 12, 13, 14, 16, 17, 18, 19, 20, 21, 22, 23, 24, 25, 26, 27, 28, 29, 30, 31, 32, 33, 34, 35, 36, 37, 38, 39, 40, 41, 42, 43, 44, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 N-terminal residues, or more).

In some embodiments, the first heavy chain (HC; e.g., any one of the “VH_HC” sequences listed in Table 4) comprises a S228P/T366W/F234A/L235A/delta-K447 mutation and the second heavy chain (HC; e.g., any one of the “HC_trc” sequences listed in Table 4) comprises a S228P/T366S/L368A/Y407V/F234A/L235A/delta-K447 mutation. In some embodiments, the first heavy chain comprises a S228P/T366W/delta-K447 mutation and the second heavy chain comprises a S228P/T366S/L368A/Y407V/delta-K447 mutation. In some embodiments, the first heavy chain and the second heavy chain further comprise a M252Y/S254T/T256E mutation.

In some embodiments, antibodies of the disclosure are modified, e.g., by addition of polyethylene glycol (PEG). In some embodiments, PEG modification (PEGylation) leads to one or more of improved circulation time, improved solubility, improved resistance to proteolysis, reduced antigenicity and immunogenicity, improved bioavailability, reduced toxicity, improved stability, and easier formulation.

Glycosylation of immunoglobulins has been shown to have significant effects on their effector functions, structural stability, and rate of secretion from antibody-producing cells. The carbohydrate groups responsible for these properties are generally attached to the constant (C) regions of the antibodies. For example, glycosylation of IgG at asparagine 297 in the C_{H}2 domain is required for full capacity of IgG to activate the classical pathway of complement-dependent cytolysis. Glycosylation of IgM at asparagine 402 in the CH3 domain is necessary for proper assembly and cytolytic activity of the antibody. Removal of glycosylation sites as positions 162 and 419 in the C_{H}1 and C_{H}3 domains of an IgA antibody led to intracellular degradation and at least 90% inhibition of secretion.

Glycosylation of immunoglobulins in the variable (V) region has also been observed. About 20% of human antibodies are glycosylated in the V region. Glycosylation of the V domain is believed to arise from fortuitous occurrences of the N-linked glycosylation signal Asn-Xaa-Ser/Thr in the V region sequence and has not been recognized in the art as playing a role in immunoglobulin function.

Glycosylation at a variable domain framework residue, in some cases, alters the binding interaction of the antibody with antigen. The present disclosure includes criteria by which a limited number of amino acids in the framework or CDRs of a humanized immunoglobulin chain are chosen to be mutated (e.g., by substitution, deletion, or addition of residues) to increase the affinity of an antibody. In some embodiments, cysteine residue(s) are removed or introduced in the Fc region of an antibody or Fc-containing polypeptide, thereby eliminating or increasing interchain disulfide bond formation in this region.

It has been shown that sequences within the CDR cause an antibody to bind to MHC Class II and trigger an unwanted helper T-cell response in some cases. In some embodiments, a conservative substitution allows the antibody to retain binding activity yet reduce its ability to trigger an unwanted T-cell response. In one embodiment, one or more of the N-terminal 20 amino acids of the heavy or light chain is removed.

Covalent modifications of an antibody are also included herein. In some embodiments, they are made by chemical synthesis or by enzymatic or chemical cleavage of the antibody, if applicable. In some embodiments, other types of covalent modifications are introduced by reacting targeted amino acid residues with an organic derivatizing agent that is capable of reacting with selected side chains or the N- or C-terminal residues.

Cysteinyl residues are most commonly reacted with alpha-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, alpha-bromo-beta-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

In some embodiments, histidyl residues are derivatized by reaction with diethylpyrocarbonate at pH 5.5-7.0 because this agent is relatively specific for the histidyl side chain. In some embodiments, para-bromophenacyl bromide also is useful; the reaction, in some embodiments, is performed in 0.1 M sodium cacodylate at pH 6.0.

In some embodiments, lysinyl and amino-terminal residues are reacted with succinic or other carboxylic acid anhydrides. Derivatization with these agents has the effect of reversing the charge of the lysinyl residues. Other suitable reagents for derivatizing alpha-amino-containing residues include imidoesters such as methyl picolinimidate, pyridoxal phosphate, pyridoxal, chloroborohydride, trinitrobenzenesulfonic acid, O-methylisourea, 2,4-pentanedione, and transaminase-catalyzed reaction with glyoxylate.

In some embodiments, arginyl residues are modified by reaction with one or several conventional reagents, such as phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents, in some embodiments, react with the groups of lysine as well as the arginine epsilon-amino group.

In some embodiments, the specific modification of tyrosyl residues are made, with particular interest in introducing spectral labels into tyrosyl residues by reaction with aromatic diazonium compounds or tetranitromethane. Most commonly, N-acetylimidazole and tetranitromethane are used to form 0-acetyl tyrosyl species and 3-nitro derivatives, respectively, in some embodiments. Tyrosyl residues are iodinated using ¹²⁵I or ¹³¹I to prepare labeled proteins for use in radioimmunoassay.

Carboxyl side groups (aspartyl or glutamyl) are specifically modified by reaction with carbodiimides (R—N═C═N—R′), where R and R′ are different alkyl groups, such as 1-cyclohexyl-3-(2-morpholinyl-4-ethyl) carbodiimide or 1-ethyl-3-(4-azonia-4,4-dimethylpentyl)carbodiimide. Furthermore, aspartyl and glutamyl residues are converted to asparaginyl and glutaminyl residues by reaction with ammonium ions.

In some embodiments, glutaminyl and asparaginyl residues are deamidated to the corresponding glutamyl and aspartyl residues, respectively. These residues are deamidated under neutral or basic conditions.

Other modifications include hydroxylation of proline and lysine, phosphorylation of hydroxyl groups of seryl or threonyl residues, methylation of the alpha-amino groups of lysine, arginine, and histidine side chains, acetylation of the N-terminal amine, and amidation of any C-terminal carboxyl group.

Another type of covalent modification involves chemically or enzymatically coupling glycosides to the specific binding agent or antibody. These procedures do not require production of the polypeptide or antibody in a host cell that has glycosylation capabilities for N- or O-linked glycosylation. Depending on the coupling mode used, in some embodiments, the sugar(s) are attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine.

Removal of any carbohydrate moieties present on the polypeptide or antibody are, in some embodiments, accomplished chemically or enzymatically. Chemical deglycosylation involves exposure of the antibody to the compound trifluoromethanesulfonic acid, or an equivalent compound. This treatment results in the cleavage of most or all sugars except the linking sugar (N-acetylglucosamine or N-acetylgalactosamine), while leaving the antibody intact. Enzymatic cleavage of carbohydrate moieties on an antibody is achieved by the use of a variety of endo- and exo-glycosidases in some embodiments.

Another type of covalent modification comprises linking an antibody to one of a variety of nonproteinaceous polymers, e.g., polyethylene glycol, polypropylene glycol, polyoxyethylated polyols, polyoxyethylated sorbitol, polyoxyethylated glucose, polyoxyethylated glycerol, polyoxyalkylenes, or polysaccharide polymers such as dextran. Such methods are known in the art.

Affinity for binding a pre-determined polypeptide antigen, generally, is modulated by introducing one or more mutations into the V region framework, typically in areas adjacent to one or more CDRs and/or in one or more framework regions. Typically, such mutations involve the introduction of conservative amino acid substitutions that either destroy or create the glycosylation site sequences but do not substantially affect the hydropathic structural properties of the polypeptide. Typically, mutations that introduce a proline residue are avoided.

In some embodiments, the antibody or antigen-binding fragment provided herein is conjugated or linked to a therapeutic moiety, an imaging or detectable moiety, or an affinity tag. Methods for conjugating or linking polypeptides are well known in the art. Associations (binding) between compounds and labels include any means known in the art including, but not limited to, covalent and non-covalent interactions, chemical conjugation, as well as recombinant techniques. An antibody or antigen-binding fragment thereof is conjugated to, or recombinantly engineered with, an affinity tag (e.g., a purification tag), in some embodiments. Affinity tags such as, e.g., poly-histidine tags (e.g., His6 (SEQ ID NO: 11)) are conventional in the art.

In some embodiments, the antibody or antigen-binding fragment further comprises a detectable moiety. Detections accomplished, e.g., in vitro, in vivo or ex vivo. In vitro assays for the detection and/or determination (quantification, qualification, etc.) of, e.g., VWF protein levels using the antibodies or antigen-binding fragments thereof include but are not limited to, e.g., ELISAs, RIAs, and western blots. In some embodiments, in vitro detection, diagnosis, or monitoring of the antigen of the antibodies occurs by obtaining a sample (e.g., a blood sample) from a subject and testing the sample in, e.g., a standard ELISA assay.

### Pharmaceutical Compositions

Disclosed herein, in certain embodiments, are pharmaceutical compositions comprising an antibody as disclosed herein and a pharmaceutically acceptable carrier, diluent, or excipient. Such compositions are useful for in vitro or in vivo analysis or, in the case of pharmaceutical compositions, for administration to a subject in vivo or ex vivo for treating a subject with the disclosed antibodies.

In some embodiments, the carrier, diluent, or excipient is a stabilizer, buffer, surfactant, filler, solvent, tonicity or osmolarity adjusting agent, antioxidant, adjuvant, and antimicrobial agent or other suitable materials known to those skilled in the art. Such materials should be non-toxic and should not interfere with the efficacy of the active ingredient. The precise nature of the carrier or other material will depend on the route of administration.

Pharmaceutical formulations comprising an antibody or antigen-binding fragment, identified by the methods described herein are prepared for storage by mixing the protein having the desired degree of purity with optional physiologically acceptable carrier, diluent, or excipient (see, e.g., Remington's Pharmaceutical Sciences, 16^{th} edition, Osol, A. Ed. (1980)), in the form of lyophilized formulations or aqueous solutions in some embodiments. Acceptable carrier, diluent, or excipient are those that are non-toxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG). In certain embodiments, the pharmaceutical composition comprises the antibody at a concentration of between 5-200 mg/mL. In some embodiments, the pharmaceutical composition comprises the antibody at a concentration of 150 mg/mL. In some embodiments, the pharmaceutical composition is in a volume of no greater than 1.0 mL (e.g., 1.0 mL, 0.9 mL, 0.8 mL, 0.7 mL, 0.6 mL, 0.5 mL, 0.4 mL, 0.3 mL, 0.2 mL, 0.1 mL, or less). In some embodiments, the pharmaceutical composition is stable as a liquid solution at room temperature.

Acceptable carriers are physiologically acceptable to the administered subject and retain the therapeutic properties of the compounds with/in which it is administered. Acceptable carriers and their formulations are and generally described in, e.g., Remington's Pharmaceutical Sciences, supra. One exemplary carrier is physiological saline. The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject compounds from the administration site of one organ, or portion of the body, to another organ, or portion of the body, or in an in vitro assay system. Each carrier is acceptable in the sense of being compatible with the other ingredients of the formulation and not injurious to a subject to whom it is administered. Nor should an acceptable carrier alter the specific activity of the subject compounds.

In another embodiment, a pharmaceutical composition disclosed herein further comprises an acceptable additive to improve the stability of the compounds in composition and/or to control the release rate of the composition. Acceptable additives do not alter the specific activity of the subject compounds. Exemplary acceptable additives include, but are not limited to, a sugar such as mannitol, sorbitol, glucose, xylitol, trehalose, sorbose, sucrose, galactose, dextran, dextrose, fructose, lactose, and mixtures thereof. Acceptable additives are combined with acceptable carriers and/or excipients such as dextrose, in some embodiments. Alternatively, exemplary acceptable additives include, but are not limited to, a surfactant such as polysorbate 20 or polysorbate 80 to increase stability of the peptide and decrease gelling of the solution. In some embodiments, the surfactant is added to the composition in an amount of 0.01% to 5% of the solution. Addition of such acceptable additives increases the stability and half-life of the composition in storage.

In one embodiment, a pharmaceutical composition disclosed herein contains an isotonic buffer such as a phosphate, acetate, or TRIS buffer in combination with a tonicity agent such as a polyol, sorbitol, sucrose or sodium chloride, which tonicifies and stabilizes. In some embodiments, a tonicity agent is present in the composition in an amount of about 5%.

In another embodiment, a pharmaceutical composition disclosed herein includes a surfactant such as to prevent aggregation and for stabilization at 0.01 to 0.02% w/v.

In another embodiment, the pH of a pharmaceutical composition disclosed herein ranges from 4.5-6.5 or 4.5-5.5.

In some embodiments, a pharmaceutical composition disclosed herein also contains more than one active compound as necessary for the indication being treated, such as those with complementary activities that do not adversely affect each other. Such molecules are suitably present in combination in amounts that are effective for the purpose intended.

In some embodiments, active ingredients are entrapped in microcapsule prepared, e.g., by coacervation techniques or by interfacial polymerization, e.g., hydroxy methylcellulose or gelatin-microcapsule and poly-(methylmethacrylate) microcapsule, respectively, in colloidal drug delivery systems (e.g., liposomes, albumin microspheres, microemulsions, nanoparticles, and nanocapsules) or in macroemulsions. Such techniques are disclosed in Remington's Pharmaceutical Sciences, supra.

Suspensions and crystal forms of antibodies are also contemplated herein; methods to make suspensions and crystal forms are known to one of skill in the art.

In some embodiments, a pharmaceutical composition disclosed herein is sterile. In some embodiments, a pharmaceutical composition disclosed herein is sterilized by conventional, well known sterilization techniques. For example, sterilization is readily accomplished by filtration through sterile filtration membranes. In some embodiments, the resulting solutions is packaged for use or filtered under aseptic conditions and lyophilized, the lyophilized preparation being combined with a sterile solution prior to administration.

Freeze-drying is employed to stabilize polypeptides for long-term storage, such as when a polypeptide is relatively unstable in liquid compositions, in some embodiments.

In some embodiments, excipients such as, e.g., polyols (including mannitol, sorbitol, and glycerol), sugars (including glucose and sucrose), and amino acids (including alanine, glycine, and glutamic acid) act as stabilizers for freeze-dried products. Polyols and sugars are also used to protect polypeptides from freezing and drying-induced damage and to enhance the stability during storage in the dried state in some embodiments. Sugars are, in some embodiments, effective in both the freeze-drying process and during storage. Other classes of molecules, including mono- and disaccharides and polymers, such as PVP, have also been reported as stabilizers of lyophilized products.

For injection, in some embodiments, a pharmaceutical composition disclosed herein is a powder suitable for reconstitution with an appropriate solution as described above. Examples of these include, but are not limited to, freeze dried, rotary dried, or spray dried powders, amorphous powders, granules, precipitates, or particulates. For injection, the compositions optionally contain stabilizers, pH modifiers, surfactants, bioavailability modifiers, and combinations of these.

Sustained-release preparations are prepared, in some embodiments. Suitable examples of sustained-release preparations include semipermeable matrices of solid hydrophobic polymers containing the antibody, which matrices are in the form of shaped articles, e.g., films, or microcapsule. Examples of sustained-release matrices include polyesters, hydrogels (e.g., poly(2-hydroxyethyl-methacrylate), or poly(vinylalcohol)), polylactides (see, e.g., U.S. Pat. No. 3,773,919), copolymers of L-glutamic acid and y ethyl-L-glutamate, non-degradable ethylene-vinyl acetate, degradable lactic acid-glycolic acid copolymers such as the Lupron Depot™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. In some embodiments, while encapsulated antibodies remain in the body for a long time, they denature or aggregate as a result of exposure to moisture at 37° C., resulting in a loss of biological activity and possible changes in immunogenicity. Rational strategies devised for stabilization are, in some cases, dependent on the mechanism involved. For example, if the aggregation mechanism is discovered to be intermolecular S— bond formation through thio-disulfide interchange, stabilization is achieved, in some cases, by modifying sulfhydryl residues, lyophilizing from acidic solutions, controlling moisture content, using appropriate additives, and developing specific polymer matrix compositions.

In some embodiments, a pharmaceutical composition disclosed herein is designed to be short-acting, fast-releasing, long-acting, or sustained-releasing as described herein. In one embodiment, a pharmaceutical composition disclosed herein is formulated for controlled release or for slow release.

The pharmaceutical composition is administered, e.g., by injection, including, but not limited to, subcutaneous, intravitreal, intradermal, intravenous, intra-arterial, intraperitoneal, intracerebrospinal, or intramuscular injection. Excipients and carriers for use in formulation of compositions for each type of injection are contemplated herein. The following descriptions are by example only and are not meant to limit the scope of the compositions. Compositions for injection include, but are not limited to, aqueous solutions (where water soluble) or dispersions, as well as sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In some embodiments, the carrier is a solvent or dispersion medium containing, e.g., water, ethanol, polyol (e.g., glycerol, propylene glycol, and liquid polyethylene glycol, and the like), and suitable mixtures thereof. Fluidity is maintained, e.g., by the use of a coating such as lecithin, by the maintenance of the required particle size in the case of dispersion and by the use of surfactants. Antibacterial and antifungal agents include, e.g., parabens, chlorobutanol, phenol, ascorbic acid, and thimerosal. Isotonic agents, e.g., sugars, polyalcohols such as mannitol, sorbitol, and sodium chloride are included in the composition in some embodiments. In some embodiments, the resulting solutions are packaged for use as is, or lyophilized; the lyophilized preparation is later combined with a sterile solution prior to administration, in some embodiments. For intravenous injection or injection at the site of affliction, the active ingredient will be in the form of a parenterally acceptable aqueous solution which is pyrogen-free and has suitable pH, isotonicity, and stability. Those of relevant skill in the art are well able to prepare suitable solutions using, e.g., isotonic vehicles such as Sodium Chloride Injection, Ringer's Injection, and Lactated Ringer's Injection. Preservatives, stabilizers, buffers, antioxidants, and/or other additives are included as needed, in some embodiments. Sterile injectable solutions are prepared by incorporating an active ingredient in the required amount in an appropriate solvent with one or a combination of ingredients enumerated above, as required, followed by filtered sterilization, in some embodiments. Generally, dispersions are prepared by incorporating the active ingredient into a sterile vehicle which contains a basic dispersion medium and the required other ingredients from those enumerated above. In the case of sterile powders for the preparation of sterile injectable solutions, the preferred methods of preparation are vacuum drying and freeze drying which yields a powder of the active ingredient plus any additional desired ingredient from a previously sterile-filtered solution thereof.

Compositions are conventionally administered intravenously in some embodiments, such as by injection of a unit dose. For injection, in some embodiments, an active ingredient is in the form of a parenterally acceptable aqueous solution which is substantially pyrogen-free and has suitable pH, isotonicity, and stability. In some embodiments, one prepares suitable solutions using, e.g., isotonic vehicles such as Sodium Chloride Injection, Ringer's Injection, Lactated Ringer's Injection. Preservatives, stabilizers, buffers, antioxidants, and/or other additives are included, as required, in some embodiments. Additionally, compositions are administered via aerosolization in some embodiments.

For parenteral administration, the antibodies are formulated in a unit dosage injectable form (e.g., solution, suspension, or emulsion) in association with a pharmaceutically acceptable, parenteral vehicle. Examples of such vehicles are water, saline, Ringer's solution, dextrose solution, and 5% human serum albumin. Nonaqueous vehicles such as fixed oils and ethyl oleate are also used. In some embodiments, liposomes are used as carriers. The vehicle contains minor amounts of additives such as substances that enhance isotonicity and chemical stability, e.g., buffers and preservatives. The antibodies are typically formulated in such vehicles at concentrations of about 1 mg/mL to 10 mg/mL.

In one embodiment, a pharmaceutical composition disclosed herein is lyophilized, e.g., to increase shelf-life in storage. When the compositions are considered for use in medicaments or any of the methods provided herein, in some embodiments, it is contemplated that the composition are substantially free of pyrogens such that the composition will not cause an inflammatory reaction or an unsafe allergic reaction when administered to a human subject. Testing compositions for pyrogens and preparing compositions substantially free of pyrogens are well understood to one or ordinary skill of the art and are accomplished using commercially available kits in some embodiments.

In some embodiments, acceptable carriers contain a compound that stabilizes, increases, or delays absorption or clearance. Such compounds include, e.g., carbohydrates, such as glucose, sucrose, or dextrans; low molecular weight proteins; compositions that reduce the clearance or hydrolysis of peptides; or excipients or other stabilizers and/or buffers. Agents that delay absorption include, e.g., aluminum monostearate and gelatin. In some embodiments, detergents also be used to stabilize or to increase or decrease the absorption of the pharmaceutical composition, including liposomal carriers. To protect from digestion the compound, in some embodiments, is complexed with a composition to render it resistant to acidic and enzymatic hydrolysis, or the compound is, in some embodiments, complexed in an appropriately resistant carrier such as a liposome. Means of protecting compounds from digestion are known in the art.

### Methods of Treatment

Disclosed herein, in certain embodiments, are methods of treating a blood disorder characterized by excessive bleeding in a subject in need thereof, comprising administering to the subject an antibody or pharmaceutical composition containing the antibody disclosed herein. In some embodiments, the disclosure provides a use of an antibody as described herein for the manufacture of a medicament. In some embodiments, the disclosure provides a use of an antibody as described herein for the treatment of a blood disorder characterized by excessive bleeding.

Blood disorders characterized by excessive bleeding arise, in part, due to dysregulation of hemostasis, which can occur when proteins involved in the clotting signaling cascades are disrupted. For example, von Willebrand disease (VWD) is an inherited bleeding disorder that results from deficiency in VWF. Such deficiency can arise from low levels of VWF (i.e., quantitative effect) or from presence of dysfunctional VWF (i.e., qualitative effect). It is one of the most common inherited blood clotting disorders in humans. Hereditary forms of VWD include type 1, type 2, type 3, and platelet-type VWD. In addition to inherited pathology, VWD exists in acquired forms. Generally, VWD is characterized by tendency to bleed, easy bruising, nosebleeds, bleeding gums, heavy menstrual bleeding, and blood loss during childbirth. Recently, another diagnostic category of “low VWF” has emerged, corresponding to individuals with VWF levels that are below normal but insufficient to meet diagnostic criteria for VWD. VWF deficiency in VWD presents primarily in organs with small vessels, including skin, gastrointestinal tract, uterus, and colon.

As mentioned above, hereditary VWD occurs in multiple forms, including type 1, type 2, type 3, and platelet-type. Type 1 VWD is the predominant form (40-80% of all cases), which presents as VWF levels that are less than 50% of levels in healthy subjects. Type 1 VWD patients can be asymptomatic and lead a normal life without complications. Therefore, although VWD occurs in about 1% of the population, clinically significant cases are observed in about 1/10,000 cases. Type 2 VWD corresponds to 15-50% of all cases and generally presents as a bleeding tendency that varies between patients. Type 2 VWD can be further classified into subtypes based on the presence and behavior of VWF multimers, including Types 2A, 2B, 2M, and 2N. Type 2A is characterized by a defect in the ability to form large VWF multimers. Type 2B is characterized by a gain of function in which the binding between VWF and platelet gp1b receptor is strongly enhanced, resulting in abnormal platelet binding and rapid clearance of platelet-VWF multimer complexes. This leads to reduced or absent levels of large VWF multimers. Type 2M VWD results from reduced binding between VWF and gp1b platelet receptor, resulting in reduced platelet adhesion and aggregation. Type 2N VWD corresponds to a deficiency in binding between VWF and FVIII, resulting in reduced blood plasma levels of FVIII similar to those seen in hemophilia A. Type 3 VWD is characterized by homozygosity for the defective VWF gene and features complete absence of VWF in plasma, resulting in the most severe form of VWD. Type 3 VWD is also associated with very low blood plasma levels of FVIII. Platelet-type VWD presents with qualitatively and quantitatively normal VWF protein, but features a defect in the platelet gp1b receptor that results in increased affinity to VWF. This increased affinity to VWF produces abnormally large platelet aggregates that are cleared from circulation along with VWF multimers. The pathogenesis of acquired VWD is variable. It can be associated with the presence of autoantibodies to VWF, resulting in rapid clearance of VWF-autoantibody complexes from the circulation, or interference with its function. Acquired VWD can also occur with adsorption of VWF by tumor cells and non-immunologic mechanisms of destruction. Current treatments for VWD include use of desmopressin, tranexamic acid (TXA), and concentrates of plasma-derived VWF and/or FVIII or recombinant VWF. However, these therapies suffer from several shortcomings, including but not limited to short half-lives not suitable for bleeding prophylaxis, increased thrombotic and cardiovascular risk, gastrointestinal side effects, limited efficacy in major bleeds, high cost (e.g., VWF/FVIII supplementation), inconvenient intravenous administration (e.g., VWF and FVIII supplementation), and/or risk of blood-transmitted diseases. Therefore, there is an unmet need for the treatment of VWF deficiency that cannot be remedied by currently available therapies.

Hemophilia A is another disease characterized by disrupted hemostasis resulting from a genetic deficiency in functional FVIII. Such a deficiency can arise from low levels of normal FVIII protein and/or the presence of a dysfunctional variant of FVIII. Hemophilia A presents with internal or external bleeding episodes, with bleeding severity corresponding to the degree of FVIII deficiency. Patients with hemophilia A have an elevated risk for excessive bleeding from common injuries and often exhibit spontaneous bleeding without cause. Sites of bleeding may include skin, joints, muscles, digestive tract, and brain. Various mutations in FVIII can result in a positive diagnosis, with the severity of disease resulting from differences in amount of functional FVIII protein. Patients with less than 1% of normal levels of active FVIII exhibit severe hemophilia, those with 1-5% exhibit moderate hemophilia, and those with 5-40% have mild hemophilia.

Heydes syndrome is a syndrome featuring abnormal hemostasis from acquired VWD and gastrointestinal bleeding from angiodysplasia, in the presence of aortic stenosis. Patients with aortic valve stenosis have a narrowed valve, resulting in increased sheer stress on blood passing through it. The increase in shear stress causes platelet and VWF activation, further resulting in the activation of ADAMTS13 and degradation of VWF. Accordingly, the reduced levels of VWF in the blood increase the propensity to bleed. Surgical correction of aortic stenosis results in resolution of the acquired VWD in most cases, but not all patients are suitable for surgery.

Abnormal uterine bleeding (AUB) is a disorder featuring vaginal bleeding from the uterus that is frequent, long-lasting, heavier than normal, and/or irregular, which may lead to anemia and reduced quality of life. AUB affects as many as 25% of women and may be associated with structural and non-structural causes. Examples of structural causes for AUB include leiomyoma (fibroids), adenomyosis (endometrial invasion of myometrium), polyps, and cancer. Non-structural causes for AUB may be medical (e.g., hypothyroidism) or may result from a disorder of hemostasis (accounting for about 10% of all AUB cases). Due to its heterogenous etiology, treatment of AUB is complicated, but generally involves use of hormonal therapies, non-steroidal anti-inflammatory drugs (NSAIDS), and TXA. A recent UK survey of AUB patients (Cox et al., BMC Health Serv Res, 13:491 (2013)) has reported a low rate of satisfaction with treatment outcomes after one year, highlighting an unmet need for improved therapies to treat AUB.

Alpha-2-antiplasmin (A2AP/SERPINF2) deficiency is yet another blood disorder characterized by excessive bleeding. A2AP is a serine protease inhibitor that functions in inactivating plasmin and thereby inhibits fibrinolysis. A2AP deficiency is an especially rare disorder with a prevalence of less than 1 in 1,000,000 that occurs in a homozygous or heterozygous form. In the homozygous form, A2AP deficiency presents with severe bleeding characterized by spontaneous hemorrhage and hemarthrosis. The heterozygous form has variable presentation that can be associated with bleeding post-surgery/injury, and spontaneous musculoskeletal bleeds. No effective, long-lasting prophylactic treatments exist for the treatment of A2AP deficiency.

Disrupted hemostasis can also result from disorders of platelet function. As discussed above, platelet activation, adhesion, and aggregation are critical steps in clot formation and stabilization. Abnormal levels and/or functioning of platelets can result in excessive bleeding (e.g., epistaxis, nosebleeds, gum bleeding, menorrhagia, and prolonged bleeding after cuts or surgery). Platelet disorders can be heritable or acquired. Examples of congenital disorders of platelet function include but are not limited to Glanzmann's thrombasthenia, Bernard-Soulier syndrome, Chromosome 22q11.2 deletion syndromes, platelet-type VWF (PT-VWD), gray platelet syndrome, May-Hegglin anomaly, Epstein syndrome, Fechter syndrome, Sebastian syndrome, Wiskott-Aldrich syndrome, Chediak-Higashi syndrome, Hermansky-Pudlak syndrome, and storage pool/secretion disorder. Non-limiting examples of acquired disorders of platelet function include use of certain medications (e.g., aspirin, dipyridamole, clopidogrel, and ibuprofen), liver disease, uremia, and myeloproliferative disorders (e.g., essential thrombocythemia).

Abnormal bleeding can also be hallmarks of certain heritable connective tissue disorders, resulting from abnormal vasculature with increased vulnerability to injury and abnormal interactions with platelets and coagulation factors. Examples of such disorders include hereditary hemorrhagic telangiectasia (HHT)/Osler-Weber-Rendu (OWR) disease, which is characterized by formation of anteriovenous malformations (AVM) in internal organs, and telangiectasia on mucocutaneous surfaces including skin, gastrointestinal mucosa, and upper respiratory tract. HHT features an autosomal dominant pattern of inheritance and has a prevalence of 1/5000. The majority of HHT patients (80%) have an identifiable causative mutation in ENG, ACVRL1, or MADH4 genes, all of which are involved in TGF-β/BMP signaling pathway required for smooth muscle differentiation and vascular modeling. Mutations in each gene are distinctly associated with particular clinical subtypes of HHT. Specifically, HHT Type 1, accounting for about 60% of all HHT cases, is associated with mutations in ENG (chromosome 9q34.11) and presents with AVM in lungs and brain. HHT Type 2, accounting for about 38% of all HHT cases, is associated with mutations in ACVRL1 (chromosome 12q13.13) and presents with AVM in liver, lungs, and spine. The third subtype, HHT with juvenile polyposis coli, is associated with mutations in MADH4 (chromosome 18q21.2), presents with AVM and gastrointestinal polyps, and accounts for about 2% of all HHT cases. Among HHT patients, clinical presentation is highly variable and is associated with an increased risk for cerebral abscess, migraine, ischemic/embolic stroke, heart failure, colon cancer, and bleeding complications such as, e.g., epistaxis, gastrointestinal bleeding, hemorrhagic stroke secondary to CNS malformation, pulmonary hemorrhage, increased risk of mortality in pregnancy associated with pulmonary hemorrhage and CNS bleeds, and iron deficiency. Available treatment options for HHT include invasive techniques such as surgery cryotherapy, and ablation, treatment with TXA, hormonal therapy (e.g., estrogen, tamoxifen, or danazol), or an anti-angiogenic therapy (e.g., thalidomide and bevacizumab).

Deficiency in anti-coagulation factors can also result in abnormal hemostasis. Loss of regulation of these factors results in an increased tendency to form a thrombus. For example, congenital deficiency in AT is an autosomal dominant disease state characterized by increased propensity for clotting, resulting in a 5-50 fold increase in the risk of thrombosis. Congenital AT deficiency is reported to occur with a prevalence of 1:500 to 1:5000. Two subtypes of AT have been identified, Type 1 (quantitative defect) and Type 2 (qualitative defect). Type 1 AT deficiency is associated with a decrease in AT activity and blood concentration and can be further divided into subgroup 1a, featuring normal affinity for heparin, and subgroup 1b which features reduced affinity for heparin. Type 1 AT deficiency are predominately due to point mutations, deletions, or insertions within the AT gene. Type 2 AT deficiency is associated with normal AT levels, but reduced AT activity and is further divided into 3 subgroups, namely subgroup 2a, featuring decreased thrombin inactivation, FXa inactivation, and heparin affinity, subgroup 2b, featuring decreased thrombin inactivation and normal heparin affinity, and subgroup 2c, featuring normal thrombin inactivation and Xa inactivation and decreased heparin affinity. AT deficiency can also be acquired by one of three ways: (1) increased AT excretion, e.g., in renal failure associated with proteinuria nephrotic syndrome or in burns; (2) decreased AT production, e.g., as observed in liver dysfunction; or (3) accelerated consumption of AT, e.g., as a result of injury or invasive surgical procedures. Current therapies for congenital AT deficiency rely on anticoagulation therapies, however, these treatments are associated with an increased risk of spontaneous hemorrhage. Other therapies include use of low molecular weight heparin with addition of AT concentrate to overcome heparin resistance. However, AT concentrate is administered as an intravenous infusion, making it suitable only for short-term treatments (e.g., peri-operative bridging).

In some embodiments, a blood disorder characterized by excessive bleeding is selected from a von Willebrand disease (VWD), hemophilia A, disorders of platelet function, and connective tissue disorders. In some embodiments, a blood disorder characterized by excessive bleeding is VWD. In some embodiments, the VWD is congenital VWD (cVWD). In some embodiments, the VWD is acquired VWD (aVWD). In some embodiments, the aVWD includes Heydes syndrome. In some embodiments, the VWD is type 1 VWD. In some embodiments, the VWD is type 2A VWD. In some embodiments, the VWD is type 2B VWD. In some embodiments, the VWD is type 2M VWD. In some embodiments, the VWD is type 2N VWD. In some embodiments, the VWD is type 3 VWD. In some embodiments, a blood disorder characterized by excessive bleeding is hemophilia A. In some embodiments, a blood disorder characterized by excessive bleeding is a disorder of platelet function. In some embodiments, a disorder of platelet function is Glanzmann's thrombasthenia. In some embodiments, a disorder of platelet function is Bernard-Soulier syndrome. In some embodiments, a disorder of platelet function is a chromosome 22q11.2 deletion syndrome. In some embodiments, a disorder of platelet function is platelet type VWD. In some embodiments, a disorder of platelet function is gray platelet syndrome. In some embodiments, a disorder of platelet function is May-Hegglin anomaly. In some embodiments, a disorder of platelet function is Epstein syndrome. In some embodiments, a disorder of platelet function is Fechter syndrome. In some embodiments, a disorder of platelet function is Sebastian syndrome. In some embodiments, a disorder of platelet function is Wiskott-Aldrich syndrome. In some embodiments, a disorder of platelet function is Chediak-Higashi syndrome. In some embodiments, a disorder of platelet function is Hermansky-Pudlak syndrome. In some embodiments, a disorder of platelet function is a storage pool/secretion disorder. In some embodiments, a disorder of platelet function is an acquired disorder of platelet function resulting from use of certain medications (e.g., aspirin, dipyridamole, clopidogrel, and ibuprofen. In some embodiments, a disorder of platelet function is an acquired disorder of platelet function due to liver disease. In some embodiments, a disorder of platelet function is an acquired disorder of platelet function due to uremia. In some embodiments, a disorder of platelet function is an acquired disorder of platelet function due to a myeloproliferative disorder (e.g., essential thrombocythemia). In some embodiments, a disorder of platelet function is idiopathic, but evident in one or more assays used to assess platelet function. In some embodiments, a blood disorder characterized by excessive bleeding is a connective tissue disorder. In some embodiments, a connective tissue disorder is HHT. In some embodiments, the HHT is type 1 HHT. In some embodiments, the HHT is type 2 HHT. In some embodiments, the HHT is HHT with juvenile polyposis coli.

The antibody or the pharmaceutical composition disclosed herein are, in some embodiments, administered to the subject in need thereof in an amount and for a time sufficient to treat one or more symptoms selected from the group consisting of epistaxis, cutaneous bleeding, bleeding from minor wounds, oral-cavity bleeding, angiodysplasia, gastrointestinal bleeding, bleeding from tooth extraction, postoperative bleeding, heavy menstrual bleeding, obstetric hemorrhage, hematuria, muscle hematoma, joint bleeding, visceral bleeding, and central nervous system (CNS) bleeding.

Disclosed herein, in certain embodiments, are methods of accumulating VWF protein in blood plasma of a subject in need thereof, comprising administering to the subject a composition comprising an antibody or pharmaceutical composition disclosed herein. In some embodiments, the accumulation of the VWF protein results in a blood plasma level of the VWF protein that is at least 1.3-fold, 1.4-fold, 1.5-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold greater as compared to a blood plasma level of the VWF protein in the absence of the antibody. In some embodiments, the accumulation of the VWF protein results in a blood plasma level of the VWF protein that is at least 5%, 10%, 20%, 30%, 40%, 50%, 60%, 70%, 80%, 90%, 100%, 150%, 200%, 250%, 300%, 350%, 400%, 450%, 500%, 550%, 600%, 650%, 700%, 750%, 800%, 850%, 900%, 950%, 1,000%, 1,500%, 2,000%, 2,500%, 3,000%, 3,500%, 4,000%, 4,500%, 5,000%, 5,500%, 6,000%, 6,500%, 7,000%, 7,500%, 8,000%, 8,500%, 9,000%, 9,500%, 10,000%, or greater as compared to a blood plasma level of the VWF protein in the absence of the antibody. In some embodiments, the degree of accumulation of a VWF protein depends on the effective amount of an antibody or antigen-binding fragment thereof administered to a subject, as described herein.

Disclosed herein, in certain embodiments, are methods of accumulating Factor VIII protein in blood plasma of a subject in need thereof, comprising administering to the subject a composition comprising an antibody or pharmaceutical composition disclosed herein. In some embodiments, the accumulation of the Factor VIII protein results in a blood plasma level of the Factor VIII protein that is at least 1.5-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold greater as compared to a blood plasma level of the Factor VIII protein in the absence of the antibody. In some embodiments, the accumulation of the Factor VIII protein results in a blood plasma level of the Factor VIII protein that is at least 5%, 10%, 20%, 30%0, 40%, 50%, 60%, 70%, 80%, 90%, 100%, 150%, 200%, 250%, 300%, 350%, 400%, 450%, 500%, 550%, 600%, 650%, 700%, 750%, 800%, 850%, 900%, 950%, 1,000%, 1,500%, 2,000%, 2,500%, 3,000%, 3,500%, 4,000%, 4,500%, 5,000%, 5,500%, 6,000%, 6,500%, 7,000%, 7,500%, 8,000%, 8,500%, 9,000%, 9,500%, 10,000%, or greater as compared to the blood plasma level of a VWF protein in the absence of the antibody. In some embodiments, the degree of accumulation of a FVIII protein depends on the effective amount of an antibody or antigen-binding fragment thereof administered to a subject, as described herein.

Also disclosed herein, in certain embodiments, are methods of increasing blood-plasma half-life of a VWF protein in a subject in need thereof, comprising administering to the subject a composition comprising an antibody or pharmaceutical composition disclosed herein. In some embodiments, the antibody increases the blood plasma half-life of the VWF protein by at least 1.5-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold as compared to the blood plasma half-life of the VWF protein in the absence of the antibody. In some embodiments, the antibody increases the blood plasma half-life of the VWF protein by at least 5%, 10%, 20%, 30%, 40%, 50%, 60%, 70%, 80%, 90%, 100%, 150%, 200%, 250%, 300%, 350%, 400%, 450%, 500%, 550%, 600%, 650%, 700%, 750%, 800%, 850%, 900%, 950%, 1,000%, 1,500%, 2,000%, 2,500%, 3,000%, 3,500%, 4,000%, 4,500%, 5,000%, 5,500%, 6,000%, 6,500%, 7,000%, 7,500%, 8,000%, 8,500%, 9,000%, 9,500%, or 10,000% as compared to the blood plasma half-life of the VWF protein in the absence of the antibody.

Any of the methods disclosed herein, in some instances, further include administering to the subject an additional therapeutic modality. Non-limiting examples of additional therapeutic modalities include therapeutic agents other than the antibody of the disclosure (e.g., compounds, peptides, nucleic acids, gene therapy, cell therapy etc.). In some embodiments, other antibodies, small molecule therapeutics, and/or other agents are combined in separate compositions for simultaneous or sequential administration. In one embodiment, simultaneous administration comprises one or more compositions that are administered at the same time, or within 30 minutes of each other. In some embodiments, administration occurs at the same or different sites.

Toxicity and therapeutic efficacy of such ingredient are, in some embodiments, determined by standard pharmaceutical procedures in cell cultures or experimental animals, e.g., for determining the LD₅₀ (the dose lethal to 50% of the population) and the ED₅₀ (the dose therapeutically effective in 50% of the population). In some embodiments, the dose ratio between toxic and therapeutic effects is the therapeutic index and is expressed as the ratio LD₅₀/ED₅₀. While compounds that exhibit toxic side effects are used in some embodiments, care should be taken to design a delivery system that targets such compounds to the site of affected tissue to minimize potential damage to healthy cells and, thereby, reduce side effects.

Data obtained from cell culture assays and animal studies are used in formulating a range of dosage for use in humans, in some embodiments. The dosage of such compounds lies preferably within a range of circulating concentrations that include the ED₅₀ with little or no toxicity. In some embodiments, the dosage varies within this range depending upon the dosage form employed and the route of administration utilized. In some embodiments, a dose is formulated in animal models to achieve a circulating plasma concentration arrange that includes the ED₅₀. Levels in plasma are measured, e.g., by high performance liquid chromatography, mass spectrometry, or other routine methods. Such information is, in some cases, used to more accurately determine useful doses in humans.

### Selection of Subjects

Subjects that may be treated as described herein are subjects having a disease or disorder, such as, e.g., a blood disorder characterized by excessive bleeding, as described herein. In some embodiments, the blood disorder is selected from the group consisting of von Willebrand disease (VWD), hemophilia A, disorders of platelet function, and connective tissue disorders. In some embodiments, the VWD is congenital (cVWD) or acquired (aVWD). In some embodiments, the aVWD includes Heydes syndrome. In some embodiments, the VWD is type 1, type 2A, type 2B, type 2M, type 2N, or type 3. In some embodiments, a subject selected for treatment with an antibody of the disclosure is a subject having VWD who has previously shown responsiveness to DDAVP (desmopressin). DDAVP increases plasma levels of VWF protein when administered in a therapeutically effective amount. In some embodiments, the subject has been identified (e.g., diagnosed) as having a blood disorder characterized by excessive bleeding. In some embodiments, the subject has a blood disorder characterized by excessive bleeding but has not been diagnosed as such.

### Route of Administration

Antibodies or antigen-binding fragments thereof are formulated for any suitable route of administration to a subject including, but not limited to injection (e.g., intravenous injection), in some embodiments. Injection includes, e.g., subcutaneous, peritoneal, intravenous injection, intramuscular injection, or spinal injection into the cerebrospinal fluid (CSF). In some embodiments, the antibodies of the disclosure are formulated for subcutaneous administration. In some embodiments, the antibodies of the disclosure are formulated for peritoneal administration. In some embodiments, the antibodies of the disclosure are formulated for intravenous administration (e.g., intravenous injection or infusion). In some embodiments, the antibodies of the disclosure are formulated for intramuscular administration. In some embodiments, the antibodies of the disclosure are formulated for spinal injection into the cerebrospinal fluid (CSF) administration. In some embodiments, administration is in one, two, three, four, five, six, seven, or more injection sites. In some embodiments, administration is in one injection site. In some embodiments, administration is in two injection sites. In some embodiments, administration is in three injection sites. In some embodiments, administration is in four injection sites. In some embodiments, administration is in five injection sites. In some embodiments, administration is in six injection sites.

For in vivo applications, contacting occurs, e.g., via administration of a composition (e.g., a composition described herein) to a subject by any suitable means. An antibody described herein, in some embodiments, is administered, e.g., either systemically or locally, including via parenteral, subcutaneous, intraperitoneal, intracerebrospinal, intrapulmonary, and intranasal administration, and, if desired for local treatment, intralesional administration. Parenteral routes include, e.g., intravenous, intraarterial, intraperitoneal, epidural, intramuscular, and intrathecal administration. Such administration, in some embodiments, is as a bolus, continuous infusion, or pulse infusion. In some embodiments, compositions are administered by injection depending in part on whether the administration is brief or chronic. Other modes of administration methods are contemplated, including topical, particularly transdermal, transmucosal, rectal, oral, or local administration e.g., through a catheter placed close to the desired site.

### Dosage

The compositions are administered in a manner compatible with the dosage formulation and in a therapeutically effective amount. The quantity to be administered depends on the subject to be treated and degree of binding capacity desired. Precise amounts of active ingredient required to be administered depend on the judgment of the practitioner and are peculiar to each subject. Suitable regimes for initial administration and booster shots are also variable, but are typified by an initial administration followed by repeated doses at one hour intervals or longer by a subsequent injection or other administration. Alternatively, continuous intravenous infusion that is sufficient to maintain concentrations in the blood are contemplated.

The amounts of the active ingredients (e.g., antibody of the disclosure) in the compositions, the composition formulation, and the mode of administration, are among the factors that are varied to provide an amount of the active ingredient that is effective to achieve the desired therapeutic response for each subject, without being unduly toxic to the subject. The selected dosage level will depend upon a variety of factors including the activity of the particular compound employed, the route of administration, the time of administration, the rate of excretion or metabolism of the particular compound being employed, the duration of the treatment, other drugs, compounds and/or materials used in combination with the particular composition employed, the age, sex, weight, condition, general health, diet and prior medical history of the subject being treated, and like factors well known in the medical arts.

In some embodiments, the antibodies described herein are administered to a subject in various dosing amounts and over various time frames. Additionally, the dose(s) of an antibody or antigen-binding fragment are administered, in some embodiments, twice a week, weekly, every two weeks, every three weeks, every 4 weeks, every 6 weeks, every 8 weeks, every 12 weeks, or any combination of weeks therein. Dosing cycles are also contemplated, such as, e.g., administering antibodies or antigen-binding fragments thereof once or twice a week for 4 weeks, followed by two weeks without therapy. Additional dosing cycles including, e.g., different combinations of the doses and weekly cycles described herein are also contemplated within the disclosure.

Therapeutically effective amounts of a composition, in some embodiments, vary and depend on the severity of the disease, the subject's weight, and general state of the subject being treated. Administration is, in some embodiments, daily, on alternating days, weekly, twice a month, monthly, or more or less frequently, as necessary depending on the response of the disorder or condition and the subject's tolerance to the therapy. In some embodiments, maintenance dosages over a longer period of time, such as 4, 5, 6, 7, 8, 10, or 12 weeks or longer, are needed until a desired suppression of disorder symptoms occurs, and dosages are adjusted as necessary. The progress of this therapy is easily monitored by conventional techniques and assays.

A physician or veterinarian having ordinary skill in the art, in some cases, readily determines and prescribes the effective amount (ED₅₀) of the composition required. For example, the physician or veterinarian could start doses of the compounds employed in the composition at levels lower than that required to achieve the desired therapeutic effect and gradually increase the dosage until the desired effect is achieved. Alternatively, a dose remains constant in some embodiments.

### Medicaments

The disclosure provides, in some embodiments, a use of the compositions described herein to make a medicament for treating a condition, disease, or disorder described herein. In some embodiments, medicaments are formulated based on the physical characteristics of the subject needing treatment and are formulated in single or multiple formulations based on the stage of the condition, disease, or disorder. Medicaments are packaged in a suitable package with appropriate labels for the distribution to hospitals and clinics in which the label is for the indication of treating a subject having a disease described herein in some embodiments. Medicaments are packaged as a single or multiple units in some embodiments. Instructions for the dosage and administration of the compositions are included with the packages as described below in some embodiments. The disclosure is further directed to medicaments comprising an antibody or antigen-binding fragment thereof described herein and a pharmaceutically acceptable carrier.

### Treatment Efficacy

A response is achieved when the subject experiences partial or total alleviation or reduction of signs or symptoms of illness. In some embodiments, an antibody or composition of the disclosure described herein is administered in an amount and for a time effective to result in reduction in one or more (e.g., 1 or more, 2 or more, 3 or more, 4 or more, 5 or more, 6 or more, 7 or more, 8 or more, 9 or more, or 10 or more) symptoms selected from the group consisting of epistaxis, cutaneous bleeding, bleeding from minor wounds, oral-cavity bleeding, angiodysplasia, gastrointestinal bleeding, bleeding from tooth extraction, postoperative bleeding, heavy menstrual bleeding, obstetric hemorrhage, hematuria, muscle hematoma, joint bleeding, visceral bleeding, and CNS bleeding. Reduction in the severity of the aforementioned symptoms is, in some embodiments, by any amount, so long as a therapeutic benefit is achieved in the patient. In some embodiments, a therapeutic response is achieved with the composition of the disclosure promotes platelet activation, adhesion, and/or aggregation in the subject. For example, platelet activation, adhesion, and or aggregation is, in some embodiments, increased in the subject following administration of a composition described herein by, e.g., 5%, 10%, 20%, 30%, 40%, 50%, 60%, 70%, 80%, 90%, 100%, 150%, 200%, 250%, 300%, 350%, 400%, 450%, 500%, 550%, 600%, 650%, 700%, 750%, 800%, 850%, 900%, 950%, 1,000%, 1,500%, 2,000%, 2,500%, 3,000%, 3,500%, 4,000%, 4,500%, 5,000%, 5,500%, 6,000%, 6,500%, 7,000%, 7,500%, 8,000%, 8,500%, 9,000%, 9,500%, 10,000%, or more as compared to the levels of platelet activation, adhesion, and aggregation in a control condition (e.g., in the absence of the composition). Platelet activation, adhesion, and/or aggregation is, in some embodiments, ascertained using methods well known in the art. Treatment efficacy is measured across different timeframes, in some embodiments, including, e.g., in months to years, depending on prognostic factors including the number of relapses, stage of disease, and other factors.

Prolonging survival is another desired treatment benchmark that, in some embodiments, includes, without limitation, increase in survival time by at least 1 month (mo), about at least 2 months (mos), about at least 3 mos, about at least 4 mos, about at least 6 mos, about at least 1, 2, 3, 4, 5, 6, 7, 8, 9, 10, 20, 30, 40, 50 years, or more. In some embodiments, overall survival is measured in months to years. In some embodiments, the subject's symptoms remain static or decrease.

In some embodiments, the methods described herein include identifying a patient as having a blood disorder characterized by excessive bleeding, as described herein. For example, an exemplary method includes: (a) identifying (e.g., diagnosing) a patient as having a blood disorder described herein; (b) optionally, evaluating the patient for levels of expression and/or activity of VWF or FVIII protein in blood plasma prior to treatment; and (c) administering to the patient a therapeutically effective dose of a disclosed composition, thereby treating the patient, in some embodiments. The method optionally includes, e.g., a further step (d) of assessing the patient for blood plasma levels of expression and/or activity of VWF or FVIII protein after administration of the composition to determine if the composition produced a therapeutically effective increase in the levels or activity of these proteins, in some embodiments. Assessment of levels of expression of activity of proteins is performed, in some embodiments, according to routine and conventional methods.

### Diagnostic Products and Methods

Disclosed herein, in certain embodiments, are methods of detection of a VWF protein in a sample or a subject to assess a treatment state of a patient or diagnose a disease or disorder described herein.

In the in vivo detection, diagnosis or monitoring of soluble VWF protein, or expression of a VWF protein by cells or tissues, a subject is administered an antibody or antigen-binding fragment as described herein, which antibody or antigen-binding fragment is bound to a detectable moiety. The detectable moiety is visualized, in some embodiments, using art-recognized methods such as, but not limited to, magnetic resonance imaging (MRI), fluorescence, radioimaging, light sources supplied by endoscopes, laparoscopes, or intravascular catheter (i.e., via detection of photoactive agents), photoscanning, positron emission tomography (PET) scanning, whole body nuclear magnetic resonance (NMR), radioscintigraphy, single photon emission computed tomography (SPECT), targeted near infrared region (NIR) scanning, X-ray, ultrasound. Labels for detecting compounds using such methods are also known in the art. Visualization of the detectable moiety allows, in some embodiments, for detection, diagnosis, and/or monitoring of a disease or disorder in the subject. Additional diagnostic assays that utilize antibodies specific to the desired target protein are known in the art and are also contemplated herein.

For in vitro detection methods, samples to be obtained from a subject include, but are not limited to, blood, tissue biopsy samples, and fluid therefrom.

Thus, the disclosure provides antibodies and antigen-binding fragments thereof that are useful for detecting or diagnosing VWF level or activity associated with one or more of the disorders described herein, potentially indicating need for therapeutic treatment. In some embodiments, the antibody further comprises a second agent. Such an agent, in some embodiments, is a molecule or moiety such as, e.g., a reporter molecule or a detectable label. Detectable labels/moieties for such detection methods are known in the art and are described in more detail below. Reporter molecules are any moiety which are detected using an assay. Non-limiting examples of reporter molecules which have been conjugated to polypeptides include enzymes, radiolabels, haptens, fluorescent labels, phosphorescent molecules, chemiluminescent molecules, chromophores, luminescent molecules, photoaffinity molecules, and colored particles or ligands, such as biotin. In some embodiments, detectable labels include compounds and/or elements that are detected due to their specific functional properties, and/or chemical characteristics, the use of which allows the polypeptide to which they are attached to be detected, and/or further quantified if desired. Many appropriate detectable (imaging) agents are known in the art, as are methods for their attachment to polypeptides.

Polypeptides are conjugated to a wide variety of fluorescent dyes, quenchers, and haptens such as fluorescein, R-phycoerythrin, and biotin in some embodiments. In some embodiments, conjugation occurs either during polypeptide synthesis or after the polypeptide has been synthesized and purified.

Alternatively, an antibody, antigen-binding fragment or binding protein is conjugated with a fluorescent moiety in some embodiments. Conjugating polypeptides with fluorescent moieties (e.g., R-Phycoerythrin, fluorescein isothiocyanate (FITC), etc.) is, e.g., accomplished using art-recognized techniques. Numerous commercially available fluorescent dyes and dye-conjugation kits are commercially available for particular applications, such as fluorescence microscopy, flow cytometry, fluorescence-activated cell sorting (FACS), etc.

In one non-limiting embodiment, an antibody antigen-binding fragment is associated with (conjugated to) a detectable label, such as a radionuclide, a dye, an imaging agent, or a fluorescent agent for immunodetection of binding to antigen which is used to visualize binding of the antibodies to soluble or bound VWF protein in vitro and/or in vivo.

Non-limiting examples of radiolabels include, e.g., ³²P, ³³P, ⁴³K, ⁵²Fe, ⁵⁷Co, ⁶⁴Cu, ⁶⁷Ga, ⁶⁷Cu, ⁶⁸Ga, ⁷¹Ge, ⁷⁵Br, ⁷⁶Br, ⁷⁷Br, ⁷⁷As, ⁷⁷Br, ⁸¹Rb/^{81m}Kr, ^{87m}Sr, ⁹⁰Y, ⁹⁷Ru, ⁹⁹Tc, ^{99m}Tc, ¹⁰⁰Pd, ¹⁰¹Rh, ¹⁰³Pb, ¹⁰⁵Rh, ¹⁰⁹Pd, ¹¹¹Ag, ¹¹¹In, ¹¹³In, ¹¹⁹Sb, ¹²¹Sn, ¹²³I, ¹²⁵I, ¹²⁷Cs, ¹²⁸Ba, ¹²⁹Cs, ¹³¹I, ¹³¹Cs, ¹⁴³Pr, ¹⁵³Sm, ¹⁶¹Tb, ¹⁶⁶Ho, ¹⁶⁹Eu, ¹⁷⁷Lu, ¹⁸⁶Re, ¹⁸⁸Re, ¹⁸⁹Re, ¹⁹¹Os, ¹⁹³Pt, ¹⁹⁴Ir, ¹⁹⁷Hg, ¹⁹⁹Au, ²⁰³Pb, ²¹¹At, ²¹²Pb, ²¹²Bi, and ²¹³Bi. In some embodiments, radiolabels are attached to compounds using conventional chemistry known in the art of antibody imaging. Radiolabeled compounds are useful in in vitro diagnostics techniques and in in vivo radioimaging techniques and in radioimmunotherapy.

Compositions of antibodies and antigen-binding fragments described herein are also used as non-therapeutic agents (e.g., as affinity purification agents) in some embodiments.

### Combination Therapy

The antibodies and compositions described herein are administered in combination with one or more (e.g., 1, 2, 3, 4, 5 or more) additional therapeutic agents or modalities for treatment of a disease or disorder described herein (e.g., a blood disorder characterized by excessive bleeding), in some embodiments.

In some embodiments, the one or more additional therapeutic agents is desmopressin (DDAVP), tranexamic acid (TXA), thalidomide, tamoxifen, ocreotide, a concentrate of plasma-derived VWF protein, a concentrate of VWF protein in complex with FVIII (VWF.FVIII; e.g., Humate P and Wilate), or a concentrate of recombinant VWF protein (e.g., Vonicog alfa), or a concentrate of FVIII protein (e.g., Alphanate and Koate-HP). In cases where the blood disorder is heavy menstrual bleeding, the one or more additional therapeutic agents is a hormonal treatment, such as, e.g., an oral contraceptive pill (e.g., an estrogen-containing contraceptive) or an intrauterine device, in some embodiments. In some embodiments, the one or more additional therapeutic modalities is gene therapy. In some embodiments, the gene therapy is FVIII gene therapy. For example, an antibody or composition of the disclosure may be administered in combination with a FVIII gene therapy to extend the half-life of FVIII and/or promote accumulation of FVIII in blood plasma of a subject. In some embodiments, an antibody or composition of the disclosure is administered to a patient for whom FVIII gene therapy alone is insufficient to attain therapeutically effective levels of FVIII in blood plasma. In some embodiments, an antibody or composition of the disclosure achieves therapeutically effective levels of FVIII in blood plasma.

In some embodiments, the one or more additional therapeutic modalities include blood transfusion, platelet transfusion, surgery, or cauterization. In some embodiments, the one or more additional therapeutic modalities include blood transfusion. In some embodiments, the one or more additional therapeutic modalities include platelet transfusion. In some embodiments, the one or more additional therapeutic modalities include surgery. In some embodiments, the one or more additional therapeutic modalities include cauterization.

In some embodiments, a composition (an antibody or an antigen-binding fragment described herein) is administered alone or in combination with a second composition either simultaneously or sequentially dependent upon the condition to be treated. When two or more compositions are administered, the compositions are, e.g., administered in combination (either sequentially or simultaneously). In some embodiments, the one or more additional therapeutic agents or modalities are administered immediately before or after the composition, or 5 minutes, 10 minutes, 15 minutes, 20 minutes, 25 minutes, 30 minutes, 45 minutes, 1 hour, 2 hours, 4 hours, 8 hours, 12 hours, 18 hours, 1 day, 2 days, 3 days, 4 days, one week, two weeks, 1 month, or more before or after administration of the composition. In some embodiments, a composition is administered in a single dose or multiple doses.

### Antibody Technology

As will be understood by the skilled artisan, general description of antibodies herein and methods of preparing and using the same also apply to individual antibody polypeptide constituents and antibody fragments.

The antibodies of the present disclosure are monoclonal or polyclonal antibodies. In some embodiments, the antibodies of the disclosure are monoclonal. In some embodiments, the antibodies of the disclosure are polyclonal. In particular embodiments, antibodies of the present disclosure are human antibodies. Methods of producing polyclonal and monoclonal antibodies are known in the art (see, e.g., Azevedo Reis Teixeira et al. mAbs 13:1 (2021). Antibodies, antigen-binding fragments, and other proteins that bind VWF are generated using such methods are tested for one or more of their binding affinity, avidity, and modulating capabilities in some embodiments.

In some embodiments, the antibodies of the present disclosure are one-armed antibodies (e.g., comprising a single heavy chain, a truncated heavy chain lacking a Fab region, and a single light chain). In some embodiments, the antibodies of the present disclosure are intact immunoglobulin molecules, such as, e.g., a human antibody, as well as those portions of a humanized Ig molecule that contain the antigen-binding site (i.e., paratope) or a single heavy chain and a single light chain, including those portions known in the art as Fab, Fab′, F(ab)′, F(ab′)2, Fd, scFv, a variable heavy domain, a variable light domain, a variable NAR domain, a single chain binding polypeptide, a dAb fragment, a nanobody, a VHH, and others also referred to as antigen-binding fragments. When constructing an immunoglobulin molecule or fragments thereof, variable regions or portions thereof are, in some embodiments, fused to, connected to, or otherwise joined to one or more constant regions or portions thereof to produce any of the antibodies or fragments thereof described herein. Thus, in some embodiments, the antigen-binding fragment of any one of the antibodies described above is a Fab, Fab′, F(ab)′, F(ab′)2, Fd, scFv, a variable heavy domain, a variable light domain, a variable NAR domain, a dAb fragment, a nanobody, a VHH, a single chain binding polypeptide (e.g., a scFv with Fc portion) or any other functional fragment thereof as described herein.

In some embodiments, antibodies of the present disclosure are of any immunoglobulin class, and, therefore, in some embodiments, have a gamma, mu, alpha, delta, or epsilon heavy chain. In some embodiments, the gamma chain is gamma 1, gamma 2, gamma 3, or gamma 4. In some embodiments, the alpha chain is alpha 1 or alpha 2.

In some embodiments, an antibody of the present disclosure is an IgA immunoglobulin. In some embodiments, antibodies of the present disclosure are of any IgA subclass. In some embodiments, the antibody is IgA1. In some embodiments, the antibody is IgA2. In some embodiments, an antibody of the present disclosure is an IgD immunoglobulin. In some embodiments, an antibody of the present disclosure is an IgE immunoglobulin. In some embodiments, an antibody of the present disclosure is an IgG immunoglobulin. In some embodiments, antibodies of the present disclosure are of any IgG subclass. In some embodiments, the antibody is IgG1. In some embodiments, the antibody is IgG2. In some embodiments, the antibody is IgG3. In some embodiments, the antibody is IgG4. In some embodiments, an antibody of the present disclosure is an IgM immunoglobulin.

In some embodiments, antibodies of the present disclosure comprise a variable light chain that is either kappa or lambda. In some embodiments, the lambda chain is of any subtype, including, e.g., lambda 1, lambda 2, lambda 3, and lambda 4. In some embodiments, the light chain is kappa.

In some embodiments, antibodies disclosed herein comprise a human variable framework region and a human constant region. In some embodiments, the antibodies comprise a human light chain variable framework region and a human light chain constant region. In some embodiments, the antibodies comprise a human heavy chain variable framework region and a human heavy chain constant region. In some embodiments, the antibodies comprise a human light chain variable framework region, a human light chain constant region, a human heavy chain variable framework region, and a human heavy chain constant region.

In some embodiments, an antibody of the present disclosure comprises a human variable framework region and a murine constant region. In some embodiments, an antibody of the present disclosure comprises a human heavy chain variable framework region and a murine heavy chain constant region. In some embodiments, an antibody of the present disclosure comprises a human light chain variable framework region, a murine light chain constant region, a human heavy chain variable framework region, and a murine heavy chain constant region.

In some embodiments, an antibody of the present disclosure has one binding site for the VWF protein (e.g., a binding site to a single epitope of the VWF protein). In some embodiments, an antibody of the present disclosure is monospecific. Exemplary monospecific antibodies bind one epitope of a single antigen. In some embodiments, the monospecific antibody is a monovalent antibody (e.g., having a single Fab domain and a single Fc domain; a one-armed antibody).

In some embodiments, an antibody of the present disclosure is bispecific or multi-specific. Bispecific antibodies are antibodies that have binding specificities for at least two different epitopes. Exemplary bispecific antibodies, in some embodiments, bind to two different epitopes of a single antigen. Other such antibodies, in some embodiments, combine a first antigen binding site with a binding site for a second antigen. In some embodiments, the bispecific antibodies bind at least two different epitopes and have constant domains that bind to Fc receptors. In some embodiments, the binding of one or more epitopes of the bispecific antibodies is simultaneous with binding of the constant domains of the bispecific antibodies to Fc receptors.

In some embodiments, an antibody of the present disclosure has two or more valences, which are also referred to as multivalent. In some embodiments, an antibody of the present disclosure is trispecific. In some embodiments, the multivalent antibody is internalized (and/or catabolized) faster than a bivalent antibody by a cell expressing an antigen to which the antibodies bind. In some embodiments, the antibodies of the present disclosure are multivalent antibodies with three or more antigen binding sites (e.g., tetravalent antibodies). In some embodiments, the multivalent antibodies of the present disclosure are produced by recombinant expression of nucleic acid encoding the polypeptide chains of the antibody. In some embodiments, the multivalent antibody comprises a dimerization domain and three or more antigen binding sites. In some embodiments, the dimerization domain comprises (or consists of) an Fc region or a hinge region. In this scenario, the antibody will comprise an Fc region and three or more antigen binding sites amino-terminal to the Fc region. In some embodiments, the multivalent antibody herein comprises about three to about eight, but preferably four, antigen binding sites. The multivalent antibody comprises at least one polypeptide chain (and preferably two polypeptide chains), wherein the polypeptide chain(s) comprise two or more variable regions. For instance, the polypeptide chain(s) comprises VD1-(X1)ₙ-VD2-(X2)ₙ-Fc, wherein VD1 is a first variable region, VD2 is a second variable region, Fc is one polypeptide chain of an Fc region, X1 and X2 represent an amino acid or polypeptide, and n is 0 or 1. In some embodiments, the polypeptide chain(s) each independently comprise: V_{H}-C_{H}1-flexible linker-V_{H}-C_{H}1-Fc region chain; or V_{H}-C_{H}1-V_{H}-C_{H}1-Fc region chain. In some embodiments, the multivalent antibody herein further comprises at least two (and preferably four) light chain variable region polypeptides. In some embodiments, the multivalent antibody herein comprises from about two to about eight light chain variable region polypeptides. In some embodiments, the light chain variable region polypeptides described herein comprise a light chain variable region. In some embodiments, the light chain variable region polypeptides described herein further comprise a C_{L} domain.

In some embodiments, an antibody of the present disclosure is constructed to fold into multivalent forms, which, in some embodiments, improves binding affinity, specificity, and/or increased half-life in blood. Multivalent forms of antibodies are prepared, for example, by techniques known in the art.

In some embodiments, an antibody of the present disclosure is an SMIP or binding domain immunoglobulin fusion protein specific for the target protein. These constructs are single-chain polypeptides comprising antigen-binding domains fused to immunoglobulin domains necessary to carry out antibody effector functions.

In some embodiments, an antibody of the present disclosure comprises a single chain binding polypeptide having a heavy chain variable region, and/or a light chain variable region which binds an epitope disclosed herein and has, optionally, an immunoglobulin Fc region. Such a molecule is a single chain variable fragment (scFv) optionally having effector function or increased half-life through the presence of the immunoglobulin Fc region

In some embodiments, VWF-binding antibodies include at least one heavy chain and at least one light chain. In some embodiments, VWF-binding antibodies include one heavy chain and one light chain. In some embodiments, VWF-binding antibodies include at least one heavy chain comprising a heavy chain variable domain (V_{H}) and at least one light chain comprising a light chain variable domain (V_{L}). In some embodiments, the VWF-binding antibodies of the disclosure include a single full-length heavy chain, a single truncated heavy chain lacking V_{H} and CH1 domains, and a single light chain. In some embodiments, VWF antibodies include one heavy chain comprising a V_{H} and one light chain comprising a V_{L}. Each V_{H} and V_{L} comprises three complementarity determining regions (CDRs). The amino acid sequences of the V_{H} and V_{L} and the CDRs determine the antigen binding specificity and antigen binding strength of the antibody.

In some embodiments, the antibody disclosed herein is a monoclonal antibody. In some embodiments, an antibody disclosed herein is a monospecific and monovalent antibody containing a single arm of an immunoglobulin molecule (e.g., a one-armed antibody that includes a single full-length heavy chain, a single truncated heavy chain lacking V_{H} and CH1 domains, and a single light chain). In some embodiments, an antibody disclosed herein is selected from a half-immunoglobulin (e.g., a one-armed antibody) or whole immunoglobulin. In some embodiments, an antibody disclosed herein is a one-armed antibody. In some embodiments, an antibody disclosed herein is an antigen binding fragment. In some embodiments, an antibody disclosed herein is human. In some embodiments, an antibody disclosed herein is humanized. In some embodiments, an antibody disclosed herein is chimeric.

In some embodiments, conventional methods are utilized to identify antibodies or antigen-binding fragments thereof that bind to a VWF protein (e.g., a human or NHP VWF protein). In some embodiments, antibodies and antigen-binding fragments are evaluated for one or more of binding affinity, association rates, disassociation rates, and avidity. Measurement of such parameters is, e.g., accomplished using assays including, but not limited to, an enzyme-linked-immunosorbent assays (ELISA), ELISpot assays, Scatchard analysis, surface plasmon resonance (e.g., BIACORE) analysis, etc., competitive binding assays, and the like. In a non-limiting embodiment, an ELISA assay is used to measure the binding capability of specific antibodies or antigen-binding fragments that bind to a VWF protein.

In some embodiments, antibodies according to the disclosure are produced recombinantly, using vectors and methods available in the art, as described further below.

In some embodiments, human antibodies are produced in transgenic animals (e.g., mice) that are capable of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array into such germ-line mutant mice results in the production of human antibodies upon antigen challenge. In some embodiments, such animals are genetically engineered to produce human antibodies comprising a polypeptide of the present disclosure.

The antibodies are, e.g., isolated and purified from a culture supernatant or ascites (if produced in an animal) using methods known in the art, such as by saturated ammonium sulfate precipitation, euglobulin precipitation method, caproic acid method, caprylic acid method, ion exchange chromatography (DEAE or DE52), or affinity chromatography using anti-Ig column or a protein A, G, or L column.

As noted above, the disclosure further provides antibody fragments. In certain circumstances, there are advantages of using antibody fragments, rather than whole antibodies. For example, the smaller size of the fragments allows for rapid clearance, and leads to improved access to certain tissues, such as organs (e.g., lung, kidney, liver, or heart). Examples of antibody fragments include: Fab, F(ab′), F(ab′)₂, and Fv fragments, diabodies, linear antibodies, single-chain antibodies, and multispecific antibodies formed from antibody fragments.

Various techniques have been developed to produce antibody fragments. Traditionally, these fragments were derived via proteolytic digestion of intact antibodies. However, these fragments can be produced directly by recombinant host cells. Fab, Fv, and scFv antibody fragments may also be expressed in and secreted from E. coli, thus allowing the facile production of large amounts of these fragments. F(ab′)-SH fragments can be directly recovered from E. coli and chemically coupled to form F(ab′)₂ fragments. Fab fragment with increased in vivo half-life comprising a salvage receptor binding epitope taken from two loops of a C_{H}2 domain of an Fc region of an IgG have also been designed. Other techniques for producing antibody fragments will be apparent to the skilled practitioner.

The antibody of choice can be a single chain Fv fragment (scFv). Fv and sFv are the only species with intact combining sites that are devoid of constant regions. Thus, they are suitable for reduced nonspecific binding during in vivo use. sFv fusion proteins can be constructed to yield fusion of an effector protein at either the amino or the carboxy terminus of an sFv. The antibody fragment can also be a “linear antibody.” In some embodiments, such linear antibody fragments are monospecific or bispecific.

Methods for making bispecific or other multispecific antibodies are known in the art and include chemical cross-linking, use of leucine zippers (diabody technology; scFv dimers, linear antibodies, and chelating recombinant antibodies.

Traditional production of full-length bispecific antibodies is based on the co-expression of two immunoglobulin heavy chain-light chain pairs, where the two chains have different specificities. Because of the random assortment of immunoglobulin heavy and light chains, these hybridomas (quadromas) produce a potential mixture of ten different antibody molecules, of which only one has the correct bispecific structure. Purification of the correct molecule can be done, for example, by affinity chromatography.

According to a different approach, antibody variable regions with the desired binding specificities (antibody-antigen combining sites) can be fused to immunoglobulin constant domain sequences. Preferably, the fusion is with an Ig heavy chain constant domain, comprising at least part of the hinge, C_{H}2, and C_{H}3 regions. It is preferred that the first heavy-chain constant region (C_{H}1) containing the site necessary for light chain bonding, be present in at least one of the fusions. DNAs encoding the immunoglobulin heavy chain fusions and, if desired, the immunoglobulin light chain, can be inserted into separate expression vectors, and are co-transfected into a suitable host cell. This provides for greater flexibility in adjusting the mutual proportions of the four polypeptide fragments in embodiments when unequal ratios of the four polypeptide chains used in the construction provide the optimum yield of the desired bispecific antibody. It is, however, possible to insert the coding sequences for two or all four polypeptide chains into a single expression vector when the expression of at least two polypeptide chains in equal ratios results in high yields or when the ratios have no significant effect on the yield of the desired chain combination.

Bispecific antibodies are composed of, for example, a hybrid immunoglobulin heavy chain with a first binding specificity in one arm, and a hybrid immunoglobulin heavy chain-light chain pair (providing a second binding specificity) in the other arm. This asymmetric structure facilitates the separation of the desired bispecific compound from unwanted immunoglobulin chain combinations, as the presence of an immunoglobulin light chain in only one half of the bispecific molecule provides for a facile way of separation.

In certain methods, the interface between a pair of antibody molecules can be engineered to maximize the percentage of heterodimers that are recovered from recombinant cell culture in some embodiments. The preferred interface comprises at least a part of the C_{H}3 domain. In this method, one or more small amino acid side chains from the interface of the first antibody molecule are replaced with larger side chains (e.g., tyrosine or tryptophan). Compensatory “cavities” of identical or similar size to the large side chain(s) can be created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells, and for treatment of HIV infection. In some embodiments, heteroconjugate antibodies can be made using any convenient cross-linking methods. Suitable cross-linking agents are well known in the art, along with a number of cross-linking techniques. Another method is designed to make tetramers by adding a streptavidin-coding sequence at the C-terminus of the scFv. Streptavidin is composed of four subunits, so when the scFv-streptavidin is folded, four subunits associate to form a tetramer.

According to another approach for making bispecific antibodies, the interface between a pair of antibody molecules are engineered to maximize the percentage of heterodimers which are recovered from recombinant cell culture in some embodiments. One interface comprises at least a part of the C_{H}3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Techniques for generating bispecific antibodies from antibody fragments have also been described in the literature. For example, bispecific antibodies are prepared using chemical linkage. An exemplary procedure includes proteolytically cleaving intact antibodies to generate F(ab′)₂ fragments. These fragments are reduced in the presence of the dithiol complexing agent, sodium arsenite, to stabilize vicinal dithiols, and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. In some embodiments, the bispecific antibodies produced are used as agents for the selective immobilization of enzymes.

Recent progress has facilitated the direct recovery of Fab′-SH fragments from E. coli, which are, for example, chemically coupled to form bispecific antibodies. An exemplary method of production includes separately secreting each Fab′ fragment from E. coli and subjecting the fragment to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can be used to produce antibody homodimers in some embodiments.

### Binding Affinity and Immunoreactivity

Binding affinity and/or avidity of antibodies or antigen-binding fragments thereof are improved by modifying framework regions. Any suitable methods for modifications of framework regions are known in the art and are contemplated herein. Selection of one or more relevant framework amino acid positions to alter depends on a variety of criteria. One criterion for selecting relevant framework amino acids to change is, e.g., the relative differences in amino acid framework residues between the donor and acceptor molecules. Selection of relevant framework positions to alter using this approach has the advantage of avoiding any subjective bias in residue determination or any bias in CDR binding affinity contribution by the residue.

Binding interactions are manifested as an intermolecular contact with one or more amino acid residues of one or more CDRs in some embodiments. Antigen-binding involves, e.g., a CDR or a CDR pair or, in some cases, interactions of up to all six CDRs of the VH and VL chains.

Binding affinity and avidity of antibodies or antigen-binding fragments is, in some embodiments, measured by surface plasmon resonance (SPR) measurements, ELISA, AlphaLisa assays, flow cytometry, and other conventional methods.

Disclosed herein are antibodies that specifically bind to a VWF protein with a K_{d} from 0.1 nM to 50 nM. In some embodiments, the antibodies specifically bind to a VWF protein with a K_{d} from about 0.1 to about 50 nM, from about 0.1 to about 20 nM, from about 0.1 to about 10 nM, from about 0.1 to about 5 nM, from about 0.1 to about 2 nM, from about 0.1 to about 1 nM, from about 0.1 to about 0.5 nM, from about 0.5 to about 50 nM, from about 0.5 to about 20 nM, from about 0.5 to about 10 nM, from about 0.5 to about 5 nM, from about 0.5 to about 2 nM, from about 0.5 to about 1 nM, from about 1 to about 50 nM, from about 1 to about 20 nM, from about 1 to about 10 nM, from about 1 to about 5 nM, from about 1 to about 2 nM, from about 2 to about 50 nM, from about 2 to about 20 nM, from about 2 to about 10 nM, from about 2 to about 5 nM, from about 5 to about 50 nM, from about 5 to about 20 nM, from about 5 to about 10 nM, from about 10 to about 50 nM, from about 10 to about 20 nM, or from about 20 to about 50 nM. In some embodiments, the disclosed antibodies bind to VWF with a K_{d} of less than 100 nM, 90 nM, 80 nM, 70 nM, 60 nM, 50 nM, 40 nM, 30 nM, 20 nM, 10 nM, 7 nM, 5 nM, 4 nM, 3 nM, 2 nM, 1 nM, 0.5 nM, 0.25 nM, or less. In some embodiments, the disclosed antibodies bind to VWF with a K_{d} of 0.1 nM to 10 nM (e.g., 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10 nM). In some embodiments, the disclosed antibodies bind to VWF with a K_{d} of 0.1 nM, 0.075 nM, 0.05 nM, 0.025 nM, 0.01 nM, or less.

### Identification and Preparation of Antibodies

Polynucleotide sequences encoding the antibodies, variable regions thereof, or antigen-binding fragments thereof are, in some embodiments, determined using conventional sequencing techniques, and subcloned into expression vectors for the recombinant production of the antibodies. Polynucleotides that encode the antibodies or portions thereof of the present disclosure are isolated from cells expressing the antibodies, according to methods available in the art and described herein, including amplification by polymerase chain reaction using primers specific for conserved regions of human antibody polypeptides, in some embodiments.

Polynucleotides encoding antibody chains, variable regions thereof, or fragments thereof, are isolated from cells utilizing any means available in the art in some embodiments. In one embodiment, polynucleotides are isolated using polymerase chain reaction (PCR), e.g., reverse transcription-PCR (RT-PCR) using oligonucleotide primers that specifically bind to heavy or light chain encoding polynucleotide sequences or complements thereof using routine procedures available in the art. In one embodiment, positive wells are subjected to whole-well RT-PCR to amplify the heavy and light chain variable regions of the immunoglobulin molecule expressed by the clonal daughter plasma cells. These PCR products, in some embodiments, are sequenced, and products encoding the heavy and light chain variable regions or portions thereof are then subcloned into human antibody expression vectors and recombinantly expressed according to routine procedures in the art. The nucleic acid molecules the antibody or fragment thereof as described herein are, in some embodiments, propagated and expressed according to any of a variety of well-known procedures for nucleic acid excision, ligation, transformation, and transfection. Thus, in certain embodiments, expression of an antibody fragment is preferred in a prokaryotic host cell, such as E. coli. In some embodiments, expression of the antibody or an antigen-binding fragment thereof is preferred in a eukaryotic host cell, such as yeast (e.g., Saccharomyces cerevisiae, S. pombe, Pichia pastoris); animal cells (including mammalian cells); or plant cells. Examples of suitable animal cells include, but are not limited to, myeloma, COS, CHO, or hybridoma cells. Examples of plant cells include tobacco, corn, soybean, and rice cells. By methods known to those having ordinary skill in the art and based on the present disclosure, a nucleic acid vector is designed for expressing foreign sequences in a particular host system, and then polynucleotide sequences encoding the antibody (or fragment thereof) is inserted, in some embodiments. The regulatory elements will vary according to the particular host.

One or more replicable expression vectors containing a polynucleotide encoding a variable and/or constant region is, in some embodiments, prepared and used to transform an appropriate cell line, e.g., a non-producing cell line, such as a mouse cell line or a bacterium, such as E. coli, in which production of the antibody will occur. In order to obtain efficient transcription and translation, the polynucleotide sequence in each vector should include appropriate regulatory sequences, particularly a promoter and leader sequence operatively linked to the variable region sequence.

Particular methods for producing antibodies in this way are generally well known and routinely used. For example, molecular biology procedures are described by Sambrook et al., Molecular Cloning, A Laboratory Manual, 2nd ed., Cold Spring Harbor Laboratory, New York, 1989; see also Sambrook et al., 3rd ed., Cold Spring Harbor Laboratory, New York, (2001)). While not required, in certain embodiments, regions of polynucleotides encoding the recombinant antibodies are sequenced. DNA sequencing are performed, e.g., in any manner or using any systems known in the art. Basic sequencing technology is described e.g., in Sanger et al., Proc Natl Acad Sci USA 74:5463 (1977)) and the Amersham International plc sequencing handbook and including improvements thereto. In some embodiments, the resulting recombinant antibodies or fragments thereof are then tested to confirm their original specificity, and are further tested for cross-reactivity, e.g., with related polypeptides. In particular embodiments, an antibody identified or produced according to methods described herein is tested for ability to perform a specified function (e.g., accumulation of VWF and/or FVIII in blood plasma, VWF half-life extension, etc.) using conventional methods.

### Packages, Kits, and Pre-Filled Containers

Also provided herein are kits containing one or more compounds described above. The kit comprises, in some embodiments, an antibody or antigen-binding fragment thereof as described herein in suitable container means.

In some embodiments, there is provided is a container means comprising a composition described herein. In some embodiments, the container means is any suitable container which houses, e.g., a liquid or lyophilized composition including, but not limited to, a vial, syringe, bottle, and an intravenous (IV) bag or ampoule. A syringe holds any volume of liquid suitable for injection into a subject, including, but not limited to, 0.5 cc, 1 cc, 2 cc, 5 cc, 10 cc, or more.

Provided herein are kits comprising a composition or compositions described herein. In some embodiments, provided herein is a kit for treating a subject having a blood disorder characterized by excessive bleeding comprising an antibody as described herein and, optionally, an additional therapeutic agent.

In some embodiments, provided herein is a kit for treating a blood disorder characterized by excessive bleeding comprising an antibody as described herein and a label attached to or packaged with the container, the label describing use of the antibody, optionally, in combination with an additional therapeutic agent.

In some embodiments, the container means of the kits will generally include at least one vial, test tube, flask, bottle, ampoule, syringe, an intravenous (IV) bag, and/or other container means, into which at least one antibody of the disclosure is placed, and/or preferably, suitably aliquoted. Provided herein is a container means comprising a composition described herein.

The kits, in some embodiments, include a means for containing at least one fusion protein, detectable moiety, reporter molecule, and/or any other reagent containers in close confinement for commercial sale. In some embodiments, such containers include injection and/or blow-molded plastic containers into which the desired vials are retained. In some embodiments, kits also include printed material for use of the materials in the kit.

Packages and kits additionally include a buffering agent, a preservative, and/or a stabilizing agent in a pharmaceutical formulation, in some embodiments. In some embodiments, each component of the kit is enclosed within an individual container and all of the various containers are within a single package. In some embodiments, disclosure kits are designed for cold storage or room temperature storage.

Additionally, in some embodiments, the preparations contain stabilizers to increase the shelf-life of the kits and include, e.g., bovine serum albumin (BSA). Where the compositions are lyophilized, the kit contains, in some embodiments, further preparations of solutions to reconstitute the lyophilized preparations. Acceptable reconstitution solutions are well known in the art and include, e.g., pharmaceutically acceptable phosphate buffered saline (PBS).

In some embodiments, packages and kits further include one or more components for an assay, such as, e.g., an ELISA assay. Samples to be tested in this application include, e.g., blood, plasma, serum, tissue sections and secretions, urine, lymph, and products thereof. In some embodiments, packages and kits further include one or more components for collection of a sample (e.g., a syringe, a cup, a swab, etc.).

In some embodiments, packages and kits further include a label specifying information required by US FDA or similar regulatory authority, e.g., a product description, amount and mode of administration, and/or indication of treatment. In some embodiments, packages provided herein include any of the compositions as described herein.

The term “packaging material” refers to a physical structure housing the components of the kit. In some embodiments, the packaging material maintains the components sterile and is made of material commonly used for such purposes (e.g., paper, corrugated fiber, glass, plastic, foil, ampules, etc.). In some embodiments, the label or packaging insert includes appropriate written instructions (e.g., instructing the user of the kit to perform one or more methods disclosed herein). Kits, in some embodiments, additionally include labels or instructions for using the kit components in any method of the disclosure. In some embodiments, a kit includes a compound in a pack or dispenser together with instructions for administering the compound in a method described herein.

In still further embodiments, a kit further comprises a container means for one or more additional therapeutics for a blood disorder characterized by excessive bleeding.

In some embodiments, instructions include instructions for practicing any of the methods described herein including treatment methods. In some embodiments, instructions additionally include indications of a satisfactory clinical endpoint or any adverse symptoms that occur, or additional information required by regulatory agencies such as the Food and Drug Administration for use on a human subject.

The instructions are, in some embodiments, on “printed matter,” e.g., on paper or cardboard within or affixed to the kit, or on a label affixed to the kit or packaging material, or attached to a vial or tube containing a component of the kit. Instructions are additionally included on a computer readable medium, such as, e.g., CD-ROMs, DVDs, flash memory devices, solid state memory, magnetic disks and disk devices, magnetic tapes, cloud computing systems and services, and the like, in some embodiments. In some cases, the program and instructions are permanently, substantially permanently, semi-permanently, or non-transitorily encoded on the media.

Provided herein is a container means comprising a composition described herein. In some embodiments, the container means is any suitable container which houses a liquid or lyophilized composition including, but not limited to a vial, syringe, bottle, intravenous (IV) bag, or ampoule. A syringe, in some embodiments, holds any volume of liquid suitable for injection into a subject, including but not limited to 0.5 cc, 1 cc, 2 cc, 5 cc, 10 cc or more.

Provided herein are kits comprising a composition described herein. In some embodiments, provided herein is a kit for treating a blood disorder characterized by excessive bleeding, comprising an antibody as described herein, optionally, in combination with one or more additional therapeutic agents.

In some embodiments, provided herein is a kit for treating a blood disorder characterized by excessive bleeding, comprising an antibody as described herein, and a label attached to or packaged with the container, the label describing use of the antibody or an antigen-binding fragment thereof as described herein, optionally, with an additional therapy. In some embodiments, the blood disorder characterized by excessive bleeding is VWD, hemophilia A, disorders of platelet function, and connective tissue disorders. In some embodiments, the VWD is cVWD or aVWD. In some embodiments, the aVWD includes Heydes syndrome. In some embodiments, the VWD is type 1, type 2A, type 2B, type 2M, type 2N, or type 3.

### EXAMPLES

The following examples are put forth to provide those of ordinary skill in the art with a description of how the compositions and methods described herein may be used, made, and evaluated, and are intended to be purely exemplary of the disclosure and are not intended to limit the scope of what the inventors regard as their invention.

### Example 1: Recombinant Expression and Purification of Von Willebrand Factor Cysteine Knot Domain Constructs

Protein constructs for the expression of the human and cynomolgus monkey (“cyno”) VWF cysteine knot (CK) domains were designed according to Zhou et al. Blood 123:1785-93 (2014) with the sequences provided in SEQ ID NO: 5 and SEQ ID NO: 6, respectively. In addition to the CK domain sequence, the constructs were equipped with an N-terminal signal peptide derived from VWF followed by a hexa-histidine tag (SEQ ID NO: 11), in vitro biotinylation tag, HRV-3C protease cleavage site, and 3 residues from VWF immediately prior to the CK domain. Expression, purification, and in vitro biotinylation were performed by Cambridge ProteinWorks (Cambridge, UK) using HEK293 EBNA cells (ThermoFisher Scientific) and the BirA biotin-protein ligase kit from Avidity (Colorado, USA), as per the manufacturer's recommendations. Consistent with the CK domain carrying one N-glycan and forming disulfide-linked homodimers (Zhou et al. Blood 123:1785-93 (2014)), the two protein constructs were found to migrate with apparent molecular weights of 35-kDa and 16-kDa under non-reducing and reducing conditions, respectively.

### Example 2. Generation of Anti-VWF CK Domain Antibodies Using Phage and Yeast Display

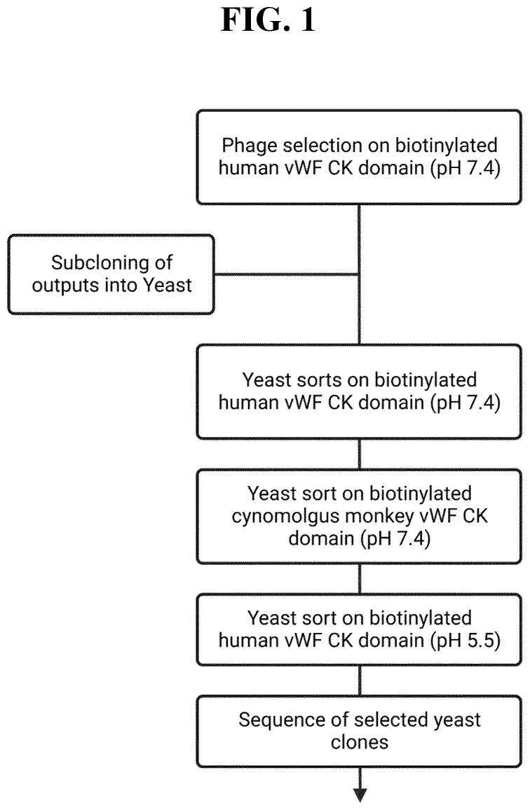

Human monoclonal antibodies to the human biotinylated VWF cysteine knot (CK)-domain were identified using the human Generation 3 library from Specifica Inc. (Santa Fe, NM, USA). Methods for antibody library design and antibody selections by phage and yeast display are well-known. During antibody selections, the biotinylated human VWF CK domain was used as a primary antigen. From the pool of apparent high-affinity antibody binders, antibodies cross-reacting with the biotinylated cyno VWF CK domain were selected. An overview of the selection protocol is provided in FIG. 1.

A total of 63 antibodies in single-chain variable fragment (scFv) format were identified from the phage/yeast selection campaign. Amino acid sequences of the variable heavy chain (VH) and variable light chain (VL) regions of each antibody are provided in Table 1 along with sequences of the associated complementarity determining region 1 (CDR1), CDR2, and CDR3 loops. CDR loop sequences were extracted from each VH and VL using ANARCI and the IMGT numbering scheme.

### Example 3: Production of Anti-VWF Antibodies Using Hybridoma Technology

Protein constructs containing a CK domain of VWF (e.g., SEQ ID NO: 5 and/or SEQ ID NO: 6) are injected (e.g., intravenously) into a mouse host by one or more (e.g., 1, 2, 3, 4, 5, 6, or more) injections over the course of several weeks. B cells clones are harvested from the mouse. The harvested B cells are fused with immortal myeloma cells using, e.g., electrofusion or polyethylene glycol (PEG) induction to produce hybridoma cells expressing antibodies specific to the CK domain of VWF (e.g., SEQ ID NO: 5 and/or SEQ ID NO: 6). Prior to fusion, myeloma cells are selected for ability to grow in culture and to ensure they do not intrinsically produce antibodies and lack expression of the HGPRT gene. Fused cells are cultured in HAT (hypoxanthine-aminoptern-thymidine) medium for about 10-14 days. Unfused myeloma cells are removed from culture. The incubated medium is diluted into multi-well plates, such that each well contains one hybridoma cell.

Following incubation, hybridomas are screened and selected for antibodies having desirable specificity to the CK domain of VWF. Screening is performed using standard techniques, such as, e.g., ELISA, western blot, immunocytochemistry, or immunoprecipitation-mass spectrometry. Selected hybridomas are transferred to tissue culture flasks and cultured to establish a colony of hybridoma cells that produce the anti-VWF antibody. Antibodies are isolated and purified using routine and conventional methods.

### Example 4: Production of Anti-VWF Antibodies Using Transgenic Animals

A transgenic animal host (e.g., mouse) is produced by genetically modifying the host germ line to replace endogenous loci encoding immunoglobulin (Ig) genes with a transgene(s) encoding human or non-human primate (e.g., Macaca fascicularis) Ig in the host genome. For example, the murine endogenous locus encoding variable, diversity, joining, and constant region gene segments is substituted with corresponding human gene segments as described in Jakobovits et al., Proc Natl Acad Sci USA, 90:2551 (1993); Jakobovits et al., Nature 362:255-58 (1993); Bruggemann et al., Year in Immunol., 7:33 (1993); U.S. Pat. Nos. 5,545,806, 5,569,825, 5,591,669; 5,545,807; and WO 97/17852. The transgenic host is subsequently inoculated with an antigen encoding the human (SEQ ID NO: 5) or non-human primate (SEQ ID NO: 6) CK domain of VWF protein to stimulate production of human or non-human primate antibodies against the CK domain. Optionally, B cells are isolated from the host and hybridoma cells are produced according to the methods described in Example 3. Antibodies targeting the CK domain of VWF are isolated and purified according to routine and conventional methods.

### Example 5: High-Throughput Screening of Identified Antibodies for Binding to Human and Non-Human Primate VWF CK Domain

The 63 identified antibody sequences from the phage/yeast selection campaign (FIG. 1; Examples 1 and 2) were cloned into a yeast expression vector (pFLYS6) and expressed in Saccharomyces cerevisiae as soluble scFvs equipped with C-terminal 3×FLAG and SV5 tags. The scFv constructs had a VH-linker-VL configuration with the linker consisting of the amino acid sequence SGGSTITSYNVYYTKLSSSGT (SEQ ID NO: 483).

To initially characterize the ability of the scFv constructs to bind biotinylated human and cyno VWF CK domain constructs, Surface Plasmon Resonance (SPR) experiments were conducted. Briefly, a sensor chip was activated and coupled with an anti-SV5 mouse monoclonal antibody using amine coupling. The soluble scFvs in crude yeast supernatants were printed on the anti-SV5 lawn. Binding to the biotinylated human VWF CK domain was measured at room temperature and pH 7.4 (in 10 mM HEPES, 150 mM NaCl, 3 mM EDTA, 0.05% Tween 20, pH 7.4 and 0.5 mg/mL BSA) or pH 5.5 (in 20 mM MES, 150 mM NaCl, pH 5.5, and 0.5 mg/mL BSA) by injection of a concentration range of CK domain from 0.3 to 500 nM. Similarly, binding to the biotinylated cyno VWF CK domain was measured at room temperature and pH 7.4. From the measured association (kₒₙ) and dissociation (k_{off}) rate constants, an apparent equilibrium dissociation constant (K_{d}=k_{off}/kₒₙ) was estimated. Due to the dimeric state of the CK construct and consequent possibility for simultaneous binding of captured scFv to both monomers in the CK dimer, the SPR setup was expected to overestimate the binding affinity. Nevertheless, high-throughput binding study demonstrated that all 63 scFv candidates were capable of binding the human VWF CK domain construct at pH 7.4 with apparent K_{d} values ranging from about 50 pM to 10 nM. Several scFv candidates retained binding to human VWF CK at pH 5.5 and cyno VWF CK at pH 7.4. Results are summarized in Table 2.

TABLE 2
Overview of binding properties of_scFv anti-VWF CK domain antibodies

| | Binding | Binding | Binding |
|---|---|---|---|
| | to human | to human | to cyno |
| | VWF CK | VWF CK | VWF CK |
| | at pH 7.4 | at pH 5.5 | at pH 7.4 |
| | Apparent | Apparent | Apparent |
| | K_{d} | K_{d} | K_{d} |
| Antibody ID | (PM) | (pM) | (pM) |
| Lib1_P1_A9_scFv | 51 | 18 | 205 |
| Lib1_P1_G3_scFv | 73 | 29 | 32 |
| 1675_Lib1_scFv | 77 | 30 | 52 |
| 7046_Lib8_scFv | 88 | 1160 | 2 |
| Lib1_P1_A6_scFv | 92 | 45 | 59 |
| 5802_Lib4_scFv | 101 | 1090 | 16 |
| 1374_Lib8_scFv | 116 | 43 | 61 |
| 9301_Lib8_scFv | 167 | 39 | 684 |
| 9621_Lib8_scFv | 217 | 2280 | 137 |
| 1405_Lib8_scFv | 305 | 389 | 39 |
| Lib4_P2_H3_scFv | 307 | 75 | 134 |
| 1081_Lib8_scFv | 391 | 274 | 167 |
| Lib4_P2_A5_scFv | 493 | 123 | 128 |
| Lib1_P1_D1_scFv | 589 | 338 | 187 |
| Lib1_P1_C6_scFv | 691 | 1430 | 697 |
| 4142_Lib1_scFv | 839 | 634 | 999 |
| Lib1_P1_E9_scFv | 915 | 639 | 536 |
| Lib8_P4_D4_scFv | 917 | 397 | 206 |
| 4166_Lib4_scFv | 958 | 567 | 380 |
| Lib4_P2_B4_scFv | 1070 | 225 | 96 |
| Lib8_P4_C11_scFv | 1080 | 617 | 155 |
| 3964_Lib4_scFv | 1130 | 737 | 689 |
| 3552_Lib8_scFv | 1160 | 412 | 18 |
| 2738_Lib1_scFv | 1180 | 519 | 922 |
| Lib4_P2_Al_scFv | 1200 | 235 | 31 |
| 189_Lib4_scFv | 1370 | 269 | 441 |
| Lib1_P1_Al_scFv | 1380 | 202 | 1080 |
| Lib6_P3_B9_scFv | 1410 | 1220 | 1030 |
| Lib6_P3_A1_scFv | 1410 | 1320 | 17 |
| Lib4_P2_A3_scFv | 1480 | 1930 | 387 |
| 487_Lib1_scFv | 1500 | 829 | 1510 |
| Lib6_P3_E10_scFv | 1720 | 659 | 1960 |
| 256_Lib8_scFv | 1760 | 731 | 758 |
| 4035_Lib1_scFv | 1760 | 1410 | 974 |
| 96_Lib4_scFv | 1770 | 1400 | 1210 |
| Lib1_P1_A2_scFv | 1780 | 76 | ND |
| 2301_Lib4_scFv | 1840 | 3870 | 803 |
| Lib4_P2_B1_scFv | 1890 | 2120 | 480 |
| Lib4_P2_A2_scFv | 2050 | 604 | 305 |
| 16_Lib8_scFv | 2110 | 1050 | ND |
| 4956_Lib1_scFv | 2220 | 1910 | 1360 |
| 11485_Lib1_scFv | 2220 | 1820 | 3160 |
| 399_Lib1_scFv | 2620 | 127 | 2050 |
| Lib4_P2_A10_scFv | 2700 | 3360 | 733 |
| Lib6_P3_A8_scFv | 2720 | 872 | 435 |
| 965_Lib4_scFv | 2790 | 621 | 1170 |
| 27_Lib4_scFv | 2850 | 443 | 656 |
| 15244_Lib8_scFv | 3050 | 1050 | 1910 |
| Lib4_P2_A11_scFv | 3110 | 33 | ND |
| Lib8_P4_C10_scFv | 3150 | 487 | 1010 |
| 8226_Lib8_scFv | 3170 | 1380 | 550 |
| 69_Lib8_scFv | 3700 | 859 | 1050 |
| 76_Lib4_scFv | 3910 | 366 | 1290 |
| 4675_Lib4_scFv | 4260 | 13600 | 2420 |
| 4029_Lib1_scFv | 4270 | 2580 | 4670 |
| 5662_Lib8_scFv | 4310 | 687 | 1360 |
| Lib1_P1_F10_scFv | 4970 | 15300 | 1500 |
| Lib6_P3_A4_scFv | 4970 | 520 | 3460 |
| Lib4_P2_D1_scFv | 5020 | 984 | 517 |
| Lib1_P1_A5_scFv | 6370 | 5020 | 1740 |
| 9312_Lib8_scFv | 6560 | 21600 | 1800 |
| 4761_Lib8_scFv | 7560 | 1330 | 2670 |
| 17561_Lib6_scFv | 9250 | 1790 | 5240 |

Binding to human VWF CK domain was measured at pH 7.4 and 5.5 and to cyno VWF CK domain at pH 7.4. Apparent K_{d} values estimated from the binding kinetics are listed. ND, not determined.

### Example 6: Recombinant Expression and Purification of Anti-VWF CK Domain for Binding Studies

For in vitro characterization studies, human and cyno VWF CK domains were modified with an N-terminal signal peptide derived from VWF followed by a hexa-histidine tag, StrepTagII, HRV-3C protease cleavage site, and three residues from VWF immediately N-terminal to the CK domain. In addition, a HA-tag was added to the C-terminus. Amino acid sequences are provided in Table 3.

TABLE 3
VWF CK domain constructs

| Construct Name | Construct Description | Amino Acid Sequence |
|---|---|---|
| His-StrepTagII- | Single-underlined residues | MIPARFAGVLLALALILPGTL |
| HRV3C-human | correspond to the N-terminal signal | CAEHHHHHHGSSAWSHPQF |
| VWF-CK-HA-tag | peptide sequence of VWF; bold | EKGSLEVLFQGPEPECNDITA |
| | residues correspond to a His-tag; | RLQYVKVGSCKSEVEVDIH |
| | double-underlined residues | YCQGKCASKAMYSIDINDV |
| | correspond to a StrepTagII tag; bold | QDQCSCCSPTRTEPMQVAL |
| | italic residues correspond to a HRV | HCTNGSVVYHEVLNAMEC |
| | 3C sequence; bold underlined | KCSPRKCSKGGGGSYPYDV |
| | residues correspond to the CK | PDYA |
| | domain of the human VWF protein; | (SEQ ID NO: 486) |
| | and bold and double-underlined | |
| | correspond to a C-terminal HA-tag | |
| His-StrepTagII- | Single-underlined residues | MIPARFAGVLLALALILPGTL |
| HRV3C-cyno | correspond to the N-terminal signal | CAEHHHHHHGSSAWSHPQF |
| VWF-CK-HA-tag | peptide sequence of VWF; bold | EKGSLEVLFQGPEPECSDITA |
| | residues correspond to a His-tag; | RLQYVKVGSCKSEVEVDIH |
| | double-underlined residues | YCQGKCASKAMYSIDINDV |
| | correspond to a | QDQCSCCSPTRTEPMQVPL |
| | StrepTagII tag; bold italic residues | HCTNGSVVYHEVLNAMQC |
| | correspond to a HRV 3C sequence; | ECSPRKCSKGGGGSYPYDVP |
| | bold underlined residues correspond | DYA |
| | to the CK domain of cyno VWF | (SEQ ID NO: 487) |
| | protein; and bold and double- | |
| | underlined correspond to a C- | |
| | terminal HA-tag | |

The corresponding DNA sequences were synthesized and inserted between the XbaI and AgeI restriction sites of the pcDNA3.4 vector. Expression was performed using Gibco CHO cells and a transient expression system essentially. Cells were maintained at 37° C. in a humidified atmosphere of 8% CO₂ with orbital shaking and sub-cultivated every 3-4 days in a cell medium prior to transfection.

Cells were transfected at a cell density of about 6 million cells/mL using a suitable reagent. For each 100 mL of cells, transfection was performed by diluting a total of 80 μL (1 mg/mL) DNA into 4 mL serum-free medium and combining this with 320 μL transfection reagent mixed with 3.7 mL serum-free medium. The mixture was then transferred to the cell culture. One day post-transfection, 600 μL transfection enhancer was added together with 24 mL culture feed. Cell culture supernatants were harvested 7-8 days post-transfection by centrifugation and followed by filtration.

Purification of expressed VWF CK domain was conducted by affinity chromatography utilizing the His-tag and standard purification methods. Briefly, clarified culture medium was loaded onto a HisTrap Excel column equilibrated in 20 mM HEPES, 100 mM NaCl, pH 7.4 (Buffer A). Following washing with 6% (v/v) Buffer B (20 mM HEPES, 100 mM NaCl, 500 mM Imidazole, pH 7.4) in Buffer A, bound protein was eluted with a linear gradient of 0-100% Buffer B. Purified protein was up-concentrated using a spin concentrator and subsequently dialyzed into 20 mM HEPES, 100 mM NaCl, pH 7.3.

### Example 7: Reformatting, Expression, and Purification of Anti-VWF CK Domain Antibodies

Using the VH and VL sequences from Table 1, antibodies were converted into desired formats for subsequent expression and purification. Table 4 lists the sequences of a selected set of antibodies configured to a monovalent (one-arm) human IgG4 format, also termed OA antibodies.

TABLE 4
Sequences of anti-VWF CK domain antibodies in monovalent (one-arm; OA) human IgG4 format

| | | Iso- | | | | SEQ |
|---|---|---|---|---|---|---|
| Antibody ID | Chain | type | Mutations | Segment | Sequence | ID NO |
| Lib1_P1_A9 | HC_trc | Human | S228P, | All | ESKYGPPCPPCPAPEAAGGPSVFLFPPKPKDT | 473 |
| OA | | IgG4 | F234A/L235A, | | LMISRTPEVTCVVVDVSQEDPEVQFNWYVD | |
| | | | T366W, delta- | | GVEVHNAKTKPREEQFNSTYRVVSVLTVLH | |
| | | | K447 | | QDWLNGKEYKCKVSNKGLPSSIEKTISKAKG | |
| | | | | | QPREPQVYTLPPSQEEMTKNQVSLWCLVKG | |
| | | | | | FYPSDIAVEWESNGQPENNYKTTPPVLDSDG | |
| | | | | | SFFLYSRLTVDKSRWQEGNVFSCSVMHEAL | |
| | | | | | HNHYTQKSLSLSLG | |
| | VH_HC | Human | S228P, | All | QVQLVQSGAEVKKPGASVKVSCKVSGDTLS | 453 |
| | | IgG4 | F234A/L235A, | | SYGIHWVRQAPGKGLEWMGGSIPSFGTAIYA | |
| | | | T366S/L368A/ | | QKFQGRVTMTEDTSTDTAYMELSSLKSEDT | |
| | | | Y407V, | | AVYYCTSEGPWLPFFSFDYWGQGTLVTVSS | |
| | | | delta-K447 | | ASTKGPSVFPLAPCSRSTSESTAALGCLVKD | |
| | | | | | YFPEPVTVSWNSGALTSGVHTFPAVLQSSGL | |
| | | | | | YSLSSVVTVPSSSLGTKTYTCNVDHKPSNTK | |
| | | | | | VDKRVESKYGPPCPPCPAPEAAGGPSVFLFPP | |
| | | | | | KPKDTLMISRTPEVTCVVVDVSQEDPEVQFN | |
| | | | | | WYVDGVEVHNAKTKPREEQFNSTYRVVSVL | |
| | | | | | TVLHQDWLNGKEYKCKVSNKGLPSSIEKTIS | |
| | | | | | KAKGQPREPQVYTLPPSQEEMTKNQVSLSC | |
| | | | | | AVKGFYPSDIAVEWESNGQPENNYKTTPPVL | |
| | | | | | DSDGSFFLVSRLTVDKSRWQEGNVFSCSVM | |
| | | | | | HEALHNHYTQKSLSLSLG | |
| | | | | CDR H1 | GDTLSSYG | 13 |
| | | | | CDR H2 | SIPSFGTA | 14 |
| | | | | CDR H3 | TSEGPWLPFFSFDY | 15 |
| | VL_LC | Human | — | All | DIQMTQSPSSVSASVGDRVTITCRASQGISRN | 463 |
| | | kappa | | | LAWYQQKPGKAPKLLIYKASSLASGVPSRFS | |
| | | | | | GSGSGTDFTLTISSLQPEDFANYYCQQSFSTP | |
| | | | | | VTFGGGTKVEIKRTVAAPSVFIFPPSDEQLKS | |
| | | | | | GTASVVCLLNNFYPREAKVQWKVDNALQS | |
| | | | | | GNSQESVTEQDSKDSTYSLSSTLTLSKADYE | |
| | | | | | KHKVYACEVTHQGLSSPVTKSFNRGEC | |
| | | | | CDR L1 | QGISRN | 17 |
| | | | | CDR L2 | KAS | |
| | | | | CDR L3 | QQSFSTPVT | 18 |
| Lib1_P1_G3 | HC_trc | Human | S228P, | All | ESKYGPPCPPCPAPEAAGGPSVFLFPPKPKDT | 474 |
| OA | | IgG4 | F234A/L235A, | | LMISRTPEVTCVVVDVSQEDPEVQFNWYVD | |
| | | | T366W, delta- | | GVEVHNAKTKPREEQFNSTYRVVSVLTVLH | |
| | | | K447 | | QDWLNGKEYKCKVSNKGLPSSIEKTISKAKG | |
| | | | | | QPREPQVYTLPPSQEEMTKNQVSLWCLVKG | |
| | | | | | FYPSDIAVEWESNGQPENNYKTTPPVLDSDG | |
| | | | | | SFFLYSRLTVDKSRWQEGNVFSCSVMHEAL | |
| | | | | | HNHYTQKSLSLSLG | |
| | VH_HC | Human | S228P | All | QVQLVQSGAEVKKPGASVKVSCKVSGYTSR | 454 |
| | | IgG4 | F234A/L235A, | | QYIHWVRQAPGKGLEWMGWVTPIFGTTNY | |
| | | | T366S/L368A/ | | AQKFQGRVTMTEDTSTDTAYMELSSLKSED | |
| | | | Y407V, | | TAVYYCAREWTGTTAFDIWGQGTLVTVSSA | |
| | | | delta-K447 | | STKGPSVFPLAPCSRSTSESTAALGCLVKDYF | |
| | | | | | PEPVTVSWNSGALTSGVHTFPAVLQSSGLYS | |
| | | | | | LSSVVTVPSSSLGTKTYTCNVDHKPSNTKVD | |
| | | | | | KRVESKYGPPCPPCPAPEAAGGPSVFLFPPKP | |
| | | | | | KDTLMISRTPEVTCVVVDVSQEDPEVQFNW | |
| | | | | | YVDGVEVHNAKTKPREEQFNSTYRVVSVLT | |
| | | | | | VLHQDWLNGKEYKCKVSNKGLPSSIEKTISK | |
| | | | | | AKGQPREPQVYTLPPSQEEMTKNQVSLSCA | |
| | | | | | VKGFYPSDIAVEWESNGQPENNYKTTPPVLD | |
| | | | | | SDGSFFLVSRLTVDKSRWQEGNVFSCSVMH | |
| | | | | | EALHNHYTQKSLSLSLG | |
| | | | | CDR H1 | GYTSRQY | 20 |
| | | | | CDR H2 | VTPIFGTT | 21 |
| | | | | CDR H3 | AREWTGTTAFDI | 22 |
| | VL_LC | Human | — | All | DIQMTQSPSSVSASVGDRVTITCRASQDIGHY | 464 |
| | | kappa | | | LAWYQQKPGKAPKLLIYAASNRASGVPSRFS | |
| | | | | | GSGSGTDFTLTISSLQPEDFANYYCQQGYSA | |
| | | | | | PYGFGGGTKVEIKRTVAAPSVFIFPPSDEQLK | |
| | | | | | SGTASVVCLLNNFYPREAKVQWKVDNALQS | |
| | | | | | GNSQESVTEQDSKDSTYSLSSTLTLSKADYE | |
| | | | | | KHKVYACEVTHQGLSSPVTKSFNRGEC | |
| | | | | CDR L1 | QDIGHY | 24 |
| | | | | CDR L2 | AAS | |
| | | | | CDR L3 | QQGYSAPYG | 25 |
| 7046_Lib8 | HC_trc | Human | S228P, | All | ESKYGPPCPPCPAPEAAGGPSVFLFPPKPKDT | 475 |
| OA | | IgG4 | F234A/L235A, | | LMISRTPEVTCVVVDVSQEDPEVQFNWYVD | |
| | | | T366W, delta- | | GVEVHNAKTKPREEQFNSTYRVVSVLTVLH | |
| | | | K447 | | QDWLNGKEYKCKVSNKGLPSSIEKTISKAKG | |
| | | | | | QPREPQVYTLPPSQEEMTKNQVSLWCLVKG | |
| | | | | | FYPSDIAVEWESNGQPENNYKTTPPVLDSDG | |
| | | | | | SFFLYSRLTVDKSRWQEGNVFSCSVMHEAL | |
| | | | | | HNHYTQKSLSLSLG | |
| | VH_HC | Human | S228P, | All | EVQLVESGGGLVQPGRSLRLSCAASGFNFHN | 455 |
| | | IgG4 | F234A/L235A, | | YDMHWVRQAPGKGLEWVSAISGSGYTADY | |
| | | | T366S/L368A/ | | ADSVEGRFTISRDNAKNSLYLQMNSLRAEDT | |
| | | | Y407V, | | AVYYCAKSHSSGWSSAFDIWGQGTMVTVSS | |
| | | | delta-K447 | | ASTKGPSVFPLAPCSRSTSESTAALGCLVKD | |
| | | | | | YFPEPVTVSWNSGALTSGVHTFPAVLQSSGL | |
| | | | | | YSLSSVVTVPSSSLGTKTYTCNVDHKPSNTK | |
| | | | | | VDKRVESKYGPPCPPCPAPEAAGGPSVFLFPP | |
| | | | | | KPKDTLMISRTPEVTCVVVDVSQEDPEVQFN | |
| | | | | | WYVDGVEVHNAKTKPREEQFNSTYRVVSVL | |
| | | | | | TVLHQDWLNGKEYKCKVSNKGLPSSIEKTIS | |
| | | | | | KAKGQPREPQVYTLPPSQEEMTKNQVSLSC | |
| | | | | | AVKGFYPSDIAVEWESNGQPENNYKTTPPVL | |
| | | | | | DSDGSFFLVSRLTVDKSRWQEGNVFSCSVM | |
| | | | | | HEALHNHYTQKSLSLSLG | |
| | | | | CDR H1 | GFNFHNYD | 34 |
| | | | | CDR H2 | ISGSGYTA | 35 |
| | | | | CDR H3 | AKSHSSGWSSAFDI | 36 |
| | VL_LC | Human | — | All | DIQMTQSPSSLSASVGDRVTITCRASQTIGYW | 465 |
| | | kappa | | | LAWYQQKPGKAPKLLIYDATNLATGVPSRF | |
| | | | | | SGSGSGTDFTLTISSLQPEDVATYYCQHYNP | |
| | | | | | YSQVTFGGGTKVEIKRTVAAPSVFIFPPSDEQ | |
| | | | | | LKSGTASVVCLLNNFYPREAKVQWKVDNAL | |
| | | | | | QSGNSQESVTEQDSKDSTYSLSSTLTLSKAD | |
| | | | | | YEKHKVYACEVTHQGLSSPVTKSFNRGEC | |
| | | | | CDR L1 | QTIGYW | 38 |
| | | | | CDR L2 | DAT | |
| | | | | CDR L3 | QHYNPYSQVT | 39 |
| Lib1_P1_A6 | HC_trc | Human | S228P, | All | ESKYGPPCPPCPAPEAAGGPSVFLFPPKPKDT | 476 |
| OA | | IgG4 | F234A/L235A, | | LMISRTPEVTCVVVDVSQEDPEVQFNWYVD | |
| | | | T366W, delta- | | GVEVHNAKTKPREEQFNSTYRVVSVLTVLH | |
| | | | K447 | | QDWLNGKEYKCKVSNKGLPSSIEKTISKAKG | |
| | | | | | QPREPQVYTLPPSQEEMTKNQVSLWCLVKG | |
| | | | | | FYPSDIAVEWESNGQPENNYKTTPPVLDSDG | |
| | | | | | SFFLYSRLTVDKSRWQEGNVFSCSVMHEAL | |
| | | | | | HNHYTQKSLSLSLG | |
| | VH_HC | Human | S228P, | All | QVQLVQSGAEVKKPGASVKVSCKVSGSTFS | 456 |
| | | IgG4 | F234A/L235A, | | NYAIHWVRQAPGKGLEWMGGIVPLSGTAIY | |
| | | | T366S/L368A/ | | AQKFQGRVTMTEDTSTDTAYMELSSLKSED | |
| | | | Y407V, | | TAVYYCARDQGESSGWYAIYYGMDVWGQ | |
| | | | delta-K447 | | GTTVTVSSASTKGPSVFPLAPCSRSTSESTAA | |
| | | | | | LGCLVKDYFPEPVTVSWNSGALTSGVHTFPA | |
| | | | | | VLQSSGLYSLSSVVTVPSSSLGTKTYTCNVD | |
| | | | | | HKPSNTKVDKRVESKYGPPCPPCPAPEAAGG | |
| | | | | | PSVFLFPPKPKDTLMISRTPEVTCVVVDVSQE | |
| | | | | | DPEVQFNWYVDGVEVHNAKTKPREEQFNST | |
| | | | | | YRVVSVLTVLHQDWLNGKEYKCKVSNKGL | |
| | | | | | PSSIEKTISKAKGQPREPQVYTLPPSQEEMTK | |
| | | | | | NQVSLSCAVKGFYPSDIAVEWESNGQPENN | |
| | | | | | YKTTPPVLDSDGSFFLVSRLTVDKSRWQEGN | |
| | | | | | VFSCSVMHEALHNHYTQKSLSLSLG | |
| | | | | CDR H1 | GSTFSNYA | 41 |
| | | | | CDR H2 | IVPLSGTA | 42 |
| | | | | CDR H3 | ARDQGESSGWYAIYYGMDV | 43 |
| | VL_LC | Human | — | All | DIQMTQSPSSVSASVGDRVTITCRASQGIGT | 466 |
| | | kappa | | | WLAWYQQKPGKAPKLLIYGSSSRASGVPSR | |
| | | | | | FSGSGSGTDFTLTISSLQPEDFANYYCQQSYG | |
| | | | | | KPTFGGGTKVEIKRTVAAPSVFIFPPSDEQLK | |
| | | | | | SGTASVVCLLNNFYPREAKVQWKVDNALQS | |
| | | | | | GNSQESVTEQDSKDSTYSLSSTLTLSKADYE | |
| | | | | | KHKVYACEVTHQGLSSPVTKSFNRGEC | |
| | | | | CDR L1 | QGIGTW | 45 |
| | | | | CDR L2 | GSS | |
| | | | | CDR L3 | QQSYGKPT | 46 |
| 1374_Lib8 | HC_trc | Human | S228P, | All | ESKYGPPCPPCPAPEAAGGPSVFLFPPKPKDT | 477 |
| OA | | IgG4 | F234A/L235A, | | LMISRTPEVTCVVVDVSQEDPEVQFNWYVD | |
| | | | T366W, delta- | | GVEVHNAKTKPREEQFNSTYRVVSVLTVLH | |
| | | | K447 | | QDWLNGKEYKCKVSNKGLPSSIEKTISKAKG | |
| | | | | | QPREPQVYTLPPSQEEMTKNQVSLWCLVKG | |
| | | | | | FYPSDIAVEWESNGQPENNYKTTPPVLDSDG | |
| | | | | | SFFLYSRLTVDKSRWQEGNVFSCSVMHEAL | |
| | | | | | HNHYTQKSLSLSLG | |
| | VH_HC | Human | S228P, | All | EVQLVESGGGLVQPGRSLRLSCAASGVDFST | 457 |
| | | IgG4 | F234A/L235A, | | YAMHWVRQAPGKGLEWVSAISSSGAYTDY | |
| | | | T366S/L368A/ | | ADSVEGRFTISRDNAKNSLYLQMNSLRAEDT | |
| | | | Y407V, | | AVYYCATDRGPFSGYGTQGWFDPWGQGTL | |
| | | | delta-K447 | | VTVSSASTKGPSVFPLAPCSRSTSESTAALGC | |
| | | | | | LVKDYFPEPVTVSWNSGALTSGVHTFPAVL | |
| | | | | | QSSGLYSLSSVVTVPSSSLGTKTYTCNVDHK | |
| | | | | | PSNTKVDKRVESKYGPPCPPCPAPEAAGGPS | |
| | | | | | VFLFPPKPKDTLMISRTPEVTCVVVDVSQED | |
| | | | | | PEVQFNWYVDGVEVHNAKTKPREEQFNSTY | |
| | | | | | RVVSVLTVLHQDWLNGKEYKCKVSNKGLPS | |
| | | | | | SIEKTISKAKGQPREPQVYTLPPSQEEMTKNQ | |
| | | | | | VSLSCAVKGFYPSDIAVEWESNGQPENNYKT | |
| | | | | | TPPVLDSDGSFFLVSRLTVDKSRWQEGNVFS | |
| | | | | | CSVMHEALHNHYTQKSLSLSLG | |
| | | | | CDR H1 | GVDFSTYA | 55 |
| | | | | CDR H2 | ISSSGAYT | 56 |
| | | | | CDR H3 | ATDRGPFSGYGTQGWFDP | 57 |
| | VL_LC | Human | — | All | DIQMTQSPSSLSASVGDRVTITCRASQTIRSY | 467 |
| | | kappa | | | LAWYQQKPGKAPKLLIYGASDLQSGVPSRFS | |
| | | | | | GSGSGTDFTLTISSLQPEDVATYYCQQYANF | |
| | | | | | PITFGGGTKVEIKRTVAAPSVFIFPPSDEQLKS | |
| | | | | | GTASVVCLLNNFYPREAKVQWKVDNALQS | |
| | | | | | GNSQESVTEQDSKDSTYSLSSTLTLSKADYE | |
| | | | | | KHKVYACEVTHQGLSSPVTKSFNRGEC | |
| | | | | CDR L1 | QTIRSY | 59 |
| | | | | CDR L2 | GAS | |
| | | | | CDR L3 | QQYANFPIT | 60 |
| 1081_Lib8 | HC_trc | Human | S228P, | All | ESKYGPPCPPCPAPEAAGGPSVFLFPPKPKDT | 478 |
| OA | | IgG4 | F234A/L235A, | | LMISRTPEVTCVVVDVSQEDPEVQFNWYVD | |
| | | | T366W, delta- | | GVEVHNAKTKPREEQFNSTYRVVSVLTVLH | |
| | | | K447 | | QDWLNGKEYKCKVSNKGLPSSIEKTISKAKG | |
| | | | | | QPREPQVYTLPPSQEEMTKNQVSLWCLVKG | |
| | | | | | FYPSDIAVEWESNGQPENNYKTTPPVLDSDG | |
| | | | | | SFFLYSRLTVDKSRWQEGNVFSCSVMHEAL | |
| | | | | | HNHYTQKSLSLSLG | |
| | VH_HC | Human | S228P, | All | EVQLVESGGGLVQPGRSLRLSCAASGENFHN | 458 |
| | | IgG4 | F234A/L235A, | | YDMHWVRQAPGKGLEWVSAISGGAVSTDY | |
| | | | T366S/L368A/ | | ADSVEGRFTISRDNAKNSLYLQMNSLRAEDT | |
| | | | Y407V, | | AVYYCARSSWYGGQIDFWGQGTLVTVSSAS | |
| | | | delta-K447 | | TKGPSVFPLAPCSRSTSESTAALGCLVKDYFP | |
| | | | | | EPVTVSWNSGALTSGVHTFPAVLQSSGLYSL | |
| | | | | | SSVVTVPSSSLGTKTYTCNVDHKPSNTKVDK | |
| | | | | | RVESKYGPPCPPCPAPEAAGGPSVFLFPPKPK | |
| | | | | | DTLMISRTPEVTCVVVDVSQEDPEVQFNWY | |
| | | | | | VDGVEVHNAKTKPREEQFNSTYRVVSVLTV | |
| | | | | | LHQDWLNGKEYKCKVSNKGLPSSIEKTISKA | |
| | | | | | KGQPREPQVYTLPPSQEEMTKNQVSLSCAV | |
| | | | | | KGFYPSDIAVEWESNGQPENNYKTTPPVLDS | |
| | | | | | DGSFFLVSRLTVDKSRWQEGNVFSCSVMHE | |
| | | | | | ALHNHYTQKSLSLSLG | |
| | | | | CDR H1 | GFNFHNYD | 90 |
| | | | | CDR H2 | ISGGAVST | 91 |
| | | | | CDR H3 | ARSSWYGGQIDF | 92 |
| | VL_LC | Human | — | All | DIQMTQSPSSLSASVGDRVTITCRASESVSSW | 468 |
| | | kappa | | | LAWYQQKPGKAPKLLIYGASTLEGGVPSRFS | |
| | | | | | GSGSGTDFTLTISSLQPEDVATYYCQQAYRT | |
| | | | | | PVTFGGGTKVEIKRTVAAPSVFIFPPSDEQLK | |
| | | | | | SGTASVVCLLNNFYPREAKVQWKVDNALQS | |
| | | | | | GNSQESVTEQDSKDSTYSLSSTLTLSKADYE | |
| | | | | | KHKVYACEVTHQGLSSPVTKSFNRGEC | |
| | | | | CDR L1 | ESVSSW | 94 |
| | | | | CDR L2 | GAS | |
| | | | | CDR L3 | QQAYRTPVT | 95 |
| Lib1_P1_C6 | HC_trc | Human | S228P, | All | ESKYGPPCPPCPAPEAAGGPSVFLFPPKPKDT | 479 |
| OA | | IgG4 | F234A/L235A, | | LMISRTPEVTCVVVDVSQEDPEVQFNWYVD | |
| | | | T366W, delta- | | GVEVHNAKTKPREEQFNSTYRVVSVLTVLH | |
| | | | K447 | | QDWLNGKEYKCKVSNKGLPSSIEKTISKAKG | |
| | | | | | QPREPQVYTLPPSQEEMTKNQVSLWCLVKG | |
| | | | | | FYPSDIAVEWESNGQPENNYKTTPPVLDSDG | |
| | | | | | SFFLYSRLTVDKSRWQEGNVFSCSVMHEAL | |
| | | | | | HNHYTQKSLSLSLG | |
| | VH_HC | Human | S228P, | All | QVQLVQSGAEVKKPGASVKVSCKVSGYTFA | 459 |
| | | IgG4 | F234A/L235A, | | SHYIHWVRQAPGKGLEWMGGMDPATGQTI | |
| | | | T366S/L368A/ | | YAQKFQGRVTMTEDTSTDTAYMELSSLKSE | |
| | | | Y407V, | | DTAVYYCAKGDRSSWYEVLSFQHWGQGTL | |
| | | | delta-K447 | | VTVSSASTKGPSVFPLAPCSRSTSESTAALGC | |
| | | | | | LVKDYFPEPVTVSWNSGALTSGVHTFPAVL | |
| | | | | | QSSGLYSLSSVVTVPSSSLGTKTYTCNVDHK | |
| | | | | | PSNTKVDKRVESKYGPPCPPCPAPEAAGGPS | |
| | | | | | VFLFPPKPKDTLMISRTPEVTCVVVDVSQED | |
| | | | | | PEVQFNWYVDGVEVHNAKTKPREEQFNSTY | |
| | | | | | RVVSVLTVLHQDWLNGKEYKCKVSNKGLPS | |
| | | | | | SIEKTISKAKGQPREPQVYTLPPSQEEMTKNQ | |
| | | | | | VSLSCAVKGFYPSDIAVEWESNGQPENNYKT | |
| | | | | | TPPVLDSDGSFFLVSRLTVDKSRWQEGNVFS | |
| | | | | | CSVMHEALHNHYTQKSLSLSLG | |
| | | | | CDR H1 | GYTFASHY | 111 |
| | | | | CDR H2 | MDPATGQT | 112 |
| | | | | CDR H3 | AKGDRSSWYEVLSFQH | 113 |
| | VL_LC | Human | — | All | DIQMTQSPSSVSASVGDRVTITCRASQGISTD | 469 |
| | | kappa | | | LAWYQQKPGKAPKLLIYDASILQRGVPSRFS | |
| | | | | | GSGSGTDFTLTISSLQPEDFANYYCQQVRTY | |
| | | | | | PFTFGGGTKVEIKRTVAAPSVFIFPPSDEQLK | |
| | | | | | SGTASVVCLLNNFYPREAKVQWKVDNALQS | |
| | | | | | GNSQESVTEQDSKDSTYSLSSTLTLSKADYE | |
| | | | | | KHKVYACEVTHQGLSSPVTKSFNRGEC | |
| | | | | CDR L1 | QGISTD | 115 |
| | | | | CDR L2 | DAS | |
| | | | | CDR L3 | QQVRTYPFT | 116 |
| 4035_Lib1 | HC_trc | Human | S228P, | All | ESKYGPPCPPCPAPEAAGGPSVFLFPPKPKDT | 480 |
| OA | | IgG4 | F234A/L235A, | | LMISRTPEVTCVVVDVSQEDPEVQFNWYVD | |
| | | | T366W, delta- | | GVEVHNAKTKPREEQFNSTYRVVSVLTVLH | |
| | | | K447 | | QDWLNGKEYKCKVSNKGLPSSIEKTISKAKG | |
| | | | | | QPREPQVYTLPPSQEEMTKNQVSLWCLVKG | |
| | | | | | FYPSDIAVEWESNGQPENNYKTTPPVLDSDG | |
| | | | | | SFFLYSRLTVDKSRWQEGNVFSCSVMHEAL | |
| | | | | | HNHYTQKSLSLSLG | |
| | VH_HC | Human | S228P, | All | QVQLVQSGAEVKKPGASVKVSCKVSGYAFP | 460 |
| | | IgG4 | F234A/L235A, | | SYGIHWVRQAPGKGLEWMGGISAFTGDTIY | |
| | | | T366S/L368A/ | | AQKFQGRVTMTEDTSTDTAYMELSSLKSED | |
| | | | Y407V, | | TAVYYCASGPTNVLTGLMESAEYFQHWGQ | |
| | | | delta-K447 | | GTLVTVSSASTKGPSVFPLAPCSRSTSESTAA | |
| | | | | | LGCLVKDYFPEPVTVSWNSGALTSGVHTFPA | |
| | | | | | VLQSSGLYSLSSVVTVPSSSLGTKTYTCNVD | |
| | | | | | HKPSNTKVDKRVESKYGPPCPPCPAPEAAGG | |
| | | | | | PSVFLFPPKPKDTLMISRTPEVTCVVVDVSQE | |
| | | | | | DPEVQFNWYVDGVEVHNAKTKPREEQFNST | |
| | | | | | YRVVSVLTVLHQDWLNGKEYKCKVSNKGL | |
| | | | | | PSSIEKTISKAKGQPREPQVYTLPPSQEEMTK | |
| | | | | | NQVSLSCAVKGFYPSDIAVEWESNGQPENN | |
| | | | | | YKTTPPVLDSDGSFFLVSRLTVDKSRWQEGN | |
| | | | | | VFSCSVMHEALHNHYTQKSLSLSLG | |
| | | | | CDR H1 | GYAFPSYG | 244 |
| | | | | CDR H2 | ISAFTGDT | 245 |
| | | | | CDR H3 | ASGPTNVLTGLMESAEYFQH | 246 |
| | VL_LC | Human | — | All | DIQMTQSPSSVSASVGDRVTITCRASQGISRD | 470 |
| | | kappa | | | LAWYQQKPGKAPKLLIYGASGRATGVPSRF | |
| | | | | | SGSGSGTDFTLTISSLQPEDFANYYCQQSYIT | |
| | | | | | PTTFGGGTKVEIKRTVAAPSVFIFPPSDEQLK | |
| | | | | | SGTASVVCLLNNFYPREAKVQWKVDNALQS | |
| | | | | | GNSQESVTEQDSKDSTYSLSSTLTLSKADYE | |
| | | | | | KHKVYACEVTHQGLSSPVTKSFNRGEC | |
| | | | | CDR L1 | QGISRD | 248 |
| | | | | CDR L2 | GAS | |
| | | | | CDR L3 | QQSYITPTT | 249 |
| 399_Lib1 OA | HC_trc | Human | S228P, | All | ESKYGPPCPPCPAPEAAGGPSVFLFPPKPKDT | 481 |
| | | IgG4 | F234A/L235A, | | LMISRTPEVTCVVVDVSQEDPEVQFNWYVD | |
| | | | T366W, delta- | | GVEVHNAKTKPREEQFNSTYRVVSVLTVLH | |
| | | | K447 | | QDWLNGKEYKCKVSNKGLPSSIEKTISKAKG | |
| | | | | | QPREPQVYTLPPSQEEMTKNQVSLWCLVKG | |
| | | | | | FYPSDIAVEWESNGQPENNYKTTPPVLDSDG | |
| | | | | | SFFLYSRLTVDKSRWQEGNVFSCSVMHEAL | |
| | | | | | HNHYTQKSLSLSLG | |
| | VH_HC | Human | S228P, | All | QVQLVQSGAEVKKPGASVKVSCKVSGITFTS | 461 |
| | | IgG4 | F234A/L235A, | | DYIHWVRQAPGKGLEWMGGMNPYSNKAIY | |
| | | | T366S/L368A/ | | AQKFQGRVTMTEDTSTDTAYMELSSLKSED | |
| | | | Y407V, | | TAVYYCATGGLRIAARTSYFDYWGQGTLVT | |
| | | | delta-K447 | | VSSASTKGPSVFPLAPCSRSTSESTAALGCLV | |
| | | | | | KDYFPEPVTVSWNSGALTSGVHTFPAVLQSS | |
| | | | | | GLYSLSSVVTVPSSSLGTKTYTCNVDHKPSN | |
| | | | | | TKVDKRVESKYGPPCPPCPAPEAAGGPSVFL | |
| | | | | | FPPKPKDTLMISRTPEVTCVVVDVSQEDPEV | |
| | | | | | QFNWYVDGVEVHNAKTKPREEQFNSTYRV | |
| | | | | | VSVLTVLHQDWLNGKEYKCKVSNKGLPSSI | |
| | | | | | EKTISKAKGQPREPQVYTLPPSQEEMTKNQV | |
| | | | | | SLSCAVKGFYPSDIA VEWESNGQPENNYKTT | |
| | | | | | PPVLDSDGSFFLVSRLTVDKSRWQEGNVFSC | |
| | | | | | SVMHEALHNHYTQKSLSLSLG | |
| | | | | CDR H1 | GITFTSDY | 307 |
| | | | | CDR H2 | MNPYSNKA | 308 |
| | | | | CDR H3 | ATGGLRIAARTSYFDY | 309 |
| | VL LC | Human | — | All | DIQMTQSPSSVSASVGDRVTITCRASMDIAGS | 471 |
| | | kappa | | | LAWYQQKPGKAPKLLIYDTSFRATGVPSRFS | |
| | | | | | GSGSGTDFTLTISSLQPEDFANYYCQQYDNP | |
| | | | | | PRLTFGGGTKVEIKRTVAAPSVFIFPPSDEQL | |
| | | | | | KSGTASVVCLLNNFYPREAKVQWKVDNAL | |
| | | | | | QSGNSQESVTEQDSKDSTYSLSSTLTLSKAD | |
| | | | | | YEKHKVYACEVTHQGLSSPVTKSFNRGEC | |
| | | | | CDR L1 | MDIAGS | 311 |
| | | | | CDR L2 | DTS | |
| | | | | CDR L3 | QQYDNPPRLT | 312 |
| 4029_Lib1 | HC_trc | Human | S228P, | All | ESKYGPPCPPCPAPEAAGGPSVFLFPPKPKDT | 482 |
| OA | | IgG4 | F234A/L235A, | | LMISRTPEVTCVVVDVSQEDPEVQFNWYVD | |
| | | | T366W, delta- | | GVEVHNAKTKPREEQFNSTYRVVSVLTVLH | |
| | | | K447 | | QDWLNGKEYKCKVSNKGLPSSIEKTISKAKG | |
| | | | | | QPREPQVYTLPPSQEEMTKNQVSLWCLVKG | |
| | | | | | FYPSDIAVEWESNGQPENNYKTTPPVLDSDG | |
| | | | | | SFFLYSRLTVDKSRWQEGNVFSCSVMHEAL | |
| | | | | | HNHYTQKSLSLSLG | |
| | VH_HC | Human | S228P, | All | QVQLVQSGAEVKKPGASVKVSCKVSGYPFT | 462 |
| | | IgG4 | F234A/L235A, | | SSGIHWVRQAPGKGLEWMGGVIPLFGTANY | |
| | | | T366S/L368A/ | | AQKFQGRVTMTEDTSTDTAYMELSSLKSED | |
| | | | Y407V, | | TAVYYCARIRVSSGWYRDYGMDVWGQGTT | |
| | | | delta-K447 | | VTVSSASTKGPSVFPLAPCSRSTSESTAALGC | |
| | | | | | LVKDYFPEPVTVSWNSGALTSGVHTFPAVL | |
| | | | | | QSSGLYSLSSVVTVPSSSLGTKTYTCNVDHK | |
| | | | | | PSNTKVDKRVESKYGPPCPPCPAPEAAGGPS | |
| | | | | | VFLFPPKPKDTLMISRTPEVTCVVVDVSQED | |
| | | | | | PEVQFNWYVDGVEVHNAKTKPREEQFNSTY | |
| | | | | | RVVSVLTVLHQDWLNGKEYKCKVSNKGLPS | |
| | | | | | SIEKTISKAKGQPREPQVYTLPPSQEEMTKNQ | |
| | | | | | VSLSCAVKGFYPSDIAVEWESNGQPENNYKT | |
| | | | | | TPPVLDSDGSFFLVSRLTVDKSRWQEGNVFS | |
| | | | | | CSVMHEALHNHYTQKSLSLSLG | |
| | | | | CDR H1 | GYPFTSSG | 391 |
| | | | | CDR H2 | VIPLFGTA | 392 |
| | | | | CDR H3 | ARIRVSSGWYRDYGMDV | 393 |
| | VL_LC | Human | — | All | DIQMTQSPSSVSASVGDRVTITCRASQSIGDF | 472 |
| | | kappa | | | LAWYQQKPGKAPKLLIYDASHLQTGVPSRFS | |
| | | | | | GSGSGTDFTLTISSLQPEDFANYYCQQTSTIPP | |
| | | | | | TFGGGTKVEIKRTVAAPSVFIFPPSDEQLKSG | |
| | | | | | TASVVCLLNNFYPREAKVQWKVDNALQSG | |
| | | | | | NSQESVTEQDSKDSTYSLSSTLTLSKADYEK | |
| | | | | | HKVYACEVTHQGLSSPVTKSFNRGEC | |
| | | | | CDR L1 | QSIGDF | 395 |
| | | | | CDR L2 | DAS | |
| | | | | CDR L3 | QQTSTIPPT | 396 |

Abbreviations:
HC_trc, truncated heavy chain; VH_HC, heavy chain containing VH; VL_LC, light chain containing VL.

Here, the full-length HC contains S228P, F234A/L235A, T366S/L368A/Y407V, and delta-K447 mutations. The truncated HC (HC_trc) starts at residue 216 (EU numbering) and contains S228P, F234A/L235A, T366W, and delta-K447 mutations. The LC is human kappa. The T366W and T366S/L368A/Y407V mutations are the so-called knob/hole mutations that facilitate hetero-dimer assembly, i.e., pairing of HC and HC_trc, and the F234A/L235A mutations are effector silencing mutations. As will be appreciated by a person of skill in the art, the knob/hole mutations can be interchanged between the HC and HC_trc, and mutations can be omitted or combined as needed.

Each of the three antibody chains (HC, HC_trc, and LC) were synthesized as DNA and cloned into the pcDNA3.1 vector. The expression of antibodies was performed using the CHO cells. Cells were expanded from a working cell bank and passaged in serum-free, protein-free, animal origin-free medium. Cells were cultured for 14 days, and the harvest was performed by filtration using 0.22 μm filtration units and DE as filter aid, after which antibodies were purified using standard affinity chromatographic methods.

### Example 8: Binding of Anti-VWF CK Domain Antibodies to Recombinant Human and Cynomolgus Monkey VWF CK Domain

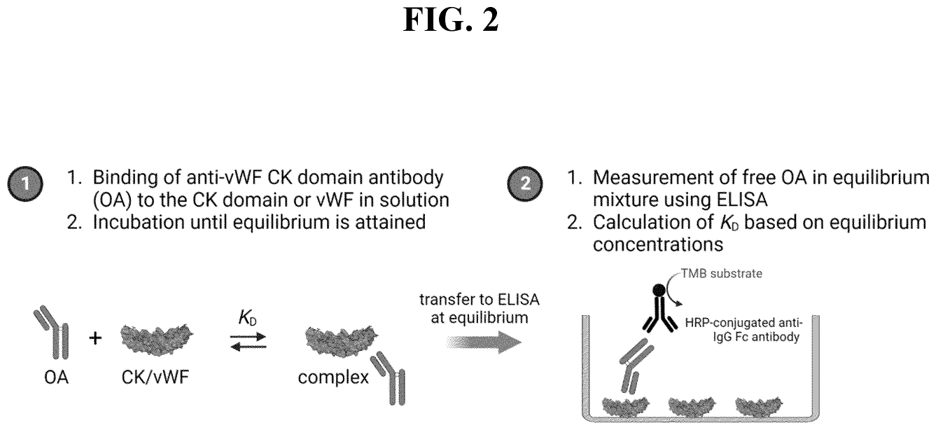

To determine the affinity of monovalent anti-VWF CK domain antibodies for the recombinant human and cyno CK domains and human plasma-derived VWF (e.g., free of FVIII), a solution-phase binding assay was performed. In this assay, a concentration range of antibody (0-140 nM) was allowed to bind to a fixed concentration (10 or 20 nM) of the CK domain or plasma-derived VWF in pH 7.4 (20 mM HEPES, 150 mM NaCl, 5 mM CaCl₂, 1% (w/w) BSA, pH 7.4) or pH 5.5 (20 mM MES, 150 mM NaCl, 5 mM CaCl₂, 1% (w/w) BSA, pH 5.5) buffer. Following incubation for 30 min at room temperature to establish equilibrium, unbound antibody was quantified by ELISA using the immobilized human CK domain for capture and HRP-conjugated goat anti-human IgG Fc antibody for detection. By relating the measured ELISA signal to a standard curve prepared with defined concentrations of the same antibody in absence of CK or VWF, the concentration of unbound antibody in the equilibrium mixtures was determined. Incubation times in the ELISA were optimized to ensure minimal perturbation of the equilibrium between antibody and CK or VWF. See FIG. 2 for an outline of the assay.

The ELISA was prepared by coating a 96-well plate with 100 μL 2 μg/mL human VWF CK domain in TBS (10 mM Tris-HCl, 150 mM NaCl, pH 7.5) overnight at 5° C. Following a single wash of the plate with 200 μL of wash buffer (TBS containing 0.05% (v/v) Tween-20 and 5 mM CaCl₂), the plate was blocked by incubation with 200 μL blocking buffer (TBS containing 5 mM CaCl₂ and 1% (w/w) BSA) for 1 hour at room temperature. Before transfer of 100 μL of free antibody or antibody/CK/VWF equilibrium mixture to the plate, it was washed with 200 μL wash buffer. Incubation lasted 3 min and was followed by 5-min incubation with 100 μL HRP-conjugated goat anti-human IgG Fc antibody for detection (1 mg/mL stock diluted 1:20,000 in blocking buffer). Before and after addition of a detection antibody, the plate was washed 5 times with 200 μL wash buffer. A chromogenic horseradish peroxidase (HRP) substrate (100 μL) was then added and following color development for 3 min, 100 μL ELISA stop solution were added to quench the reaction. Absorbance was measured at 450 nm with subtraction of the absorbance at 620 nm.

The standard curve prepared with free antibody was fitted to a Sigmoidal four-parameter logistic (4PL) model. Using the standard curve, the ELISA readouts for the equilibrium mixtures were converted to concentrations of unbound antibody. For this conversion, only equilibrium mixtures yielding ELISA readouts mapping onto the mid-to-lower part of the standard curve were selected. From the concentration of unbound antibody an apparent equilibrium dissociation constant (K_{d}) could be calculated using Eq. 1:

$${K}_{D} = \frac{{OA}_{free} \times ( {VWFCK}_{total} - ( {OA}_{total} - {OA}_{free} ) )}{{VWFCK}_{total} - {OA}_{free}} \qquad ( Eq . 1 )$$

Here, OAₜₒₜₐₗ is the total molar concentration of antibody, OA_{free} is the concentration of unbound antibody determined by ELISA, VWFCKₜₒₜₐₗ is the concentration of antibody binding sites on the CK domain or VWF. For the CK domain, the number of binding sites is twice the molar concentration of the CK dimer, while the number of binding sites on VWF equals its monomer concentration.

Apparent K_{d}-values for a subset of monovalent antibodies are listed in Table 5.

TABLE 5
Apparent K_{d}-values for the binding of one-arm (OA) anti-VWF CK antibodies to
recombinant human or cyno CK domain or plasma-derived VWF (pd-VWF)

| | Apparent K_{d} (nM) | | | | |
|---|---|---|---|---|---|
| | Human | Human | Cyno | Human | Cyno |
| | VWF at | CK at | CK at | CK at | CK at |
| Antibody ID | pH 7.4 | pH 7.4 | pH 7.4 | pH 5.5 | pH 5.5 |
| Lib1_P1_A9 OA | 5.7 ± 0.8 | 5.3 ± 0.7 | 10.2 ± 0.6 | 17.9 ± 5.6 | 26.4 ± 0.3 |
| Lib1_P1_G3 OA | 5.3 ± 1.4 | 4.3 ± 0.9 | 11.0 ± 0.9 | 32.3 ± 1.0 | no binding |
| Lib1_P1_A6 OA | 2.7 ± 0.6 | 2.7 ± 0.4 | 3.4 ± 0.6 | 8.2 ± 0.8 | 13.0 ± 0.3 |
| 399_Lib1 OA | ND | 13.0 ± 0.7 | 16.8 ± 0.9 | ND | ND |

Results are shown as mean ± standard deviation (n = 2).
Abbreviations: ND, not determined; Cyno, Cynomolgus monkey

For all antibodies, binding to the recombinant human CK domain and human plasma-derived VWF at pH 7.4 was similar, indicating that epitopes are fully accessible when the CK domain is embedded in VWF. Binding to the cyno CK domain was retained for all antibodies with less than 2-fold change in K_{d} value as compared to the human counterpart.

### Example 9: Mapping of Hot Spot Residues for Antibody Binding on the Human CK Domain

Amino acid residues in the VWF CK domain important for antibody binding (so-called hot spot residues) were identified by alanine-scanning mutagenesis. A panel of alanine substitution variants of the human recombinant CK domain were produced. Positions selected for alanine substitution were all surface exposed and selected based on inspection of the crystal structure of the CK domain.

Binding was assessed using a direct ELISA system. The ELISA was prepared by coating of a 96-well plate with 100 μL (2 μg/mL) of each of human VWF CK domain alanine variants in TBS (10 mM Tris-HCl, 150 mM NaCl, pH 7.5) overnight at 5° C. Following a single wash of the plate with 200 μL of wash buffer (TBS containing 0.05% (v/v) Tween-20 and 5 mM CaCl₂), the plate was blocked by incubation with 200 μL blocking buffer (TBS containing 5 mM CaCl₂ and 1% BSA) for 1 hour at room temperature. Before transfer of 100 μL of monovalent (one-arm) antibody at 20 nM, 5 nM, or 0.5 nM in 20 mM HEPES, 150 mM NaCl, 5 mM CaCl₂, 1% (w/w) BSA, pH 7.4 buffer, the plate was washed with 200 μL wash buffer. Incubation with the antibody lasted 45 min and was followed by a 5-min incubation with 100 μL HRP-conjugated goat anti-human IgG Fc antibody for detection (1 mg/mL stock diluted 1:20,000 in blocking buffer). Before and after addition of detection antibody, the plate was washed 5 times with 200 μL wash buffer. An HRP-based chromogenic substrate (100 μL) was then added and, following color development for 3 min, 100 μL ELISA stop solution were added to quench the reaction. Absorbance was measured at 450 nm with subtraction of the absorbance at 620 nm. Amino acid positions important for binding of a given antibody were defined as those in which the measured absorbance was ≤33% of the absorbance measured for the binding of the same antibody to the wild-type human VWF CK domain. Results are shown in Table 6.

TABLE 6
Monovalent (one-arm) antibody binding to alanine substitution variant of the human VWF CK domain

| Antibody ID | D2726A | R2730A | K2735A | E2742A | E2744A | D2746A | K2757A | D2763A | D2766A |
|---|---|---|---|---|---|---|---|---|---|
| Lib1_P1_A9 OA | X | X | | | | X | X | | |
| Lib1_P1_G3 OA | | X | X | | X | X | | | |
| 7046_Lib8 OA | | | X | | | X | X | | |
| Lib1_P1_A6 OA | | X | | | | X | X | | |
| 1374_Lib8 OA | | | X | | X | X | | X | |
| 1081_Lib8 OA | | X | X | | X | X | X | X | |
| Lib1_P1_C6 OA | | | X | | | | | | |
| 4035_Lib1 OA | | | X | | | | | | |
| 399_Lib1 OA | X | | X | | | | X | | |
| 4029_Lib1 OA | | | X | | | | | X | |

| Antibody ID | R2778A | E2780A | T2789A | Y2795A | E2797A | K2805A | K2810A |
|---|---|---|---|---|---|---|---|
| Lib1_P1_A9 OA | | | | | | | |
| Lib1_P1_G3 OA | | | X | | | | |
| 7046_Lib8 OA | | | | | | | |
| Lib1_P1_A6 OA | | | | | | | |
| 1374_Lib8 OA | | | | | | | |
| 1081_Lib8 OA | | | | | | | |
| Lib1_P1_C6 OA | | | | | | | |
| 4035_Lib1 OA | | | | | | | |
| 399_Lib1 OA | | | | | | | |
| 4029_Lib1 OA | | | | | | | |

Substitutions resulting in ≤33% binding compared to the wild-type CK domain at 0.5, 5 or 20 nM monovalent (OA) antibody are marked by an ‘X’ and define a hot spot.

### Example 10: Effect of Anti-VWF CK Domain Antibodies on the VWF:FVIII Interaction

VWF is a carrier of coagulation Factor VIII (FVIII) in circulation and is critical for maintaining its half-life and concentration in plasma. To investigate the effect of antibody binding on the VWF:FVIII interaction, the capacity of VWF to bind FVIII in the presence of monovalent (one-arm) anti-VWF CK domain antibody was determined. To this end, the VWF antigen level in plasma from 5 healthy human donors was determined using a VWF:Ag immuno-turbidimetric assay. Plasma samples (stabilized in 3.2% sodium citrate) were then incubated for 17 hours at 37° C. in the presence of 0 nM (“untreated”), 200 nM, or 600 nM antibody concentrations followed by dilution of plasma to 0.1 IU/mL (10%) VWF and measurement of the FVIII binding capacity of VWF using a quantitative VWF:FVIIIB enzyme immunoassay. In this assay, VWF from plasma was captured using a rabbit anti-human VWF F(ab′)2 fragment followed by detection of bound FVIII using a peroxidase-conjugated mouse anti-human FVIII monoclonal antibody for detection.

Results are shown in Table 7. For all antibodies, the mean level of VWF:FVIII was greater than 80% of the level in untreated donor-matched plasma. The results demonstrate that the tested antibodies minimally affected the capacity of VWF to bind FVIII.

TABLE 7
VWF binding to FVIII following incubation of plasma
with one-arm anti-VWF CK domain antibodies

| | Antibody | |
|---|---|---|
| | concentration | VWF:FVIII level |
| Antibody ID | (nM) | (% of control) |
| Lib1_P1_A9 OA | 200 | 91.1 ± 3.8 |
| Lib1_P1_A9 OA | 600 | 93.9 ± 7.1 |
| Lib1_P1_A6 OA | 200 | 89.7 ± 4.8 |
| Lib1_P1_A6 OA | 600 | 88.6 ± 8.3 |
| 1081_Lib8 OA | 200 | 93.2 ± 7.4 |
| 1081_Lib8 OA | 600 | 85.1 ± 5.3 |
| 7046_Lib8 OA | 200 | 95.0 ± 4.6 |
| 7046_Lib8 OA | 600 | 90.7 ± 4.2 |
| Lib1_P1_G3 OA | 200 | 84.5 ± 4.7 |
| Lib1_P1_G3 OA | 600 | 85.8 ± 8.6 |

Measured binding levels were normalized to untreated plasma from the same donor. Results are shown as mean±standard deviation for plasma from 5 healthy human donors.

### Example 11: Effect of Anti-VWF CK Domain Antibodies on the VWF:Platelet gp1b Interaction

Glycoprotein Ib (gp1b) is a key receptor for VWF on the platelet. The interaction with gp1b is dependent upon the exposure of a binding site in the A1 domain of VWF. Under physiological conditions, the gp1b binding site becomes accessible when VWF is subjected to shear stress at sites of vascular injury. Under static conditions, such as those prevailing in vitro, the effect of shear stress can be recapitulated by addition of the peptide antibiotic ristocetin, which enhances the binding of VWF to gp1b receptors on platelets.

To evaluate the effect of monovalent (one-arm) anti-VWF CK domain antibodies on the ability of VWF to engage the gp1b receptor on platelets, plasma from three healthy human donors (stabilized in 3.2% sodium citrate) was incubated with 0 nM (“untreated”), 200 nM, 600 nM, or 1000 nM of antibody for 10-30 mins followed by quantitative determination of the ristocetin cofactor activity of VWF using the STA-VWF:RCo assay. This assay is based on the change in turbidity of a platelet suspension, as measured by photometry. A suspension of reconstituted lyophilized platelets is added to the test plasma followed by the addition of ristocetin to induce VWF- and gp1b-dependent platelet aggregation. Aggregation induces a decrease in turbidity and, consequently, a decrease in absorbance, which can be measured photometrically. The decrease in absorbance is a function of the VWF activity in the plasma sample.

For each combination of donor plasma, antibody, and antibody concentration, the ristocetin cofactor activity of VWF was measured and normalized to that of a donor-matched control plasma without antibody. As shown in Table 8, VWF retains the ability to bind platelet gp1b in the presence of Lib1_P1_A9 and Lib1_P1_A6 monovalent antibodies.

TABLE 8
Ristocetin cofactor activity of VWF (VWF:Rco)
following incubation of plasma with one-arm
anti-VWF CK antibodies

| | Antibody | |
|---|---|---|
| | concentration | VWF:RCo |
| Antibody ID | (nM) | (% of control) |
| Lib1_P1_A9 OA | 1000 | 102 ± 10 |
| Lib1_P1_A9 OA | 600 | 107 ± 8 |
| Lib1_P1_A9 OA | 200 | 109 ± 9 |
| Lib1_P1_A6 OA | 1000 | 109 ± 5 |
| Lib1_P1_A6 OA | 600 | 103 ± 13 |
| Lib1_P1_A6 OA | 200 | 104 ± 2 |

Measured activity levels were normalized to the activity of untreated plasma from the same donor. Results are shown as mean±standard deviation for plasma from 3 healthy human donors.

### Example 12: Effect of Anti-VWF CK Domain Antibodies on the Cleavage of VWF by ADAMTS13

The plasma protease ADAM Metallopeptidase With Thrombospondin Type 1 Motif 13 (ADAMTS13) is responsible for processing of VWF into shorter multimers. The cleavage site for ADAMTS13 within the VWF A2 domain is cryptic under static conditions and is exposed when VWF is subjected to shear stress. Under static conditions, shear stress is mimicked by ristocetin, which in turn triggers a reduction in VWF multimer size upon addition to human plasma.

To evaluate the effect of monovalent (one-arm) anti-VWF CK domain antibodies on the ability of ADAMTS13 to process VWF, human plasma from three healthy donors (stabilized in 3.2% sodium citrate) was incubated overnight at 37° C. in a 10 mM HEPES and 6.5 mM BaCl2 buffer (pH 7.4) with or without 1.5 mg/mL ristocetin and 0 nM or 600 nM of antibody.

The VWF multimers were analyzed via SDS gel electrophoresis. The electrophoretic separation of multimers was performed after dilution of plasma at 1/6 or 1/20 in a dilution buffer supplied by the kit. The plasma samples were electrophoresed on a gel containing a high concentration of agarose (2%) allowing for the separation of VWF multimers in the size range of 449-20000 kDa. VWF multimers were immunofixated with a polyclonal anti-VWF IgG antibody for 1 h followed by a second immunofixation with peroxidase-conjugated anti-IgG antibody for 30 min. The gels were subsequently stained with a peroxidase and chromogenic substrate and prepared for interpretation. Gels were scanned and densitometrically evaluated and quantified relative to the control with peaks 1-3 classified as low molecular weight (LMW) VWF, peaks 4-8 as intermediate molecular weight (IMW) VWF, and all other peaks being high molecular weight (HMW) VWF.

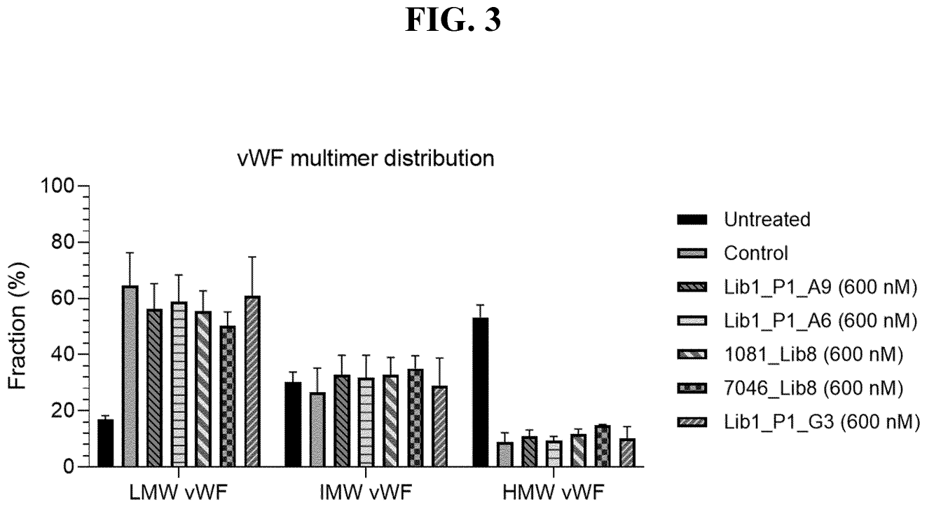

The fractional content of LMW, IMW, and HMW VWF in plasma for the different conditions tested is shown in FIG. 3. Upon treatment of plasma with ristocetin a marked decrease in HMW and corresponding increase in LMW was observed. The same shift in VWF multimer pattern was observed when plasma was treated with ristocetin in the presence of 600 nM antibody including Lib1_P1_A9 and Lib1_P1_A6. These results indicate that the processing of VWF by ADAMTS13 is retained when anti-VWF CK antibodies are bound to the VWF CK domain.

### Example 13: Effect of Anti-VWF CK Domain Antibodies on the Binding of VWF to Collagen

Multimeric VWF binds to collagen in the perivascular connective tissue and in the subendothelial matrix. The A3 domain contains the major binding site for collagen type III). The effect of monovalent (one-arm) anti-VWF CK domain antibodies on the ability of VWF to bind collagen type III was explored by incubating plasma (stabilized in 3.2% sodium citrate) from five healthy human donors for 17 hrs at 37° C. in the presence of 0 nM (“untreated”) or 600 nM antibody. Following incubation, collagen binding was determined relative to untreated plasma. In this assay, VWF in plasma was captured by collagen type III from human placenta coated in the ELISA plate. Next, bound VWF was quantified using a secondary peroxidase-conjugated rabbit anti-VWF antibody.

Relative levels of VWF binding to collagen in presence of antibody are provided in Table 9 and demonstrate that VWF retained the ability to bind collagen in presence of all tested antibodies.

TABLE 9
Binding of VWF in plasma to collagen type III
in the presence one-arm anti-VWF CK
domain antibodies

| | | Collagen |
|---|---|---|
| | Concentration | binding |
| Antibody ID | (nM) | (% control) |
| Lib1_P1_A9 OA | 600 | 106.4 ± 26.9 |
| Lib1_P1_A6 OA | 600 | 103 ± 19.9 |
| 1081_Lib8 | 600 | 107.5 ± 9.1 |
| 7046_Lib8 | 600 | 115.6 ± 26.9 |
| Lib1_P1_G3 | 600 | 108.5 ± 17.1 |

Measured binding levels were normalized to that of untreated plasma from the same donor. Results are shown as mean±standard deviation.

### Example 14: Effect of Anti-VWF CK Domain Antibodies on the VWF:Platelet Interaction

The effect of monovalent (one-arm) anti-VWF CK domain antibodies on binding of VWF to platelets was assessed by incubating platelet rich plasma (PRP) with antibody and ristocetin to facilitate gp1b binding followed by FACS-based detection of VWF binding to platelets.

Blood from healthy donors was collected aseptically into vacuum tubes via an antecubital puncture (1 volume trisodium citrate 0.105 M to 9 volumes blood). PRP was prepared by centrifuging the blood at 250 g for 15 min. PRP from two healthy human donors was diluted 1/4 in HBS buffer (10 mM HEPES, 150 mM NaCl, 1 mM MgSO₄, 5 mM KCl, pH 7.4) and 5 μL was added to a 20-1 reaction mixture consisting of FITC-conjugated anti-VWF antibody (final concentration 8 g/mL), 0.5 mg/ml ristocetin, and 0 or 600 nM monovalent (one-arm) anti-VWF CK domain antibody in HBS-buffer. Samples were incubated for exactly 20 min before quenching by addition of 200 μL of fixation solution (137 mM NaCl, 2.7 mM KCl, 1.12 mM NaH₂PO₄, 1.15 mM KH₂PO₄, 10.2 mM Na₂HPO₄, 4 mM EDTA, 0.5% formaldehyde). Sample analysis was performed on a flow cytometer. Using forward (FSC) and sideward (SSC) scatter pattern, a gate was placed on the platelets. The fluorescent intensity in the FITC gate was selected to determine the binding of VWF to platelets.

The fraction of platelet bound VWF in presence of antibody relative to the same plasma without antibody is listed for each antibody in Table 10. The results show that vWF retain ability to bind platelets in presence of the tested antibodies.

The fraction of platelet bound VWF in presence of antibody relative to the same plasma without antibody is listed for each antibody in Table 10. The results show that vWF retain ability to bind platelets in presence of the tested antibodies.

TABLE 10
Binding of VWF in platelet rich plasma to platelets
in the presence of one-arm anti-VWF CK domain
antibodies and ristocetin

| | | Platelet |
|---|---|---|
| | Concentration | binding |
| Antibody ID | (nM) | (% control) |
| Lib1_P1_A9 OA | 600 | 89.7 ± 4.0 |
| Lib1_P1_A6 OA | 600 | 69.2 ± 4.2 |
| 1081_Lib8 | 600 | 80.3 ± 10.1 |
| 7046_Lib8 | 600 | 78.5 ± 0.8 |
| Lib1_P1 G3 | 600 | 101.3 ± 14.0 |

Measured binding levels were normalized to that of untreated plasma from the same donor. Results are shown as mean±standard deviation.

### Example 15: Quantification of VWF Antigen in Cynomolgus Monkey Plasma Samples

An assay based on the change in turbidity of a microparticle suspension, as measured by photometry, was used to quantify VWF antigen (VWF:Ag) in plasma samples from cynos. A suspension of latex microparticles, coated with antibody specific for VWF, is mixed with the test plasma to be quantified. An antigen-antibody reaction takes place, leading to an agglutination of the latex microparticles which induces an increase in turbidity of the reaction medium. This increase in turbidity is reflected by an increase in absorbance, the latter being measured photometrically. The increase in absorbance is a function of the VWF antigen level present in the test sample.

Plasma levels of VWF were measured in cyno plasma stabilized with 3.2% sodium citrate and diluted 4-fold in 10 mM HEPES, 150 mM NaCl, 1 mM MgSO₄, 5 mM KCl, pH 7.4 buffer.

### Example 16: Quantification of VWF Ristocetin Cofactor Activity (VWF:RCo) in Cynomolgus Monkey Plasma Samples

An assay based on the change in turbidity of a platelet suspension, as measured by photometry, was performed to quantify VWF in plasma samples from cynos. A suspension of reconstituted lyophilized platelets was added to the test plasma followed by the addition of ristocetin to induce VWF- and gp1b-dependent platelet aggregation. Aggregation induces a decrease in turbidity and consequently a decrease in absorbance, which can be measured photometrically. The decrease in absorbance is a function of the VWF activity in the plasma sample.

Plasma activity levels of VWF were measured in cyno plasma stabilized with 3.2% sodium citrate and diluted 4-fold in 10 mM HEPES, 150 mM NaCl, 1 mM MgSO₄, 5 mM KCl, pH 7.4 buffer.

### Example 17: Measurement of VWF Multimer Distribution in Cynomolgus Monkey Plasma Samples

The multimer pattern of VWF in plasma samples from cynos was analyzed as described for human plasma in Example 12. Before analysis, plasma samples were diluted 1:6 or 1:20.

### Example 18: Quantification of FVIII Antigen (FVIII:Ag) in Cynomolgus Monkey Plasma Samples

FVIII antigen (FVIII:Ag) was quantified in plasma samples from cynos. In this assay, FVIII in plasma samples is captured by mouse monoclonal anti-human FVIII:Ag antibody coated on the internal walls of a plastic microplate well. Next, mouse anti-human FVIII antibodies coupled with peroxidase bind to the remaining free antigenic determinants of the bound factor VIII. The bound enzyme peroxidase is revealed by its action on the TMB substrate. After stopping the reaction with a strong acid, the intensity of the color is proportional to the concentration of factor VIII initially present in the sample.

### Example 19: Quantification of Anti-VWF CK Antibody

Quantification of anti-VWF CK domain antibody in plasma samples is done by immunoaffinity capture followed by tryptic digest and quantification using LC-MS/MS.

Using biotinylated goat-anti-human IgG antibody coupled to streptavidin-coated high-capacity magnetic beads, anti-VWF CK domain antibody is selectively captured from cyno plasma samples and combined with an antibody standard. Following rounds of washing of the beads to remove non-specific plasma constituents, bound antibody is subjected to tryptic digest and then quantified by LC-MS/MS. For the quantification, the isotope labelled tryptic peptide VVSVLTVLHQDWLNGK is used. This peptide is derived from the antibody heavy chain and is conserved across IgG1-4 including the antibodies tested in Example 20.

### Example 20: Pharmacokinetics and Pharmacodynamics of Anti-VWF CK Antibodies in Cynomolgus Monkey

The pharmacokinetics of the monovalent (one-arm) Lib1_P1_A9 and Lib1_P1_A6 antibodies and their ability to accumulate VWF and FVIII in circulation was assessed in cynos (n=2 per antibody). Antibodies were administered subcutaneously at day 1 and 8 at 4 mg/kg in a volume of 0.17 ml/kg.

Blood samples were collected at day 1 (pre-dose), 2, 3, 4, 6, 8 (pre-dose), 9, 11, 13, 15 and 22 into polypropylene test tubes with 3.2% sodium citrate as anticoagulant. Platelet poor plasma was prepared by two rounds of centrifugation (2840 g for 10 min) followed by aliquoting into fresh polypropylene tubes and immediate freezing on dry ice. Samples were stored at −80° C. until measurement of VWF:Ag, VWF:RCo, VWF multimer distribution, FVIII:Ag, and antibody levels according to the methods described in Examples 15, 16, 17, 18, and 19, respectively.

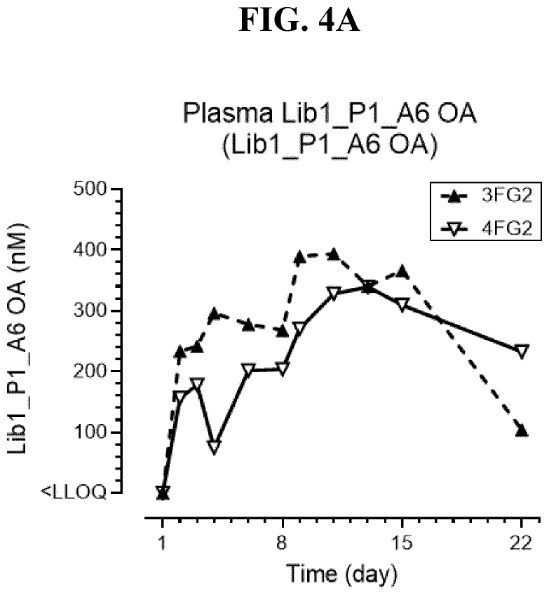

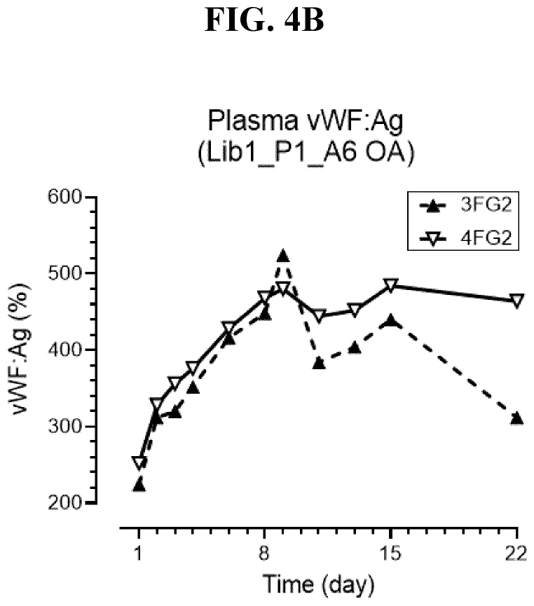

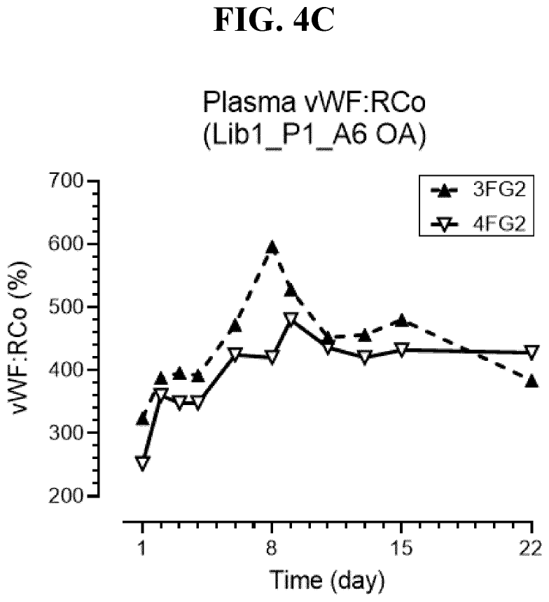

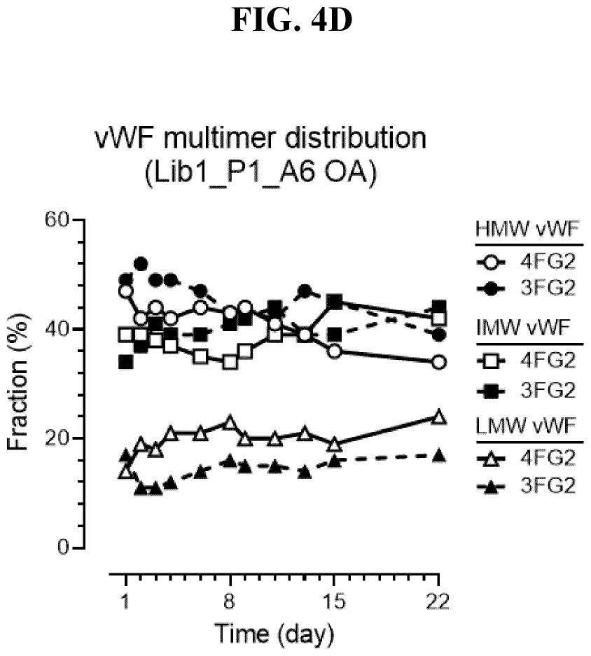

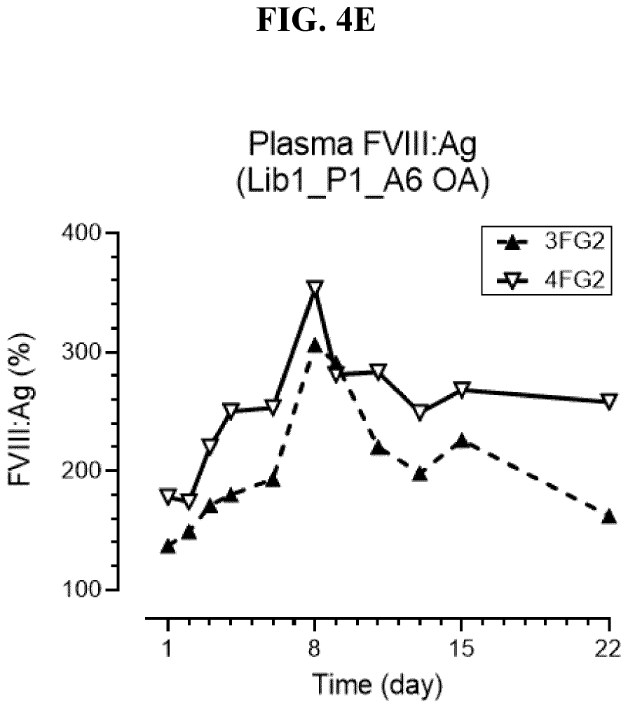

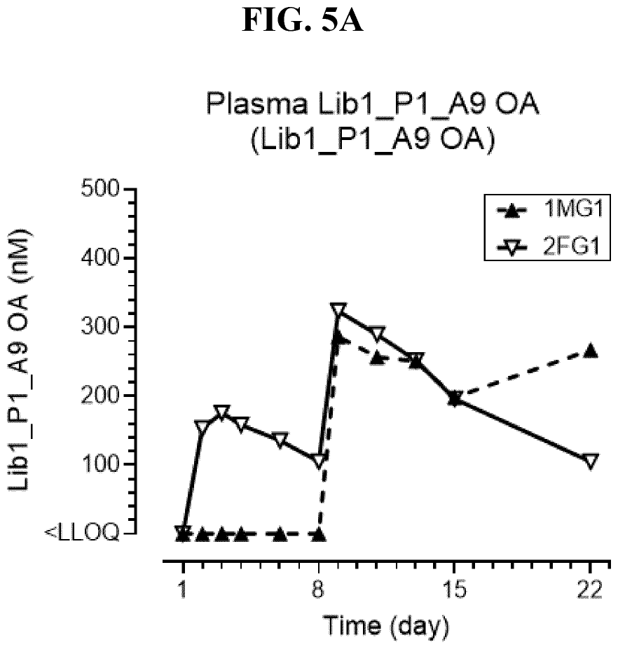

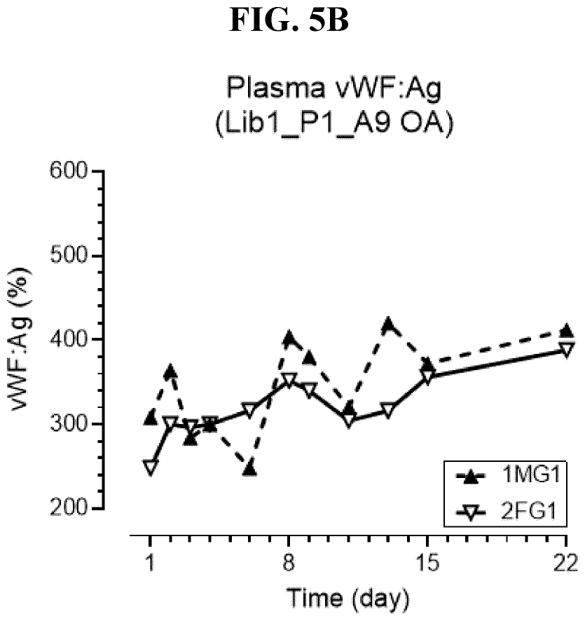

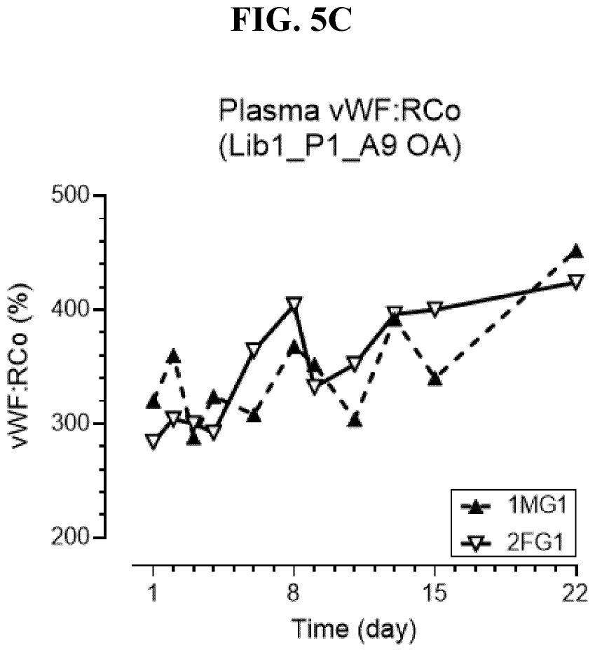

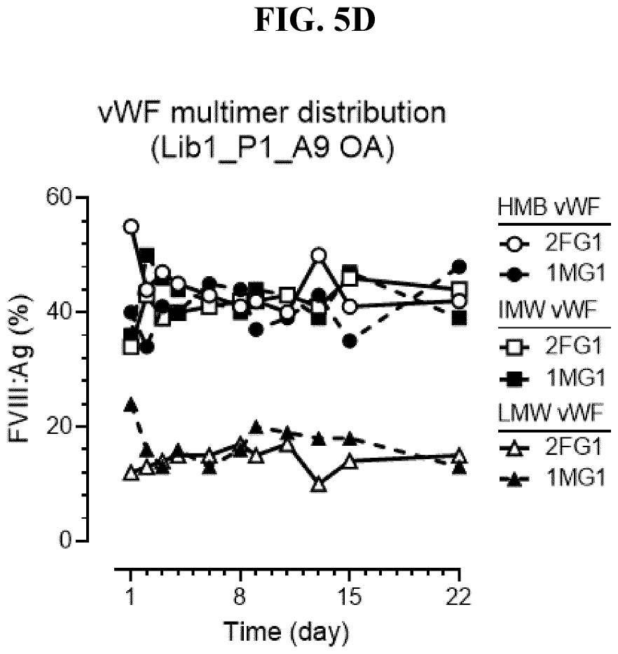

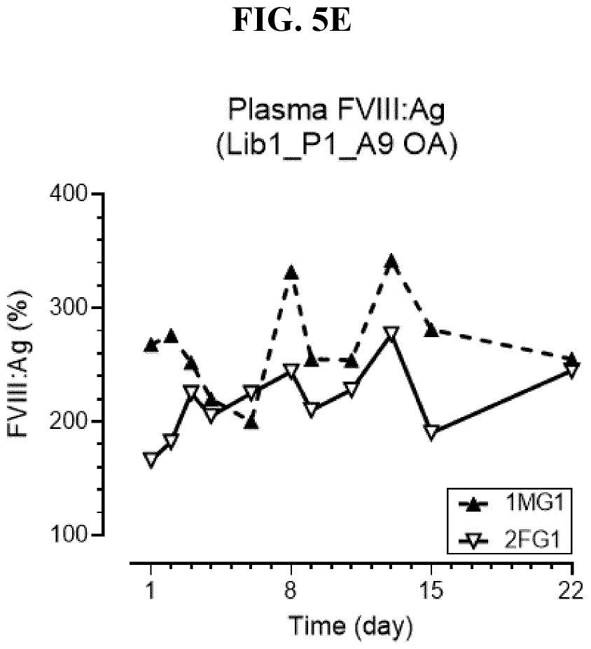

Results for Lib1_P1_A6 OA and Lib1_P1_A9 OA are shown in FIG. 4 and FIG. 5, respectively, and summarized in Table 11. Using the assays with human plasma calibrators, the pre-dose levels of VWF and FVIII were found to be elevated in cynos as compared to humans with mean values of 258% (VWF:Ag), 295% (VWF:RCo), and 187% (FVIII:Ag). From the time-dependent change in VWF and FVIII following dosing of antibody, it can be concluded that both were capable of accumulating VWF. The relative VWF accumulation according to the VWF:RCo assay was comparable to that measured using the VWF:Ag assay, indicating that VWF retains activity upon accumulation and that VWF maintains activity upon anti-VWF CK domain antibody binding, which is in agreement with the functional studies. Consistent with retained FVIII binding by VWF in presence of antibody, accumulation of VWF was further accompanied by a corresponding increase in plasma levels of FVIII. VWF accumulation was most pronounced for the Lib1_P1_A6 OA antibody for which the Cₘₐₓ/Cₚᵣₑ-dose ratio was about 2 and maintained until study end at day 22 in animal 4FG2. In animal 3FG2, levels of Lib1_P1_A6 OA, VWF, and FVIII declined from around day 10. This timing is consistent with anti-drug antibody (ADA) development, which was not addressed in the present study. Analysis of VWF multimers demonstrated a steady distribution of low (LMW), intermediate (IMW) and high (HMW) molecular weight VWF throughout the study comparable to the pre-dose distribution.

TABLE 11
Summary of results from PK/PD study of Lib1_P1_A6 OA
and Lib_P1_A9 OA in cynomolgus monkeys

| | Animal | | C_{pre-dose} | Cₘₐₓ | C_{day22} |
|---|---|---|---|---|---|
| Antibody ID | ID | Assay | (%) | (%) | (%) |
| Lib1_P1_A6 OA | 3FG2 | VWF:Ag | 224 | 524 [2.3] | 312 |
| | | VWF:RCo | 324 | 596 [1.8] | 384 |
| | | FVIII:Ag | 137 | 306 [2.2] | 162 |
| | 4FG2 | VWF:Ag | 252 | 484 [1.9] | 464 |
| | | VWF:RCo | 252 | 480 [1.9] | 428 |
| | | FVIII:Ag | 178 | 353 [2.0] | 258 |
| Lib1_P1_A9 OA | 1MG1 | VWF:Ag | 308 | 420 [1.4] | 412 |
| | | VWF:RCo | 320 | 452 [1.4] | 452 |
| | | FVIII:Ag | 268 | 342 [1.3] | 255 |
| | 2FG1 | VWF:Ag | 248 | 388 [1.6] | 388 |
| | | VWF:RCO | 284 | 424 [1.5] | 424 |
| | | FVIII:Ag | 166 | 277 [1.7] | 245 |

Measured plasma levels (in % of assay standard) of VWF antigen (VWF:Ag), VWF ristocetin cofactor activity (VWF:RCo) and FVIII antigen (FVIII:Ag) before first dosing (C_{pre-dose}), at maximum (Cₘₐₓ), and at day 22 (C_{day22}). The fold accumulation at Cₘₐₓ relative to C_{pre-dose} is given in square brackets.

### Example 21: Administration of an Anti-VWF Antibody to a Subject Having a Blood Disorder Characterized by Excessive Bleeding

Antibodies targeting a CK domain of a human (SEQ ID NO: 5) or a non-human primate (NHP; SEQ ID NO: 6) VWF protein are administered to a subject (e.g., a human) having a blood disorder characterized by excessive bleeding, such as, e.g., von Willebrand disease (VWD). The VWD is congenital VWD. Prior to administration, the subject is, assessed for the presence and/or severity of one or more symptoms selected from epistaxis, cutaneous bleeding, bleeding from minor wounds, oral-cavity bleeding, angiodysplasia, gastrointestinal bleeding, bleeding from tooth extraction, postoperative bleeding, heavy menstrual bleeding, obstetric hemorrhage, hematuria, muscle hematoma, joint bleeding, visceral bleeding, and central nervous system (CNS) bleeding in the subject using routine and conventional methods. The therapeutic efficacy of the pharmaceutical composition in treating the blood disorder is ascertained by measuring VWF activity in blood using bioanalytical assays known to one of skill in the art. Subsequent doses are administered as needed.

### Other Embodiments

Various modifications and variations of the described disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure that are obvious to those skilled in the art are intended to be within the scope of the disclosure. Other embodiments are in the claims.

What is claimed is:

1. A monospecific antibody comprising an antigen-binding site that specifically binds to an epitope of a von Willebrand Factor (VWF) protein positioned at a cysteine knot (CK) domain and a fragment crystallizable (Fc) region, wherein binding of the antibody to the VWF protein in blood plasma results in accumulation of the VWF protein in blood plasma as compared to a blood plasma level of the VWF protein in the absence of the antibody.

2. The monospecific antibody of claim 1, wherein the antibody is a monovalent antibody.

3. The monospecific antibody of claim 1 or 2, wherein the Fc region is a wild-type Fc region.

4. The monospecific antibody of claim 1 or 2, wherein the Fc region is a modified Fc region.

5. The monospecific antibody of claim 4, wherein the modified Fc region comprises one or more mutations selected from the group consisting of a R435H mutation, N434A mutation, T252L/T253S/T254F mutation, E294delta/T307P/N434Y mutation, T256N/A378V/S383N/N434Y mutation, E294 delta mutation, M252Y/S254T/T256E mutation, M428L/N434S mutation, T307A/E380A/N434A mutation, T250Q/M428L mutation, T250Q/M428F mutation, T250E/M428F mutation, T250E/M428L mutation, T256D/Q311V/A378V mutation, T256D/H286D/T307R/Q311V/A378V mutation, H285N/T307Q/N315D mutation, T307Q/Q311V/A378V mutation, H285D/T307Q/A378V mutation, L234F/L235E/P331S mutation, L234F/L235Q/K322Q mutation, L234F/L235Q/P331G mutation, L234F/L235A/K322Q mutation, S228P/F234A/L235A/G237A/P238S mutation, L234A/L235E/G237A/A330S/P331S mutation, F243A/V264A mutation, S228P/L235E/P329G mutation, M252Y/M428L mutation, D2591/V308F mutation, T307Q/N434S mutation, M428L/V308F mutation, Q311V/N434S mutation, H433K/N434F mutation, E258F/V427T mutation, K288E/H435K mutation, F234A/L235A mutation, F234A/L235A/S228P mutation, L234A/L235A/P329G mutation, F234A/L235A/P329G mutation, P329G mutation, F234 mutation, S228P mutation, delta-K447 mutation, L235A mutation, L235E mutation, V308P mutation, V308W mutation, V308Y mutation, V308F mutation, N434S mutation, T307 mutation, Q312A mutation, K322A mutation, P329A mutation, P331A mutation, P238A mutation, D265A mutation, D269A mutation, D270A mutation, N297A mutation, A327Q mutation, P329A mutation, S239A mutation, E294A mutation, Q295A mutation, V303A mutation, A330R mutation, T299A mutation, F234A mutation, T366W mutation, T366S mutation, L368A mutation, Y407V mutation, F405L mutation, K409R mutation, F405L/R409K mutation, and any combination thereof.

6. The monospecific antibody of any one of claims 1-5, wherein the modified Fc region is N-terminally truncated.

7. The monospecific antibody of claim 6, wherein the modified Fc region is N-terminally truncated by 1, 2, 3, 4, 5, 6, 7, 8, 9 10, 11, 12, 13, 14, 16, 17, 18, 19, 20, 21, 22, 23, 24, 25, 26, 27, 28, 29, 30, 31, 32, 33, 34, 35, 36, 37, 38, 39, 40, 41, 42, 43, 44, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more amino acid residues.

8. The monospecific antibody of any one of claims 1-7, wherein the antibody is a human IgG4 antibody comprising a first heavy chain and a second heavy chain.

9. The monospecific antibody of claim 8, wherein the first heavy chain comprises a S228P/T366W/F234A/L235A/delta-K447 mutation and the second heavy chain comprises a S228P/T366S/L368A/Y407V/F234A/L235A/delta-K447 mutation.

10. The monospecific antibody of claim 8, wherein the first heavy chain comprises a S228P/T366W/delta-K447 mutation and the second heavy chain comprises a S228P/T366S/L368A/Y407V/delta-K447 mutation.

11. The monospecific antibody of claim 9 or 10, wherein the first heavy chain and the second heavy chain further comprise a M252Y/S254T/T256E mutation.

12. The monospecific antibody of any one of claims 1-11, wherein the antibody comprises a variable heavy chain (VH) region having an amino acid sequence with at least 80% identity to SEQ ID NO: 12 and a variable light chain (VL) region having an amino acid sequence with at least 80% identity to SEQ ID NO: 16.

13. The monospecific antibody of any one of claims 1-12, wherein the antibody comprises a variable heavy chain (VH) region having an amino acid sequence with at least 85% identity to SEQ ID NO: 12 and a variable light chain (VL) region having an amino acid sequence with at least 85% identity to SEQ ID NO: 16.

14. The monospecific antibody of any one of claims 1-13, wherein the antibody comprises a variable heavy chain (VH) region having an amino acid sequence with at least 90% identity to SEQ ID NO: 12 and a variable light chain (VL) region having an amino acid sequence with at least 90% identity to SEQ ID NO: 16.

15. The monospecific antibody of any one of claims 1-14, wherein the antibody comprises a variable heavy chain (VH) region having an amino acid sequence with at least 95% identity to SEQ ID NO: 12 and a variable light chain (VL) region having an amino acid sequence with at least 95% identity to SEQ ID NO: 16.

16. The monospecific antibody of any one of claims 1-15, wherein the antibody comprises a variable heavy chain (VH) region having an amino acid sequence 100% identical to the amino acid sequence of SEQ ID NO: 12 and a variable light chain (VL) region having an amino acid sequence 100% identical to the amino acid sequence of SEQ ID NO: 16.

17. The monospecific antibody of any one of claims 1-16, wherein the antibody comprises a VH comprising therein a complementarity determining region (CDR) H1 having an amino acid sequence 100% identical to the amino acid sequence of SEQ ID NO: 13, a CDR H2 having an amino acid sequence 100% identical to the amino acid sequence of SEQ ID NO: 14, and a CDR H3 having an amino acid sequence 100% identical to the amino acid sequence of SEQ ID NO: 15; and a VL comprising therein a complementarity determining region (CDR) L1 having an amino acid sequence 100% identical to the amino acid sequence of SEQ ID NO: 17, a CDR L2 having an amino acid sequence 100% identical to the amino acid sequence of KAS, and a CDR L3 having an amino acid sequence 100% identical to the amino acid sequence of SEQ ID NO: 18.

18. The monospecific antibody of any one of claims 1-17, wherein the antibody comprises: (a) a variable heavy chain (VH) region having an amino acid sequence with at least 80% identity to SEQ ID NO: 12 and comprising therein a complementarity determining region (CDR) H1 having an amino acid sequence 100% identical to the amino acid sequence of SEQ ID NO: 13, a CDR H2 having an amino acid sequence 100% identical to the amino acid sequence of SEQ ID NO: 14, and a CDR H3 having an amino acid sequence 100% identical to the amino acid sequence of SEQ ID NO: 15; a variable light chain (VL) region having an amino acid sequence with at least 80% identity to SEQ ID NO: 16 and comprising therein a complementarity determining region (CDR) L1 having an amino acid sequence 100% identical to the amino acid sequence of SEQ ID NO: 17, a CDR L2 having an amino acid sequence 100% identical to the amino acid sequence of KAS, and a CDR L3 having an amino acid sequence 100% identical to the amino acid sequence of SEQ ID NO: 18.

19. The monospecific antibody of any one of claims 1-18, wherein the antibody comprises a first heavy chain (HC) region comprising an amino acid sequence having at least 80% identity to SEQ ID NO: 453, a second heavy chain region comprising an amino acid sequence having at least 80% identity to SEQ ID NO: 473, and a light chain (LC) region, comprising an amino acid sequence having at least 80% identity to SEQ ID NO: 463.

20. The monospecific antibody of any one of claims 1-11, wherein the antibody comprises a variable heavy chain (VH) region having an amino acid sequence with at least 80% identity to SEQ ID NO: 19 and a variable light chain (VL) region having an amino acid sequence with at least 80% identity to SEQ ID NO: 23.

21. The monospecific antibody of any one of claims 1-11 and 20, wherein the antibody comprises a variable heavy chain (VH) region having an amino acid sequence with at least 85% identity to SEQ ID NO: 19 and a variable light chain (VL) region having an amino acid sequence with at least 85% identity to SEQ ID NO: 23.

22. The monospecific antibody of any one of claims 1-11, 20, and 21, wherein the antibody comprises a variable heavy chain (VH) region having an amino acid sequence with at least 90% identity to SEQ ID NO: 19 and a variable light chain (VL) region having an amino acid sequence with at least 90% identity to SEQ ID NO: 23.

23. The monospecific antibody of any one of claims 1-11 and 20-22, wherein the antibody comprises a variable heavy chain (VH) region having an amino acid sequence with at least 95% identity to SEQ ID NO: 19 and a variable light chain (VL) region having an amino acid sequence with at least 95% identity to SEQ ID NO: 23.

24. The monospecific antibody of any one of claims 1-11 and 20-23, wherein the antibody comprises a variable heavy chain (VH) region having an amino acid sequence 100% identical to the amino acid sequence of SEQ ID NO: 19 and a variable light chain (VL) region having an amino acid sequence 100% identical to the amino acid sequence of SEQ ID NO: 23.

25. The monospecific antibody of any one of claims 1-11 and 20-24, wherein the antibody comprises a VH comprising therein a complementarity determining region (CDR) H1 having an amino acid sequence 100% identical to the amino acid sequence of SEQ ID NO: 20, a CDR H2 having an amino acid sequence 100% identical to the amino acid sequence of SEQ ID NO: 21, and a CDR H3 having an amino acid sequence 100% identical to the amino acid sequence of SEQ ID NO: 22; and a VL comprising therein a complementarity determining region (CDR) L1 having an amino acid sequence 100% identical to the amino acid sequence of SEQ ID NO: 24, a CDR L2 having an amino acid sequence 100% identical to the amino acid sequence of AAS, and a CDR L3 having an amino acid sequence 100% identical to the amino acid sequence of SEQ ID NO: 25.

26. The monospecific antibody of any one of claims 1-11 and 20-25, wherein the antibody comprises: (a) a variable heavy chain (VH) region having an amino acid sequence with at least 80% identity to SEQ ID NO: 19 and comprising therein a complementarity determining region (CDR) H1 having an amino acid sequence 100% identical to the amino acid sequence of SEQ ID NO: 20, a CDR H2 having an amino acid sequence 100% identical to the amino acid sequence of SEQ ID NO: 21, and a CDR H3 having an amino acid sequence 100% identical to the amino acid sequence of SEQ ID NO: 22; a variable light chain (VL) region having an amino acid sequence with at least 80% identity to SEQ ID NO: 23 and comprising therein a complementarity determining region (CDR) L1 having an amino acid sequence 100% identical to the amino acid sequence of SEQ ID NO: 24, a CDR L2 having an amino acid sequence 100% identical to the amino acid sequence of AAS, and a CDR L3 having an amino acid sequence 100% identical to the amino acid sequence of SEQ ID NO: 25.

27. The monospecific antibody of any one of claims 1-11 and 20-26, wherein the antibody comprises a first heavy chain (HC) region comprising an amino acid sequence having at least 80% identity to SEQ ID NO: 454, a second heavy chain region comprising an amino acid sequence having at least 80% identity to SEQ ID NO: 474, and a light chain (LC) region, comprising an amino acid sequence having at least 80% identity to SEQ ID NO: 464.

28. The monospecific antibody of any one of claims 1-11, wherein the antibody comprises a variable heavy chain (VH) region having an amino acid sequence with at least 80% identity to SEQ ID NO: 26 and a variable light chain (VL) region having an amino acid sequence with at least 80% identity to SEQ ID NO: 30.

29. The monospecific antibody of any one of claims 1-11 and 28, wherein the antibody comprises a variable heavy chain (VH) region having an amino acid sequence with at least 85% identity to SEQ ID NO: 26 and a variable light chain (VL) region having an amino acid sequence with at least 85% identity to SEQ ID NO: 30.

30. The monospecific antibody of any one of claims 1-11, 28, and 29 wherein the antibody comprises a variable heavy chain (VH) region having an amino acid sequence with at least 90% identity to SEQ ID NO: 26 and a variable light chain (VL) region having an amino acid sequence with at least 90% identity to SEQ ID NO: 30.

31. The monospecific antibody of any one of claims 1-11 and 28-30, wherein the antibody comprises a variable heavy chain (VH) region having an amino acid sequence with at least 95% identity to SEQ ID NO: 26 and a variable light chain (VL) region having an amino acid sequence with at least 95% identity to SEQ ID NO: 30.

32. The monospecific antibody of any one of claims 1-11 and 28-31, wherein the antibody comprises a variable heavy chain (VH) region having an amino acid sequence 100% identical to the amino acid sequence of SEQ ID NO: 26 and a variable light chain (VL) region having an amino acid sequence 100% identical to the amino acid sequence of SEQ ID NO: 30.

33. The monospecific antibody of any one of claims 1-11 and 28-32, wherein the antibody comprises a VH comprising therein a complementarity determining region (CDR) H1 having an amino acid sequence 100% identical to the amino acid sequence of SEQ ID NO: 27, a CDR H2 having an amino acid sequence 100% identical to the amino acid sequence of SEQ ID NO: 28, and a CDR H3 having an amino acid sequence 100% identical to the amino acid sequence of SEQ ID NO: 29; and a VL comprising therein a complementarity determining region (CDR) L1 having an amino acid sequence 100% identical to the amino acid sequence of SEQ ID NO: 31, a CDR L2 having an amino acid sequence 100% identical to the amino acid sequence of ESS, and a CDR L3 having an amino acid sequence 100% identical to the amino acid sequence of SEQ ID NO: 32.

34. The monospecific antibody of any one of claims 1-11 and 28-33, wherein the antibody comprises: (a) a variable heavy chain (VH) region having an amino acid sequence with at least 80% identity to SEQ ID NO: 26 and comprising therein a complementarity determining region (CDR) H1 having an amino acid sequence 100% identical to the amino acid sequence of SEQ ID NO: 27, a CDR H2 having an amino acid sequence 100% identical to the amino acid sequence of SEQ ID NO: 28, and a CDR H3 having an amino acid sequence 100% identical to the amino acid sequence of SEQ ID NO: 29; a variable light chain (VL) region having an amino acid sequence with at least 80% identity to SEQ ID NO: 30 and comprising therein a complementarity determining region (CDR) L1 having an amino acid sequence 100% identical to the amino acid sequence of SEQ ID NO: 31, a CDR L2 having an amino acid sequence 100% identical to the amino acid sequence of ESS, and a CDR L3 having an amino acid sequence 100% identical to the amino acid sequence of SEQ ID NO: 32.

35. The monospecific antibody of any one of claims 1-11, wherein the antibody comprises a variable heavy chain (VH) region having an amino acid sequence with at least 80% identity to SEQ ID NO: 33 and a variable light chain (VL) region having an amino acid sequence with at least 80% identity to SEQ ID NO: 37.

36. The monospecific antibody of any one of claims 1-11 and 35, wherein the antibody comprises a variable heavy chain (VH) region having an amino acid sequence with at least 85% identity to SEQ ID NO: 33 and a variable light chain (VL) region having an amino acid sequence with at least 85% identity to SEQ ID NO: 37.

37. The monospecific antibody of any one of claims 1-11, 35, and 36, wherein the antibody comprises a variable heavy chain (VH) region having an amino acid sequence with at least 90% identity to SEQ ID NO: 33 and a variable light chain (VL) region having an amino acid sequence with at least 90% identity to SEQ ID NO: 37.

38. The monospecific antibody of any one of claims 1-11 and 35-37, wherein the antibody comprises a variable heavy chain (VH) region having an amino acid sequence with at least 95% identity to SEQ ID NO: 33 and a variable light chain (VL) region having an amino acid sequence with at least 95% identity to SEQ ID NO: 37.

39. The monospecific antibody of any one of claims 1-11 and 35-38, wherein the antibody comprises a variable heavy chain (VH) region having an amino acid sequence 100% identical to the amino acid sequence of SEQ ID NO: 33 and a variable light chain (VL) region having an amino acid sequence 100% identical to the amino acid sequence of SEQ ID NO: 37.

40. The monospecific antibody of any one of claims 1-11 and 35-39, wherein the antibody comprises a VH comprising therein a complementarity determining region (CDR) H1 having an amino acid sequence 100% identical to the amino acid sequence of SEQ ID NO: 34, a CDR H2 having an amino acid sequence 100% identical to the amino acid sequence of SEQ ID NO: 35, and a CDR H3 having an amino acid sequence 100% identical to the amino acid sequence of SEQ ID NO: 36; and a VL comprising therein a complementarity determining region (CDR) L1 having an amino acid sequence 100% identical to the amino acid sequence of SEQ ID NO: 38, a CDR L2 having an amino acid sequence 100% identical to the amino acid sequence of DAT, and a CDR L3 having an amino acid sequence 100% identical to the amino acid sequence of SEQ ID NO: 39.

41. The monospecific antibody of any one of claims 1-11 and 35-40, wherein the antibody comprises: (a) a variable heavy chain (VH) region having an amino acid sequence with at least 80% identity to SEQ ID NO: 33 and comprising therein a complementarity determining region (CDR) H1 having an amino acid sequence 100% identical to the amino acid sequence of SEQ ID NO: 34, a CDR H2 having an amino acid sequence 100% identical to the amino acid sequence of SEQ ID NO: 35, and a CDR H3 having an amino acid sequence 100% identical to the amino acid sequence of SEQ ID NO: 36; a variable light chain (VL) region having an amino acid sequence with at least 80% identity to SEQ ID NO: 37 and comprising therein a complementarity determining region (CDR) L1 having an amino acid sequence 100% identical to the amino acid sequence of SEQ ID NO: 38, a CDR L2 having an amino acid sequence 100% identical to the amino acid sequence of DAT, and a CDR L3 having an amino acid sequence 100% identical to the amino acid sequence of SEQ ID NO: 39.

42. The monospecific antibody of any one of claims 1-11 and 35-41, wherein the antibody comprises a first heavy chain (HC) region comprising an amino acid sequence having at least 80% identity to SEQ ID NO: 455, a second heavy chain region comprising an amino acid sequence having at least 80% identity to SEQ ID NO: 475, and a light chain (LC) region, comprising an amino acid sequence having at least 80% identity to SEQ ID NO: 465.

43. The monospecific antibody of any one of claims 1-11, wherein the antibody comprises a variable heavy chain (VH) region having an amino acid sequence with at least 80% identity to SEQ ID NO: 40 and a variable light chain (VL) region having an amino acid sequence with at least 80% identity to SEQ ID NO: 44.

44. The monospecific antibody of any one of claims 1-11 and 43, wherein the antibody comprises a variable heavy chain (VH) region having an amino acid sequence with at least 85% identity to SEQ ID NO: 40 and a variable light chain (VL) region having an amino acid sequence with at least 85% identity to SEQ ID NO: 44.

45. The monospecific antibody of any one of claims 1-11, 43, and 44, wherein the antibody comprises a variable heavy chain (VH) region having an amino acid sequence with at least 90% identity to SEQ ID NO: 40 and a variable light chain (VL) region having an amino acid sequence with at least 90% identity to SEQ ID NO: 44.

46. The monospecific antibody of any one of claims 1-11 and 43-45, wherein the antibody comprises a variable heavy chain (VH) region having an amino acid sequence with at least 95% identity to SEQ ID NO: 40 and a variable light chain (VL) region having an amino acid sequence with at least 95% identity to SEQ ID NO: 44.

47. The monospecific antibody of any one of claims 1-11 and 43-46, wherein the antibody comprises a variable heavy chain (VH) region having an amino acid sequence 100% identical to the amino acid sequence of SEQ ID NO: 40 and a variable light chain (VL) region having an amino acid sequence 100% identical to the amino acid sequence of SEQ ID NO: 44.

48. The monospecific antibody of any one of claims 1-11 and 43-47, wherein the antibody comprises a VH comprising therein a complementarity determining region (CDR) H1 having an amino acid sequence 100% identical to the amino acid sequence of SEQ ID NO: 41, a CDR H2 having an amino acid sequence 100% identical to the amino acid sequence of SEQ ID NO: 42, and a CDR H3 having an amino acid sequence 100% identical to the amino acid sequence of SEQ ID NO: 43; and a VL comprising therein a complementarity determining region (CDR) L1 having an amino acid sequence 100% identical to the amino acid sequence of SEQ ID NO: 45, a CDR L2 having an amino acid sequence 100% identical to the amino acid sequence of GSS, and a CDR L3 having an amino acid sequence 100% identical to the amino acid sequence of SEQ ID NO: 46.

49. The monospecific antibody of any one of claims 1-11 and 43-48, wherein the antibody comprises: (a) a variable heavy chain (VH) region having an amino acid sequence with at least 80% identity to SEQ ID NO: 40 and comprising therein a complementarity determining region (CDR) H1 having an amino acid sequence 100% identical to the amino acid sequence of SEQ ID NO: 41, a CDR H2 having an amino acid sequence 100% identical to the amino acid sequence of SEQ ID NO: 42, and a CDR H3 having an amino acid sequence 100% identical to the amino acid sequence of SEQ ID NO: 43; a variable light chain (VL) region having an amino acid sequence with at least 80% identity to SEQ ID NO: 44 and comprising therein a complementarity determining region (CDR) L1 having an amino acid sequence 100% identical to the amino acid sequence of SEQ ID NO: 45, a CDR L2 having an amino acid sequence 100% identical to the amino acid sequence of GSS, and a CDR L3 having an amino acid sequence 100% identical to the amino acid sequence of SEQ ID NO: 46.

50. The monospecific antibody of any one of claims 1-11 and 43-49, wherein the antibody comprises a first heavy chain (HC) region comprising an amino acid sequence having at least 80% identity to SEQ ID NO: 456, a second heavy chain region comprising an amino acid sequence having at least 80% identity to SEQ ID NO: 476, and a light chain (LC) region, comprising an amino acid sequence having at least 80% identity to SEQ ID NO: 466.

51. The monospecific antibody of any one of claims 1-11, wherein the antibody comprises a variable heavy chain (VH) region having an amino acid sequence with at least 80% identity to SEQ ID NO: 47 and a variable light chain (VL) region having an amino acid sequence with at least 80% identity to SEQ ID NO: 51.

52. The monospecific antibody of any one of claims 1-11 and 51, wherein the antibody comprises a variable heavy chain (VH) region having an amino acid sequence with at least 85% identity to SEQ ID NO: 47 and a variable light chain (VL) region having an amino acid sequence with at least 85% identity to SEQ ID NO: 51.

53. The monospecific antibody of any one of claims 1-11, 51, and 52, wherein the antibody comprises a variable heavy chain (VH) region having an amino acid sequence with at least 90% identity to SEQ ID NO: 47 and a variable light chain (VL) region having an amino acid sequence with at least 90% identity to SEQ ID NO: 51.

54. The monospecific antibody of any one of claims 1-11 and 51-53, wherein the antibody comprises a variable heavy chain (VH) region having an amino acid sequence with at least 95% identity to SEQ ID NO: 47 and a variable light chain (VL) region having an amino acid sequence with at least 95% identity to SEQ ID NO: 51.

55. The monospecific antibody of any one of claims 1-11 and 51-54, wherein the antibody comprises a variable heavy chain (VH) region having an amino acid sequence 100% identical to the amino acid sequence of SEQ ID NO: 47 and a variable light chain (VL) region having an amino acid sequence 100% identical to the amino acid sequence of SEQ ID NO: 51.

56. The monospecific antibody of any one of claims 1-11 and 51-55, wherein the antibody comprises a VH comprising therein a complementarity determining region (CDR) H1 having an amino acid sequence 100% identical to the amino acid sequence of SEQ ID NO: 48, a CDR H2 having an amino acid sequence 100% identical to the amino acid sequence of SEQ ID NO: 49, and a CDR H3 having an amino acid sequence 100% identical to the amino acid sequence of SEQ ID NO: 50; and a VL comprising therein a complementarity determining region (CDR) L1 having an amino acid sequence 100% identical to the amino acid sequence of SEQ ID NO: 52, a CDR L2 having an amino acid sequence 100% identical to the amino acid sequence of DAT, and a CDR L3 having an amino acid sequence 100% identical to the amino acid sequence of SEQ ID NO: 53.

57. The monospecific antibody of any one of claims 1-11 and 51-56, wherein the antibody comprises: (a) a variable heavy chain (VH) region having an amino acid sequence with at least 80% identity to SEQ ID NO: 47 and comprising therein a complementarity determining region (CDR) H1 having an amino acid sequence 100% identical to the amino acid sequence of SEQ ID NO: 48, a CDR H2 having an amino acid sequence 100% identical to the amino acid sequence of SEQ ID NO: 49, and a CDR H3 having an amino acid sequence 100% identical to the amino acid sequence of SEQ ID NO: 50; a variable light chain (VL) region having an amino acid sequence with at least 80% identity to SEQ ID NO: 51 and comprising therein a complementarity determining region (CDR) L1 having an amino acid sequence 100% identical to the amino acid sequence of SEQ ID NO: 52, a CDR L2 having an amino acid sequence 100% identical to the amino acid sequence of DAT, and a CDR L3 having an amino acid sequence 100% identical to the amino acid sequence of SEQ ID NO: 53.

58. The monospecific antibody of any one of claims 1-11, wherein the antibody comprises a variable heavy chain (VH) region having an amino acid sequence with at least 80% identity to SEQ ID NO: 54 and a variable light chain (VL) region having an amino acid sequence with at least 80% identity to SEQ ID NO: 58.

59. The monospecific antibody of any one of claims 1-11 and 58, wherein the antibody comprises a variable heavy chain (VH) region having an amino acid sequence with at least 85% identity to SEQ ID NO: 54 and a variable light chain (VL) region having an amino acid sequence with at least 85% identity to SEQ ID NO: 58.

60. The monospecific antibody of any one of claims 1-11, 58, and 59, wherein the antibody comprises a variable heavy chain (VH) region having an amino acid sequence with at least 90% identity to SEQ ID NO: 54 and a variable light chain (VL) region having an amino acid sequence with at least 90% identity to SEQ ID NO: 58.

61. The monospecific antibody of any one of claims 1-11 and 58-60, wherein the antibody comprises a variable heavy chain (VH) region having an amino acid sequence with at least 95% identity to SEQ ID NO: 54 and a variable light chain (VL) region having an amino acid sequence with at least 95% identity to SEQ ID NO: 58.

62. The monospecific antibody of any one of claims 1-11 and 58-61, wherein the antibody comprises a variable heavy chain (VH) region having an amino acid sequence 100% identical to the amino acid sequence of SEQ ID NO: 54 and a variable light chain (VL) region having an amino acid sequence 100% identical to the amino acid sequence of SEQ ID NO: 58.

63. The monospecific antibody of any one of claims 1-11 and 58-62, wherein the antibody comprises a VH comprising therein a complementarity determining region (CDR) H1 having an amino acid sequence 100% identical to the amino acid sequence of SEQ ID NO: 55, a CDR H2 having an amino acid sequence 100% identical to the amino acid sequence of SEQ ID NO: 56, and a CDR H3 having an amino acid sequence 100% identical to the amino acid sequence of SEQ ID NO: 57; and a VL comprising therein a complementarity determining region (CDR) L1 having an amino acid sequence 100% identical to the amino acid sequence of SEQ ID NO: 59, a CDR L2 having an amino acid sequence 100% identical to the amino acid sequence of GAS, and a CDR L3 having an amino acid sequence 100% identical to the amino acid sequence of SEQ ID NO: 60.

64. The monospecific antibody of any one of claims 1-11 and 58-63, wherein the antibody comprises: (a) a variable heavy chain (VH) region having an amino acid sequence with at least 80% identity to SEQ ID NO: 54 and comprising therein a complementarity determining region (CDR) H1 having an amino acid sequence 100% identical to the amino acid sequence of SEQ ID NO: 55, a CDR H2 having an amino acid sequence 100% identical to the amino acid sequence of SEQ ID NO: 56, and a CDR H3 having an amino acid sequence 100% identical to the amino acid sequence of SEQ ID NO: 57; a variable light chain (VL) region having an amino acid sequence with at least 80% identity to SEQ ID NO: 58 and comprising therein a complementarity determining region (CDR) L1 having an amino acid sequence 100% identical to the amino acid sequence of SEQ ID NO: 59, a CDR L2 having an amino acid sequence 100% identical to the amino acid sequence of GAS, and a CDR L3 having an amino acid sequence 100% identical to the amino acid sequence of SEQ ID NO: 60.

65. The monospecific antibody of any one of claims 1-11 and 58-64, wherein the antibody comprises a first heavy chain (HC) region comprising an amino acid sequence having at least 80% identity to SEQ ID NO: 457, a second heavy chain region comprising an amino acid sequence having at least 80% identity to SEQ ID NO: 577, and a light chain (LC) region, comprising an amino acid sequence having at least 80% identity to SEQ ID NO: 567.

66. The monospecific antibody of any one of claims 1-11, wherein the antibody comprises a variable heavy chain (VH) region having an amino acid sequence with at least 80% identity to SEQ ID NO: 61 and a variable light chain (VL) region having an amino acid sequence with at least 80% identity to SEQ ID NO: 65.

67. The monospecific antibody of any one of claims 1-11 and 66, wherein the antibody comprises a variable heavy chain (VH) region having an amino acid sequence with at least 85% identity to SEQ ID NO: 61 and a variable light chain (VL) region having an amino acid sequence with at least 85% identity to SEQ ID NO: 65.

68. The monospecific antibody of any one of claims 1-11, 66, and 67, wherein the antibody comprises a variable heavy chain (VH) region having an amino acid sequence with at least 90% identity to SEQ ID NO: 61 and a variable light chain (VL) region having an amino acid sequence with at least 90% identity to SEQ ID NO: 65.

69. The monospecific antibody of any one of claims 1-11 and 66-68, wherein the antibody comprises a variable heavy chain (VH) region having an amino acid sequence with at least 95% identity to SEQ ID NO: 61 and a variable light chain (VL) region having an amino acid sequence with at least 95% identity to SEQ ID NO: 65.

70. The monospecific antibody of any one of claims 1-11 and 66-69, wherein the antibody comprises a variable heavy chain (VH) region having an amino acid sequence 100% identical to the amino acid sequence of SEQ ID NO: 61 and a variable light chain (VL) region having an amino acid sequence 100% identical to the amino acid sequence of SEQ ID NO: 65.

71. The monospecific antibody of any one of claims 1-11 and 66-70, wherein the antibody comprises a VH comprising therein a complementarity determining region (CDR) H1 having an amino acid sequence 100% identical to the amino acid sequence of SEQ ID NO: 62, a CDR H2 having an amino acid sequence 100% identical to the amino acid sequence of SEQ ID NO: 63, and a CDR H3 having an amino acid sequence 100% identical to the amino acid sequence of SEQ ID NO: 64; and a VL comprising therein a complementarity determining region (CDR) L1 having an amino acid sequence 100% identical to the amino acid sequence of SEQ ID NO: 66, a CDR L2 having an amino acid sequence 100% identical to the amino acid sequence of DAS, and a CDR L3 having an amino acid sequence 100% identical to the amino acid sequence of SEQ ID NO: 67.

72. The monospecific antibody of any one of claims 1-11 and 66-71, wherein the antibody comprises: (a) a variable heavy chain (VH) region having an amino acid sequence with at least 80% identity to SEQ ID NO: 61 and comprising therein a complementarity determining region (CDR) H1 having an amino acid sequence 100% identical to the amino acid sequence of SEQ ID NO: 62, a CDR H2 having an amino acid sequence 100% identical to the amino acid sequence of SEQ ID NO: 63, and a CDR H3 having an amino acid sequence 100% identical to the amino acid sequence of SEQ ID NO: 64; a variable light chain (VL) region having an amino acid sequence with at least 80% identity to SEQ ID NO: 65 and comprising therein a complementarity determining region (CDR) L1 having an amino acid sequence 100% identical to the amino acid sequence of SEQ ID NO: 66, a CDR L2 having an amino acid sequence 100% identical to the amino acid sequence of DAS, and a CDR L3 having an amino acid sequence 100% identical to the amino acid sequence of SEQ ID NO: 67.

73. The monospecific antibody of any one of claims 1-11, wherein the antibody comprises a variable heavy chain (VH) region having an amino acid sequence with at least 80% identity to SEQ ID NO: 61 and a variable light chain (VL) region having an amino acid sequence with at least 80% identity to SEQ ID NO: 65.

74. The monospecific antibody of any one of claims 1-11 and 73, wherein the antibody comprises a variable heavy chain (VH) region having an amino acid sequence with at least 85% identity to SEQ ID NO: 61 and a variable light chain (VL) region having an amino acid sequence with at least 85% identity to SEQ ID NO: 65.

75. The monospecific antibody of any one of claims 1-11, 73, and 74, wherein the antibody comprises a variable heavy chain (VH) region having an amino acid sequence with at least 90% identity to SEQ ID NO: 61 and a variable light chain (VL) region having an amino acid sequence with at least 90% identity to SEQ ID NO: 65.

76. The monospecific antibody of any one of claims 1-11 and 73-75, wherein the antibody comprises a variable heavy chain (VH) region having an amino acid sequence with at least 95% identity to SEQ ID NO: 61 and a variable light chain (VL) region having an amino acid sequence with at least 95% identity to SEQ ID NO: 65.

77. The monospecific antibody of any one of claims 1-11 and 73-76, wherein the antibody comprises a variable heavy chain (VH) region having an amino acid sequence 100% identical to the amino acid sequence of SEQ ID NO: 61 and a variable light chain (VL) region having an amino acid sequence 100% identical to the amino acid sequence of SEQ ID NO: 65.

78. The monospecific antibody of any one of claims 1-11 and 73-77, wherein the antibody comprises a VH comprising therein a complementarity determining region (CDR) H1 having an amino acid sequence 100% identical to the amino acid sequence of SEQ ID NO: 62, a CDR H2 having an amino acid sequence 100% identical to the amino acid sequence of SEQ ID NO: 63, and a CDR H3 having an amino acid sequence 100% identical to the amino acid sequence of SEQ ID NO: 64; and a VL comprising therein a complementarity determining region (CDR) L1 having an amino acid sequence 100% identical to the amino acid sequence of SEQ ID NO: 66, a CDR L2 having an amino acid sequence 100% identical to the amino acid sequence of DAS, and a CDR L3 having an amino acid sequence 100% identical to the amino acid sequence of SEQ ID NO: 67.

79. The monospecific antibody of any one of claims 1-11 and 73-78, wherein the antibody comprises: (a) a variable heavy chain (VH) region having an amino acid sequence with at least 80% identity to SEQ ID NO: 61 and comprising therein a complementarity determining region (CDR) H1 having an amino acid sequence 100% identical to the amino acid sequence of SEQ ID NO: 62, a CDR H2 having an amino acid sequence 100% identical to the amino acid sequence of SEQ ID NO: 63, and a CDR H3 having an amino acid sequence 100% identical to the amino acid sequence of SEQ ID NO: 64; a variable light chain (VL) region having an amino acid sequence with at least 80% identity to SEQ ID NO: 65 and comprising therein a complementarity determining region (CDR) L1 having an amino acid sequence 100% identical to the amino acid sequence of SEQ ID NO: 66, a CDR L2 having an amino acid sequence 100% identical to the amino acid sequence of DAS, and a CDR L3 having an amino acid sequence 100% identical to the amino acid sequence of SEQ ID NO: 67.

80. The monospecific antibody of any one of claims 1-11, wherein the antibody comprises a variable heavy chain (VH) region having an amino acid sequence with at least 80% identity to SEQ ID NO: 68 and a variable light chain (VL) region having an amino acid sequence with at least 80% identity to SEQ ID NO: 72.

81. The monospecific antibody of any one of claims 1-11 and 80, wherein the antibody comprises a variable heavy chain (VH) region having an amino acid sequence with at least 85% identity to SEQ ID NO: 68 and a variable light chain (VL) region having an amino acid sequence with at least 85% identity to SEQ ID NO: 72.

82. The monospecific antibody of any one of claims 1-11, 80, and 81, wherein the antibody comprises a variable heavy chain (VH) region having an amino acid sequence with at least 90% identity to SEQ ID NO: 68 and a variable light chain (VL) region having an amino acid sequence with at least 90% identity to SEQ ID NO: 72.

83. The monospecific antibody of any one of claims 1-11 and 80-82, wherein the antibody comprises a variable heavy chain (VH) region having an amino acid sequence with at least 95% identity to SEQ ID NO: 68 and a variable light chain (VL) region having an amino acid sequence with at least 95% identity to SEQ ID NO: 72.

84. The monospecific antibody of any one of claims 1-11 and 80-83, wherein the antibody comprises a variable heavy chain (VH) region having an amino acid sequence 100% identical to the amino acid sequence of SEQ ID NO: 68 and a variable light chain (VL) region having an amino acid sequence 100% identical to the amino acid sequence of SEQ ID NO: 72.

85. The monospecific antibody of any one of claims 1-11 and 80-84, wherein the antibody comprises a VH comprising therein a complementarity determining region (CDR) H1 having an amino acid sequence 100% identical to the amino acid sequence of SEQ ID NO: 69, a CDR H2 having an amino acid sequence 100% identical to the amino acid sequence of SEQ ID NO: 70, and a CDR H3 having an amino acid sequence 100% identical to the amino acid sequence of SEQ ID NO: 71; and a VL comprising therein a complementarity determining region (CDR) L1 having an amino acid sequence 100% identical to the amino acid sequence of SEQ ID NO: 73, a CDR L2 having an amino acid sequence 100% identical to the amino acid sequence of GAS, and a CDR L3 having an amino acid sequence 100% identical to the amino acid sequence of SEQ ID NO: 74.

86. The monospecific antibody of any one of claims 1-11 and 80-85, wherein the antibody comprises: (a) a variable heavy chain (VH) region having an amino acid sequence with at least 80% identity to SEQ ID NO: 68 and comprising therein a complementarity determining region (CDR) H1 having an amino acid sequence 100% identical to the amino acid sequence of SEQ ID NO: 69, a CDR H2 having an amino acid sequence 100% identical to the amino acid sequence of SEQ ID NO: 70, and a CDR H3 having an amino acid sequence 100% identical to the amino acid sequence of SEQ ID NO: 71; a variable light chain (VL) region having an amino acid sequence with at least 80% identity to SEQ ID NO: 72 and comprising therein a complementarity determining region (CDR) L1 having an amino acid sequence 100% identical to the amino acid sequence of SEQ ID NO: 73, a CDR L2 having an amino acid sequence 100% identical to the amino acid sequence of GAS, and a CDR L3 having an amino acid sequence 100% identical to the amino acid sequence of SEQ ID NO: 74.

87. The monospecific antibody of any one of claims 1-11, wherein the antibody comprises a variable heavy chain (VH) region having an amino acid sequence with at least 80% identity to SEQ ID NO: 75 and a variable light chain (VL) region having an amino acid sequence with at least 80% identity to SEQ ID NO: 79.

88. The monospecific antibody of any one of claims 1-11 and 87, wherein the antibody comprises a variable heavy chain (VH) region having an amino acid sequence with at least 85% identity to SEQ ID NO: 75 and a variable light chain (VL) region having an amino acid sequence with at least 85% identity to SEQ ID NO: 79.

89. The monospecific antibody of any one of claims 1-11, 87, and 88, wherein the antibody comprises a variable heavy chain (VH) region having an amino acid sequence with at least 90% identity to SEQ ID NO: 75 and a variable light chain (VL) region having an amino acid sequence with at least 90% identity to SEQ ID NO: 79.

90. The monospecific antibody of any one of claims 1-11 and 87-89, wherein the antibody comprises a variable heavy chain (VH) region having an amino acid sequence with at least 95% identity to SEQ ID NO: 75 and a variable light chain (VL) region having an amino acid sequence with at least 95% identity to SEQ ID NO: 79.

91. The monospecific antibody of any one of claims 1-11 and 87-90, wherein the antibody comprises a variable heavy chain (VH) region having an amino acid sequence 100% identical to the amino acid sequence of SEQ ID NO: 75 and a variable light chain (VL) region having an amino acid sequence 100% identical to the amino acid sequence of SEQ ID NO: 79.

92. The monospecific antibody of any one of claims 1-11 and 87-91, wherein the antibody comprises a VH comprising therein a complementarity determining region (CDR) H1 having an amino acid sequence 100% identical to the amino acid sequence of SEQ ID NO: 76, a CDR H2 having an amino acid sequence 100% identical to the amino acid sequence of SEQ ID NO: 77, and a CDR H3 having an amino acid sequence 100% identical to the amino acid sequence of SEQ ID NO: 78; and a VL comprising therein a complementarity determining region (CDR) L1 having an amino acid sequence 100% identical to the amino acid sequence of SEQ ID NO: 80, a CDR L2 having an amino acid sequence 100% identical to the amino acid sequence of HAS, and a CDR L3 having an amino acid sequence 100% identical to the amino acid sequence of SEQ ID NO: 81.

93. The monospecific antibody of any one of claims 1-11 and 87-92, wherein the antibody comprises: (a) a variable heavy chain (VH) region having an amino acid sequence with at least 80% identity to SEQ ID NO: 75 and comprising therein a complementarity determining region (CDR) H1 having an amino acid sequence 100% identical to the amino acid sequence of SEQ ID NO: 76, a CDR H2 having an amino acid sequence 100% identical to the amino acid sequence of SEQ ID NO: 77, and a CDR H3 having an amino acid sequence 100% identical to the amino acid sequence of SEQ ID NO: 78; a variable light chain (VL) region having an amino acid sequence with at least 80% identity to SEQ ID NO: 79 and comprising therein a complementarity determining region (CDR) L1 having an amino acid sequence 100% identical to the amino acid sequence of SEQ ID NO: 80, a CDR L2 having an amino acid sequence 100% identical to the amino acid sequence of HAS, and a CDR L3 having an amino acid sequence 100% identical to the amino acid sequence of SEQ ID NO: 81.

94. The monospecific antibody of any one of claims 1-11, wherein the antibody comprises a variable heavy chain (VH) region having an amino acid sequence with at least 80% identity to SEQ ID NO: 82 and a variable light chain (VL) region having an amino acid sequence with at least 80% identity to SEQ ID NO: 86.

95. The monospecific antibody of any one of claims 1-11 and 94, wherein the antibody comprises a variable heavy chain (VH) region having an amino acid sequence with at least 85% identity to SEQ ID NO: 82 and a variable light chain (VL) region having an amino acid sequence with at least 85% identity to SEQ ID NO: 86.

96. The monospecific antibody of any one of claims 1-11, 94, and 95, wherein the antibody comprises a variable heavy chain (VH) region having an amino acid sequence with at least 90% identity to SEQ ID NO: 82 and a variable light chain (VL) region having an amino acid sequence with at least 90% identity to SEQ ID NO: 86.

97. The monospecific antibody of any one of claims 1-11 and 94-96, wherein the antibody comprises a variable heavy chain (VH) region having an amino acid sequence with at least 95% identity to SEQ ID NO: 82 and a variable light chain (VL) region having an amino acid sequence with at least 95% identity to SEQ ID NO: 86.

98. The monospecific antibody of any one of claims 1-11 and 94-97, wherein the antibody comprises a variable heavy chain (VH) region having an amino acid sequence 100% identical to the amino acid sequence of SEQ ID NO: 82 and a variable light chain (VL) region having an amino acid sequence 100% identical to the amino acid sequence of SEQ ID NO: 86.

99. The monospecific antibody of any one of claims 1-11 and 94-98, wherein the antibody comprises a VH comprising therein a complementarity determining region (CDR) H1 having an amino acid sequence 100% identical to the amino acid sequence of SEQ ID NO: 83, a CDR H2 having an amino acid sequence 100% identical to the amino acid sequence of SEQ ID NO: 84, and a CDR H3 having an amino acid sequence 100% identical to the amino acid sequence of SEQ ID NO: 85; and a VL comprising therein a complementarity determining region (CDR) L1 having an amino acid sequence 100% identical to the amino acid sequence of SEQ ID NO: 87, a CDR L2 having an amino acid sequence 100% identical to the amino acid sequence of DAT, and a CDR L3 having an amino acid sequence 100% identical to the amino acid sequence of SEQ ID NO: 88.

100. The monospecific antibody of any one of claims 1-11 and 94-99, wherein the antibody comprises: (a) a variable heavy chain (VH) region having an amino acid sequence with at least 80% identity to SEQ ID NO: 82 and comprising therein a complementarity determining region (CDR) H1 having an amino acid sequence 100% identical to the amino acid sequence of SEQ ID NO: 83, a CDR H2 having an amino acid sequence 100% identical to the amino acid sequence of SEQ ID NO: 84, and a CDR H3 having an amino acid sequence 100% identical to the amino acid sequence of SEQ ID NO: 85; a variable light chain (VL) region having an amino acid sequence with at least 80% identity to SEQ ID NO: 86 and comprising therein a complementarity determining region (CDR) L1 having an amino acid sequence 100% identical to the amino acid sequence of SEQ ID NO: 87, a CDR L2 having an amino acid sequence 100% identical to the amino acid sequence of DAT, and a CDR L3 having an amino acid sequence 100% identical to the amino acid sequence of SEQ ID NO: 88.

101. The monospecific antibody of any one of claims 1-11, wherein the antibody comprises a variable heavy chain (VH) region having an amino acid sequence with at least 80% identity to SEQ ID NO: 89 and a variable light chain (VL) region having an amino acid sequence with at least 80% identity to SEQ ID NO: 93.

102. The monospecific antibody of any one of claims 1-11 and 101, wherein the antibody comprises a variable heavy chain (VH) region having an amino acid sequence with at least 85% identity to SEQ ID NO: 89 and a variable light chain (VL) region having an amino acid sequence with at least 85% identity to SEQ ID NO: 93.

103. The monospecific antibody of any one of claims 1-11, 101, and 102, wherein the antibody comprises a variable heavy chain (VH) region having an amino acid sequence with at least 90% identity to SEQ ID NO: 89 and a variable light chain (VL) region having an amino acid sequence with at least 90% identity to SEQ ID NO: 93.

104. The monospecific antibody of any one of claims 1-11 and 101-103, wherein the antibody comprises a variable heavy chain (VH) region having an amino acid sequence with at least 95% identity to SEQ ID NO: 89 and a variable light chain (VL) region having an amino acid sequence with at least 95% identity to SEQ ID NO: 93.

105. The monospecific antibody of any one of claims 1-11 and 101-104, wherein the antibody comprises a variable heavy chain (VH) region having an amino acid sequence 100% identical to the amino acid sequence of SEQ ID NO: 89 and a variable light chain (VL) region having an amino acid sequence 100% identical to the amino acid sequence of SEQ ID NO: 93.

106. The monospecific antibody of any one of claims 1-11 and 101-105, wherein the antibody comprises a VH comprising therein a complementarity determining region (CDR) H1 having an amino acid sequence 100% identical to the amino acid sequence of SEQ ID NO: 90, a CDR H2 having an amino acid sequence 100% identical to the amino acid sequence of SEQ ID NO: 91, and a CDR H3 having an amino acid sequence 100% identical to the amino acid sequence of SEQ ID NO: 92; and a VL comprising therein a complementarity determining region (CDR) L1 having an amino acid sequence 100% identical to the amino acid sequence of SEQ ID NO: 94, a CDR L2 having an amino acid sequence 100% identical to the amino acid sequence of GAS, and a CDR L3 having an amino acid sequence 100% identical to the amino acid sequence of SEQ ID NO: 95.

107. The monospecific antibody of any one of claims 1-11 and 101-106, wherein the antibody comprises: (a) a variable heavy chain (VH) region having an amino acid sequence with at least 80% identity to SEQ ID NO: 89 and comprising therein a complementarity determining region (CDR) H1 having an amino acid sequence 100% identical to the amino acid sequence of SEQ ID NO: 90, a CDR H2 having an amino acid sequence 100% identical to the amino acid sequence of SEQ ID NO: 91, and a CDR H3 having an amino acid sequence 100% identical to the amino acid sequence of SEQ ID NO: 92; a variable light chain (VL) region having an amino acid sequence with at least 80% identity to SEQ ID NO: 93 and comprising therein a complementarity determining region (CDR) L1 having an amino acid sequence 100% identical to the amino acid sequence of SEQ ID NO: 94, a CDR L2 having an amino acid sequence 100% identical to the amino acid sequence of GAS, and a CDR L3 having an amino acid sequence 100% identical to the amino acid sequence of SEQ ID NO: 95.

108. The monospecific antibody of any one of claims 1-11 and 101-107, wherein the antibody comprises a first heavy chain (HC) region comprising an amino acid sequence having at least 80% identity to SEQ ID NO: 458, a second heavy chain region comprising an amino acid sequence having at least 80% identity to SEQ ID NO: 478, and a light chain (LC) region, comprising an amino acid sequence having at least 80% identity to SEQ ID NO: 468.

109. The monospecific antibody of any one of claims 1-11, wherein the antibody comprises a variable heavy chain (VH) region having an amino acid sequence with at least 80% identity to SEQ ID NO: 96 and a variable light chain (VL) region having an amino acid sequence with at least 80% identity to SEQ ID NO: 100.

110. The monospecific antibody of any one of claims 1-11 and 109, wherein the antibody comprises a variable heavy chain (VH) region having an amino acid sequence with at least 85% identity to SEQ ID NO: 96 and a variable light chain (VL) region having an amino acid sequence with at least 85% identity to SEQ ID NO: 100.

111. The monospecific antibody of any one of claims 1-11, 109, and 110, wherein the antibody comprises a variable heavy chain (VH) region having an amino acid sequence with at least 90% identity to SEQ ID NO: 96 and a variable light chain (VL) region having an amino acid sequence with at least 90% identity to SEQ ID NO: 100.

112. The monospecific antibody of any one of claims 1-11 and 109-111, wherein the antibody comprises a variable heavy chain (VH) region having an amino acid sequence with at least 95% identity to SEQ ID NO: 96 and a variable light chain (VL) region having an amino acid sequence with at least 95% identity to SEQ ID NO: 100.

113. The monospecific antibody of any one of claims 1-11 and 109-112, wherein the antibody comprises a variable heavy chain (VH) region having an amino acid sequence 100% identical to the amino acid sequence of SEQ ID NO: 96 and a variable light chain (VL) region having an amino acid sequence 100% identical to the amino acid sequence of SEQ ID NO: 100.

114. The monospecific antibody of any one of claims 1-11 and 109-113, wherein the antibody comprises a VH comprising therein a complementarity determining region (CDR) H1 having an amino acid sequence 100% identical to the amino acid sequence of SEQ ID NO: 97, a CDR H2 having an amino acid sequence 100% identical to the amino acid sequence of SEQ ID NO: 98, and a CDR H3 having an amino acid sequence 100% identical to the amino acid sequence of SEQ ID NO: 99; and a VL comprising therein a complementarity determining region (CDR) L1 having an amino acid sequence 100% identical to the amino acid sequence of SEQ ID NO: 101, a CDR L2 having an amino acid sequence 100% identical to the amino acid sequence of DAS, and a CDR L3 having an amino acid sequence 100% identical to the amino acid sequence of SEQ ID NO: 102.

115. The monospecific antibody of any one of claims 1-11 and 109-114, wherein the antibody comprises: (a) a variable heavy chain (VH) region having an amino acid sequence with at least 80% identity to SEQ ID NO: 96 and comprising therein a complementarity determining region (CDR) H1 having an amino acid sequence 100% identical to the amino acid sequence of SEQ ID NO: 97, a CDR H2 having an amino acid sequence 100% identical to the amino acid sequence of SEQ ID NO: 98, and a CDR H3 having an amino acid sequence 100% identical to the amino acid sequence of SEQ ID NO: 99; a variable light chain (VL) region having an amino acid sequence with at least 80% identity to SEQ ID NO: 100 and comprising therein a complementarity determining region (CDR) L1 having an amino acid sequence 100% identical to the amino acid sequence of SEQ ID NO: 101, a CDR L2 having an amino acid sequence 100% identical to the amino acid sequence of DAS, and a CDR L3 having an amino acid sequence 100% identical to the amino acid sequence of SEQ ID NO: 102.

116. The monospecific antibody of any one of claims 1-11, wherein the antibody comprises a variable heavy chain (VH) region having an amino acid sequence with at least 80% identity to SEQ ID NO: 103 and a variable light chain (VL) region having an amino acid sequence with at least 80% identity to SEQ ID NO: 107.

117. The monospecific antibody of any one of claims 1-11 and 116, wherein the antibody comprises a variable heavy chain (VH) region having an amino acid sequence with at least 85% identity to SEQ ID NO: 103 and a variable light chain (VL) region having an amino acid sequence with at least 85% identity to SEQ ID NO: 107.

118. The monospecific antibody of any one of claims 1-11, 116, and 117, wherein the antibody comprises a variable heavy chain (VH) region having an amino acid sequence with at least 90% identity to SEQ ID NO: 103 and a variable light chain (VL) region having an amino acid sequence with at least 90% identity to SEQ ID NO: 107.

119. The monospecific antibody of any one of claims 1-11 and 116-118, wherein the antibody comprises a variable heavy chain (VH) region having an amino acid sequence with at least 95% identity to SEQ ID NO: 103 and a variable light chain (VL) region having an amino acid sequence with at least 95% identity to SEQ ID NO: 107.

120. The monospecific antibody of any one of claims 1-11 and 116-119, wherein the antibody comprises a variable heavy chain (VH) region having an amino acid sequence 100% identical to the amino acid sequence of SEQ ID NO: 103 and a variable light chain (VL) region having an amino acid sequence 100% identical to the amino acid sequence of SEQ ID NO: 107.

121. The monospecific antibody of any one of claims 1-11 and 116-120, wherein the antibody comprises a VH comprising therein a complementarity determining region (CDR) H1 having an amino acid sequence 100% identical to the amino acid sequence of SEQ ID NO: 104, a CDR H2 having an amino acid sequence 100% identical to the amino acid sequence of SEQ ID NO: 105, and a CDR H3 having an amino acid sequence 100% identical to the amino acid sequence of SEQ ID NO: 106; and a VL comprising therein a complementarity determining region (CDR) L1 having an amino acid sequence 100% identical to the amino acid sequence of SEQ ID NO: 108, a CDR L2 having an amino acid sequence 100% identical to the amino acid sequence of DIS, and a CDR L3 having an amino acid sequence 100% identical to the amino acid sequence of SEQ ID NO: 109.

122. The monospecific antibody of any one of claims 1-11 and 116-121, wherein the antibody comprises: (a) a variable heavy chain (VH) region having an amino acid sequence with at least 80% identity to SEQ ID NO: 103 and comprising therein a complementarity determining region (CDR) H1 having an amino acid sequence 100% identical to the amino acid sequence of SEQ ID NO: 104, a CDR H2 having an amino acid sequence 100% identical to the amino acid sequence of SEQ ID NO: 105, and a CDR H3 having an amino acid sequence 100% identical to the amino acid sequence of SEQ ID NO: 106; a variable light chain (VL) region having an amino acid sequence with at least 80% identity to SEQ ID NO: 107 and comprising therein a complementarity determining region (CDR) L1 having an amino acid sequence 100% identical to the amino acid sequence of SEQ ID NO: 108, a CDR L2 having an amino acid sequence 100% identical to the amino acid sequence of DIS, and a CDR L3 having an amino acid sequence 100% identical to the amino acid sequence of SEQ ID NO: 109.

123. The monospecific antibody of any one of claims 1-11, wherein the antibody comprises a variable heavy chain (VH) region having an amino acid sequence with at least 80% identity to SEQ ID NO: 110 and a variable light chain (VL) region having an amino acid sequence with at least 80% identity to SEQ ID NO: 114.

124. The monospecific antibody of any one of claims 1-11 and 123, wherein the antibody comprises a variable heavy chain (VH) region having an amino acid sequence with at least 85% identity to SEQ ID NO: 110 and a variable light chain (VL) region having an amino acid sequence with at least 85% identity to SEQ ID NO: 114.

125. The monospecific antibody of any one of claims 1-11, 123, and 124, wherein the antibody comprises a variable heavy chain (VH) region having an amino acid sequence with at least 90% identity to SEQ ID NO: 110 and a variable light chain (VL) region having an amino acid sequence with at least 90% identity to SEQ ID NO: 114.

126. The monospecific antibody of any one of claims 1-11 and 123-125, wherein the antibody comprises a variable heavy chain (VH) region having an amino acid sequence with at least 95% identity to SEQ ID NO: 110 and a variable light chain (VL) region having an amino acid sequence with at least 95% identity to SEQ ID NO: 114.

127. The monospecific antibody of any one of claims 1-11 and 123-126, wherein the antibody comprises a variable heavy chain (VH) region having an amino acid sequence 100% identical to the amino acid sequence of SEQ ID NO: 110 and a variable light chain (VL) region having an amino acid sequence 100% identical to the amino acid sequence of SEQ ID NO: 114.

128. The monospecific antibody of any one of claims 1-11 and 123-127, wherein the antibody comprises a VH comprising therein a complementarity determining region (CDR) H1 having an amino acid sequence 100% identical to the amino acid sequence of SEQ ID NO: 111, a CDR H2 having an amino acid sequence 100% identical to the amino acid sequence of SEQ ID NO: 112, and a CDR H3 having an amino acid sequence 100% identical to the amino acid sequence of SEQ ID NO: 113; and a VL comprising therein a complementarity determining region (CDR) L1 having an amino acid sequence 100% identical to the amino acid sequence of SEQ ID NO: 115, a CDR L2 having an amino acid sequence 100% identical to the amino acid sequence of DAS, and a CDR L3 having an amino acid sequence 100% identical to the amino acid sequence of SEQ ID NO: 116.

129. The monospecific antibody of any one of claims 1-11 and 123-128, wherein the antibody comprises: (a) a variable heavy chain (VH) region having an amino acid sequence with at least 80% identity to SEQ ID NO: 110 and comprising therein a complementarity determining region (CDR) H1 having an amino acid sequence 100% identical to the amino acid sequence of SEQ ID NO: 111, a CDR H2 having an amino acid sequence 100% identical to the amino acid sequence of SEQ ID NO: 112, and a CDR H3 having an amino acid sequence 100% identical to the amino acid sequence of SEQ ID NO: 113; a variable light chain (VL) region having an amino acid sequence with at least 80% identity to SEQ ID NO: 114 and comprising therein a complementarity determining region (CDR) L1 having an amino acid sequence 100% identical to the amino acid sequence of SEQ ID NO: 115, a CDR L2 having an amino acid sequence 100% identical to the amino acid sequence of DAS, and a CDR L3 having an amino acid sequence 100% identical to the amino acid sequence of SEQ ID NO: 116.

130. The monospecific antibody of any one of claims 1-11 and 123-129, wherein the antibody comprises a first heavy chain (HC) region comprising an amino acid sequence having at least 80% identity to SEQ ID NO: 459, a second heavy chain region comprising an amino acid sequence having at least 80% identity to SEQ ID NO: 479, and a light chain (LC) region, comprising an amino acid sequence having at least 80% identity to SEQ ID NO: 469.

131. The monospecific antibody of any one of claims 1-11, wherein the antibody comprises a variable heavy chain (VH) region having an amino acid sequence with at least 80% identity to SEQ ID NO: 117 and a variable light chain (VL) region having an amino acid sequence with at least 80% identity to SEQ ID NO: 121.

132. The monospecific antibody of any one of claims 1-11 and 131, wherein the antibody comprises a variable heavy chain (VH) region having an amino acid sequence with at least 85% identity to SEQ ID NO: 117 and a variable light chain (VL) region having an amino acid sequence with at least 85% identity to SEQ ID NO: 121.

133. The monospecific antibody of any one of claims 1-11, 131, and 132, wherein the antibody comprises a variable heavy chain (VH) region having an amino acid sequence with at least 90% identity to SEQ ID NO: 117 and a variable light chain (VL) region having an amino acid sequence with at least 90% identity to SEQ ID NO: 121.

134. The monospecific antibody of any one of claims 1-11 and 131-133, wherein the antibody comprises a variable heavy chain (VH) region having an amino acid sequence with at least 95% identity to SEQ ID NO: 117 and a variable light chain (VL) region having an amino acid sequence with at least 95% identity to SEQ ID NO: 121.

135. The monospecific antibody of any one of claims 1-11 and 131-134, wherein the antibody comprises a variable heavy chain (VH) region having an amino acid sequence 100% identical to the amino acid sequence of SEQ ID NO: 117 and a variable light chain (VL) region having an amino acid sequence 100% identical to the amino acid sequence of SEQ ID NO: 121.

136. The monospecific antibody of any one of claims 1-11 and 131-135, wherein the antibody comprises a VH comprising therein a complementarity determining region (CDR) H1 having an amino acid sequence 100% identical to the amino acid sequence of SEQ ID NO: 118, a CDR H2 having an amino acid sequence 100% identical to the amino acid sequence of SEQ ID NO: 119, and a CDR H3 having an amino acid sequence 100% identical to the amino acid sequence of SEQ ID NO: 120; and a VL comprising therein a complementarity determining region (CDR) L1 having an amino acid sequence 100% identical to the amino acid sequence of SEQ ID NO: 122, a CDR L2 having an amino acid sequence 100% identical to the amino acid sequence of DAS, and a CDR L3 having an amino acid sequence 100% identical to the amino acid sequence of SEQ ID NO: 123.

137. The monospecific antibody of any one of claims 1-11 and 131-136, wherein the antibody comprises: (a) a variable heavy chain (VH) region having an amino acid sequence with at least 80% identity to SEQ ID NO: 117 and comprising therein a complementarity determining region (CDR) H1 having an amino acid sequence 100% identical to the amino acid sequence of SEQ ID NO: 118, a CDR H2 having an amino acid sequence 100% identical to the amino acid sequence of SEQ ID NO: 119, and a CDR H3 having an amino acid sequence 100% identical to the amino acid sequence of SEQ ID NO: 120; a variable light chain (VL) region having an amino acid sequence with at least 80% identity to SEQ ID NO: 121 and comprising therein a complementarity determining region (CDR) L1 having an amino acid sequence 100% identical to the amino acid sequence of SEQ ID NO: 122, a CDR L2 having an amino acid sequence 100% identical to the amino acid sequence of DAS, and a CDR L3 having an amino acid sequence 100% identical to the amino acid sequence of SEQ ID NO: 123.

138. The monospecific antibody of any one of claims 1-11, wherein the antibody comprises a variable heavy chain (VH) region having an amino acid sequence with at least 80% identity to SEQ ID NO: 124 and a variable light chain (VL) region having an amino acid sequence with at least 80% identity to SEQ ID NO: 128.

139. The monospecific antibody of any one of claims 1-11 and 138, wherein the antibody comprises a variable heavy chain (VH) region having an amino acid sequence with at least 85% identity to SEQ ID NO: 124 and a variable light chain (VL) region having an amino acid sequence with at least 85% identity to SEQ ID NO: 128.

140. The monospecific antibody of any one of claims 1-11, 138, and 139, wherein the antibody comprises a variable heavy chain (VH) region having an amino acid sequence with at least 90% identity to SEQ ID NO: 124 and a variable light chain (VL) region having an amino acid sequence with at least 90% identity to SEQ ID NO: 128.

141. The monospecific antibody of any one of claims 1-11 and 138-140, wherein the antibody comprises a variable heavy chain (VH) region having an amino acid sequence with at least 95% identity to SEQ ID NO: 124 and a variable light chain (VL) region having an amino acid sequence with at least 95% identity to SEQ ID NO: 128.

142. The monospecific antibody of any one of claims 1-11 and 138-141, wherein the antibody comprises a variable heavy chain (VH) region having an amino acid sequence 100% identical to the amino acid sequence of SEQ ID NO: 124 and a variable light chain (VL) region having an amino acid sequence 100% identical to the amino acid sequence of SEQ ID NO: 128.

143. The monospecific antibody of any one of claims 1-11 and 138-142, wherein the antibody comprises a VH comprising therein a complementarity determining region (CDR) H1 having an amino acid sequence 100% identical to the amino acid sequence of SEQ ID NO: 125, a CDR H2 having an amino acid sequence 100% identical to the amino acid sequence of SEQ ID NO: 126, and a CDR H3 having an amino acid sequence 100% identical to the amino acid sequence of SEQ ID NO: 127; and a VL comprising therein a complementarity determining region (CDR) L1 having an amino acid sequence 100% identical to the amino acid sequence of SEQ ID NO: 129, a CDR L2 having an amino acid sequence 100% identical to the amino acid sequence of EAS, and a CDR L3 having an amino acid sequence 100% identical to the amino acid sequence of SEQ ID NO: 130.

144. The monospecific antibody of any one of claims 1-11 and 138-143, wherein the antibody comprises: (a) a variable heavy chain (VH) region having an amino acid sequence with at least 80% identity to SEQ ID NO: 124 and comprising therein a complementarity determining region (CDR) H1 having an amino acid sequence 100% identical to the amino acid sequence of SEQ ID NO: 125, a CDR H2 having an amino acid sequence 100% identical to the amino acid sequence of SEQ ID NO: 126, and a CDR H3 having an amino acid sequence 100% identical to the amino acid sequence of SEQ ID NO: 127; a variable light chain (VL) region having an amino acid sequence with at least 80% identity to SEQ ID NO: 128 and comprising therein a complementarity determining region (CDR) L1 having an amino acid sequence 100% identical to the amino acid sequence of SEQ ID NO: 129, a CDR L2 having an amino acid sequence 100% identical to the amino acid sequence of EAS, and a CDR L3 having an amino acid sequence 100% identical to the amino acid sequence of SEQ ID NO: 131.

145. The monospecific antibody of any one of claims 1-11, wherein the antibody comprises a variable heavy chain (VH) region having an amino acid sequence with at least 80% identity to SEQ ID NO: 131 and a variable light chain (VL) region having an amino acid sequence with at least 80% identity to SEQ ID NO: 135.

146. The monospecific antibody of any one of claims 1-11 and 145, wherein the antibody comprises a variable heavy chain (VH) region having an amino acid sequence with at least 85% identity to SEQ ID NO: 131 and a variable light chain (VL) region having an amino acid sequence with at least 85% identity to SEQ ID NO: 135.

147. The monospecific antibody of any one of claims 1-11, 145, and 146, wherein the antibody comprises a variable heavy chain (VH) region having an amino acid sequence with at least 90% identity to SEQ ID NO: 131 and a variable light chain (VL) region having an amino acid sequence with at least 90% identity to SEQ ID NO: 135.

148. The monospecific antibody of any one of claims 1-11 and 145-147, wherein the antibody comprises a variable heavy chain (VH) region having an amino acid sequence with at least 95% identity to SEQ ID NO: 131 and a variable light chain (VL) region having an amino acid sequence with at least 95% identity to SEQ ID NO: 135.

149. The monospecific antibody of any one of claims 1-11 and 145-148, wherein the antibody comprises a variable heavy chain (VH) region having an amino acid sequence 100% identical to the amino acid sequence of SEQ ID NO: 131 and a variable light chain (VL) region having an amino acid sequence 100% identical to the amino acid sequence of SEQ ID NO: 135.

150. The monospecific antibody of any one of claims 1-11 and 145-149, wherein the antibody comprises a VH comprising therein a complementarity determining region (CDR) H1 having an amino acid sequence 100% identical to the amino acid sequence of SEQ ID NO: 132, a CDR H2 having an amino acid sequence 100% identical to the amino acid sequence of SEQ ID NO: 133, and a CDR H3 having an amino acid sequence 100% identical to the amino acid sequence of SEQ ID NO: 134; and a VL comprising therein a complementarity determining region (CDR) L1 having an amino acid sequence 100% identical to the amino acid sequence of SEQ ID NO: 136, a CDR L2 having an amino acid sequence 100% identical to the amino acid sequence of DAS, and a CDR L3 having an amino acid sequence 100% identical to the amino acid sequence of SEQ ID NO: 137.

151. The monospecific antibody of any one of claims 1-11 and 145-150, wherein the antibody comprises: (a) a variable heavy chain (VH) region having an amino acid sequence with at least 80% identity to SEQ ID NO: 131 and comprising therein a complementarity determining region (CDR) H1 having an amino acid sequence 100% identical to the amino acid sequence of SEQ ID NO: 132, a CDR H2 having an amino acid sequence 100% identical to the amino acid sequence of SEQ ID NO: 133, and a CDR H3 having an amino acid sequence 100% identical to the amino acid sequence of SEQ ID NO: 134; a variable light chain (VL) region having an amino acid sequence with at least 80% identity to SEQ ID NO: 135 and comprising therein a complementarity determining region (CDR) L1 having an amino acid sequence 100% identical to the amino acid sequence of SEQ ID NO: 136, a CDR L2 having an amino acid sequence 100% identical to the amino acid sequence of DAS, and a CDR L3 having an amino acid sequence 100% identical to the amino acid sequence of SEQ ID NO: 137.

152. The monospecific antibody of any one of claims 1-11, wherein the antibody comprises a variable heavy chain (VH) region having an amino acid sequence with at least 80% identity to SEQ ID NO: 138 and a variable light chain (VL) region having an amino acid sequence with at least 80% identity to SEQ ID NO: 142.

153. The monospecific antibody of any one of claims 1-11 and 152, wherein the antibody comprises a variable heavy chain (VH) region having an amino acid sequence with at least 85% identity to SEQ ID NO: 138 and a variable light chain (VL) region having an amino acid sequence with at least 85% identity to SEQ ID NO: 142.

154. The monospecific antibody of any one of claims 1-11, 152, and 153, wherein the antibody comprises a variable heavy chain (VH) region having an amino acid sequence with at least 90% identity to SEQ ID NO: 138 and a variable light chain (VL) region having an amino acid sequence with at least 90% identity to SEQ ID NO: 142.

155. The monospecific antibody of any one of claims 1-11 and 152-153, wherein the antibody comprises a variable heavy chain (VH) region having an amino acid sequence with at least 95% identity to SEQ ID NO: 138 and a variable light chain (VL) region having an amino acid sequence with at least 95% identity to SEQ ID NO: 142.

156. The monospecific antibody of any one of claims 1-11 and 152-154, wherein the antibody comprises a variable heavy chain (VH) region having an amino acid sequence 100% identical to the amino acid sequence of SEQ ID NO: 138 and a variable light chain (VL) region having an amino acid sequence 100% identical to the amino acid sequence of SEQ ID NO: 142.

157. The monospecific antibody of any one of claims 1-11 and 152-156, wherein the antibody comprises a VH comprising therein a complementarity determining region (CDR) H1 having an amino acid sequence 100% identical to the amino acid sequence of SEQ ID NO: 139, a CDR H2 having an amino acid sequence 100% identical to the amino acid sequence of SEQ ID NO: 140, and a CDR H3 having an amino acid sequence 100% identical to the amino acid sequence of SEQ ID NO: 141; and a VL comprising therein a complementarity determining region (CDR) L1 having an amino acid sequence 100% identical to the amino acid sequence of SEQ ID NO: 143, a CDR L2 having an amino acid sequence 100% identical to the amino acid sequence of DAS, and a CDR L3 having an amino acid sequence 100% identical to the amino acid sequence of SEQ ID NO: 144.

158. The monospecific antibody of any one of claims 1-11 and 152-157, wherein the antibody comprises: (a) a variable heavy chain (VH) region having an amino acid sequence with at least 80% identity to SEQ ID NO: 138 and comprising therein a complementarity determining region (CDR) H1 having an amino acid sequence 100% identical to the amino acid sequence of SEQ ID NO: 139, a CDR H2 having an amino acid sequence 100% identical to the amino acid sequence of SEQ ID NO: 140, and a CDR H3 having an amino acid sequence 100% identical to the amino acid sequence of SEQ ID NO: 141; a variable light chain (VL) region having an amino acid sequence with at least 80% identity to SEQ ID NO: 142 and comprising therein a complementarity determining region (CDR) L1 having an amino acid sequence 100% identical to the amino acid sequence of SEQ ID NO: 143, a CDR L2 having an amino acid sequence 100% identical to the amino acid sequence of DAS, and a CDR L3 having an amino acid sequence 100% identical to the amino acid sequence of SEQ ID NO: 144.

159. The monospecific antibody of any one of claims 1-11, wherein the antibody comprises a variable heavy chain (VH) region having an amino acid sequence with at least 80% identity to SEQ ID NO: 145 and a variable light chain (VL) region having an amino acid sequence with at least 80% identity to SEQ ID NO: 149.

160. The monospecific antibody of any one of claims 1-11 and 159, wherein the antibody comprises a variable heavy chain (VH) region having an amino acid sequence with at least 85% identity to SEQ ID NO: 145 and a variable light chain (VL) region having an amino acid sequence with at least 85% identity to SEQ ID NO: 149.

161. The monospecific antibody of any one of claims 1-11, 159, and 160, wherein the antibody comprises a variable heavy chain (VH) region having an amino acid sequence with at least 90% identity to SEQ ID NO: 145 and a variable light chain (VL) region having an amino acid sequence with at least 90% identity to SEQ ID NO: 149.

162. The monospecific antibody of any one of claims 1-11 and 159-161, wherein the antibody comprises a variable heavy chain (VH) region having an amino acid sequence with at least 95% identity to SEQ ID NO: 145 and a variable light chain (VL) region having an amino acid sequence with at least 95% identity to SEQ ID NO: 149.

163. The monospecific antibody of any one of claims 1-11 and 159-162, wherein the antibody comprises a variable heavy chain (VH) region having an amino acid sequence 100% identical to the amino acid sequence of SEQ ID NO: 145 and a variable light chain (VL) region having an amino acid sequence 100% identical to the amino acid sequence of SEQ ID NO: 149.

164. The monospecific antibody of any one of claims 1-11 and 159-163, wherein the antibody comprises a VH comprising therein a complementarity determining region (CDR) H1 having an amino acid sequence 100% identical to the amino acid sequence of SEQ ID NO: 146, a CDR H2 having an amino acid sequence 100% identical to the amino acid sequence of SEQ ID NO: 147, and a CDR H3 having an amino acid sequence 100% identical to the amino acid sequence of SEQ ID NO: 148; and a VL comprising therein a complementarity determining region (CDR) L1 having an amino acid sequence 100% identical to the amino acid sequence of SEQ ID NO: 150, a CDR L2 having an amino acid sequence 100% identical to the amino acid sequence of DAS, and a CDR L3 having an amino acid sequence 100% identical to the amino acid sequence of SEQ ID NO: 151.

165. The monospecific antibody of any one of claims 1-11 and 159-164, wherein the antibody comprises: (a) a variable heavy chain (VH) region having an amino acid sequence with at least 80% identity to SEQ ID NO: 145 and comprising therein a complementarity determining region (CDR) H1 having an amino acid sequence 100% identical to the amino acid sequence of SEQ ID NO: 146, a CDR H2 having an amino acid sequence 100% identical to the amino acid sequence of SEQ ID NO: 147, and a CDR H3 having an amino acid sequence 100% identical to the amino acid sequence of SEQ ID NO: 148; a variable light chain (VL) region having an amino acid sequence with at least 80% identity to SEQ ID NO: 149 and comprising therein a complementarity determining region (CDR) L1 having an amino acid sequence 100% identical to the amino acid sequence of SEQ ID NO: 150, a CDR L2 having an amino acid sequence 100% identical to the amino acid sequence of DAS, and a CDR L3 having an amino acid sequence 100% identical to the amino acid sequence of SEQ ID NO: 151.

166. The monospecific antibody of any one of claims 1-11, wherein the antibody comprises a variable heavy chain (VH) region having an amino acid sequence with at least 80% identity to SEQ ID NO: 152 and a variable light chain (VL) region having an amino acid sequence with at least 80% identity to SEQ ID NO: 156.

167. The monospecific antibody of any one of claims 1-11 and 166, wherein the antibody comprises a variable heavy chain (VH) region having an amino acid sequence with at least 85% identity to SEQ ID NO: 152 and a variable light chain (VL) region having an amino acid sequence with at least 85% identity to SEQ ID NO: 156.

168. The monospecific antibody of any one of claims 1-11, 166, and 167, wherein the antibody comprises a variable heavy chain (VH) region having an amino acid sequence with at least 90% identity to SEQ ID NO: 152 and a variable light chain (VL) region having an amino acid sequence with at least 90% identity to SEQ ID NO: 156.

169. The monospecific antibody of any one of claims 1-11 and 166-168, wherein the antibody comprises a variable heavy chain (VH) region having an amino acid sequence with at least 95% identity to SEQ ID NO: 152 and a variable light chain (VL) region having an amino acid sequence with at least 95% identity to SEQ ID NO: 156.

170. The monospecific antibody of any one of claims 1-11 and 166-169, wherein the antibody comprises a variable heavy chain (VH) region having an amino acid sequence 100% identical to the amino acid sequence of SEQ ID NO: 152 and a variable light chain (VL) region having an amino acid sequence 100% identical to the amino acid sequence of SEQ ID NO: 156.

171. The monospecific antibody of any one of claims 1-11 and 166-170, wherein the antibody comprises a VH comprising therein a complementarity determining region (CDR) H1 having an amino acid sequence 100% identical to the amino acid sequence of SEQ ID NO: 153, a CDR H2 having an amino acid sequence 100% identical to the amino acid sequence of SEQ ID NO: 154, and a CDR H3 having an amino acid sequence 100% identical to the amino acid sequence of SEQ ID NO: 155; and a VL comprising therein a complementarity determining region (CDR) L1 having an amino acid sequence 100% identical to the amino acid sequence of SEQ ID NO: 157, a CDR L2 having an amino acid sequence 100% identical to the amino acid sequence of DAS, and a CDR L3 having an amino acid sequence 100% identical to the amino acid sequence of SEQ ID NO: 158.

172. The monospecific antibody of any one of claims 1-11 and 166-171, wherein the antibody comprises: (a) a variable heavy chain (VH) region having an amino acid sequence with at least 80% identity to SEQ ID NO: 152 and comprising therein a complementarity determining region (CDR) H1 having an amino acid sequence 100% identical to the amino acid sequence of SEQ ID NO: 153, a CDR H2 having an amino acid sequence 100% identical to the amino acid sequence of SEQ ID NO: 154, and a CDR H3 having an amino acid sequence 100% identical to the amino acid sequence of SEQ ID NO: 155; a variable light chain (VL) region having an amino acid sequence with at least 80% identity to SEQ ID NO: 156 and comprising therein a complementarity determining region (CDR) L1 having an amino acid sequence 100% identical to the amino acid sequence of SEQ ID NO: 157, a CDR L2 having an amino acid sequence 100% identical to the amino acid sequence of DAS, and a CDR L3 having an amino acid sequence 100% identical to the amino acid sequence of SEQ ID NO: 158.

173. The monospecific antibody of any one of claims 1-11, wherein the antibody comprises a variable heavy chain (VH) region having an amino acid sequence with at least 80% identity to SEQ ID NO: 159 and a variable light chain (VL) region having an amino acid sequence with at least 80% identity to SEQ ID NO: 163.

174. The monospecific antibody of any one of claims 1-11 and 173, wherein the antibody comprises a variable heavy chain (VH) region having an amino acid sequence with at least 85% identity to SEQ ID NO: 159 and a variable light chain (VL) region having an amino acid sequence with at least 85% identity to SEQ ID NO: 163.

175. The monospecific antibody of any one of claims 1-11, 173, and 173, wherein the antibody comprises a variable heavy chain (VH) region having an amino acid sequence with at least 90% identity to SEQ ID NO: 159 and a variable light chain (VL) region having an amino acid sequence with at least 90% identity to SEQ ID NO: 163.

176. The monospecific antibody of any one of claims 1-11 and 173-175, wherein the antibody comprises a variable heavy chain (VH) region having an amino acid sequence with at least 95% identity to SEQ ID NO: 159 and a variable light chain (VL) region having an amino acid sequence with at least 95% identity to SEQ ID NO: 163.

177. The monospecific antibody of any one of claims 1-11 and 173-176, wherein the antibody comprises a variable heavy chain (VH) region having an amino acid sequence 100% identical to the amino acid sequence of SEQ ID NO: 159 and a variable light chain (VL) region having an amino acid sequence 100% identical to the amino acid sequence of SEQ ID NO: 163.

178. The monospecific antibody of any one of claims 1-11 and 173-177, wherein the antibody comprises a VH comprising therein a complementarity determining region (CDR) H1 having an amino acid sequence 100% identical to the amino acid sequence of SEQ ID NO: 160, a CDR H2 having an amino acid sequence 100% identical to the amino acid sequence of SEQ ID NO: 161, and a CDR H3 having an amino acid sequence 100% identical to the amino acid sequence of SEQ ID NO: 162; and a VL comprising therein a complementarity determining region (CDR) L1 having an amino acid sequence 100% identical to the amino acid sequence of SEQ ID NO: 164, a CDR L2 having an amino acid sequence 100% identical to the amino acid sequence of DAS, and a CDR L3 having an amino acid sequence 100% identical to the amino acid sequence of SEQ ID NO: 165.

179. The monospecific antibody of any one of claims 1-11 and 173-178, wherein the antibody comprises: (a) a variable heavy chain (VH) region having an amino acid sequence with at least 80% identity to SEQ ID NO: 159 and comprising therein a complementarity determining region (CDR) H1 having an amino acid sequence 100% identical to the amino acid sequence of SEQ ID NO: 160, a CDR H2 having an amino acid sequence 100% identical to the amino acid sequence of SEQ ID NO: 161, and a CDR H3 having an amino acid sequence 100% identical to the amino acid sequence of SEQ ID NO: 162; a variable light chain (VL) region having an amino acid sequence with at least 80% identity to SEQ ID NO: 163 and comprising therein a complementarity determining region (CDR) L1 having an amino acid sequence 100% identical to the amino acid sequence of SEQ ID NO: 164, a CDR L2 having an amino acid sequence 100% identical to the amino acid sequence of DAS, and a CDR L3 having an amino acid sequence 100% identical to the amino acid sequence of SEQ ID NO: 165.

180. The monospecific antibody of any one of claims 1-11, wherein the antibody comprises a variable heavy chain (VH) region having an amino acid sequence with at least 80% identity to SEQ ID NO: 166 and a variable light chain (VL) region having an amino acid sequence with at least 80% identity to SEQ ID NO: 170.

181. The monospecific antibody of any one of claims 1-11 and 180, wherein the antibody comprises a variable heavy chain (VH) region having an amino acid sequence with at least 85% identity to SEQ ID NO: 166 and a variable light chain (VL) region having an amino acid sequence with at least 85% identity to SEQ ID NO: 170.

182. The monospecific antibody of any one of claims 1-11, 180, and 181, wherein the antibody comprises a variable heavy chain (VH) region having an amino acid sequence with at least 90% identity to SEQ ID NO: 166 and a variable light chain (VL) region having an amino acid sequence with at least 90% identity to SEQ ID NO: 170.

183. The monospecific antibody of any one of claims 1-11 and 180-182, wherein the antibody comprises a variable heavy chain (VH) region having an amino acid sequence with at least 95% identity to SEQ ID NO: 166 and a variable light chain (VL) region having an amino acid sequence with at least 95% identity to SEQ ID NO: 170.

184. The monospecific antibody of any one of claims 1-11 and 180-183, wherein the antibody comprises a variable heavy chain (VH) region having an amino acid sequence 100% identical to the amino acid sequence of SEQ ID NO: 166 and a variable light chain (VL) region having an amino acid sequence 100% identical to the amino acid sequence of SEQ ID NO: 170.

185. The monospecific antibody of any one of claims 1-11 and 180-184, wherein the antibody comprises a VH comprising therein a complementarity determining region (CDR) H1 having an amino acid sequence 100% identical to the amino acid sequence of SEQ ID NO: 167, a CDR H2 having an amino acid sequence 100% identical to the amino acid sequence of SEQ ID NO: 168, and a CDR H3 having an amino acid sequence 100% identical to the amino acid sequence of SEQ ID NO: 169; and a VL comprising therein a complementarity determining region (CDR) L1 having an amino acid sequence 100% identical to the amino acid sequence of SEQ ID NO: 171, a CDR L2 having an amino acid sequence 100% identical to the amino acid sequence of GAS, and a CDR L3 having an amino acid sequence 100% identical to the amino acid sequence of SEQ ID NO: 172.

186. The monospecific antibody of any one of claims 1-11 and 180-185, wherein the antibody comprises: (a) a variable heavy chain (VH) region having an amino acid sequence with at least 80% identity to SEQ ID NO: 166 and comprising therein a complementarity determining region (CDR) H1 having an amino acid sequence 100% identical to the amino acid sequence of SEQ ID NO: 167, a CDR H2 having an amino acid sequence 100% identical to the amino acid sequence of SEQ ID NO: 168, and a CDR H3 having an amino acid sequence 100% identical to the amino acid sequence of SEQ ID NO: 169; a variable light chain (VL) region having an amino acid sequence with at least 80% identity to SEQ ID NO: 170 and comprising therein a complementarity determining region (CDR) L1 having an amino acid sequence 100% identical to the amino acid sequence of SEQ ID NO: 171, a CDR L2 having an amino acid sequence 100% identical to the amino acid sequence of GAS, and a CDR L3 having an amino acid sequence 100% identical to the amino acid sequence of SEQ ID NO: 172.

187. The monospecific antibody of any one of claims 1-11, wherein the antibody comprises a variable heavy chain (VH) region having an amino acid sequence with at least 80% identity to SEQ ID NO: 173 and a variable light chain (VL) region having an amino acid sequence with at least 80% identity to SEQ ID NO: 177.

188. The monospecific antibody of any one of claims 1-11, and 187, wherein the antibody comprises a variable heavy chain (VH) region having an amino acid sequence with at least 85% identity to SEQ ID NO: 173 and a variable light chain (VL) region having an amino acid sequence with at least 85% identity to SEQ ID NO: 177.

189. The monospecific antibody of any one of claims 1-11, 187 and 187-188, wherein the antibody comprises a variable heavy chain (VH) region having an amino acid sequence with at least 90% identity to SEQ ID NO: 173 and a variable light chain (VL) region having an amino acid sequence with at least 90% identity to SEQ ID NO: 177.

190. The monospecific antibody of any one of claims 1-11 and 187-189, wherein the antibody comprises a variable heavy chain (VH) region having an amino acid sequence with at least 95% identity to SEQ ID NO: 173 and a variable light chain (VL) region having an amino acid sequence with at least 95% identity to SEQ ID NO: 177.

191. The monospecific antibody of any one of claims 1-11 and 187-190, wherein the antibody comprises a variable heavy chain (VH) region having an amino acid sequence 100% identical to the amino acid sequence of SEQ ID NO: 173 and a variable light chain (VL) region having an amino acid sequence 100% identical to the amino acid sequence of SEQ ID NO: 177.

192. The monospecific antibody of any one of claims 1-11 and 187-191, wherein the antibody comprises a VH comprising therein a complementarity determining region (CDR) H1 having an amino acid sequence 100% identical to the amino acid sequence of SEQ ID NO: 174, a CDR H2 having an amino acid sequence 100% identical to the amino acid sequence of SEQ ID NO: 175, and a CDR H3 having an amino acid sequence 100% identical to the amino acid sequence of SEQ ID NO: 176; and a VL comprising therein a complementarity determining region (CDR) L1 having an amino acid sequence 100% identical to the amino acid sequence of SEQ ID NO: 178, a CDR L2 having an amino acid sequence 100% identical to the amino acid sequence of GAS, and a CDR L3 having an amino acid sequence 100% identical to the amino acid sequence of SEQ ID NO: 179.

193. The monospecific antibody of any one of claims 1-11 and 187-192, wherein the antibody comprises: (a) a variable heavy chain (VH) region having an amino acid sequence with at least 80% identity to SEQ ID NO: 173 and comprising therein a complementarity determining region (CDR) H1 having an amino acid sequence 100% identical to the amino acid sequence of SEQ ID NO: 174, a CDR H2 having an amino acid sequence 100% identical to the amino acid sequence of SEQ ID NO: 175, and a CDR H3 having an amino acid sequence 100% identical to the amino acid sequence of SEQ ID NO: 176; a variable light chain (VL) region having an amino acid sequence with at least 80% identity to SEQ ID NO: 177 and comprising therein a complementarity determining region (CDR) L1 having an amino acid sequence 100% identical to the amino acid sequence of SEQ ID NO: 178, a CDR L2 having an amino acid sequence 100% identical to the amino acid sequence of GAS, and a CDR L3 having an amino acid sequence 100% identical to the amino acid sequence of SEQ ID NO: 179.

194. The monospecific antibody of any one of claims 1-11, wherein the antibody comprises a variable heavy chain (VH) region having an amino acid sequence with at least 80% identity to SEQ ID NO: 180 and a variable light chain (VL) region having an amino acid sequence with at least 80% identity to SEQ ID NO: 184.

195. The monospecific antibody of any one of claims 1-11 and 194, wherein the antibody comprises a variable heavy chain (VH) region having an amino acid sequence with at least 85% identity to SEQ ID NO: 180 and a variable light chain (VL) region having an amino acid sequence with at least 85% identity to SEQ ID NO: 184.

196. The monospecific antibody of any one of claims 1-11, 194, and 195, wherein the antibody comprises a variable heavy chain (VH) region having an amino acid sequence with at least 90% identity to SEQ ID NO: 180 and a variable light chain (VL) region having an amino acid sequence with at least 90% identity to SEQ ID NO: 184.

197. The monospecific antibody of any one of claims 1-11 and 194-196, wherein the antibody comprises a variable heavy chain (VH) region having an amino acid sequence with at least 95% identity to SEQ ID NO: 180 and a variable light chain (VL) region having an amino acid sequence with at least 95% identity to SEQ ID NO: 184.

198. The monospecific antibody of any one of claims 1-11 and 194-197, wherein the antibody comprises a variable heavy chain (VH) region having an amino acid sequence 100% identical to the amino acid sequence of SEQ ID NO: 180 and a variable light chain (VL) region having an amino acid sequence 100% identical to the amino acid sequence of SEQ ID NO: 184.

199. The monospecific antibody of any one of claims 1-11 and 194-198, wherein the antibody comprises a VH comprising therein a complementarity determining region (CDR) H1 having an amino acid sequence 100% identical to the amino acid sequence of SEQ ID NO: 181, a CDR H2 having an amino acid sequence 100% identical to the amino acid sequence of SEQ ID NO: 182, and a CDR H3 having an amino acid sequence 100% identical to the amino acid sequence of SEQ ID NO: 183; and a VL comprising therein a complementarity determining region (CDR) L1 having an amino acid sequence 100% identical to the amino acid sequence of SEQ ID NO: 185, a CDR L2 having an amino acid sequence 100% identical to the amino acid sequence of DVS, and a CDR L3 having an amino acid sequence 100% identical to the amino acid sequence of SEQ ID NO: 186.

200. The monospecific antibody of any one of claims 1-11 and 194-199, wherein the antibody comprises: (a) a variable heavy chain (VH) region having an amino acid sequence with at least 80% identity to SEQ ID NO: 180 and comprising therein a complementarity determining region (CDR) H1 having an amino acid sequence 100% identical to the amino acid sequence of SEQ ID NO: 181, a CDR H2 having an amino acid sequence 100% identical to the amino acid sequence of SEQ ID NO: 182, and a CDR H3 having an amino acid sequence 100% identical to the amino acid sequence of SEQ ID NO: 183; a variable light chain (VL) region having an amino acid sequence with at least 80% identity to SEQ ID NO: 184 and comprising therein a complementarity determining region (CDR) L1 having an amino acid sequence 100% identical to the amino acid sequence of SEQ ID NO: 185, a CDR L2 having an amino acid sequence 100% identical to the amino acid sequence of DVS, and a CDR L3 having an amino acid sequence 100% identical to the amino acid sequence of SEQ ID NO: 186.

201. The monospecific antibody of any one of claims 1-11, wherein the antibody comprises a variable heavy chain (VH) region having an amino acid sequence with at least 80% identity to SEQ ID NO: 187 and a variable light chain (VL) region having an amino acid sequence with at least 80% identity to SEQ ID NO: 191.

202. The monospecific antibody of any one of claims 1-11 and 201, wherein the antibody comprises a variable heavy chain (VH) region having an amino acid sequence with at least 85% identity to SEQ ID NO: 187 and a variable light chain (VL) region having an amino acid sequence with at least 85% identity to SEQ ID NO: 191.

203. The monospecific antibody of any one of claims 1-11, 201, and 202, wherein the antibody comprises a variable heavy chain (VH) region having an amino acid sequence with at least 90% identity to SEQ ID NO: 187 and a variable light chain (VL) region having an amino acid sequence with at least 90% identity to SEQ ID NO: 191.

204. The monospecific antibody of any one of claims 1-11 and 201-203, wherein the antibody comprises a variable heavy chain (VH) region having an amino acid sequence with at least 95% identity to SEQ ID NO: 187 and a variable light chain (VL) region having an amino acid sequence with at least 95% identity to SEQ ID NO: 191.

205. The monospecific antibody of any one of claims 1-11 and 201-204, wherein the antibody comprises a variable heavy chain (VH) region having an amino acid sequence 100% identical to the amino acid sequence of SEQ ID NO: 187 and a variable light chain (VL) region having an amino acid sequence 100% identical to the amino acid sequence of SEQ ID NO: 191.

206. The monospecific antibody of any one of claims 1-11 and 201-205, wherein the antibody comprises a VH comprising therein a complementarity determining region (CDR) H1 having an amino acid sequence 100% identical to the amino acid sequence of SEQ ID NO: 188, a CDR H2 having an amino acid sequence 100% identical to the amino acid sequence of SEQ ID NO: 189, and a CDR H3 having an amino acid sequence 100% identical to the amino acid sequence of SEQ ID NO: 190; and a VL comprising therein a complementarity determining region (CDR) L1 having an amino acid sequence 100% identical to the amino acid sequence of SEQ ID NO: 192, a CDR L2 having an amino acid sequence 100% identical to the amino acid sequence of DAT, and a CDR L3 having an amino acid sequence 100% identical to the amino acid sequence of SEQ ID NO: 193.

207. The monospecific antibody of any one of claims 1-11 and 201-206, wherein the antibody comprises: (a) a variable heavy chain (VH) region having an amino acid sequence with at least 80% identity to SEQ ID NO: 187 and comprising therein a complementarity determining region (CDR) H1 having an amino acid sequence 100% identical to the amino acid sequence of SEQ ID NO: 188, a CDR H2 having an amino acid sequence 100% identical to the amino acid sequence of SEQ ID NO: 189, and a CDR H3 having an amino acid sequence 100% identical to the amino acid sequence of SEQ ID NO: 190; a variable light chain (VL) region having an amino acid sequence with at least 80% identity to SEQ ID NO: 191 and comprising therein a complementarity determining region (CDR) L1 having an amino acid sequence 100% identical to the amino acid sequence of SEQ ID NO: 192, a CDR L2 having an amino acid sequence 100% identical to the amino acid sequence of DAT, and a CDR L3 having an amino acid sequence 100% identical to the amino acid sequence of SEQ ID NO: 193.

208. The monospecific antibody of any one of claims 1-11, wherein the antibody comprises a variable heavy chain (VH) region having an amino acid sequence with at least 80% identity to SEQ ID NO: 194 and a variable light chain (VL) region having an amino acid sequence with at least 80% identity to SEQ ID NO: 198.

209. The monospecific antibody of any one of claims 1-11 and 208, wherein the antibody comprises a variable heavy chain (VH) region having an amino acid sequence with at least 85% identity to SEQ ID NO: 194 and a variable light chain (VL) region having an amino acid sequence with at least 85% identity to SEQ ID NO: 198.

210. The monospecific antibody of any one of claims 1-11, 208, and 209, wherein the antibody comprises a variable heavy chain (VH) region having an amino acid sequence with at least 90% identity to SEQ ID NO: 194 and a variable light chain (VL) region having an amino acid sequence with at least 90% identity to SEQ ID NO: 198.

211. The monospecific antibody of any one of claims 1-11, and 208-210, wherein the antibody comprises a variable heavy chain (VH) region having an amino acid sequence with at least 95% identity to SEQ ID NO: 194 and a variable light chain (VL) region having an amino acid sequence with at least 95% identity to SEQ ID NO: 198.

212. The monospecific antibody of any one of claims 1-11 and 208-211, wherein the antibody comprises a variable heavy chain (VH) region having an amino acid sequence 100% identical to the amino acid sequence of SEQ ID NO: 194 and a variable light chain (VL) region having an amino acid sequence 100% identical to the amino acid sequence of SEQ ID NO: 198.

213. The monospecific antibody of any one of claims 1-11 and 208-212, wherein the antibody comprises a VH comprising therein a complementarity determining region (CDR) H1 having an amino acid sequence 100% identical to the amino acid sequence of SEQ ID NO: 195, a CDR H2 having an amino acid sequence 100% identical to the amino acid sequence of SEQ ID NO: 196, and a CDR H3 having an amino acid sequence 100% identical to the amino acid sequence of SEQ ID NO: 197; and a VL comprising therein a complementarity determining region (CDR) L1 having an amino acid sequence 100% identical to the amino acid sequence of SEQ ID NO: 199, a CDR L2 having an amino acid sequence 100% identical to the amino acid sequence of DAS, and a CDR L3 having an amino acid sequence 100% identical to the amino acid sequence of SEQ ID NO: 200.

214. The monospecific antibody of any one of claims 1-11 and 208-213, wherein the antibody comprises: (a) a variable heavy chain (VH) region having an amino acid sequence with at least 80% identity to SEQ ID NO: 194 and comprising therein a complementarity determining region (CDR) H1 having an amino acid sequence 100% identical to the amino acid sequence of SEQ ID NO: 195, a CDR H2 having an amino acid sequence 100% identical to the amino acid sequence of SEQ ID NO: 196, and a CDR H3 having an amino acid sequence 100% identical to the amino acid sequence of SEQ ID NO: 197; a variable light chain (VL) region having an amino acid sequence with at least 80% identity to SEQ ID NO: 198 and comprising therein a complementarity determining region (CDR) L1 having an amino acid sequence 100% identical to the amino acid sequence of SEQ ID NO: 199, a CDR L2 having an amino acid sequence 100% identical to the amino acid sequence of DAS, and a CDR L3 having an amino acid sequence 100% identical to the amino acid sequence of SEQ ID NO: 200.

215. The monospecific antibody of any one of claims 1-11, wherein the antibody comprises a variable heavy chain (VH) region having an amino acid sequence with at least 80% identity to SEQ ID NO: 201 and a variable light chain (VL) region having an amino acid sequence with at least 80% identity to SEQ ID NO: 205.

216. The monospecific antibody of any one of claims 1-11 and 215, wherein the antibody comprises a variable heavy chain (VH) region having an amino acid sequence with at least 85% identity to SEQ ID NO: 201 and a variable light chain (VL) region having an amino acid sequence with at least 85% identity to SEQ ID NO: 205.

217. The monospecific antibody of any one of claims 1-11, 215, and 216, wherein the antibody comprises a variable heavy chain (VH) region having an amino acid sequence with at least 90% identity to SEQ ID NO: 201 and a variable light chain (VL) region having an amino acid sequence with at least 90% identity to SEQ ID NO: 205.

218. The monospecific antibody of any one of claims 1-11 and 215-217, wherein the antibody comprises a variable heavy chain (VH) region having an amino acid sequence with at least 95% identity to SEQ ID NO: 201 and a variable light chain (VL) region having an amino acid sequence with at least 95% identity to SEQ ID NO: 205.

219. The monospecific antibody of any one of claims 1-11 and 215-218, wherein the antibody comprises a variable heavy chain (VH) region having an amino acid sequence 100% identical to the amino acid sequence of SEQ ID NO: 201 and a variable light chain (VL) region having an amino acid sequence 100% identical to the amino acid sequence of SEQ ID NO: 205.

220. The monospecific antibody of any one of claims 1-11 and 215-219, wherein the antibody comprises a VH comprising therein a complementarity determining region (CDR) H1 having an amino acid sequence 100% identical to the amino acid sequence of SEQ ID NO: 202, a CDR H2 having an amino acid sequence 100% identical to the amino acid sequence of SEQ ID NO: 203, and a CDR H3 having an amino acid sequence 100% identical to the amino acid sequence of SEQ ID NO: 204; and a VL comprising therein a complementarity determining region (CDR) L1 having an amino acid sequence 100% identical to the amino acid sequence of SEQ ID NO: 206, a CDR L2 having an amino acid sequence 100% identical to the amino acid sequence of GVD, and a CDR L3 having an amino acid sequence 100% identical to the amino acid sequence of SEQ ID NO: 207.

221. The monospecific antibody of any one of claims 1-11 and 215-220, wherein the antibody comprises: (a) a variable heavy chain (VH) region having an amino acid sequence with at least 80% identity to SEQ ID NO: 201 and comprising therein a complementarity determining region (CDR) H1 having an amino acid sequence 100% identical to the amino acid sequence of SEQ ID NO: 202, a CDR H2 having an amino acid sequence 100% identical to the amino acid sequence of SEQ ID NO: 203, and a CDR H3 having an amino acid sequence 100% identical to the amino acid sequence of SEQ ID NO: 204; a variable light chain (VL) region having an amino acid sequence with at least 80% identity to SEQ ID NO: 205 and comprising therein a complementarity determining region (CDR) L1 having an amino acid sequence 100% identical to the amino acid sequence of SEQ ID NO: 206, a CDR L2 having an amino acid sequence 100% identical to the amino acid sequence of GVD, and a CDR L3 having an amino acid sequence 100% identical to the amino acid sequence of SEQ ID NO: 207.

222. The monospecific antibody of any one of claims 1-11, wherein the antibody comprises a variable heavy chain (VH) region having an amino acid sequence with at least 80% identity to SEQ ID NO: 208 and a variable light chain (VL) region having an amino acid sequence with at least 80% identity to SEQ ID NO: 212.

223. The monospecific antibody of any one of claims 1-11 and 222, wherein the antibody comprises a variable heavy chain (VH) region having an amino acid sequence with at least 85% identity to SEQ ID NO: 208 and a variable light chain (VL) region having an amino acid sequence with at least 85% identity to SEQ ID NO: 212.

224. The monospecific antibody of any one of claims 1-11, 222, and 223, wherein the antibody comprises a variable heavy chain (VH) region having an amino acid sequence with at least 90% identity to SEQ ID NO: 208 and a variable light chain (VL) region having an amino acid sequence with at least 90% identity to SEQ ID NO: 212.

225. The monospecific antibody of any one of claims 1-11 and 222-224, wherein the antibody comprises a variable heavy chain (VH) region having an amino acid sequence with at least 95% identity to SEQ ID NO: 208 and a variable light chain (VL) region having an amino acid sequence with at least 95% identity to SEQ ID NO: 212.

226. The monospecific antibody of any one of claims 1-11 and 222-225, wherein the antibody comprises a variable heavy chain (VH) region having an amino acid sequence 100% identical to the amino acid sequence of SEQ ID NO: 208 and a variable light chain (VL) region having an amino acid sequence 100% identical to the amino acid sequence of SEQ ID NO: 212.

227. The monospecific antibody of any one of claims 1-11 and 222-226, wherein the antibody comprises a VH comprising therein a complementarity determining region (CDR) H1 having an amino acid sequence 100% identical to the amino acid sequence of SEQ ID NO: 209, a CDR H2 having an amino acid sequence 100% identical to the amino acid sequence of SEQ ID NO: 210, and a CDR H3 having an amino acid sequence 100% identical to the amino acid sequence of SEQ ID NO: 211; and a VL comprising therein a complementarity determining region (CDR) L1 having an amino acid sequence 100% identical to the amino acid sequence of SEQ ID NO: 212, a CDR L2 having an amino acid sequence 100% identical to the amino acid sequence of DAS, and a CDR L3 having an amino acid sequence 100% identical to the amino acid sequence of SEQ ID NO: 213.

228. The monospecific antibody of any one of claims 1-11 and 222-227, wherein the antibody comprises: (a) a variable heavy chain (VH) region having an amino acid sequence with at least 80% identity to SEQ ID NO: 208 and comprising therein a complementarity determining region (CDR) H1 having an amino acid sequence 100% identical to the amino acid sequence of SEQ ID NO: 209, a CDR H2 having an amino acid sequence 100% identical to the amino acid sequence of SEQ ID NO: 210, and a CDR H3 having an amino acid sequence 100% identical to the amino acid sequence of SEQ ID NO: 211; a variable light chain (VL) region having an amino acid sequence with at least 80% identity to SEQ ID NO: 212 and comprising therein a complementarity determining region (CDR) L1 having an amino acid sequence 100% identical to the amino acid sequence of SEQ ID NO: 212, a CDR L2 having an amino acid sequence 100% identical to the amino acid sequence of DAS, and a CDR L3 having an amino acid sequence 100% identical to the amino acid sequence of SEQ ID NO: 213.

229. The monospecific antibody of any one of claims 1-11, wherein the antibody comprises a variable heavy chain (VH) region having an amino acid sequence with at least 80% identity to SEQ ID NO: 215 and a variable light chain (VL) region having an amino acid sequence with at least 80% identity to SEQ ID NO: 219.

230. The monospecific antibody of any one of claims 1-11 and 229, wherein the antibody comprises a variable heavy chain (VH) region having an amino acid sequence with at least 85% identity to SEQ ID NO: 215 and a variable light chain (VL) region having an amino acid sequence with at least 85% identity to SEQ ID NO: 219.

231. The monospecific antibody of any one of claims 1-11, 229, and 203, wherein the antibody comprises a variable heavy chain (VH) region having an amino acid sequence with at least 90% identity to SEQ ID NO: 215 and a variable light chain (VL) region having an amino acid sequence with at least 90% identity to SEQ ID NO: 219.

232. The monospecific antibody of any one of claims 1-11 and 229-231, wherein the antibody comprises a variable heavy chain (VH) region having an amino acid sequence with at least 95% identity to SEQ ID NO: 215 and a variable light chain (VL) region having an amino acid sequence with at least 95% identity to SEQ ID NO: 219.

233. The monospecific antibody of any one of claims 1-11 and 229-232, wherein the antibody comprises a variable heavy chain (VH) region having an amino acid sequence 100% identical to the amino acid sequence of SEQ ID NO: 215 and a variable light chain (VL) region having an amino acid sequence 100% identical to the amino acid sequence of SEQ ID NO: 219.

234. The monospecific antibody of any one of claims 1-11 and 229-233, wherein the antibody comprises a VH comprising therein a complementarity determining region (CDR) H1 having an amino acid sequence 100% identical to the amino acid sequence of SEQ ID NO: 216, a CDR H2 having an amino acid sequence 100% identical to the amino acid sequence of SEQ ID NO: 217, and a CDR H3 having an amino acid sequence 100% identical to the amino acid sequence of SEQ ID NO: 218; and a VL comprising therein a complementarity determining region (CDR) L1 having an amino acid sequence 100% identical to the amino acid sequence of SEQ ID NO: 220, a CDR L2 having an amino acid sequence 100% identical to the amino acid sequence of GAT, and a CDR L3 having an amino acid sequence 100% identical to the amino acid sequence of SEQ ID NO: 221.

235. The monospecific antibody of any one of claims 1-11 and 229-234, wherein the antibody comprises: (a) a variable heavy chain (VH) region having an amino acid sequence with at least 80% identity to SEQ ID NO: 215 and comprising therein a complementarity determining region (CDR) H1 having an amino acid sequence 100% identical to the amino acid sequence of SEQ ID NO: 216, a CDR H2 having an amino acid sequence 100% identical to the amino acid sequence of SEQ ID NO: 217, and a CDR H3 having an amino acid sequence 100% identical to the amino acid sequence of SEQ ID NO: 218; a variable light chain (VL) region having an amino acid sequence with at least 80% identity to SEQ ID NO: 219 and comprising therein a complementarity determining region (CDR) L1 having an amino acid sequence 100% identical to the amino acid sequence of SEQ ID NO: 220, a CDR L2 having an amino acid sequence 100% identical to the amino acid sequence of GAT, and a CDR L3 having an amino acid sequence 100% identical to the amino acid sequence of SEQ ID NO: 221.

236. The monospecific antibody of any one of claims 1-11, wherein the antibody comprises a variable heavy chain (VH) region having an amino acid sequence with at least 80% identity to SEQ ID NO: 222 and a variable light chain (VL) region having an amino acid sequence with at least 80% identity to SEQ ID NO: 226.

237. The monospecific antibody of any one of claims 1-11 and 236, wherein the antibody comprises a variable heavy chain (VH) region having an amino acid sequence with at least 85% identity to SEQ ID NO: 222 and a variable light chain (VL) region having an amino acid sequence with at least 85% identity to SEQ ID NO: 226.

238. The monospecific antibody of any one of claims 1-11, 236, and 237, wherein the antibody comprises a variable heavy chain (VH) region having an amino acid sequence with at least 90% identity to SEQ ID NO: 222 and a variable light chain (VL) region having an amino acid sequence with at least 90% identity to SEQ ID NO: 226.

239. The monospecific antibody of any one of claims 1-11 and 236-238, wherein the antibody comprises a variable heavy chain (VH) region having an amino acid sequence with at least 95% identity to SEQ ID NO: 222 and a variable light chain (VL) region having an amino acid sequence with at least 95% identity to SEQ ID NO: 226.

240. The monospecific antibody of any one of claims 1-11 and 236-239, wherein the antibody comprises a variable heavy chain (VH) region having an amino acid sequence 100% identical to the amino acid sequence of SEQ ID NO: 222 and a variable light chain (VL) region having an amino acid sequence 100% identical to the amino acid sequence of SEQ ID NO: 226.

241. The monospecific antibody of any one of claims 1-11 and 236-240, wherein the antibody comprises a VH comprising therein a complementarity determining region (CDR) H1 having an amino acid sequence 100% identical to the amino acid sequence of SEQ ID NO: 223, a CDR H2 having an amino acid sequence 100% identical to the amino acid sequence of SEQ ID NO: 224, and a CDR H3 having an amino acid sequence 100% identical to the amino acid sequence of SEQ ID NO: 225; and a VL comprising therein a complementarity determining region (CDR) L1 having an amino acid sequence 100% identical to the amino acid sequence of SEQ ID NO: 227, a CDR L2 having an amino acid sequence 100% identical to the amino acid sequence of DAS, and a CDR L3 having an amino acid sequence 100% identical to the amino acid sequence of SEQ ID NO: 228.

242. The monospecific antibody of any one of claims 1-11 and 236-241, wherein the antibody comprises: (a) a variable heavy chain (VH) region having an amino acid sequence with at least 80% identity to SEQ ID NO: 222 and comprising therein a complementarity determining region (CDR) H1 having an amino acid sequence 100% identical to the amino acid sequence of SEQ ID NO: 223, a CDR H2 having an amino acid sequence 100% identical to the amino acid sequence of SEQ ID NO: 224, and a CDR H3 having an amino acid sequence 100% identical to the amino acid sequence of SEQ ID NO: 225; a variable light chain (VL) region having an amino acid sequence with at least 80% identity to SEQ ID NO: 226 and comprising therein a complementarity determining region (CDR) L1 having an amino acid sequence 100% identical to the amino acid sequence of SEQ ID NO: 227, a CDR L2 having an amino acid sequence 100% identical to the amino acid sequence of DAS, and a CDR L3 having an amino acid sequence 100% identical to the amino acid sequence of SEQ ID NO: 228.

243. The monospecific antibody of any one of claims 1-11, wherein the antibody comprises a variable heavy chain (VH) region having an amino acid sequence with at least 80% identity to SEQ ID NO: 229 and a variable light chain (VL) region having an amino acid sequence with at least 80% identity to SEQ ID NO: 233.

244. The monospecific antibody of any one of claims 1-11 and 243, wherein the antibody comprises a variable heavy chain (VH) region having an amino acid sequence with at least 85% identity to SEQ ID NO: 229 and a variable light chain (VL) region having an amino acid sequence with at least 85% identity to SEQ ID NO: 233.

245. The monospecific antibody of any one of claims 1-11, 243, and 244, wherein the antibody comprises a variable heavy chain (VH) region having an amino acid sequence with at least 90% identity to SEQ ID NO: 229 and a variable light chain (VL) region having an amino acid sequence with at least 90% identity to SEQ ID NO: 233.

246. The monospecific antibody of any one of claims 1-11 and 243-245, wherein the antibody comprises a variable heavy chain (VH) region having an amino acid sequence with at least 95% identity to SEQ ID NO: 229 and a variable light chain (VL) region having an amino acid sequence with at least 95% identity to SEQ ID NO: 233.

247. The monospecific antibody of any one of claims 1-11 and 243-246, wherein the antibody comprises a variable heavy chain (VH) region having an amino acid sequence 100% identical to the amino acid sequence of SEQ ID NO: 229 and a variable light chain (VL) region having an amino acid sequence 100% identical to the amino acid sequence of SEQ ID NO: 233.

248. The monospecific antibody of any one of claims 1-11 and 243-247, wherein the antibody comprises a VH comprising therein a complementarity determining region (CDR) H1 having an amino acid sequence 100% identical to the amino acid sequence of SEQ ID NO: 230, a CDR H2 having an amino acid sequence 100% identical to the amino acid sequence of SEQ ID NO: 231, and a CDR H3 having an amino acid sequence 100% identical to the amino acid sequence of SEQ ID NO: 232; and a VL comprising therein a complementarity determining region (CDR) L1 having an amino acid sequence 100% identical to the amino acid sequence of SEQ ID NO: 234, a CDR L2 having an amino acid sequence 100% identical to the amino acid sequence of DAS, and a CDR L3 having an amino acid sequence 100% identical to the amino acid sequence of SEQ ID NO: 235.

249. The monospecific antibody of any one of claims 1-11 and 243-248, wherein the antibody comprises: (a) a variable heavy chain (VH) region having an amino acid sequence with at least 80% identity to SEQ ID NO: 229 and comprising therein a complementarity determining region (CDR) H1 having an amino acid sequence 100% identical to the amino acid sequence of SEQ ID NO: 230, a CDR H2 having an amino acid sequence 100% identical to the amino acid sequence of SEQ ID NO: 231, and a CDR H3 having an amino acid sequence 100% identical to the amino acid sequence of SEQ ID NO: 232; a variable light chain (VL) region having an amino acid sequence with at least 80% identity to SEQ ID NO: 233 and comprising therein a complementarity determining region (CDR) L1 having an amino acid sequence 100% identical to the amino acid sequence of SEQ ID NO: 234, a CDR L2 having an amino acid sequence 100% identical to the amino acid sequence of DAS, and a CDR L3 having an amino acid sequence 100% identical to the amino acid sequence of SEQ ID NO: 235.

250. The monospecific antibody of any one of claims 1-11, wherein the antibody comprises a variable heavy chain (VH) region having an amino acid sequence with at least 80% identity to SEQ ID NO: 236 and a variable light chain (VL) region having an amino acid sequence with at least 80% identity to SEQ ID NO: 240.

251. The monospecific antibody of any one of claims 1-11 and 250, wherein the antibody comprises a variable heavy chain (VH) region having an amino acid sequence with at least 85% identity to SEQ ID NO: 236 and a variable light chain (VL) region having an amino acid sequence with at least 85% identity to SEQ ID NO: 240.

252. The monospecific antibody of any one of claims 1-11, 250, and 251, wherein the antibody comprises a variable heavy chain (VH) region having an amino acid sequence with at least 90% identity to SEQ ID NO: 236 and a variable light chain (VL) region having an amino acid sequence with at least 90% identity to SEQ ID NO: 240.

253. The monospecific antibody of any one of claims 1-11 and 250-252, wherein the antibody comprises a variable heavy chain (VH) region having an amino acid sequence with at least 95% identity to SEQ ID NO: 236 and a variable light chain (VL) region having an amino acid sequence with at least 95% identity to SEQ ID NO: 240.

254. The monospecific antibody of any one of claims 1-11 and 250-253, wherein the antibody comprises a variable heavy chain (VH) region having an amino acid sequence 100% identical to the amino acid sequence of SEQ ID NO: 236 and a variable light chain (VL) region having an amino acid sequence 100% identical to the amino acid sequence of SEQ ID NO: 240.

255. The monospecific antibody of any one of claims 1-11 and 250-254, wherein the antibody comprises a VH comprising therein a complementarity determining region (CDR) H1 having an amino acid sequence 100% identical to the amino acid sequence of SEQ ID NO: 237, a CDR H2 having an amino acid sequence 100% identical to the amino acid sequence of SEQ ID NO: 238, and a CDR H3 having an amino acid sequence 100% identical to the amino acid sequence of SEQ ID NO: 239; and a VL comprising therein a complementarity determining region (CDR) L1 having an amino acid sequence 100% identical to the amino acid sequence of SEQ ID NO: 241, a CDR L2 having an amino acid sequence 100% identical to the amino acid sequence of GAS, and a CDR L3 having an amino acid sequence 100% identical to the amino acid sequence of SEQ ID NO: 242.

256. The monospecific antibody of any one of claims 1-11 and 250-255, wherein the antibody comprises: (a) a variable heavy chain (VH) region having an amino acid sequence with at least 80% identity to SEQ ID NO: 236 and comprising therein a complementarity determining region (CDR) H1 having an amino acid sequence 100% identical to the amino acid sequence of SEQ ID NO: 237, a CDR H2 having an amino acid sequence 100% identical to the amino acid sequence of SEQ ID NO: 238, and a CDR H3 having an amino acid sequence 100% identical to the amino acid sequence of SEQ ID NO: 239; a variable light chain (VL) region having an amino acid sequence with at least 80% identity to SEQ ID NO: 240 and comprising therein a complementarity determining region (CDR) L1 having an amino acid sequence 100% identical to the amino acid sequence of SEQ ID NO: 241, a CDR L2 having an amino acid sequence 100% identical to the amino acid sequence of GAS, and a CDR L3 having an amino acid sequence 100% identical to the amino acid sequence of SEQ ID NO: 242.

257. The monospecific antibody of any one of claims 1-11, wherein the antibody comprises a variable heavy chain (VH) region having an amino acid sequence with at least 80% identity to SEQ ID NO: 243 and a variable light chain (VL) region having an amino acid sequence with at least 80% identity to SEQ ID NO: 247.

258. The monospecific antibody of any one of claims 1-11 and 257, wherein the antibody comprises a variable heavy chain (VH) region having an amino acid sequence with at least 85% identity to SEQ ID NO: 243 and a variable light chain (VL) region having an amino acid sequence with at least 85% identity to SEQ ID NO: 247.

259. The monospecific antibody of any one of claims 1-11, 257, and 258, wherein the antibody comprises a variable heavy chain (VH) region having an amino acid sequence with at least 90% identity to SEQ ID NO: 243 and a variable light chain (VL) region having an amino acid sequence with at least 90% identity to SEQ ID NO: 247.

260. The monospecific antibody of any one of claims 1-11 and 257-259, wherein the antibody comprises a variable heavy chain (VH) region having an amino acid sequence with at least 95% identity to SEQ ID NO: 243 and a variable light chain (VL) region having an amino acid sequence with at least 95% identity to SEQ ID NO: 247.

261. The monospecific antibody of any one of claims 1-11 and 257-260, wherein the antibody comprises a variable heavy chain (VH) region having an amino acid sequence 100% identical to the amino acid sequence of SEQ ID NO: 243 and a variable light chain (VL) region having an amino acid sequence 100% identical to the amino acid sequence of SEQ ID NO: 247.

262. The monospecific antibody of any one of claims 1-11 and 257-261, wherein the antibody comprises a VH comprising therein a complementarity determining region (CDR) H1 having an amino acid sequence 100% identical to the amino acid sequence of SEQ ID NO: 244, a CDR H2 having an amino acid sequence 100% identical to the amino acid sequence of SEQ ID NO: 245, and a CDR H3 having an amino acid sequence 100% identical to the amino acid sequence of SEQ ID NO: 246; and a VL comprising therein a complementarity determining region (CDR) L1 having an amino acid sequence 100% identical to the amino acid sequence of SEQ ID NO: 248, a CDR L2 having an amino acid sequence 100% identical to the amino acid sequence of GAS, and a CDR L3 having an amino acid sequence 100% identical to the amino acid sequence of SEQ ID NO: 249.

263. The monospecific antibody of any one of claims 1-11 and 257-262, wherein the antibody comprises: (a) a variable heavy chain (VH) region having an amino acid sequence with at least 80% identity to SEQ ID NO: 243 and comprising therein a complementarity determining region (CDR) H1 having an amino acid sequence 100% identical to the amino acid sequence of SEQ ID NO: 244, a CDR H2 having an amino acid sequence 100% identical to the amino acid sequence of SEQ ID NO: 245, and a CDR H3 having an amino acid sequence 100% identical to the amino acid sequence of SEQ ID NO: 246; a variable light chain (VL) region having an amino acid sequence with at least 80% identity to SEQ ID NO: 247 and comprising therein a complementarity determining region (CDR) L1 having an amino acid sequence 100% identical to the amino acid sequence of SEQ ID NO: 248, a CDR L2 having an amino acid sequence 100% identical to the amino acid sequence of GAS, and a CDR L3 having an amino acid sequence 100% identical to the amino acid sequence of SEQ ID NO: 249.

264. The monospecific antibody of any one of claims 1-11 and 257-263, wherein the antibody comprises a first heavy chain (HC) region comprising an amino acid sequence having at least 80% identity to SEQ ID NO: 460, a second heavy chain region comprising an amino acid sequence having at least 80% identity to SEQ ID NO: 480, and a light chain (LC) region, comprising an amino acid sequence having at least 80% identity to SEQ ID NO: 470.

265. The monospecific antibody of any one of claims 1-11, wherein the antibody comprises a variable heavy chain (VH) region having an amino acid sequence with at least 80% identity to SEQ ID NO: 250 and a variable light chain (VL) region having an amino acid sequence with at least 80% identity to SEQ ID NO: 254.

266. The monospecific antibody of any one of claims 1-11 and 265, wherein the antibody comprises a variable heavy chain (VH) region having an amino acid sequence with at least 85% identity to SEQ ID NO: 250 and a variable light chain (VL) region having an amino acid sequence with at least 85% identity to SEQ ID NO: 254.

267. The monospecific antibody of any one of claims 1-11, 265, and 266, wherein the antibody comprises a variable heavy chain (VH) region having an amino acid sequence with at least 90% identity to SEQ ID NO: 250 and a variable light chain (VL) region having an amino acid sequence with at least 90% identity to SEQ ID NO: 254.

268. The monospecific antibody of any one of claims 1-11 and 265-267, wherein the antibody comprises a variable heavy chain (VH) region having an amino acid sequence with at least 95% identity to SEQ ID NO: 250 and a variable light chain (VL) region having an amino acid sequence with at least 95% identity to SEQ ID NO: 254.

269. The monospecific antibody of any one of claims 1-11 and 265-268, wherein the antibody comprises a variable heavy chain (VH) region having an amino acid sequence 100% identical to the amino acid sequence of SEQ ID NO: 250 and a variable light chain (VL) region having an amino acid sequence 100% identical to the amino acid sequence of SEQ ID NO: 254.

270. The monospecific antibody of any one of claims 1-11 and 265-269, wherein the antibody comprises a VH comprising therein a complementarity determining region (CDR) H1 having an amino acid sequence 100% identical to the amino acid sequence of SEQ ID NO: 251, a CDR H2 having an amino acid sequence 100% identical to the amino acid sequence of SEQ ID NO: 252, and a CDR H3 having an amino acid sequence 100% identical to the amino acid sequence of SEQ ID NO: 253; and a VL comprising therein a complementarity determining region (CDR) L1 having an amino acid sequence 100% identical to the amino acid sequence of SEQ ID NO: 255, a CDR L2 having an amino acid sequence 100% identical to the amino acid sequence of GAT, and a CDR L3 having an amino acid sequence 100% identical to the amino acid sequence of SEQ ID NO: 256.

271. The monospecific antibody of any one of claims 1-11 and 265-270, wherein the antibody comprises: (a) a variable heavy chain (VH) region having an amino acid sequence with at least 80% identity to SEQ ID NO: 250 and comprising therein a complementarity determining region (CDR) H1 having an amino acid sequence 100% identical to the amino acid sequence of SEQ ID NO: 251, a CDR H2 having an amino acid sequence 100% identical to the amino acid sequence of SEQ ID NO: 252, and a CDR H3 having an amino acid sequence 100% identical to the amino acid sequence of SEQ ID NO: 253; a variable light chain (VL) region having an amino acid sequence with at least 80% identity to SEQ ID NO: 254 and comprising therein a complementarity determining region (CDR) L1 having an amino acid sequence 100% identical to the amino acid sequence of SEQ ID NO: 255, a CDR L2 having an amino acid sequence 100% identical to the amino acid sequence of GAT, and a CDR L3 having an amino acid sequence 100% identical to the amino acid sequence of SEQ ID NO: 256.

272. The monospecific antibody of any one of claims 1-11, wherein the antibody comprises a variable heavy chain (VH) region having an amino acid sequence with at least 80% identity to SEQ ID NO: 257 and a variable light chain (VL) region having an amino acid sequence with at least 80% identity to SEQ ID NO: 261.

273. The monospecific antibody of any one of claims 1-11 and 272, wherein the antibody comprises a variable heavy chain (VH) region having an amino acid sequence with at least 85% identity to SEQ ID NO: 257 and a variable light chain (VL) region having an amino acid sequence with at least 85% identity to SEQ ID NO: 261.

274. The monospecific antibody of any one of claims 1-11, 272, and 273, wherein the antibody comprises a variable heavy chain (VH) region having an amino acid sequence with at least 90% identity to SEQ ID NO: 257 and a variable light chain (VL) region having an amino acid sequence with at least 90% identity to SEQ ID NO: 261.

275. The monospecific antibody of any one of claims 1-11 and 272-274, wherein the antibody comprises a variable heavy chain (VH) region having an amino acid sequence with at least 95% identity to SEQ ID NO: 257 and a variable light chain (VL) region having an amino acid sequence with at least 95% identity to SEQ ID NO: 261.

276. The monospecific antibody of any one of claims 1-11 and 272-275, wherein the antibody comprises a variable heavy chain (VH) region having an amino acid sequence 100% identical to the amino acid sequence of SEQ ID NO: 257 and a variable light chain (VL) region having an amino acid sequence 100% identical to the amino acid sequence of SEQ ID NO: 261.

277. The monospecific antibody of any one of claims 1-11 and 272-276, wherein the antibody comprises a VH comprising therein a complementarity determining region (CDR) H1 having an amino acid sequence 100% identical to the amino acid sequence of SEQ ID NO: 258, a CDR H2 having an amino acid sequence 100% identical to the amino acid sequence of SEQ ID NO: 259, and a CDR H3 having an amino acid sequence 100% identical to the amino acid sequence of SEQ ID NO: 260; and a VL comprising therein a complementarity determining region (CDR) L1 having an amino acid sequence 100% identical to the amino acid sequence of SEQ ID NO: 262, a CDR L2 having an amino acid sequence 100% identical to the amino acid sequence of GAS, and a CDR L3 having an amino acid sequence 100% identical to the amino acid sequence of SEQ ID NO: 263.

278. The monospecific antibody of any one of claims 1-11 and 272-277, wherein the antibody comprises: (a) a variable heavy chain (VH) region having an amino acid sequence with at least 80% identity to SEQ ID NO: 257 and comprising therein a complementarity determining region (CDR) H1 having an amino acid sequence 100% identical to the amino acid sequence of SEQ ID NO: 258, a CDR H2 having an amino acid sequence 100% identical to the amino acid sequence of SEQ ID NO: 259, and a CDR H3 having an amino acid sequence 100% identical to the amino acid sequence of SEQ ID NO: 260; a variable light chain (VL) region having an amino acid sequence with at least 80% identity to SEQ ID NO: 261 and comprising therein a complementarity determining region (CDR) L1 having an amino acid sequence 100% identical to the amino acid sequence of SEQ ID NO: 262, a CDR L2 having an amino acid sequence 100% identical to the amino acid sequence of GAS, and a CDR L3 having an amino acid sequence 100% identical to the amino acid sequence of SEQ ID NO: 263.

279. The monospecific antibody of any one of claims 1-11, wherein the antibody comprises a variable heavy chain (VH) region having an amino acid sequence with at least 80% identity to SEQ ID NO: 264 and a variable light chain (VL) region having an amino acid sequence with at least 80% identity to SEQ ID NO: 268.

280. The monospecific antibody of any one of claims 1-11 and 279, wherein the antibody comprises a variable heavy chain (VH) region having an amino acid sequence with at least 85% identity to SEQ ID NO: 264 and a variable light chain (VL) region having an amino acid sequence with at least 85% identity to SEQ ID NO: 268.

281. The monospecific antibody of any one of claims 1-11, 279, and 280, wherein the antibody comprises a variable heavy chain (VH) region having an amino acid sequence with at least 90% identity to SEQ ID NO: 264 and a variable light chain (VL) region having an amino acid sequence with at least 90% identity to SEQ ID NO: 268.

282. The monospecific antibody of any one of claims 1-11 and 279-281, wherein the antibody comprises a variable heavy chain (VH) region having an amino acid sequence with at least 95% identity to SEQ ID NO: 264 and a variable light chain (VL) region having an amino acid sequence with at least 95% identity to SEQ ID NO: 268.

283. The monospecific antibody of any one of claims 1-11 and 279-282, wherein the antibody comprises a variable heavy chain (VH) region having an amino acid sequence 100% identical to the amino acid sequence of SEQ ID NO: 264 and a variable light chain (VL) region having an amino acid sequence 100% identical to the amino acid sequence of SEQ ID NO: 268.

284. The monospecific antibody of any one of claims 1-11 and 279-283, wherein the antibody comprises a VH comprising therein a complementarity determining region (CDR) H1 having an amino acid sequence 100% identical to the amino acid sequence of SEQ ID NO: 265, a CDR H2 having an amino acid sequence 100% identical to the amino acid sequence of SEQ ID NO: 266, and a CDR H3 having an amino acid sequence 100% identical to the amino acid sequence of SEQ ID NO: 267; and a VL comprising therein a complementarity determining region (CDR) L1 having an amino acid sequence 100% identical to the amino acid sequence of SEQ ID NO: 269, a CDR L2 having an amino acid sequence 100% identical to the amino acid sequence of DAV, and a CDR L3 having an amino acid sequence 100% identical to the amino acid sequence of SEQ ID NO: 270.

285. The monospecific antibody of any one of claims 1-11 and 279-284, wherein the antibody comprises: (a) a variable heavy chain (VH) region having an amino acid sequence with at least 80% identity to SEQ ID NO: 264 and comprising therein a complementarity determining region (CDR) H1 having an amino acid sequence 100% identical to the amino acid sequence of SEQ ID NO: 265, a CDR H2 having an amino acid sequence 100% identical to the amino acid sequence of SEQ ID NO: 266, and a CDR H3 having an amino acid sequence 100% identical to the amino acid sequence of SEQ ID NO: 267; a variable light chain (VL) region having an amino acid sequence with at least 80% identity to SEQ ID NO: 268 and comprising therein a complementarity determining region (CDR) L1 having an amino acid sequence 100% identical to the amino acid sequence of SEQ ID NO: 269, a CDR L2 having an amino acid sequence 100% identical to the amino acid sequence of DAV, and a CDR L3 having an amino acid sequence 100% identical to the amino acid sequence of SEQ ID NO: 270.

286. The monospecific antibody of any one of claims 1-11, wherein the antibody comprises a variable heavy chain (VH) region having an amino acid sequence with at least 80% identity to SEQ ID NO: 271 and a variable light chain (VL) region having an amino acid sequence with at least 80% identity to SEQ ID NO: 275.

287. The monospecific antibody of any one of claims 1-11 and 286, wherein the antibody comprises a variable heavy chain (VH) region having an amino acid sequence with at least 85% identity to SEQ ID NO: 271 and a variable light chain (VL) region having an amino acid sequence with at least 85% identity to SEQ ID NO: 275.

288. The monospecific antibody of any one of claims 1-11, 286, and 287, wherein the antibody comprises a variable heavy chain (VH) region having an amino acid sequence with at least 90% identity to SEQ ID NO: 271 and a variable light chain (VL) region having an amino acid sequence with at least 90% identity to SEQ ID NO: 275.

289. The monospecific antibody of any one of claims 1-11 and 286-288, wherein the antibody comprises a variable heavy chain (VH) region having an amino acid sequence with at least 95% identity to SEQ ID NO: 271 and a variable light chain (VL) region having an amino acid sequence with at least 95% identity to SEQ ID NO: 275.

290. The monospecific antibody of any one of claims 1-11 and 286-289, wherein the antibody comprises a variable heavy chain (VH) region having an amino acid sequence 100% identical to the amino acid sequence of SEQ ID NO: 271 and a variable light chain (VL) region having an amino acid sequence 100% identical to the amino acid sequence of SEQ ID NO: 275.

291. The monospecific antibody of any one of claims 1-11 and 286-290, wherein the antibody comprises a VH comprising therein a complementarity determining region (CDR) H1 having an amino acid sequence 100% identical to the amino acid sequence of SEQ ID NO: 272, a CDR H2 having an amino acid sequence 100% identical to the amino acid sequence of SEQ ID NO: 273, and a CDR H3 having an amino acid sequence 100% identical to the amino acid sequence of SEQ ID NO: 274; and a VL comprising therein a complementarity determining region (CDR) L1 having an amino acid sequence 100% identical to the amino acid sequence of SEQ ID NO: 276, a CDR L2 having an amino acid sequence 100% identical to the amino acid sequence of DAS, and a CDR L3 having an amino acid sequence 100% identical to the amino acid sequence of SEQ ID NO: 277.

292. The monospecific antibody of any one of claims 1-11 and 286-291, wherein the antibody comprises: (a) a variable heavy chain (VH) region having an amino acid sequence with at least 80% identity to SEQ ID NO: 271 and comprising therein a complementarity determining region (CDR) H1 having an amino acid sequence 100% identical to the amino acid sequence of SEQ ID NO: 272, a CDR H2 having an amino acid sequence 100% identical to the amino acid sequence of SEQ ID NO: 273, and a CDR H3 having an amino acid sequence 100% identical to the amino acid sequence of SEQ ID NO: 274; a variable light chain (VL) region having an amino acid sequence with at least 80% identity to SEQ ID NO: 275 and comprising therein a complementarity determining region (CDR) L1 having an amino acid sequence 100% identical to the amino acid sequence of SEQ ID NO: 276, a CDR L2 having an amino acid sequence 100% identical to the amino acid sequence of DAS, and a CDR L3 having an amino acid sequence 100% identical to the amino acid sequence of SEQ ID NO: 277.

293. The monospecific antibody of any one of claims 1-11, wherein the antibody comprises a variable heavy chain (VH) region having an amino acid sequence with at least 80% identity to SEQ ID NO: 278 and a variable light chain (VL) region having an amino acid sequence with at least 80% identity to SEQ ID NO: 282.

294. The monospecific antibody of any one of claims 1-11 and 293, wherein the antibody comprises a variable heavy chain (VH) region having an amino acid sequence with at least 85% identity to SEQ ID NO: 278 and a variable light chain (VL) region having an amino acid sequence with at least 85% identity to SEQ ID NO: 282.

295. The monospecific antibody of any one of claims 1-11, 293, and 294, wherein the antibody comprises a variable heavy chain (VH) region having an amino acid sequence with at least 90% identity to SEQ ID NO: 278 and a variable light chain (VL) region having an amino acid sequence with at least 90% identity to SEQ ID NO: 282.

296. The monospecific antibody of any one of claims 1-11 and 293-295, wherein the antibody comprises a variable heavy chain (VH) region having an amino acid sequence with at least 95% identity to SEQ ID NO: 278 and a variable light chain (VL) region having an amino acid sequence with at least 95% identity to SEQ ID NO: 282.

297. The monospecific antibody of any one of claims 1-11 and 293-296, wherein the antibody comprises a variable heavy chain (VH) region having an amino acid sequence 100% identical to the amino acid sequence of SEQ ID NO: 278 and a variable light chain (VL) region having an amino acid sequence 100% identical to the amino acid sequence of SEQ ID NO: 282.

298. The monospecific antibody of any one of claims 1-11 and 293-297, wherein the antibody comprises a VH comprising therein a complementarity determining region (CDR) H1 having an amino acid sequence 100% identical to the amino acid sequence of SEQ ID NO: 279, a CDR H2 having an amino acid sequence 100% identical to the amino acid sequence of SEQ ID NO: 280, and a CDR H3 having an amino acid sequence 100% identical to the amino acid sequence of SEQ ID NO: 281; and a VL comprising therein a complementarity determining region (CDR) L1 having an amino acid sequence 100% identical to the amino acid sequence of SEQ ID NO: 283, a CDR L2 having an amino acid sequence 100% identical to the amino acid sequence of DAS, and a CDR L3 having an amino acid sequence 100% identical to the amino acid sequence of SEQ ID NO: 284.

299. The monospecific antibody of any one of claims 1-11 and 293-298, wherein the antibody comprises: (a) a variable heavy chain (VH) region having an amino acid sequence with at least 80% identity to SEQ ID NO: 278 and comprising therein a complementarity determining region (CDR) H1 having an amino acid sequence 100% identical to the amino acid sequence of SEQ ID NO: 279, a CDR H2 having an amino acid sequence 100% identical to the amino acid sequence of SEQ ID NO: 280, and a CDR H3 having an amino acid sequence 100% identical to the amino acid sequence of SEQ ID NO: 281; a variable light chain (VL) region having an amino acid sequence with at least 80% identity to SEQ ID NO: 282 and comprising therein a complementarity determining region (CDR) L1 having an amino acid sequence 100% identical to the amino acid sequence of SEQ ID NO: 283, a CDR L2 having an amino acid sequence 100% identical to the amino acid sequence of DAS, and a CDR L3 having an amino acid sequence 100% identical to the amino acid sequence of SEQ ID NO: 284.

300. The monospecific antibody of any one of claims 1-11, wherein the antibody comprises a variable heavy chain (VH) region having an amino acid sequence with at least 80% identity to SEQ ID NO: 285 and a variable light chain (VL) region having an amino acid sequence with at least 80% identity to SEQ ID NO: 289.

301. The monospecific antibody of any one of claims 1-11 and 300, wherein the antibody comprises a variable heavy chain (VH) region having an amino acid sequence with at least 85% identity to SEQ ID NO: 285 and a variable light chain (VL) region having an amino acid sequence with at least 85% identity to SEQ ID NO: 289.

302. The monospecific antibody of any one of claims 1-11, 300, and 301, wherein the antibody comprises a variable heavy chain (VH) region having an amino acid sequence with at least 90% identity to SEQ ID NO: 285 and a variable light chain (VL) region having an amino acid sequence with at least 90% identity to SEQ ID NO: 289.

303. The monospecific antibody of any one of claims 1-11 and 300-302, wherein the antibody comprises a variable heavy chain (VH) region having an amino acid sequence with at least 95% identity to SEQ ID NO: 285 and a variable light chain (VL) region having an amino acid sequence with at least 95% identity to SEQ ID NO: 289.

304. The monospecific antibody of any one of claims 1-11 and 300-303, wherein the antibody comprises a variable heavy chain (VH) region having an amino acid sequence 100% identical to the amino acid sequence of SEQ ID NO: 285 and a variable light chain (VL) region having an amino acid sequence 100% identical to the amino acid sequence of SEQ ID NO: 289.

305. The monospecific antibody of any one of claims 1-11 and 300-304, wherein the antibody comprises a VH comprising therein a complementarity determining region (CDR) H1 having an amino acid sequence 100% identical to the amino acid sequence of SEQ ID NO: 286, a CDR H2 having an amino acid sequence 100% identical to the amino acid sequence of SEQ ID NO: 287, and a CDR H3 having an amino acid sequence 100% identical to the amino acid sequence of SEQ ID NO: 288; and a VL comprising therein a complementarity determining region (CDR) L1 having an amino acid sequence 100% identical to the amino acid sequence of SEQ ID NO: 290, a CDR L2 having an amino acid sequence 100% identical to the amino acid sequence of GAS, and a CDR L3 having an amino acid sequence 100% identical to the amino acid sequence of SEQ ID NO: 291.

306. The monospecific antibody of any one of claims 1-11 and 300-305, wherein the antibody comprises: (a) a variable heavy chain (VH) region having an amino acid sequence with at least 80% identity to SEQ ID NO: 285 and comprising therein a complementarity determining region (CDR) H1 having an amino acid sequence 100% identical to the amino acid sequence of SEQ ID NO: 286, a CDR H2 having an amino acid sequence 100% identical to the amino acid sequence of SEQ ID NO: 287, and a CDR H3 having an amino acid sequence 100% identical to the amino acid sequence of SEQ ID NO: 288; a variable light chain (VL) region having an amino acid sequence with at least 80% identity to SEQ ID NO: 289 and comprising therein a complementarity determining region (CDR) L1 having an amino acid sequence 100% identical to the amino acid sequence of SEQ ID NO: 290, a CDR L2 having an amino acid sequence 100% identical to the amino acid sequence of GAS, and a CDR L3 having an amino acid sequence 100% identical to the amino acid sequence of SEQ ID NO: 291.

307. The monospecific antibody of any one of claims 1-11, wherein the antibody comprises a variable heavy chain (VH) region having an amino acid sequence with at least 80% identity to SEQ ID NO: 292 and a variable light chain (VL) region having an amino acid sequence with at least 80% identity to SEQ ID NO: 296.

308. The monospecific antibody of any one of claims 1-11 and 307, wherein the antibody comprises a variable heavy chain (VH) region having an amino acid sequence with at least 85% identity to SEQ ID NO: 292 and a variable light chain (VL) region having an amino acid sequence with at least 85% identity to SEQ ID NO: 296.

309. The monospecific antibody of any one of claims 1-11, 307, and 308, wherein the antibody comprises a variable heavy chain (VH) region having an amino acid sequence with at least 90% identity to SEQ ID NO: 292 and a variable light chain (VL) region having an amino acid sequence with at least 90% identity to SEQ ID NO: 296.

310. The monospecific antibody of any one of claims 1-11 and 307-309, wherein the antibody comprises a variable heavy chain (VH) region having an amino acid sequence with at least 95% identity to SEQ ID NO: 292 and a variable light chain (VL) region having an amino acid sequence with at least 95% identity to SEQ ID NO: 296.

311. The monospecific antibody of any one of claims 1-11 and 307-310, wherein the antibody comprises a variable heavy chain (VH) region having an amino acid sequence 100% identical to the amino acid sequence of SEQ ID NO: 292 and a variable light chain (VL) region having an amino acid sequence 100% identical to the amino acid sequence of SEQ ID NO: 296.

312. The monospecific antibody of any one of claims 1-11 and 307-311, wherein the antibody comprises a VH comprising therein a complementarity determining region (CDR) H1 having an amino acid sequence 100% identical to the amino acid sequence of SEQ ID NO: 293, a CDR H2 having an amino acid sequence 100% identical to the amino acid sequence of SEQ ID NO: 294, and a CDR H3 having an amino acid sequence 100% identical to the amino acid sequence of SEQ ID NO: 295; and a VL comprising therein a complementarity determining region (CDR) L1 having an amino acid sequence 100% identical to the amino acid sequence of SEQ ID NO: 297, a CDR L2 having an amino acid sequence 100% identical to the amino acid sequence of KAS, and a CDR L3 having an amino acid sequence 100% identical to the amino acid sequence of SEQ ID NO: 298.

313. The monospecific antibody of any one of claims 1-11 and 307-312, wherein the antibody comprises: (a) a variable heavy chain (VH) region having an amino acid sequence with at least 80% identity to SEQ ID NO: 292 and comprising therein a complementarity determining region (CDR) H1 having an amino acid sequence 100% identical to the amino acid sequence of SEQ ID NO: 293, a CDR H2 having an amino acid sequence 100% identical to the amino acid sequence of SEQ ID NO: 294, and a CDR H3 having an amino acid sequence 100% identical to the amino acid sequence of SEQ ID NO: 295; a variable light chain (VL) region having an amino acid sequence with at least 80% identity to SEQ ID NO: 296 and comprising therein a complementarity determining region (CDR) L1 having an amino acid sequence 100% identical to the amino acid sequence of SEQ ID NO: 297, a CDR L2 having an amino acid sequence 100% identical to the amino acid sequence of KAS, and a CDR L3 having an amino acid sequence 100% identical to the amino acid sequence of SEQ ID NO: 298.

314. The monospecific antibody of any one of claims 1-11, wherein the antibody comprises a variable heavy chain (VH) region having an amino acid sequence with at least 80% identity to SEQ ID NO: 299 and a variable light chain (VL) region having an amino acid sequence with at least 80% identity to SEQ ID NO: 303.

315. The monospecific antibody of any one of claims 1-11 and 314, wherein the antibody comprises a variable heavy chain (VH) region having an amino acid sequence with at least 85% identity to SEQ ID NO: 299 and a variable light chain (VL) region having an amino acid sequence with at least 85% identity to SEQ ID NO: 303.

316. The monospecific antibody of any one of claims 1-11, 314, and 315, wherein the antibody comprises a variable heavy chain (VH) region having an amino acid sequence with at least 90% identity to SEQ ID NO: 299 and a variable light chain (VL) region having an amino acid sequence with at least 90% identity to SEQ ID NO: 303.

317. The monospecific antibody of any one of claims 1-11 and 314-316, wherein the antibody comprises a variable heavy chain (VH) region having an amino acid sequence with at least 95% identity to SEQ ID NO: 299 and a variable light chain (VL) region having an amino acid sequence with at least 95% identity to SEQ ID NO: 303.

318. The monospecific antibody of any one of claims 1-11 and 314-317, wherein the antibody comprises a variable heavy chain (VH) region having an amino acid sequence 100% identical to the amino acid sequence of SEQ ID NO: 299 and a variable light chain (VL) region having an amino acid sequence 100% identical to the amino acid sequence of SEQ ID NO: 303.

319. The monospecific antibody of any one of claims 1-11 and 314-318, wherein the antibody comprises a VH comprising therein a complementarity determining region (CDR) H1 having an amino acid sequence 100% identical to the amino acid sequence of SEQ ID NO: 300, a CDR H2 having an amino acid sequence 100% identical to the amino acid sequence of SEQ ID NO: 301, and a CDR H3 having an amino acid sequence 100% identical to the amino acid sequence of SEQ ID NO: 302; and a VL comprising therein a complementarity determining region (CDR) L1 having an amino acid sequence 100% identical to the amino acid sequence of SEQ ID NO: 304, a CDR L2 having an amino acid sequence 100% identical to the amino acid sequence of AAS, and a CDR L3 having an amino acid sequence 100% identical to the amino acid sequence of SEQ ID NO: 305.

320. The monospecific antibody of any one of claims 1-11 and 314-319, wherein the antibody comprises: (a) a variable heavy chain (VH) region having an amino acid sequence with at least 80% identity to SEQ ID NO: 299 and comprising therein a complementarity determining region (CDR) H1 having an amino acid sequence 100% identical to the amino acid sequence of SEQ ID NO: 300, a CDR H2 having an amino acid sequence 100% identical to the amino acid sequence of SEQ ID NO: 301, and a CDR H3 having an amino acid sequence 100% identical to the amino acid sequence of SEQ ID NO: 302; a variable light chain (VL) region having an amino acid sequence with at least 80% identity to SEQ ID NO: 303 and comprising therein a complementarity determining region (CDR) L1 having an amino acid sequence 100% identical to the amino acid sequence of SEQ ID NO: 304, a CDR L2 having an amino acid sequence 100% identical to the amino acid sequence of AAS, and a CDR L3 having an amino acid sequence 100% identical to the amino acid sequence of SEQ ID NO: 305.

321. The monospecific antibody of any one of claims 1-11, wherein the antibody comprises a variable heavy chain (VH) region having an amino acid sequence with at least 80% identity to SEQ ID NO: 306 and a variable light chain (VL) region having an amino acid sequence with at least 80% identity to SEQ ID NO: 310.

322. The monospecific antibody of any one of claims 1-11, and 321, wherein the antibody comprises a variable heavy chain (VH) region having an amino acid sequence with at least 85% identity to SEQ ID NO: 306 and a variable light chain (VL) region having an amino acid sequence with at least 85% identity to SEQ ID NO: 310.

323. The monospecific antibody of any one of claims 1-11, 321, and 322, wherein the antibody comprises a variable heavy chain (VH) region having an amino acid sequence with at least 90% identity to SEQ ID NO: 306 and a variable light chain (VL) region having an amino acid sequence with at least 90% identity to SEQ ID NO: 310.

324. The monospecific antibody of any one of claims 1-11 and 321-323, wherein the antibody comprises a variable heavy chain (VH) region having an amino acid sequence with at least 95% identity to SEQ ID NO: 306 and a variable light chain (VL) region having an amino acid sequence with at least 95% identity to SEQ ID NO: 310.

325. The monospecific antibody of any one of claims 1-11 and 321-324, wherein the antibody comprises a variable heavy chain (VH) region having an amino acid sequence 100% identical to the amino acid sequence of SEQ ID NO: 306 and a variable light chain (VL) region having an amino acid sequence 100% identical to the amino acid sequence of SEQ ID NO: 310.

326. The monospecific antibody of any one of claims 1-11 and 321-325, wherein the antibody comprises a VH comprising therein a complementarity determining region (CDR) H1 having an amino acid sequence 100% identical to the amino acid sequence of SEQ ID NO: 307, a CDR H2 having an amino acid sequence 100% identical to the amino acid sequence of SEQ ID NO: 308, and a CDR H3 having an amino acid sequence 100% identical to the amino acid sequence of SEQ ID NO: 309; and a VL comprising therein a complementarity determining region (CDR) L1 having an amino acid sequence 100% identical to the amino acid sequence of SEQ ID NO: 311, a CDR L2 having an amino acid sequence 100% identical to the amino acid sequence of DTS, and a CDR L3 having an amino acid sequence 100% identical to the amino acid sequence of SEQ ID NO: 312.

327. The monospecific antibody of any one of claims 1-11 and 321-326, wherein the antibody comprises: (a) a variable heavy chain (VH) region having an amino acid sequence with at least 80% identity to SEQ ID NO: 306 and comprising therein a complementarity determining region (CDR) H1 having an amino acid sequence 100% identical to the amino acid sequence of SEQ ID NO: 307, a CDR H2 having an amino acid sequence 100% identical to the amino acid sequence of SEQ ID NO: 308, and a CDR H3 having an amino acid sequence 100% identical to the amino acid sequence of SEQ ID NO: 309; a variable light chain (VL) region having an amino acid sequence with at least 80% identity to SEQ ID NO: 310 and comprising therein a complementarity determining region (CDR) L1 having an amino acid sequence 100% identical to the amino acid sequence of SEQ ID NO: 311, a CDR L2 having an amino acid sequence 100% identical to the amino acid sequence of DTS, and a CDR L3 having an amino acid sequence 100% identical to the amino acid sequence of SEQ ID NO: 312.

328. The monospecific antibody of any one of claims 1-11 and 321-327, wherein the antibody comprises a first heavy chain (HC) region comprising an amino acid sequence having at least 80% identity to SEQ ID NO: 461, a second heavy chain region comprising an amino acid sequence having at least 80% identity to SEQ ID NO: 481, and a light chain (LC) region, comprising an amino acid sequence having at least 80% identity to SEQ ID NO: 471.

329. The monospecific antibody of any one of claims 1-11, wherein the antibody comprises a variable heavy chain (VH) region having an amino acid sequence with at least 80% identity to SEQ ID NO: 313 and a variable light chain (VL) region having an amino acid sequence with at least 80% identity to SEQ ID NO: 317.

330. The monospecific antibody of any one of claims 1-11 and 329, wherein the antibody comprises a variable heavy chain (VH) region having an amino acid sequence with at least 85% identity to SEQ ID NO: 313 and a variable light chain (VL) region having an amino acid sequence with at least 85% identity to SEQ ID NO: 317.

331. The monospecific antibody of any one of claims 1-11, 329, and 330, wherein the antibody comprises a variable heavy chain (VH) region having an amino acid sequence with at least 90% identity to SEQ ID NO: 313 and a variable light chain (VL) region having an amino acid sequence with at least 90% identity to SEQ ID NO: 317.

332. The monospecific antibody of any one of claims 1-11 and 329-331, wherein the antibody comprises a variable heavy chain (VH) region having an amino acid sequence with at least 95% identity to SEQ ID NO: 313 and a variable light chain (VL) region having an amino acid sequence with at least 95% identity to SEQ ID NO: 317.

333. The monospecific antibody of any one of claims 1-11 and 329-332, wherein the antibody comprises a variable heavy chain (VH) region having an amino acid sequence 100% identical to the amino acid sequence of SEQ ID NO: 313 and a variable light chain (VL) region having an amino acid sequence 100% identical to the amino acid sequence of SEQ ID NO: 317.

334. The monospecific antibody of any one of claims 1-11 and 329-333, wherein the antibody comprises a VH comprising therein a complementarity determining region (CDR) H1 having an amino acid sequence 100% identical to the amino acid sequence of SEQ ID NO: 314, a CDR H2 having an amino acid sequence 100% identical to the amino acid sequence of SEQ ID NO: 315, and a CDR H3 having an amino acid sequence 100% identical to the amino acid sequence of SEQ ID NO: 316; and a VL comprising therein a complementarity determining region (CDR) L1 having an amino acid sequence 100% identical to the amino acid sequence of SEQ ID NO: 318, a CDR L2 having an amino acid sequence 100% identical to the amino acid sequence of DAS, and a CDR L3 having an amino acid sequence 100% identical to the amino acid sequence of SEQ ID NO: 319.

335. The monospecific antibody of any one of claims 1-11 and 329-334, wherein the antibody comprises: (a) a variable heavy chain (VH) region having an amino acid sequence with at least 80% identity to SEQ ID NO: 313 and comprising therein a complementarity determining region (CDR) H1 having an amino acid sequence 100% identical to the amino acid sequence of SEQ ID NO: 314, a CDR H2 having an amino acid sequence 100% identical to the amino acid sequence of SEQ ID NO: 315, and a CDR H3 having an amino acid sequence 100% identical to the amino acid sequence of SEQ ID NO: 316; a variable light chain (VL) region having an amino acid sequence with at least 80% identity to SEQ ID NO: 317 and comprising therein a complementarity determining region (CDR) L1 having an amino acid sequence 100% identical to the amino acid sequence of SEQ ID NO: 318, a CDR L2 having an amino acid sequence 100% identical to the amino acid sequence of DAS, and a CDR L3 having an amino acid sequence 100% identical to the amino acid sequence of SEQ ID NO: 319.

336. The monospecific antibody of any one of claims 1-11, wherein the antibody comprises a variable heavy chain (VH) region having an amino acid sequence with at least 80% identity to SEQ ID NO: 320 and a variable light chain (VL) region having an amino acid sequence with at least 80% identity to SEQ ID NO: 324.

337. The monospecific antibody of any one of claims 1-11 and 336, wherein the antibody comprises a variable heavy chain (VH) region having an amino acid sequence with at least 85% identity to SEQ ID NO: 320 and a variable light chain (VL) region having an amino acid sequence with at least 85% identity to SEQ ID NO: 324.

338. The monospecific antibody of any one of claims 1-11, 336, and 337, wherein the antibody comprises a variable heavy chain (VH) region having an amino acid sequence with at least 90% identity to SEQ ID NO: 320 and a variable light chain (VL) region having an amino acid sequence with at least 90% identity to SEQ ID NO: 324.

339. The monospecific antibody of any one of claims 1-11 and 336-338, wherein the antibody comprises a variable heavy chain (VH) region having an amino acid sequence with at least 95% identity to SEQ ID NO: 320 and a variable light chain (VL) region having an amino acid sequence with at least 95% identity to SEQ ID NO: 324.

340. The monospecific antibody of any one of claims 1-11 and 336-339, wherein the antibody comprises a variable heavy chain (VH) region having an amino acid sequence 100% identical to the amino acid sequence of SEQ ID NO: 320 and a variable light chain (VL) region having an amino acid sequence 100% identical to the amino acid sequence of SEQ ID NO: 324.

341. The monospecific antibody of any one of claims 1-11 and 336-340, wherein the antibody comprises a VH comprising therein a complementarity determining region (CDR) H1 having an amino acid sequence 100% identical to the amino acid sequence of SEQ ID NO: 321, a CDR H2 having an amino acid sequence 100% identical to the amino acid sequence of SEQ ID NO: 322, and a CDR H3 having an amino acid sequence 100% identical to the amino acid sequence of SEQ ID NO: 323; and a VL comprising therein a complementarity determining region (CDR) L1 having an amino acid sequence 100% identical to the amino acid sequence of SEQ ID NO: 325, a CDR L2 having an amino acid sequence 100% identical to the amino acid sequence of STS, and a CDR L3 having an amino acid sequence 100% identical to the amino acid sequence of SEQ ID NO: 326.

342. The monospecific antibody of any one of claims 1-11 and 336-341, wherein the antibody comprises: (a) a variable heavy chain (VH) region having an amino acid sequence with at least 80% identity to SEQ ID NO: 320 and comprising therein a complementarity determining region (CDR) H1 having an amino acid sequence 100% identical to the amino acid sequence of SEQ ID NO: 321, a CDR H2 having an amino acid sequence 100% identical to the amino acid sequence of SEQ ID NO: 322, and a CDR H3 having an amino acid sequence 100% identical to the amino acid sequence of SEQ ID NO: 323; a variable light chain (VL) region having an amino acid sequence with at least 80% identity to SEQ ID NO: 324 and comprising therein a complementarity determining region (CDR) L1 having an amino acid sequence 100% identical to the amino acid sequence of SEQ ID NO: 325, a CDR L2 having an amino acid sequence 100% identical to the amino acid sequence of STS, and a CDR L3 having an amino acid sequence 100% identical to the amino acid sequence of SEQ ID NO: 326.

343. The monospecific antibody of any one of claims 1-11, wherein the antibody comprises a variable heavy chain (VH) region having an amino acid sequence with at least 80% identity to SEQ ID NO: 327 and a variable light chain (VL) region having an amino acid sequence with at least 80% identity to SEQ ID NO: 331.

344. The monospecific antibody of any one of claims 1-11 and 343, wherein the antibody comprises a variable heavy chain (VH) region having an amino acid sequence with at least 85% identity to SEQ ID NO: 327 and a variable light chain (VL) region having an amino acid sequence with at least 85% identity to SEQ ID NO: 331.

345. The monospecific antibody of any one of claims 1-11, 343, and 344, wherein the antibody comprises a variable heavy chain (VH) region having an amino acid sequence with at least 90% identity to SEQ ID NO: 327 and a variable light chain (VL) region having an amino acid sequence with at least 90% identity to SEQ ID NO: 331.

346. The monospecific antibody of any one of claims 1-11 and 343-345, wherein the antibody comprises a variable heavy chain (VH) region having an amino acid sequence with at least 95% identity to SEQ ID NO: 327 and a variable light chain (VL) region having an amino acid sequence with at least 95% identity to SEQ ID NO: 331.

347. The monospecific antibody of any one of claims 1-11 and 343-346, wherein the antibody comprises a variable heavy chain (VH) region having an amino acid sequence 100% identical to the amino acid sequence of SEQ ID NO: 327 and a variable light chain (VL) region having an amino acid sequence 100% identical to the amino acid sequence of SEQ ID NO: 331.

348. The monospecific antibody of any one of claims 1-11 and 343-347, wherein the antibody comprises a VH comprising therein a complementarity determining region (CDR) H1 having an amino acid sequence 100% identical to the amino acid sequence of SEQ ID NO: 328, a CDR H2 having an amino acid sequence 100% identical to the amino acid sequence of SEQ ID NO: 329, and a CDR H3 having an amino acid sequence 100% identical to the amino acid sequence of SEQ ID NO: 330; and a VL comprising therein a complementarity determining region (CDR) L1 having an amino acid sequence 100% identical to the amino acid sequence of SEQ ID NO: 332, a CDR L2 having an amino acid sequence 100% identical to the amino acid sequence of DAS, and a CDR L3 having an amino acid sequence 100% identical to the amino acid sequence of SEQ ID NO: 333.

349. The monospecific antibody of any one of claims 1-11 and 343-348, wherein the antibody comprises: (a) a variable heavy chain (VH) region having an amino acid sequence with at least 80% identity to SEQ ID NO: 327 and comprising therein a complementarity determining region (CDR) H1 having an amino acid sequence 100% identical to the amino acid sequence of SEQ ID NO: 328, a CDR H2 having an amino acid sequence 100% identical to the amino acid sequence of SEQ ID NO: 329, and a CDR H3 having an amino acid sequence 100% identical to the amino acid sequence of SEQ ID NO: 330; a variable light chain (VL) region having an amino acid sequence with at least 80% identity to SEQ ID NO: 331 and comprising therein a complementarity determining region (CDR) L1 having an amino acid sequence 100% identical to the amino acid sequence of SEQ ID NO: 332, a CDR L2 having an amino acid sequence 100% identical to the amino acid sequence of DAS, and a CDR L3 having an amino acid sequence 100% identical to the amino acid sequence of SEQ ID NO: 333.

350. The monospecific antibody of any one of claims 1-11, wherein the antibody comprises a variable heavy chain (VH) region having an amino acid sequence with at least 80% identity to SEQ ID NO: 334 and a variable light chain (VL) region having an amino acid sequence with at least 80% identity to SEQ ID NO: 338.

351. The monospecific antibody of any one of claims 1-11 and 350, wherein the antibody comprises a variable heavy chain (VH) region having an amino acid sequence with at least 85% identity to SEQ ID NO: 334 and a variable light chain (VL) region having an amino acid sequence with at least 85% identity to SEQ ID NO: 338.

352. The monospecific antibody of any one of claims 1-11, 350, and 351, wherein the antibody comprises a variable heavy chain (VH) region having an amino acid sequence with at least 90% identity to SEQ ID NO: 334 and a variable light chain (VL) region having an amino acid sequence with at least 90% identity to SEQ ID NO: 338.

353. The monospecific antibody of any one of claims 1-11 and 350-352, wherein the antibody comprises a variable heavy chain (VH) region having an amino acid sequence with at least 95% identity to SEQ ID NO: 334 and a variable light chain (VL) region having an amino acid sequence with at least 95% identity to SEQ ID NO: 338.

354. The monospecific antibody of any one of claims 1-11 and 350-353, wherein the antibody comprises a variable heavy chain (VH) region having an amino acid sequence 100% identical to the amino acid sequence of SEQ ID NO: 334 and a variable light chain (VL) region having an amino acid sequence 100% identical to the amino acid sequence of SEQ ID NO: 338.

355. The monospecific antibody of any one of claims 1-11 and 350-354, wherein the antibody comprises a VH comprising therein a complementarity determining region (CDR) H1 having an amino acid sequence 100% identical to the amino acid sequence of SEQ ID NO: 335, a CDR H2 having an amino acid sequence 100% identical to the amino acid sequence of SEQ ID NO: 336, and a CDR H3 having an amino acid sequence 100% identical to the amino acid sequence of SEQ ID NO: 337; and a VL comprising therein a complementarity determining region (CDR) L1 having an amino acid sequence 100% identical to the amino acid sequence of SEQ ID NO: 339, a CDR L2 having an amino acid sequence 100% identical to the amino acid sequence of GAS, and a CDR L3 having an amino acid sequence 100% identical to the amino acid sequence of SEQ ID NO: 340.

356. The monospecific antibody of any one of claims 1-11 and 350-355, wherein the antibody comprises: (a) a variable heavy chain (VH) region having an amino acid sequence with at least 80% identity to SEQ ID NO: 334 and comprising therein a complementarity determining region (CDR) H1 having an amino acid sequence 100% identical to the amino acid sequence of SEQ ID NO: 335, a CDR H2 having an amino acid sequence 100% identical to the amino acid sequence of SEQ ID NO: 336, and a CDR H3 having an amino acid sequence 100% identical to the amino acid sequence of SEQ ID NO: 337; a variable light chain (VL) region having an amino acid sequence with at least 80% identity to SEQ ID NO: 338 and comprising therein a complementarity determining region (CDR) L1 having an amino acid sequence 100% identical to the amino acid sequence of SEQ ID NO: 339, a CDR L2 having an amino acid sequence 100% identical to the amino acid sequence of GAS, and a CDR L3 having an amino acid sequence 100% identical to the amino acid sequence of SEQ ID NO: 340.

357. The monospecific antibody of any one of claims 1-11, wherein the antibody comprises a variable heavy chain (VH) region having an amino acid sequence with at least 80% identity to SEQ ID NO: 341 and a variable light chain (VL) region having an amino acid sequence with at least 80% identity to SEQ ID NO: 345.

358. The monospecific antibody of any one of claims 1-11 and 357, wherein the antibody comprises a variable heavy chain (VH) region having an amino acid sequence with at least 85% identity to SEQ ID NO: 341 and a variable light chain (VL) region having an amino acid sequence with at least 85% identity to SEQ ID NO: 345.

359. The monospecific antibody of any one of claims 1-11, 357, and 358, wherein the antibody comprises a variable heavy chain (VH) region having an amino acid sequence with at least 90% identity to SEQ ID NO: 341 and a variable light chain (VL) region having an amino acid sequence with at least 90% identity to SEQ ID NO: 345.

360. The monospecific antibody of any one of claims 1-11 and 357-359, wherein the antibody comprises a variable heavy chain (VH) region having an amino acid sequence with at least 95% identity to SEQ ID NO: 341 and a variable light chain (VL) region having an amino acid sequence with at least 95% identity to SEQ ID NO: 345.

361. The monospecific antibody of any one of claims 1-11 and 357-360, wherein the antibody comprises a variable heavy chain (VH) region having an amino acid sequence 100% identical to the amino acid sequence of SEQ ID NO: 341 and a variable light chain (VL) region having an amino acid sequence 100% identical to the amino acid sequence of SEQ ID NO: 345.

362. The monospecific antibody of any one of claims 1-11 and 357-361, wherein the antibody comprises a VH comprising therein a complementarity determining region (CDR) H1 having an amino acid sequence 100% identical to the amino acid sequence of SEQ ID NO: 342, a CDR H2 having an amino acid sequence 100% identical to the amino acid sequence of SEQ ID NO: 343, and a CDR H3 having an amino acid sequence 100% identical to the amino acid sequence of SEQ ID NO: 344; and a VL comprising therein a complementarity determining region (CDR) L1 having an amino acid sequence 100% identical to the amino acid sequence of SEQ ID NO: 346, a CDR L2 having an amino acid sequence 100% identical to the amino acid sequence of DAS, and a CDR L3 having an amino acid sequence 100% identical to the amino acid sequence of SEQ ID NO: 347.

363. The monospecific antibody of any one of claims 1-11 and 357-362, wherein the antibody comprises: (a) a variable heavy chain (VH) region having an amino acid sequence with at least 80% identity to SEQ ID NO: 341 and comprising therein a complementarity determining region (CDR) H1 having an amino acid sequence 100% identical to the amino acid sequence of SEQ ID NO: 342, a CDR H2 having an amino acid sequence 100% identical to the amino acid sequence of SEQ ID NO: 343, and a CDR H3 having an amino acid sequence 100% identical to the amino acid sequence of SEQ ID NO: 344; a variable light chain (VL) region having an amino acid sequence with at least 80% identity to SEQ ID NO: 345 and comprising therein a complementarity determining region (CDR) L1 having an amino acid sequence 100% identical to the amino acid sequence of SEQ ID NO: 346, a CDR L2 having an amino acid sequence 100% identical to the amino acid sequence of DAS, and a CDR L3 having an amino acid sequence 100% identical to the amino acid sequence of SEQ ID NO: 347.

364. The monospecific antibody of any one of claims 1-11, wherein the antibody comprises a variable heavy chain (VH) region having an amino acid sequence with at least 80% identity to SEQ ID NO: 348 and a variable light chain (VL) region having an amino acid sequence with at least 80% identity to SEQ ID NO: 352.

365. The monospecific antibody of any one of claims 1-11 and 364, wherein the antibody comprises a variable heavy chain (VH) region having an amino acid sequence with at least 85% identity to SEQ ID NO: 348 and a variable light chain (VL) region having an amino acid sequence with at least 85% identity to SEQ ID NO: 352.

366. The monospecific antibody of any one of claims 1-11, 364, and 365, wherein the antibody comprises a variable heavy chain (VH) region having an amino acid sequence with at least 90% identity to SEQ ID NO: 348 and a variable light chain (VL) region having an amino acid sequence with at least 90% identity to SEQ ID NO: 352.

367. The monospecific antibody of any one of claims 1-11 and 364-366, wherein the antibody comprises a variable heavy chain (VH) region having an amino acid sequence with at least 95% identity to SEQ ID NO: 348 and a variable light chain (VL) region having an amino acid sequence with at least 95% identity to SEQ ID NO: 352.

368. The monospecific antibody of any one of claims 1-11 and 364-367, wherein the antibody comprises a variable heavy chain (VH) region having an amino acid sequence 100% identical to the amino acid sequence of SEQ ID NO: 348 and a variable light chain (VL) region having an amino acid sequence 100% identical to the amino acid sequence of SEQ ID NO: 352.

369. The monospecific antibody of any one of claims 1-11 and 364-368, wherein the antibody comprises a VH comprising therein a complementarity determining region (CDR) H1 having an amino acid sequence 100% identical to the amino acid sequence of SEQ ID NO: 349, a CDR H2 having an amino acid sequence 100% identical to the amino acid sequence of SEQ ID NO: 350, and a CDR H3 having an amino acid sequence 100% identical to the amino acid sequence of SEQ ID NO: 351; and a VL comprising therein a complementarity determining region (CDR) L1 having an amino acid sequence 100% identical to the amino acid sequence of SEQ ID NO: 353, a CDR L2 having an amino acid sequence 100% identical to the amino acid sequence of DTS, and a CDR L3 having an amino acid sequence 100% identical to the amino acid sequence of SEQ ID NO: 354.

370. The monospecific antibody of any one of claims 1-11 and 364-369, wherein the antibody comprises: (a) a variable heavy chain (VH) region having an amino acid sequence with at least 80% identity to SEQ ID NO: 348 and comprising therein a complementarity determining region (CDR) H1 having an amino acid sequence 100% identical to the amino acid sequence of SEQ ID NO: 349, a CDR H2 having an amino acid sequence 100% identical to the amino acid sequence of SEQ ID NO: 350, and a CDR H3 having an amino acid sequence 100% identical to the amino acid sequence of SEQ ID NO: 351; a variable light chain (VL) region having an amino acid sequence with at least 80% identity to SEQ ID NO: 352 and comprising therein a complementarity determining region (CDR) L1 having an amino acid sequence 100% identical to the amino acid sequence of SEQ ID NO: 353, a CDR L2 having an amino acid sequence 100% identical to the amino acid sequence of DTS, and a CDR L3 having an amino acid sequence 100% identical to the amino acid sequence of SEQ ID NO: 354.

371. The monospecific antibody of any one of claims 1-11, wherein the antibody comprises a variable heavy chain (VH) region having an amino acid sequence with at least 80% identity to SEQ ID NO: 355 and a variable light chain (VL) region having an amino acid sequence with at least 80% identity to SEQ ID NO: 359.

372. The monospecific antibody of any one of claims 1-11 and 371, wherein the antibody comprises a variable heavy chain (VH) region having an amino acid sequence with at least 85% identity to SEQ ID NO: 355 and a variable light chain (VL) region having an amino acid sequence with at least 85% identity to SEQ ID NO: 359.

373. The monospecific antibody of any one of claims 1-11, 371, and 372, wherein the antibody comprises a variable heavy chain (VH) region having an amino acid sequence with at least 90% identity to SEQ ID NO: 355 and a variable light chain (VL) region having an amino acid sequence with at least 90% identity to SEQ ID NO: 359.

374. The monospecific antibody of any one of claims 1-11 and 371-373, wherein the antibody comprises a variable heavy chain (VH) region having an amino acid sequence with at least 95% identity to SEQ ID NO: 355 and a variable light chain (VL) region having an amino acid sequence with at least 95% identity to SEQ ID NO: 359.

375. The monospecific antibody of any one of claims 1-11 and 371-374, wherein the antibody comprises a variable heavy chain (VH) region having an amino acid sequence 100% identical to the amino acid sequence of SEQ ID NO: 355 and a variable light chain (VL) region having an amino acid sequence 100% identical to the amino acid sequence of SEQ ID NO: 359.

376. The monospecific antibody of any one of claims 1-11 and 371-375, wherein the antibody comprises a VH comprising therein a complementarity determining region (CDR) H1 having an amino acid sequence 100% identical to the amino acid sequence of SEQ ID NO: 356, a CDR H2 having an amino acid sequence 100% identical to the amino acid sequence of SEQ ID NO: 357, and a CDR H3 having an amino acid sequence 100% identical to the amino acid sequence of SEQ ID NO: 358; and a VL comprising therein a complementarity determining region (CDR) L1 having an amino acid sequence 100% identical to the amino acid sequence of SEQ ID NO: 360, a CDR L2 having an amino acid sequence 100% identical to the amino acid sequence of AAS, and a CDR L3 having an amino acid sequence 100% identical to the amino acid sequence of SEQ ID NO: 361.

377. The monospecific antibody of any one of claims 1-11 and 371-378, wherein the antibody comprises: (a) a variable heavy chain (VH) region having an amino acid sequence with at least 80% identity to SEQ ID NO: 355 and comprising therein a complementarity determining region (CDR) H1 having an amino acid sequence 100% identical to the amino acid sequence of SEQ ID NO: 356, a CDR H2 having an amino acid sequence 100% identical to the amino acid sequence of SEQ ID NO: 357, and a CDR H3 having an amino acid sequence 100% identical to the amino acid sequence of SEQ ID NO: 358; a variable light chain (VL) region having an amino acid sequence with at least 80% identity to SEQ ID NO: 359 and comprising therein a complementarity determining region (CDR) L1 having an amino acid sequence 100% identical to the amino acid sequence of SEQ ID NO: 360, a CDR L2 having an amino acid sequence 100% identical to the amino acid sequence of AAS, and a CDR L3 having an amino acid sequence 100% identical to the amino acid sequence of SEQ ID NO: 361.

378. The monospecific antibody of any one of claims 1-11, wherein the antibody comprises a variable heavy chain (VH) region having an amino acid sequence with at least 80% identity to SEQ ID NO: 362 and a variable light chain (VL) region having an amino acid sequence with at least 80% identity to SEQ ID NO: 366.

379. The monospecific antibody of any one of claims 1-11 and 378, wherein the antibody comprises a variable heavy chain (VH) region having an amino acid sequence with at least 85% identity to SEQ ID NO: 362 and a variable light chain (VL) region having an amino acid sequence with at least 85% identity to SEQ ID NO: 366.

380. The monospecific antibody of any one of claims 1-11, 378, and 379, wherein the antibody comprises a variable heavy chain (VH) region having an amino acid sequence with at least 90% identity to SEQ ID NO: 362 and a variable light chain (VL) region having an amino acid sequence with at least 90% identity to SEQ ID NO: 366.

381. The monospecific antibody of any one of claims 1-11 and 378-380, wherein the antibody comprises a variable heavy chain (VH) region having an amino acid sequence with at least 95% identity to SEQ ID NO: 362 and a variable light chain (VL) region having an amino acid sequence with at least 95% identity to SEQ ID NO: 366.

382. The monospecific antibody of any one of claims 1-11 and 378-381, wherein the antibody comprises a variable heavy chain (VH) region having an amino acid sequence 100% identical to the amino acid sequence of SEQ ID NO: 362 and a variable light chain (VL) region having an amino acid sequence 100% identical to the amino acid sequence of SEQ ID NO: 366.

383. The monospecific antibody of any one of claims 1-11 and 378-382, wherein the antibody comprises a VH comprising therein a complementarity determining region (CDR) H1 having an amino acid sequence 100% identical to the amino acid sequence of SEQ ID NO: 363, a CDR H2 having an amino acid sequence 100% identical to the amino acid sequence of SEQ ID NO: 364, and a CDR H3 having an amino acid sequence 100% identical to the amino acid sequence of SEQ ID NO: 365; and a VL comprising therein a complementarity determining region (CDR) L1 having an amino acid sequence 100% identical to the amino acid sequence of SEQ ID NO: 367, a CDR L2 having an amino acid sequence 100% identical to the amino acid sequence of AAS, and a CDR L3 having an amino acid sequence 100% identical to the amino acid sequence of SEQ ID NO: 368.

384. The monospecific antibody of any one of claims 1-11 and 378-383, wherein the antibody comprises: (a) a variable heavy chain (VH) region having an amino acid sequence with at least 80% identity to SEQ ID NO: 362 and comprising therein a complementarity determining region (CDR) H1 having an amino acid sequence 100% identical to the amino acid sequence of SEQ ID NO: 363, a CDR H2 having an amino acid sequence 100% identical to the amino acid sequence of SEQ ID NO: 364, and a CDR H3 having an amino acid sequence 100% identical to the amino acid sequence of SEQ ID NO: 365; a variable light chain (VL) region having an amino acid sequence with at least 80% identity to SEQ ID NO: 366 and comprising therein a complementarity determining region (CDR) L1 having an amino acid sequence 100% identical to the amino acid sequence of SEQ ID NO: 367, a CDR L2 having an amino acid sequence 100% identical to the amino acid sequence of AAS, and a CDR L3 having an amino acid sequence 100% identical to the amino acid sequence of SEQ ID NO: 368.

385. The monospecific antibody of any one of claims 1-11, wherein the antibody comprises a variable heavy chain (VH) region having an amino acid sequence with at least 80% identity to SEQ ID NO: 369 and a variable light chain (VL) region having an amino acid sequence with at least 80% identity to SEQ ID NO: 373.

386. The monospecific antibody of any one of claims 1-11 and 385, wherein the antibody comprises a variable heavy chain (VH) region having an amino acid sequence with at least 85% identity to SEQ ID NO: 369 and a variable light chain (VL) region having an amino acid sequence with at least 85% identity to SEQ ID NO: 373.

387. The monospecific antibody of any one of claims 1-11, 385, and 386, wherein the antibody comprises a variable heavy chain (VH) region having an amino acid sequence with at least 90% identity to SEQ ID NO: 369 and a variable light chain (VL) region having an amino acid sequence with at least 90% identity to SEQ ID NO: 373.

388. The monospecific antibody of any one of claims 1-11 and 385-387, wherein the antibody comprises a variable heavy chain (VH) region having an amino acid sequence with at least 95% identity to SEQ ID NO: 369 and a variable light chain (VL) region having an amino acid sequence with at least 95% identity to SEQ ID NO: 373.

389. The monospecific antibody of any one of claims 1-11 and 385-388, wherein the antibody comprises a variable heavy chain (VH) region having an amino acid sequence 100% identical to the amino acid sequence of SEQ ID NO: 369 and a variable light chain (VL) region having an amino acid sequence 100% identical to the amino acid sequence of SEQ ID NO: 373.

390. The monospecific antibody of any one of claims 1-11 and 385-389, wherein the antibody comprises a VH comprising therein a complementarity determining region (CDR) H1 having an amino acid sequence 100% identical to the amino acid sequence of SEQ ID NO: 370, a CDR H2 having an amino acid sequence 100% identical to the amino acid sequence of SEQ ID NO: 371, and a CDR H3 having an amino acid sequence 100% identical to the amino acid sequence of SEQ ID NO: 372; and a VL comprising therein a complementarity determining region (CDR) L1 having an amino acid sequence 100% identical to the amino acid sequence of SEQ ID NO: 374, a CDR L2 having an amino acid sequence 100% identical to the amino acid sequence of GTS, and a CDR L3 having an amino acid sequence 100% identical to the amino acid sequence of SEQ ID NO: 375.

391. The monospecific antibody of any one of claims 1-11 and 385-380, wherein the antibody comprises: (a) a variable heavy chain (VH) region having an amino acid sequence with at least 80% identity to SEQ ID NO: 369 and comprising therein a complementarity determining region (CDR) H1 having an amino acid sequence 100% identical to the amino acid sequence of SEQ ID NO: 370, a CDR H2 having an amino acid sequence 100% identical to the amino acid sequence of SEQ ID NO: 371, and a CDR H3 having an amino acid sequence 100% identical to the amino acid sequence of SEQ ID NO: 372; a variable light chain (VL) region having an amino acid sequence with at least 80% identity to SEQ ID NO: 373 and comprising therein a complementarity determining region (CDR) L1 having an amino acid sequence 100% identical to the amino acid sequence of SEQ ID NO: 374, a CDR L2 having an amino acid sequence 100% identical to the amino acid sequence of GTS, and a CDR L3 having an amino acid sequence 100% identical to the amino acid sequence of SEQ ID NO: 375.

392. The monospecific antibody of any one of claims 1-11, wherein the antibody comprises a variable heavy chain (VH) region having an amino acid sequence with at least 80% identity to SEQ ID NO: 376 and a variable light chain (VL) region having an amino acid sequence with at least 80% identity to SEQ ID NO: 380.

393. The monospecific antibody of any one of claims 1-11 and 392, wherein the antibody comprises a variable heavy chain (VH) region having an amino acid sequence with at least 85% identity to SEQ ID NO: 376 and a variable light chain (VL) region having an amino acid sequence with at least 85% identity to SEQ ID NO: 380.

394. The monospecific antibody of any one of claims 1-11, 392, and 393, wherein the antibody comprises a variable heavy chain (VH) region having an amino acid sequence with at least 90% identity to SEQ ID NO: 376 and a variable light chain (VL) region having an amino acid sequence with at least 90% identity to SEQ ID NO: 380.

395. The monospecific antibody of any one of claims 1-11 and 392-394, wherein the antibody comprises a variable heavy chain (VH) region having an amino acid sequence with at least 95% identity to SEQ ID NO: 376 and a variable light chain (VL) region having an amino acid sequence with at least 95% identity to SEQ ID NO: 380.

396. The monospecific antibody of any one of claims 1-11 and 392-395, wherein the antibody comprises a variable heavy chain (VH) region having an amino acid sequence 100% identical to the amino acid sequence of SEQ ID NO: 376 and a variable light chain (VL) region having an amino acid sequence 100% identical to the amino acid sequence of SEQ ID NO: 380.

397. The monospecific antibody of any one of claims 1-11 and 392-396, wherein the antibody comprises a VH comprising therein a complementarity determining region (CDR) H1 having an amino acid sequence 100% identical to the amino acid sequence of SEQ ID NO: 377, a CDR H2 having an amino acid sequence 100% identical to the amino acid sequence of SEQ ID NO: 378, and a CDR H3 having an amino acid sequence 100% identical to the amino acid sequence of SEQ ID NO: 379; and a VL comprising therein a complementarity determining region (CDR) L1 having an amino acid sequence 100% identical to the amino acid sequence of SEQ ID NO: 381, a CDR L2 having an amino acid sequence 100% identical to the amino acid sequence of ETS, and a CDR L3 having an amino acid sequence 100% identical to the amino acid sequence of SEQ ID NO: 382.

398. The monospecific antibody of any one of claims 1-11 and 392-397, wherein the antibody comprises: (a) a variable heavy chain (VH) region having an amino acid sequence with at least 80% identity to SEQ ID NO: 376 and comprising therein a complementarity determining region (CDR) H1 having an amino acid sequence 100% identical to the amino acid sequence of SEQ ID NO: 377, a CDR H2 having an amino acid sequence 100% identical to the amino acid sequence of SEQ ID NO: 378, and a CDR H3 having an amino acid sequence 100% identical to the amino acid sequence of SEQ ID NO: 379; a variable light chain (VL) region having an amino acid sequence with at least 80% identity to SEQ ID NO: 380 and comprising therein a complementarity determining region (CDR) L1 having an amino acid sequence 100% identical to the amino acid sequence of SEQ ID NO: 381, a CDR L2 having an amino acid sequence 100% identical to the amino acid sequence of ETS, and a CDR L3 having an amino acid sequence 100% identical to the amino acid sequence of SEQ ID NO: 382.

399. The monospecific antibody of any one of claims 1-11, wherein the antibody comprises a variable heavy chain (VH) region having an amino acid sequence with at least 80% identity to SEQ ID NO: 383 and a variable light chain (VL) region having an amino acid sequence with at least 80% identity to SEQ ID NO: 387.

400. The monospecific antibody of any one of claims 1-11 and 399, wherein the antibody comprises a variable heavy chain (VH) region having an amino acid sequence with at least 85% identity to SEQ ID NO: 383 and a variable light chain (VL) region having an amino acid sequence with at least 85% identity to SEQ ID NO: 387.

401. The monospecific antibody of any one of claims 1-11, 399, and 400, wherein the antibody comprises a variable heavy chain (VH) region having an amino acid sequence with at least 90% identity to SEQ ID NO: 383 and a variable light chain (VL) region having an amino acid sequence with at least 90% identity to SEQ ID NO: 387.

402. The monospecific antibody of any one of claims 1-11 and 399-401, wherein the antibody comprises a variable heavy chain (VH) region having an amino acid sequence with at least 95% identity to SEQ ID NO: 383 and a variable light chain (VL) region having an amino acid sequence with at least 95% identity to SEQ ID NO: 387.

403. The monospecific antibody of any one of claims 1-11 and 399-402, wherein the antibody comprises a variable heavy chain (VH) region having an amino acid sequence 100% identical to the amino acid sequence of SEQ ID NO: 383 and a variable light chain (VL) region having an amino acid sequence 100% identical to the amino acid sequence of SEQ ID NO: 387.

404. The monospecific antibody of any one of claims 1-11 and 399-403, wherein the antibody comprises a VH comprising therein a complementarity determining region (CDR) H1 having an amino acid sequence 100% identical to the amino acid sequence of SEQ ID NO: 384, a CDR H2 having an amino acid sequence 100% identical to the amino acid sequence of SEQ ID NO: 385, and a CDR H3 having an amino acid sequence 100% identical to the amino acid sequence of SEQ ID NO: 386; and a VL comprising therein a complementarity determining region (CDR) L1 having an amino acid sequence 100% identical to the amino acid sequence of SEQ ID NO: 388, a CDR L2 having an amino acid sequence 100% identical to the amino acid sequence of GAS, and a CDR L3 having an amino acid sequence 100% identical to the amino acid sequence of SEQ ID NO: 389.

405. The monospecific antibody of any one of claims 1-11 and 399-404, wherein the antibody comprises: (a) a variable heavy chain (VH) region having an amino acid sequence with at least 80% identity to SEQ ID NO: 383 and comprising therein a complementarity determining region (CDR) H1 having an amino acid sequence 100% identical to the amino acid sequence of SEQ ID NO: 384, a CDR H2 having an amino acid sequence 100% identical to the amino acid sequence of SEQ ID NO: 385, and a CDR H3 having an amino acid sequence 100% identical to the amino acid sequence of SEQ ID NO: 386; a variable light chain (VL) region having an amino acid sequence with at least 80% identity to SEQ ID NO: 387 and comprising therein a complementarity determining region (CDR) L1 having an amino acid sequence 100% identical to the amino acid sequence of SEQ ID NO: 388, a CDR L2 having an amino acid sequence 100% identical to the amino acid sequence of GAS, and a CDR L3 having an amino acid sequence 100% identical to the amino acid sequence of SEQ ID NO: 389.

406. The monospecific antibody of any one of claims 1-11, wherein the antibody comprises a variable heavy chain (VH) region having an amino acid sequence with at least 80% identity to SEQ ID NO: 390 and a variable light chain (VL) region having an amino acid sequence with at least 80% identity to SEQ ID NO: 394.

407. The monospecific antibody of any one of claims 1-11 and 406, wherein the antibody comprises a variable heavy chain (VH) region having an amino acid sequence with at least 85% identity to SEQ ID NO: 390 and a variable light chain (VL) region having an amino acid sequence with at least 85% identity to SEQ ID NO: 394.

408. The monospecific antibody of any one of claims 1-11, 406, and 407, wherein the antibody comprises a variable heavy chain (VH) region having an amino acid sequence with at least 90% identity to SEQ ID NO: 390 and a variable light chain (VL) region having an amino acid sequence with at least 90% identity to SEQ ID NO: 394.

409. The monospecific antibody of any one of claims 1-11 and 406-408, wherein the antibody comprises a variable heavy chain (VH) region having an amino acid sequence with at least 95% identity to SEQ ID NO: 390 and a variable light chain (VL) region having an amino acid sequence with at least 95% identity to SEQ ID NO: 394.

410. The monospecific antibody of any one of claims 1-11 and 406-409, wherein the antibody comprises a variable heavy chain (VH) region having an amino acid sequence 100% identical to the amino acid sequence of SEQ ID NO: 390 and a variable light chain (VL) region having an amino acid sequence 100% identical to the amino acid sequence of SEQ ID NO: 394.

411. The monospecific antibody of any one of claims 1-11 and 406-410, wherein the antibody comprises a VH comprising therein a complementarity determining region (CDR) H1 having an amino acid sequence 100% identical to the amino acid sequence of SEQ ID NO: 391, a CDR H2 having an amino acid sequence 100% identical to the amino acid sequence of SEQ ID NO: 392, and a CDR H3 having an amino acid sequence 100% identical to the amino acid sequence of SEQ ID NO: 393; and a VL comprising therein a complementarity determining region (CDR) L1 having an amino acid sequence 100% identical to the amino acid sequence of SEQ ID NO: 395, a CDR L2 having an amino acid sequence 100% identical to the amino acid sequence of DAS, and a CDR L3 having an amino acid sequence 100% identical to the amino acid sequence of SEQ ID NO: 396.

412. The monospecific antibody of any one of claims 1-11 and 406-411, wherein the antibody comprises: (a) a variable heavy chain (VH) region having an amino acid sequence with at least 80% identity to SEQ ID NO: 390 and comprising therein a complementarity determining region (CDR) H1 having an amino acid sequence 100% identical to the amino acid sequence of SEQ ID NO: 391, a CDR H2 having an amino acid sequence 100% identical to the amino acid sequence of SEQ ID NO: 392, and a CDR H3 having an amino acid sequence 100% identical to the amino acid sequence of SEQ ID NO: 393; a variable light chain (VL) region having an amino acid sequence with at least 80% identity to SEQ ID NO: 394 and comprising therein a complementarity determining region (CDR) L1 having an amino acid sequence 100% identical to the amino acid sequence of SEQ ID NO: 395, a CDR L2 having an amino acid sequence 100% identical to the amino acid sequence of DAS, and a CDR L3 having an amino acid sequence 100% identical to the amino acid sequence of SEQ ID NO: 396.

413. The monospecific antibody of any one of claims 1-11 and 406-412, wherein the antibody comprises a first heavy chain (HC) region comprising an amino acid sequence having at least 80% identity to SEQ ID NO: 462, a second heavy chain region comprising an amino acid sequence having at least 80% identity to SEQ ID NO: 482, and a light chain (LC) region, comprising an amino acid sequence having at least 80% identity to SEQ ID NO: 472.

414. The monospecific antibody of any one of claims 1-11, wherein the antibody comprises a variable heavy chain (VH) region having an amino acid sequence with at least 80% identity to SEQ ID NO: 397 and a variable light chain (VL) region having an amino acid sequence with at least 80% identity to SEQ ID NO: 401.

415. The monospecific antibody of any one of claims 1-11 and 414, wherein the antibody comprises a variable heavy chain (VH) region having an amino acid sequence with at least 85% identity to SEQ ID NO: 397 and a variable light chain (VL) region having an amino acid sequence with at least 85% identity to SEQ ID NO: 401.

416. The monospecific antibody of any one of claims 1-11, 414, and 415, wherein the antibody comprises a variable heavy chain (VH) region having an amino acid sequence with at least 90% identity to SEQ ID NO: 397 and a variable light chain (VL) region having an amino acid sequence with at least 90% identity to SEQ ID NO: 401.

417. The monospecific antibody of any one of claims 1-11 and 414-416, wherein the antibody comprises a variable heavy chain (VH) region having an amino acid sequence with at least 95% identity to SEQ ID NO: 397 and a variable light chain (VL) region having an amino acid sequence with at least 95% identity to SEQ ID NO: 401.

418. The monospecific antibody of any one of claims 1-11 and 414-417, wherein the antibody comprises a variable heavy chain (VH) region having an amino acid sequence 100% identical to the amino acid sequence of SEQ ID NO: 397 and a variable light chain (VL) region having an amino acid sequence 100% identical to the amino acid sequence of SEQ ID NO: 401.

419. The monospecific antibody of any one of claims 1-11 and 414-418, wherein the antibody comprises a VH comprising therein a complementarity determining region (CDR) H1 having an amino acid sequence 100% identical to the amino acid sequence of SEQ ID NO: 398, a CDR H2 having an amino acid sequence 100% identical to the amino acid sequence of SEQ ID NO: 399, and a CDR H3 having an amino acid sequence 100% identical to the amino acid sequence of SEQ ID NO: 400; and a VL comprising therein a complementarity determining region (CDR) L1 having an amino acid sequence 100% identical to the amino acid sequence of SEQ ID NO: 402, a CDR L2 having an amino acid sequence 100% identical to the amino acid sequence of GAS, and a CDR L3 having an amino acid sequence 100% identical to the amino acid sequence of SEQ ID NO: 403.

420. The monospecific antibody of any one of claims 1-11 and 414-419, wherein the antibody comprises: (a) a variable heavy chain (VH) region having an amino acid sequence with at least 80% identity to SEQ ID NO: 397 and comprising therein a complementarity determining region (CDR) H1 having an amino acid sequence 100% identical to the amino acid sequence of SEQ ID NO: 398, a CDR H2 having an amino acid sequence 100% identical to the amino acid sequence of SEQ ID NO: 399, and a CDR H3 having an amino acid sequence 100% identical to the amino acid sequence of SEQ ID NO: 400; a variable light chain (VL) region having an amino acid sequence with at least 80% identity to SEQ ID NO: 401 and comprising therein a complementarity determining region (CDR) L1 having an amino acid sequence 100% identical to the amino acid sequence of SEQ ID NO: 402, a CDR L2 having an amino acid sequence 100% identical to the amino acid sequence of GAS, and a CDR L3 having an amino acid sequence 100% identical to the amino acid sequence of SEQ ID NO: 403.

421. The monospecific antibody of any one of claims 1-11, wherein the antibody comprises a variable heavy chain (VH) region having an amino acid sequence with at least 80% identity to SEQ ID NO: 404 and a variable light chain (VL) region having an amino acid sequence with at least 80% identity to SEQ ID NO: 408.

422. The monospecific antibody of any one of claims 1-11 and 421, wherein the antibody comprises a variable heavy chain (VH) region having an amino acid sequence with at least 85% identity to SEQ ID NO: 404 and a variable light chain (VL) region having an amino acid sequence with at least 85% identity to SEQ ID NO: 408.

423. The monospecific antibody of any one of claims 1-11, 421, and 422, wherein the antibody comprises a variable heavy chain (VH) region having an amino acid sequence with at least 90% identity to SEQ ID NO: 404 and a variable light chain (VL) region having an amino acid sequence with at least 90% identity to SEQ ID NO: 408.

424. The monospecific antibody of any one of claims 1-11 and 421-423, wherein the antibody comprises a variable heavy chain (VH) region having an amino acid sequence with at least 95% identity to SEQ ID NO: 404 and a variable light chain (VL) region having an amino acid sequence with at least 95% identity to SEQ ID NO: 408.

425. The monospecific antibody of any one of claims 1-11 and 421-424, wherein the antibody comprises a variable heavy chain (VH) region having an amino acid sequence 100% identical to the amino acid sequence of SEQ ID NO: 404 and a variable light chain (VL) region having an amino acid sequence 100% identical to the amino acid sequence of SEQ ID NO: 408.

426. The monospecific antibody of any one of claims 1-11 and 421-425, wherein the antibody comprises a VH comprising therein a complementarity determining region (CDR) H1 having an amino acid sequence 100% identical to the amino acid sequence of SEQ ID NO: 405, a CDR H2 having an amino acid sequence 100% identical to the amino acid sequence of SEQ ID NO: 406, and a CDR H3 having an amino acid sequence 100% identical to the amino acid sequence of SEQ ID NO: 407; and a VL comprising therein a complementarity determining region (CDR) L1 having an amino acid sequence 100% identical to the amino acid sequence of SEQ ID NO: 409, a CDR L2 having an amino acid sequence 100% identical to the amino acid sequence of DAS, and a CDR L3 having an amino acid sequence 100% identical to the amino acid sequence of SEQ ID NO: 410.

427. The monospecific antibody of any one of claims 1-11 and 421-426, wherein the antibody comprises: (a) a variable heavy chain (VH) region having an amino acid sequence with at least 80% identity to SEQ ID NO: 404 and comprising therein a complementarity determining region (CDR) H1 having an amino acid sequence 100% identical to the amino acid sequence of SEQ ID NO: 405, a CDR H2 having an amino acid sequence 100% identical to the amino acid sequence of SEQ ID NO: 406, and a CDR H3 having an amino acid sequence 100% identical to the amino acid sequence of SEQ ID NO: 407; a variable light chain (VL) region having an amino acid sequence with at least 80% identity to SEQ ID NO: 408 and comprising therein a complementarity determining region (CDR) L1 having an amino acid sequence 100% identical to the amino acid sequence of SEQ ID NO: 409, a CDR L2 having an amino acid sequence 100% identical to the amino acid sequence of DAS, and a CDR L3 having an amino acid sequence 100% identical to the amino acid sequence of SEQ ID NO: 410.

428. The monospecific antibody of any one of claims 1-11, wherein the antibody comprises a variable heavy chain (VH) region having an amino acid sequence with at least 80% identity to SEQ ID NO: 411 and a variable light chain (VL) region having an amino acid sequence with at least 80% identity to SEQ ID NO: 415.

429. The monospecific antibody of any one of claims 1-11 and 428, wherein the antibody comprises a variable heavy chain (VH) region having an amino acid sequence with at least 85% identity to SEQ ID NO: 411 and a variable light chain (VL) region having an amino acid sequence with at least 85% identity to SEQ ID NO: 415.

430. The monospecific antibody of any one of claims 1-11, 428, and 428, wherein the antibody comprises a variable heavy chain (VH) region having an amino acid sequence with at least 90% identity to SEQ ID NO: 411 and a variable light chain (VL) region having an amino acid sequence with at least 90% identity to SEQ ID NO: 415.

431. The monospecific antibody of any one of claims 1-11 and 428-430, wherein the antibody comprises a variable heavy chain (VH) region having an amino acid sequence with at least 95% identity to SEQ ID NO: 411 and a variable light chain (VL) region having an amino acid sequence with at least 95% identity to SEQ ID NO: 415.

432. The monospecific antibody of any one of claims 1-11 and 428-431, wherein the antibody comprises a variable heavy chain (VH) region having an amino acid sequence 100% identical to the amino acid sequence of SEQ ID NO: 411 and a variable light chain (VL) region having an amino acid sequence 100% identical to the amino acid sequence of SEQ ID NO: 415.

433. The monospecific antibody of any one of claims 1-11 and 428-432, wherein the antibody comprises a VH comprising therein a complementarity determining region (CDR) H1 having an amino acid sequence 100% identical to the amino acid sequence of SEQ ID NO: 412, a CDR H2 having an amino acid sequence 100% identical to the amino acid sequence of SEQ ID NO: 413, and a CDR H3 having an amino acid sequence 100% identical to the amino acid sequence of SEQ ID NO: 414; and a VL comprising therein a complementarity determining region (CDR) L1 having an amino acid sequence 100% identical to the amino acid sequence of SEQ ID NO: 416, a CDR L2 having an amino acid sequence 100% identical to the amino acid sequence of DAF, and a CDR L3 having an amino acid sequence 100% identical to the amino acid sequence of SEQ ID NO: 417.

434. The monospecific antibody of any one of claims 1-11 and 428-433, wherein the antibody comprises: (a) a variable heavy chain (VH) region having an amino acid sequence with at least 80% identity to SEQ ID NO: 411 and comprising therein a complementarity determining region (CDR) H1 having an amino acid sequence 100% identical to the amino acid sequence of SEQ ID NO: 412, a CDR H2 having an amino acid sequence 100% identical to the amino acid sequence of SEQ ID NO: 413, and a CDR H3 having an amino acid sequence 100% identical to the amino acid sequence of SEQ ID NO: 414; a variable light chain (VL) region having an amino acid sequence with at least 80% identity to SEQ ID NO: 415 and comprising therein a complementarity determining region (CDR) L1 having an amino acid sequence 100% identical to the amino acid sequence of SEQ ID NO: 416, a CDR L2 having an amino acid sequence 100% identical to the amino acid sequence of DAF, and a CDR L3 having an amino acid sequence 100% identical to the amino acid sequence of SEQ ID NO: 417.

435. The monospecific antibody of any one of claims 1-11, wherein the antibody comprises a variable heavy chain (VH) region having an amino acid sequence with at least 80% identity to SEQ ID NO: 418 and a variable light chain (VL) region having an amino acid sequence with at least 80% identity to SEQ ID NO: 422.

436. The monospecific antibody of any one of claims 1-11 and 435, wherein the antibody comprises a variable heavy chain (VH) region having an amino acid sequence with at least 85% identity to SEQ ID NO: 418 and a variable light chain (VL) region having an amino acid sequence with at least 85% identity to SEQ ID NO: 422.

437. The monospecific antibody of any one of claims 1-11, 435, and 436, wherein the antibody comprises a variable heavy chain (VH) region having an amino acid sequence with at least 90% identity to SEQ ID NO: 418 and a variable light chain (VL) region having an amino acid sequence with at least 90% identity to SEQ ID NO: 422.

438. The monospecific antibody of any one of claims 1-11 and 435-437, wherein the antibody comprises a variable heavy chain (VH) region having an amino acid sequence with at least 95% identity to SEQ ID NO: 418 and a variable light chain (VL) region having an amino acid sequence with at least 95% identity to SEQ ID NO: 422.

439. The monospecific antibody of any one of claims 1-11 and 435-438, wherein the antibody comprises a variable heavy chain (VH) region having an amino acid sequence 100% identical to the amino acid sequence of SEQ ID NO: 418 and a variable light chain (VL) region having an amino acid sequence 100% identical to the amino acid sequence of SEQ ID NO: 422.

440. The monospecific antibody of any one of claims 1-11 and 435-439, wherein the antibody comprises a VH comprising therein a complementarity determining region (CDR) H1 having an amino acid sequence 100% identical to the amino acid sequence of SEQ ID NO: 419, a CDR H2 having an amino acid sequence 100% identical to the amino acid sequence of SEQ ID NO: 420, and a CDR H3 having an amino acid sequence 100% identical to the amino acid sequence of SEQ ID NO: 421; and a VL comprising therein a complementarity determining region (CDR) L1 having an amino acid sequence 100% identical to the amino acid sequence of SEQ ID NO: 423, a CDR L2 having an amino acid sequence 100% identical to the amino acid sequence of DAS, and a CDR L3 having an amino acid sequence 100% identical to the amino acid sequence of SEQ ID NO: 424.

441. The monospecific antibody of any one of claims 1-11 and 435-440, wherein the antibody comprises: (a) a variable heavy chain (VH) region having an amino acid sequence with at least 80% identity to SEQ ID NO: 418 and comprising therein a complementarity determining region (CDR) H1 having an amino acid sequence 100% identical to the amino acid sequence of SEQ ID NO: 419, a CDR H2 having an amino acid sequence 100% identical to the amino acid sequence of SEQ ID NO: 420, and a CDR H3 having an amino acid sequence 100% identical to the amino acid sequence of SEQ ID NO: 421; a variable light chain (VL) region having an amino acid sequence with at least 80% identity to SEQ ID NO: 422 and comprising therein a complementarity determining region (CDR) L1 having an amino acid sequence 100% identical to the amino acid sequence of SEQ ID NO: 423, a CDR L2 having an amino acid sequence 100% identical to the amino acid sequence of DAS, and a CDR L3 having an amino acid sequence 100% identical to the amino acid sequence of SEQ ID NO: 424.

442. The monospecific antibody of any one of claims 1-11, wherein the antibody comprises a variable heavy chain (VH) region having an amino acid sequence with at least 80% identity to SEQ ID NO: 425 and a variable light chain (VL) region having an amino acid sequence with at least 80% identity to SEQ ID NO: 429.

443. The monospecific antibody of any one of claims 1-11 and 442, wherein the antibody comprises a variable heavy chain (VH) region having an amino acid sequence with at least 85% identity to SEQ ID NO: 425 and a variable light chain (VL) region having an amino acid sequence with at least 85% identity to SEQ ID NO: 429.

444. The monospecific antibody of any one of claims 1-11, 442, and 443, wherein the antibody comprises a variable heavy chain (VH) region having an amino acid sequence with at least 90% identity to SEQ ID NO: 425 and a variable light chain (VL) region having an amino acid sequence with at least 90% identity to SEQ ID NO: 429.

445. The monospecific antibody of any one of claims 1-11 and 442-444, wherein the antibody comprises a variable heavy chain (VH) region having an amino acid sequence with at least 95% identity to SEQ ID NO: 425 and a variable light chain (VL) region having an amino acid sequence with at least 95% identity to SEQ ID NO: 429.

446. The monospecific antibody of any one of claims 1-11 and 442-445, wherein the antibody comprises a variable heavy chain (VH) region having an amino acid sequence 100% identical to the amino acid sequence of SEQ ID NO: 425 and a variable light chain (VL) region having an amino acid sequence 100% identical to the amino acid sequence of SEQ ID NO: 429.

447. The monospecific antibody of any one of claims 1-11 and 442-446, wherein the antibody comprises a VH comprising therein a complementarity determining region (CDR) H1 having an amino acid sequence 100% identical to the amino acid sequence of SEQ ID NO: 426, a CDR H2 having an amino acid sequence 100% identical to the amino acid sequence of SEQ ID NO: 427, and a CDR H3 having an amino acid sequence 100% identical to the amino acid sequence of SEQ ID NO: 428; and a VL comprising therein a complementarity determining region (CDR) L1 having an amino acid sequence 100% identical to the amino acid sequence of SEQ ID NO: 430, a CDR L2 having an amino acid sequence 100% identical to the amino acid sequence of DAS, and a CDR L3 having an amino acid sequence 100% identical to the amino acid sequence of SEQ ID NO: 431.

448. The monospecific antibody of any one of claims 1-11 and 442-447, wherein the antibody comprises: (a) a variable heavy chain (VH) region having an amino acid sequence with at least 80% identity to SEQ ID NO: 425 and comprising therein a complementarity determining region (CDR) H1 having an amino acid sequence 100% identical to the amino acid sequence of SEQ ID NO: 426, a CDR H2 having an amino acid sequence 100% identical to the amino acid sequence of SEQ ID NO: 427, and a CDR H3 having an amino acid sequence 100% identical to the amino acid sequence of SEQ ID NO: 428; a variable light chain (VL) region having an amino acid sequence with at least 80% identity to SEQ ID NO: 429 and comprising therein a complementarity determining region (CDR) L1 having an amino acid sequence 100% identical to the amino acid sequence of SEQ ID NO: 430, a CDR L2 having an amino acid sequence 100% identical to the amino acid sequence of DAS, and a CDR L3 having an amino acid sequence 100% identical to the amino acid sequence of SEQ ID NO: 431.

449. The monospecific antibody of any one of claims 1-11, wherein the antibody comprises a variable heavy chain (VH) region having an amino acid sequence with at least 80% identity to SEQ ID NO: 432 and a variable light chain (VL) region having an amino acid sequence with at least 80% identity to SEQ ID NO: 436.

450. The monospecific antibody of any one of claims 1-11 and 449, wherein the antibody comprises a variable heavy chain (VH) region having an amino acid sequence with at least 85% identity to SEQ ID NO: 432 and a variable light chain (VL) region having an amino acid sequence with at least 85% identity to SEQ ID NO: 436.

451. The monospecific antibody of any one of claims 1-11, 449, and 450, wherein the antibody comprises a variable heavy chain (VH) region having an amino acid sequence with at least 90% identity to SEQ ID NO: 432 and a variable light chain (VL) region having an amino acid sequence with at least 90% identity to SEQ ID NO: 436.

452. The monospecific antibody of any one of claims 1-11 and 449-451, wherein the antibody comprises a variable heavy chain (VH) region having an amino acid sequence with at least 95% identity to SEQ ID NO: 432 and a variable light chain (VL) region having an amino acid sequence with at least 95% identity to SEQ ID NO: 436.

453. The monospecific antibody of any one of claims 1-11 and 449-452, wherein the antibody comprises a variable heavy chain (VH) region having an amino acid sequence 100% identical to the amino acid sequence of SEQ ID NO: 432 and a variable light chain (VL) region having an amino acid sequence 100% identical to the amino acid sequence of SEQ ID NO: 436.

454. The monospecific antibody of any one of claims 1-11 and 449-453, wherein the antibody comprises a VH comprising therein a complementarity determining region (CDR) H1 having an amino acid sequence 100% identical to the amino acid sequence of SEQ ID NO: 433, a CDR H2 having an amino acid sequence 100% identical to the amino acid sequence of SEQ ID NO: 434, and a CDR H3 having an amino acid sequence 100% identical to the amino acid sequence of SEQ ID NO: 435; and a VL comprising therein a complementarity determining region (CDR) L1 having an amino acid sequence 100% identical to the amino acid sequence of SEQ ID NO: 437, a CDR L2 having an amino acid sequence 100% identical to the amino acid sequence of GAS, and a CDR L3 having an amino acid sequence 100% identical to the amino acid sequence of SEQ ID NO: 438.

455. The monospecific antibody of any one of claims 1-11 and 449-454, wherein the antibody comprises: (a) a variable heavy chain (VH) region having an amino acid sequence with at least 80% identity to SEQ ID NO: 432 and comprising therein a complementarity determining region (CDR) H1 having an amino acid sequence 100% identical to the amino acid sequence of SEQ ID NO: 433, a CDR H2 having an amino acid sequence 100% identical to the amino acid sequence of SEQ ID NO: 434, and a CDR H3 having an amino acid sequence 100% identical to the amino acid sequence of SEQ ID NO: 435; a variable light chain (VL) region having an amino acid sequence with at least 80% identity to SEQ ID NO: 436 and comprising therein a complementarity determining region (CDR) L1 having an amino acid sequence 100% identical to the amino acid sequence of SEQ ID NO: 437, a CDR L2 having an amino acid sequence 100% identical to the amino acid sequence of GAS, and a CDR L3 having an amino acid sequence 100% identical to the amino acid sequence of SEQ ID NO: 438.

456. The monospecific antibody of any one of claims 1-11, wherein the antibody comprises a variable heavy chain (VH) region having an amino acid sequence with at least 80% identity to SEQ ID NO: 439 and a variable light chain (VL) region having an amino acid sequence with at least 80% identity to SEQ ID NO: 443.

457. The monospecific antibody of any one of claims 1-11 and 456, wherein the antibody comprises a variable heavy chain (VH) region having an amino acid sequence with at least 85% identity to SEQ ID NO: 439 and a variable light chain (VL) region having an amino acid sequence with at least 85% identity to SEQ ID NO: 443.

458. The monospecific antibody of any one of claims 1-11, 456, and 457, wherein the antibody comprises a variable heavy chain (VH) region having an amino acid sequence with at least 90% identity to SEQ ID NO: 439 and a variable light chain (VL) region having an amino acid sequence with at least 90% identity to SEQ ID NO: 443.

459. The monospecific antibody of any one of claims 1-11 and 456-458, wherein the antibody comprises a variable heavy chain (VH) region having an amino acid sequence with at least 95% identity to SEQ ID NO: 439 and a variable light chain (VL) region having an amino acid sequence with at least 95% identity to SEQ ID NO: 443.

460. The monospecific antibody of any one of claims 1-11 and 456-459, wherein the antibody comprises a variable heavy chain (VH) region having an amino acid sequence 100% identical to the amino acid sequence of SEQ ID NO: 439 and a variable light chain (VL) region having an amino acid sequence 100% identical to the amino acid sequence of SEQ ID NO: 443.

461. The monospecific antibody of any one of claims 1-11 and 456-460, wherein the antibody comprises a VH comprising therein a complementarity determining region (CDR) H1 having an amino acid sequence 100% identical to the amino acid sequence of SEQ ID NO: 440, a CDR H2 having an amino acid sequence 100% identical to the amino acid sequence of SEQ ID NO: 441, and a CDR H3 having an amino acid sequence 100% identical to the amino acid sequence of SEQ ID NO: 442; and a VL comprising therein a complementarity determining region (CDR) L1 having an amino acid sequence 100% identical to the amino acid sequence of SEQ ID NO: 444, a CDR L2 having an amino acid sequence 100% identical to the amino acid sequence of AAS, and a CDR L3 having an amino acid sequence 100% identical to the amino acid sequence of SEQ ID NO: 445.

462. The monospecific antibody of any one of claims 1-11 and 456-461, wherein the antibody comprises: (a) a variable heavy chain (VH) region having an amino acid sequence with at least 80% identity to SEQ ID NO: 439 and comprising therein a complementarity determining region (CDR) H1 having an amino acid sequence 100% identical to the amino acid sequence of SEQ ID NO: 440, a CDR H2 having an amino acid sequence 100% identical to the amino acid sequence of SEQ ID NO: 441, and a CDR H3 having an amino acid sequence 100% identical to the amino acid sequence of SEQ ID NO: 442; a variable light chain (VL) region having an amino acid sequence with at least 80% identity to SEQ ID NO: 443 and comprising therein a complementarity determining region (CDR) L1 having an amino acid sequence 100% identical to the amino acid sequence of SEQ ID NO: 444, a CDR L2 having an amino acid sequence 100% identical to the amino acid sequence of AAS, and a CDR L3 having an amino acid sequence 100% identical to the amino acid sequence of SEQ ID NO: 445.

463. The monospecific antibody of any one of claims 1-11, wherein the antibody comprises a variable heavy chain (VH) region having an amino acid sequence with at least 80% identity to SEQ ID NO: 446 and a variable light chain (VL) region having an amino acid sequence with at least 80% identity to SEQ ID NO: 450.

464. The monospecific antibody of any one of claims 1-11 and 463, wherein the antibody comprises a variable heavy chain (VH) region having an amino acid sequence with at least 85% identity to SEQ ID NO: 446 and a variable light chain (VL) region having an amino acid sequence with at least 85% identity to SEQ ID NO: 450.

465. The monospecific antibody of any one of claims 1-11, 463, and 464, wherein the antibody comprises a variable heavy chain (VH) region having an amino acid sequence with at least 90% identity to SEQ ID NO: 446 and a variable light chain (VL) region having an amino acid sequence with at least 90% identity to SEQ ID NO: 450.

466. The monospecific antibody of any one of claims 1-11 and 463-465, wherein the antibody comprises a variable heavy chain (VH) region having an amino acid sequence with at least 95% identity to SEQ ID NO: 446 and a variable light chain (VL) region having an amino acid sequence with at least 95% identity to SEQ ID NO: 450.

467. The monospecific antibody of any one of claims 1-11 and 463-466, wherein the antibody comprises a variable heavy chain (VH) region having an amino acid sequence 100% identical to the amino acid sequence of SEQ ID NO: 446 and a variable light chain (VL) region having an amino acid sequence 100% identical to the amino acid sequence of SEQ ID NO: 450.

468. The monospecific antibody of any one of claims 1-11 and 463-467, wherein the antibody comprises a VH comprising therein a complementarity determining region (CDR) H1 having an amino acid sequence 100% identical to the amino acid sequence of SEQ ID NO: 447, a CDR H2 having an amino acid sequence 100% identical to the amino acid sequence of SEQ ID NO: 448, and a CDR H3 having an amino acid sequence 100% identical to the amino acid sequence of SEQ ID NO: 449; and a VL comprising therein a complementarity determining region (CDR) L1 having an amino acid sequence 100% identical to the amino acid sequence of SEQ ID NO: 451, a CDR L2 having an amino acid sequence 100% identical to the amino acid sequence of DNF, and a CDR L3 having an amino acid sequence 100% identical to the amino acid sequence of SEQ ID NO: 452.

469. The monospecific antibody of any one of claims 1-11 and 463-468, wherein the antibody comprises: (a) a variable heavy chain (VH) region having an amino acid sequence with at least 80% identity to SEQ ID NO: 446 and comprising therein a complementarity determining region (CDR) H1 having an amino acid sequence 100% identical to the amino acid sequence of SEQ ID NO: 447, a CDR H2 having an amino acid sequence 100% identical to the amino acid sequence of SEQ ID NO: 448, and a CDR H3 having an amino acid sequence 100% identical to the amino acid sequence of SEQ ID NO: 449; a variable light chain (VL) region having an amino acid sequence with at least 80% identity to SEQ ID NO: 450 and comprising therein a complementarity determining region (CDR) L1 having an amino acid sequence 100% identical to the amino acid sequence of SEQ ID NO: 451, a CDR L2 having an amino acid sequence 100% identical to the amino acid sequence of DNF, and a CDR L3 having an amino acid sequence 100% identical to the amino acid sequence of SEQ ID NO: 452.

470. The monospecific antibody of any one of claims 1-11, wherein the antibody comprises a variable heavy chain (VH) region having an amino acid sequence with at least 80% identity to SEQ ID NO: 61 and a variable light chain (VL) region having an amino acid sequence with at least 80% identity to SEQ ID NO: 65.

471. The monospecific antibody of any one of claims 1-11, and 470, wherein the antibody comprises a variable heavy chain (VH) region having an amino acid sequence with at least 85% identity to SEQ ID NO: 61 and a variable light chain (VL) region having an amino acid sequence with at least 85% identity to SEQ ID NO: 65.

472. The monospecific antibody of any one of claims 1-11, 470 and 470-471, wherein the antibody comprises a variable heavy chain (VH) region having an amino acid sequence with at least 90% identity to SEQ ID NO: 61 and a variable light chain (VL) region having an amino acid sequence with at least 90% identity to SEQ ID NO: 65.

473. The monospecific antibody of any one of claims 1-11 and 470-472, wherein the antibody comprises a variable heavy chain (VH) region having an amino acid sequence with at least 95% identity to SEQ ID NO: 61 and a variable light chain (VL) region having an amino acid sequence with at least 95% identity to SEQ ID NO: 65.

474. The monospecific antibody of any one of claims 1-11 and 470-473, wherein the antibody comprises a variable heavy chain (VH) region having an amino acid sequence 100% identical to the amino acid sequence of SEQ ID NO: 61 and a variable light chain (VL) region having an amino acid sequence 100% identical to the amino acid sequence of SEQ ID NO: 65.

475. The monospecific antibody of any one of claims 1-11 and 470-474, wherein the antibody comprises a VH comprising therein a complementarity determining region (CDR) H1 having an amino acid sequence 100% identical to the amino acid sequence of SEQ ID NO: 62, a CDR H2 having an amino acid sequence 100% identical to the amino acid sequence of SEQ ID NO: 63, and a CDR H3 having an amino acid sequence 100% identical to the amino acid sequence of SEQ ID NO: 64; and a VL comprising therein a complementarity determining region (CDR) L1 having an amino acid sequence 100% identical to the amino acid sequence of SEQ ID NO: 66, a CDR L2 having an amino acid sequence 100% identical to the amino acid sequence of DAS, and a CDR L3 having an amino acid sequence 100% identical to the amino acid sequence of SEQ ID NO: 67.

476. The monospecific antibody of any one of claims 1-11 and 470-475, wherein the antibody comprises: (a) a variable heavy chain (VH) region having an amino acid sequence with at least 80% identity to SEQ ID NO: 61 and comprising therein a complementarity determining region (CDR) H1 having an amino acid sequence 100% identical to the amino acid sequence of SEQ ID NO: 62, a CDR H2 having an amino acid sequence 100% identical to the amino acid sequence of SEQ ID NO: 63, and a CDR H3 having an amino acid sequence 100% identical to the amino acid sequence of SEQ ID NO: 64; a variable light chain (VL) region having an amino acid sequence with at least 80% identity to SEQ ID NO: 65 and comprising therein a complementarity determining region (CDR) L1 having an amino acid sequence 100% identical to the amino acid sequence of SEQ ID NO: 66, a CDR L2 having an amino acid sequence 100% identical to the amino acid sequence of DAS, and a CDR L3 having an amino acid sequence 100% identical to the amino acid sequence of SEQ ID NO: 67.

477. The monospecific antibody of any one of claims 1-476, wherein the antibody binds to one or more amino acid residues of the VWF CK domain selected from D2726, R2730, K2735, E2744, D2746, K2757, D2763, and T2789.

478. The monospecific antibody of any one of claims 1-477, wherein the antibody binds to at least the following amino acid residues of the VWF CK domain: D2726, R2730, D2746, and K2757.

479. The monospecific antibody of any one of claims 1-477, wherein the antibody binds to at least the following amino acid residues of the VWF CK domain: R2730, K2735, E2744, and D2746.

480. The monospecific antibody of any one of claims 1-477, wherein the antibody binds to at least the following amino acid residues of the VWF CK domain: K2735, D2746, and K2757.

481. The monospecific antibody of any one of claims 1-477, wherein the antibody binds to at least the following amino acid residues of the VWF CK domain: R2730, D2746, and K2757.

482. The monospecific antibody of any one of claims 1-477, wherein the antibody binds to at least the following amino acid residues of the VWF CK domain: K2735, E2744, D2746, and D2763.

483. The monospecific antibody of any one of claims 1-477, wherein the antibody binds to at least the following amino acid residues of the VWF CK domain: R2730, K2735, E2744, D2746, K2757, and D2763.

484. The monospecific antibody of any one of claims 1-477, wherein the antibody binds to at least amino acid residue K2735 of the VWF CK domain.

485. The monospecific antibody of any one of claims 1-477, wherein the antibody binds to at least the following amino acid residues of the VWF CK domain: D2726, K2735, and K2757.

486. The monospecific antibody of any one of claims 1-477, wherein the antibody binds to at least the following amino acid residues of the VWF CK domain: K2735 and D2763.

487. The monospecific antibody of any one of claims 1-486, wherein binding of the antibody to the VWF protein does not result in a change in function of the VWF protein.

488. The monospecific antibody of any one of claims 1-487, wherein binding of the antibody to the VWF protein does not inhibit VWF protein binding to one or more binding partners.

489. The monospecific antibody of claim 488, wherein the one or more binding partners are selected from the group consisting of Factor VIII, collagen, glycoprotein Ib (gp1b), glycoprotein IIb/IIIa (gpIIb/IIIa), and ADAM Metallopeptidase With Thrombospondin Type 1 Motif 13 (ADAMTS13).

490. The monospecific antibody of any one of claims 487-489, wherein binding of the antibody to the VWF protein does not inhibit VWF protein binding to platelet cells.

491. The monospecific antibody of any one of claims 1-490, wherein the accumulation of the VWF protein in blood plasma results in a blood plasma level of the VWF protein that is at least 1.3-fold, 1.4-fold, 1.5-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold greater as compared to a blood plasma level of the VWF protein in the absence of the antibody.

492. The monospecific antibody of any one of claims 1-490, wherein the accumulation of the VWF protein in blood plasma results in a blood plasma level of the VWF protein that is at least 5%, 10%, 20%, 30%, 40%, 50%, 60%, 70%, 80%, 90%, 100%, 150%, 200%, 250%, 300%, 350%, 400%, 450%, 500%, 550%, 600%, 650%, 700%, 750%, 800%, 850%, 900%, 950%, 1,000%, 1,500%, 2,000%, 2,500%, 3,000%, 3,500%, 4,000%, 4,500%, 5,000%, 5,500%, 6,000%, 6,500%, 7,000%, 7,500%, 8,000%, 8,500%, 9,000%, 9,500%, 10,000%, or greater as compared to a blood plasma level of the VWF protein in the absence of the antibody.

493. The monospecific antibody of any one of claims 1-492, wherein binding of the antibody to the VWF protein in blood plasma increases a blood plasma half-life of the VWF protein as compared to a blood plasma half-life of the VWF protein in the absence of the antibody.

494. The monospecific antibody of claim 493, wherein binding of the antibody to the VWF protein in blood plasma increases the blood plasma half-life of the VWF protein by at least 1.5-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold or more as compared to the blood plasma half-life of the VWF protein in the absence of the antibody.

495. The monospecific antibody of claim 493, wherein binding of the antibody to the VWF protein in blood plasma increases the blood plasma half-life of the VWF protein by at least 5%, 10%, 20%, 30%, 40%, 50%, 60%, 70%, 80%, 90%, 100%, 150%, 200%, 250%, 300%, 350%, 400%, 450%, 500%, 550%, 600%, 650%, 700%, 750%, 800%, 850%, 900%, 950%, 1,000%, 1,500%, 2,000%, 2,500%, 3,000%, 3,500%, 4,000%, 4,500%, 5,000%, 5,500%, 6,000%, 6,500%, 7,000%, 7,500%, 8,000%, 8,500%, 9,000%, 9,500%, or 10,000% or more as compared to the blood plasma half-life of the VWF protein in the absence of the antibody.

496. The monospecific antibody of any one of claims 1-495, wherein binding of the antibody to the VWF protein in blood plasma results in accumulation of a Factor VIII (FVIII) protein in blood plasma as compared to a blood plasma level of a FVIII protein in the absence of the antibody.

497. The monospecific antibody of claim 496, wherein the accumulation of the FVIII protein in blood plasma results in a blood plasma level of the Factor VIII protein that is at least 1.3-fold, 1.4-fold, 1.5-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold greater as compared to a blood plasma level of the FVIII protein in the absence of the antibody.

498. The monospecific antibody of claim 496, wherein the accumulation of the FVIII protein in blood plasma results in a blood plasma level of the FVIII protein that is at least 5%, 10%, 20%, 30%, 40%, 50%, 60%, 70%, 80%, 90%, 100%, 150%, 200%, 250%, 300%, 350%, 400%, 450%, 500%, 550%, 600%, 650%, 700%, 750%, 800%, 850%, 900%, 950%, 1,000%, 1,500%, 2,000%, 2,500%, 3,000%, 3,500%, 4,000%, 4,500%, 5,000%, 5,500%, 6,000%, 6,500%, 7,000%, 7,500%, 8,000%, 8,500%, 9,000%, 9,500%, 10,000%, or greater as compared to the blood plasma level of a FVIII protein in the absence of the antibody.

499. The monospecific antibody of any one of claims 1-598, wherein binding of the antibody to the VWF protein in blood plasma increases the blood plasma half-life of a FVIII protein as compared to the blood plasma half-life of the FVIII protein in the absence of the antibody.

500. The monospecific antibody of claim 499, wherein binding of the antibody to the VWF protein in blood plasma increases the blood plasma half-life of the FVIII protein by at least 1.5-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold or more as compared to the blood plasma half-life of the FVIII protein in the absence of the antibody.

501. The monospecific antibody of claim 499, wherein binding of the antibody to the VWF protein in blood plasma increases the blood plasma half-life of the FVIII protein by at least 5%, 10%, 20%, 30%, 40%, 50%, 60%, 70%, 80%, 90%, 100%, 150%, 200%, 250%, 300%, 350%, 400%, 450%, 500%, 550%, 600%, 650%, 700%, 750%, 800%, 850%, 900%, 950%, 1,000%, 1,500%, 2,000%, 2,500%, 3,000%, 3,500%, 4,000%, 4,500%, 5,000%, 5,500%, 6,000%, 6,500%, 7,000%, 7,500%, 8,000%, 8,500%, 9,000%, 9,500%, or 10,000% or more as compared to the blood plasma half-life of the FVIII protein in the absence of the antibody.

502. The monospecific antibody of any one of claims 1-501, wherein the antibody binds specifically to a VWF homodimer comprising two VWF monomers linked tail-to-tail via CK-domain joining in a 1:1 stoichiometry.

503. The monospecific antibody of any one of claims 1-501, wherein the antibody binds specifically to a VWF homodimer comprising two VWF monomers linked tail-to-tail via CK-domain joining in a 2:1 stoichiometry.

504. The monospecific antibody of any one of claims 1-503, wherein the antibody binds to the CK domain of the VWF protein with a dissociation constant (K_{d}) of less than 100 nM, 90 nM, 80 nM, 70 nM, 60 nM, 50 nM, 40 nM, 30 nM, 20 nM, 10 nM, 7 nM, 5 nM, 4 nM, 3 nM, 2 nM, 1 nM, 0.5 nM, 0.25 nM, 0.1 nM, 0.075 nM, 0.05 nM, 0.025 nM, 0.01 nM, or less.

505. The monospecific antibody of any one of claims 1-504, wherein the antibody is cross-reactive with a VWF protein of Macaca fascicularis, Mus musculus, and/or Canis familiaris.

506. The monospecific antibody of claim 505, wherein the antibody exhibits an affinity to the VWF protein of Macaca fascicularis, Mus musculus, and/or Canis familiaris that is at most 10-fold lower than an affinity of the antibody to a human VWF protein.

507. The monospecific antibody of any one of claims 1-506, wherein the antibody is a human antibody, humanized antibody, or chimeric antibody.

508. The monospecific antibody of any one of claims 1-507, wherein the CK domain has an amino acid sequence of SEQ ID NO: 5 or SEQ ID NO: 6.

509. A complex comprising a VWF protein and a monospecific antibody of any one of claims 1-508.

510. A pharmaceutical composition comprising the monospecific antibody of any one of claims 1-508 and a pharmaceutically acceptable carrier, diluent, or excipient.

511. The pharmaceutical composition of claim 510, wherein the pharmaceutically acceptable carrier, diluent, or excipient is selected from the group consisting of a stabilizer, buffer, surfactant, filler, solvent, tonicity or osmolarity adjusting agent, antioxidant, adjuvant, and antimicrobial agent.

512. A method of treating a blood disorder characterized by excessive bleeding in a subject in need thereof, comprising administering to the subject the monospecific antibody of any one of claims 1-508 or the pharmaceutical composition of claim 510 or 511.

513. The method of claim 512, wherein the blood disorder is selected from the group consisting of von Willebrand disease (VWD), Heydes syndrome, hemophilia A, disorders of platelet function, and connective tissue disorders.

514. The method of claim 513, wherein the VWD is congenital VWD (cVWD) or acquired VWD (aVWD).

515. The method of claim 513 or 514, wherein the VWD is type 1, type 2A, type 2B, type 2M, type 2N, or type 3.

516. The method of any one of claims 512-515, wherein the composition treats one or more symptoms of the blood disorder selected from the group consisting of epistaxis, cutaneous bleeding, bleeding from minor wounds, oral-cavity bleeding, angiodysplasia, gastrointestinal bleeding, bleeding from tooth extraction, postoperative bleeding, heavy menstrual bleeding, obstetric hemorrhage, hematuria, muscle hematoma, joint bleeding, visceral bleeding, and central nervous system (CNS) bleeding.

517. The method of any one of claims 512-516, wherein the composition or pharmaceutical composition promotes platelet adhesion and/or aggregation in the subject.

518. A method of accumulating a VWF protein in blood plasma of a subject in need thereof, comprising administering to the subject a composition comprising the monospecific antibody of any one of claims 1-508 or the pharmaceutical composition of claim 510 or 511.

519. A method of accumulating a FVIII protein in blood plasma of a subject in need thereof, comprising administering to the subject a composition comprising the monospecific antibody of any one of claims 1-508 or the pharmaceutical composition of claim 510 or 511.

520. A method of increasing blood plasma half-life of a VWF protein in a subject in need thereof, comprising administering to the subject a composition comprising a monospecific antibody of any one of claims 1-508 or the pharmaceutical composition of claim 510 or 511.

521. The method of any one of claims 512-520, wherein the composition is a pharmaceutical composition comprising the monospecific antibody and a pharmaceutically acceptable carrier, diluent, or excipient.

522. The method of claim 521, wherein the pharmaceutically acceptable carrier, diluent, or excipient is selected from the group consisting of a stabilizer, buffer, surfactant, filler, solvent, tonicity or osmolarity adjusting agent, antioxidant, adjuvant, and antimicrobial agent.

523. The method of any one of claims 512-522, wherein the composition or the pharmaceutical composition is administered in combination with one or more additional therapeutic modalities.

524. The method of claim 523, wherein the one or more additional therapeutic modalities are selected from the group consisting of desmopressin (DDAVP), tranexamic acid (TXA), thalidomide, tamoxifen, ocreotide, a concentrate of plasma-derived VWF protein, a concentrate of VWF protein in complex with FVIII protein, a concentrate of recombinant VWF protein, a concentrate of FVIII protein, hormonal treatment, gene therapy, blood transfusion, platelet transfusion, surgery, and cauterization.

525. The method of claim 524, wherein the gene therapy is FVIII gene therapy.

526. The method of any one of claims 523-525, wherein the one or more additional therapeutic modalities are administered to the subject prior to, concurrently with, or subsequent to administration of the composition or the pharmaceutical composition.

527. The method of any one of claims 512-526, wherein the subject is a human.

528. A kit comprising the monospecific antibody of any one of claims 1-508 or the pharmaceutical composition of claim 510 or 511, and a package insert instructing a user of the kit to perform the method of any one of claims 512-527.

529. A monospecific antibody of any one of claims 1-508 or the pharmaceutical composition of claim 510 or 511 for use as a medicament.

530. The monospecific antibody or the pharmaceutical composition of claim 529 for use in the treatment of a blood disorder characterized by excessive bleeding.

531. A monospecific antibody of any one of claims 1-508 or the pharmaceutical composition of claim 510 or 511 for use in the treatment of a blood disorder characterized by excessive bleeding.

532. The monospecific antibody or the pharmaceutical composition for use according to claim 530 or 531, wherein the blood disorder characterized by excessive bleeding is selected from the group consisting of VWD, Heydes syndrome, hemophilia A, disorders of platelet function, and connective tissue disorders.

533. The monospecific antibody or the pharmaceutical composition for use according to claim 532, wherein the VWD is cVWD or aVWD.

534. The monospecific antibody or the pharmaceutical composition for use according to claim 532 or 533, wherein the VWD is type 1, type 2A, type 2B, type 2M, type 2N, or type 3.


SEQUENCE LISTING (487 sequences)

SEQ ID NO: 1 - protein, 2813 aa, Homo sapiens
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDVQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCQDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLCDLAPEAPPPTLPPDMAQVTVGPGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICMDEDGNEKRPGDVWTLPDQCHTVTCQPDGQTLLKSHRVNCDRGLRPSCPNSQSPVKVEETCGCRWTCPCVCTGSSTRHIVTFDGQNFKLTGSCSYVLFQNKEQDLEVILHNGACSPGARQGCMKSIEVKHSALSVELHSDMEVTVNGRLVSVPYVGGNMEVNVYGAIMHEVRFNHLGHIFTFTPQNNEFQLQLSPKTFASKTYGLCGICDENGANDFMLRDGTVTTDWKTLVQEWTVQRPGQTCQPILEEQCLVPDSSHCQVLLLPLFAECHKVLAPATFYAICQQDSCHQEQVCEVIASYAHLCRTNGVCVDWRTPDFCAMSCPPSLVYNHCEHGCPRHCDGNVSSCGDHPSEGCFCPPDKVMLEGSCVPEEACTQCIGEDGVQHQFLEAWVPDHQPCQICTCLSGRKVNCTTQPCPTAKAPTCGLCEVARLRQNADQCCPEYECVCDPVSCDLPPVPHCERGLQPTLTNPGECRPNFTCACRKEECKRVSPPSCPPHRLPTLRKTQCCDEYECACNCVNSTVSCPLGYLASTATNDCGCTTTTCLPDKVCVHRSTIYPVGQFWEEGCDVCTCTDMEDAVMGLRVAQCSQKPCEDSCRSGFTYVLHEGECCGRCLPSACEVVTGSPRGDSQSSWKSVGSQWASPENPCLINECVRVKEEVFIQQRNVSCPQLEVPVCPSGFQLSCKTSACCPSCRCERMEACMLNGTVIGPGKTVMIDVCTTCRCMVQVGVISGFKLECRKTTCNPCPLGYKEENNTGECCGRCLPTACTIQLRGGQIMTLKRDETLQDGCDTHFCKVNERGEYFWEKRVTGCPPFDEHKCLAEGGKIMKIPGTCCDTCEEPECNDITARLQYVKVGSCKSEVEVDIHYCQGKCASKAMYSIDINDVQDQCSCCSPTRTEPMQVALHCTNGSVVYHEVLNAMECKCSPRKCSK

SEQ ID NO: 2 - protein, 2809 aa, Macaca fascicularis
MIPARLAGVLLALALVLPGTLCAEGTRGRSSMARCSLFGSDFINTFDGSMYSFAGYCSYLLAGDCQKRSFSIIGDFQNGKRVSLSVYLGEFFDIHLFVNGTVIQGDKSVSMPYASKGLYLETEAGYYKLSGEAYGFVARIDGSGNFQVLLSDRYFNKTCGLCGNFNIFAEDDFMTQEGTVTSDPYDFANSWALSSGEQWCERASPPSSSCNISSGEVQKGLWEQCQLLKSTSVFARCHPLVDPEPFVALCEKTSCECAGGLECTCPAFLEYTRTCAQEGMVLYGWTDHSACSPVCPAGMEYKQCVSPCARTCQSLHINEVCQERCVDGCSCPEGQLLDEGLCVESTECPCMHSGKRYPPGASLSRDCNTCICRNSQWICSNEECPGECLVTGQSHFKSFDNRYFTFSGICQYLLARDCQDHSFSIVIETVQCADDPDAVCTRSVTVRLPGLHNSLVKLKHGGGVAMDGQDVQLPLLKGDLRIQHSVTASVRLSYGEDLQMDWDGRGRLLVKLSPVYAGKTCGLCGNYNGNQGDDFLTPSGLAEPRVEDFGNAWKLHADCQDLQKQHSDPCALNPRMTRFSEEACAVLTSPTFEACHRAVSPLPYLRNCRYDVCSCSDGRECLCGALASYAACAGGVVAWEPGRCELKCPKGQVYLQCGTPCNLTCRALSYPDEECSEACLEGCFCPPGLYMDEMGDCVPKAQCPCYYDGEIFQPEDIFSDHHTMCYCEDGFMHCTMSGVPGSLLPDAVLSSPLSHRSKRSLSCRPPMVKLVCPADNPRAEGLECAKTCQNYDLECMSMGCVSGCLCPPGMVRHENRCVALERCPCLHQGKEYAPGEAVKIDCNTCVCRDRKWNCTDHVCDATCSMIGMAHYLTFDGLKYMFPGECQYVLVQDYCGGNPGTFRILVGNEGCSHPSVKCKKHVTILVEGGEIELFDGEVNVKRPMKDETHFEVVESGRYIILLLGKAISVVWDRHLSISVVLKQTYQEKVCGLCGNFDGIQNNDLTSSNLQVEEDPVDFGNSWKVSSQCADTRKVPLDSSPATCHNNLMKQTMVDSSCRILTSDVFQDCNKLVDPEPFLDVCIYDTCSCESIGDCACFCDTIAAYAHVCAQHGKVVTWRTATLCPQSCEERNLRENGYECEWRYNSCAPACRVTCQHPEPLACPVQCVEGCHAHCPPGKILDELLQTCVSPEDCPVCEAAGRRFASGKKVTLNPSDPEHCQICHCDGVNLTCEACEEPGGLVVPPTDAPVSPTTPYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFGKIDRPEASRIALLLMASQEPQRMSRNFVRYVQGLKKKKVIVIPVGIGPHANLKQIRLIEKQSPENKAFVLSGVDELEQQRDEIVSYLCDLAPEAPPPTLPPDMAQVTVGSGLLGVSTLGPKRNSMVLDVAFVLEGSDKIGEADFNRSKEFMEEVIQRMDVGQDGIHVTVLQYSYTVAVEYPFSEAQSKGDILQRVREIRYQGGNRTNTGLALQYLSDHSFLVSQGDREQAPNLVYMVTGNPASDEIKRLPGDIQVVPIGVGPHANVQELERIGWPNAPILIQDFETLPREAPDLVLQSCCSGEGLKIPTLSPAPDCSQPLDVILLLDGSSSFPAAYFDEMKSFAKAFISKANIGPHLTQVSVLQYGSITTIDVPWNVAPEKAHLLSLVDVMQREGGPSQIGGALGFAVRYLTSEMHGARPGASKAVVILVMDVSVDAVDAAADAARSNRVAVFPIGIGDRYDAAQLRILAGPAGNSNVVKLQRIEDLPTMVTLGNSFLHKLCSGFVRICMDEDGNERRPGDIWTLPDQCHTVTCQPDGQTLLESHRVNCDRGLRPSCPNSQSPVKVEETCGCRWTCPCVCTGSSTRHIVTFDGQNFKLTGSCSYVLFQNKEQDLEVILHNGACSPAARQGCMKSIEVKHAALSVELHSDMEVTVNGRLVSVPYVGGNMEVNVYGAIMHEIRFNHLGHIFTFTPQNNEFQLQLSPKTFASKTYGLCGICDENGANDFMLRDGTVTTDWKTLVQEWTVQRPGQTCQPILEEQCLVPNSSQCQVLLSALFAECHKVLAPATFYAICQQDSCHREQVCEVIASYAHLCRTNGVCVDWRTPDFCAMSCPPSLVYNPCERGCPRHCNGNVSSCGDHPSEGCFCPPNKVMLEGSCVPEEACTQCIGEDGVQHQFLEAWVPDHQPCQICTCLSGRKANCTMQPCPTAKAPTCGLCEVARLRQNADQCCPEYECVCDPESCDLPPVPHCEGGLQPTLTNPGECRPNFTCACRKEECKRVSLPSCPPHRLPTLRKTQCCDEYECACNCVNSTVSCPLGYLASTATNDCGCTTTTCLPDKVCVHRSTIYPVGQFWEEGCDVCTCTDMEDAVMGLRVVQCSQKPCEDSCRSGFTYVPREGECCGRCLPSACEVVTGSPRGDSQSSWKSVGSHWASPENPCLINECVRVKEEVFVQQRNVSCPQLEVPVCPSGFQLSCKTSACCPSCRCEPVEACMLNGTMIGPGKSVMIDACTTCRCIVQVGSISGFKLECRKTICNPCPLGYKEEKNTGECCGRCLPTVCTIRLRGGQIMTLKRDETLQDGCDTHFCKVNERGEYFWEKRVTGCPPFDEHKCLAEGGKIKKIPGTCCDTCEEPECSDITARLQYVKVGSCKSEVEVDIHYCQGKCASKAMYSIDINDVQDQCSCCSPTRTEPMQVPLHCTNGSVVYHEVLNAMQCECSPRKCSK

SEQ ID NO: 3 - protein, 2813 aa, Canis lupus
MSPTRLVRVLLALALILPGKLCTKGTVGRSSMARCSLFGGDFINTFDESMYSFAGDCSYLLAGDCQEHSVSLIGGFQNGKRVSLSVYLGEFFDIHLFVNGTMLQGTQSISMPYASNGLYLEAEAGYYKLSSEAYGFVARIDGNGNFQVLLSDRYFNKTCGLCGNFNIFAEDDFRTQEGTLTSDPYDFANSWALSSGEQRCKRVSPPSSPCNVSSDEVQQVLWEQCQLLKSASVFARCHPLVDPEPFVALCERTLCTCVQGMECPCAVLLEYARACAQQGIVLYGWTDHSVCRPACPAGMEYKECVSPCTRTCQSLHVKEVCQEQCVDGCSCPEGQLLDEGHCVGSAECSCVHAGQRYPPGASLLQDCHTCICRNSLWICSNEECPGECLVTGQSHFKSFDNRYFTFSGVCHYLLAQDCQDHTFSVVIETVQCADDLDAVCTRSVTVRLPGHHNSLVKLKHGGGVSMDGQDIQIPLLQGDLRIQHTVMASVRLSYGEDLQMDWDGRGRLLVTLSPAYAGKTCGLCGNYNGNRGDDFVTPAGLAEPLVEDFGNAWKLLGACENLQKQHRDPCSLNPRQARFAEEACALLTSSKFEPCHRAVGPQPYVQNCRYDVCSCSDGRDCLCSAVANYAAACARRGVHIAWREPGFCALSCPQGQVYLQCGTPCNMTCRSLSYPEEDCNEVCLEGCFCPPGLYLDERGDCVPKAQCPCYYDGEIFQPEDIFSDHHTMCYCEDGFMHCTTSGGLGSLLPNPVLSSPRSHRSKRSLSCRPPMVKLVCPADNPRAEGLECAKTCQNYDLQCMSTGCVSGCLCPQGMVRHENRCVALERCPCFHQGQEYAPGETVKIDCNTCVCRDRKWNCTDHVCDATCSAIGMAHYLTFDGLKYLFPGECQYVLVQDYCGSNPGTFRILVGNEGCSYPSVKCKKRVTILVEGGEIELFDGEVNVKKPMKDETHFEVVESGQYVILLLGKALSVVWDHRLSISVTLKRTYQEQVCGLCGNFDGIQNNDFTSSSLQIEEDPVDFGNSWKVNPQCADTKKVPLDSSPAVCHNNIMKQTMVDSSCRILTSDIFQDCNRLVDPEPFLDICIYDTCSCESIGDCTCFCDTIAAYAHVCAQHGKVVAWRTATFCPQNCEERNLHENGYECEWRYNSCAPACPITCQHPEPLACPVQCVEGCHAHCPPGKILDELLQTCIDPEDCPVCEVAGRRLAPGKKIILNPSDPEHCQICHCDGVNFTCQACREPGSLVVPPTEGPIGSTTSYVEDTPEPPLHDFHCSRLLDLVFLLDGSSKLSEDEFEVLKVFVVGMMEHLHISQKRIRVAVVEYHDGSHAYIELKDRKRPSELRRITSQVKYAGSEVASTSEVLKYTLFQIFGKIDRPEASRIALLLMASQEPSRLARNLVRYVQGLKKKKVIVIPVGIGPHASLKQIHLIEKQAPENKAFVFSGVDELEQRRDEIINYLCDLAPEAPAPTQHPPMAQVTVGSELLGVSSPGPKRNSMVLDVVFVLEGSDKIGEANFNKSREFMEEVIQRMDVGQDRIHVTVLQYSYMVTVEYTFSEAQSKGEVLQQVRDIRYRGGNRTNTGLALQYLSEHSFSVSQGDREQVPNLVYMVTGNPASDEIKRMPGDIQVVPIGVGPHANVQELEKIGWPNAPILIHDFEMLPREAPDLVLQRCCSGEGLQIPTLSPTPDCSQPLDVVLLLDGSSSIPASYFDEMKSFTKAFISRANIGPRLTQVSVLQYGSITTIDVPWNVAYEKVHLLSLVDLMQQEGGPSQIGDALSFAVRYVTSEVHGARPGASKAVVILVTDVSVDSVDAAAEAARSNRVTVFPIGIGDRYSEAQLSSLAGPKAGSNMVRLQRIEDLPTVATLGNSFFHKLCSGFDRVCVDEDGNEKRPGDVWTLPDQCHTVTCLPDGQTLLKSHRVNCDRGPRPSCPNGQPPLRVEETCGCRWTCPCVCMGSSTRHIVTFDGQNFKLTGSCSYVLFQNKEQDLEVILHNGACSPGAKETCMKSIEVKHDGLSVELHSDMQMTVNGRLVSIPYVGGDMEVNVYGTIMYEVRFNHLGHIFTFTPQNNEFQLQLSPRTFASKTYGLCGICDENGANDFILRDGTVTTDWKALIQEWTVQQLGKTCQPVPEEQCPVSSSSHCQVLLSELFAECHKVLAPATFYAMCQPDSCHPKKVCEAIALYAHLCRTKGVCVDWRRANFCAMSCPPSLVYNHCEHGCPRLCEGNTSSCGDQPSEGCFCPPNQVMLEGSCVPEEACTQCISEDGVRHQFLETWVPAHQPCQICTCLSGRKVNCTLQPCPTARAPTCGPCEVARLRQNAEQCCPEYECVCDLVSCDLPPVPPCEDGLQMTLTNPGECRPNFTCACRKDECRRESPPSCPPHRTLALRKTQCCDEYECACNCVNSTVSCPLGYLASAVTNDCGCTTTTCFPDKVCVHRGTIYPVGQFWEEACDVCTCTDLEDSVMGLRVAQCSQKPCEDNCLSGFTYVLHEGECCGRCLPSACEVVIGSPRGDAQSHWKNVGSHWASPDNPCLINECVRVKEEVFVQQRNVSCPQLNVPTCPTGFQLSCKTSECCPTCHCEPLEACLLNGTIIGPGKSLMIDVCTTCRCTVQVGVISGFKLECRKTTCEACPLGYKEEKNQGECCGRCLPIACTIQLRGGQIMTLKRDETIQDGCDSHFCKVNERGEYIWEKRVTGCPPFDEHKCLAEGGKIMKIPGTCCDTCEEPECKDIIAKLQRVKVGDCKSEEEVDIHYCEGKCASKAVYSIHMEDVQDQCSCCSPTQTEPMQVPLRCTNGSLIYHEILNAMQCRCSPRKCSK

SEQ ID NO: 4 - protein, 2813 aa, Mus musculus
MNPFRYEICLLVLALTWPGTLCTEKPRDRPSTARCSLFGDDFINTFDETMYSFAGGCSYLLAGDCQKRSFSILGNFQDGKRMSLSVYLGEFFDIHLFANGTVMQGDQSISMPYASQGLYLELEAGYYKLSSETFGFAARIDGNGNFQVLMSDRHFNKTCGLCGDFNIFAEDDFRTQEGTLTSDPYDFANSWALSSEEQRCKRASPPSRNCESSSGDMHQAMWEQCQLLKTASVFARCHPLVDPESFVALCEKILCTCATGPECACPVLLEYARTCAQEGMVLYGWTDHSACRPACPAGMEYKECVSPCPRTCQSLSINEVCQQQCVDGCSCPEGELLDEDRCVQSSDCPCVHAGKRYPPGTSLSQDCNTCICRNSLWICSNEECPGECLVTGQSHFKSFDNRYFTFSGICQYLLARDCEDHTFSIVIETMQCADDPDAVCTRSVSVRLSALHNSLVKLKHGGAVGIDGQDVQLPFLQGDLRIQHTVMASVRLSYAEDLQMDWDGRGRLLVKLSPVYSGKTCGLCGNYNGNKGDDFLTPAGLVEPLVVDFGNAWKLQGDCSDLRRQHSDPCSLNPRLTRFAEEACALLTSSKFEACHHAVSPLPYLQNCRYDVCSCSDSRDCLCNAVANYAAECARKGVHIGWREPGFCALGCPQGQVYLQCGNSCNLTCRSLSLPDEECSEVCLEGCYCPPGLYQDERGDCVPKAQCPCYYDGELFQPADIFSDHHTMCYCEDGFMHCTTSGTLGSLLPDTVLSSPLSHRSKRSLSCRPPMVKLVCPADNPRAQGLECAKTCQNYDLERMSLGCVSGCLCPPGMVRHENKCVALERCPCFHQGAEYAPGDTVKIGCNTCVCRERKWNCTNHVCDATRSAIGMAHYLTFDGLKYLFPGECQYVLVYDYCGSNPGTFQILVGNEGCSYPSVKCRKRVTILVDGGELELFDGEVNVKRPLRDESHFEVVESGRYVILLLGQALSVVWDHHLSISVVLKHTYQEQVCGLCGNFDGIQNNDSTTSSLQVEEDPVNFGNSWKVSSQCADTRKLSLDVSPATCHNNIMKQTMVDSACRILTSDVFQGCNRLVDPEPYLDICIYDTCSCESIGDCACFCDTIAAYAHVCAQHGQVVAWRTPTLCPQSCEEKNVRENGYECEWRYNSCAPACPVTCQHPEPLACPVQCVEGCHAHCPPGRILDELLQTCVDPQDCPVCEVAGRRLAPGKKITLSPDDPAHCQNCHCDGVNLTCEACQEPGGLVAPPTDAPVSSTTPYVEDTPEPPLHNFYCSKLLDLVFLLDGSSMLSEAEFEVLKAFVVGMMERLHISQKRIRVAVVEYHDGSRAYLELKARKRPSELRRITSQIKYTGSQVASTSEVLKYTLFQIFGKIDRPEASHITLLLTASQEPPRMARNLVRYVQGLKKKKVIVIPVGIGPHASLKQIRLIEKQAPENKAFLLSGVDELEQRRDEIVSYLCDLAPEAPAPTQPPQVAHVTVSPGIAGISSPGPKRKSMVLDVVFVLEGSDEVGEANFNKSKEFVEEVIQRMDVSPDATRISVLQYSYTVTMEYAFNGAQSKEEVLRHVREIRYQGGNRTNTGQALQYLSEHSFSPSQGDRVEAPNLVYMVTGNPASDEIKRLPGDIQVVPIGVGPHANMQELERISRPIAPIFIRDFETLPREAPDLVLQTCCSKEGLQLPTLPPLPDCSQPLDVVLLLDGSSSLPESSFDKMKSFAKAFISKANIGPHLTQVSVIQYGSINTIDVPWNVVQEKAHLQSLVDLMQQEGGPSQIGDALAFAVRYVTSQIHGARPGASKAVVIIIMDTSLDPVDTAADAARSNRVAVFPVGVGDRYDEAQLRILAGPGASSNVVKLQQVEDLSTMATLGNSFFHKLCSGFSGVCVDEDGNEKRPGDVWTLPDQCHTVTCLANGQTLLQSHRVNCDHGPRPSCANSQSPVRVEETCGCRWTCPCVCTGSSTRHIVTFDGQNFKLTGSCSYVIFQNKEQDLEVLLHNGACSPGAKQACMKSIEIKHAGVSAELHSNMEMAVDGRLVLAPYVGENMEVSIYGAIMYEVRFTHLGHILTYTPQNNEFQLQLSPKTFASKMHGLCGICDENGANDFTLRDGTVTTDWKRLVQEWTVQQPGYTCQAVPEEQCPVSDSSHCQVLLSASFAECHKVIAPATFHTICQQDSCHQERVCEVIASYAHLCRTSGVCVDWRTTDFCAMSCPPSLVYNHCERGCPRHCDGNTSFCGDHPSEGCFCPQHQVFLEGSCVPEEACTQCVGEDGVRHQFLETWVPDHQPCQICMCLSGRKINCTAQPCPTARAPTCGPCEVARLKQSTNLCCPEYECVCDLFNCNLPPVPPCEGGLQPTLTNPGECRPTFTCDCRKEECKRVSPPSCPPHRTPTLRKTQCCDEYECACSCVNSTLSCPLGYLASATTNDCGCTTTTCLPDKVCVHRGTVYPVGQFWEEGCDTCTCTDMEDTVVGLRVVQCSQRPCEDSCQPGFSYVLHEGECCGRCLPSACKVVAGSLRGDSHSSWKSVGSRWAVPENPCLVNECVRVEDAVFVQQRNISCPQLAVPTCPTGFQLNCETSECCPSCHCEPVEACLLNGTIIGPGKSVMVDLCTTCRCIVQTDAISRFKLECRKTTCEACPMGYREEKSQGECCGRCLPTACTIQLRGGRIMTLKQDETFQDGCDSHLCRVNERGEYIWEKRVTGCPPFDEHKCLAEGGKIVKIPGTCCDTCEEPDCKDITAKVQYIKVGDCKSQEEVDIHYCQGKCASKAVYSIDIEDVQEQCSCCLPSRTEPMRVPLHCTNGSVVYHEVINAMQCRCSPRNCSK

SEQ ID NO: 5 - protein, 90 aa, Homo sapiens
CNDITARLQYVKVGSCKSEVEVDIHYCQGKCASKAMYSIDINDVQDQCSCCSPTRTEPMQVALHCTNGSVVYHEVLNAMECKCSPRKCSK

SEQ ID NO: 6 - protein, 90 aa, unidentified
  source 1..90: note = CK domain of a non-human primate (NHP) VWF protein
CSDITARLQYVKVGSCKSEVEVDIHYCQGKCASKAMYSIDINDVQDQCSCCSPTRTEPMQVPLHCTNGSVVYHEVLNAMQCECSPRKCSK

SEQ ID NO: 7 - protein, 150 aa, synthetic construct
MIPARFAGVLLALALILPGTLCAEHHHHHHGSGLNDIFEAQKIEWHEGSLEVLFQGPEPECNDITARLQYVKVGSCKSEVEVDIHYCQGKCASKAMYSIDINDVQDQCSCCSPTRTEPMQVALHCTNGSVVYHEVLNAMECKCSPRKCSK

SEQ ID NO: 8 - protein, 150 aa, synthetic construct
MIPARFAGVLLALALILPGTLCAEHHHHHHGSGLNDIFEAQKIEWHEGSLEVLFQGPEPECSDITARLQYVKVGSCKSEVEVDIHYCQGKCASKAMYSIDINDVQDQCSCCSPTRTEPMQVPLHCTNGSVVYHEVLNAMQCECSPRKCSK

SEQ ID NO: 9 - protein, 150 aa, synthetic construct
MIPARFAGVLLALALILPGTLCAEHHHHHHGSGLNDIFEAQKIEWHEGSLEVLFQGPEPECKDIIAKLQRVKVGDCKSEEEVDIHYCEGKCASKAVYSIHMEDVQDQCSCCSPTQTEPMQVPLRCTNGSLIYHEILNAMQCRCSPRKCSK

SEQ ID NO: 10 - protein, 150 aa, synthetic construct
MIPARFAGVLLALALILPGTLCAEHHHHHHGSGLNDIFEAQKIEWHEGSLEVLFQGPEPDCKDITAKVQYIKVGDCKSQEEVDIHYCQGKCASKAVYSIDIEDVQEQCSCCLPSRTEPMRVPLHCTNGSVVYHEVINAMQCRCSPRNCSK

SEQ ID NO: 11 - protein, 6 aa, synthetic construct
HHHHHH

SEQ ID NO: 12 - protein, 121 aa, synthetic construct
QVQLVQSGAEVKKPGASVKVSCKVSGDTLSSYGIHWVRQAPGKGLEWMGGSIPSFGTAIYAQKFQGRVTMTEDTSTDTAYMELSSLKSEDTAVYYCTSEGPWLPFFSFDYWGQGTLVTVSS

SEQ ID NO: 13 - protein, 8 aa, synthetic construct
GDTLSSYG

SEQ ID NO: 14 - protein, 8 aa, synthetic construct
SIPSFGTA

SEQ ID NO: 15 - protein, 14 aa, synthetic construct
TSEGPWLPFFSFDY

SEQ ID NO: 16 - protein, 107 aa, synthetic construct
DIQMTQSPSSVSASVGDRVTITCRASQGISRNLAWYQQKPGKAPKLLIYKASSLASGVPSRFSGSGSGTDFTLTISSLQPEDFANYYCQQSFSTPVTFGGGTKVEIK

SEQ ID NO: 17 - protein, 6 aa, synthetic construct
QGISRN

SEQ ID NO: 18 - protein, 9 aa, synthetic construct
QQSFSTPVT

SEQ ID NO: 19 - protein, 118 aa, synthetic construct
QVQLVQSGAEVKKPGASVKVSCKVSGYTSRQYIHWVRQAPGKGLEWMGWVTPIFGTTNYAQKFQGRVTMTEDTSTDTAYMELSSLKSEDTAVYYCAREWTGTTAFDIWGQGTLVTVSS

SEQ ID NO: 20 - protein, 7 aa, synthetic construct
GYTSRQY

SEQ ID NO: 21 - protein, 8 aa, synthetic construct
VTPIFGTT

SEQ ID NO: 22 - protein, 12 aa, synthetic construct
AREWTGTTAFDI

SEQ ID NO: 23 - protein, 107 aa, synthetic construct
DIQMTQSPSSVSASVGDRVTITCRASQDIGHYLAWYQQKPGKAPKLLIYAASNRASGVPSRFSGSGSGTDFTLTISSLQPEDFANYYCQQGYSAPYGFGGGTKVEIK

SEQ ID NO: 24 - protein, 6 aa, synthetic construct
QDIGHY

SEQ ID NO: 25 - protein, 9 aa, synthetic construct
QQGYSAPYG

SEQ ID NO: 26 - protein, 120 aa, synthetic construct
QVQLVQSGAEVKKPGASVKVSCKVSGVTLTSSAISWVRQAPGKGLEWMGGITPIFGTPIYAQKFQGRVTMTEDTSTDTAYMELSSLKSEDTAVYYCATDYGYPFWAFDIWGQGTMVTVSS

SEQ ID NO: 27 - protein, 8 aa, synthetic construct
GVTLTSSA

SEQ ID NO: 28 - protein, 8 aa, synthetic construct
ITPIFGTP

SEQ ID NO: 29 - protein, 13 aa, synthetic construct
ATDYGYPFWAFDI

SEQ ID NO: 30 - protein, 107 aa, synthetic construct
DIQMTQSPSSVSASVGDRVTITCRASQGIYSALAWYQQKPGKAPKLLIYESSKRPSGVPSRFSGSGSGTDFTLTISSLQPEDFANYYCEQYASSPPTFGGGTKVEIK

SEQ ID NO: 31 - protein, 6 aa, synthetic construct
QGIYSA

SEQ ID NO: 32 - protein, 9 aa, synthetic construct
EQYASSPPT

SEQ ID NO: 33 - protein, 121 aa, synthetic construct
EVQLVESGGGLVQPGRSLRLSCAASGFNFHNYDMHWVRQAPGKGLEWVSAISGSGYTADYADSVEGRFTISRDNAKNSLYLQMNSLRAEDTAVYYCAKSHSSGWSSAFDIWGQGTMVTVSS

SEQ ID NO: 34 - protein, 8 aa, synthetic construct
GFNFHNYD

SEQ ID NO: 35 - protein, 8 aa, synthetic construct
ISGSGYTA

SEQ ID NO: 36 - protein, 14 aa, synthetic construct
AKSHSSGWSSAFDI

SEQ ID NO: 37 - protein, 108 aa, synthetic construct
DIQMTQSPSSLSASVGDRVTITCRASQTIGYWLAWYQQKPGKAPKLLIYDATNLATGVPSRFSGSGSGTDFTLTISSLQPEDVATYYCQHYNPYSQVTFGGGTKVEIK

SEQ ID NO: 38 - protein, 6 aa, synthetic construct
QTIGYW

SEQ ID NO: 39 - protein, 10 aa, synthetic construct
QHYNPYSQVT

SEQ ID NO: 40 - protein, 126 aa, synthetic construct
QVQLVQSGAEVKKPGASVKVSCKVSGSTFSNYAIHWVRQAPGKGLEWMGGIVPLSGTAIYAQKFQGRVTMTEDTSTDTAYMELSSLKSEDTAVYYCARDQGESSGWYAIYYGMDVWGQGTTVTVSS

SEQ ID NO: 41 - protein, 8 aa, synthetic construct
GSTFSNYA

SEQ ID NO: 42 - protein, 8 aa, synthetic construct
IVPLSGTA

SEQ ID NO: 43 - protein, 19 aa, synthetic construct
ARDQGESSGWYAIYYGMDV

SEQ ID NO: 44 - protein, 106 aa, synthetic construct
DIQMTQSPSSVSASVGDRVTITCRASQGIGTWLAWYQQKPGKAPKLLIYGSSSRASGVPSRFSGSGSGTDFTLTISSLQPEDFANYYCQQSYGKPTFGGGTKVEIK

SEQ ID NO: 45 - protein, 6 aa, synthetic construct
QGIGTW

SEQ ID NO: 46 - protein, 8 aa, synthetic construct
QQSYGKPT

SEQ ID NO: 47 - protein, 125 aa, synthetic construct
QVQLQESGPGLVKPSQTLSLTCTVSGYSISSGDYHWSWIRQPPGKGLEWIGYINPSGSADYNPSLKSRVTMSVDTSKNQFSLKVNSVTAADTAVYYCARDGVAATYYYYYGMDVWGQGTTVTVSS

SEQ ID NO: 48 - protein, 10 aa, synthetic construct
GYSISSGDYH

SEQ ID NO: 49 - protein, 7 aa, synthetic construct
INPSGSA

SEQ ID NO: 50 - protein, 17 aa, synthetic construct
ARDGVAATYYYYYGMDV

SEQ ID NO: 51 - protein, 107 aa, synthetic construct
EIVMTQSPATLSLSPGERATLSCRASHSIGSNLAWYQQKPGQAPRLLIYDATTRATGIPARFSGSGSGTDFTLTISSLEPEDFAVYYCQQAYSFPLTFGGGTKVEIK

SEQ ID NO: 52 - protein, 6 aa, synthetic construct
HSIGSN

SEQ ID NO: 53 - protein, 9 aa, synthetic construct
QQAYSFPLT

SEQ ID NO: 54 - protein, 125 aa, synthetic construct
EVQLVESGGGLVQPGRSLRLSCAASGVDFSTYAMHWVRQAPGKGLEWVSAISSSGAYTDYADSVEGRFTISRDNAKNSLYLQMNSLRAEDTAVYYCATDRGPFSGYGTQGWFDPWGQGTLVTVSS

SEQ ID NO: 55 - protein, 8 aa, synthetic construct
GVDFSTYA

SEQ ID NO: 56 - protein, 8 aa, synthetic construct
ISSSGAYT

SEQ ID NO: 57 - protein, 18 aa, synthetic construct
ATDRGPFSGYGTQGWFDP

SEQ ID NO: 58 - protein, 107 aa, synthetic construct
DIQMTQSPSSLSASVGDRVTITCRASQTIRSYLAWYQQKPGKAPKLLIYGASDLQSGVPSRFSGSGSGTDFTLTISSLQPEDVATYYCQQYANFPITFGGGTKVEIK

SEQ ID NO: 59 - protein, 6 aa, synthetic construct
QTIRSY

SEQ ID NO: 60 - protein, 9 aa, synthetic construct
QQYANFPIT

SEQ ID NO: 61 - protein, 121 aa, synthetic construct; identical to SEQ ID NO: 33

SEQ ID NO: 62 - protein, 8 aa, synthetic construct
GFNFHNYD

SEQ ID NO: 63 - protein, 8 aa, synthetic construct
ISGSGYTA

SEQ ID NO: 64 - protein, 14 aa, synthetic construct
AKSHSSGWSSAFDI

SEQ ID NO: 65 - protein, 107 aa, synthetic construct
DIQMTQSPSSLSASVGDRVTITCRASQTIGYWLAWYQQKPGKAPKLLIYDASVPESGVPSRFSGSGSGTDFTLTISSLQPEDVATYYCQQTLSSPWTFGGGTKVEIK

SEQ ID NO: 66 - protein, 6 aa, synthetic construct
QTIGYW

SEQ ID NO: 67 - protein, 9 aa, synthetic construct
QQTLSSPWT

SEQ ID NO: 68 - protein, 124 aa, synthetic construct
EVQLVESGGGLVQPGRSLRLSCAASGFSFSMYAMHWVRQAPGKGLEWVSAISPSGDATDYADSVEGRFTISRDNAKNSLYLQMNSLRAEDTAVYYCARGMQDYGHYYYYYMDVWGKGTTVTVSS

SEQ ID NO: 69 - protein, 8 aa, synthetic construct
GFSFSMYA

SEQ ID NO: 70 - protein, 8 aa, synthetic construct
ISPSGDAT

SEQ ID NO: 71 - protein, 17 aa, synthetic construct
ARGMQDYGHYYYYYMDV

SEQ ID NO: 72 - protein, 107 aa, synthetic construct
DIQMTQSPSSLSASVGDRVTITCRASQDISTSLAWYQQKPGKAPKLLIYGASSLETGVPSRFSGSGSGTDFTLTISSLQPEDVATYYCQQGYIAPRTFGGGTKVEIK

SEQ ID NO: 73 - protein, 6 aa, synthetic construct
QDISTS

SEQ ID NO: 74 - protein, 9 aa, synthetic construct
QQGYIAPRT

SEQ ID NO: 75 - protein, 125 aa, synthetic construct; identical to SEQ ID NO: 54

SEQ ID NO: 76 - protein, 8 aa, synthetic construct
GVDFSTYA

SEQ ID NO: 77 - protein, 8 aa, synthetic construct
ISSSGAYT

SEQ ID NO: 78 - protein, 18 aa, synthetic construct
ATDRGPFSGYGTQGWFDP

SEQ ID NO: 79 - protein, 107 aa, synthetic construct
DIQMTQSPSSLSASVGDRVTITCRASRDITSYLAWYQQKPGKAPKLLIYHASTLEAGVPSRFSGSGSGTDFTLTISSLQPEDVATYYCQQYGDFPPTFGGGTKVEIK

SEQ ID NO: 80 - protein, 6 aa, synthetic construct
RDITSY

SEQ ID NO: 81 - protein, 9 aa, synthetic construct
QQYGDFPPT

SEQ ID NO: 82 - protein, 126 aa, synthetic construct
QVQLQESGPGLVKPSQTLSLTCTVSGSTFGDYAWSWIRQPPGKGLEWIGYINHIGNADYNPSLKSRVTMSVDTSKNQFSLKVNSVTAADTAVYYCARDGVESPEVPNYYYYGMDVWGQGTTVTVSS

SEQ ID NO: 83 - protein, 8 aa, synthetic construct
GSTFGDYA

SEQ ID NO: 84 - protein, 7 aa, synthetic construct
INHIGNA

SEQ ID NO: 85 - protein, 20 aa, synthetic construct
ARDGVESPEVPNYYYYGMDV

SEQ ID NO: 86 - protein, 106 aa, synthetic construct
EIVMTQSPATLSLSPGERATLSCRASHSIGSNLAWYQQKPGQAPRLLIYDATNRAPGIPARFSGSGSGTDFTLTISSLEPEDFAVYYCQQSYTNPTFGGGTKVEIK

SEQ ID NO: 87 - protein, 6 aa, synthetic construct
HSIGSN

SEQ ID NO: 88 - protein, 8 aa, synthetic construct
QQSYTNPT

SEQ ID NO: 89 - protein, 119 aa, synthetic construct
EVQLVESGGGLVQPGRSLRLSCAASGFNFHNYDMHWVRQAPGKGLEWVSAISGGAVSTDYADSVEGRFTISRDNAKNSLYLQMNSLRAEDTAVYYCARSSWYGGQIDFWGQGTLVTVSS

SEQ ID NO: 90 - protein, 8 aa, synthetic construct
GFNFHNYD

SEQ ID NO: 91 - protein, 8 aa, synthetic construct
ISGGAVST

SEQ ID NO: 92 - protein, 12 aa, synthetic construct
ARSSWYGGQIDF

SEQ ID NO: 93 - protein, 107 aa, synthetic construct
DIQMTQSPSSLSASVGDRVTITCRASESVSSWLAWYQQKPGKAPKLLIYGASTLEGGVPSRFSGSGSGTDFTLTISSLQPEDVATYYCQQAYRTPVTFGGGTKVEIK

SEQ ID NO: 94 - protein, 6 aa, synthetic construct
ESVSSW

SEQ ID NO: 95 - protein, 9 aa, synthetic construct
QQAYRTPVT

SEQ ID NO: 96 - protein, 124 aa, synthetic construct
QVQLQESGPGLVKPSQTLSLTCTVSGSSISSSSDYWSWIRQPPGKGLEWIGYIDISGDADYNPSLKSRVTMSVDTSKNQFSLKVNSVTAADTAVYYCARHLSSGWYGTYYFDYWGQGTLVTVSS

SEQ ID NO: 97 - protein, 10 aa, synthetic construct
GSSISSSSDY

SEQ ID NO: 98 - protein, 7 aa, synthetic construct
IDISGDA

SEQ ID NO: 99 - protein, 16 aa, synthetic construct
ARHLSSGWYGTYYFDY

SEQ ID NO: 100 - protein, 107 aa, synthetic construct
EIVMTQSPATLSLSPGERATLSCRASQNIHSNLAWYQQKPGQAPRLLIYDASSRATGIPARFSGSGSGTDFTLTISSLEPEDFAVYYCQQGYTKPLTFGGGTKVEIK

SEQ ID NO: 101 - protein, 6 aa, synthetic construct
QNIHSN

SEQ ID NO: 102 - protein, 9 aa, synthetic construct
QQGYTKPLT

SEQ ID NO: 103 - protein, 120 aa, synthetic construct
QVQLVQSGAEVKKPGASVKVSCKVSGDTFTDRYIHWVRQAPGKGLEWMGGINTIFGTAIYAQKFQGRVTMTEDTSTDTAYMELSSLKSEDTAVYYCAREGATVSTNFDYWGQGTLVTVSS

SEQ ID NO: 104 - protein, 8 aa, synthetic construct
GDTFTDRY

SEQ ID NO: 105 - protein, 8 aa, synthetic construct
INTIFGTA

SEQ ID NO: 106 - protein, 13 aa, synthetic construct
AREGATVSTNFDY

SEQ ID NO: 107 - protein, 107 aa, synthetic construct
DIQMTQSPSSVSASVGDRVTITCRASQDIRKSLAWYQQKPGKAPKLLIYDISNRPSGVPSRFSGSGSGTDFTLTISSLQPEDFANYYCQQGYSAPYGFGGGTKVEIK

SEQ ID NO: 108 - protein, 6 aa, synthetic construct
QDIRKS

SEQ ID NO: 109 - protein, 9 aa, synthetic construct
QQGYSAPYG

SEQ ID NO: 110 - protein, 123 aa, synthetic construct
QVQLVQSGAEVKKPGASVKVSCKVSGYTFASHYIHWVRQAPGKGLEWMGGMDPATGQTIYAQKFQGRVTMTEDTSTDTAYMELSSLKSEDTAVYYCAKGDRSSWYEVLSFQHWGQGTLVTVSS

SEQ ID NO: 111 - protein, 8 aa, synthetic construct
GYTFASHY

SEQ ID NO: 112 - protein, 8 aa, synthetic construct
MDPATGQT

SEQ ID NO: 113 - protein, 16 aa, synthetic construct
AKGDRSSWYEVLSFQH

SEQ ID NO: 114 - protein, 107 aa, synthetic construct
DIQMTQSPSSVSASVGDRVTITCRASQGISTDLAWYQQKPGKAPKLLIYDASILQRGVPSRFSGSGSGTDFTLTISSLQPEDFANYYCQQVRTYPFTFGGGTKVEIK

SEQ ID NO: 115 - protein, 6 aa, synthetic construct
QGISTD

SEQ ID NO: 116 - protein, 9 aa, synthetic construct
QQVRTYPFT

SEQ ID NO: 117 - protein, 124 aa, synthetic construct
QVQLVQSGAEVKKPGASVKVSCKVSGYPFTSSGIHWVRQAPGKGLEWMGGVIPLFGTANYAQKFQGRVTMTEDTSTDTAYMELSSLKSEDTAVYYCARIRVSSGWYRDYGMDVWGQGTTVTVSS

SEQ ID NO: 118 - protein, 8 aa, synthetic construct
GYPFTSSG

SEQ ID NO: 119 - protein, 8 aa, synthetic construct
VIPLFGTA

SEQ ID NO: 120 - protein, 17 aa, synthetic construct
ARIRVSSGWYRDYGMDV

SEQ ID NO: 121 - protein, 107 aa, synthetic construct
DIQMTQSPSSVSASVGDRVTITCRASQDITNDLAWYQQKPGKAPKLLIYDASRLQSGVPSRFSGSGSGTDFTLTISSLQPEDFANYYCQQTFTTPLTFGGGTKVEIK

SEQ ID NO: 122 - protein, 6 aa, synthetic construct
QDITND

SEQ ID NO: 123 - protein, 9 aa, synthetic construct
QQTFTTPLT

SEQ ID NO: 124 - protein, 126 aa, synthetic construct
QVQLVQSGAEVKKPGASVKVSCKVSGYTFTSHIIHWVRQAPGKGLEWMGGMNPYSNKAIYAQKFQGRVTMTEDTSTDTAYMELSSLKSEDTAVYYCARDASDYGDYGYYYYYMDVWGKGTTVTVSS

SEQ ID NO: 125 - protein, 8 aa, synthetic construct
GYTFTSHI

SEQ ID NO: 126 - protein, 8 aa, synthetic construct
MNPYSNKA

SEQ ID NO: 127 - protein, 19 aa, synthetic construct
ARDASDYGDYGYYYYYMDV

SEQ ID NO: 128 - protein, 107 aa, synthetic construct
DIQMTQSPSSVSASVGDRVTITCRASQYIGNDLAWYQQKPGKAPKLLIYEASQRPSGVPSRFSGSGSGTDFTLTISSLQPEDFANYYCQQQYNYPLTFGGGTKVEIK

SEQ ID NO: 129 - protein, 6 aa, synthetic construct
QYIGND

SEQ ID NO: 130 - protein, 9 aa, synthetic construct
QQQYNYPLT

SEQ ID NO: 131 - protein, 126 aa, synthetic construct
QVQLVQSGAEVKKPGASVKVSCKVSGDTLSNYAIHWVRQAPGKGLEWMGGINPNAGTTIYAQKFQGRVTMTEDTSTDTAYMELSSLKSEDTAVYYCARDSSSWCPVGYYYYYMDVWGKGTTVTVSS

SEQ ID NO: 132 - protein, 8 aa, synthetic construct
GDTLSNYA

SEQ ID NO: 133 - protein, 8 aa, synthetic construct
INPNAGTT

SEQ ID NO: 134 - protein, 19 aa, synthetic construct
ARDSSSWCPVGYYYYYMDV

SEQ ID NO: 135 - protein, 107 aa, synthetic construct
DIQMTQSPSSVSASVGDRVTITCRASQSIGDFLAWYQQKPGKAPKLLIYDASHLQTGVPSRFSGSGSGTDFTLTISSLQPEDFANYYCQQTSTIPPTFGGGTKVEIK

SEQ ID NO: 136 - protein, 6 aa, synthetic construct
QSIGDF

SEQ ID NO: 137 - protein, 9 aa, synthetic construct
QQTSTIPPT

SEQ ID NO: 138 - protein, 124 aa, synthetic construct
QVQLQESGPGLVKPSQTLSLTCTVSGESFSGHFWSWIRQPPGKGLEWIGYISQSGRTDYNPSLKSRVTMSVDTSKNQFSLKVNSVTAADTAVYYCARTLPRIQLWSPRGAFDIWGQGTMVTVSS

SEQ ID NO: 139 - protein, 8 aa, synthetic construct
GESFSGHF

SEQ ID NO: 140 - protein, 7 aa, synthetic construct
ISQSGRT

SEQ ID NO: 141 - protein, 18 aa, synthetic construct
ARTLPRIQLWSPRGAFDI

SEQ ID NO: 142 - protein, 107 aa, synthetic construct
EIVMTQSPATLSLSPGERATLSCRASQSLGRSLAWYQQKPGQAPRLLIYDASHRATGIPARFSGSGSGTDFTLTISSLEPEDFAVYYCQQYVSYPLTFGGGTKVEIK

SEQ ID NO: 143 - protein, 6 aa, synthetic construct
QSLGRS

SEQ ID NO: 144 - protein, 9 aa, synthetic construct
QQYVSYPLT

SEQ ID NO: 145 - protein, 122 aa, synthetic construct
QVQLQESGPGLVKPSQTLSLTCTVSGASLGDQYWSWIRQPPGKGLEWIGYIHPSGDFDYNPSLKSRVTMSVDTSKNQFSLKVNSVTAADTAVYYCARHYSSSWYGTWYFDLWGRGTLVTVSS

SEQ ID NO: 146 - protein, 8 aa, synthetic construct
GASLGDQY

SEQ ID NO: 147 - protein, 7 aa, synthetic construct
IHPSGDF

SEQ ID NO: 148 - protein, 16 aa, synthetic construct
ARHYSSSWYGTWYFDL

SEQ ID NO: 149 - protein, 107 aa, synthetic construct
EIVMTQSPATLSLSPGERATLSCRASQNIGTNLAWYQQKPGQAPRLLIYDASVRAAGIPARFSGSGSGTDFTLTISSLEPEDFAVYYCQQSLTTPLTFGGGTKVEIK

SEQ ID NO: 150 - protein, 6 aa, synthetic construct
QNIGTN

SEQ ID NO: 151 - protein, 9 aa, synthetic construct
QQSLTTPLT

SEQ ID NO: 152 - protein, 126 aa, synthetic construct; identical to SEQ ID NO: 131

SEQ ID NO: 153 - protein, 8 aa, synthetic construct
GDTLSNYA

SEQ ID NO: 154 - protein, 8 aa, synthetic construct
INPNAGTT

SEQ ID NO: 155 - protein, 19 aa, synthetic construct
ARDSSSWCPVGYYYYYMDV

SEQ ID NO: 156 - protein, 107 aa, synthetic construct; identical to SEQ ID NO: 135

SEQ ID NO: 157 - protein, 6 aa, synthetic construct
QSIGDF

SEQ ID NO: 158 - protein, 9 aa, synthetic construct
QQTSTIPPT

SEQ ID NO: 159 - protein, 125 aa, synthetic construct
QVQLQESGPGLVKPSQTLSLTCTVSGASISTHSWSWIRQPPGKGLEWIGYINPSGDIDYNPSLKSRVTMSVDTSKNQFSLKVNSVTAADTAVYYCARGRTGFDSSGFVVGAFDIWGQGTMVTVSS

SEQ ID NO: 160 - protein, 8 aa, synthetic construct
GASISTHS

SEQ ID NO: 161 - protein, 7 aa, synthetic construct
INPSGDI

SEQ ID NO: 162 - protein, 19 aa, synthetic construct
ARGRTGFDSSGFVVGAFDI

SEQ ID NO: 163 - protein, 107 aa, synthetic construct
EIVMTQSPATLSLSPGERATLSCRASQNVNDNLAWYQQKPGQAPRLLIYDASTRPTGIPARFSGSGSGTDFTLTISSLEPEDFAVYYCQQYLTIPRTFGGGTKVEIK

SEQ ID NO: 164 - protein, 6 aa, synthetic construct
QNVNDN

SEQ ID NO: 165 - protein, 9 aa, synthetic construct
QQYLTIPRT

SEQ ID NO: 166 - protein, 119 aa, synthetic construct
EVQLVESGGGLVQPGRSLRLSCAASEFTFGDYDMHWVRQAPGKGLEWVSAISSSGSNRDYADSVEGRFTISRDNAKNSLYLQMNSLRAEDTAVYYCARDAWFGDAFDIWGQGTMVTVSS

SEQ ID NO: 167 - protein, 8 aa, synthetic construct
EFTFGDYD

SEQ ID NO: 168 - protein, 8 aa, synthetic construct
ISSSGSNR

SEQ ID NO: 169 - protein, 12 aa, synthetic construct
ARDAWFGDAFDI

SEQ ID NO: 170 - protein, 106 aa, synthetic construct
DIQMTQSPSSLSASVGDRVTITCRASESVSSWLAWYQQKPGKAPKLLIYGASTLEGGVPSRFSGSGSGTDFTLTISSLQPEDVATYYCQQSYSFPDFGGGTKVEIK

SEQ ID NO: 171 - protein, 6 aa, synthetic construct
ESVSSW

SEQ ID NO: 172 - protein, 8 aa, synthetic construct
QQSYSFPD

SEQ ID NO: 173 - protein, 125 aa, synthetic construct
QVQLVQSGAEVKKPGASVKVSCKVSGYTLTDLPIHWVRQAPGKGLEWMGGINPFLGTTIYAQKFQGRVTMTEDTSTDTAYMELSSLKSEDTAVYYCARGVDMLATARDYYYMAIWGKGTTVTVSS

SEQ ID NO: 174 - protein, 8 aa, synthetic construct
GYTLTDLP

SEQ ID NO: 175 - protein, 8 aa, synthetic construct
INPFLGTT

SEQ ID NO: 176 - protein, 18 aa, synthetic construct
ARGVDMLATARDYYYMAI

SEQ ID NO: 177 - protein, 107 aa, synthetic construct
DIQMTQSPSSVSASVGDRVTITCRASQSIGNDLAWYQQKPGKAPKLLIYGASMRATGVPSRFSGSGSGTDFTLTISSLQPEDFANYYCQQYASDPLTFGGGTKVEIK

SEQ ID NO: 178 - protein, 6 aa, synthetic construct
QSIGND

SEQ ID NO: 179 - protein, 9 aa, synthetic construct
QQYASDPLT

SEQ ID NO: 180 - protein, 118 aa, synthetic construct
QVQLQESGPGLVKPSQTLSLTCTVSGASFGDNYWSWIRQPPGKGLEWIGYIDTSGSSDYNPSLKSRVTMSVDTSKNQFSLKVNSVTAADTAVYYCARVSWGYYAMDVWGQGTTVTVSS

SEQ ID NO: 181 - protein, 8 aa, synthetic construct
GASFGDNY

SEQ ID NO: 182 - protein, 7 aa, synthetic construct
IDTSGSS

SEQ ID NO: 183 - protein, 12 aa, synthetic construct
ARVSWGYYAMDV

SEQ ID NO: 184 - protein, 108 aa, synthetic construct
EIVMTQSPATLSLSPGERATLSCRASQGISGRLAWYQQKPGQAPRLLIYDVSTRATGIPARFSGSGSGTDFTLTISSLEPEDFAVYYCQQYDTSAPLTFGGGTKVEIK

SEQ ID NO: 185 - protein, 6 aa, synthetic construct
QGISGR

SEQ ID NO: 186 - protein, 10 aa, synthetic construct
QQYDTSAPLT

SEQ ID NO: 187 - protein, 125 aa, synthetic construct
QVQLQESGPGLVKPSQTLSLTCTVSGVTFSSYGWSWIRQPPGKGLEWIGYVDTSGSTDYNPSLKSRVTMSVDTSKNQFSLKVNSVTAADTAVYYCARTTYYYDSSGFARGWFDPWGQGTLVTVSS

SEQ ID NO: 188 - protein, 8 aa, synthetic construct
GVTFSSYG

SEQ ID NO: 189 - protein, 7 aa, synthetic construct
VDTSGST

SEQ ID NO: 190 - protein, 19 aa, synthetic construct
ARTTYYYDSSGFARGWFDP

SEQ ID NO: 191 - protein, 109 aa, synthetic construct
EIVMTQSPATLSLSPGERATLSCRASQSVPSSHLAWYQQKPGQAPRLLIYDATNRAAGIPARFSGSGSGTDFTLTISSLEPEDFAVYYCQQYDNWPSPSFGGGTKVEIK

SEQ ID NO: 192 - protein, 7 aa, synthetic construct
QSVPSSH

SEQ ID NO: 193 - protein, 10 aa, synthetic construct
QQYDNWPSPS

SEQ ID NO: 194 - protein, 126 aa, synthetic construct
QVQLVQSGAEVKKPGASVKVSCKVSGDTLSNYAIHWVRQAPGKGLEWMGGINPNAGTTIYAQKFQGRVTMTEDTSTDTAYMELSSLKSEDTAVYYCARDSSSWSQFGYYYYYMDVWGKGTTVTVSS

SEQ ID NO: 195 - protein, 8 aa, synthetic construct
GDTLSNYA

SEQ ID NO: 196 - protein, 8 aa, synthetic construct
INPNAGTT

SEQ ID NO: 197 - protein, 19 aa, synthetic construct
ARDSSSWSQFGYYYYYMDV

SEQ ID NO: 198 - protein, 107 aa, synthetic construct; identical to SEQ ID NO: 135

SEQ ID NO: 199 - protein, 6 aa, synthetic construct
QSIGDF

SEQ ID NO: 200 - protein, 9 aa, synthetic construct
QQTSTIPPT

SEQ ID NO: 201 - protein, 118 aa, synthetic construct
EVQLVQSGAEVKKPGASVKVSCKASGDTFSSHVISWVRQAPGQGLEWMGGINPRTGTTIYAQKLQGRGTMTTDPSTSTAYMELRSLRSDDTAVYYCARTRPWAGLDVWGQGTTVTVSS

SEQ ID NO: 202 - protein, 8 aa, synthetic construct
GDTFSSHV

SEQ ID NO: 203 - protein, 8 aa, synthetic construct
INPRTGTT

SEQ ID NO: 204 - protein, 11 aa, synthetic construct
ARTRPWAGLDV

SEQ ID NO: 205 - protein, 106 aa, synthetic construct
ESALTQPASVSGSPGQSITISCTGTNIEYKGVSWYQQHPGKAPKLMIYGVDKRPSGVSNRFSGSKSGNTASLTISGLQAEDEADYYCQAWGTSTAIFGGGTKLTVL

SEQ ID NO: 206 - protein, 6 aa, synthetic construct
NIEYKG

SEQ ID NO: 207 - protein, 9 aa, synthetic construct
QAWGTSTAI

SEQ ID NO: 208 - protein, 126 aa, synthetic construct; identical to SEQ ID NO: 131

SEQ ID NO: 209 - protein, 8 aa, synthetic construct
GDTLSNYA

SEQ ID NO: 210 - protein, 8 aa, synthetic construct
INPNAGTT

SEQ ID NO: 211 - protein, 19 aa, synthetic construct
ARDSSSWCPVGYYYYYMDV

SEQ ID NO: 212 - protein, 107 aa, synthetic construct; identical to SEQ ID NO: 135

SEQ ID NO: 213 - protein, 6 aa, synthetic construct
QSIGDF

SEQ ID NO: 214 - protein, 9 aa, synthetic construct
QQTSTIPPT

SEQ ID NO: 215 - protein, 124 aa, synthetic construct
QVQLQESGPGLVKPSQTLSLTCTVSGVSLSTSDYSWSWIRQPPGKGLEWIGYISPPGRSDYNPSLKSRVTMSVDTSKNQFSLKVNSVTAADTAVYYCARHSSSGWYSTYDFDSWGQGTMVTVSS

SEQ ID NO: 216 - protein, 10 aa, synthetic construct
GVSLSTSDYS

SEQ ID NO: 217 - protein, 7 aa, synthetic construct
ISPPGRS

SEQ ID NO: 218 - protein, 16 aa, synthetic construct
ARHSSSGWYSTYDFDS

SEQ ID NO: 219 - protein, 107 aa, synthetic construct
EIVMTQSPATLSLSPGERATLSCRASQNIHSNLAWYQQKPGQAPRLLIYGATTRATGIPARFSGSGSGTDFTLTISSLEPEDFAVYYCQQSYTTPLTFGGGTKVEIK

SEQ ID NO: 220 - protein, 6 aa, synthetic construct
QNIHSN

SEQ ID NO: 221 - protein, 9 aa, synthetic construct
QQSYTTPLT

SEQ ID NO: 222 - protein, 126 aa, synthetic construct; identical to SEQ ID NO: 124

SEQ ID NO: 223 - protein, 8 aa, synthetic construct
GYTFTSHI

SEQ ID NO: 224 - protein, 8 aa, synthetic construct
MNPYSNKA

SEQ ID NO: 225 - protein, 19 aa, synthetic construct
ARDASDYGDYGYYYYYMDV

SEQ ID NO: 226 - protein, 107 aa, synthetic construct; identical to SEQ ID NO: 135

SEQ ID NO: 227 - protein, 6 aa, synthetic construct
QSIGDF

SEQ ID NO: 228 - protein, 9 aa, synthetic construct
QQTSTIPPT

SEQ ID NO: 229 - protein, 126 aa, synthetic construct; identical to SEQ ID NO: 131

SEQ ID NO: 230 - protein, 8 aa, synthetic construct
GDTLSNYA

SEQ ID NO: 231 - protein, 8 aa, synthetic construct
INPNAGTT

SEQ ID NO: 232 - protein, 19 aa, synthetic construct
ARDSSSWCPVGYYYYYMDV

SEQ ID NO: 233 - protein, 107 aa, synthetic construct; identical to SEQ ID NO: 135

SEQ ID NO: 234 - protein, 6 aa, synthetic construct
QSIGDF

SEQ ID NO: 235 - protein, 9 aa, synthetic construct
QQTSTIPPT

SEQ ID NO: 236 - protein, 119 aa, synthetic construct
EVQLVESGGGLVQPGRSLRLSCAASGFTVSSNAMHWVRQAPGKGLEWVSAIGTFADTDYADSVEGRFTISRDNAKNSLYLQMNSLRAEDTAVYYCAREGAAAGTNFDYWGQGTLVTVSS

SEQ ID NO: 237 - protein, 8 aa, synthetic construct
GFTVSSNA

SEQ ID NO: 238 - protein, 7 aa, synthetic construct
IGTFADT

SEQ ID NO: 239 - protein, 13 aa, synthetic construct
AREGAAAGTNFDY

SEQ ID NO: 240 - protein, 107 aa, synthetic construct
DIQMTQSPSSLSASVGDRVTITCRASQGIRSYLAWYQQKPGKAPKLLIYGASTLEGGVPSRFSGSGSGTDFTLTISSLQPEDVATYYCQQSYSTPPKFGGGTKVEIK

SEQ ID NO: 241 - protein, 6 aa, synthetic construct
QGIRSY

SEQ ID NO: 242 - protein, 9 aa, synthetic construct
QQSYSTPPK

SEQ ID NO: 243 - protein, 127 aa, synthetic construct
QVQLVQSGAEVKKPGASVKVSCKVSGYAFPSYGIHWVRQAPGKGLEWMGGISAFTGDTIYAQKFQGRVTMTEDTSTDTAYMELSSLKSEDTAVYYCASGPTNVLTGLMESAEYFQHWGQGTLVTVSS

SEQ ID NO: 244 - protein, 8 aa, synthetic construct
GYAFPSYG

SEQ ID NO: 245 - protein, 8 aa, synthetic construct
ISAFTGDT

SEQ ID NO: 246 - protein, 20 aa, synthetic construct
ASGPTNVLTGLMESAEYFQH

SEQ ID NO: 247 - protein, 107 aa, synthetic construct
DIQMTQSPSSVSASVGDRVTITCRASQGISRDLAWYQQKPGKAPKLLIYGASGRATGVPSRFSGSGSGTDFTLTISSLQPEDFANYYCQQSYITPTTFGGGTKVEIK

SEQ ID NO: 248 - protein, 6 aa, synthetic construct
QGISRD

SEQ ID NO: 249 - protein, 9 aa, synthetic construct
QQSYITPTT

SEQ ID NO: 250 - protein, 119 aa, synthetic construct
QVQLQESGPGLVKPSQTLSLTCTVSGESFSTSYWSWIRQPPGKGLEWIGYITHSGYTDYNPSLKSRVTMSVDTSKNQFSLKVNSVTAADTAVYYCARGGRFPFWYFDLWGRGSLVTVSS

SEQ ID NO: 251 - protein, 8 aa, synthetic construct
GESFSTSY

SEQ ID NO: 252 - protein, 7 aa, synthetic construct
ITHSGYT

SEQ ID NO: 253 - protein, 13 aa, synthetic construct
ARGGRFPFWYFDL

SEQ ID NO: 254 - protein, 106 aa, synthetic construct
EIVMTQSPATLSLSPGERATLSCRASRSIGSNLAWYQQKPGQAPRLLIYGATTRATGIPARFSGSGSGTDFTLTISSLEPEDFAVYYCQQYKNDATFGGGTKVEIK

SEQ ID NO: 255 - protein, 6 aa, synthetic construct
RSIGSN

SEQ ID NO: 256 - protein, 8 aa, synthetic construct
QQYKNDAT

SEQ ID NO: 257 - protein, 125 aa, synthetic construct
QVQLVQSGAEVKKPGASVKVSCKVSEGTLSRHYINWVRQAPGKGLEWMGGINAKSGATIYAQKFQGRVTMTEDTSTDTAYMELSSLKSEDTAVYYCAKDTGDDILTDYSDAFDIWGQGTMVTVSS

SEQ ID NO: 258 - protein, 8 aa, synthetic construct
EGTLSRHY

SEQ ID NO: 259 - protein, 8 aa, synthetic construct
INAKSGAT

SEQ ID NO: 260 - protein, 18 aa, synthetic construct
AKDTGDDILTDYSDAFDI

SEQ ID NO: 261 - protein, 109 aa, synthetic construct
DIQMTQSPSSVSASVGDRVTITCRASQNIGTSLAWYQQKPGKAPKLLIYGASNLESGVPSRFSGSGSGTDFTLTISSLQPEDFANYYCLQYNDWPPLYSFGGGTKVEIK

SEQ ID NO: 262 - protein, 6 aa, synthetic construct
QNIGTS

SEQ ID NO: 263 - protein, 11 aa, synthetic construct
LQYNDWPPLYS

SEQ ID NO: 264 - protein, 129 aa, synthetic construct
QVQLQESGPGLVKPSQTLSLTCTVSGASISTSDHSWSWIRQPPGKGLEWIGYIRDTGSTDYNPSLKSRVTMSVDTSKNQFSLKVNSVTAADTAVYYCAISSTERWKQSFGVDTEYFQHWGQGTLVTVSS

SEQ ID NO: 265 - protein, 10 aa, synthetic construct
GASISTSDHS

SEQ ID NO: 266 - protein, 7 aa, synthetic construct
IRDTGST

SEQ ID NO: 267 - protein, 21 aa, synthetic construct
AISSTERWKQSFGVDTEYFQH

SEQ ID NO: 268 - protein, 107 aa, synthetic construct
EIVMTQSPATLSLSPGERATLSCRASRYVSTNLAWYQQKPGQAPRLLIYDAVTRAPGIPARFSGSGSGTDFTLTISSLEPEDFAVYYCQQYHSLPLIFGGGTKVEIK

SEQ ID NO: 269 - protein, 6 aa, synthetic construct
RYVSTN

SEQ ID NO: 270 - protein, 9 aa, synthetic construct
QQYHSLPLI

SEQ ID NO: 271 - protein, 126 aa, synthetic construct; identical to SEQ ID NO: 131

SEQ ID NO: 272 - protein, 8 aa, synthetic construct
GDTLSNYA

SEQ ID NO: 273 - protein, 8 aa, synthetic construct
INPNAGTT

SEQ ID NO: 274 - protein, 19 aa, synthetic construct
ARDSSSWCPVGYYYYYMDV

SEQ ID NO: 275 - protein, 107 aa, synthetic construct; identical to SEQ ID NO: 135

SEQ ID NO: 276 - protein, 6 aa, synthetic construct
QSIGDF

SEQ ID NO: 277 - protein, 9 aa, synthetic construct
QQTSTIPPT

SEQ ID NO: 278 - protein, 126 aa, synthetic construct; identical to SEQ ID NO: 131

SEQ ID NO: 279 - protein, 8 aa, synthetic construct
GDTLSNYA

SEQ ID NO: 280 - protein, 8 aa, synthetic construct
INPNAGTT

SEQ ID NO: 281 - protein, 19 aa, synthetic construct
ARDSSSWCPVGYYYYYMDV

SEQ ID NO: 282 - protein, 107 aa, synthetic construct; identical to SEQ ID NO: 135

SEQ ID NO: 283 - protein, 6 aa, synthetic construct
QSIGDF

SEQ ID NO: 284 - protein, 9 aa, synthetic construct
QQTSTIPPT

SEQ ID NO: 285 - protein, 119 aa, synthetic construct
EVQLVESGGGLVQPGRSLRLSCAASGFRFDEYDMHWVRQAPGKGLEWVSAISSESASIDYADSVEGRFTISRDNAKNSLYLQMNSLRAEDTAVYYCARDGAYGDYFDYWGQGTMVTVSS

SEQ ID NO: 286 - protein, 8 aa, synthetic construct
GFRFDEYD

SEQ ID NO: 287 - protein, 8 aa, synthetic construct
ISSESASI

SEQ ID NO: 288 - protein, 12 aa, synthetic construct
ARDGAYGDYFDY

SEQ ID NO: 289 - protein, 107 aa, synthetic construct
DIQMTQSPSSLSASVGDRVTITCRASQDIKSWLAWYQQKPGKAPKLLIYGASTLQSGVPSRFSGSGSGTDFTLTISSLQPEDVATYYCQQSYSAPYTFGGGTKVEIK

SEQ ID NO: 290 - protein, 6 aa, synthetic construct
QDIKSW

SEQ ID NO: 291 - protein, 9 aa, synthetic construct
QQSYSAPYT

SEQ ID NO: 292 - protein, 119 aa, synthetic construct
QVQLVQSGAEVKKPGASVKVSCKVSGAPSAAIIHWVRQAPGKGLEWMGGFTQIFGTAIYAQKFQGRVTMTEDTSTDTAYMELSSLKSEDTAVYYCAREGIGGHDAFDIWGQGTMVTVSS

SEQ ID NO: 293 - protein, 7 aa, synthetic construct
GAPSAAI

SEQ ID NO: 294 - protein, 8 aa, synthetic construct
FTQIFGTA

SEQ ID NO: 295 - protein, 13 aa, synthetic construct
AREGIGGHDAFDI

SEQ ID NO: 296 - protein, 107 aa, synthetic construct
DIQMTQSPSSVSASVGDRVTITCRASQDISKNLAWYQQKPGKAPKLLIYKASKLESGVPSRFSGSGSGTDFTLTISSLQPEDFANYYCLHDYTYPPTFGGGTKVEIK

SEQ ID NO: 297 - protein, 6 aa, synthetic construct
QDISKN

SEQ ID NO: 298 - protein, 9 aa, synthetic construct
LHDYTYPPT

SEQ ID NO: 299 - protein, 124 aa, synthetic construct
QVQLVQSGAEVKKPGASVKVSCKVSGYTLTSNYIHWVRQAPGKGLEWMGGFNPSSGSTIYAQKFQGRVTMTEDTSTDTAYMELSSLKSEDTAVYYCARTLYDFWSGYLDAFDIWGQGTMVTVSS

SEQ ID NO: 300 - protein, 8 aa, synthetic construct
GYTLTSNY

SEQ ID NO: 301 - protein, 8 aa, synthetic construct
FNPSSGST

SEQ ID NO: 302 - protein, 17 aa, synthetic construct
ARTLYDFWSGYLDAFDI

SEQ ID NO: 303 - protein, 107 aa, synthetic construct
DIQMTQSPSSVSASVGDRVTITCRASQNIRSSLAWYQQKPGKAPKLLIYAASNLEPGVPSRFSGSGSGTDFTLTISSLQPEDFANYYCQQYEDTPLTFGGGTKVEIK

SEQ ID NO: 304 - protein, 6 aa, synthetic construct
QNIRSS

SEQ ID NO: 305 - protein, 9 aa, synthetic construct
QQYEDTPLT

SEQ ID NO: 306 - protein, 123 aa, synthetic construct
QVQLVQSGAEVKKPGASVKVSCKVSGITFTSDYIHWVRQAPGKGLEWMGGMNPYSNKAIYAQKFQGRVTMTEDTSTDTAYMELSSLKSEDTAVYYCATGGLRIAARTSYFDYWGQGTLVTVSS

SEQ ID NO: 307 - protein, 8 aa, synthetic construct
GITFTSDY

SEQ ID NO: 308 - protein, 8 aa, synthetic construct
MNPYSNKA

SEQ ID NO: 309 - protein, 16 aa, synthetic construct
ATGGLRIAARTSYFDY

SEQ ID NO: 310 - protein, 108 aa, synthetic construct
DIQMTQSPSSVSASVGDRVTITCRASMDIAGSLAWYQQKPGKAPKLLIYDTSFRATGVPSRFSGSGSGTDFTLTISSLQPEDFANYYCQQYDNPPRLTFGGGTKVEIK

SEQ ID NO: 311 - protein, 6 aa, synthetic construct
MDIAGS

SEQ ID NO: 312 - protein, 10 aa, synthetic construct
QQYDNPPRLT

SEQ ID NO: 313 - protein, 118 aa, synthetic construct
QVQLQESGPGLVKPSQTLSLTCTVSGASLGHQYWSWIRQPPGKGLEWIGYIDTSGSTDYNPSLKSRVTMSVDTSKNQFSLKVNSVTAADTAVYYCARVANYYYYMDVWGKGTTVTVSS

SEQ ID NO: 314 - protein, 8 aa, synthetic construct
GASLGHQY

SEQ ID NO: 315 - protein, 7 aa, synthetic construct
IDTSGST

SEQ ID NO: 316 - protein, 12 aa, synthetic construct
ARVANYYYYMDV

SEQ ID NO: 317 - protein, 107 aa, synthetic construct
EIVMTQSPATLSLSPGERATLSCRASQSVSNRLAWYQQKPGQAPRLLIYDASNRATGIPARFSGSGSGTDFTLTISSLEPEDFAVYYCQQYFITPSTFGGGTKVEIK

SEQ ID NO: 318 - protein, 6 aa, synthetic construct
QSVSNR

SEQ ID NO: 319 - protein, 9 aa, synthetic construct
QQYFITPST

SEQ ID NO: 320 - protein, 119 aa, synthetic construct
EVQLVQSGAEVKKPGASVKVSCKASGYRFTNYDISWVRQAPGQGLEWMGGINPSSGITIYAQKLQGRGTMTTDPSTSTAYMELRSLRSDDTAVYYCARGAAYGDYLDYWGQGTLVTVSS

SEQ ID NO: 321 - protein, 8 aa, synthetic construct
GYRFTNYD

SEQ ID NO: 322 - protein, 8 aa, synthetic construct
INPSSGIT

SEQ ID NO: 323 - protein, 12 aa, synthetic construct
ARGAAYGDYLDY

SEQ ID NO: 324 - protein, 109 aa, synthetic construct
ESALTQPASVSGSPGQSITISCTGTNLGEKYVSWYQQHPGKAPKLMIYSTSQRPSGVSNRFSGSKSGNTASLTITGLQAEDEADYYCSSYTASSTPGLVFGGGTKLTVL

SEQ ID NO: 325 - protein, 6 aa, synthetic construct
NLGEKY

SEQ ID NO: 326 - protein, 12 aa, synthetic construct
SSYTASSTPGLV

SEQ ID NO: 327 - protein, 118 aa, synthetic construct
QVQLQESGPGLVKPSQTLSLTCTVSGASFGDNYWSWIRQPPGKGLEWIGYIDTSGSSDYNPSLKSRVTMSVDTSKNQFSLKVNSVTAADTAVYYCARDALGYYYMDVWGKGTTVTVSS

SEQ ID NO: 328 - protein, 8 aa, synthetic construct
GASFGDNY

SEQ ID NO: 329 - protein, 7 aa, synthetic construct
IDTSGSS

SEQ ID NO: 330 - protein, 12 aa, synthetic construct
ARDALGYYYMDV

SEQ ID NO: 331 - protein, 107 aa, synthetic construct
EIVMTQSPATLSLSPGERATLSCRASQGISGRLAWYQQKPGQAPRLLIYDASARASGIPARFSGSGSGTDFTLTISSLEPEDFAVYYCQQYGTSPYTFGGGTKVEIK

SEQ ID NO: 332 - protein, 6 aa, synthetic construct
QGISGR

SEQ ID NO: 333 - protein, 9 aa, synthetic construct
QQYGTSPYT

SEQ ID NO: 334 - protein, 118 aa, synthetic construct
QVQLQESGPGLVKPSQTLSLTCTVSGASFGDHYWSWIRQPPGKGLEWIGYIDATGSIDYNPSLKSRVTMSVDTSKNQFSLKVNSVTAADTAVYYCARVASYYYYMDVWGKGTTVTVSS

SEQ ID NO: 335 - protein, 8 aa, synthetic construct
GASFGDHY

SEQ ID NO: 336 - protein, 7 aa, synthetic construct
IDATGSI

SEQ ID NO: 337 - protein, 12 aa, synthetic construct
ARVASYYYYMDV

SEQ ID NO: 338 - protein, 108 aa, synthetic construct
EIVMTQSPATLSLSPGERATLSCRASQSITGSYLAWYQQKPGQAPRLLIYGASDRAPGIPARFSGSGSGTDFTLTISSLEPEDFAVYYCQQYGSFPYTFGGGTKVEIK

SEQ ID NO: 339 - protein, 7 aa, synthetic construct
QSITGSY

SEQ ID NO: 340 - protein, 9 aa, synthetic construct
QQYGSFPYT

SEQ ID NO: 341 - protein, 123 aa, synthetic construct
EVQLVESGGGLVQPGRSLRLSCAASGFTFADYDMHWVRQAPGKGLEWVSATSSHGVTTDYADSVEGRFTISRDNAKNSLYLQMNSLRAEDTAVYYCARGAWSGIAARQYIDYWGQGTLVTVSS

SEQ ID NO: 342 - protein, 8 aa, synthetic construct
GFTFADYD

SEQ ID NO: 343 - protein, 8 aa, synthetic construct
TSSHGVTT

SEQ ID NO: 344 - protein, 16 aa, synthetic construct
ARGAWSGIAARQYIDY

SEQ ID NO: 345 - protein, 107 aa, synthetic construct
DIQMTQSPSSLSASVGDRVTITCRASQTIGYWLAWYQQKPGKAPKLLIYDASVPESGVPSRFSGSGSGTDFTLTISSLQPEDVATYYCQQSYSYPLTFGGGTKVEIK

SEQ ID NO: 346 - protein, 6 aa, synthetic construct
QTIGYW

SEQ ID NO: 347 - protein, 9 aa, synthetic construct
QQSYSYPLT

SEQ ID NO: 348 - protein, 125 aa, synthetic construct; identical to SEQ ID NO: 187

SEQ ID NO: 349 - protein, 8 aa, synthetic construct
GVTFSSYG

SEQ ID NO: 350 - protein, 7 aa, synthetic construct
VDTSGST

SEQ ID NO: 351 - protein, 19 aa, synthetic construct
ARTTYYYDSSGFARGWFDP

SEQ ID NO: 352 - protein, 107 aa, synthetic construct
EIVMTQSPATLSLSPGERATLSCRASRSISTNLAWYQRKPGQAPRLLIYDTSNRAPGIPARFSGSGSGTDFTLTISSLEPEDFAVYYCLQYDVSPYTFGGGTKVEIK

SEQ ID NO: 353 - protein, 6 aa, synthetic construct
RSISTN

SEQ ID NO: 354 - protein, 9 aa, synthetic construct
LQYDVSPYT

SEQ ID NO: 355 - protein, 119 aa, synthetic construct
EVQLVESGGGLVQPGRSLRLSCAASGFNFHNYDMHWVRQAPGKGLEWVSAISGSSDTMDYADSVEGRFTISRDNAKNSLYLQMNSLRAEDTAVYYCVRASWYGTFFDYWGQGTLVTVSS

SEQ ID NO: 356 - protein, 8 aa, synthetic construct
GFNFHNYD

SEQ ID NO: 357 - protein, 8 aa, synthetic construct
ISGSSDTM

SEQ ID NO: 358 - protein, 12 aa, synthetic construct
VRASWYGTFFDY

SEQ ID NO: 359 - protein, 107 aa, synthetic construct
DIQMTQSPSSLSASVGDRVTITCRASQDISGWLAWYQQKPGKAPKLLIYAASSLQDGVPSRFSGSGSGTDFTLTISSLQPEDVATYYCQQSYRIPSTFGGGTKVEIK

SEQ ID NO: 360 - protein, 6 aa, synthetic construct
QDISGW

SEQ ID NO: 361 - protein, 9 aa, synthetic construct
QQSYRIPST

SEQ ID NO: 362 - protein, 119 aa, synthetic construct; identical to SEQ ID NO: 89

SEQ ID NO: 363 - protein, 8 aa, synthetic construct
GFNFHNYD

SEQ ID NO: 364 - protein, 8 aa, synthetic construct
ISGGAVST

SEQ ID NO: 365 - protein, 12 aa, synthetic construct
ARSSWYGGQIDF

SEQ ID NO: 366 - protein, 107 aa, synthetic construct; identical to SEQ ID NO: 359

SEQ ID NO: 367 - protein, 6 aa, synthetic construct
QDISGW

SEQ ID NO: 368 - protein, 9 aa, synthetic construct
QQSYRIPST

SEQ ID NO: 369 - protein, 119 aa, synthetic construct
EVQLVESGGGLVQPGRSLRLSCAASAFTFSNYDMHWVRQAPGKGLEWVSAISGSGDTSDYADSVEGRFTISRDNAKNSLYLQMNSLRAEDTAVYYCVRASWYGTFFDYWGQGTLVTVSS

SEQ ID NO: 370 - protein, 8 aa, synthetic construct
AFTFSNYD

SEQ ID NO: 371 - protein, 8 aa, synthetic construct
ISGSGDTS

SEQ ID NO: 372 - protein, 12 aa, synthetic construct
VRASWYGTFFDY

SEQ ID NO: 373 - protein, 107 aa, synthetic construct
DIQMTQSPSSLSASVGDRVTITCRASQSVSGWLAWYQQKPGKAPKLLIYGTSTLQSGVPSRFSGSGSGTDFTLTISSLQPEDVATYYCQQSYSAPYTFGGGTKVEIK

SEQ ID NO: 374 - protein, 6 aa, synthetic construct
QSVSGW

SEQ ID NO: 375 - protein, 9 aa, synthetic construct
QQSYSAPYT

SEQ ID NO: 376 - protein, 118 aa, synthetic construct
QVQLQESGPGLVKPSQTLSLTCTVSGVSISNHYWSWIRQPPGKGLEWIGYIDESGSTDYNPSLKSRVTMSVDTSKNQFSLKVNSVTAADTAVYYCARVSWGYYAMDVWGQGTTVTVSS

SEQ ID NO: 377 - protein, 8 aa, synthetic construct
GVSISNHY

SEQ ID NO: 378 - protein, 7 aa, synthetic construct
IDESGST

SEQ ID NO: 379 - protein, 12 aa, synthetic construct
ARVSWGYYAMDV

SEQ ID NO: 380 - protein, 107 aa, synthetic construct
EIVMTQSPATLSLSPGERATLSCRASQGVSGHLAWYQQKPGQAPRLLIYETSKRATGIPARFSGSGSGTDFTLTISSLEPEDFAVYYCQQYNDYPLGFGGGTKVEIK

SEQ ID NO: 381 - protein, 6 aa, synthetic construct
QGVSGH

SEQ ID NO: 382 - protein, 9 aa, synthetic construct
QQYNDYPLG

SEQ ID NO: 383 - protein, 120 aa, synthetic construct
QVQLQESGPGLVKPSQTLSLTCTVSGFSLTSHPEGVNWIRQPPGKGLEWIGYIAHSGTTDYNPSLKSRVTMSVDTSKNQFSLKVNSVTAADTAVYYCARAYGDSDAFDIWGQGTMVTVSS

SEQ ID NO: 384 - protein, 10 aa, synthetic construct
GFSLTSHPEG

SEQ ID NO: 385 - protein, 7 aa, synthetic construct
IAHSGTT

SEQ ID NO: 386 - protein, 12 aa, synthetic construct
ARAYGDSDAFDI

SEQ ID NO: 387 - protein, 106 aa, synthetic construct
EIVMTQSPATLSLSPGERATLSCRASQSIGRNLAWYQQKPGQAPRLLIYGASKRVTGIPARFSGSGSGTDFTLTISSLEPEDFAVYYCQQYSTWPTFGGGTKVEIK

SEQ ID NO: 388 - protein, 6 aa, synthetic construct
QSIGRN

SEQ ID NO: 389 - protein, 8 aa, synthetic construct
QQYSTWPT

SEQ ID NO: 390 - protein, 124 aa, synthetic construct; identical to SEQ ID NO: 117

SEQ ID NO: 391 - protein, 8 aa, synthetic construct
GYPFTSSG

SEQ ID NO: 392 - protein, 8 aa, synthetic construct
VIPLFGTA

SEQ ID NO: 393 - protein, 17 aa, synthetic construct
ARIRVSSGWYRDYGMDV

SEQ ID NO: 394 - protein, 107 aa, synthetic construct; identical to SEQ ID NO: 135

SEQ ID NO: 395 - protein, 6 aa, synthetic construct
QSIGDF

SEQ ID NO: 396 - protein, 9 aa, synthetic construct
QQTSTIPPT

SEQ ID NO: 397 - protein, 119 aa, synthetic construct
EVQLVESGGGLVQPGRSLRLSCAASGFNFHNYDMHWVRQAPGKGLEWVSAIDISGQSTDYADSVEGRFTISRDNAKNSLYLQMNSLRAEDTAVYYCAREGWYGGVFDYWGQGTLVTVSS

SEQ ID NO: 398 - protein, 8 aa, synthetic construct
GFNFHNYD

SEQ ID NO: 399 - protein, 8 aa, synthetic construct
IDISGQST

SEQ ID NO: 400 - protein, 12 aa, synthetic construct
AREGWYGGVFDY

SEQ ID NO: 401 - protein, 106 aa, synthetic construct
DIQMTQSPSSLSASVGDRVTITCRASQDIANWLAWYQQKPGKAPKLLIYGASESGVGVPSRFSGSGSGTDFTLTISSLQPEDVATYYCQQSLTMKTFGGGTKVEIK

SEQ ID NO: 402 - protein, 6 aa, synthetic construct
QDIANW

SEQ ID NO: 403 - protein, 8 aa, synthetic construct
QQSLTMKT

SEQ ID NO: 404 - protein, 126 aa, synthetic construct; identical to SEQ ID NO: 131

SEQ ID NO: 405 - protein, 8 aa, synthetic construct
GDTLSNYA

SEQ ID NO: 406 - protein, 8 aa, synthetic construct
INPNAGTT

SEQ ID NO: 407 - protein, 19 aa, synthetic construct
ARDSSSWCPVGYYYYYMDV

SEQ ID NO: 408 - protein, 107 aa, synthetic construct; identical to SEQ ID NO: 135

SEQ ID NO: 409 - protein, 6 aa, synthetic construct
QSIGDF

SEQ ID NO: 410 - protein, 9 aa, synthetic construct
QQTSTIPPT

SEQ ID NO: 411 - protein, 120 aa, synthetic construct
QVQLVQSGAEVKKPGASVKVSCKVSGYTFSSSGIHWVRQAPGKGLEWMGGIFPNFGTAIYAQKFQGRVTMTEDTSTDTAYMELSSLKSEDTAVYYCARESDFWSGSFTNWGQGTLVTVSS

SEQ ID NO: 412 - protein, 8 aa, synthetic construct
GYTFSSSG

SEQ ID NO: 413 - protein, 8 aa, synthetic construct
IFPNFGTA

SEQ ID NO: 414 - protein, 13 aa, synthetic construct
ARESDFWSGSFTN

SEQ ID NO: 415 - protein, 109 aa, synthetic construct
DIQMTQSPSSVSASVGDRVTITCRASRSIGNWLAWYQQKPGKAPKLLIYDAFNRASGVPSRFSGSGSGTDFTLTISSLQPEDFANYYCQQSYSTLPRYSFGGGTKVEIK

SEQ ID NO: 416 - protein, 6 aa, synthetic construct
RSIGNW

SEQ ID NO: 417 - protein, 11 aa, synthetic construct
QQSYSTLPRYS

SEQ ID NO: 418 - protein, 126 aa, synthetic construct; identical to SEQ ID NO: 131

SEQ ID NO: 419 - protein, 8 aa, synthetic construct
GDTLSNYA

SEQ ID NO: 420 - protein, 8 aa, synthetic construct
INPNAGTT

SEQ ID NO: 421 - protein, 19 aa, synthetic construct
ARDSSSWCPVGYYYYYMDV

SEQ ID NO: 422 - protein, 107 aa, synthetic construct; identical to SEQ ID NO: 135

SEQ ID NO: 423 - protein, 6 aa, synthetic construct
QSIGDF

SEQ ID NO: 424 - protein, 9 aa, synthetic construct
QQTSTIPPT

SEQ ID NO: 425 - protein, 126 aa, synthetic construct; identical to SEQ ID NO: 131

SEQ ID NO: 426 - protein, 8 aa, synthetic construct
GDTLSNYA

SEQ ID NO: 427 - protein, 8 aa, synthetic construct
INPNAGTT

SEQ ID NO: 428 - protein, 19 aa, synthetic construct
ARDSSSWCPVGYYYYYMDV

SEQ ID NO: 429 - protein, 107 aa, synthetic construct; identical to SEQ ID NO: 135

SEQ ID NO: 430 - protein, 6 aa, synthetic construct
QSIGDF

SEQ ID NO: 431 - protein, 9 aa, synthetic construct
QQTSTIPPT

SEQ ID NO: 432 - protein, 119 aa, synthetic construct
EVQLVESGGGLVQPGRSLRLSCAASGFSLSTYDMHWVRQAPGKGLEWVSAISSSGDTRDYADSVEGRFTISRDNAKNSLYLQMNSLRAEDTAVYYCARAFWGGDAFDIWGQGTMVTVSS

SEQ ID NO: 433 - protein, 8 aa, synthetic construct
GFSLSTYD

SEQ ID NO: 434 - protein, 8 aa, synthetic construct
ISSSGDTR

SEQ ID NO: 435 - protein, 12 aa, synthetic construct
ARAFWGGDAFDI

SEQ ID NO: 436 - protein, 107 aa, synthetic construct
DIQMTQSPSSLSASVGDRVTITCRASQTIGYWLAWYQQKPGKAPKLLIYGASTLEGGVPSRFSGSGSGTDFTLTISSLQPEDVATYYCQHSYSAPPTFGGGTKVEIK

SEQ ID NO: 437 - protein, 6 aa, synthetic construct
QTIGYW

SEQ ID NO: 438 - protein, 9 aa, synthetic construct
QHSYSAPPT

SEQ ID NO: 439 - protein, 119 aa, synthetic construct
EVQLVESGGGLVQPGRSLRLSCAASGFTFSSDTMHWVRQAPGKGLEWVSAISSSGDHTDYADSVEGRFTISRDNAKNSLYLQMNSLRAEDTAVYYCAREGLRGYYFDYWGQGTLVTVSS

SEQ ID NO: 440 - protein, 8 aa, synthetic construct
GFTFSSDT

SEQ ID NO: 441 - protein, 8 aa, synthetic construct
ISSSGDHT

SEQ ID NO: 442 - protein, 12 aa, synthetic construct
AREGLRGYYFDY

SEQ ID NO: 443 - protein, 107 aa, synthetic construct
DIQMTQSPSSLSASVGDRVTITCRASQGISGHLAWYQQKPGKAPKLLIYAASALQSGVPSRFSGSGSGTDFTLTISSLQPEDVATYYCQQSHSLPWTFGGGTKVEIK

SEQ ID NO: 444 - protein, 6 aa, synthetic construct
QGISGH

SEQ ID NO: 445 - protein, 9 aa, synthetic construct
QQSHSLPWT

SEQ ID NO: 446 - protein, 124 aa, synthetic construct
EVQLVQSGAEVKKPGASVKVSCKASGYTFTDLPISWVRQAPGQGLEWMGWINPYTGDTNYAQKLQGRGTMTTDPSTSTAYMELRSLRSDDTAVYYCARDGVILGYYYYYGMDVWGQGTTVTVSS

SEQ ID NO: 447 - protein, 8 aa, synthetic construct
GYTFTDLP

SEQ ID NO: 448 - protein, 8 aa, synthetic construct
INPYTGDT

SEQ ID NO: 449 - protein, 17 aa, synthetic construct
ARDGVILGYYYYYGMDV

SEQ ID NO: 450 - protein, 107 aa, synthetic construct
ESALTQPASVSGSPGQSITISCTGTDLGSKSVSWYQQHPGKAPKLMIYDNFNRPSGVSNRFSGSKSGNTASLTISGLQAEDEADYYCSSRTVSGTYVFGGGTKLTVL

SEQ ID NO: 451 - protein, 6 aa, synthetic construct
DLGSKS

SEQ ID NO: 452 - protein, 10 aa, synthetic construct
SSRTVSGTYV

SEQ ID NO: 453 - protein, 447 aa, synthetic construct
QVQLVQSGAEVKKPGASVKVSCKVSGDTLSSYGIHWVRQAPGKGLEWMGGSIPSFGTAIYAQKFQGRVTMTEDTSTDTAYMELSSLKSEDTAVYYCTSEGPWLPFFSFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

SEQ ID NO: 454 - protein, 444 aa, synthetic construct
QVQLVQSGAEVKKPGASVKVSCKVSGYTSRQYIHWVRQAPGKGLEWMGWVTPIFGTTNYAQKFQGRVTMTEDTSTDTAYMELSSLKSEDTAVYYCAREWTGTTAFDIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

SEQ ID NO: 455 - protein, 447 aa, synthetic construct
EVQLVESGGGLVQPGRSLRLSCAASGFNFHNYDMHWVRQAPGKGLEWVSAISGSGYTADYADSVEGRFTISRDNAKNSLYLQMNSLRAEDTAVYYCAKSHSSGWSSAFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

SEQ ID NO: 456 - protein, 452 aa, synthetic construct
QVQLVQSGAEVKKPGASVKVSCKVSGSTFSNYAIHWVRQAPGKGLEWMGGIVPLSGTAIYAQKFQGRVTMTEDTSTDTAYMELSSLKSEDTAVYYCARDQGESSGWYAIYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

SEQ ID NO: 457 - protein, 451 aa, synthetic construct
EVQLVESGGGLVQPGRSLRLSCAASGVDFSTYAMHWVRQAPGKGLEWVSAISSSGAYTDYADSVEGRFTISRDNAKNSLYLQMNSLRAEDTAVYYCATDRGPFSGYGTQGWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

SEQ ID NO: 458 - protein, 445 aa, synthetic construct
EVQLVESGGGLVQPGRSLRLSCAASGFNFHNYDMHWVRQAPGKGLEWVSAISGGAVSTDYADSVEGRFTISRDNAKNSLYLQMNSLRAEDTAVYYCARSSWYGGQIDFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

SEQ ID NO: 459 - protein, 449 aa, synthetic construct
QVQLVQSGAEVKKPGASVKVSCKVSGYTFASHYIHWVRQAPGKGLEWMGGMDPATGQTIYAQKFQGRVTMTEDTSTDTAYMELSSLKSEDTAVYYCAKGDRSSWYEVLSFQHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

SEQ ID NO: 460 - protein, 453 aa, synthetic construct
QVQLVQSGAEVKKPGASVKVSCKVSGYAFPSYGIHWVRQAPGKGLEWMGGISAFTGDTIYAQKFQGRVTMTEDTSTDTAYMELSSLKSEDTAVYYCASGPTNVLTGLMESAEYFQHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

SEQ ID NO: 461 - protein, 449 aa, synthetic construct
QVQLVQSGAEVKKPGASVKVSCKVSGITFTSDYIHWVRQAPGKGLEWMGGMNPYSNKAIYAQKFQGRVTMTEDTSTDTAYMELSSLKSEDTAVYYCATGGLRIAARTSYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

SEQ ID NO: 462 - protein, 450 aa, synthetic construct
QVQLVQSGAEVKKPGASVKVSCKVSGYPFTSSGIHWVRQAPGKGLEWMGGVIPLFGTANYAQKFQGRVTMTEDTSTDTAYMELSSLKSEDTAVYYCARIRVSSGWYRDYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

SEQ ID NO: 463 - protein, 214 aa, synthetic construct
DIQMTQSPSSVSASVGDRVTITCRASQGISRNLAWYQQKPGKAPKLLIYKASSLASGVPSRFSGSGSGTDFTLTISSLQPEDFANYYCQQSFSTPVTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 464 - protein, 214 aa, synthetic construct
DIQMTQSPSSVSASVGDRVTITCRASQDIGHYLAWYQQKPGKAPKLLIYAASNRASGVPSRFSGSGSGTDFTLTISSLQPEDFANYYCQQGYSAPYGFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 465 - protein, 215 aa, synthetic construct
DIQMTQSPSSLSASVGDRVTITCRASQTIGYWLAWYQQKPGKAPKLLIYDATNLATGVPSRFSGSGSGTDFTLTISSLQPEDVATYYCQHYNPYSQVTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 466 - protein, 213 aa, synthetic construct
DIQMTQSPSSVSASVGDRVTITCRASQGIGTWLAWYQQKPGKAPKLLIYGSSSRASGVPSRFSGSGSGTDFTLTISSLQPEDFANYYCQQSYGKPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 467 - protein, 214 aa, synthetic construct
DIQMTQSPSSLSASVGDRVTITCRASQTIRSYLAWYQQKPGKAPKLLIYGASDLQSGVPSRFSGSGSGTDFTLTISSLQPEDVATYYCQQYANFPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 468 - protein, 214 aa, synthetic construct
DIQMTQSPSSLSASVGDRVTITCRASESVSSWLAWYQQKPGKAPKLLIYGASTLEGGVPSRFSGSGSGTDFTLTISSLQPEDVATYYCQQAYRTPVTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 469 - protein, 214 aa, synthetic construct
DIQMTQSPSSVSASVGDRVTITCRASQGISTDLAWYQQKPGKAPKLLIYDASILQRGVPSRFSGSGSGTDFTLTISSLQPEDFANYYCQQVRTYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 470 - protein, 214 aa, synthetic construct
DIQMTQSPSSVSASVGDRVTITCRASQGISRDLAWYQQKPGKAPKLLIYGASGRATGVPSRFSGSGSGTDFTLTISSLQPEDFANYYCQQSYITPTTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 471 - protein, 215 aa, synthetic construct
DIQMTQSPSSVSASVGDRVTITCRASMDIAGSLAWYQQKPGKAPKLLIYDTSFRATGVPSRFSGSGSGTDFTLTISSLQPEDFANYYCQQYDNPPRLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 472 - protein, 214 aa, synthetic construct
DIQMTQSPSSVSASVGDRVTITCRASQSIGDFLAWYQQKPGKAPKLLIYDASHLQTGVPSRFSGSGSGTDFTLTISSLQPEDFANYYCQQTSTIPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 473 - protein, 228 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 474 - protein, 228 aa, synthetic construct; identical to SEQ ID NO: 473

SEQ ID NO: 475 - protein, 228 aa, synthetic construct; identical to SEQ ID NO: 473

SEQ ID NO: 476 - protein, 228 aa, synthetic construct; identical to SEQ ID NO: 473

SEQ ID NO: 477 - protein, 228 aa, synthetic construct; identical to SEQ ID NO: 473

SEQ ID NO: 478 - protein, 228 aa, synthetic construct; identical to SEQ ID NO: 473

SEQ ID NO: 479 - protein, 228 aa, synthetic construct; identical to SEQ ID NO: 473

SEQ ID NO: 480 - protein, 228 aa, synthetic construct; identical to SEQ ID NO: 473

SEQ ID NO: 481 - protein, 228 aa, synthetic construct; identical to SEQ ID NO: 473

SEQ ID NO: 482 - protein, 228 aa, synthetic construct; identical to SEQ ID NO: 473

SEQ ID NO: 483 - protein, 21 aa, synthetic construct
SGGSTITSYNVYYTKLSSSGT

SEQ ID NO: 484 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

SEQ ID NO: 485 - protein, 107 aa, Homo sapiens [sequence omitted]

SEQ ID NO: 486 - protein, 159 aa, synthetic construct
MIPARFAGVLLALALILPGTLCAEHHHHHHGSSAWSHPQFEKGSLEVLFQGPEPECNDITARLQYVKVGSCKSEVEVDIHYCQGKCASKAMYSIDINDVQDQCSCCSPTRTEPMQVALHCTNGSVVYHEVLNAMECKCSPRKCSKGGGGSYPYDVPDYA

SEQ ID NO: 487 - protein, 159 aa, synthetic construct
MIPARFAGVLLALALILPGTLCAEHHHHHHGSSAWSHPQFEKGSLEVLFQGPEPECSDITARLQYVKVGSCKSEVEVDIHYCQGKCASKAMYSIDINDVQDQCSCCSPTRTEPMQVPLHCTNGSVVYHEVLNAMQCECSPRKCSKGGGGSYPYDVPDYA